(19) United States
(12) Patent Application Publication
(10) Pub. No.: US 2024/0216513 A1
(43) Pub. Date: Jul. 4, 2024

(54) FATTY ACID-BIFUNCTIONAL DEGRADER CONJUGATES AND THEIR METHODS OF USE

(71) Applicant: Novartis AG, Basel (CH)

(72) Inventors: Claire Adcock, Swaffham (GB); Valerie Broennimann, Basel (CH); Jiashun Cheng, Changshu (CN); Rohit Kumar Duvadie, Arlington, MA (US); Tanzina Fazal, Burlington, MA (US); Jinhai Gao, Belmont, MA (US); Fengfeng Guo, Nantong (CN); Robert Martin Grotzfeld, Ettingen (CH); Christina Hebach, Muenchenstein (CH); Gregory John Hollingworth, Sevenoaks (GB); Darryl Brynley Jones, Basel (CH); Alexei Karpov, Basel (CH); Jialiang Li, Suzhou (CN); Julien Lorber, Colmar (FR); Chester A. Metcalf, III, Needham, MA (US); Walter Michael, Watertown, MA (US); Mark Gabriel Palermo, Rindge, NH (US); Scott Vaughan Plummer, Somerville, MA (US); James Harold Roache, Westford, MA (US); Martin Sendzik, Belmont, MA (US); Ranny Mathew Thomas, Sharon, MA (US); Aimee Richardson Usera, Winchester, MA (US); Anna Vulpetti, Basel (CH); Frederic Zecri, Brookline, MA (US); Liang Zhao, Lexington, MA (US); Thomas Zoller, Andolsheim (FR)

(21) Appl. No.: 18/281,732

(22) PCT Filed: Mar. 10, 2022
(86) PCT No.: PCT/IB2022/000119

§ 371 Date: Sep. 12, 2023

Related U.S. Application Data
(60) Provisional application No. 63/160,498, filed on Mar. 12, 2021.
(60) Provisional application No. 63/160,504, filed on Mar. 12, 2021.

(51) Int. Cl. A61K 47/54; A61K 31/395; A61K 47/10; A61P 35/00

(52) U.S. Cl. CPC A61K 47/542; A61K 31/395; A61K 47/10; A61P 35/00

(57) ABSTRACT

Described herein are fatty acid-bifunctional degrader compounds, their various targets, their preparation, pharmaceutical compositions comprising them, and their use in the treatment of conditions, diseases, and disorders mediated by various target proteins.

### CLAIM OF PRIORITY

This application claims the benefit of priority to U.S. Provisional Application Nos. 63/160,498 filed Mar. 12, 2021 and 63/160,504 filed Mar. 12, 2021, each of which is incorporated by reference herein in its entirety.

### FIELD OF THE DISCLOSURE

Described herein are fatty acid-bifunctional degrader conjugates, their various targets, their preparation, pharmaceutical compositions comprising them, and their use in the treatment of conditions, diseases, and disorders mediated by various target proteins.

### BACKGROUND

Proteasome-mediated degradation of unneeded or damaged proteins plays an important role in maintaining regular cellular functions, such as cell survival, proliferation and growth. In particular, the Ubiquitin-Proteasome Pathway (UPP) is central to multiple cellular processes, and if defective or imbalanced, it leads to pathogenesis of a variety of diseases. The covalent attachment of ubiquitin to specific protein substrates is achieved through the action of E3 ubiquitin ligases. These ligases comprise over 500 different proteins and are categorized into multiple classes defined by the structural element of their E3 functional activity.

The principle of induced degradation of protein targets as a potential therapeutic approach has been described by Crews, J. Med, Chem. 61(2): 403-404 (2018) and references cited therein.

One problem with many of these bifunctional protein degraders is that they suffer from high clearance. High clearance of therapeutic agents is inconvenient in cases where it is desired to maintain a high concentration of the agent over a prolonged period of time at the target site (e.g. in the tumor). Discomfort in administration and high costs, e.g., with frequent administration by injection, are two reasons why therapeutic agents with attractive bioactivity profiles may not ultimately be developed as drug candidates.

Thus, there is a need for selective target protein degraders with improved biological properties for in vivo target validation and as therapeutics. There is a need to provide bifunctional protein degraders in a modified form to provide prolonged exposure thereby resulting in prolonged biological activity.

### SUMMARY

In one aspect, the disclosure provides a conjugate of Formula (I):

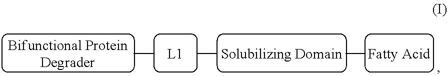

or a pharmaceutically acceptable salt, hydrate, solvate, prodrug, stereoisomer, or tautomer thereof, wherein
(i) a bifunctional protein degrader comprises a bifunctional compound capable of binding to each of a target protein and a ligase independently;
(ii) L1 comprises a cleavable linker;
(iii) optionally, a solubilizing domain comprises a heteroalkylene and is soluble in aqueous solution; and
(iv) a fatty acid comprises a fatty acid capable of binding to a protein.

In another aspect, the disclosure provides a conjugate of Formula (I′):

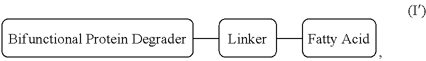

or a pharmaceutically acceptable salt, hydrate, solvate, prodrug, stereoisomer, or tautomer thereof, wherein
(i) a Bifunctional Protein Degrader is a Bruton's tyrosine kinase (BTK) Degrader capable of degrading BTK; and
(ii) a Linker is absent or L⁴, wherein L⁴ is a group that is cleavable to allow release of the Bifunctional Protein Degrader, and that covalently links the Bifunctional Protein Degrader to a Fatty Acid.

In some embodiments, the conjugate of Formula (I′) has a Formula (I′a):

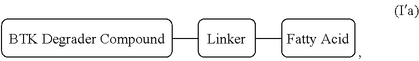

or a pharmaceutically acceptable salt, hydrate, solvate, prodrug, stereoisomer, or tautomer thereof.

In an embodiment, L⁴ comprises L1 and, optionally, a solubilizing domain.

Such conjugates have been observed to advantageously result in sustained exposure of the degrader as well as higher exposure of the degrader in the tumor, whilst having similar or lower exposure in other organs such as the blood, liver, spleen, kidney and heart.

In an embodiment, the Fatty Acid and Solubilizing Domain have Formula SD-FA-I:

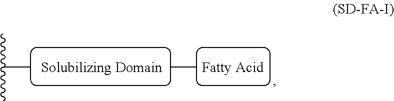

wherein the Solubilizing Domain comprises a heteroalkylene and is soluble in aqueous solution; the Fatty Acid comprises a fatty acid capable of binding to a protein; and

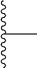

denotes the point of attachment to the bifunctional protein degrader via the linker (L1).

In an embodiment, the linker (L1), Solubilizing Domain, and Fatty Acid have Formula L1-SD-FA-I:

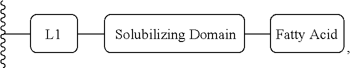

wherein the Solubilizing Domain comprises a heteroalkylene and is soluble in aqueous solution; the Fatty Acid comprises a fatty acid capable of binding to a protein; L1 comprises a cleavable linker; and

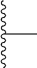

denotes the point of attachment to the bifunctional protein degrader.

In an embodiment, the linker, Solubilizing Domain, and Fatty Acid have Formula L1-SD-FA-II:

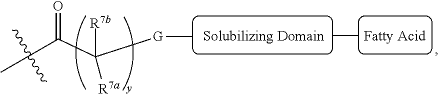

wherein the Solubilizing Domain comprises heteroalkylene and is soluble in aqueous solution; the Fatty Acid comprises a fatty acid capable of binding to a protein; the variables G, R^{7a}, R^{7b}, and y are as defined herein; and

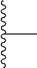

denotes the point of attachment to the bifunctional protein degrader.

In an embodiment, the linker, Fatty Acid and Solubilizing Domain have Formula L1-SD-FA-III(a) and L1-SD-FA-III(b):

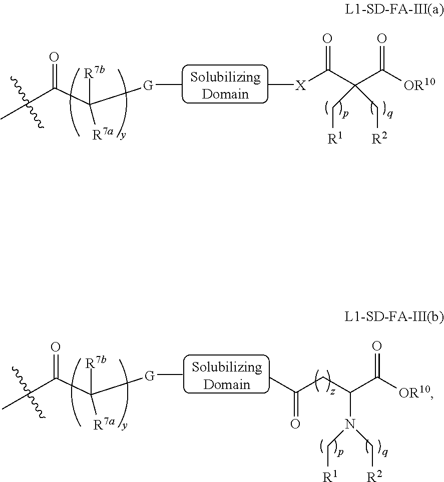

wherein the Solubilizing Domain comprises a heteroalkylene and is soluble in aqueous solution; the variables G, R^{7a}, R^{7b}, R¹, R², R¹⁰, p, q, y, and z are as defined below; and

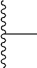

denotes the point of attachment to the bifunctional protein degrader.

In an embodiment, the bifunctional protein degrader has the structure of Formula (I-a):

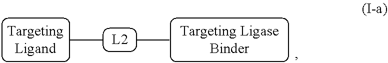

wherein (i) a targeting ligand comprises an entity capable of binding to a target protein; (ii) L2 is a linker; and (iii) a targeting ligase binder comprises an entity capable of binding a ligase.

In an embodiment, the targeting ligase binder has the structure of Formula (TLB-I):

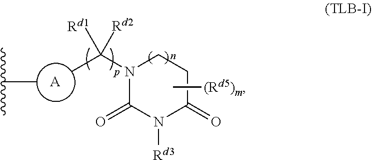

or a pharmaceutically acceptable salt, hydrate, solvate, prodrug, stereoisomer, or tautomer thereof, wherein

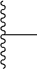

denotes the point of attachment to L2 in Formula (I-a); Ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; m is 1 or 2; n is 1 or 2; and p is 0, 1, or 2.

In an embodiment, n is 1. In an embodiment, p is 1. In an embodiment, R^{d3} is H. In an embodiment, R^{d3} is —CH₂OP(O)(OR^{p})₂.

In an embodiment, ring A is a 5-membered nitrogen-containing heteroaryl or a 6-membered nitrogen-containing heteroaryl (e.g., a nitrogen-containing heteroaryl). In an embodiment, ring A is a 5-membered heteroaryl. In an embodiment, A is a 5-membered nitrogen-containing heteroaryl. In an embodiment, A is a 6-membered heteroaryl. In an embodiment, ring A is a 6-membered nitrogen-containing heteroaryl. In an embodiment, ring A is selected from the group consisting of phenyl, pyridyl, pyridonyl, pyrazinyl, thiophenyl, pyrazolyl, imidazolyl, and pyrrolyl. In an embodiment, ring A is pyridyl or pyridonyl. In an embodiment, R^{d4} is hydroxyl or C₁₋₆ alkoxyl.

In an embodiment, the targeting ligase binder has a structure selected from the group consisting of Formulas (TLB-I-i), (TLB-I-ii), and (TLB-I-iii):

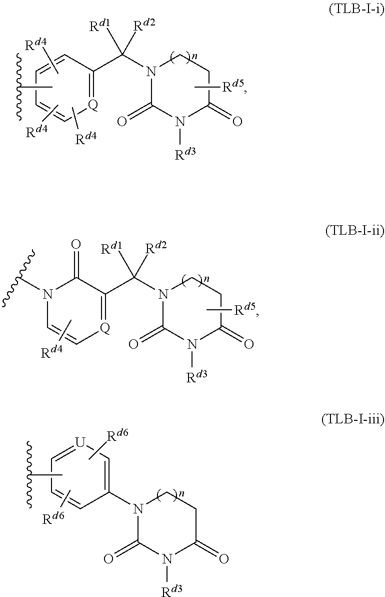

or a pharmaceutically acceptable salt, hydrate, solvate, prodrug, stereoisomer, or tautomer thereof, wherein

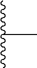

denotes the point of attachment to L2 in Formula (I-a); Q is N or CR^{d4}; U is —CR^{d6} or N; R^{d1} and R^{d2} are each independently selected from the group consisting of H, C₁₋₆ alkyl, C₁₋₆ haloalkyl, and C₁₋₆ heteroalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; R^{d5} is selected from the group consisting of H, C₁₋₆ alkyl, halogen, C₁₋₆ haloalkyl, and C₁₋₆ heteroalkyl; each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; R^{p} is H or C₁₋₆ alkyl; and n is 1 or 2.

In an embodiment, n is 1. In an embodiment, R^{d3} is H. In an embodiment, R^{d3} is —CH₂OP(O)(OR^{p})₂.

In an embodiment, R^{d4} is hydroxyl or C₁₋₆ alkoxyl.

In an embodiment, R^{d1} is H. In an embodiment, R^{d2} is H. In an embodiment, R^{d1} and R^{d2} are both H.

In an embodiment, n is 1. In an embodiment, n is 2. In an embodiment, U is N. In an embodiment, U is —CR^{d6}. In an embodiment, each R^{d6} is independently selected from the group consisting of H, methyl, halogen, methoxy, and methoxymethyl. In an embodiment, R^{d6} is H. In an embodiment, R^{d6} is methyl. In an embodiment, R^{d6} is halogen. In an embodiment, R^{d6} is methoxy.

In an embodiment, the L2 has a structure of Formula (L-I):

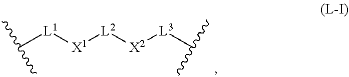

or a pharmaceutically acceptable salt, hydrate, solvate, prodrug, stereoisomer, or tautomer thereof,
   wherein L¹ is selected from the group consisting of a bond, O, NR′, C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand in Formula (I-a); X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, O, NR′, C(O), C₁₋₆ alkylene, C₁₋₆ heteroalkylene, and *C(O)NR′—C₁₋₆ alkylene, wherein * denotes the point of attachment of L² to X²; or X¹-L²-X² form a spiroheterocyclyl, which is substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from a bond, C₁₋₆ alkylene, C₂₋₆ alkenylene, C₂₋₆ alkynylene, C₁₋₆ heteroalkylene, C(O), S(O)₂, O, NR′, *C(O)—C₁₋₉ alkylene, *C(O)—C₁₋₆ alkylene-O, and *C(O)—C₁₋₉ heteroalkylene, wherein * denotes the point of attachment of L³ to X² in (L-I); wherein no more than 2 of L¹, X¹, X², L², and L³ can simultaneously be a bond; and R′ is hydrogen or C₁₋₆ alkyl.

In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl.

In an embodiment, one of X¹ and X² is a bond, and the other is a heterocyclyl. In an embodiment, X¹ and X² are each independently selected from piperidinyl and piperazinyl. In an embodiment, X¹ and X² are both piperidinyl. In an embodiment, —X¹-L²-X²— is:

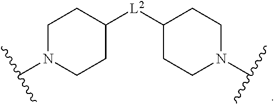

In an embodiment, L2 is a compound having the following formula:

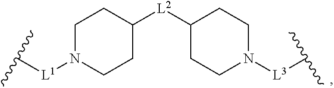

or a pharmaceutically acceptable salt, hydrate, solvate, prodrug, stereoisomer, or tautomer thereof.

In an embodiment, —X¹-L²-X²— forms a spiroheterocyclyl having the structure,

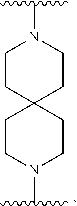 [SMILES: *N1CCC2(CC1)CCN(*)CC2]

substituted with 0-4 occurrences of R^{b}, wherein each R^{b} is independently selected from C₁₋₆ alkyl, C₁₋₆ alkoxyl, C₁₋₆ hydroxyalkyl, and halogen.

In an embodiment, —X¹-L²-X²— forms a spiroheterocyclyl having the structure,

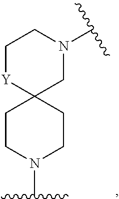

substituted with 0-4 occurrences of R^{b}, wherein Y is selected from CH₂, oxygen, and nitrogen; and each R^{b} is independently selected from C₁₋₆ alkyl, C₁₋₆ alkoxyl, C₁₋₆ hydroxyalkyl, and halogen.

In an embodiment, X¹ and X² are each a bond. In an embodiment, L³ is independently selected from the group consisting of —C(O)—, C₂₋₆ alkynylene, or C₁₋₆ heteroalkylene; and L¹ is —C(O)—, C₁₋₈ alkylene, C₁₋₈ heteroalkylene, and *C₁₋₆ alkylene-C(O). In an embodiment, L³ is selected from the group consisting of —C(O)—, —O—C₁₋₆ alkylene, C₂₋₆ alkynylene, and C₁₋₆ heteroalkylene; and L¹ is C₁₋₈ alkylene or C₁₋₈ heteroalkylene. In an embodiment, L³ is —C(O)— or C₁₋₆ heteroalkylene; and L¹ is C₁₋₈ alkylene or C₁₋₈ heteroalkylene. In an embodiment, L³ is a bond or —O—; and L¹ is —C(O)— or C₁₋₈ heteroalkylene. In an embodiment, L³ is selected from the group consisting of —O—, —C(O)—, —S(O)₂—, and C₁₋₆ heteroalkylene; and L¹ is C₁₋₈ alkylene or C₁₋₈ heteroalkylene. In an embodiment, L² is —C(O)—, —NR′—, or C₁₋₆ alkylene. In an embodiment, L² is —C(O)—, —O—, or C₁₋₆ alkylene. In an embodiment, L² is C₁₋₆ alkylene. In an embodiment, L² is selected from the group consisting of —C(O)—, C₁₋₆ alkylene, C₁₋₆ heteroalkylene, and *C(O)NR′—C₁₋₆ alkylene. In an embodiment, Y is CH₂, CH(C₁₋₃ alkyl), C(C₁₋₃ alkyl)₂, oxygen, NH, or N(C₁₋₃ alkyl).

In an embodiment, the targeting ligase binder and L2 have a structure of Formula (TLB-L2-I):

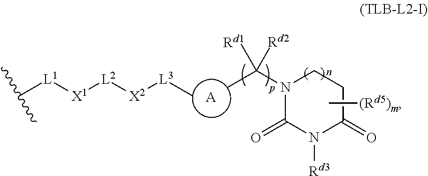

or a pharmaceutically acceptable salt, hydrate, solvate, prodrug, stereoisomer, or tautomer thereof, wherein

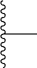

denotes the point of attachment to the targeting ligand in Formula (I-a); L¹ is selected from the group consisting of a bond, —O—, —NR′—, —C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O), —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L2-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2.

In an embodiment, ring A is a 5-membered nitrogen-containing heteroaryl or a 6-membered nitrogen-containing heteroaryl (e.g., a nitrogen-containing heteroaryl). In an embodiment, ring A is a 5-membered heteroaryl. In an embodiment, A is a 5-membered nitrogen-containing heteroaryl. In an embodiment, A is a 6-membered heteroaryl. In an embodiment, ring A is a 6-membered nitrogen-containing heteroaryl. In an embodiment, ring A is selected from the group consisting of phenyl, pyridyl, pyridonyl, pyrazinyl, thiophenyl, pyrazolyl, imidazolyl, and pyrrolyl. In an embodiment, ring A is pyridyl or pyridonyl. In an embodiment, R^{d4} is hydroxyl or C₁₋₆ alkoxyl.

In an embodiment, the targeting ligase binder and L2 have a structure selected from the group consisting of Formulas (TLB-L2-I-i), (TLB-L2-I-ii), and (TLB-L2-I-iii):

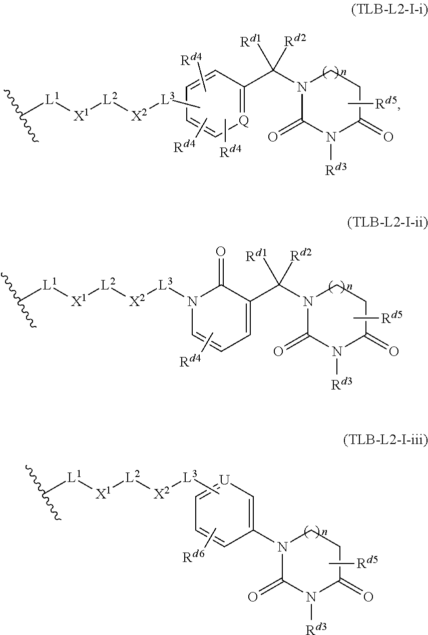

or a pharmaceutically acceptable salt, hydrate, solvate, prodrug, stereoisomer, or tautomer thereof,
wherein

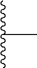

denotes the point of attachment to the targeting ligand in Formula (I-a); L¹ is selected from the group consisting of a bond, —O—, —NR′—, —C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or X¹-L²-X² form a spiroheterocyclyl substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O), —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-II); wherein no more than 2 of L¹, X¹, X², L², and L³ can simultaneously be a bond; Q is N or CR^{d4}; U is —CR^{d6} or N; R^{d1} and R^{d2} are each independently selected from the group consisting of H, C₁₋₆ alkyl, C₁₋₆ haloalkyl, and C₁₋₆ heteroalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl; R^{d5} is selected from the group consisting of H, C₁₋₆ alkyl, halogen, C₁₋₆ haloalkyl, and C₁₋₆ heteroalkyl; each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; R^{p} is H or C₁₋₆ alkyl; and n is 1 or 2.

In an embodiment, n is 1. In an embodiment, R^{d3} is H. In an embodiment, R^{d3} is —CH₂OP(O)(OR^{p})₂.

In an embodiment, n is 2. In an embodiment, L³ is selected from the group consisting of —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl.

In another embodiment, the targeting ligase binder and L2, or a pharmaceutically acceptable salt, hydrate, solvate, prodrug, stereoisomer, or tautomer thereof, have a structure selected from:

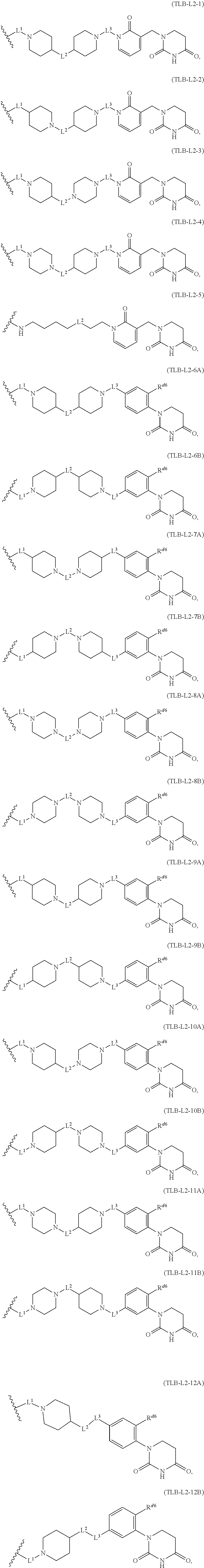

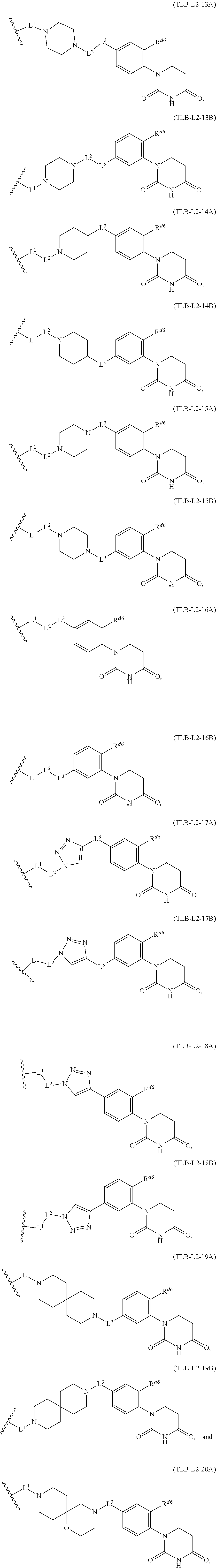

or a pharmaceutically acceptable salt, hydrate, solvate, prodrug, stereoisomer, or tautomer thereof, wherein each of L¹, L², L³, and R^{d6} is as defined herein, and

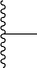

denotes the point of attachment to the targeting ligand in Formula (I-a).

In another aspect, the bifunctional protein degrader (e.g., of Formula (I-a)) has a structure of Formula (BFD-I):

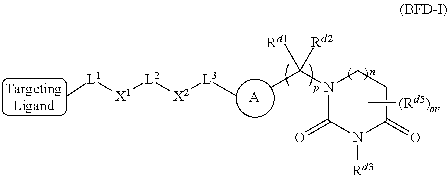

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand;
X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BFD-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2, wherein the targeting ligand is a group capable of binding to a target protein.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered heteroaryl. In an embodiment, ring A is a 5-membered nitrogen-containing heteroaryl. In an embodiment, ring A is a 6-membered heteroaryl. In an embodiment, ring A is a 6-membered nitrogen-containing heteroaryl. In an embodiment, ring A is pyridyl.

In an embodiment, n is 1. In an embodiment, n is 2. In an embodiment, R^{d3} is —CH₂OP(O)(OR^{p})₂. In an embodiment, R^{d3} is H.

In another embodiment, the bifunctional protein degrader (e.g, of Formula (I-a)) has a structure selected from the group consisting of Formulas (BFD-1-i), (BFD-I-ii), and (BFD-I-iii):

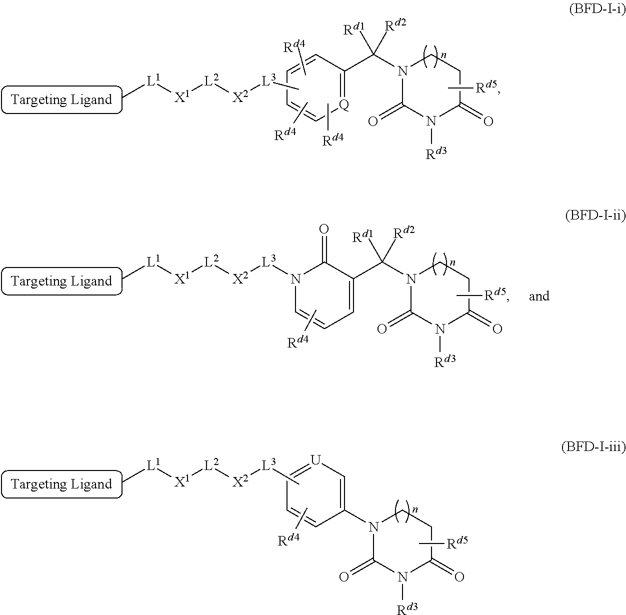

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in any one of Formulas (BFD-1-i), (BFD-I-ii), or (BFD-I-iii); wherein no more than 2 of L¹, X¹, X², L², and L³ can simultaneously be a bond; Q is N or CR^{d4}; U is —CR^{d6} or N; R^{d1} and R^{d2} are each independently selected from the group consisting of H, C₁₋₆ alkyl, C₁₋₆ haloalkyl, and C₁₋₆ heteroalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl; R^{d5} is selected from the group consisting of H, C₁₋₆ alkyl, halogen, C₁₋₆ haloalkyl, and C₁₋₆ heteroalkyl; each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; R^{p} is H or C₁₋₆ alkyl; and n is 1 or 2, wherein the targeting ligand is a group capable of binding to a target protein.

In an embodiment, n is 1. In an embodiment, n is 2. In an embodiment, R^{d3} is —CH₂OP(O)(OR^{p})₂. In an embodiment, R^{d3} is H.

In an embodiment, R^{d3} is H. In an embodiment, —X¹-L²-X²— is:

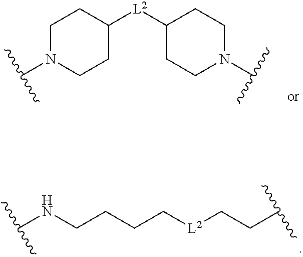

In an embodiment, L¹ is —O— or C₁₋₆ alkylene. In an embodiment, R^{d1} and R^{d2} are both methyl. In an embodiment, R^{d1} and R^{d2} are both H. In an embodiment, R^{d4} is H or C₁₋₃ alkyl. In an embodiment, R^{d5} is H or C₁₋₃ alkyl.

In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl.

In an embodiment, R^{d7} is —CH₂OP(O)(OR^{p})₂. In an embodiment, R^{d7} is H. In an embodiment, U is —CR^{d6}. In an embodiment, R^{d8} is H. In an embodiment, R^{d7} and R^{d8} are each independently H. In an embodiment, R^{d6} is H. In an embodiment, R^{d6} is selected from the group consisting of H, halogen, C₁₋₆ alkyl, and C₁₋₆ alkoxyl. In an embodiment, R^{d6} is selected from the group consisting of H, halogen, C₁₋₆ alkyl, and C₁₋₆ alkoxyl; and R^{d7}, and R^{d8} are each H.

In another embodiment, L¹-X¹-L²-X²-L³ is selected from the group consisting of:

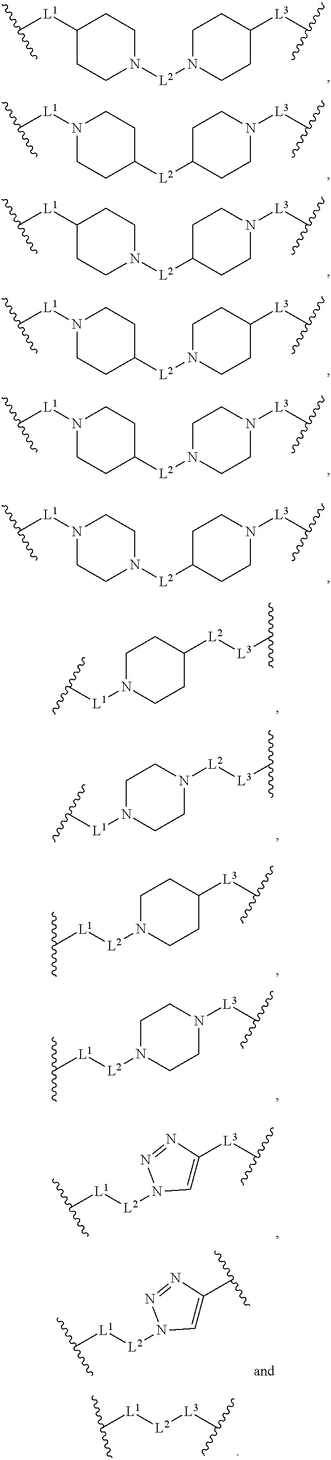

In an embodiment, L³ is selected from the group consisting of a bond, —O—, —C(O)—, —S(O)₂—, C₁₋₆ alkylene, C₂₋₆ alkynylene, and C₁₋₆ heteroalkylene.

In an embodiment, the targeting ligand is a BTK targeting ligand. In an embodiment, the targeting ligand is a BTK targeting ligand of Formula (BTK-I):

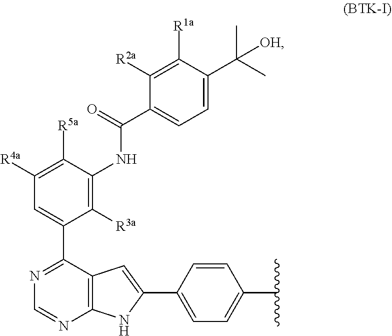

or a pharmaceutically acceptable salt, hydrate, solvate, prodrug, stereoisomer, or tautomer thereof, wherein R^{1a} is H or halo; R^{2a} is halo; R^{3a} is C₁₋₆ alkyl; R^{4a} is halo; and R^{5a} is H or halo.

In another embodiment, the bifunctional protein degrader (e.g., of Formula (I-a)) or the BTK degrader Compound has a structure of Formula (BFD-BTK-I), (BFD-BTK-II), (BFD-BTK-III), (BFD-BTK-I-a), (BFD-BTK-II-a), or (BFD-BTK-III-a):

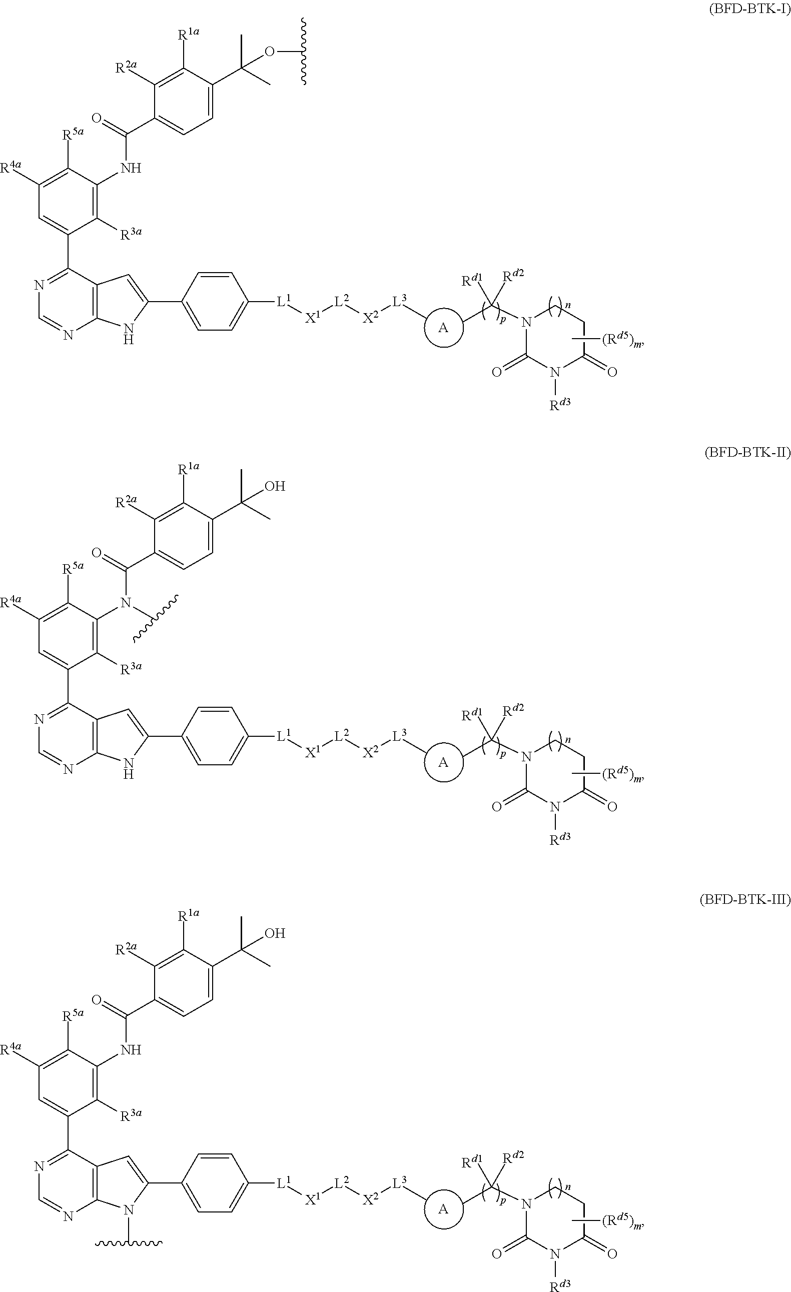

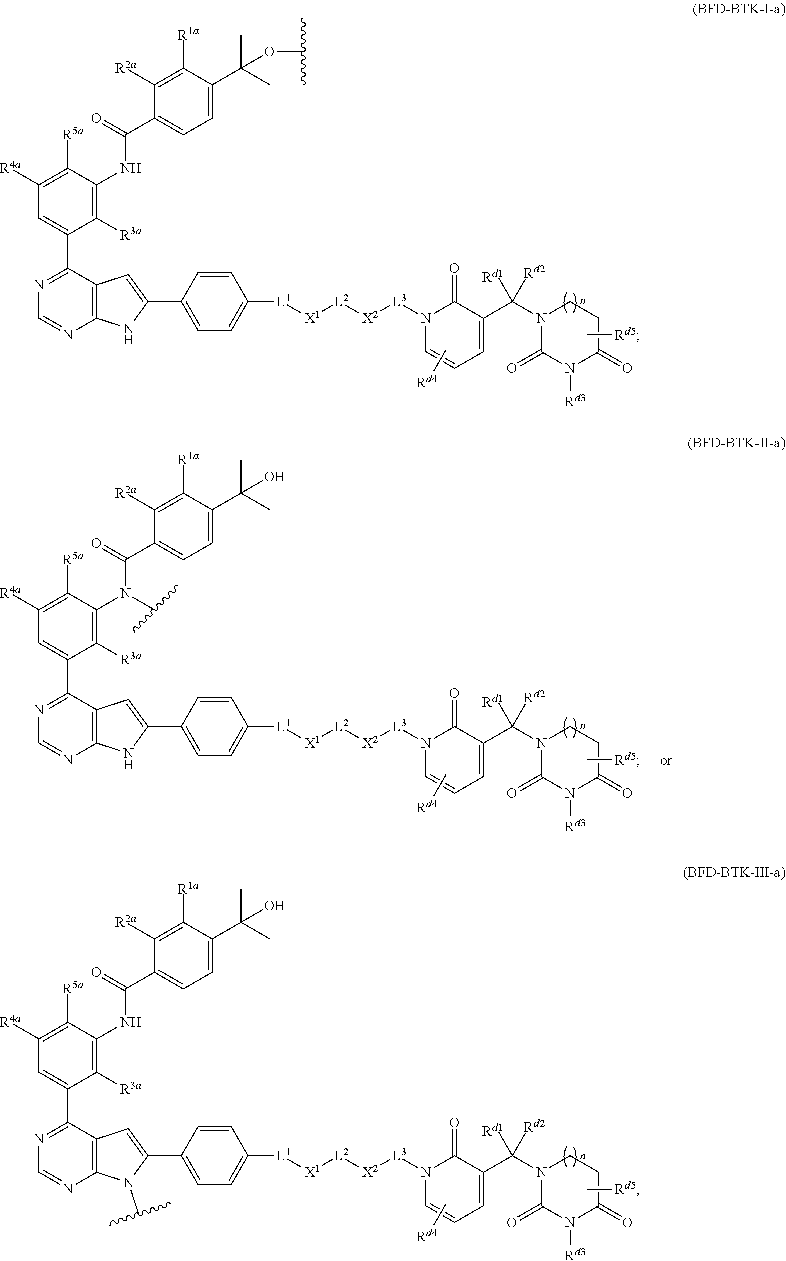

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to X¹; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X²; wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; n is 1 or 2; R^{1a} is H or halo; R^{2a} is H or halo; R^{3a} is C₁₋₆ alkyl; R^{4a} is H or halo; R^{5a} is H or halo; and

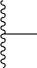

denotes the point of attachment to L1 in Formula (I) or the point of attachment to the Linker L⁴ in Formula (I′) or Fatty Acid when the Linker is absent.

In an embodiment, the bifunctional protein degrader (e.g., of Formula (I-a)) or the BTK degrader Compound has a structure of Formula (BFD-BTK-I), (BFD-BTK-II), or (BFD-BTK-III). In an embodiment, the bifunctional protein degrader (e.g., of Formula (I-a)) or the BTK degrader Compound has a structure of Formula ((BFD-BTK-I-a), (BFD-BTK-II-a), or (BFD-BTK-III-a). The variables are as defined above.

In such BTK Degrader Compounds of Formula (BFD-BTK-II) or (BFD-BTK-III) and (BFD-BTK-II-a) or (BFD-BTK-III-a), the linker and Fatty Acid are as described in relation to compounds of Formula (BFD-BTK-I) and Formula (BFD-BTK-I-a), respectively, except in that the cleavable portion of the linker may also be

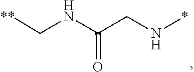

wherein ** indicates the point of attachment to the BTK degrader, and * indicates the point of attachment to the solubilizing portion, when present, of the linker.

In an embodiment, the cleavable portion of the linker is an ester.

In an embodiment, the conjugate of Formula (I′) wherein L⁴ comprises L1 and, optionally, a solubilizing domain. In an embodiment, the conjugate of Formula (I) or (I′) comprises a cleavable linker L1 that connects the bifunctional protein degrader, and when present, the solubilizing domain. In an embodiment, the cleavable linker L1 is covalently linked to the bifunctional protein degrader. In an embodiment, the cleavable linker L1 is covalently linked to the solubilizing domain, when present. In an embodiment, the cleavable linker L1 is covalently linked to both the bifunctional protein degrader and the solubilizing domain, when present.

The cleavable linker L1 may be degraded or hydrolyzed at physiological conditions. In some embodiments, L1 comprises a bond cleavable in a cell (e.g, a cell organelle) or the serum, e.g., of a sample or subject. For example, L1 may be pH sensitive (e.g., acid labile or base labile) or cleaved through the action of an enzyme. In an embodiment, the rate of hydrolysis of L1 is increased by at least 0.5 fold (e.g., at least 1, 1.5, 2, 2.5, 3, 4, 5, 7.5, 10, 12.5, 15, 20, 25, 50, 75, 100, 250, 500, 750, 1000 or more) compared with the rate of hydrolysis of L1 in the absence of an enzyme. In an embodiments, the enzyme is an esterase.

In an embodiment, L1 comprises an ester, phosphate, disulfide, thiol, hydrazone, ether, or amide. In an embodiment, L1 comprises an ester. In an embodiment, L1 has the structure of Formula (L1-I) or Formula (L1-II):

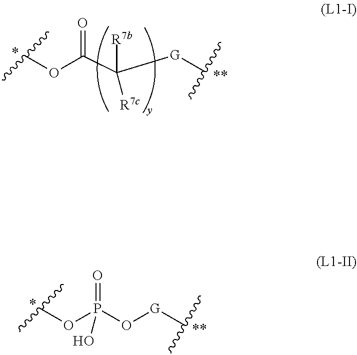

or a pharmaceutically acceptable salt, hydrate, solvate, prodrug, stereoisomer, or tautomer thereof, wherein each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, —NR′— wherein R′ is H, C₁₋₆ alkyl, or
—(CH₂)₁₋₂—C(O)₂H, one or more natural or unnatural amino acids, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; and each “*” and “**” independently denote the point of attachment to the bifunctional protein degrader or solubilizing domain, when present, or the fatty acid in Formula (I) or (I′).

In one embodiment, each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, —NR′— wherein R′ is H, C₁₋₆ alkyl, or —(CH₂)₁₋₂—C(O)₂H, 1 to 5 natural or unnatural amino acids, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; and each “*” and “**” independently denote the point of attachment to the bifunctional protein degrader or solubilizing domain in L1 in Formula (I) or (I′).

In another embodiment, each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; and each “*” and “**” independently denote the point of attachment to the bifunctional protein degrader or solubilizing domain, when present, in L1 in Formula (I) or (I′). In an embodiment, when the solubilizing domain is not present, then each “*” and “**” independently denote the point of attachment to the bifunctional protein degrader and the fatty acid.

In an embodiment, L1 is selected from the group consisting of:

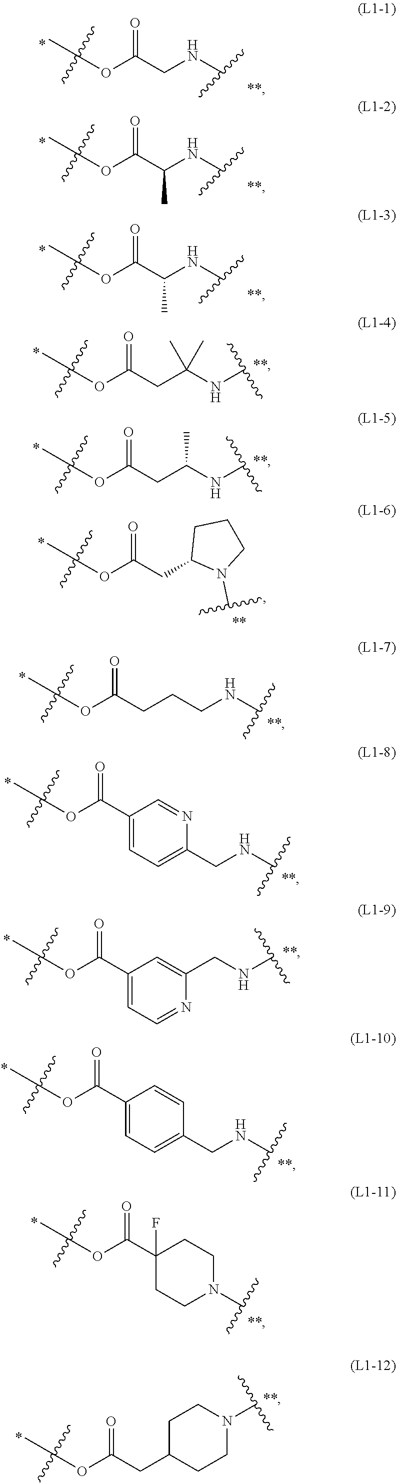

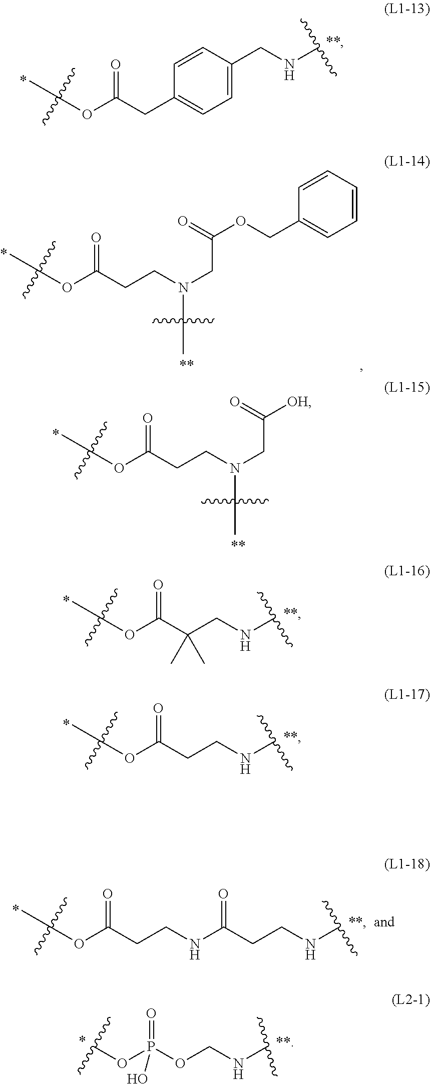

In an embodiment, the bifunctional protein degrader and L1 have the structure of Formula (BFD-L1-I):

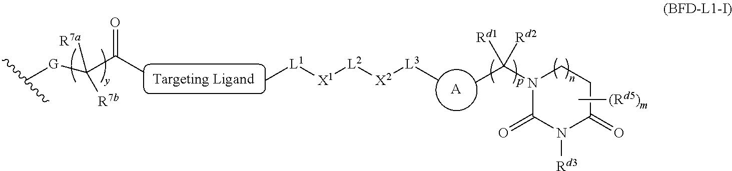

or a pharmaceutically acceptable salt, hydrate, solvate, prodrug, stereoisomer, or tautomer thereof, wherein each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, —NR′— wherein R′ is H, C₁₋₆ alkyl, or —(CH₂)₁₋₂—C(O)₂H, 1 to 5 natural or unnatural amino acids, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BFD-L1-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2, wherein the targeting ligand is a group capable of binding to a target protein, and

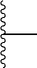

denotes the point of attachment to the solubilizing domain, when present, in Formula (I) or (I′).

In another embodiment, the bifunctional protein degrader and L1 have the structure of Formula (BFD-L1-II):

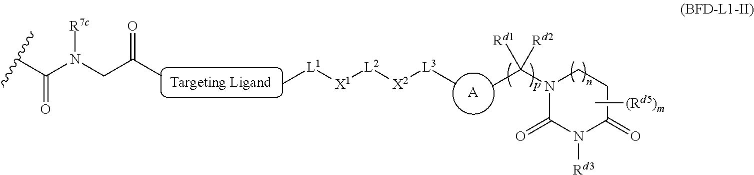

or a pharmaceutically acceptable salt, hydrate, solvate, prodrug, stereoisomer, or tautomer thereof, wherein R^{7c} is H or C₁₋₆ alkyl; L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-0, and *C(O)—C₁₋₉ heteroalkylene, wherein * denotes the point of attachment of L³ to X² in Formula (BFD-L1-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2, wherein the targeting ligand is a group capable of binding to a target protein, and

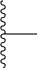

denotes the point of attachment to the solubilizing domain, when present, in Formula (I) or (I′).

In an embodiment, the conjugate of Formula (I) or (I′) comprises a solubilizing domain, when present, that comprises a water-soluble monomer or polymer. In an embodiment, the solubilizing domain, when present, increases one or more of amphiphilicity, hydrophilicity, water-solubility, pH sensitivity, or stability of the conjugate of Formula (I) or (I′), e.g., compared to a conjugate that does not comprise the solubilizing domain.

In an embodiment, the solubilizing domain, when present, comprises a polyalkylene or polyheteroalkylene moiety. In an embodiment, the solubilizing domain, when present, comprises a polyethylene glycol (PEG), a polyethylene oxide (PEO), a polypropylene glycol (PPG), a polyglycerol (PG), a poloxamine (POX), a polybutylene oxide (PBO), polylactic acid (PLA), polyglycolic acid (PGA), poly(lactic-co-glycolic acid) (PLGA), polycaprolactone (PCL), polydioxanone (PDO), a polyanhydride, a polyacrylide, a polyvinyl, or a polyorthoester. In an embodiment, the solubilizing domain, when present, comprises a polyethylene glycol (PEG).

In an embodiment, the solubilizing domain, when present, is between 100 Da and 20,000 Da in size. In an embodiment, the solubilizing domain, when present, is between 200 Da and 1,000 Da in size. In an embodiment, the solubilizing domain, when present, comprises a PEG between 100 Da and 20,000 Da in size. In an embodiment, the solubilizing domain, when present, comprises a PEG between 200 Da and 1,000 Da in size.

In an embodiment, the solubilizing domain, when present, comprises a PEG2, PEG3, PEG4, PEG5, PEG6, PEG7, PEG8, PEG9, PEG10, PEG11, PEG12, PEG13, PEG14, PEG15, PEG16, PEG17, PEG18, PEG19, PEG20, PEG21, PEG22, PEG23, PEG24, PEG25, PEG26, PEG27, PEG28, PEG29, or PEG30. In an embodiment, the solubilizing domain, when present, is selected from PEG2, PEG4, PEG6, PEG8, PEG10, PEG12, PEG16, PEG18, and PEG24.

In an embodiment, the solubilizing domain, when present, has a structure selected from the group consisting of Formulas (SD-I), (SD-II), and (SD-III):

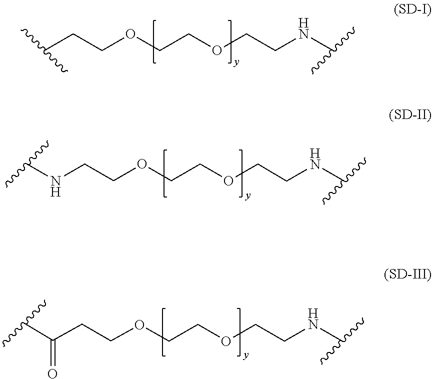

wherein y is an integer between 0 to 35; and

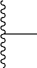

denotes the points of attachment to L1 and the fatty acid in Formula (I) or (I′).

In an embodiment, y is 5 to 30, e.g., 6 to 20, e.g., 7 to 15, e.g., 9 to 13, or e.g., 11.

In an embodiment, the solubilizing domain, when present, has the structure of Formula (SD-I):

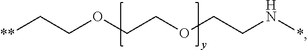

wherein * indicates the point of attachment to the fatty acid, ** indicates the point of attachment to L1, and y is 11.

In an embodiment, the bifunctional protein degrader, L1, and solubilizing domain have the structure of Formula (BFD-L1-SD-I):

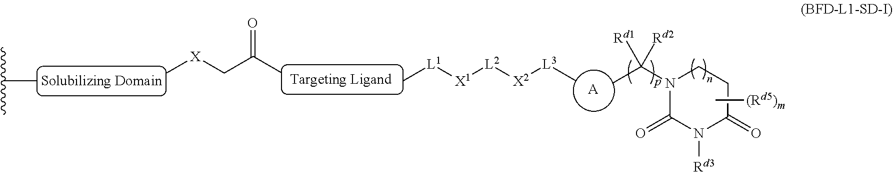

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-0, and *C(O)—C₁₋₉ heteroalkylene, wherein * denotes the point of attachment of L³ to X² in Formula (BFD-L1-SD-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2; X is O, S, C(R^{7a})(R^{7b}), N(R^{7c})C(O), C₁₋₆ alkyl, C₁₋₆ heteroalkyl, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; and

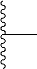

denotes the point of attachment to the fatty acid in Formula (I) or (I′).

In an embodiment, the bifunctional protein degrader, L1, and solubilizing domain, when present, have the structure of Formula (BFD-L1-SD-I):

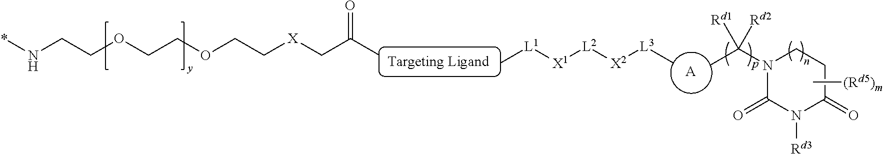

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-0, and *C(O)—C₁₋₉ heteroalkylene, wherein * denotes the point of attachment of L³ to X² in Formula (BFD-L1-SD-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2; X is O, S, C(R^{7a})(R^{7b}), N(R^{7c})C(O), C₁₋₆ alkyl, C₁₋₆ heteroalkyl, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is an integer between 0 and 35; and * indicates the point of attachment to the fatty acid in Formula (I) or (I′).

In an embodiment, the conjugate of Formula (I) or (I′) comprises a fatty acid capable of binding to a protein (e.g., a soluble or membrane protein, e.g., albumin). In some embodiments, the fatty acid improves the plasma stability half-life, e.g., compared to a compound that does not comprise a fatty acid.

In an embodiment, the fatty acid has a structure selected from the group consisting of Formula (FA-1), Formula (FA-2), and Formula (FA-3):

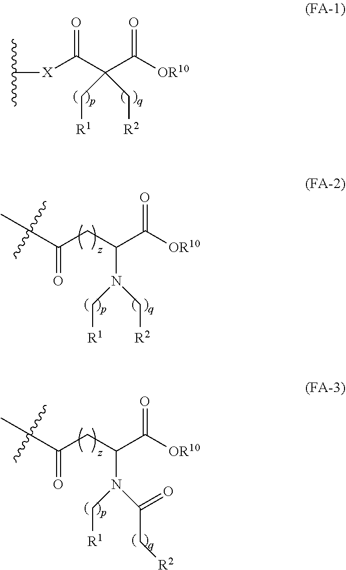

or a pharmaceutically acceptable salt, hydrate, solvate, prodrug, stereoisomer, or tautomer thereof, wherein X is 0 or N(R³); p and q are each an integer independently selected from 5 to 30; z is an integer selected from 0 to 5; R¹ and R² are each independently selected from CH₃, OR¹⁰, C(O)OR¹⁰, and P(O)(OR¹⁰)₂, R³ and R¹⁰ are each independently H or C₁₋₆ alkyl; and

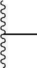

denotes the point of attachment to the solubilizing domain, when present, in Formula (I) or (I′).

In an embodiment, the fatty acid has a structure of Formula (FA-1).

In an embodiment, the fatty acid of Formula (FA-1) has a structure selected from Formula (FA-1a) and (FA-1b):

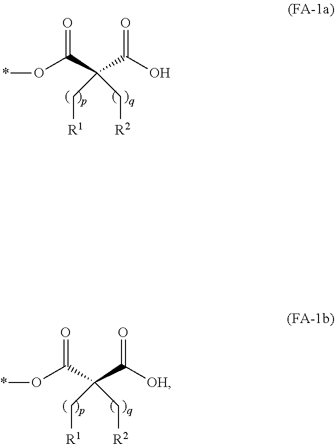

or a pharmaceutically acceptable salt, hydrate, solvate, prodrug, stereoisomer, or tautomer thereof, wherein p and q are each an integer independently selected from 5 to 30; R¹ and R² are each independently selected from CH₃, OR¹⁰, C(O)OR¹⁰, and P(O)(OR¹⁰)₂; R¹⁰ are each independently H or C₁₋₆ alkyl; and * denotes the point of attachment to the solubilizing domain, when present, in Formula (I) or (I′).

In an embodiment, the fatty acid has a structure of Formula (FA-2). In an embodiment, the fatty acid of Formula (FA-2) has a structure selected from Formula (FA-2a) and (FA-2b):

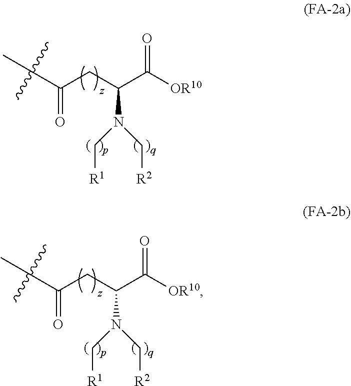

or a pharmaceutically acceptable salt, hydrate, solvate, prodrug, stereoisomer, or tautomer thereof, wherein p and q are each an integer independently selected from 5 to 30; z is an integer selected from 0 to 5; R¹ and R² are each independently selected from CH₃, OR¹⁰, C(O)OR¹⁰, and P(O)(OR¹⁰)₂; R¹⁰ is H or C₁₋₆ alkyl; and

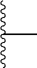

denotes the point of attachment to the solubilizing domain, when present, in Formula (I) or (I′).

In an embodiment, the fatty acid has a structure of Formula (FA-3). In an embodiment, the fatty acid of Formula (FA-3) has a structure selected from Formula (FA-3a) and (FA-3b):

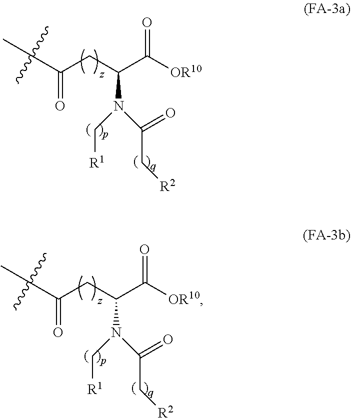

or a pharmaceutically acceptable salt, hydrate, solvate, prodrug, stereoisomer, or tautomer thereof, wherein p and q are each an integer independently selected from 5 to 30; z is an integer selected from 0 to 5; R¹ and R² are each independently selected from CH₃, OR¹⁰, C(O)OR¹⁰, and P(O)(OR¹⁰)₂; R¹⁰ is H or C₁₋₆ alkyl; and

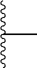

denotes the point of attachment to the solubilizing domain, when present, in Formula (I) or (I′).

Another embodiment is a pharmaceutical composition comprising a compound described herein, or a pharmaceutically acceptable salt, hydrate, solvate, prodrug, stereoisomer, or tautomer thereof, and a pharmaceutically acceptable carrier.

Another embodiment is a pharmaceutical combination comprising a compound described herein, or a pharmaceutically acceptable salt, hydrate, solvate, prodrug, stereoisomer, or tautomer thereof, and one or more additional therapeutic agent(s).

Another embodiment is a method for inducing degradation of a target protein in a subject in need thereof, the method comprising administering to the subject a therapeutically effective amount of a compound described herein, or a pharmaceutically acceptable salt, hydrate, solvate, prodrug, stereoisomer, or tautomer thereof.

Another embodiment is a method of inhibiting, reducing, or eliminating the activity of a target protein, the method comprising administering to the subject a compound described herein, or a pharmaceutically acceptable salt, hydrate, solvate, prodrug, stereoisomer, or tautomer thereof.

In an embodiment, inhibiting, reducing, or eliminating the activity of a target protein comprises recruiting a ligase with the targeting ligase binder, e.g., a targeting ligase binder described herein, of the bifunctional protein degrader, e.g., a bifunctional protein degrader described herein, forming a ternary complex of the target protein, fatty acid-bifunctional degrader conjugate, and the ligase, to thereby inhibit, reduce or eliminate the activity of the Target Protein.

In an embodiment, the Target Protein is selected from Table 1:

TABLE 1
Exemplary Target Proteins

| Target Code | Target Name |
|---|---|
| a5b1 | Integrin a5b1 |
| AAK1 | Adaptor protein complex 2-associated protein kinase 1 |
| ABL1 | Abelson Tyrosine-Protein Kinase 1 including T3141 |
| ACAT2 | Acetyl-CoA Acetyltransferase 2 |
| ADAR | adenosine deaminase RNA specific |
| ADORA2A | adenosine A2a receptor |
| AHR | Aryl hydrocarbon receptor |
| Akt | Protein Kinase B |
| ALK | ALK receptor tyrosine kinase |
| AR | Androgen hormone receptor including |
| ARAF | A-Raf proto-oncogene, serine/threonine kinase |
| ASC | Apoptosis-Associated Speck-Like |
| ATP4 | ATP Synthase Subunit 4 |
| AURKA | Aurora Kinase A |
| AURKB | Aurora Kinase B |
| BACH1 | BTB and CNC homology 1 |
| BACH2 | BTB and CNC homology 2 |
| BCL 2 | BCL2 apoptosis regulator |
| BCL11A | BAF Chromatin Remodeling Complex Subunit BCL11A |
| BCL2L1 | BCL2 like 1 |
| BCR-ABL | BCR-ABL protein |
| BKV JCV | Human Polyomaviruses - capsid |
| BLK | B-lymphoid tyrosine kinase |
| BRAF | B-Raf proto-oncogene, serine/threonine kinase |
| BRD2 | Bromodomain containing protein 2 |
| BRD3 | Bromodomain containing protein 3 |
| BRD4 | Bromodomain containing protein 4 |
| BRD5 | Bromodomain containing protein 5 |
| BRD7 | Bromodomain containing protein 7 |
| BRD8 | Bromodomain containing protein 8 |
| BRD9 | Bromodomain containing protein 9 |
| BRDT | bromodomain testis associated protein |
| BTK | Bruton's tyrosine kinase |
| BTK mutant | various Bruton's tyrosine kinase mutations |
| BTKC481S | mutant Bruton's tyrosine kinase |
| BTLA | B and T lymphocyte associated |
| CCR7 | C-C motif chemokine receptor 7 |
| CD274 | CD274 molecule |
| CD47 | CD47 molecule |
| CDK12 | Cyclin Dependent Kinase 12 |
| CDK13 | Cyclin Dependent Kinase 13 |
| CDK17 | Cyclin Dependent Kinase 17 |
| CDK2 | Cyclin Dependent Kinase 2 |
| CDK4 | Cyclin Dependent Kinase 4 |
| CDK5 | Cyclin Dependent Kinase 5 |
| CDK6 | Cyclin Dependent Kinase 6 |
| CDK7 | Cyclin Dependent Kinase 7 |
| CDK9 | Cyclin Dependent Kinase 9 |
| CDPK1 | Calcium Dependent Protein Kinase 1 |
| CDPK4 | Calcium Dependent Protein Kinase 4 |
| CDX2 | caudal type homeobox 2 |
| CEBPA | CCAAT enhancer binding protein alpha |
| cGAS | Cyclic GMP-AMP Synthase ATI |
| c-Met | Met tyrosine-protein kinase or hepatocyte growth factor receptor |
| | (HGFR) |
| CRABP-I/II | Cellular retinoic acid-binding protein 1/2 |
| CREBBP | CREB binding protein |
| CSF1 | colony stimulating factor 1 |
| CSK | C-terminal Src kinase |
| CTLA4 | cytotoxic T-lymphocyte associated protein 4 |
| CTNNB1 | catenin beta 1 |
| DAPK1 | Death-associated protein kinase 1 |
| DDX58 | DExD/H-Box Helicase 58 |
| DHFR | Dihydrofolate reductase |
| DHODH | Dihydroorotate dehydrogenase |
| DNMT3A | DNA methyltransferase 3 alpha, WT and variant R882H, C, or P |
| DOT1L | DOT1L like histone lysine methyltransferase |
| DXR | 1-deoxy-D-xylulose 5-phosphate reductoisomerase |
| EBF1 | EBF transcription factor 1 |
| EGFR | Epidermal growth factor receptor |
| EGFR L858R | Epidermal growth factor receptor L859→R mutation |
| EGFR Δ Exon 19 | Epidermal growth factor receptor exon 19 deletion |
| ENTPD2 | ectonucleoside triphosphate diphosphohydrolase 2 |
| EP300 | E1A binding protein p300 |
| EPAS1 | Endothelial PAS domain protein 1 |
| ER | Estrogen receptor |
| ER | Estrogen receptor mutant including ESR Y573S/C/N; ESR D538G |
| ERRα | Estrogen-related receptor alpha |
| ERα | Estrogen receptor alpha |
| ESR1 | estrogen receptor 1 |
| Fak | Focal adhesion kinase |
| FARS2 | alpha subunit of the cytosolic phenylalanyl-tRNA synthetase (Pf PheRS) |
| FER | FER Tyrosine Kinase |
| FES | FES Tyrosine Kinase |
| FKBP12 | Peptidyl-prolyl cis-trans isomerase FKBP12 |
| FLI1 | Fli-1 proto-oncogene, ETS transcription factor |
| FLT3 | Receptor-type tyrosine-protein kinase FLT3 |
| FOXA1 | forkhead box A1 |
| GAK | Cyclin G-associated kinase |
| GATA3 | GATA binding protein 3 |
| GATA6 | GATA binding protein 6 |
| GSK3 | Serine/threonine protein kinase 3 |
| HAVCR2 | hepatitis A virus cellular receptor 2 |
| HBV | Hepatitis B virus - capsid |
| HCMV | cytomegalovirus - capsid |
| HCV | Hepatitis C virus- - capsid |
| HDAC1 | Histone Deacetylase 1 |
| HDAC10 | Histone Deacetylase 10 |
| HDAC11 | Histone Deacetylase 11 |
| HDAC2 | Histone Deacetylase 2 |
| HDAC3 | Histone Deacetylase 3 |
| HDAC4 | Histone Deacetylase 4 |
| HDAC5 | Histone Deacetylase 5 |
| HDAC6 | Histone Deacetylase 6 |
| HDAC7 | Histone Deacetylase 7 |
| HDAC8 | Histone Deacetylase 8 |
| HDAC9 | Histone Deacetylase 9 |
| HER2 | Human epidermal growth factor receptor 2 |
| HHV 6A and 6B | Human herpesvirus 6A and 6B - capsid |
| HHV 7 | Human herpesvirus 7 - capsid |
| HHV-8 | Kaposi's sarcoma-associated herpesvirus - capsid |
| HIF1A | hypoxia inducible factor 1 subunit alpha |
| HIV | Human immunodeficiency virus -- capsid |
| HNF1A | HNF1 homeobox A |
| HNF1B | HNF1 homeobox B |
| HNF4A | hepatocyte nuclear factor 4 alpha |
| HRV | Rhinovirus and enteroviruses, including polioviruses, Coxsackie A |
| | viruses (CA), Coxsackie B viruses (CB), and echoviruses |
| HSD17b13 | hydroxysteroid 17-beta dehydrogenase 13 |
| HSV1 | Herpes simplex viruses 1 - capsid |
| HSV1 | Herpes simplex viruses 2 - capsid |
| HTT | Huntingtin |
| ICOS | Inducible T cell costimulator |
| IDH1 | Isocitrate dehydrogenase (NADP(+)) 1, including R132H/S/G/L/I |
| IDH2 | Isocitrate dehydrogenase (NADP(+)) 2, including R132H/S/G/L/I |
| IKZF1 | Ikaros transcription factors |
| IKZF3 | Aiolos transcription factors |
| IL15 | interleukin 15 |
| IL27 | interleukin 27 |
| IRAK1 | Interleukin-1 receptor-associated kinase 1 |
| IRAK1/4 | Interleukin-1 receptor-associated kinase 4 |
| IRF4 | interferon regulatory factor 4 |
| ITK | Interleukin-2-inducible T-cell kinase |
| JAK1 | Janus kinase 1 |
| JAK2 | Janus kinase 2 |
| JAK2 | Janus kinase 2, WT abd variant V617F |
| KARS1 | Lysyl-tRNA-Synthetase |
| KLF5 | Kruppel like factor 5 |
| KLRK1 | killer cell lectin like receptor K1 |
| KRAS | K-Ras P21 Protein |
| KRAS | KRAS proto-oncogene, GTPase, WT and variants G12V, C, D or G13D |
| KRAS G12V/C/D | mutant K-Ras P21 Protein |
| KRAS G13D | mutant K-Ras P21 Protein |
| LAG3 | lymphocyte activating 3 |
| LATS1 | Large Tumor Suppressor Kinase 1 |
| LATS2 | Large Tumor Suppressor Kinase 2 |
| LCAT | Lecithin-Cholesterol Acyltransferase |
| LCK | Protein tyrosine kinase Lck |
| LIMK2 | Leucine-rich repeat kinase 2 |
| LMO2 | LIM domain only 2 |
| M1 and M17 | M1 and M17 aminopeptidases |
| aminopeptidases | |
| MAP2K1 | mitogen-activated protein kinase kinase 1 |
| MAP2K2 | mitogen-activated protein kinase kinase 2 |
| MAPK1 | mitogen-activated protein kinase 1 |
| MAPK3 | mitogen-activated protein kinase 3 |
| MARK2 | Microtubule Affinity Regulating Kinase 2 |
| MAX | MYC associated factor X |
| MCL1 | MCL1 apoptosis regulator, BCL2 family member |
| MetAP-2 | Methionyl Aminopeptidase 2 |
| MICA | MHC class I polypeptide-related sequence A |
| MICB | MHC class I polypeptide-related sequence B |
| MITF | melanocyte inducing transcription factor |
| MLKL | Mixed lineage kinase domain-like protein |
| MLL | KMT2A (MLL) lysine methyltransferase 2A |
| MPL | MPL proto-oncogene, thrombopoietin receptor, WT and variant |
| | W515S, L or A |
| MRK | MO15-related protein kinase |
| MUC1 | mucin 1, cell surface associated |
| MYB | MYB proto-oncogene, transcription factor |
| MYBL2 | MYB proto-oncogene like 2 |
| MYC | MYC proto-oncogene, bHLH transcription factor |
| MYCN | MYCN proto-oncogene, bHLH transcription factor |
| MyD88 | Myeloid Differentiation Primary Response 88 |
| MYOC | Myocilin |
| NFE2L2 | nuclear factor, erythroid 2 like 2 |
| NPM1 | nucleophosmin 1, WT and variant NPM1c (AML frameshift mut) |
| Nr2E3 | Photoreceptor-Specific Nuclear Receptor |
| NRAS | NRAS proto-oncogene, GTPase, WT and variants Q61R, K, or L |
| Nrl | Neural Retina Leucine Zipper |
| NT5E | 5′-nucleotidase ecto |
| NTRK | SLIT and NTRK 3 [neurotrophic receptor tyrosine kinase 3] |
| p16INK4a | CDKN2A (P16INK4A) cyclin dependent kinase inhibitor 2A |
| p21CIP/WAF1 | Zinc finger protein Gfi-1? |
| p38 | Mitogen-activated protein (MAP) kinase (MAPK11) |
| PARS2 | cytoplasmic prolyl-tRNA (transfer RNA) synthetase (PfcPRS) |
| PAX8 | paired box 8 |
| PDCD1 | programmed cell death 1 |
| PDE4 | Phosphodiesterase 4 |
| PI3K34 | Phosphatidylinositol 3-kinase 34 |
| PIK3CA | phosphatidylinositol-4,5-bisphosphate 3-kinase catalytic subunit alpha |
| | WT and variants H1047R or E545K |
| PIK3CB | Phosphatidylinositol-4,5-bisphosphate 3-kinase catalytic subunit beta |
| PIK3CD | Phosphatidylinositol-4,5-bisphosphate 3-kinase catalytic subunit delta |
| PKA | cAMP dependent protein kinase |
| PKG | Protein kinase G |
| Plasmepsin IX | Plasmepsin IX |
| Plasmepsin X | Plasmepsin X |
| PNPLA2 | Patatin like phospholipase domain containing 2 |
| PNPLA3 | Patatin like phospholipase domain containing 2 |
| POU2AF1 | POU class 2 homeobox associating factor 1 |
| PPP1R12B | Protein phosphatase 1 regulatory subunit 12B |
| PRKAA1 | Protein Kinase AMP-Activated Catalytic Subunit Alpha 1 |
| PRMT5 | Protein arginine methyltransferase 5 |
| PRS | Prolyl-tRNA-Synthetase |
| PSD-95 | Post synaptic density protein 95, a membrane associated guanylate |
| | kinase |
| PTK2 | Protein Tyrosine Kinase 2 |
| PTK2B | Protein Tyrosine Kinase 2 beta |
| PTPN11 | protein tyrosine phosphatase non-receptor type 11 |
| PTPN2 | protein tyrosine phosphatase non-receptor type 2 |
| PTPN6 | protein tyrosine phosphatase non-receptor type 6 |
| PTPRB | Protein Tyrosine Phosphatase Receptor Type B |
| RAC1 | Rac family small GTPase 1, WT and variant P29S |
| RAET1E | retinoic acid early transcript 1E |
| RAET1G | retinoic acid early transcript 1G |
| RAET1L | retinoic acid early transcript 1L |
| RAF1 | Raf-1 proto-oncogene, serine/threonine kinase |
| RAR | Retinoic acid receptor |
| RET | ret proto-oncogene |
| RHOA | ras homolog family member A |
| RHOB | ras homolog family member B |
| RIPK2 | Receptor Interacting Serine/Threonine Kinase 2 |
| ROCK 1 | Rho Associated Coiled-Coil Containing Protein Kinase 1 |
| RSV | Human respiratory syncytial virus - capsid |
| RUNX1 | RUNX family transcription factor 1 |
| SARM1 | Sterile Alpha And TIR Motif-Containing Protein 1 |
| SHOC2 | SHOC2 leucine rich repeat scaffold protein |
| Sirt 1 | Sirtuin 1 |
| Sirt 2 | Sirtuin 2, an NAD-dependent deacetylase |
| Sirt 3 | Sirtuin 3 |
| Smad3 | SMAD Family Member 3 |
| SMARCA2 | SWI/SNF related, matrix associated, actin dependent regulator of |
| | chromatin, subfamily a, member 2 |
| SMARCA4 | SWI/SNF related, matrix associated, actin dependent regulator of |
| | chromatin, subfamily a, member 4 |
| SNAIL1 | Snail Family Transcriptional Repressor 1 |
| SNCA | Alpha synuclein |
| SOX10 | SRY-box transcription factor 10 |
| SPI1 | Spi-1 proto-oncogene |
| SPTLC1 | Serine palmitoyltransferase 1 |
| STAT1 | Signal transducer and activator of transcription 1 |
| STAT2 | Signal transducer and activator of transcription 2 |
| STAT3 | Signal transducer and activator of transcription 3 |
| STAT4 | Signal transducer and activator of transcription 4 |
| STAT5 | Signal transducer and activator of transcription 5 |
| STAT6 | Signal transducer and activator of transcription 6 |
| STK17A | Death-Associated Protein Kinase-Related 1 |
| STK17B | Death-Associated Protein Kinase-Related 2 |
| TACC3 | Transforming Acidic Coiled-Coil Containing Protein 3 |
| TANK | TRAF Family Member Associated NFKB Activator |
| Tau | Tau protein |
| TBK1 | TANK binding kinase 1 |
| TCF7L2 | transcription factor 7 like 2 |
| TDP43 | TAR DNA-binding protein 43 |
| TEAD1 | TEA domain transcription factor 1 |
| TEAD2 | TEA domain transcription factor 2 |
| TEAD3 | TEA domain transcription factor 3 |
| TEAD4 | TEA domain transcription factor 4 |
| TEC | Tec non-receptor protein-tyrosine kinase |
| TFAP2A | Transcription factor AP-2 alpha |
| TFAP2C | Transcription factor AP-2 gamma |
| TGFB1 | Transforming growth factor beta 1 |
| TIGIT | T cell immunoreceptor with Ig and ITIM domains |
| TLR7 | Toll like receptor 7 |
| TMEM173/STING1 | Stimulator of interferon response cGAMP interactor 1 |
| TNFRSF18 | TNF receptor superfamily member 18 |
| TNNI3K | TNNI3 Interacting Kinase |
| TP63 | tumor protein p63 |
| TRIM24 | Tripartite Motif Containing 24; transcriptional intermediary factor 1α |
| TYK2 | Tyrosine Kinase 2 |
| ULBP1 | UL16 binding protein 1 |
| ULBP2 | UL16 binding protein 2 |
| ULBP3 | UL16 binding protein 3 |
| ULK1 | Unc-51 Like Autophagy Activating Kinase 1 |
| VHL | Von Hippel-Lindau Tumor Suppressor |
| VZV | Varicella-zoster virus - capsid |
| WEE1 | WEE1 G2 Checkpoint Kinase |
| WRN | WRN RecQ like helicase |
| WWTR1 | WW domain containing transcription regulator 1 |
| XIAP | X-linked inhibitor of apoptosis |
| X-Protein | Hepatitis B X-protein. |
| ZFP64 | ZFP64 zinc finger protein. |

In an embodiment, the Target Protein is a fusion target protein. In an embodiment, the fusion target protein is selected from Table 2:

TABLE 2
Exemplary Fusion Target Proteins

| NCBI | NCBI | Gene Name | Example fusion partners |
|---|---|---|---|
| symbol | gene ID | (extended from NCBI) | (NCBI symbol) |
| RUNX1 | 861 | RUNX family transcription factor 1 | ETV6, MECOM, RUNX1T1 |
| ABL1 | 25 | ABL proto-oncogene 1 | BCR, NUP214, EML1, ETV6 |
| TCF3 | 6929 | transcription factor 3 | PBX1, TFPT, ZNF384, HLF |
| ZNF384 | 171017 | zinc finger protein 384 | EWSR1, TAF15, TCF3, EP300, |
| | | | CREBBP |
| CRLF2 | 64109 | cytokine receptor like factor 2 | P2RY8 |
| MEF2D | 4209 | myocyte enhancer factor 2D | BCL9, SS18, FOXJ2, CSF1R, DAZAP1 |
| PAX5 | 5079 | paired box 5 | ELN, ETV6, AUTS2, POM121, JAK2, |
| | | | FOXP1, NCOR1 |
| TRBC1 | 28639 | T cell receptor beta constant 1 | TAL1, TAL2, LYL1, OLIG2, LMO1, |
| | | | LMO2, TLX1, TLX3, MYC, and MYB |
| TRAC | 28755 | T cell receptor alpha constant | TAL1, TAL2, LYL1, OLIG2, LMO1, |
| | | | LMO2, TLX1, TLX3, MYC, and MYB |
| KMT2A | 4297 | lysine methyltransferase 2A | AFF1, MLLT3, MLLT1, MLLT10, |
| | | | MLLT4, ELL, AFDN, MLLT6, AFF4 |
| FLI1 | 2313 | Fli-1 proto-oncogene | EWSR1 |
| ERG | 2078 | ETS transcription factor ERG | TMPRSS2, EWSR1, SLC45A3, FUS, |
| | | | ETV6 |
| ETV1 | 2115 | ETS variant transcription factor 1 | TMPRSS2, EWSR1, SLC45A3, ACSL3, |
| | | | FOXP1 |
| ETV4 | 2118 | ETS variant transcription factor 4 | TMPRSS2, EWSR1, KLK2, CANT1, |
| | | | DDX5 |
| ETV5 | 2119 | ETS variant transcription factor 5 | TMPRSS2, SLC45A3 |
| PML | 5371 | promyelocytic leukemia | RARA |
| NUP98 | 4928 | nucleoporin 98 | NSD1, HOXA9, PRRX1, HOXA11, |
| | | | HOXA13, HOXC11, HOXC13 |
| CBFA2T3 | 863 | CBFA2/RUNX1 partner | GLIS2 |
| | | transcriptional co-repressor 3 | |
| CBFB | 865 | core-binding factor, beta subunit | MYH11 |
| KAT6A | 7994 | lysine acetyltransferase 6A | CREBBP, EP300, NCOA2 |
| FGFR3 | 2261 | fibroblast growth factor receptor 3 | TACC3, BAIAP2L1 |
| FGFR2 | 2263 | fibroblast growth factor receptor 2 | BICC1, KLK2, CIT, CCAR2 |
| FGFR1 | 2260 | fibroblast growth factor receptor 1 | FN1, TACC1 |
| RET | 5979 | ret proto-oncogene | CCDC6, ERC1, NCOA4, KIF5B, |
| | | | TRIM33, CUX1, CLIP1, RUFY3 |
| ALK | 238 | anaplastic lymphoma receptor | EML4, KIF5B, RANBP2, TFG, |
| | | tyrosine kinase | PRKAR1A, TPM3, NPM1 |
| NTRK3 | 4916 | neurotrophic tyrosine kinase, | ETV6, BTBD1, AML4, TFG, LYN, |
| | | receptor, type 3 | RBPMS |
| NTRK2 | 4915 | neurotrophic receptor tyrosine | QKI, NACC2, VCL, AGBL4, PAN3, |
| | | kinase 2 | AFAP1, DAB2IP |
| NTRK1 | 4914 | neurotrophic receptor tyrosine | CD74, MPRIP, TPM3, TFG, TPR |
| | | kinase 1 | |
| ROS1 | 6098 | ROS proto-oncogene 1, receptor | CD74, EZR, TPM3, SDC4, LRIG3, FIG, |
| | | tyrosine kinase | CCDC6, GOPC |
| RSPO3 | 84870 | R-spondin 3 | PTPRK |
| RSPO2 | 340419 | R-spondin 2 | EIFE3 |
| SMYD3 | 64754 | SET and MYND domain containing | KIF26B |
| | | 3 | |
| MAML3 | 55534 | mastermind like transcriptional | UBTF, PAX3, BCOR, TCF4 |
| | | coactivator 3 | |
| MAML2 | 84441 | mastermind like transcriptional | CRTC1 |
| | | coactivator 2 | |
| TFE3 | 7030 | transcription factor binding to IGHM | SFPQ, PRCC, ASPSCR1, NONO, |
| | | enhancer 3 | CLTC |
| TFEB | 7942 | transcription factor EB | MALAT1 |
| EGFR | 1956 | epidermal growth factor receptor | SEPT14 |
| BRAF | 673 | B-Raf proto-oncogene, | KIAA1549, PAPSS1, TRIM24, |
| | | serine/threonine kinase | TAX1BP1, CDC27, SND1 |
| SS18 | 6760 | SS18 subunit of BAF chromatin | SSX1, SSX2, SSX4 |
| | | remodeling complex | |
| PAX3 | 5077 | paired box 3 | FOXO1, MLLT7, NCOA1 |
| PAX7 | 5081 | paired box 7 | FOXO1 |
| EWSR1 | 2130 | EWS RNA binding protein 1 | DDIT3, CREB3L1, CREB3L2, CREB1, |
| | | | PBX1, ATF1, NR4A3 |
| FUS | 2521 | FUS RNA binding protein | ERG, CREB3L2, DDIT3 |
| CIC | 23152 | capicua transcriptional repressor | DUX4, DUX4L10, FOXO4 |
| FOSB | 2354 | FosB proto-oncogene, AP-1 | ZFP36, SERPINE1 |
| | | transcription factor | |
| VGLL2 | 245806 | vestigial like family member 2 | CITED2, NCOA2 |
| VAV1 | 7409 | vav guanine nucleotide exchange | GSS, MYO1F |
| | | factor 1 | |
| STAT6 | 6778 | signal transducer and activator of | NAB2 |
| | | transcription 6 | |
| HMGA2 | 8091 | high mobility group AT-hook 2 | LPP |
| PDGFRB | 5159 | platelet derived growth factor | ETV6 |
| | | receptor beta | |
| BRD4 | 23476 | bromodomain containing 4 | NUTM1 |
| PAX8 | 7849 | paired box 8 | PPARG |

Another embodiment is a method of treating a target protein-mediated disorder, disease, or condition in a patient comprising administering to the patient any of the compounds described herein, or a pharmaceutically acceptable salt, hydrate, solvate, prodrug, stereoisomer, or tautomer thereof. In an embodiment, the disorder is selected from a respiratory disorder, a proliferative disorder, an autoimmune disorder, an autoinflammatory disorder, an inflammatory disorder, a neurological disorder, and an infectious disease or disorder. In an embodiment, the disorder is a proliferative disorder. In an embodiment, the proliferative disorder is cancer.

Another embodiment is a method of treating or preventing a disease mediated by BTK in a subject in need thereof, the method comprising administering to the subject a therapeutically effective amount of any of the compounds described herein, or a pharmaceutically acceptable salt, hydrate, solvate, prodrug, stereoisomer, or tautomer thereof.

Another embodiment is a method of treating cancer in a subject in need thereof, the method comprising administering to the subject a therapeutically effective amount of any of the compounds described herein, or a pharmaceutically acceptable salt, hydrate, solvate, prodrug, stereoisomer, or tautomer thereof.

Another embodiment is a pharmaceutical composition comprising any of the compounds described herein, or a pharmaceutically acceptable salt, hydrate, solvate, prodrug, stereoisomer, or tautomer thereof, and a pharmaceutically acceptable carrier.

Another embodiment is a pharmaceutical combination comprising any of the compounds described herein, or a pharmaceutically acceptable salt, hydrate, solvate, prodrug, stereoisomer, or tautomer thereof, and a therapeutic agent.

Another embodiment is a method of treating a respiratory disorder, a proliferative disorder, an autoimmune disorder, an autoinflammatory disorder, an inflammatory disorder, a neurological disorder, and an infectious disease or disorder in a subject in need thereof, the method comprising administering to the subject a therapeutically effective amount of any of the compounds described herein, or a pharmaceutically acceptable salt, hydrate, solvate, prodrug, stereoisomer, or tautomer thereof. In an embodiment, the disorder is a proliferative disorder. In an embodiment, the proliferative disorder is cancer.

Another embodiment is the use of any of the compounds described herein, or a pharmaceutically acceptable salt, hydrate, solvate, prodrug, stereoisomer, or tautomer thereof in the preparation of a medicament for treating a respiratory disorder, a proliferative disorder, an autoimmune disorder, an autoinflammatory disorder, an inflammatory disorder, a neurological disorder, and an infectious disease or disorder in a subject in need thereof. One aspect is the use of any of the compounds described herein, or a pharmaceutically acceptable salt, hydrate, solvate, prodrug, stereoisomer, or tautomer thereof for treating cancer.

Another aspect is the use of any of the compounds described herein, or a pharmaceutically acceptable salt, hydrate, solvate, prodrug, stereoisomer, or tautomer thereof in the preparation of a medicament for treating a disease mediated by BTK.

### DETAILED DESCRIPTION

Described herein are compounds or a pharmaceutically acceptable salt, hydrate, solvate, prodrug, stereoisomer, or tautomer thereof that function to recruit targeted proteins to E3 ubiquitin ligase for degradation, methods of preparation thereof, and uses thereof.

In one aspect, the disclosure provides conjugates or a pharmaceutically acceptable salt, hydrate, solvate, prodrug, stereoisomer, or tautomer thereof, which recruit a targeted protein, such as a bromodomain-containing protein or a protein kinase, to E3 ubiquitin ligase for degradation. The conjugates and pharmaceutically acceptable salts, hydrates, solvates, prodrugs, stereoisomers, or tautomers thereof comprise a bifunctional protein degrader as defined herein in a modified form having prolonged exposure thereby resulting in prolonged biological activity. It has been discovered that conjugating these bifunctional protein degraders to a lipophilic acid via a linker results in an improved (i.e. prolonged) exposure, with biological activity prolonged and retained.

The conjugates and pharmaceutically acceptable salts, hydrates, solvates, prodrugs, stereoisomers, or tautomers thereof comprise a bifunctional protein degrader, optionally a solubilizing domain, optionally a cleavable linker, and a fatty acid component to, inter alia, improve the pharmacokinetic and/or pharmacodynamic and/or efficacy of the bifunctional protein degrader. In some embodiments, the conjugates have been observed to advantageously result in decreased clearance and higher exposure of the degrader, e.g., as compared to a bifunctional protein degrader that is not conjugated to a fatty acid, in the tumor, whilst having similar or lower exposure in other organs such as the blood, liver, spleen, kidney and heart.

Without wishing to be bound by theory, the prolonged exposure is thought to result from a slow release of the bound lipophilic acid of the conjugate from albumin. The lipophilic acid of the conjugate binds to albumin, which prolongs exposure of the conjugate by avoiding clearance of the small molecule degrader. The small molecule degrader, which is conjugated through a cleavable linker, is then released from the long acting conjugate. This slow release of the small molecule degrader leads to prolonged exposure compared to dosing of the unconjugated small molecule degrader.

In another embodiment, the linker is absent and the Bifunctional Protein Degrader is covalently linked, i.e., directly linked through a covalent bond, to the Fatty Acid.

In one aspect, the disclosure provides a conjugate of Formula (I):

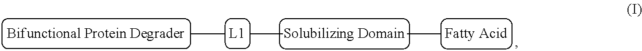

or a pharmaceutically acceptable salt, hydrate, solvate, prodrug, stereoisomer, or tautomer thereof, wherein
(i) a bifunctional protein degrader comprises a bifunctional compound capable of binding to each of a target protein and a ligase independently;
(ii) L1 comprises a cleavable linker;
(iii) optionally, a solubilizing domain comprises a heteroalkylene and is soluble in aqueous solution; and
(iv) a fatty acid comprises a fatty acid capable of binding to a protein.

In another aspect, the disclosure provides conjugates and compositions having prolonged exposure and that are capable of modulating or inhibiting a Bruton's tyrosine kinase (BTK) by binding to and altering the specificity of a cereblon complex to induce ubiquitination and degradation of a complex-associated BTK.

In one aspect, the disclosure provides a conjugate of Formula (I′):

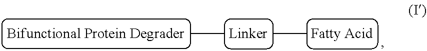

or a pharmaceutically acceptable salt, hydrate, solvate, prodrug, stereoisomer, or tautomer thereof, wherein
(i) a Bifunctional Protein Degrader is a Bruton's tyrosine kinase (BTK) Degrader capable of degrading BTK; and
(ii) a Linker is absent or L⁴, wherein L⁴ is a group that is cleavable to allow release of the Bifunctional Protein Degrader, and that covalently links the Bifunctional Protein Degrader to a Fatty Acid.

In an embodiment, L⁴ comprises L1 and, optionally, a solubilizing domain.

### Target Proteins

In one aspect, the disclosure provides compounds or a pharmaceutically acceptable salt, hydrate, solvate, prodrug, stereoisomer, or tautomer thereof, which recruit a targeted protein, such as a bromodomain-containing protein or a protein kinase, to E3 ubiquitin ligase for degradation. In an embodiment, the target protein is selected from Table 1 or Table 2.

### Bifunctional Protein Degraders

The present disclosure features conjugates comprising a bifunctional protein degrader, having the structure of Formula (I-a):

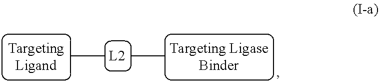

or a pharmaceutically acceptable salt, hydrate, solvate, stereoisomer, or tautomer thereof, wherein (i) a targeting ligand comprises an entity capable of binding to the target protein; (ii) L2 is a linker; and (iii) a targeting ligase binder comprises an entity capable of binding the ligase.

### Targeting Ligands

The Targeting Ligand is a small molecule moiety that is capable of binding to a target protein or protein of interest (POI). In an embodiment, the target protein or POI is a target protein selected from Table 1. In an embodiment, the target protein or POI is a fusion protein. In an embodiment, the target protein or POI is a target protein selected from Table 2.

In an embodiment, the targeting ligand is a BTK targeting ligand. In an embodiment, the Targeting Ligand is a BTK targeting ligand of Formula (BTK-I):

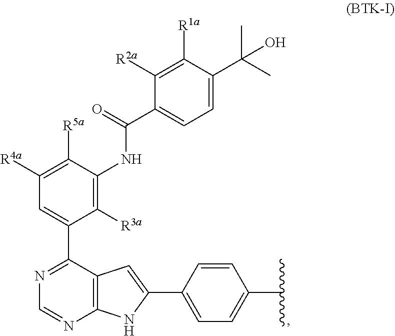

or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo; and
R^{5a} is H or halo.

Additional exemplary Targeting Ligands include, but are not limited to, the targeting ligands in Table 3:

TABLE 3
Exemplary Targeting Ligands

| Gene | Targeting Ligand |
|---|---|
| ALK | ceritinib |
| AR | 7a-methyl-19-nortestosterone |
| AR | Testosterone |
| BCR-ABL | dasatinib |
| BRD4 | JQ1 |
| BRD9 | BI-7273 |
| BTK | bosutinib |
| BTK | RN486 |
| CDK9 | SNS-032 |
| c-Met | foretinib |
| EGFR | AC480 |
| EGFR | ARRY-334543 |
| EGFR | afatinib |
| ERα | 4-OHT |
| EGFR Δ Exon 19 | gefitinib |
| FKBP12 | Steel Factor |
| FLT3 | 4SC-202 |
| FLT3 | Linifanib |
| FLT3 | Quizartinib (AC220) |
| JAK2 | AC430 |
| HDAC4 | AR-42 |
| HDAC6 | ACY-1215 |
| HDAC6 | AR-42 |
| TRIM24 | IACS-7e |

wherein the Targeting Ligand is attached to the Linker-Targeting Ligase Binder, e.g.,

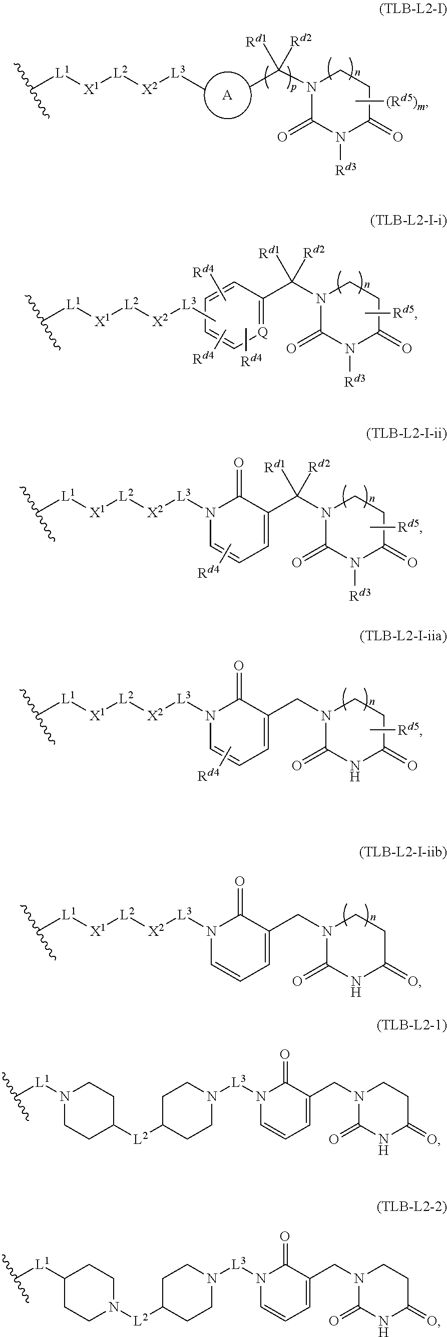

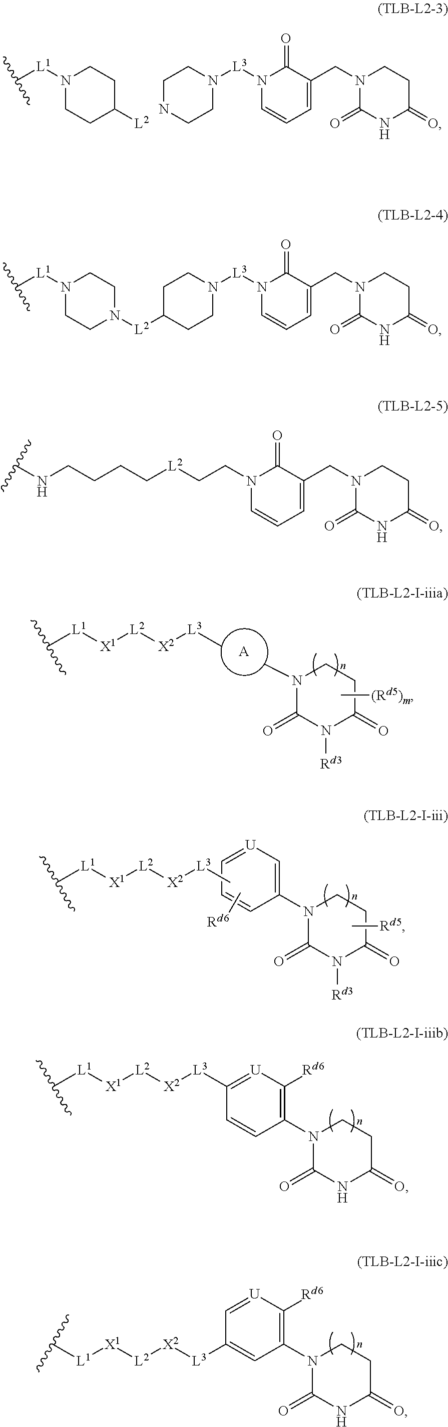

through a modifiable carbon, oxygen, nitrogen or sulfur atom on the Targeting Ligand.

In an embodiment, the Targeting Ligand is a targeting ligand described in Huang et al., “A Chemoproteomic Approach to Query the Degradable Kinome Using a Multi-kinase Degrader,” Cell Chem. Biol. 25(1): 88-99 (2018); An and Fu, “Small-molecule PROTACs: An emerging and promising approach for the development of targeted therapy drugs,” EBioMedicine 36: 553-562 (2018); Pei et al., “Small molecule PROTACs: an emerging technology for targeted therapy in drug discovery,” RSC Adv. 9:16967-16976 (2019); and Zou et al., Cell Biochem. Funct. 37: 21-30 (2019), each of which is incorporated by reference herein in its entirety.

In an embodiment, the Targeting Ligand is selected from the group consisting of:

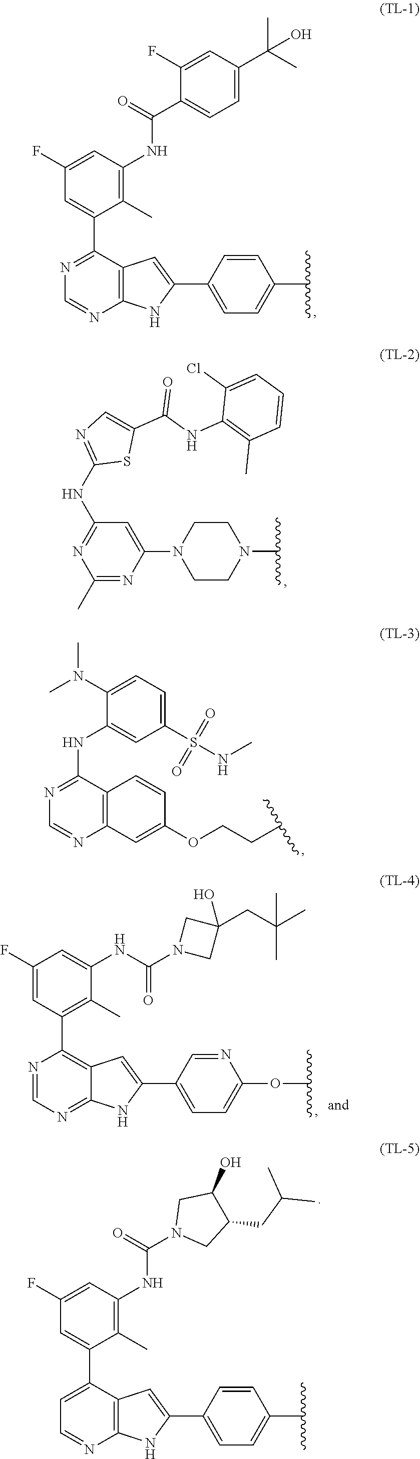

### Targeting Ligase Binder

The Targeting Ligase Binder brings a protein of interest (POI) into close proximity to a ubiquitin ligase for tagging with Ubiquitin (Ub), marking the POI for degradation by the ligase through the linking of the Targeting Ligase Binder bound to the ubiquitin ligase (e.g., an E3 Ubiquitin ligase binding complex), Linker (L), and a Targeting Ligand (TL) bound to the POI. See e.g., FIG. 1.

In an embodiment, the Targeting Ligase Binder has a Formula (TLB-I):

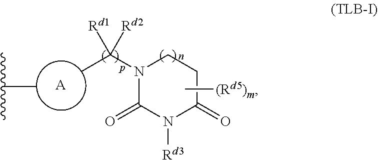

or a pharmaceutically acceptable salt, hydrate, solvate, prodrug, stereoisomer, or tautomer thereof, wherein:

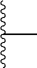

denotes the point of attachment to L2 in Formula (I-a);
Ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{p} is H or C₁₋₆ alkyl;
m is 1 or 2;
n is 1 or 2; and
p is 0, 1, or 2.

In an embodiment, n is 1. In an embodiment, p is 1. In an embodiment, R^{d3} is H. In an embodiment, R^{d3} is —CH₂OP(O)(OR^{p})₂.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered nitrogen-containing heteroaryl or a 6-membered nitrogen-containing heteroaryl (e.g., a nitrogen-containing heteroaryl). In an embodiment, ring A is a 5-membered heteroaryl. In an embodiment, A is a 5-membered nitrogen-containing heteroaryl. In an embodiment, A is a 6-membered heteroaryl. In an embodiment, ring A is a 6-membered nitrogen-containing heteroaryl. In an embodiment, ring A is pyridyl or pyridonyl. In an embodiment, R^{d4} is hydroxyl or C₁₋₆ alkoxyl.

In an embodiment, the Targeting Ligase Binder has a Formula (TLB-I-i):

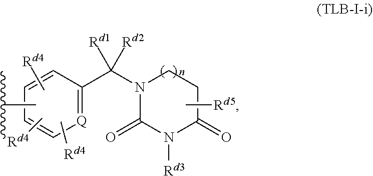

or a pharmaceutically acceptable salt, hydrate, solvate, prodrug, stereoisomer, or tautomer thereof, wherein:

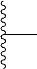

denotes the point of attachment to L2 in Formula (I-a);
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂.

In an embodiment, R^{d4} is hydroxyl or C₁₋₆ alkoxyl.

In another embodiment, the Targeting Ligase Binder has a Formula (TLB-I-ii):

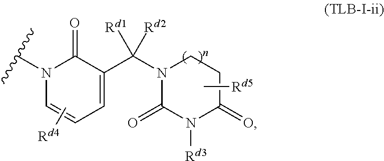

or a pharmaceutically acceptable salt, hydrate, solvate, prodrug, stereoisomer, or tautomer thereof,
wherein:

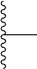

denotes the point of attachment to the L2 in Formula (I-a);
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂. In an embodiment, R^{d1} is H. In an embodiment, R^{d2} is H. In an embodiment, R^{d1} and R^{d2} are both H.

In an embodiment, the Targeting Ligase Binder has a Formula (TLB-I-iia):

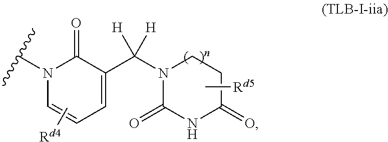

or a pharmaceutically acceptable salt, hydrate, solvate, prodrug, stereoisomer, or tautomer thereof, wherein:

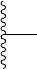

denotes the point of attachment to L2 in Formula (I-a);
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl; and
n is 1 or 2.

In an embodiment, n is 1. In an embodiment, R^{d3} is H. In an embodiment, R^{d3} is —CH₂OP(O)(OR^{p})₂. In an embodiment, R^{d4} is H or C₁₋₃ alkyl. In an embodiment, R^{d4} is H. In an embodiment, R^{d5} is H or C₁₋₃ alkyl. In an embodiment, R^{d5} is H.

In another embodiment, the Targeting Ligase Binder has a Formula (TLB-I-iib):

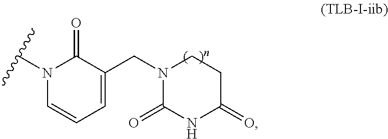

or a pharmaceutically acceptable salt, hydrate, solvate, prodrug, stereoisomer, or tautomer thereof.

In an embodiment, the Targeting Ligase Binder has a Formula (TLB-I-iiia):

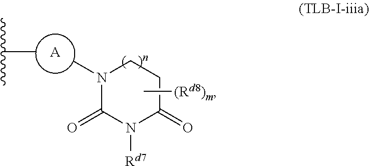

or a pharmaceutically acceptable salt, hydrate, solvate, prodrug, stereoisomer, or tautomer thereof, wherein:

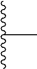

denotes the point of attachment to L2 in Formula (I-a);
Ring A is a 6-membered aryl or 6-membered heteroaryl, each of which is independently substituted with 0-4 occurrences of R^{d6}.
each R^{d6} is independently selected from the group consisting of H, hydroxyl, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
m is 1 or 2; and
n is 1 or 2.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a nitrogen-containing 6-membered heteroaryl. In an embodiment, ring A is pyridyl.

In an embodiment, n is 1. In an embodiment, n is 2. In an embodiment, R^{d7} is —CH₂OP(O)(OR^{p})₂. In an embodiment, R^{d7} is H. In an embodiment, R^{d8} is H. In an embodiment, R^{d7} and R^{d8} are both H. In an embodiment, R^{d6} is H. In an embodiment, R^{d6} is selected from the group consisting of H, halogen, C₁₋₆ alkyl, and C₁₋₆ alkoxyl. In an embodiment, R^{d6} is selected from the group consisting of H, halogen, C₁₋₆ alkyl, and C₁₋₆ alkoxyl; and R^{d7}, and R^{d8} are each H.

In an embodiment, the Targeting Ligase Binder has a Formula (TLB-I-iii):

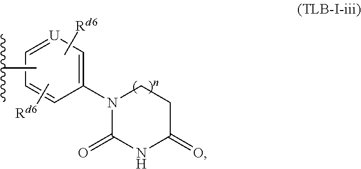

or a pharmaceutically acceptable salt, hydrate, solvate, prodrug, stereoisomer, or tautomer thereof, wherein:

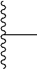

denotes the point of attachment to the L2 in Formula (I-a);
U is —CR^{d6} or N;
each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, n is 1. In an embodiment, n is 2. In an embodiment, each R^{d6} is independently selected from the group consisting of H, halogen, C₁₋₃ alkyl, and C₁₋₃ alkoxy. In an embodiment, each R^{d6} is H. In an embodiment, one of R^{d6} is H. In an embodiment, one of R^{d6} is not H.

In an embodiment, the Targeting Ligase Binder has a Formula (TLB-I-iiib):

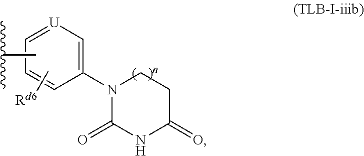

or a pharmaceutically acceptable salt, hydrate, solvate, prodrug, stereoisomer, or tautomer thereof, wherein:

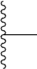

denotes the point of attachment to L2 in Formula (I-a);
U is —CR^{d6} or N;
R^{d6} is selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, the Targeting Ligase Binder has a Formula (TLB-I-iiic):

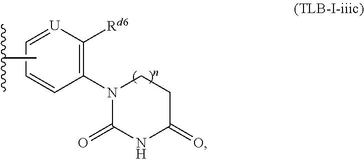

or a pharmaceutically acceptable salt, hydrate, solvate, prodrug, stereoisomer, or tautomer thereof, wherein:

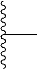

denotes the point of attachment to L2 in Formula (I-a);
U is independently —CR^{d6} or N;
R^{d6} is selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, n is 1. In an embodiment, n is 2. In an embodiment, U is N. In an embodiment, U is —CR^{d6}. In an embodiment, each R^{d6} is independently selected from the group consisting of H, methyl, halogen, methoxy, and methoxymethyl. In an embodiment, R^{d6} is H. In an embodiment, R^{d6} is methyl. In an embodiment, R^{d6} is halogen. In an embodiment, R^{d6} is methoxy.

### Linker of the Bifunctional Protein Degrader

In an embodiment, the Linker L2 is a moiety that covalently links, i.e., attaches or connects, the Targeting Ligand to the Targeting Ligase Binder in Formula (I-a).

In an embodiment, the Linker L2 has Formula (L-I):

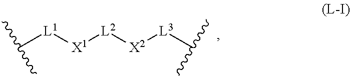

or a pharmaceutically acceptable salt, hydrate, solvate, prodrug, stereoisomer, or tautomer thereof,
wherein:
L¹ is selected from the group consisting of a bond, O, NR′, C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand in Formula (I-a);
X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen;
L² is selected from the group consisting of a bond, O, NR′, C(O), C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl, which is substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen;
L³ is selected from a bond, C₁₋₆ alkylene, C₂₋₆ alkenylene, C₂₋₆ alkynylene, C₁₋₆ heteroalkylene, C(O), S(O)₂, O, NR′, *C(O)—C₁₋₉ alkylene, *C(O)—C₁₋₆ alkylene-O, and *C(O)—C₁₋₉ heteroalkylene, wherein * denotes the point of attachment of L³ to X² in (L-I);
wherein no more than 2 of L¹, X¹, X², L², and L³ can simultaneously be a bond; and
R′ is hydrogen or C₁₋₆ alkyl.

In an embodiment, L¹ is —O—, C₁₋₉ alkylene, e.g., —CH₂— or —CH₂CH₂—, or C₁₋₉ heteroalkylene, e.g., —O—CH₂CH₂—. In an embodiment, L¹ is —O— or C₁₋₉ alkylene. In an embodiment, L¹ is C(O).

In an embodiment, L³ is selected from the group consisting of a bond, —O—, —C(O)—, —S(O)₂—, C₁₋₆ alkylene, C₂₋₆ alkynylene, and C₁₋₆ heteroalkylene.

In an embodiment, one of X¹ and X² is not a bond. In an embodiment, one of X¹ and X² is a bond, and the other is a carbocyclyl or heterocyclyl, wherein the carbocyclyl and heterocyclyl are substituted with 0-4 occurrences of R^{a}, wherein each R^{a} is independently selected from the group consisting of C₁₋₆ alkyl, C₁₋₆ alkoxyl, and halogen. In an embodiment, one of X¹ and X² is a bond, and the other is a heterocyclyl, wherein the heterocyclyl is substituted with 0-4 occurrences of R^{a}, wherein each R^{a} is independently selected from the group consisting of C₁₋₆ alkyl, C₁₋₆ alkoxyl, and halogen.

In an embodiment, X¹ and X² are each independently selected from the group consisting of cyclohexyl, piperidinyl, and piperazinyl, wherein the cyclohexyl, piperidinyl, and piperazinyl are substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

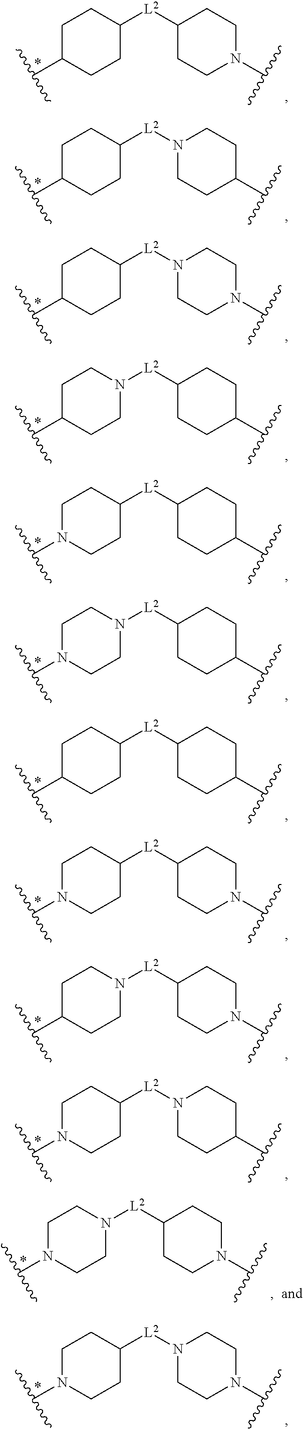

wherein the cyclohexyl, piperidinyl, and piperazinyl are substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹.

In an embodiment, X¹ and X² are each independently selected from piperidinyl and piperazinyl, wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen. In an embodiment, X¹ and X² are both piperidinyl, wherein each piperidinyl is substituted with 0-4 occurrences of R^{a}, wherein each R^{a} is independently selected from the group consisting of C₁₋₆ alkyl, C₁₋₆ alkoxyl, and halogen.

In an embodiment, —X¹-L²-X²— is selected from the group consisting of

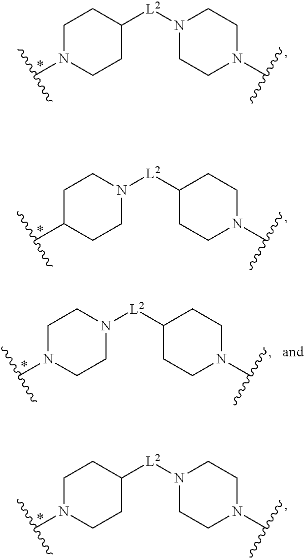

wherein each piperidinyl and piperazinyl is substituted with 0-4 occurrences of R^{a}, wherein each R^{a} is independently selected from the group consisting of C₁₋₆ alkyl, C₁₋₆ alkoxyl, and halogen, or a pharmaceutically acceptable salt, hydrate, solvate, prodrug, stereoisomer, or tautomer thereof, wherein * denotes the point of attachment to L¹. In an embodiment, —X¹-L²-X²— is:

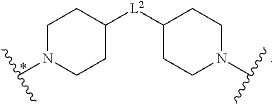

In an embodiment, L² is selected from the group consisting of O, C₁₋₆ alkylene, and C₁₋₆ heteroalkylene. In an embodiment, L² is —CH₂—, O, or C₁₋₃ heteroalkylene. In an embodiment, L² is oxygen. In an embodiment, L₂ is —CH₂—.

In an embodiment, each R^{a} is halogen. In an embodiment, each R^{a} is fluoro.

In an embodiment, —X¹-L²-X²— forms a spiroheterocyclyl having the structure,

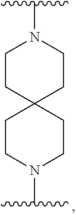 [SMILES: *N1CCC2(CC1)CCN(*)CC2]

substituted with 0-4 occurrences of R^{b}, wherein each R^{b} is independently selected from C₁₋₆ alkyl, C₁₋₆ alkoxyl, C₁₋₆ hydroxyalkyl, and halogen.

In an embodiment, —X¹-L²-X²— forms a spiroheterocyclyl having the structure,

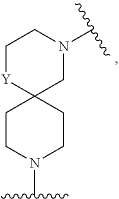

substituted with 0-4 occurrences of R^{b}, wherein Y is selected from CH₂, oxygen, and nitrogen; and each R^{b} is independently selected from C₁₋₆ alkyl, C₁₋₆ alkoxyl, C₁₋₆ hydroxyalkyl, and halogen. In an embodiment, X¹ and X² are each a bond.

In an embodiment, L³ is selected from the group consisting of —O—, —C(O)—, C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O, wherein * denotes the point of attachment of L³ to X². In an embodiment, L³ is selected from the group consisting of —O—, —C(O)—, C₁₋₆ alkylene, C₁₋₆ heteroalkylene, and *C(O)—C₁₋₆ alkylene-O. In an embodiment, L³ is selected from the group consisting of —O—, —C(O)—, C₁₋₃ alkylene, C₁₋₃ heteroalkylene, and *C(O)—C₁₋₃ alkylene-O. In an embodiment, L³ is selected from the group consisting of bond, C₁₋₆ alkylene, C₁₋₆ heteroalkylene, *C(O)—C₁₋₆ alkylene, and *C(O)—C₁₋₆ heteroalkylene. In an embodiment, L³ is selected from the group consisting of C₁₋₆ alkylene, C₁₋₆ heteroalkylene, *C(O)—C₁₋₆ alkylene, and *C(O)—C₁₋₆ heteroalkylene.

In an embodiment, L³ is independently selected from the group consisting of —C(O)—, C₂₋₆ alkynylene, or C₁₋₆ heteroalkylene; and L¹ is —C(O)—, C₁₋₈ alkylene, C₁₋₈ heteroalkylene, and *C₁₋₆ alkylene-C(O). In an embodiment, L³ is selected from the group consisting of —C(O)—, —O—C₁₋₆ alkylene, C₂₋₆ alkynylene, and C₁₋₆ heteroalkylene; and L¹ is C₁₋₈ alkylene or C₁₋₈ heteroalkylene. In an embodiment, L³ is —C(O)— or C₁₋₆ heteroalkylene; and L¹ is C₁₋₈ alkylene or C₁₋₈ heteroalkylene. In an embodiment, L³ is a bond or —O—; and L¹ is —C(O)— or C₁₋₈ heteroalkylene. In an embodiment, L³ is selected from the group consisting of —O—, —C(O)—, —S(O)₂—, and C₁₋₆ heteroalkylene; and L¹ is C₁₋₈ alkylene or C₁₋₈ heteroalkylene. In an embodiment, L² is —C(O)—, —NR′—, or C₁₋₆ alkylene.

In an embodiment, L² is —C(O)—, —O—, or C₁₋₆ alkylene. In an embodiment, L² is C₁₋₆ alkylene. In an embodiment, L² is selected from the group consisting of —C(O)—, C₁₋₆ alkylene, C₁₋₆ heteroalkylene, and *C(O)NR′—C₁₋₆ alkylene.

In an embodiment, Y is CH₂, CH(C₁₋₃ alkyl), C(C₁₋₃ alkyl)₂, oxygen, NH, or N(C₁₋₃ alkyl).

In an embodiment, the Linker L2 is a compound having the following formula:

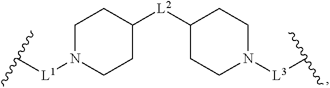

or a pharmaceutically acceptable salt, hydrate, solvate, prodrug, stereoisomer, or tautomer thereof, wherein each piperidinyl is substituted with 0-4 occurrences of R^{a}, wherein each R^{a} is independently selected from the group consisting of C₁₋₆ alkyl, C₁₋₆ alkoxyl, and halogen. In an embodiment, L¹ and L³ are each independently C₁₋₆ alkylene. In an embodiment, L¹ and L³ are each methylene. In an embodiment, L¹ and L³ are each ethylene. In an embodiment, L¹ is methylene and L³ is ethylene. In an embodiment, L² is —CH₂—, O, or C₁₋₃ heteroalkylene. In an embodiment, L² is oxygen. In an embodiment, L² is —CH₂—. In an embodiment, L² is oxygen. In an embodiment, each R^{a} is halogen. In an embodiment, each R^{a} is fluoro.

In an embodiment, the Linker L2 is selected from the group consisting of:

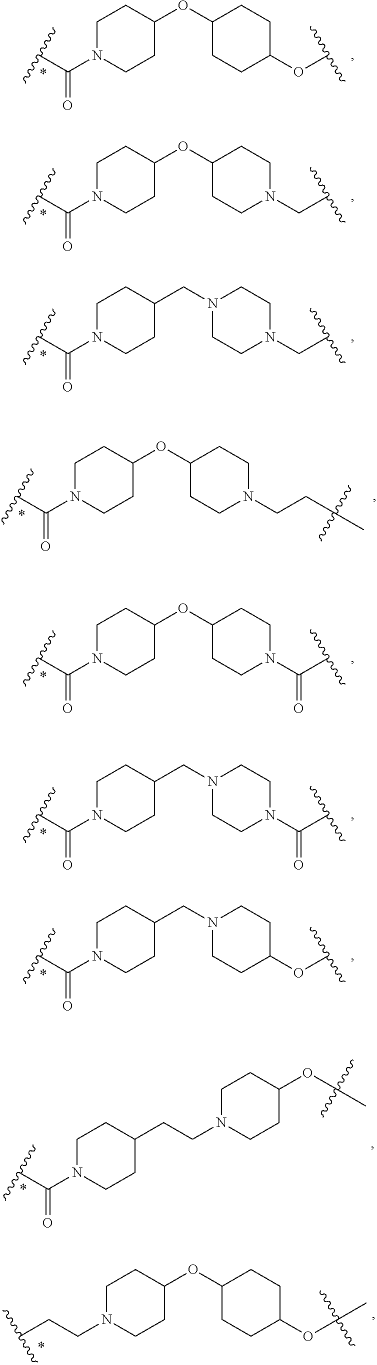

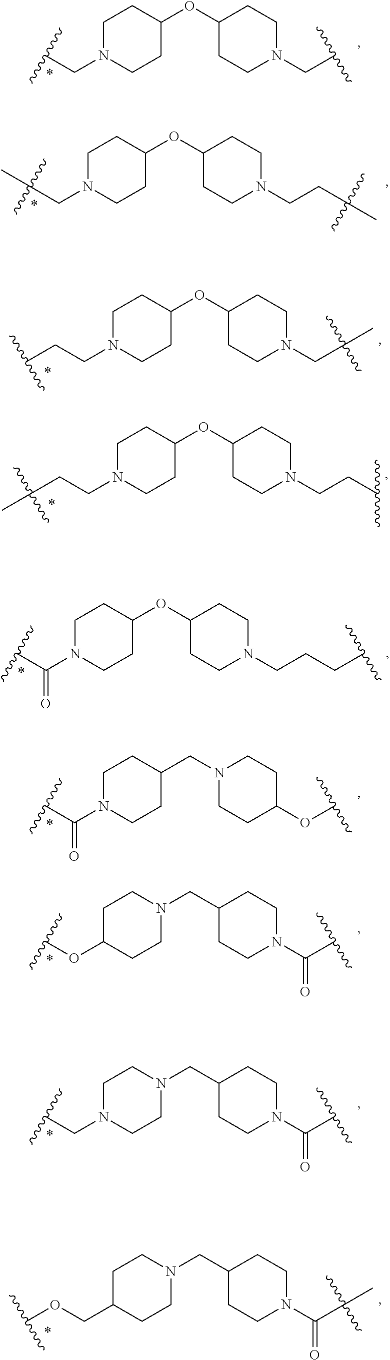

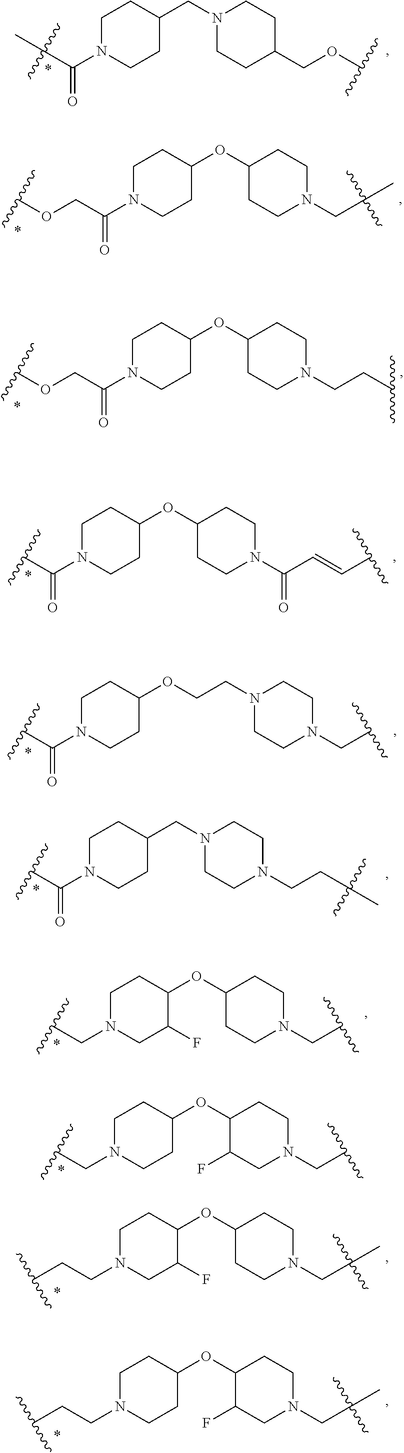

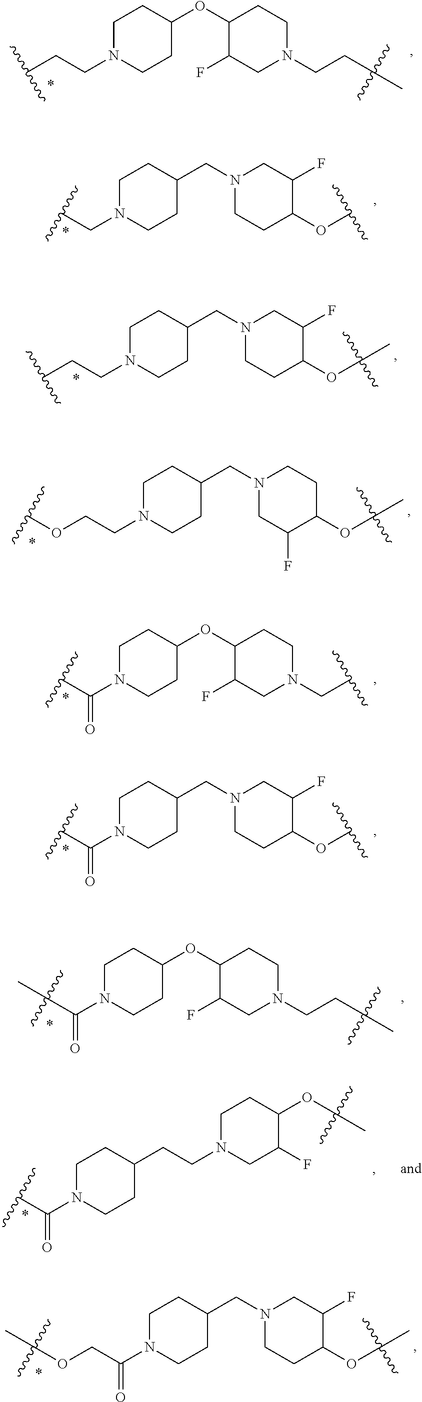

or a pharmaceutically acceptable salt, hydrate, solvate, prodrug, stereoisomer, or tautomer thereof, wherein * denotes the point of attachment to the Targeting Ligase Binder in Formula (I-a).

In an embodiment, the Linker L2 is selected from the group consisting of:

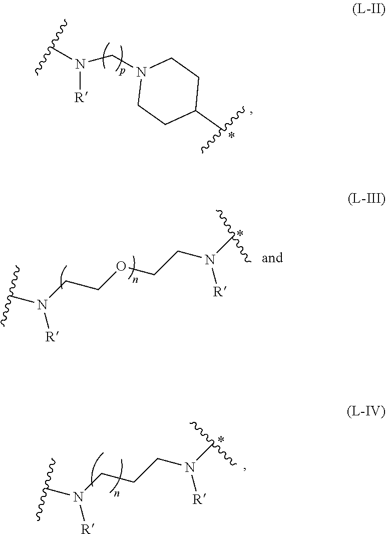

or a pharmaceutically acceptable salt, hydrate, solvate, prodrug, stereoisomer, or tautomer thereof, wherein [symbol] represents the point of attachment to the Targeting Ligand in Formula (I-a); * [symbol] represents the point of attachment to the Targeting Ligase Binder in Formula (I-a); each R′ is independently selected from H and C₁₋₆ alkyl; n is 0 or 1; m is 1, 2, 3, or 4; and p is 2, 3, 4, 5, or 6.

In an embodiment, the Linker L2 is selected from the group consisting of:

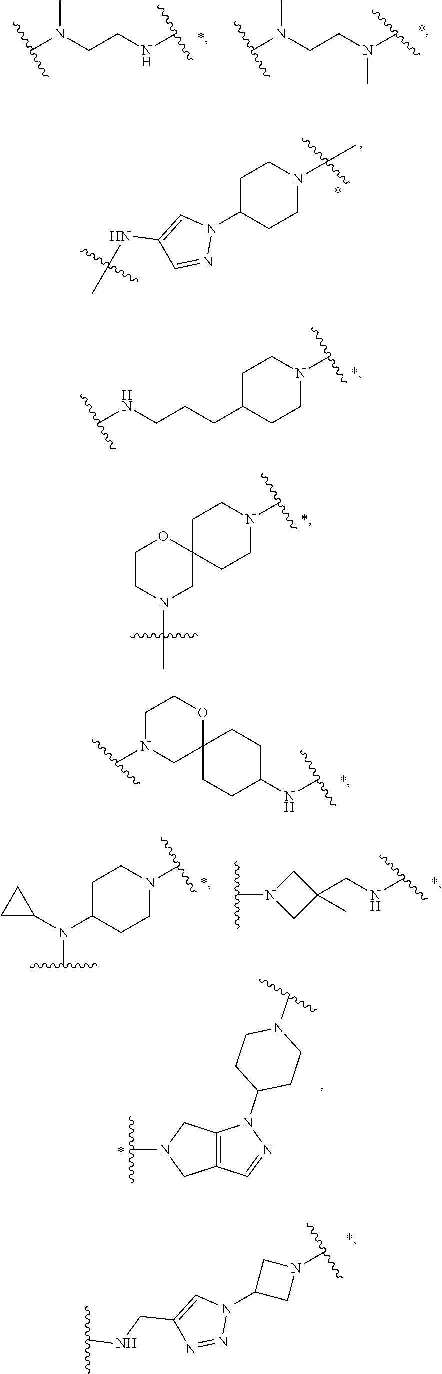

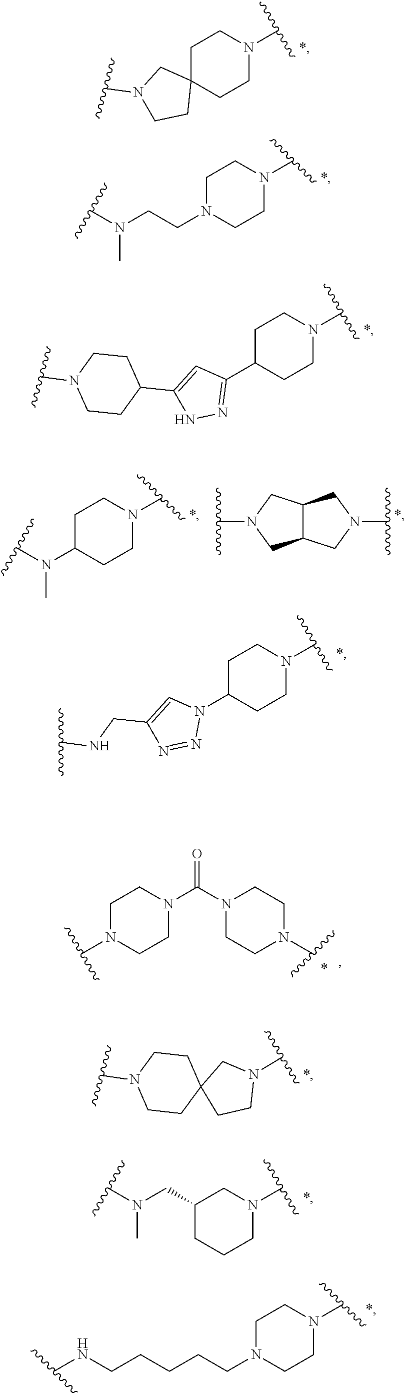

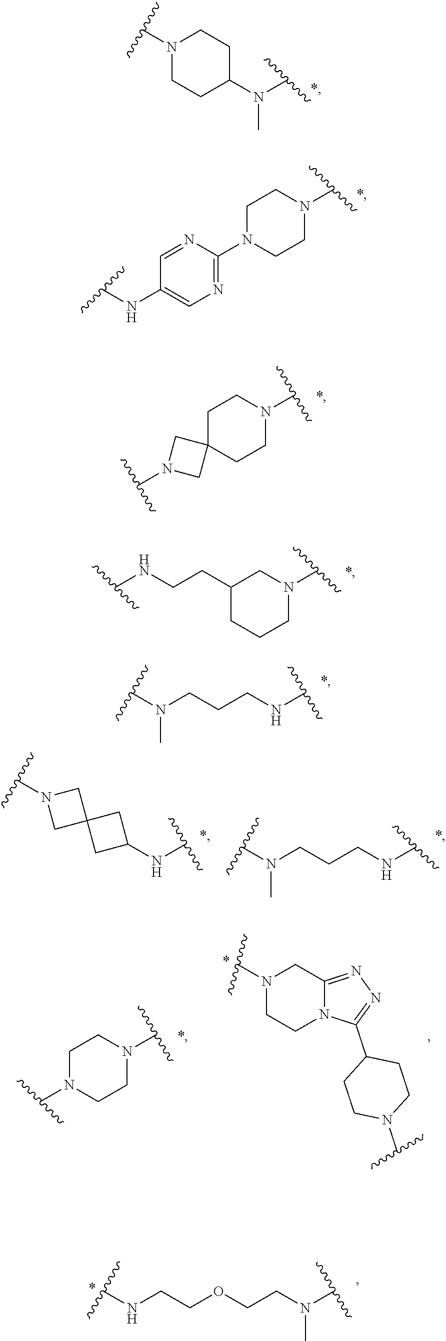

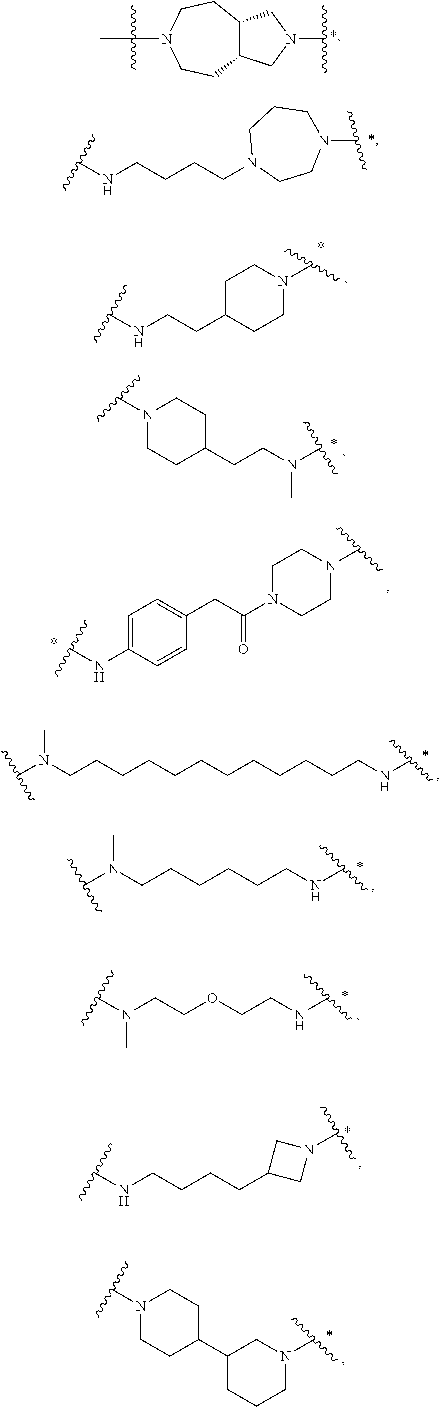

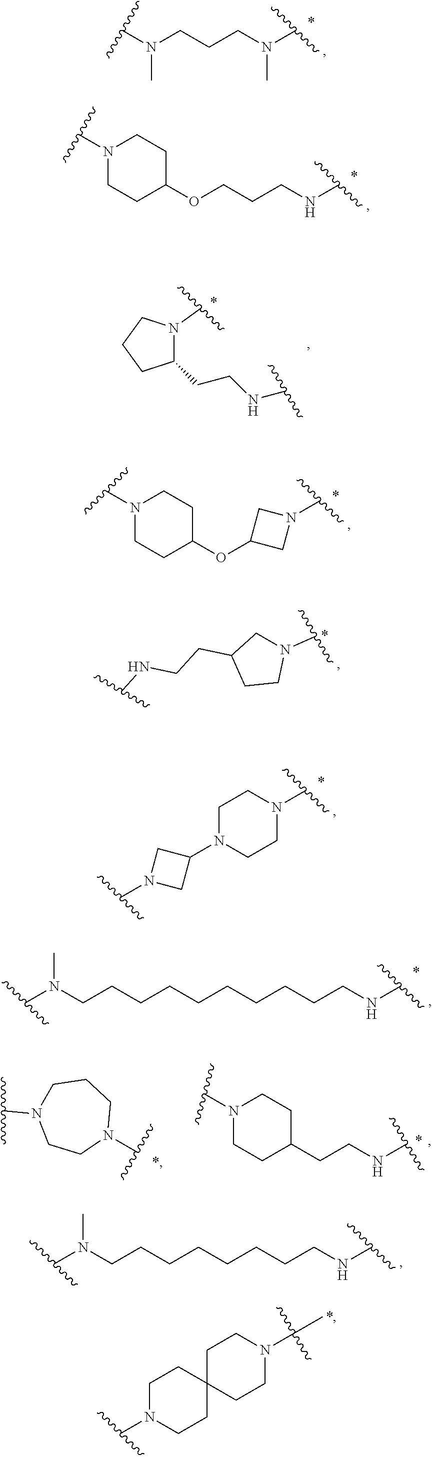

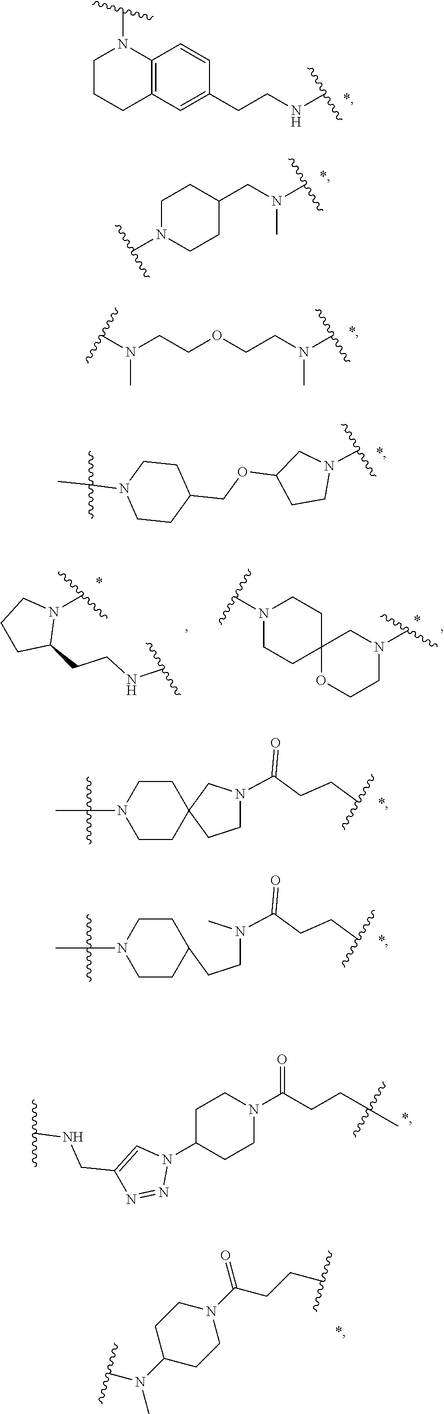

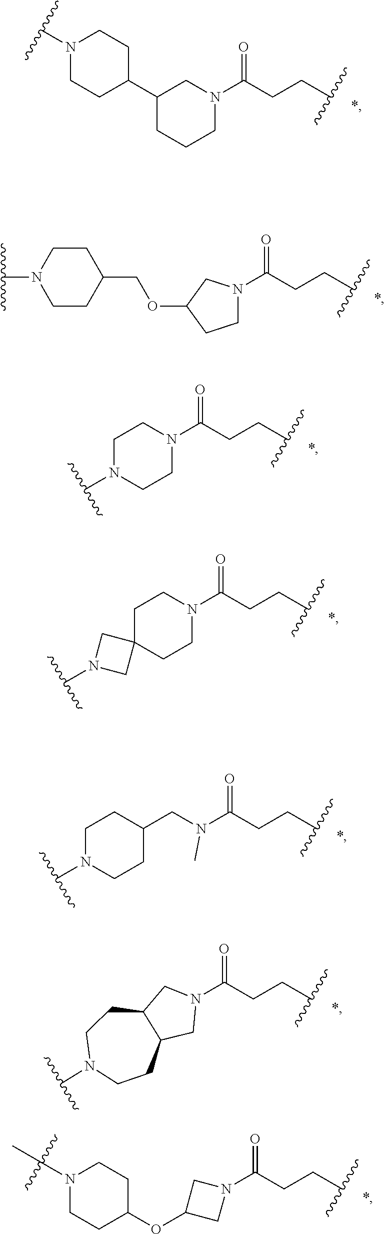

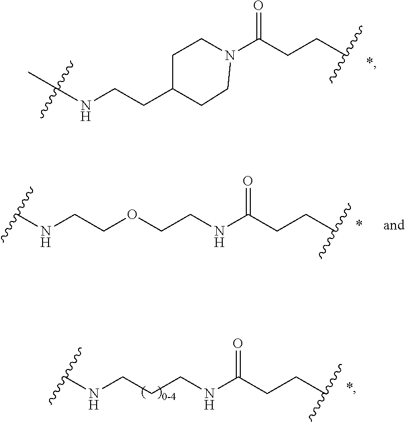

or a pharmaceutically acceptable salt, hydrate, solvate, prodrug, stereoisomer, or tautomer thereof, wherein [symbol] represents the point of attachment to the Targeting Ligand in Formula (I-a); [symbol] represents the point of attachment to the Targeting Ligase Binder in Formula (I-a).

### Targeting Ligase Binders-Linkers

In an embodiment, the targeting ligase binder and L2 have a structure of Formula (TLB-L2-I):

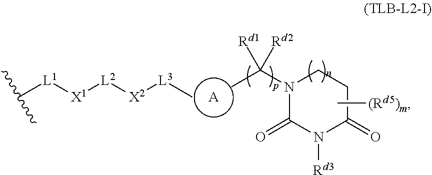

or a pharmaceutically acceptable salt, hydrate, solvate, prodrug, stereoisomer, or tautomer thereof, wherein

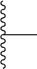

denotes the point of attachment to the targeting ligand in Formula (I-a); L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L2-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2.

In an embodiment, ring A is a 5-membered nitrogen-containing heteroaryl or a 6-membered nitrogen-containing heteroaryl (e.g., a nitrogen-containing heteroaryl). In an embodiment, ring A is a 5-membered heteroaryl. In an embodiment, A is a 5-membered nitrogen-containing heteroaryl. In an embodiment, A is a 6-membered heteroaryl. In an embodiment, ring A is a 6-membered nitrogen-containing heteroaryl. In an embodiment, ring A is selected from the group consisting of phenyl, pyridyl, pyridonyl, pyrazinyl, thiophenyl, pyrazolyl, imidazolyl, and pyrrolyl. In an embodiment, ring A is pyridyl or pyridonyl. In an embodiment, ring A is pyridyl. In an embodiment, R^{d4} is hydroxyl or C₁₋₆ alkoxyl.

In an embodiment, the targeting ligase binder and L2 have a structure selected from the group consisting of Formulas (TLB-L2-I-i), (TLB-L2-I-ii), and (TLB-L2-I-iii):

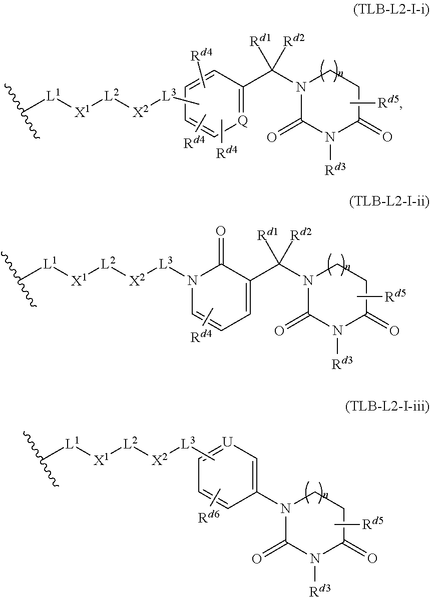

or a pharmaceutically acceptable salt, hydrate, solvate, prodrug, stereoisomer, or tautomer thereof,
wherein

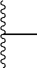

denotes the point of attachment to the targeting ligand in Formula (I-a); L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or X¹-L²-X² form a spiroheterocyclyl substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-II); wherein no more than 2 of L¹, X¹, X², L², and L³ can simultaneously be a bond; Q is N or CR^{d4}; U is —CR^{d6} or N; R^{d1} and R^{d2} are each independently selected from the group consisting of H, C₁₋₆ alkyl, C₁₋₆ haloalkyl, and C₁₋₆ heteroalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl; R^{d5} is selected from the group consisting of H, C₁₋₆ alkyl, halogen, C₁₋₆ haloalkyl, and C₁₋₆ heteroalkyl; each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; R^{p} is H or C₁₋₆ alkyl; and n is 1 or 2.

In an embodiment, R^{d1} and R^{d2} are both methyl. In an embodiment, R^{d1} and R^{d2} are both H. In an embodiment, R^{d4} is H or C₁₋₆ alkyl, e.g., methyl. In an embodiment, R^{d5} is H or C₁₋₆ alkyl, e.g., methyl.

In an embodiment, R^{d4} is H or C₁₋₆ alkyl, e.g., methyl. In an embodiment, R^{d5} is H or C₁₋₆ alkyl, e.g., methyl.

In an embodiment, R^{d1}, R^{d2}, R^{d4}, and R^{d5} are each H.

In an embodiment, n is 1. In an embodiment, R^{d3} is H. In an embodiment, R^{d3} is —CH₂OP(O)(OR^{p})₂.

In an embodiment, n is 2. In an embodiment, L³ is selected from the group consisting of —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl.

In an embodiment, the Targeting Ligase Binder-Linker has Formula (TLBL-II):

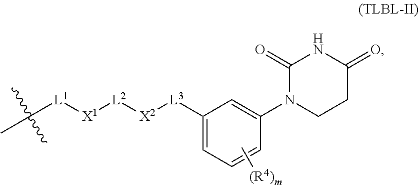

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker has Formula (TLBL-IV):

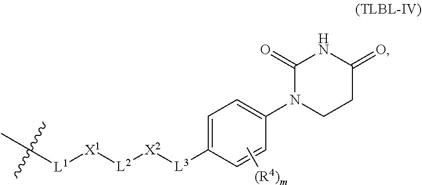

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker has Formula (TLBL-II′):

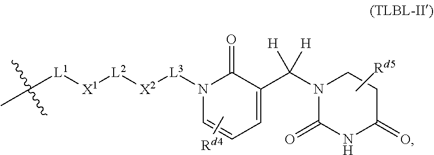

or a pharmaceutically acceptable salt, hydrate, solvate, prodrug, stereoisomer, or tautomer thereof, wherein the point of attachment to the Targeting Ligand is through L¹.

In an embodiment, the Targeting Ligase Binder-Linker has Formula (TLBL-III′):

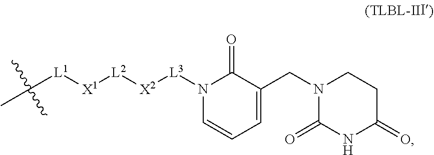

or a pharmaceutically acceptable salt, hydrate, solvate, prodrug, stereoisomer, or tautomer thereof, wherein the point of attachment to the Targeting Ligand is through L¹.

In another embodiment, the targeting ligase binder and L2, or a pharmaceutically acceptable salt, hydrate, solvate, prodrug, stereoisomer, or tautomer thereof, have a structure selected from:

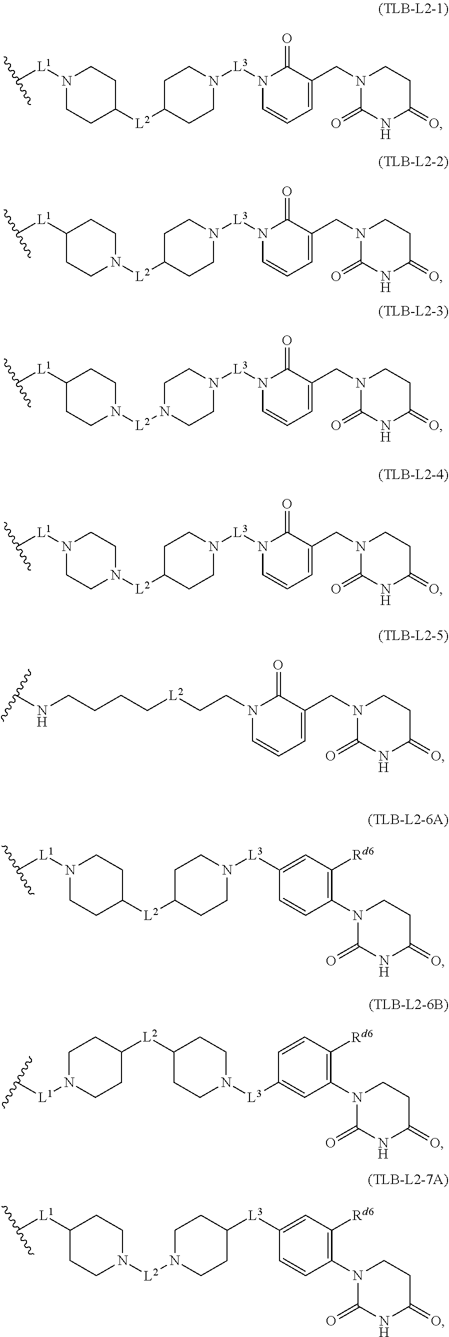

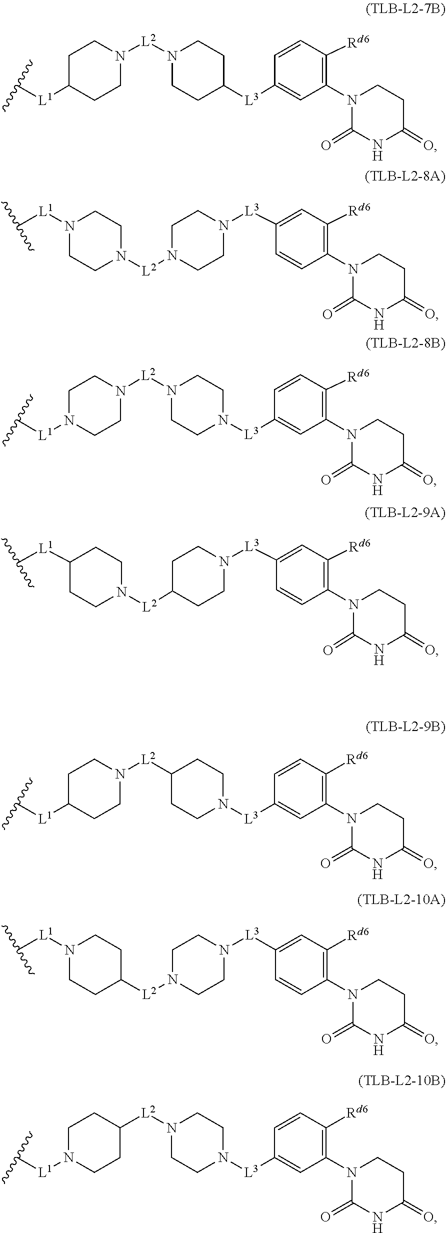

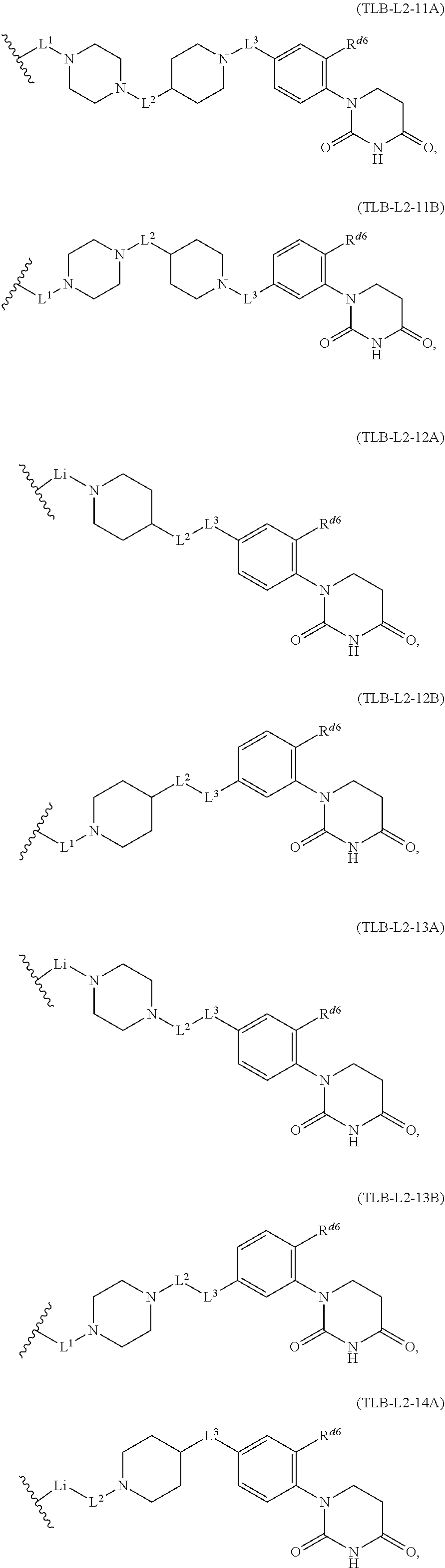

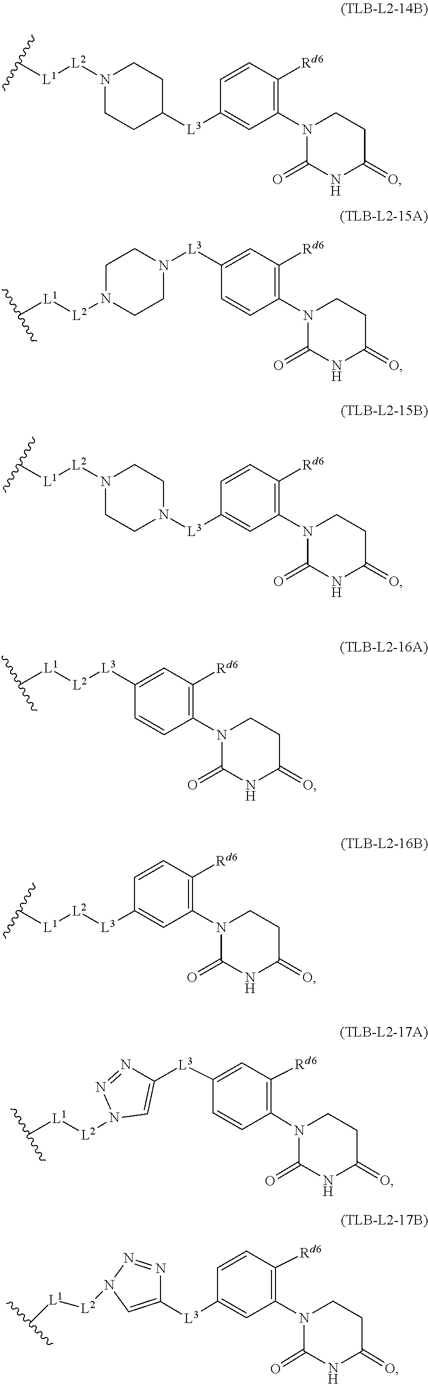

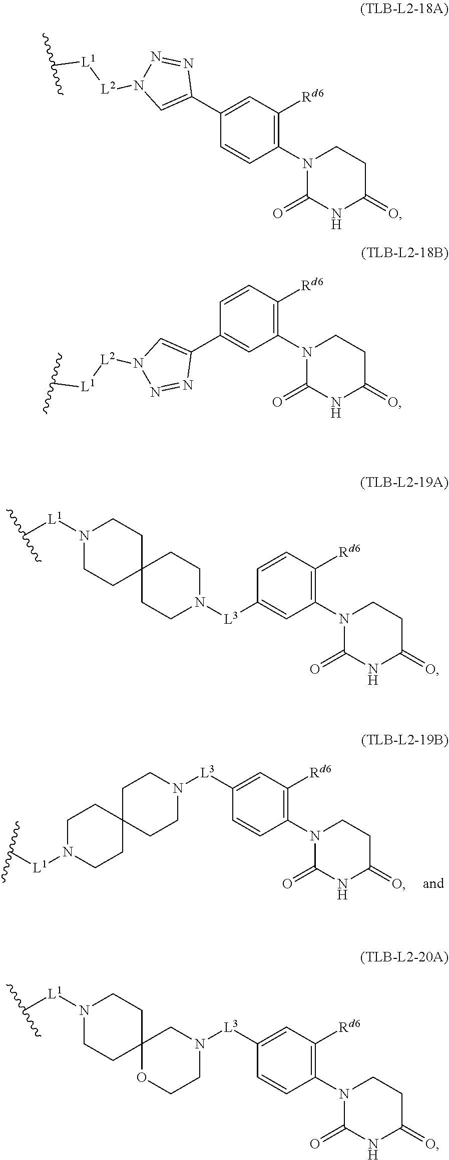

or a pharmaceutically acceptable salt, hydrate, solvate, prodrug, stereoisomer, or tautomer thereof, wherein each of L¹, L², L³, and R^{d6} is as defined herein, and

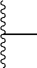

denotes the point of attachment to the targeting ligand in Formula (I-a).

In another aspect, the bifunctional protein degrader (e.g., of Formula (I-a)) has a structure of Formula (BFD-I):

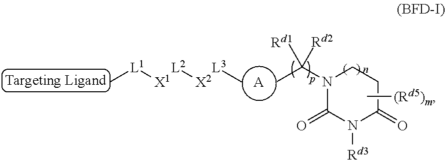

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand;
X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BFD-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2, wherein the targeting ligand is a group capable of binding to a target protein.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered heteroaryl. In an embodiment, ring A is a 5-membered nitrogen-containing heteroaryl. In an embodiment, ring A is a 6-membered heteroaryl. In an embodiment, ring A is a 6-membered nitrogen-containing heteroaryl. In an embodiment, ring A is pyridyl.

In an embodiment, n is 1. In an embodiment, n is 2. In an embodiment, R^{d3} is —CH₂OP(O)(OR^{p})₂. In an embodiment, R^{d3} is H.

In another embodiment, the bifunctional protein degrader (e.g, of Formula (I-a)) has a structure selected from the group consisting of Formulas (BFD-1-i), (BFD-I-ii), and (BFD-I-iii):

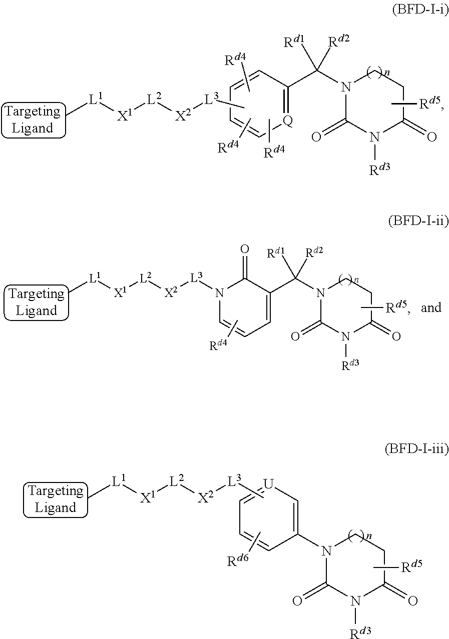

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-0, and *C(O)—C₁₋₉ heteroalkylene, wherein * denotes the point of attachment of L³ to X² in any one of Formulas (BFD-1-i), (BFD-I-ii), or (BFD-I-iii); wherein no more than 2 of L¹, X¹, X², L², and L³ can simultaneously be a bond; Q is N or CR^{d4}; U is —CR^{d6} or N; R^{d1} and R^{d2} are each independently selected from the group consisting of H, C₁₋₆ alkyl, C₁₋₆ haloalkyl, and C₁₋₆ heteroalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl; R^{d5} is selected from the group consisting of H, C₁₋₆ alkyl, halogen, C₁₋₆ haloalkyl, and C₁₋₆ heteroalkyl; each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; R^{p} is H or C₁₋₆ alkyl; and n is 1 or 2, wherein the targeting ligand is a group capable of binding to a target protein.

In an embodiment, n is 1. In an embodiment, n is 2. In an embodiment, R^{d3} is —CH₂OP(O)(OR^{p})₂. In an embodiment, R^{d3} is H.

In an embodiment, R^{d3} is H. In an embodiment, —X¹-L²-X²— is:

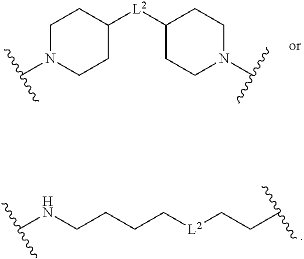

In an embodiment, L¹ is —O— or C₁₋₆ alkylene. In an embodiment, R^{d1} and R^{d2} are both methyl. In an embodiment, R^{d1} and R^{d2} are both H. In an embodiment, R^{d4} is H or C₁₋₃ alkyl. In an embodiment, R^{d5} is H or C₁₋₃ alkyl.

In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl.

In an embodiment, R^{d7} is —CH₂OP(O)(OR^{p})₂. In an embodiment, R^{d7} is H. In an embodiment, U is —CR^{d6}. In an embodiment, R^{d8} is H. In an embodiment, R^{d7} and R^{d8} are each independently H. In an embodiment, R^{d6} is H. In an embodiment, R^{d6} is selected from the group consisting of H, halogen, C₁₋₆ alkyl, and C₁₋₆ alkoxyl. In an embodiment, R^{d6} is selected from the group consisting of H, halogen, C₁₋₆ alkyl, and C₁₋₆ alkoxyl; and R^{d7}, and R^{d8} are each H.

In another embodiment, L¹-X¹-L²-X²-L³ is selected from the group consisting of:

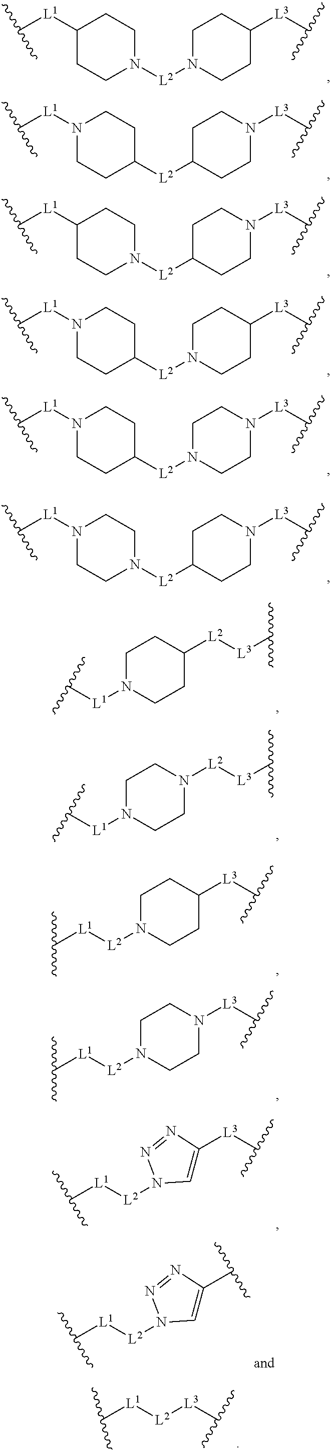

In an embodiment, L³ is selected from the group consisting of a bond, —O—, —C(O)—, —S(O)₂—, C₁₋₆ alkylene, C₂₋₆ alkynylene, and C₁₋₆ heteroalkylene.

In an embodiment, the targeting ligand is a BTK targeting ligand. In an embodiment, the targeting ligand is a BTK targeting ligand of Formula (BTK-I):

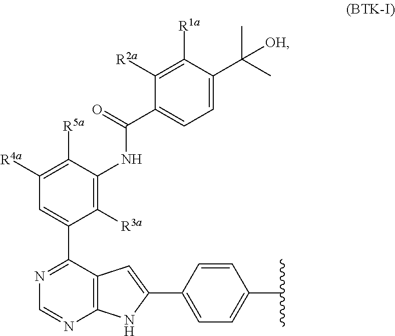

or a pharmaceutically acceptable salt, hydrate, solvate, prodrug, stereoisomer, or tautomer thereof, wherein R^{1a} is H or halo; R^{2a} is halo; R^{3a} is C₁₋₆ alkyl; R^{4a} is halo; and R^{5a} is H or halo.

In another embodiment, the bifunctional protein degrader (e.g., of Formula (I-a)) or the BTK degrader Compound has a structure of Formula (BFD-BTK-I), (BFD-BTK-II), (BFD-BTK-III), (BFD-BTK-I-a), (BFD-BTK-II-a), or (BFD-BTK-III-a):

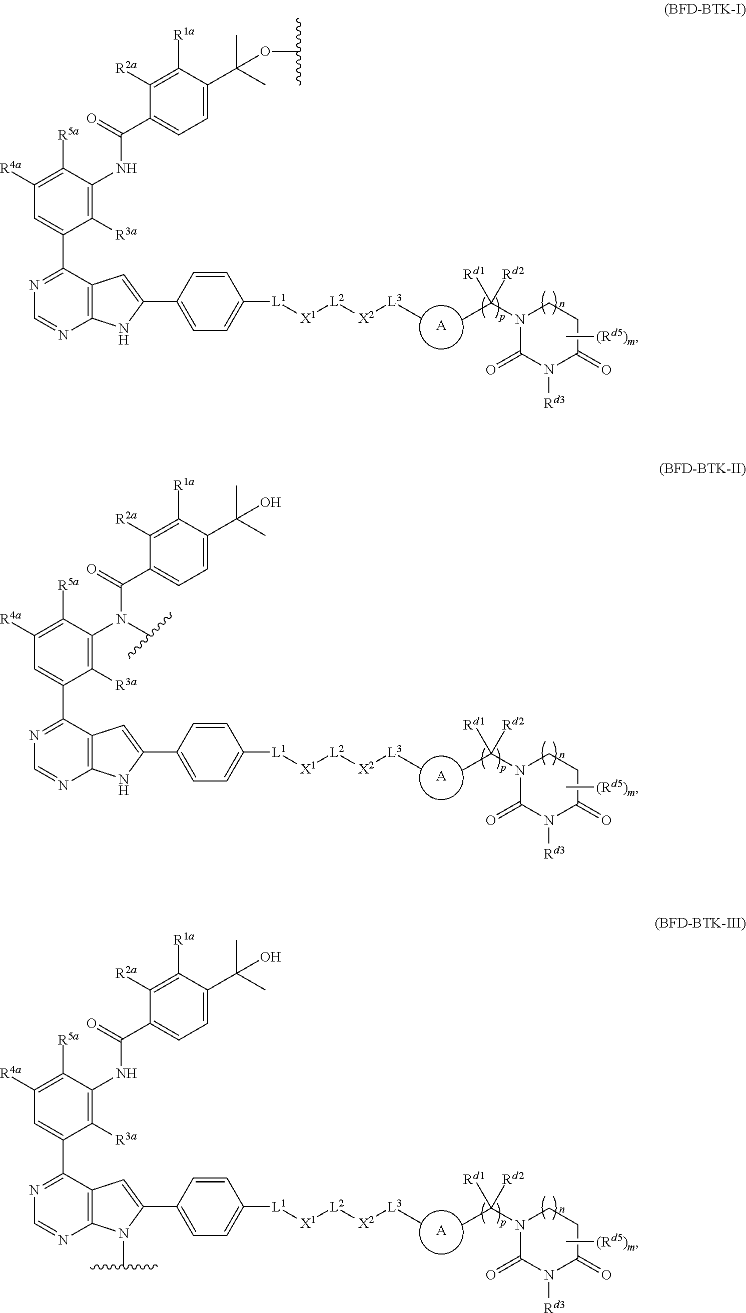

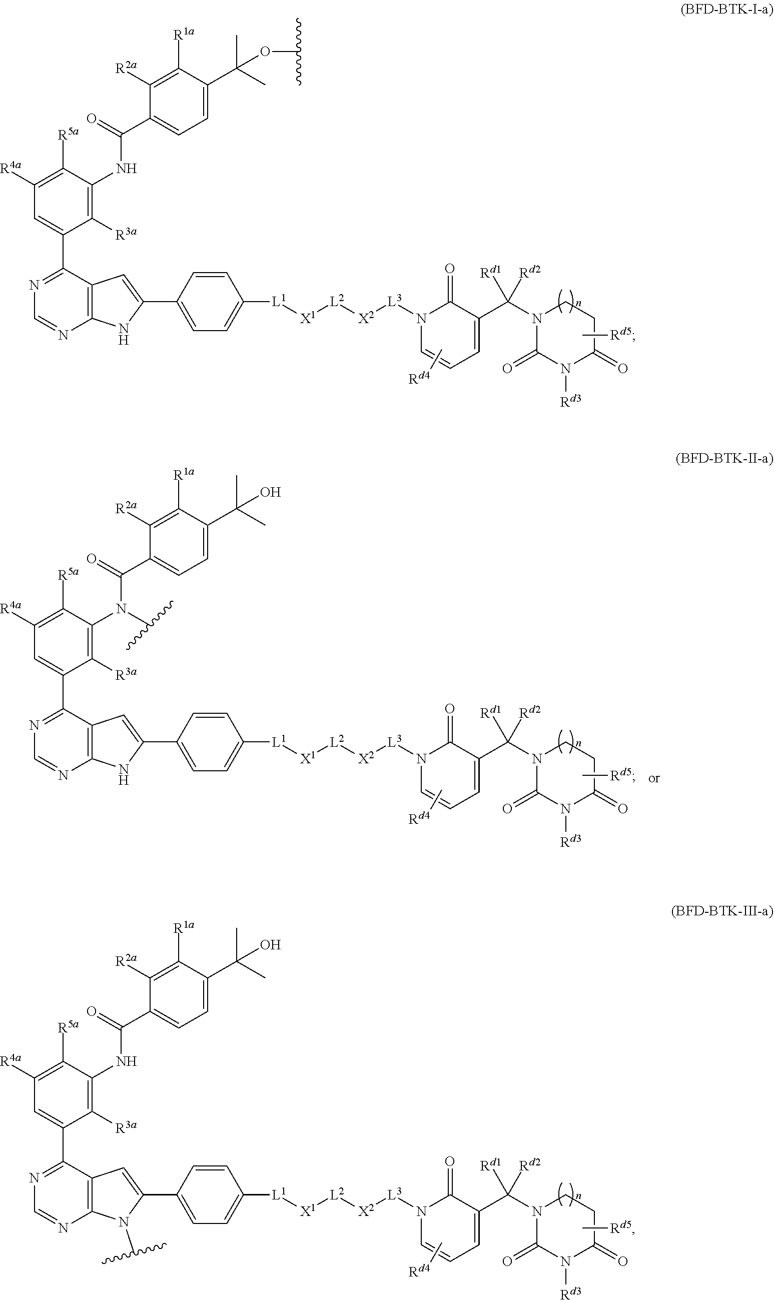

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to X¹; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-0, and *C(O)—C₁₋₉ heteroalkylene, wherein * denotes the point of attachment of L³ to X²; wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; n is 1 or 2; R^{1a} is H or halo; R^{2a} is H or halo; R^{3a} is C₁₋₆ alkyl; R^{4a} is H or halo; R^{5a} is H or halo; and

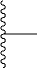

denotes the point of attachment to L1 in Formula (I) or the point of attachment to the Linker L⁴ in Formula (I) or Fatty Acid when the Linker is absent.

In an embodiment,

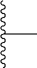

denotes the point of attachment to L1 in Formula (I). In an embodiment,

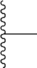

denotes the point of attachment to the Linker L⁴ or Fatty Acid when the Linker is absent in Formula (I′).

In an embodiment, the bifunctional protein degrader (e.g., of Formula (I-a)) or the BTK degrader Compound has a structure of Formula (BFD-BTK-I), (BFD-BTK-II), or (BFD-BTK-III). In an embodiment, the bifunctional protein degrader (e.g., of Formula (I-a) or (I′-a)) or the BTK degrader Compound has a structure of Formula ((BFD-BTK-I-a), (BFD-BTK-II-a), or (BFD-BTK-III-a). In an embodiment, the bifunctional protein degrader is a derivative that may form a conjugate. The other variables are as defined above.

In some embodiments, the bifunctional protein degrader or a pharmaceutically acceptable salt, hydrate, solvate, prodrug, stereoisomer, or tautomer thereof, selected from:

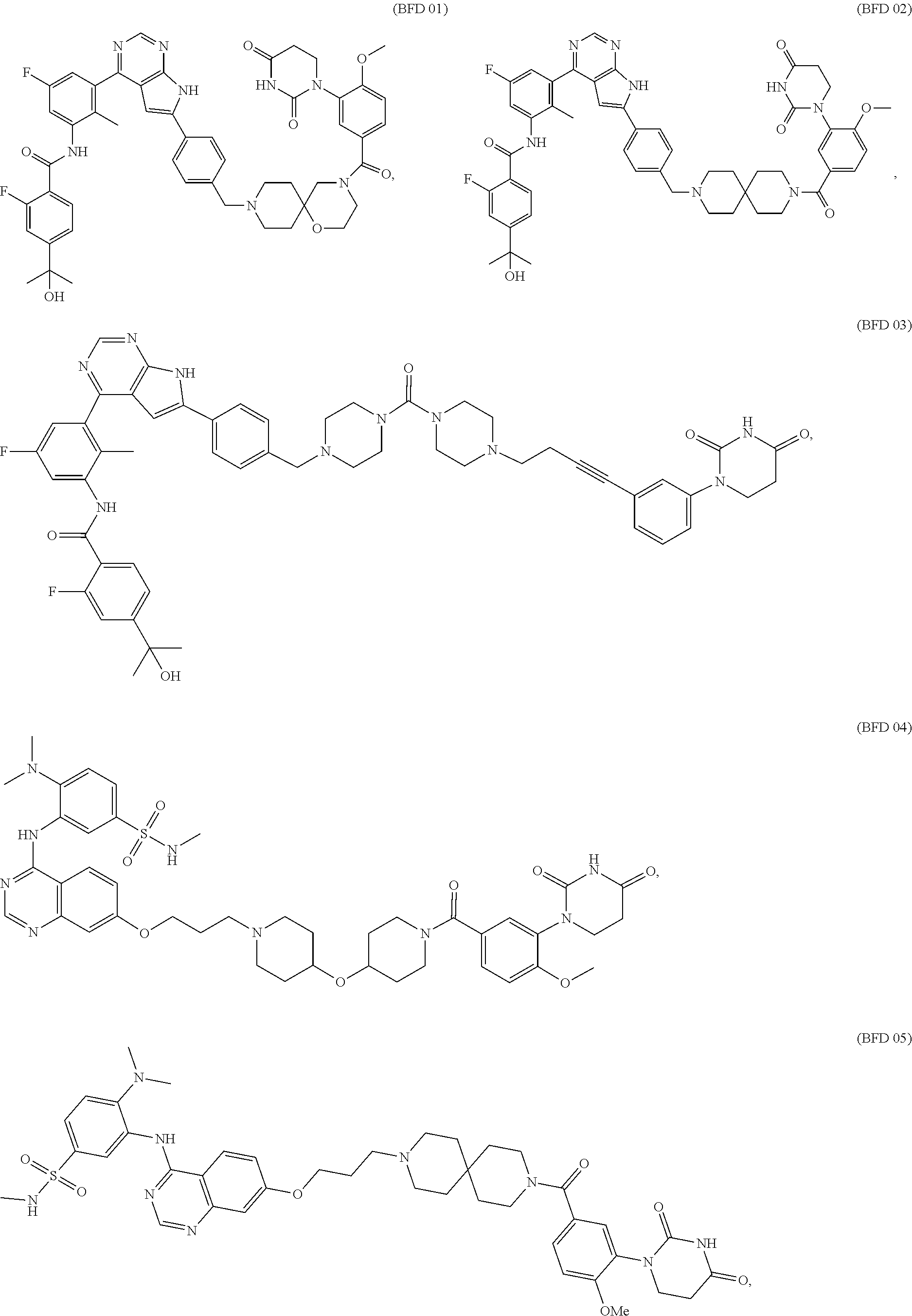

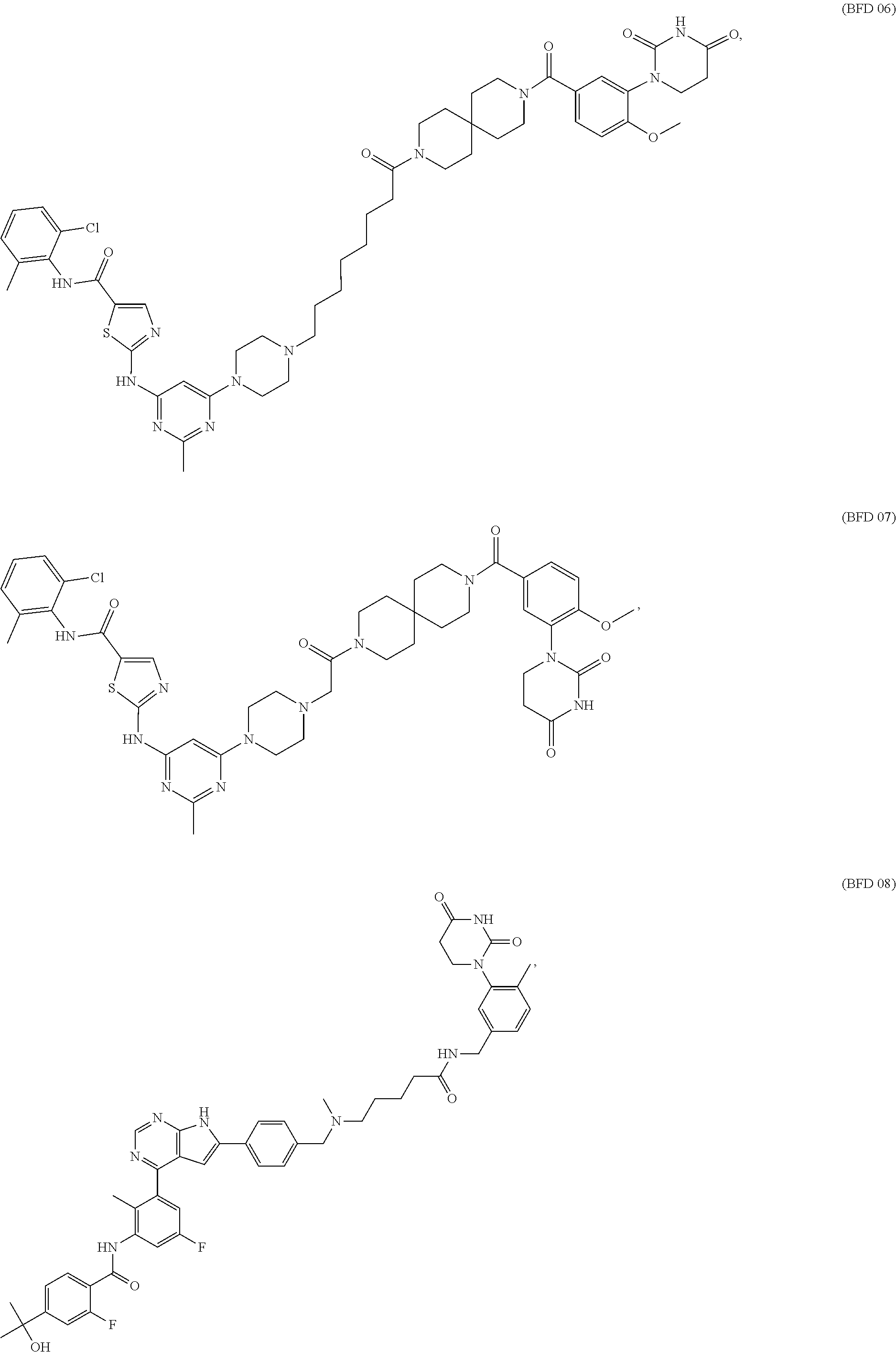

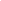

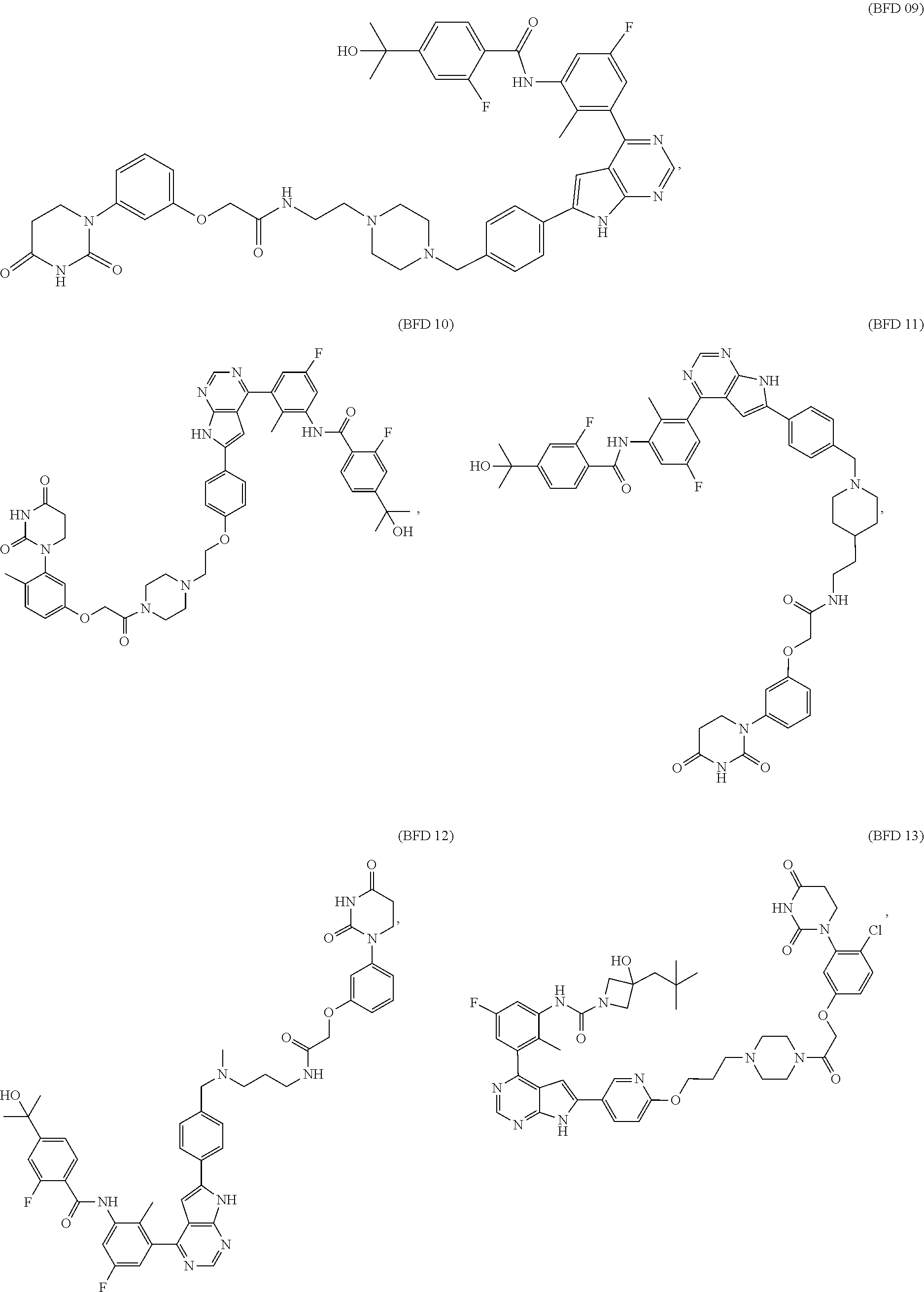

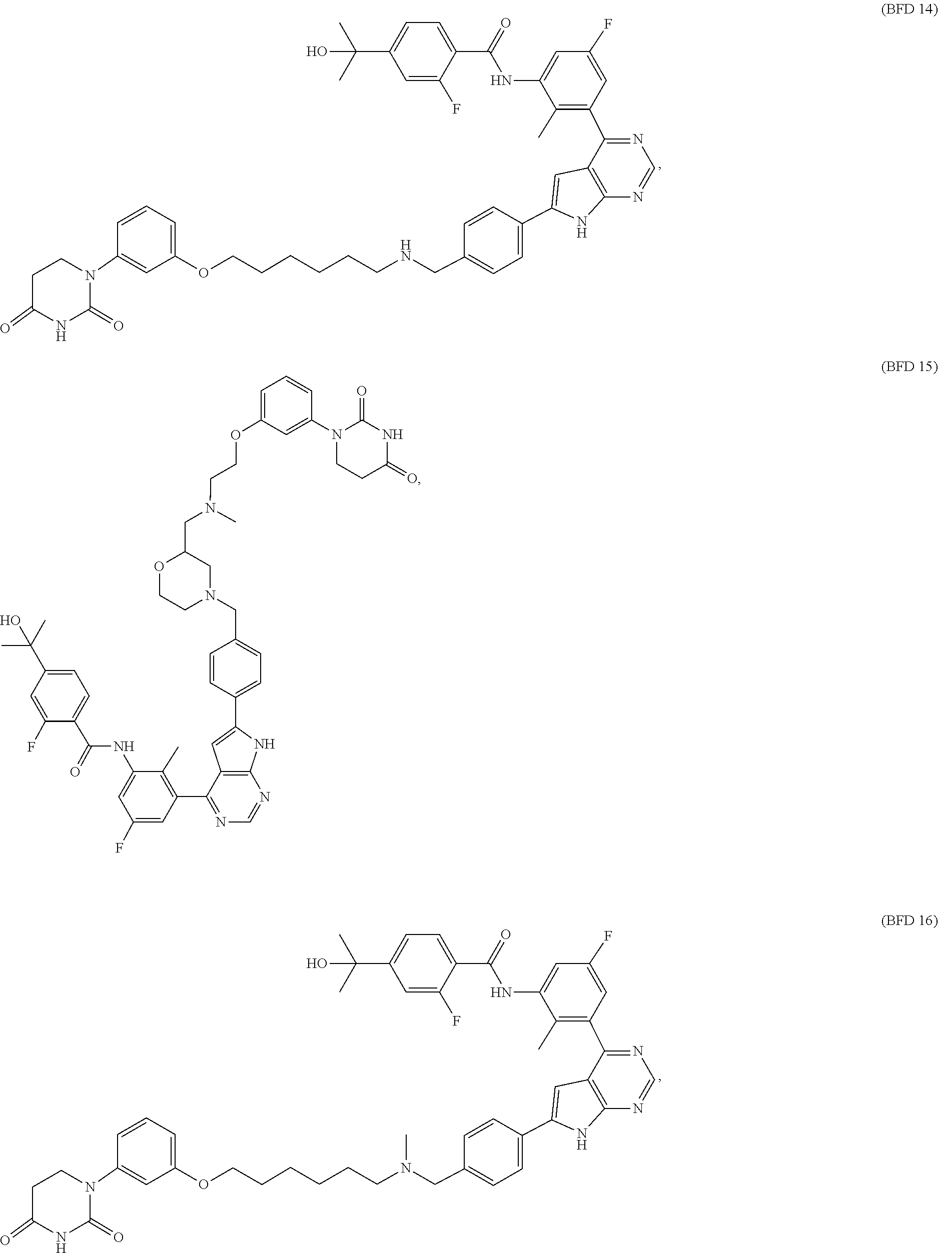 [SMILES: Cc1c(NC(=O)c2ccc(C(C)(C)O)cc2F)cc(F)cc1-c1ncnc2[nH]c(-c3ccc(CNCCCCCCOc4cccc(N5CCC(=O)NC5=O)c4)cc3)cc12 ; Cc1c(NC(=O)c2ccc(C(C)(C)O)cc2F)cc(F)cc1-c1ncnc2[nH]c(-c3ccc(CN4CCOC(CN(C)CCOc5cccc(N6CCC(=O)NC6=O)c5)C4)cc3)cc12 ; Cc1c(NC(=O)c2ccc(C(C)(C)O)cc2F)cc(F)cc1-c1ncnc2[nH]c(-c3ccc(CN(C)CCCCCCOc4cccc(N5CCC(=O)NC5=O)c4)cc3)cc12]

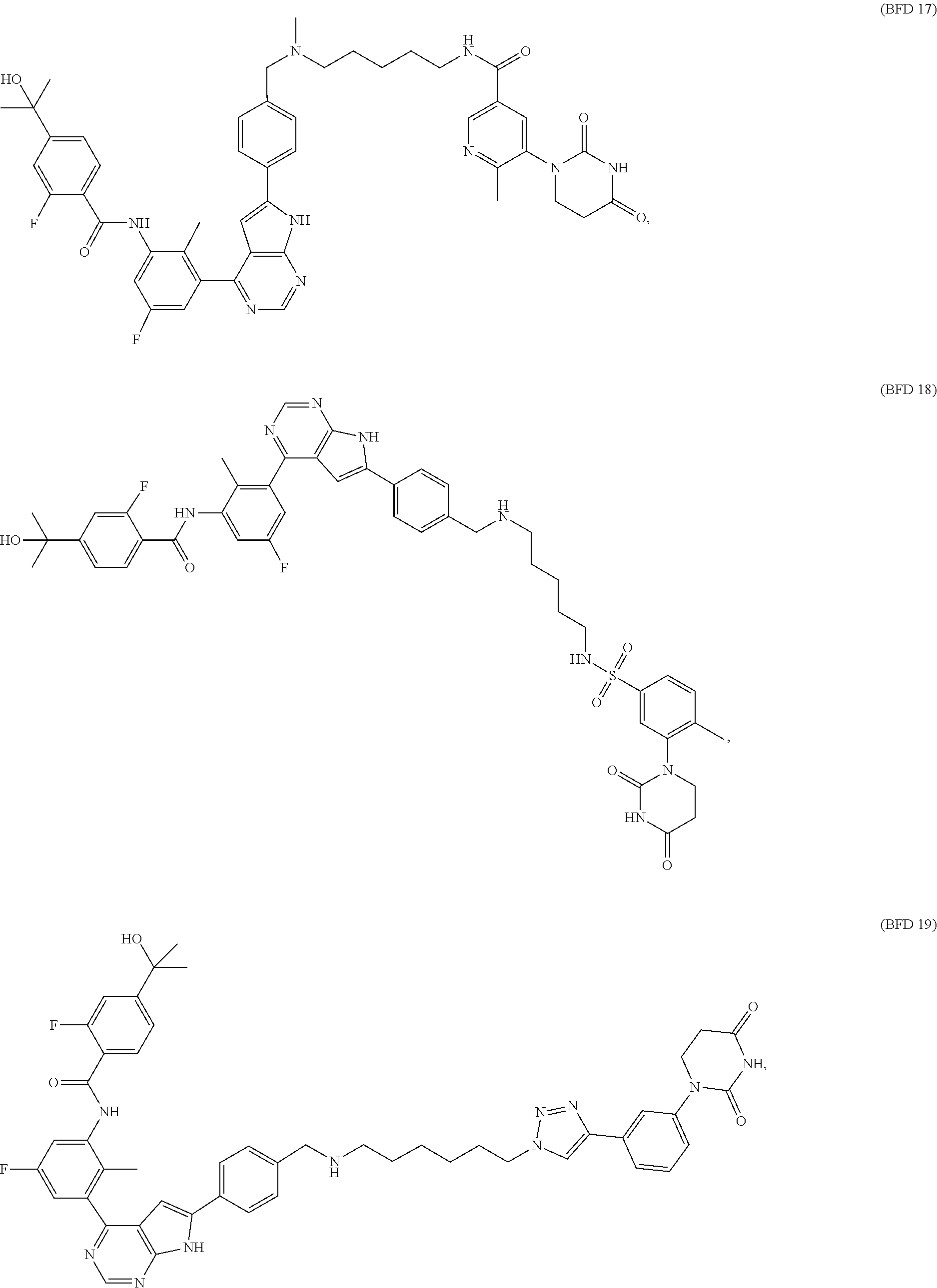 [SMILES: Cc1ncc(C(=O)NCCCCCN(C)Cc2ccc(-c3cc4c(-c5cc(F)cc(NC(=O)c6ccc(C(C)(C)O)cc6F)c5C)ncnc4[nH]3)cc2)cc1N1CCC(=O)NC1=O ; Cc1ccc(S(=O)(=O)NCCCCCNCc2ccc(-c3cc4c(-c5cc(F)cc(NC(=O)c6ccc(C(C)(C)O)cc6F)c5C)ncnc4[nH]3)cc2)cc1N1CCC(=O)NC1=O ; Cc1c(NC(=O)c2ccc(C(C)(C)O)cc2F)cc(F)cc1-c1ncnc2[nH]c(-c3ccc(CNCCCCCCn4cc(-c5cccc(N6CCC(=O)NC6=O)c5)nn4)cc3)cc12]

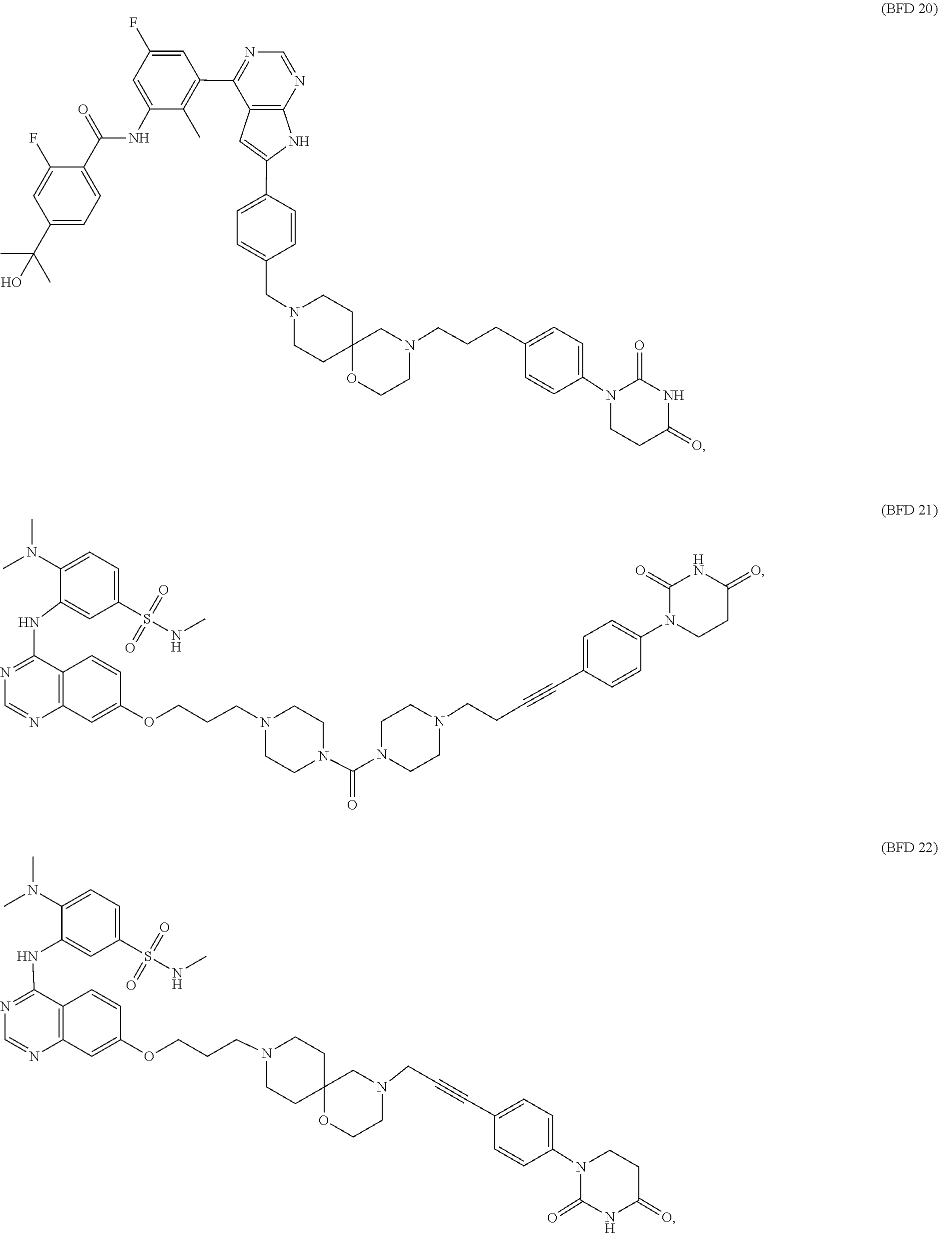 [SMILES: Cc1c(NC(=O)c2ccc(C(C)(C)O)cc2F)cc(F)cc1-c1ncnc2[nH]c(-c3ccc(CN4CCC5(CC4)CN(CCCc4ccc(N6CCC(=O)NC6=O)cc4)CCO5)cc3)cc12 ; CNS(=O)(=O)c1ccc(N(C)C)c(Nc2ncnc3cc(OCCCN4CCN(C(=O)N5CCN(CCC#Cc6ccc(N7CCC(=O)NC7=O)cc6)CC5)CC4)ccc23)c1 ; CNS(=O)(=O)c1ccc(N(C)C)c(Nc2ncnc3cc(OCCCN4CCC5(CC4)CN(CC#Cc4ccc(N6CCC(=O)NC6=O)cc4)CCO5)ccc23)c1]

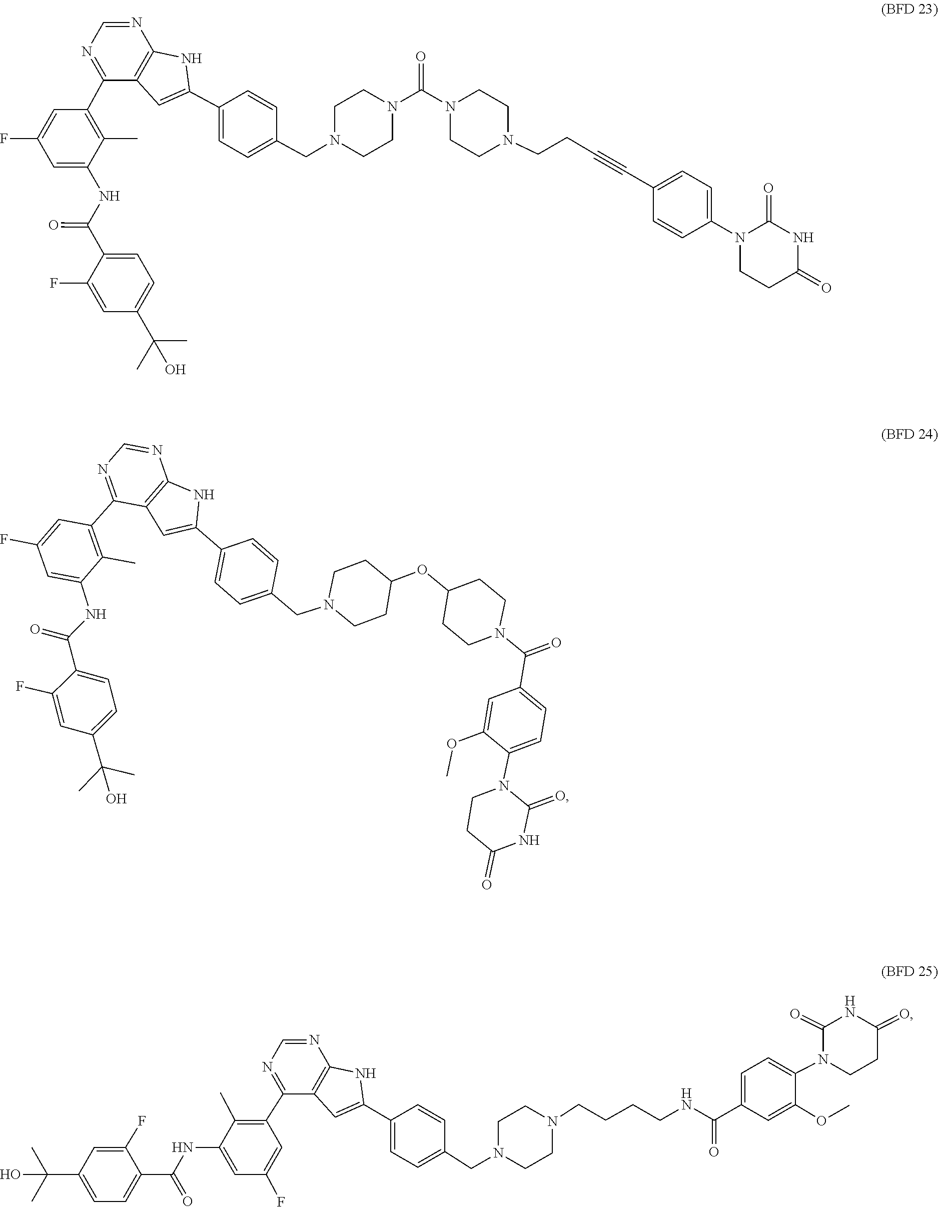 [SMILES: Cc1c(NC(=O)c2ccc(C(C)(C)O)cc2F)cc(F)cc1-c1ncnc2[nH]c(-c3ccc(CN4CCN(C(=O)N5CCN(CCC#Cc6ccc(N7CCC(=O)NC7=O)cc6)CC5)CC4)cc3)cc12 ; COc1cc(C(=O)N2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O ; COc1cc(C(=O)NCCCCN2CCN(Cc3ccc(-c4cc5c(-c6cc(F)cc(NC(=O)c7ccc(C(C)(C)O)cc7F)c6C)ncnc5[nH]4)cc3)CC2)ccc1N1CCC(=O)NC1=O]

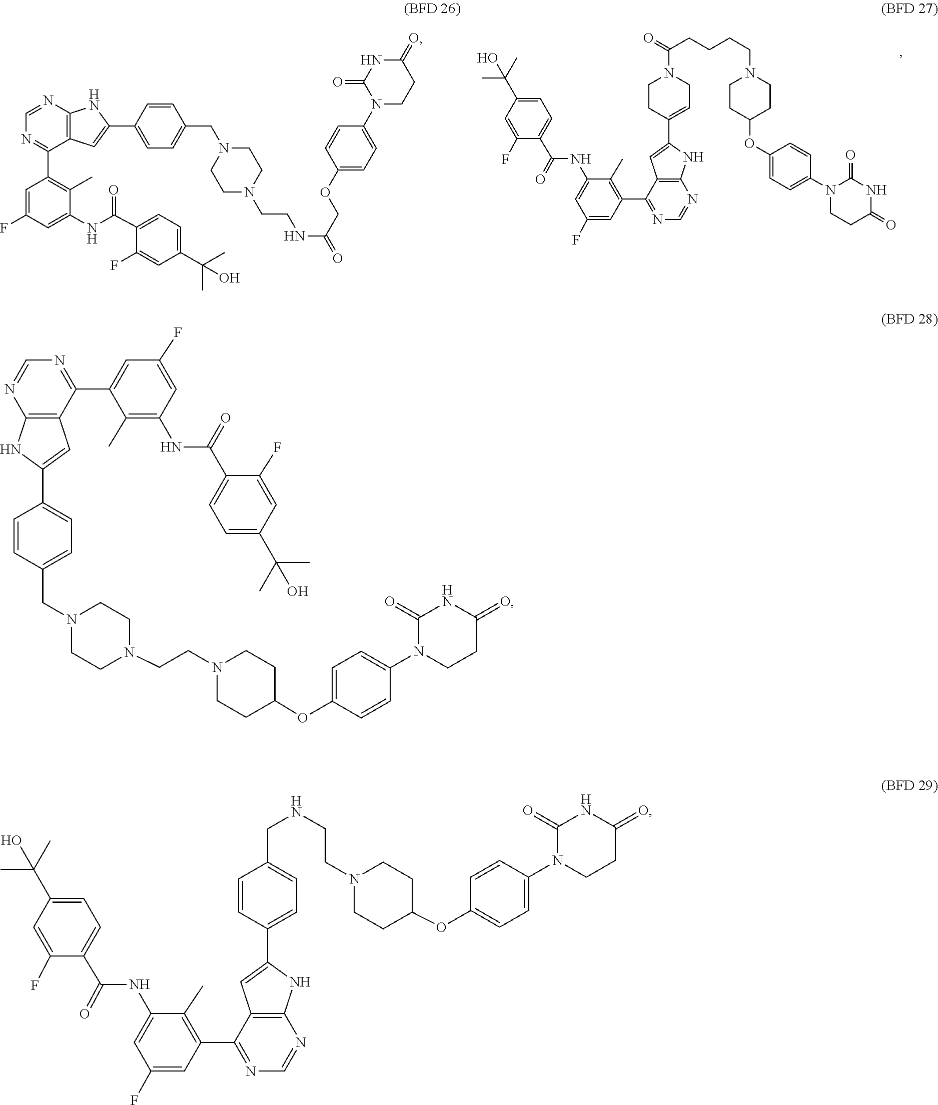 [SMILES: Cc1c(NC(=O)c2ccc(C(C)(C)O)cc2F)cc(F)cc1-c1ncnc2[nH]c(-c3ccc(CN4CCN(CCNC(=O)COc5ccc(N6CCC(=O)NC6=O)cc5)CC4)cc3)cc12 ; Cc1c(NC(=O)c2ccc(C(C)(C)O)cc2F)cc(F)cc1-c1ncnc2[nH]c(C3=CCN(C(=O)CCCCN4CCC(Oc5ccc(N6CCC(=O)NC6=O)cc5)CC4)CC3)cc12 ; Cc1c(NC(=O)c2ccc(C(C)(C)O)cc2F)cc(F)cc1-c1ncnc2[nH]c(-c3ccc(CN4CCN(CCN5CCC(Oc6ccc(N7CCC(=O)NC7=O)cc6)CC5)CC4)cc3)cc12 ; Cc1c(NC(=O)c2ccc(C(C)(C)O)cc2F)cc(F)cc1-c1ncnc2[nH]c(-c3ccc(CNCCN4CCC(Oc5ccc(N6CCC(=O)NC6=O)cc5)CC4)cc3)cc12]

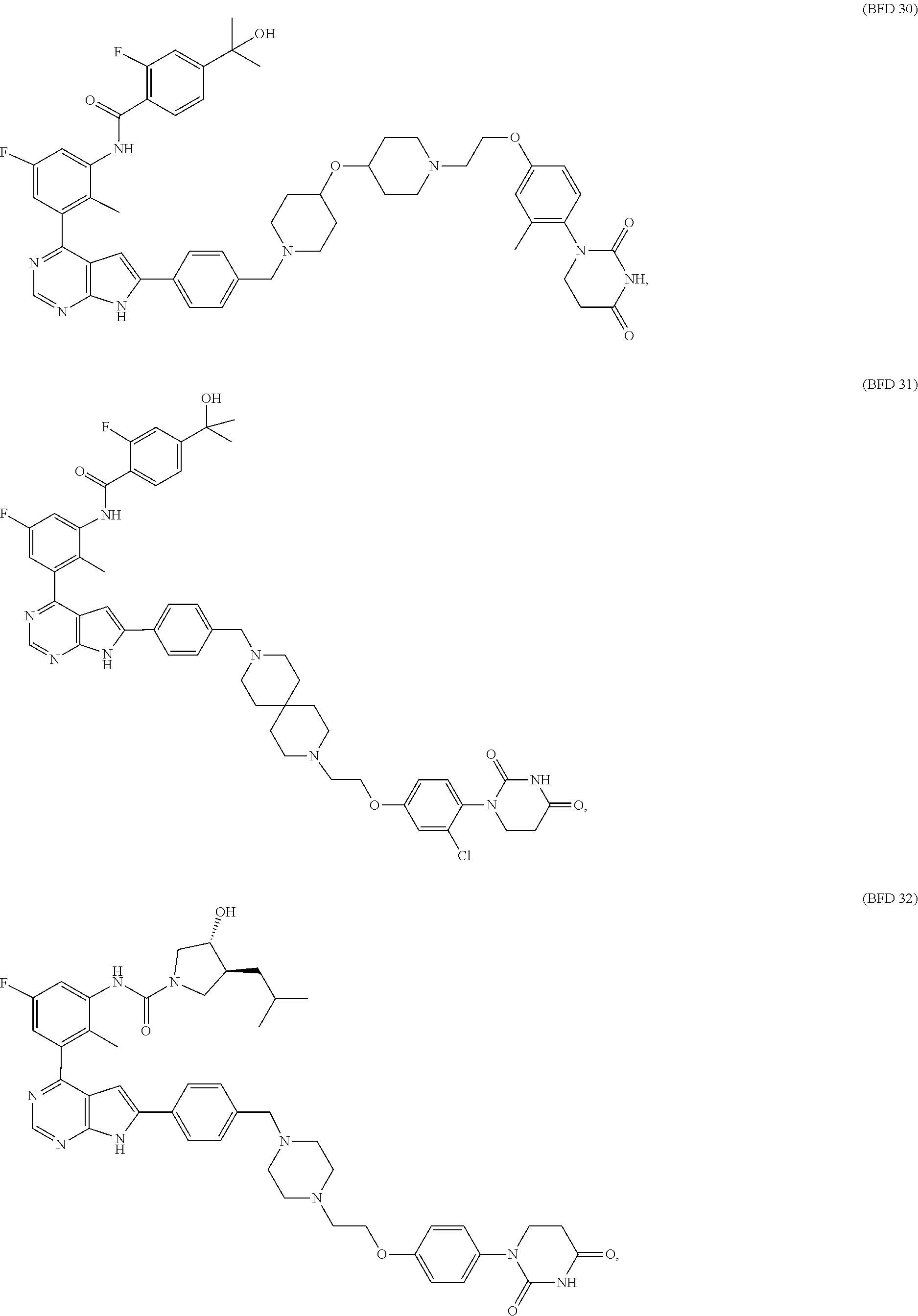 [SMILES: Cc1cc(OCCN2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O ; Cc1c(NC(=O)c2ccc(C(C)(C)O)cc2F)cc(F)cc1-c1ncnc2[nH]c(-c3ccc(CN4CCC5(CCN(CCOc6ccc(N7CCC(=O)NC7=O)c(Cl)c6)CC5)CC4)cc3)cc12 ; Cc1c(NC(=O)N2C[C@H](CC(C)C)[C@@H](O)C2)cc(F)cc1-c1ncnc2[nH]c(-c3ccc(CN4CCN(CCOc5ccc(N6CCC(=O)NC6=O)cc5)CC4)cc3)cc12]

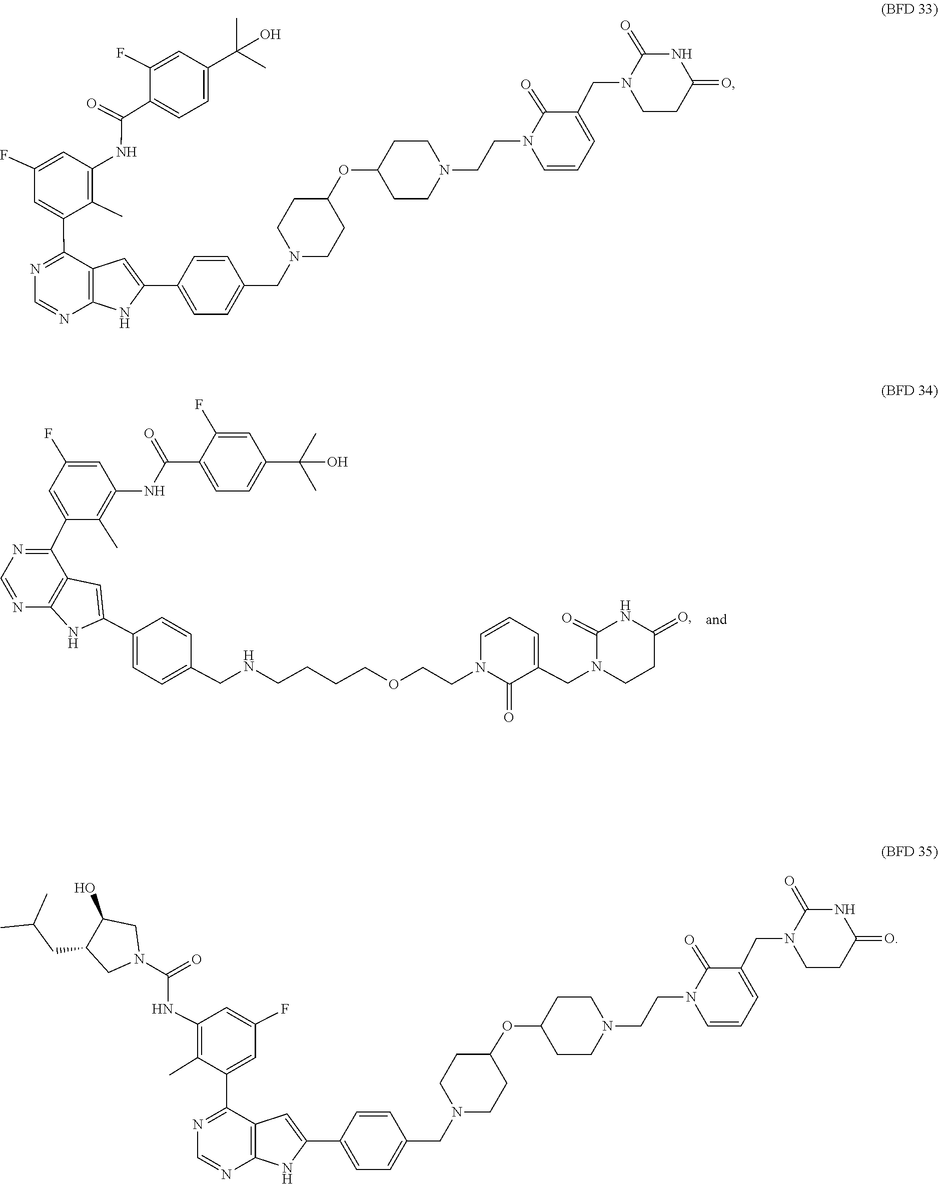 [SMILES: Cc1c(NC(=O)c2ccc(C(C)(C)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12 ; Cc1c(NC(=O)c2ccc(C(C)(C)O)cc2F)cc(F)cc1-c1ncnc2[nH]c(-c3ccc(CNCCCCOCCn4cccc(CN5CCC(=O)NC5=O)c4=O)cc3)cc12 ; Cc1c(NC(=O)N2C[C@H](CC(C)C)[C@@H](O)C2)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

In some embodiments, the bifunctional protein degrader is a derivative of BFD 01 to BFD 35 that may be covalently linked to the fatty acid via a linker.

### Cleavable Linkers of the Conjugates

In an embodiment, the Linker is absent and the Bifunctional Protein Degrader is covalently linked, i.e., directly linked through a covalent bond, to the Fatty Acid.

The present disclosure features fatty acid-bifunctional protein degrader conjugates further comprising a cleavable linker, e.g., which provides for release of the bifunctional protein degrader from the fatty acid. The presence of the cleavable group may aid the release of the degrader through hydrolytic cleavage.

In an embodiment, when present, the cleavable portion of the linker, e.g., L1 or L⁴, may comprise a natural or unnatural amino acid, e.g. an amino acid selected from the group consisting of arginine, histidine, lysine, aspartic acid, glutamic acid, serine, threonine, asparagine, glutamine, cysteine, selenocysteine, proline, glycine, alanine, valine, isoleucine, leucine, methionine, phenylalanine, tyrosine and tryptophan. In another embodiment, the cleavable linker is an amino acid selected from the group consisting of arginine, histidine, lysine, aspartic acid, glutamic acid, serine, threonine, asparagine, glutamine, cysteine, selenocysteine, proline, glycine, alanine, valine, isoleucine, leucine, methionine, phenylalanine, tyrosine and tryptophan. In another embodiment, the cleavable linker, e.g., L1 or L⁴, comprises an amino acid selected from the group consisting of glycine, alanine, valine, isoleucine and leucine. In an embodiment, the cleavable linker is glycine.

In one embodiment, the cleavable portion of the Linker L⁴ or L1 comprises 1, 2, 3, 4, or 5 natural or unnatural amino acids. In another embodiment, the cleavable portion of the Linker L⁴ or L1 comprises 1, 2, 3, or 4 natural or unnatural amino acids. In another embodiment, the cleavable portion of the Linker L⁴ or L1 comprises 1, 2, or 3 natural or unnatural amino acids. In yet another embodiment, the cleavable portion of the Linker L⁴ or L1 comprises 1 or 2 natural or unnatural amino acids. In another embodiment, the cleavable portion of the Linker L⁴ or L1 comprises 2, 3, 4, or 5 natural or unnatural amino acids. In yet another embodiment, the cleavable portion of the Linker L⁴ or L1 comprises 2, 3, or 4, natural or unnatural amino acids. In another embodiment, the cleavable portion of the Linker L⁴ or L1 comprises 3, 4, or 5 natural or unnatural amino acids. In yet another embodiment, the cleavable portion of the Linker L⁴ or L1 comprises 2 or 3 natural or unnatural amino acids. In another embodiment, the cleavable portion of the Linker L⁴ or L1 comprises 3 or 4 natural or unnatural amino acids. In yet another embodiment, the cleavable portion of the Linker L⁴ or L1 comprises 4 or 5 natural or unnatural amino acids.

In another embodiment, the cleavable portion of the Linker L⁴ or L1 comprises 1, 2, 3, 4, or 5 natural amino acids. In another embodiment, the cleavable portion of the Linker L⁴ or L1 comprises 1, 2, 3, or 4 natural amino acids. In another embodiment, the cleavable portion of the Linker L⁴ or L1 comprises 1, 2, or 3 natural amino acids. In yet another embodiment, the cleavable portion of the Linker L⁴ or L1 comprises 1 or 2 natural amino acids. In another embodiment, the cleavable portion of the Linker L⁴ or L1 comprises 2, 3, 4, or 5 natural amino acids. In yet another embodiment, the cleavable portion of the Linker L⁴ or L1 comprises 2, 3, or 4, natural amino acids. In another embodiment, the cleavable portion of the Linker L⁴ or L1 comprises 3, 4, or 5 natural amino acids. In yet another embodiment, the cleavable portion of the Linker L⁴ or L1 comprises 2 or 3 natural amino acids. In another embodiment, the cleavable portion of the Linker L⁴ or L1 comprises 3 or 4 natural amino acids. In yet another embodiment, the cleavable portion of the Linker L⁴ or L1 comprises 4 or 5 natural amino acids.

In yet another embodiment, the cleavable portion of the Linker L⁴ or L1 comprises 1, 2, 3, 4, or 5 unnatural amino acids. In another embodiment, the cleavable portion of the Linker L⁴ or L1 comprises 1, 2, 3, or 4 unnatural amino acids. In another embodiment, the cleavable portion of the Linker L⁴ or L1 comprises 1, 2, or 3 unnatural amino acids. In yet another embodiment, the cleavable portion of the Linker L⁴ or L1 comprises 1 or 2 unnatural amino acids. In another embodiment, the cleavable portion of the Linker L⁴ or L1 comprises 2, 3, 4, or 5 unnatural amino acids. In yet another embodiment, the cleavable portion of the Linker L⁴ or L1 comprises 2, 3, or 4, unnatural amino acids. In another embodiment, the cleavable portion of the Linker L⁴ or L1 comprises 3, 4, or 5 unnatural amino acids. In yet another embodiment, the cleavable portion of the Linker L⁴ or L1 comprises 2 or 3 unnatural amino acids. In another embodiment, the cleavable portion of the Linker L⁴ or L1 comprises 3 or 4 unnatural amino acids. In yet another embodiment, the cleavable portion of the Linker L⁴ or L1 comprises 4 or 5 unnatural amino acids.

In an embodiment, the conjugate of Formula (I) or (I′) comprises a cleavable linker L1 or L⁴ that connects the bifunctional protein degrader, and when present, the solubilizing domain. In an embodiment, when the solubilizing domain is not present, L1 or L⁴ directly connects the bifunctional protein degrader to the fatty acid. In an embodiment, the cleavable linker L1 or L⁴ is (e.g., directly) covalently linked to the bifunctional protein degrader. In an embodiment, the cleavable linker L1 is (e.g., directly) covalently linked to the solubilizing domain, when present. In an embodiment, the cleavable linker L⁴ comprises a solubilizing domain, when present. In an embodiment, the cleavable linker L1 or L⁴ is covalently linked to both the bifunctional protein degrader and the solubilizing domain, when present. In an embodiment, the cleavable linker L1 or L⁴ is covalently linked to both the bifunctional protein degrader and the fatty acid. This may aid the release of the degrader.

In one embodiment, a cleavable portion of the Linker L⁴ or L1 is directly attached to the Bifunctional Protein Degrader, e.g., via the terminal oxygen of the Bifunctional Protein Degrader, and optionally a solubilizing portion of the Linker L⁴ or a solubilizing domain is directly attached to the Fatty Acid. In another embodiment, a cleavable portion of the Linker L⁴ or L1 is directly attached to the Bifunctional Protein Degrader, e.g., via the amide nitrogen of the Bifunctional Protein Degrader, and optionally a solubilizing portion of the Linker L⁴ or a solubilizing domain is directly attached to the Fatty Acid. In another embodiment, a cleavable portion of the Linker L⁴ or L1 is directly attached to the Bifunctional Protein Degrader, e.g., via the pyrrolopyrimidine nitrogen of the Bifunctional Protein Degrader, and optionally a solubilizing portion of the Linker L⁴ or a solubilizing domain is directly attached to the Fatty Acid.

In an embodiment, the Linker L1 or L⁴ comprises a hydrophilic moiety. In an embodiment, the Linker L1 or L⁴ comprises a PEG (polyethylene glycol) moiety. In an embodiment, the Linker L1 or L⁴ comprises a PEG moiety of formula

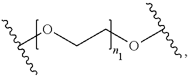

where n1 is from 1 to 35, e.g. 5 to 30, e.g. 6 to 25, e.g. 6 to 20, e.g. 7 to 15, e.g. 9 to 13, or e.g. 7, 11 or 23.

The cleavable linker L1 or L⁴ may be degraded or hydrolyzed at physiological conditions. In some embodiments, L1 or L⁴ comprises a bond cleavable in a cell (e.g, a cell organelle) or the serum, e.g., of a sample or subject. For example, L1 or L⁴ may be pH sensitive (e.g., acid labile or base labile) or cleaved through the action of an enzyme. In an embodiment, the rate of hydrolysis of L1 or L⁴ is increased by at least 0.5 fold (e.g., at least 1, 1.5, 2, 2.5, 3, 4, 5, 7.5, 10, 12.5, 15, 20, 25, 50, 75, 100, 250, 500, 750, 1000 or more) compared with the rate of hydrolysis of L1 or L⁴, respectively, in the absence of an enzyme. In an embodiments, the enzyme is an esterase.

The rate of cleavage of the cleavable portion of the linker or rate of release of the Bifunctional Protein Degrader may be affected by the size and electronic nature of the cleavable portion of the linker and/or the released Bifunctional Protein Degrader.

In an embodiment, the cleavable portion of the Linker L1 or L⁴ comprises an ester, phosphate, disulfide, thiol, hydrazone, ether, or amide. In an embodiment, the cleavable portion of the Linker (e.g., L1) is attached to the solubilizing portion, when present, of the Linker via an ester, phosphate, disulfide, thiol, hydrazone, ether, or amide. In an embodiment, the cleavable portion of the Linker L1 or L⁴ comprises an ester. In an embodiment, the cleavable portion of the Linker (e.g., L1) is attached to the solubilizing portion, when present, of the Linker via an ester. In an embodiment, the cleavable portion of the Linker L1 or L⁴ comprises an amide. In an embodiment, the cleavable portion of the Linker (e.g., L1) is attached to the solubilizing portion, when present, of the Linker via an amide.

When present, the solubilizing portion of the Linker may be attached to the cleavable portion of the Linker (e.g., L1) via a natural or unnatural amino acid through an amino group on the natural or unnatural amino acid, and a carboxyl group on the solubilizing portion. In one embodiment, the solubilizing portion (e.g., L1), when present, of the Linker is attached to the cleavable portion of the Linker via a natural or unnatural amino acid through an amino group on the natural or unnatural amino acid, and a carboxyl group on the solubilizing portion. In another embodiment, the solubilizing portion (e.g., L1), when present, of the Linker is attached to the cleavable portion of the Linker via a natural amino acid through an amino group on the natural amino acid, and a carboxyl group on the solubilizing portion. In yet another embodiment, the solubilizing portion (e.g., L1), when present, of the Linker is attached to the cleavable portion of the Linker via an unnatural amino acid through an amino group on the unnatural amino acid, and a carboxyl group on the solubilizing portion.

In an embodiment, L1 or L⁴ has the structure of Formula (L1-I) or (L1-II):

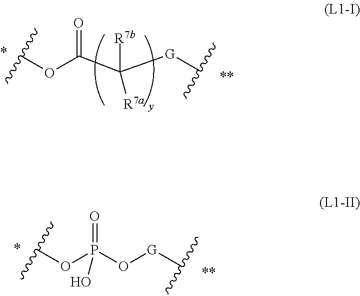

or a pharmaceutically acceptable salt, hydrate, solvate, prodrug, stereoisomer, or tautomer thereof, wherein each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, —NR′—, wherein R′ is H, C₁₋₆ alkyl, —(CH₂)₁₋₂—C(O)₂H, one or more natural or unnatural amino acids, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; and each “*” and “**” independently denote the point of attachment to the bifunctional protein degrader or solubilizing domain, when present, or fatty acid in L1 or L⁴ in Formula (I) or (I′).

In one embodiment, each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, —NR′— wherein R′ is H, C₁₋₆ alkyl, or —(CH₂)₁₋₂—C(O)₂H, 1 to 5 natural or unnatural amino acids, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; and each “*” and “**” independently denote the point of attachment to the bifunctional protein degrader or solubilizing domain, when present, or fatty acid in L1 or L⁴ in Formula (I) or (I′).

In another embodiment, each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; and each “*” and “**” independently denote the point of attachment to the bifunctional protein degrader or solubilizing domain, when present, or fatty acid in L1 or L⁴ in Formula (I) or (I′). In an embodiment, when the solubilizing domain is not present, then each and “**” independently denote the point of attachment to the bifunctional protein degrader and the fatty acid.

In an embodiment, L1 or L⁴ is selected from the group consisting of:

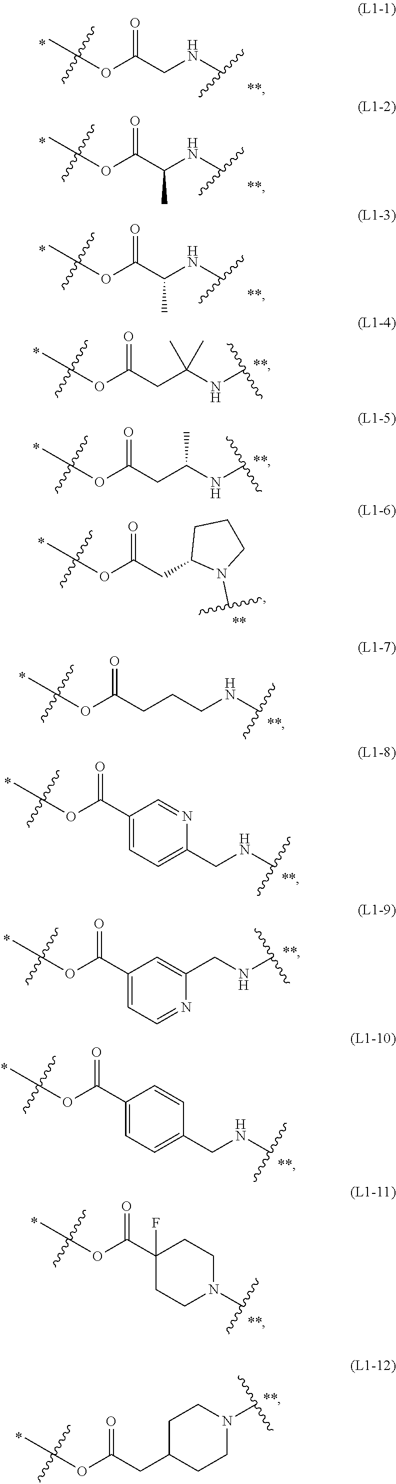

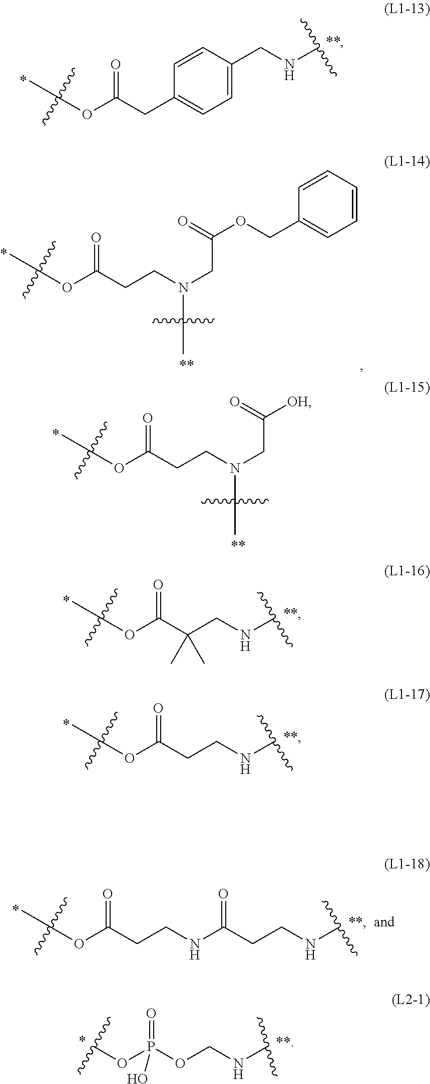

In an embodiment, the bifunctional protein degrader and L1 or L⁴ have the structure of Formula (BFD-L1-I):

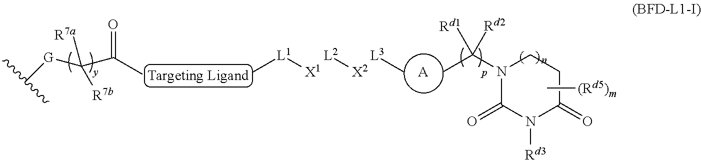

or a pharmaceutically acceptable salt, hydrate, solvate, prodrug, stereoisomer, or tautomer thereof, wherein each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, —NR′— wherein R′ is H, C₁₋₆ alkyl, or —(CH₂)₁₋₂—C(O)₂H, 1 to 5 natural or unnatural amino acids, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y 0, 1, 2, 3, 4, or 5; L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BFD-L1-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d8} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2, wherein the targeting ligand is a group capable of binding to a target protein, and

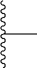

denotes the point of attachment to the solubilizing domain, when present, or fatty acid in Formula (I) or (I′).

In another embodiment, the bifunctional protein degrader and L1 or L⁴ have the structure of Formula (BFD-L1-II):

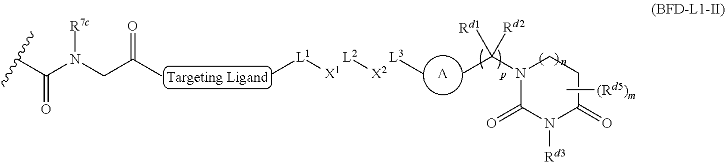

or a pharmaceutically acceptable salt, hydrate, solvate, prodrug, stereoisomer, or tautomer thereof, wherein R^{7c} is H or C₁₋₆ alkyl; L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-0, and *C(O)—C₁₋₉ heteroalkylene, wherein * denotes the point of attachment of L³ to X² in Formula (BFD-L1-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2, wherein the targeting ligand is a group capable of binding to a target protein, and

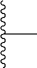

denotes the point of attachment to the solubilizing domain, when present, or fatty acid in Formula (I) or (I′).

In such BTK Degrader Compounds of Formula (BFD-BTK-II) or (BFD-BTK-III) and (BFD-BTK-II-a) or (BFD-BTK-III-a), the linker and Fatty Acid are as described in relation to compounds of Formula (BFD-BTK-I) and Formula (BFD-BTK-I-a), respectively, except in that the cleavable portion of the linker may also be

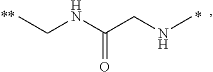

wherein ** indicates the point of attachment to the BTK degrader, and * indicates the point of attachment to the solubilizing portion, when present, of the linker.

### Solubilizing Domains

The present disclosure features fatty acid-bifunctional protein degrader conjugates optionally comprising a solubilizing domain, e.g., which provides for flexibility and/or improved aqueous solubility of the conjugate. In an embodiment, the conjugate of Formula (I), (I′) and subformula thereof comprises a solubilizing domain, when present, that comprises a water-soluble monomer or polymer. In an embodiment, the solubilizing domain, when present, increases one or more of amphiphilicity, hydrophilicity, water-solubility, pH sensitivity, or stability of the conjugate of Formula (I) or (I′), e.g., compared to a conjugate that does not comprise the solubilizing domain. In an embodiment, the cleavable portion of the linker is absent, and the solubilizing domain is cleavable to allow release of the Bifunctional Protein Degrader.

In an embodiment, the Linker L⁴ of Formula (I′) comprises L1 and, optionally, a solubilizing domain.

In another embodiment, the cleavable portion of the Linker L⁴ or L1 is attached to the terminal oxygen atom of the Bifunctional Protein Degrader through an ester linkage, and the solubilizing portion of the Linker L⁴ or the solubilizing domain is attached to the Fatty Acid through an amide linkage. In yet another embodiment, the cleavable portion of the Linker L⁴ or L1 is attached to the amide nitrogen atom of the Bifunctional Protein Degrader through an ester linkage, and the solubilizing portion of the Linker L⁴ or the solubilizing domain is attached to the Fatty Acid through an amide linkage. In another embodiment, the cleavable portion of the Linker L⁴ or L1 is attached to the pyrrolopyrimidine nitrogen atom of the Bifunctional Protein Degrader through an ester linkage, and the solubilizing portion of the Linker L⁴ or the solubilizing domain is attached to the Fatty Acid through an amide linkage.

In one embodiment, the solubilizing portion of the Linker L⁴ or the solubilizing domain comprises C₅-C₁₀₀ alkylene, C₅-C₁₀₀ alkenylene, C₅-C₁₀₀ heteroalkylene, C₅-C₁₀₀ haloalkylene or polyethylene glycol, or one or more natural or unnatural amino acids (e.g. a polypeptide chain, e.g. a polypeptide chain comprising from 1 to 100 amino acids, e.g. 3 to 100 amino acids, e.g. 5 to 50 amino acids), or a combination thereof.

In an embodiment, the solubilizing domain, when present, comprises a hydrophilic moiety. In an embodiment, the solubilizing domain, when present, comprises a polyalkylene or polyheteroalkylene moiety. In an embodiment, the solubilizing domain, when present, comprises a polyethylene glycol (PEG), a polyethylene oxide (PEO), a polypropylene glycol (PPG), a polyglycerol (PG), a poloxamine (POX), a polybutylene oxide (PBO), polylactic acid (PLA), polyglycolic acid (PGA), poly(lactic-co-glycolic acid) (PLGA), polycaprolactone (PCL), polydioxanone (PDO), a polyanhydride, a polyacrylide, a polyvinyl, or a polyorthoester. In an embodiment, the solubilizing domain, when present, comprises a polyethylene glycol (PEG).

In an embodiment, the solubilizing domain, when present, is between 100 Da and 20,000 Da in size. In an embodiment, the solubilizing domain, when present, is between 200 Da and 1,000 Da in size. In an embodiment, the solubilizing domain, when present, comprises a PEG between 100 Da and 20,000 Da in size. In an embodiment, the solubilizing domain, when present, comprises a PEG between 200 Da and 1,000 Da in size.

In an embodiment, the solubilizing domain, when present, comprises a PEG moiety of formula

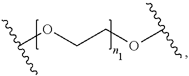

where n1 is from 1 to 35, e.g. 5 to 30, e.g. 6 to 25, e.g. 6 to 20, e.g. 7 to 15, e.g. 9 to 13, or e.g. 7, 11 or 23.

In an embodiment, the solubilizing domain, when present, comprises a PEG2, PEG3, PEG4, PEG5, PEG6, PEG7, PEG8, PEG9, PEG10, PEG11, PEG12, PEG13, PEG14, PEG15, PEG16, PEG17, PEG18, PEG19, PEG20, PEG21, PEG22, PEG23, PEG24, PEG25, PEG26, PEG27, PEG28, PEG29, or PEG30. In an embodiment, the solubilizing domain, when present, is selected from PEG2, PEG4, PEG6, PEG8, PEG10, PEG12, PEG16, PEG18, and PEG24.

In an embodiment, the solubilizing domain, when present, has a structure selected from the group consisting of Formulas (SD-I), (SD-II), and (SD-III):

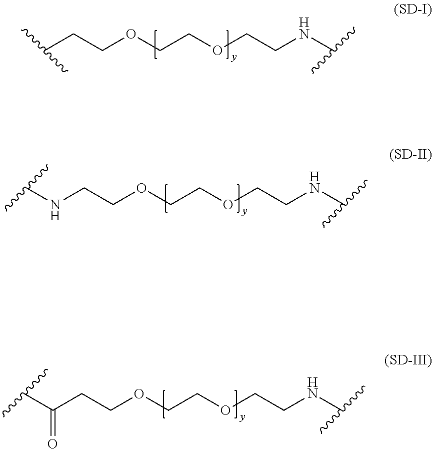

wherein y is an integer between 0 to 35; and

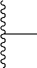

denotes the points of attachment to L1, L⁴, or the bifunctional protein degrader and the fatty acid in Formula (I) or (I′). It should be understood that, in this embodiment of the disclosure, the moiety can be placed in either orientation. For example, where the moiety is

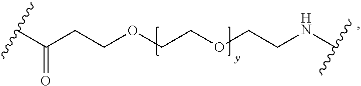

the Fatty Acid could be bonded to the carbonyl group, or the NH group. In one embodiment, the Fatty Acid is bonded to the carbonyl group of the linker. In another embodiment, the Fatty Acid is bonded to the NH group of the linker.

In another embodiment, the solubilizing portion of the Linker L⁴ or the solubilizing domain comprises a moiety having one of the following formulae:

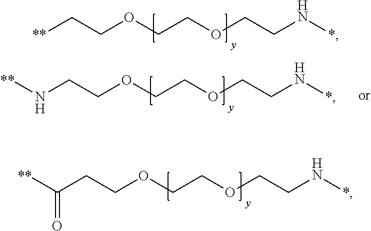

wherein y is 0 to 35, * indicates the point of attachment to the Fatty Acid, and ** indicates the point of attachment to the cleavable portion of the Linker L⁴ or L1. In one embodiment, y is 1 to 35, e.g. 5 to 30, e.g., 6 to 25, e.g. 6 to 20, e.g., 7 to 15, e.g., 9 to 13, or e.g., 11.

In an embodiment, the solubilizing domain, when present, has the structure of Formula (SD-I):

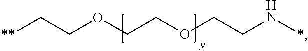

wherein * indicates the point of attachment to the fatty acid, ** indicates the point of attachment to L1 or the bifunctional protein degrader, and y is 11.

In another embodiment, the solubilizing domain, when present, comprises C₅-C₁₀₀ alkylene, C₅-C₁₀₀ alkenylene, C₅-C₁₀₀ heteroalkylene, C₅-C₁₀₀ haloalkylene or polyethylene glycol, or one or more natural or unnatural amino acids (e.g. a polypeptide chain, e.g. a polypeptide chain comprising from 1 to 100 amino acids, e.g. 3 to 100 amino acids, e.g. 5 to 50 amino acids), or a combination thereof.

In an embodiment, the solubilizing domain, when present, is an amino acid selected from the group consisting of arginine, histidine, lysine, aspartic acid, glutamic acid, serine, threonine, asparagine, glutamine, cysteine, selenocysteine, proline, glycine, alanine, valine, isoleucine, leucine, methionine, phenylalanine, tyrosine and tryptophan. In another embodiment, the solubilizing domain of the linker comprises an amino acid selected from the group consisting of glycine, alanine, valine, isoleucine and leucine. In an embodiment, the solubilizing domain of the linker is glycine.

When present, the solubilizing portion of the Linker may be attached to the cleavable portion of the Linker via a natural or unnatural amino acid through an amino group on the natural or unnatural amino acid, and a carboxyl group on the solubilizing portion. In one embodiment, the solubilizing portion, when present, of the Linker is attached to the cleavable portion of the Linker via a natural or unnatural amino acid through an amino group on the natural or unnatural amino acid, and a carboxyl group on the solubilizing portion. In another embodiment, the solubilizing portion, when present, of the Linker is attached to the cleavable portion of the Linker via a natural amino acid through an amino group on the natural amino acid, and a carboxyl group on the solubilizing portion. In yet another embodiment, the solubilizing portion, when present, of the Linker is attached to the cleavable portion of the Linker via an unnatural amino acid through an amino group on the unnatural amino acid, and a carboxyl group on the solubilizing portion.

In one embodiment, the cleavable portion of the Linker L⁴ or L1 comprises 1, 2, 3, 4, or 5 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 1, 2, 3, or 4 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 1, 2, or 3 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In yet another embodiment, the cleavable portion of the Linker L⁴ or L1 comprises 1 or 2 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 2, 3, 4, or 5 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid.

In yet another embodiment, the cleavable portion of the Linker L⁴ or L1 comprises 2, 3, or 4, natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 3, 4, or 5 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In yet another embodiment, the cleavable portion of the Linker L⁴ or L1 comprises 2 or 3 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid.

In another embodiment, the cleavable portion of the Linker L⁴ or L1 comprises 3 or 4 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In yet another embodiment, the cleavable portion of the Linker L⁴ or L1 comprises 4 or 5 natural or unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid.

In another embodiment, the cleavable portion of the Linker L⁴ or L1 comprises 1, 2, 3, 4, or 5 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 1, 2, 3, or 4 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 1, 2, or 3 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid.

In yet another embodiment, the cleavable portion of the Linker L⁴ or L1 comprises 1 or 2 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 2, 3, 4, or 5 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In yet another embodiment, the cleavable portion of the Linker L⁴ or L1 comprises 2, 3, or 4, natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 3, 4, or 5 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid.

In yet another embodiment, the cleavable portion of the Linker L⁴ or L1 comprises 2 or 3 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 3 or 4 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In yet another embodiment, the cleavable portion of the Linker L⁴ or L1 comprises 4 or 5 natural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid.

In yet another embodiment, the cleavable portion of the Linker L⁴ or L1 comprises 1, 2, 3, 4, or 5 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 1, 2, 3, or 4 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 1, 2, or 3 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid.

In yet another embodiment, the cleavable portion of the Linker L⁴ or L1 comprises 1 or 2 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 2, 3, 4, or 5 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In yet another embodiment, the cleavable portion of the Linker L⁴ or L1 comprises 2, 3, or 4, unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid.

In another embodiment, the cleavable portion of the Linker L⁴ or L1 comprises 3, 4, or 5 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In yet another embodiment, the cleavable portion of the Linker L⁴ or L1 comprises 2 or 3 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In another embodiment, the cleavable portion of the Linker L⁴ or L1 comprises 3 or 4 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In yet another embodiment, the cleavable portion of the Linker L⁴ or L1 comprises 4 or 5 unnatural amino acids which are attached to the terminal oxygen atom, the amide nitrogen atom, or the pyrrolopyrimidine nitrogen atom of the Bifunctional Protein Degrader through an ester linkage formed between the terminal oxygen and the carboxylic acid of the first amino acid, and the solubilizing portion of the Linker L⁴ or the solubilizing domain is attached to the Fatty Acid through an amide linkage to the last amino acid. In one embodiment, the cleavable portion of the Linker L⁴ or L1 comprises a natural or unnatural amino acid which is attached to the terminal oxygen atom, the amide nitrogen atom, or the pyrrolopyrimidine nitrogen atom of the Bifunctional Protein Degrader through an ester linkage formed between the terminal oxygen and the carboxylic acid of the amino acid, and the solubilizing portion of the Linker L⁴ or the solubilizing domain is attached to the Fatty Acid through an amide linkage.

In another embodiment, the cleavable portion of the Linker L⁴ or L1 comprises a natural amino acid which is attached to the terminal oxygen atom, the amide nitrogen atom, or the pyrrolopyrimidine nitrogen atom of the Bifunctional Protein Degrader through an ester linkage formed between the terminal oxygen and the carboxylic acid of the amino acid and the solubilizing portion of the Linker L⁴ or the solubilizing domain is attached to the Fatty Acid through an amide linkage. In another embodiment, the cleavable portion of the Linker L⁴ or L1 comprises an unnatural amino acid which is attached to the terminal oxygen atom, the amide nitrogen atom, or the pyrrolopyrimidine nitrogen atom of the Bifunctional Protein Degrader through an ester linkage formed between the terminal oxygen and the carboxylic acid of the amino acid, and the solubilizing portion of the Linker L⁴ or the solubilizing domain is attached to the Fatty Acid through an amide linkage.

In one embodiment, the cleavable portion of the Linker L⁴ or L1 comprises a natural or unnatural amino acid which is attached to the terminal oxygen atom of the Bifunctional Protein Degrader through an ester linkage formed between the terminal oxygen and the carboxylic acid of the amino acid, and the solubilizing portion of the Linker L⁴ or the solubilizing domain is attached to the Fatty Acid through an amide linkage.

In another embodiment, the cleavable portion of the Linker L⁴ or L1 comprises a natural amino acid which is attached to the terminal oxygen atom of the Bifunctional Protein Degrader through an ester linkage formed between the terminal oxygen and the carboxylic acid of the amino acid and the solubilizing portion of the Linker L⁴ or the solubilizing domain is attached to the Fatty Acid through an amide linkage. In another embodiment, the cleavable portion of the Linker L⁴ or L1 comprises an unnatural amino acid which is attached to the terminal oxygen atom of the Bifunctional Protein Degrader through an ester linkage formed between the terminal oxygen and the carboxylic acid of the amino acid, and the solubilizing portion of the Linker L⁴ or the solubilizing domain is attached to the Fatty Acid through an amide linkage.

In one embodiment, the cleavable portion of the Linker L⁴ or L1 is —C(O)[C(Rₐ)₂]ₓN(R_{b})—, wherein each Rₐ is independently selected from the group consisting of H and C₁-C₃ alkyl, x is 1 or 2, and R_{b} is selected from the group consisting of H and C₁-C₃ alkyl. In this embodiment of the disclosure, the cleavable portion of the Linker L⁴ or L1 can be placed in either orientation within the molecule, for example with the carbonyl group being directly attached to the bifunctional protein degrader, or with the carbonyl group being directly attached to the solubilizing portion of the linker. In one embodiment, the carbonyl group of the cleavable portion of the Linker L⁴ or L1 is directly attached to the bifunctional protein degrader, or with the carbonyl group being directly attached to the solubilizing portion of the linker. In another embodiment, the carbonyl group of the cleavable portion of the Linker L⁴ or L1 is directly attached to the solubilizing portion of the linker. In another embodiment, the cleavable portion of the Linker L⁴ or L1 is —C(O)CH₂N(H)—.

In another embodiment, the cleavable portion of the Linker L⁴ or L1 is *C(O)[C(Rₐ)₂]ₓN(R_{b})**, wherein each Rₐ is independently selected from the group consisting of H and C₁-C₃ alkyl, x is 1 or 2, R_{b} is selected from the group consisting of H and C₁-C₃ alkyl, * indicates the point of attachment to the Bifunctional Protein Degrader; and ** indicates the point of attachment to the solubilizing portion of the Linker L⁴ or L1.

In yet another embodiment, the cleavable portion of the Linker L⁴ or L1 is *—C(O)CH₂N(H)—** wherein * indicates the point of attachment to the bifunctional protein degrader and ** indicates the point of attachment to the solubilizing portion of the Linker L⁴ or the solubilizing domain.

In an embodiment, the bifunctional protein degrader, L⁴ or L1, and solubilizing domain, when present, have the structure of Formula (BFD-L1-SD-I):

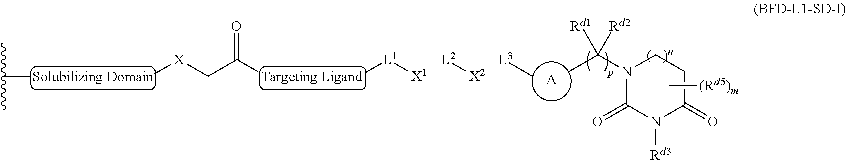

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-0, and *C(O)—C₁₋₉ heteroalkylene, wherein * denotes the point of attachment of L³ to X² in Formula (BFD-L1-SD-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2; X is O, S, C(R^{7a})(R^{7b}), N(R^{7c})C(O), C₁₋₆ alkyl, C₁₋₆ heteroalkyl, cycloalkyl, heterocyclyl, aryl, heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; and

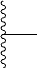

denotes the point of attachment to the fatty acid in Formula (I) or (I′).

In an embodiment, the bifunctional protein degrader, L⁴ or L1, and solubilizing domain, when present, have the structure of Formula (BFD-L1-SD-Ia):

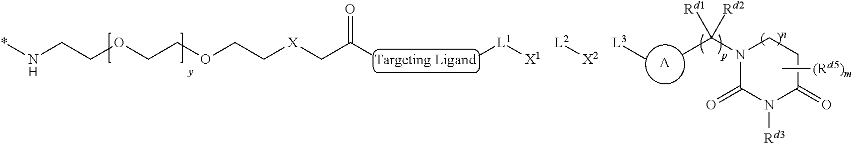

or a pharmaceutically acceptable salt, hydrate, solvate, prodrug, stereoisomer, or tautomer thereof, wherein L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand; X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen; L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-0, and *C(O)—C₁₋₉ heteroalkylene, wherein * denotes the point of attachment of L³ to X² in Formula (BFD-L1-SD-I); wherein no more than 2 of L¹, X¹, X², L², and L³ can simultaneously be a bond; ring A is a 6-membered aryl, or 5- or 6-membered heteroaryl, each of which is substituted with 0-4 occurrences of R^{d4}; R^{d1} and R^{d2} are each independently selected from the group consisting of H, C₁₋₆ alkyl, C₁₋₆ haloalkyl, C₁₋₆ heteroalkyl, and C₃₋₆ cycloalkyl; R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂; each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl; each R^{d5} is independently selected from the group consisting of H, C₁₋₆ alkyl, halogen, C₁₋₆ haloalkyl, C₁₋₆ heteroalkyl, and C₃₋₆ cycloalkyl; or two R^{d5} together with the carbon atoms to which they are attached form a cycloalkyl; or two R^{d5} attached to the same carbon atom form a C₃₋₄ spirocycloalkyl; R^{p} is H or C₁₋₆ alkyl; p is 0, 1, or 2; m is 1 or 2; and n is 1 or 2; X is O, S, C(R^{7a})(R^{7b}), N(R^{7c})C(O), C₁₋₆ alkyl, C₁₋₆ heteroalkyl, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is an integer between 0 and 35; and * indicates the point of attachment to the fatty acid.

### Fatty Acids

The present disclosure features fatty acid-bifunctional protein degrader conjugates further comprising a fatty acid component, e.g., which provides improved pharmacokinetic and/or pharmacodynamic and/or protein binding capabilities of the conjugate compared to a conjugate that does not comprise a fatty acid. In an embodiment, the conjugate of Formula (I) or (I′) comprises a fatty acid capable of binding to a protein (e.g., a soluble or membrane protein, e.g., albumin). In some embodiments, the fatty acid improves the plasma stability half-life, e.g., compared to a conjugate that does not comprise a fatty acid.

In an embodiment, the Fatty Acid and Solubilizing Domain have Formula SD-FA-I:

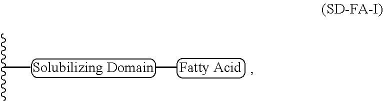

wherein the Solubilizing Domain comprises a heteroalkylene and is soluble in aqueous solution; the Fatty Acid comprises a fatty acid capable of binding to a protein; and

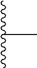

denotes the point of attachment to the bifunctional protein degrader via the linker (L1 or L⁴).

In an embodiment, the linker (L1), Solubilizing Domain, and Fatty Acid have Formula L1-SD-FA-I:

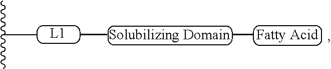

wherein the Solubilizing Domain comprises a heteroalkylene and is soluble in aqueous solution; the Fatty Acid comprises a fatty acid capable of binding to a protein; L1 comprises a cleavable linker; and

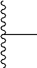

denotes the point of attachment to the bifunctional protein degrader. In an embodiment, Linker L⁴ comprises L1, which comprises a cleavable linker, and Solubilizing Domain.

In an embodiment, the linker, Solubilizing Domain, and Fatty Acid have Formula L1-SD-FA-II:

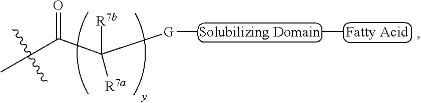

wherein the Solubilizing Domain comprises a heteroalkylene and is soluble in aqueous solution; the Fatty Acid comprises a fatty acid capable of binding to a protein; the variables G, R^{7a}, R^{7b}, and y are as defined herein; and

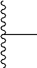

denotes the point of attachment to the bifunctional protein degrader. In an embodiment, Linker L⁴ comprises the cleavable linker and Solubilizing Domain.

In an embodiment, the linker, Fatty Acid and Solubilizing Domain have Formula L1-SD-FA-III(a) and L1-SD-FA-III(b):

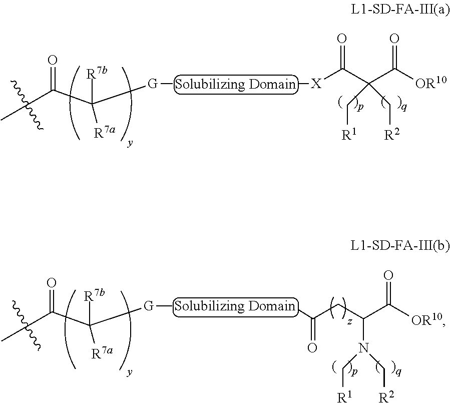

wherein the Solubilizing Domain comprises a heteroalkylene and is soluble in aqueous solution; the Fatty Acid comprises a fatty acid capable of binding to a protein; the variables G, R^{7a}, R^{7b}, R¹, R², R¹⁰, p, q, y, and z are as defined below; and

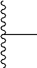

denotes the point of attachment to the bifunctional protein degrader. In an embodiment, Linker L⁴ comprises the cleavable linker and Solubilizing Domain.

In an embodiment, the fatty acid has a structure selected from the group consisting of Formula (FA-1), Formula (FA-2), and Formula (FA-3):

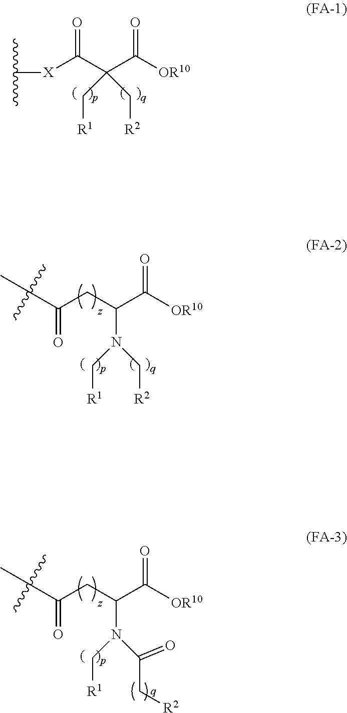

or a pharmaceutically acceptable salt, hydrate, solvate, prodrug, stereoisomer, or tautomer thereof, wherein X is O or N(R³); p and q are each an integer independently selected from 5 to 30; z is an integer selected from 0 to 5; R¹ and R² are each independently selected from CH₃, OR¹⁰, C(O)OR¹⁰, and P(O)(OR¹⁰)₂; R³ and R¹⁰ are each independently H or C₁₋₆ alkyl; and

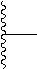

denotes the point of attachment to the solubilizing domain, when present, in Formula (I) or (I′). When the solubilizing domain is not present in Formula (I) or (I′), then

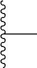

denotes the point of attachment to L1 or L⁴.

In an embodiment, the fatty acid has a structure of Formula (FA-1).

In an embodiment, the fatty acid of Formula (FA-1) has a structure selected from Formula (FA-1a) and (FA-1b):

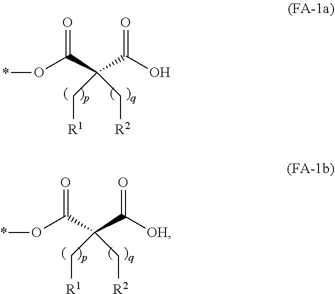

or a pharmaceutically acceptable salt, hydrate, solvate, prodrug, stereoisomer, or tautomer thereof, wherein p and q are each an integer independently selected from 5 to 30; R¹ and R² are each independently selected from CH₃, OR¹⁰, C(O)OR¹⁰, and P(O)(OR¹⁰)₂; R¹⁰ are each independently H or C₁₋₆ alkyl; and * denotes the point of attachment to the solubilizing domain, when present, in Formula (I) or (I′). When the solubilizing domain is not present in Formula (I) or (I′), then * denotes the point of attachment to L1 or L⁴.

In one embodiment, p and q are each an integer independently selected from 6 to 25, e.g., 6 to 20, e.g., 8 to 16, e.g., 8 to 14, and e.g., 10 to 14. In another embodiment, p and q are each independently 10 or each independently 14. In yet another embodiment, p and q are each independently 10.

In one embodiment, R¹ is CO₂H. In another embodiment, R² is CH₃ or CO₂H. In yet another embodiment, R² is CH₃. In another embodiment, R¹ is CO₂H and R² is CH₃.

In an embodiment, the fatty acid has a structure of Formula (FA-2). In an embodiment, the fatty acid of Formula (FA-2) has a structure selected from Formula (FA-2a) and (FA-2b):

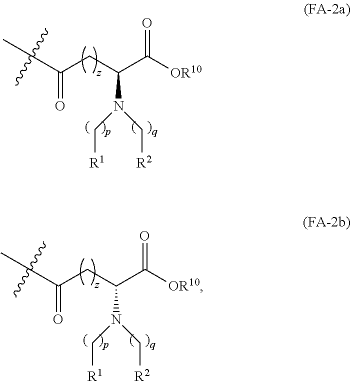

or a pharmaceutically acceptable salt, hydrate, solvate, prodrug, stereoisomer, or tautomer thereof, wherein p and q are each an integer independently selected from 5 to 30; z is an integer selected from 0 to 5; R¹ and R² are each independently selected from CH₃, OR¹⁰, C(O)OR¹⁰, and PP(O)(OR¹⁰)₂; R¹⁰ is H or C₁₋₆ alkyl; and

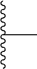

denotes the point of attachment to the solubilizing domain, when present, in Formula (I) or (I′). When the solubilizing domain is not present in Formula (I) or (I′), then

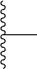

denotes the point of attachment to L1 or L⁴.

In an embodiment, the fatty acid has a structure of Formula (FA-3). In an embodiment, the fatty acid of Formula (FA-3) has a structure selected from Formula (FA-3a) and (FA-3b):

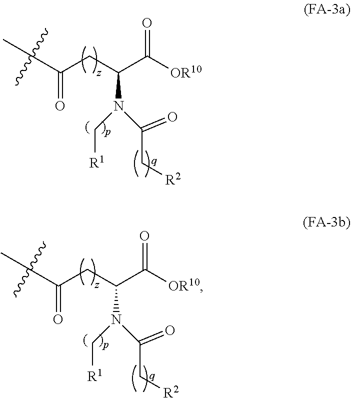

or a pharmaceutically acceptable salt, hydrate, solvate, prodrug, stereoisomer, or tautomer thereof, wherein p and q are each an integer independently selected from 5 to 30; z is an integer selected from 0 to 5; R¹ and R² are each independently selected from CH₃, OR¹⁰, C(O)OR¹⁰, and P(O)(OR¹⁰)₂; R¹⁰ is H or C₁₋₆ alkyl; and

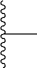

denotes the point of attachment to the solubilizing domain, when present, in Formula (I) or (I′).

In an embodiment, the conjugate of Formula (I), (I′) and subformula thereof has a plasma stability half-life of more than 10 hours, e.g., more than 20 hours, e.g., more than 30 hours. In an embodiment, the improvement of plasma stability compared to the non-conjugated bifunctional degrader compound without L1, the solubilizing domain, and the fatty acid is at least 2 fold, e.g., at least 5 fold, at least 10 fold, at least 20 fold, at least 30 fold, at least 40 fold, at least 50 fold, or at least 75 fold.

### Conjugates

In one aspect, the disclosure provides a conjugate of Formula (I):

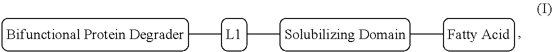

or a pharmaceutically acceptable salt, hydrate, solvate, prodrug, stereoisomer, or tautomer thereof, wherein
(i) a bifunctional protein degrader comprises a bifunctional compound capable of binding to each of a target protein and a ligase independently;
(ii) L1 comprises a cleavable linker;
(iii) optionally, a solubilizing domain comprising a heteroalkylene and is soluble in aqueous solution; and
(iv) a fatty acid comprises a fatty acid capable of binding to a protein.

In another aspect, provided herein is a conjugate of Formula (I′):

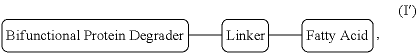

or a pharmaceutically acceptable salt, hydrate, solvate, prodrug, stereoisomer, or tautomer thereof, wherein
(i) a Bifunctional Protein Degrader is a Bruton's tyrosine kinase (BTK) Degrader capable of degrading BTK; and
(ii) a Linker is absent or L⁴, wherein L⁴ is a group that is cleavable to allow release of the Bifunctional Protein Degrader, and that covalently links the Bifunctional Protein Degrader to a Fatty Acid.

In some embodiments, the conjugate of Formula (I) has a Formula (I′a):

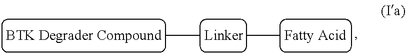

or a pharmaceutically acceptable salt, hydrate, solvate, prodrug, stereoisomer, or tautomer thereof.

In some embodiments, the conjugate of Formula (I) or (I′a), or a pharmaceutically acceptable salt, hydrate, solvate, prodrug, stereoisomer, or tautomer thereof, has a Linker L⁴, wherein L⁴ comprises L1 and, optionally, a solubilizing domain.

In an embodiment, the conjugate, or a pharmaceutically acceptable salt, hydrate, solvate, prodrug, stereoisomer, or tautomer thereof, comprises
i) a bifunctional protein degrader having a structure selected from the group consisting of Formula (BFD-BTK-I), (BFD-BTK-II), and (BFD-BTK-III):

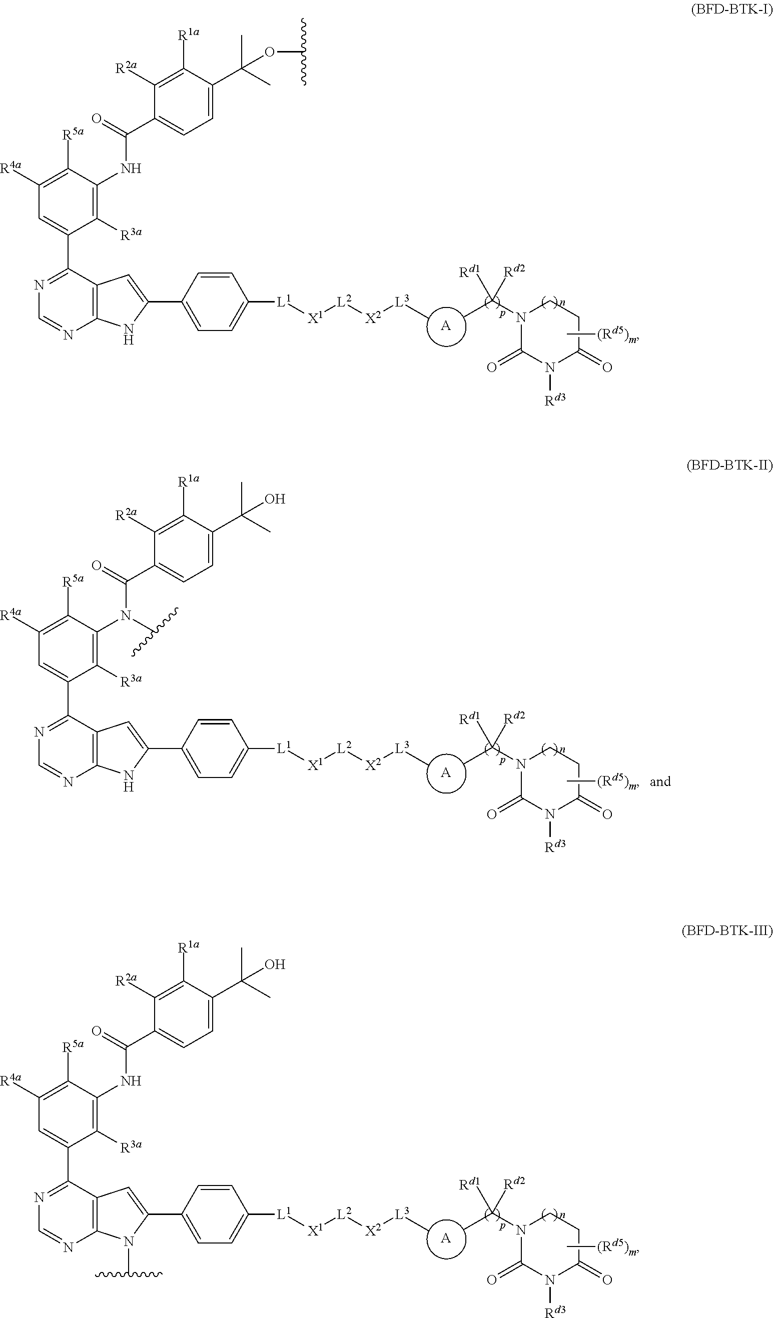

or a pharmaceutically acceptable salt, hydrate, solvate, prodrug, stereoisomer, or tautomer thereof, wherein
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to X¹;
X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen;
L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or
X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X²
wherein no more than 2 of L¹, X¹, X², L², and L³ can simultaneously be a bond;
ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl; or
two R^{d5} together with the carbon atoms to which they are attached form a cycloalkyl; or
two R^{d5} attached to the same carbon atom form a C₃₋₄ spirocycloalkyl;
R^{p} is H or C₁₋₆ alkyl;
p is 0, 1, or 2;
m is 1 or 2;
n is 1 or 2;
R^{1a} is H or halo;
R^{2a} is H or halo;
R^{3a} is C₁₋₆ alkyl;
R^{4a} is H or halo;
R^{5a} is H or halo; and

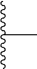

denotes the point of attachment to L1 in Formula (I) or the point of attachment to the Linker L⁴ in Formula (I) or Fatty Acid when the Linker is absent; and
ii) a Fatty Acid having a structure selected from the group consisting of Formula (FA-1), Formula (FA-2), and Formula (FA-3):

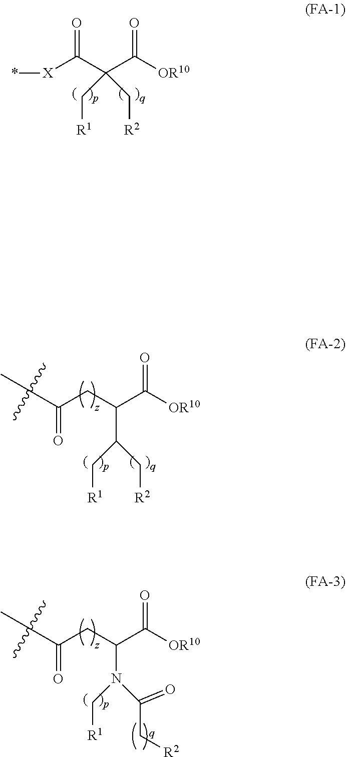

or a pharmaceutically acceptable salt, hydrate, solvate, prodrug, stereoisomer, or tautomer thereof, wherein
X is O or N(R³)
   p and q are each an integer independently selected from 5 to 30;
   z is an integer selected from 0 to 5;
   R¹ and R² are each independently selected from CH₃, OR¹⁰, C(O)OR¹⁰, and P(O)(OR¹⁰)₂;
   R³ and R¹⁰ are each independently H or C₁₋₆ alkyl; and

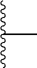

denotes the point of attachment to the solubilizing domain, when present, in Formula (I) or (I′).

In an embodiment, the conjugate, or a pharmaceutically acceptable salt, hydrate, solvate, prodrug, stereoisomer, or tautomer thereof, comprises
i) a bifunctional protein degrader having a structure selected from the group consisting of Formula (BFD-BTK-I-a), (BFD-BTK-II-a), (BFD-BTK-III-a), (BFD-BTK-I-b), (BFD-BTK-II-b), and (BFD-BTK-III-b):

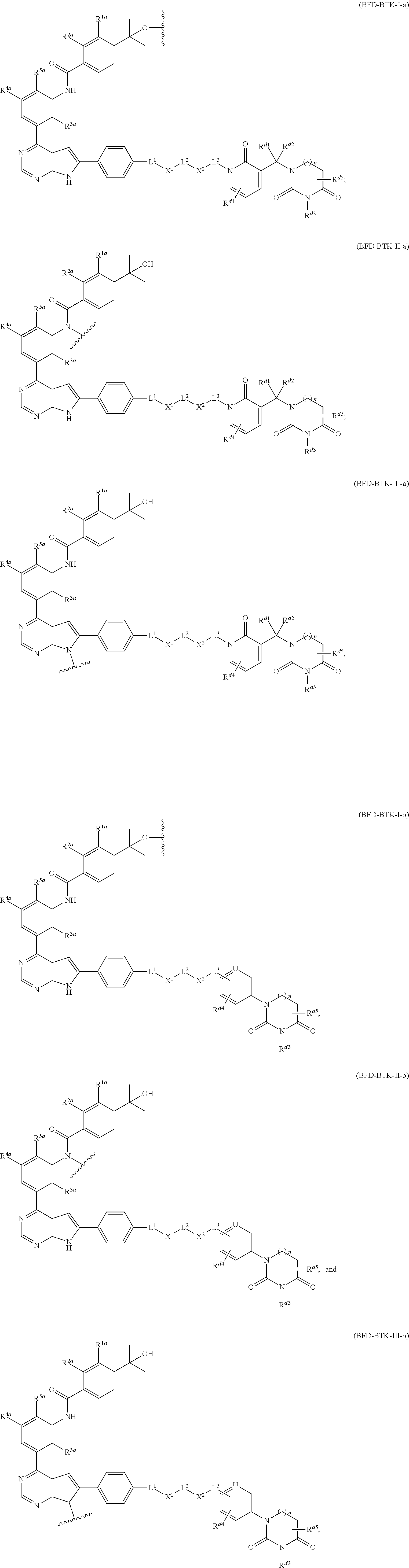

or a pharmaceutically acceptable salt, hydrate, solvate, prodrug, stereoisomer, or tautomer thereof, wherein
   L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to X¹;
   X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen;
   L² is selected from the group consisting of a bond, —O—, —NR′—, —C(O)—, C₁₋₆ alkylene, C₁₋₆ heteroalkylene; *C(O)NR′—C₁₋₆ alkylene, wherein * denotes the point of attachment of L² to X²; or
   X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen;
   L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X²;
   wherein no more than 2 of L¹, X¹, X², L², and L³ can simultaneously be a bond;
   R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
   R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
   R^{d4} is selected from the group consisting of H, oxo, hydroxyl, C₁₋₆ alkyl, halogen, C₁₋₆ haloalkyl, C₁₋₆ alkoxyl, and C₁₋₆ heteroalkyl;
   R^{d5} is selected from the group consisting of H, C₁₋₆ alkyl, halogen, C₁₋₆ haloalkyl, C₁₋₆ heteroalkyl, and C₃₋₆ cycloalkyl;
   U is —CR^{d6} or N;
   R^{d6} is selected from the group consisting of H, hydroxyl, C₁₋₆ alkyl, halogen, C₁₋₆ alkoxyl, C₁₋₆ haloalkyl, and C₁₋₆ heteroalkyl;
   R^{p} is H or C₁₋₆ alkyl;
   n is 1 or 2;
   R^{1a} is H or halo;
   R^{2a} is H or halo;
   R^{3a} is C₁₋₆ alkyl;
   R^{4a} is H or halo;
   R^{5a} is H or halo; and

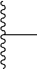

denotes the point of attachment to L1 in Formula (I) or the point of attachment to the Linker L⁴ in Formula (I′) or Fatty Acid when the Linker is absent; and
ii) a Fatty Acid having a structure selected from the group consisting of Formula (FA-1), Formula (FA-2), and Formula (FA-3):

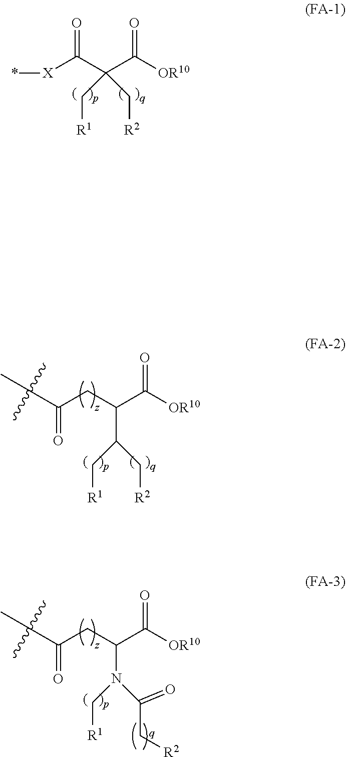

or a pharmaceutically acceptable salt, hydrate, solvate, prodrug, stereoisomer, or tautomer thereof, wherein
X is O or N(R³)
   p and q are each an integer independently selected from 5 to 30;
   z is an integer selected from 0 to 5;
   R¹ and R² are each independently selected from CH₃, OR¹⁰, C(O)OR¹⁰, and P(O)(OR¹⁰)₂;
   R³ and R¹⁰ are each independently H or C₁₋₆ alkyl; and

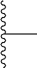

denotes the point of attachment to the solubilizing domain, when present, in Formula (I) or (I′).

In another embodiment, the conjugate of Formula (I) or (I′) has a Formula (I′b):

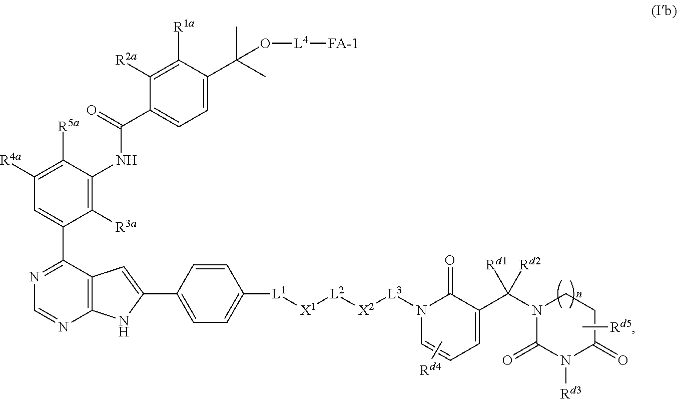

or a pharmaceutically acceptable salt, hydrate, solvate, prodrug, stereoisomer or tautomer thereof.

In another embodiment, the conjugate of Formula (I) or (I′) has a structure selected from the group consisting of Formula (II′b), (II′c), and (II′d):

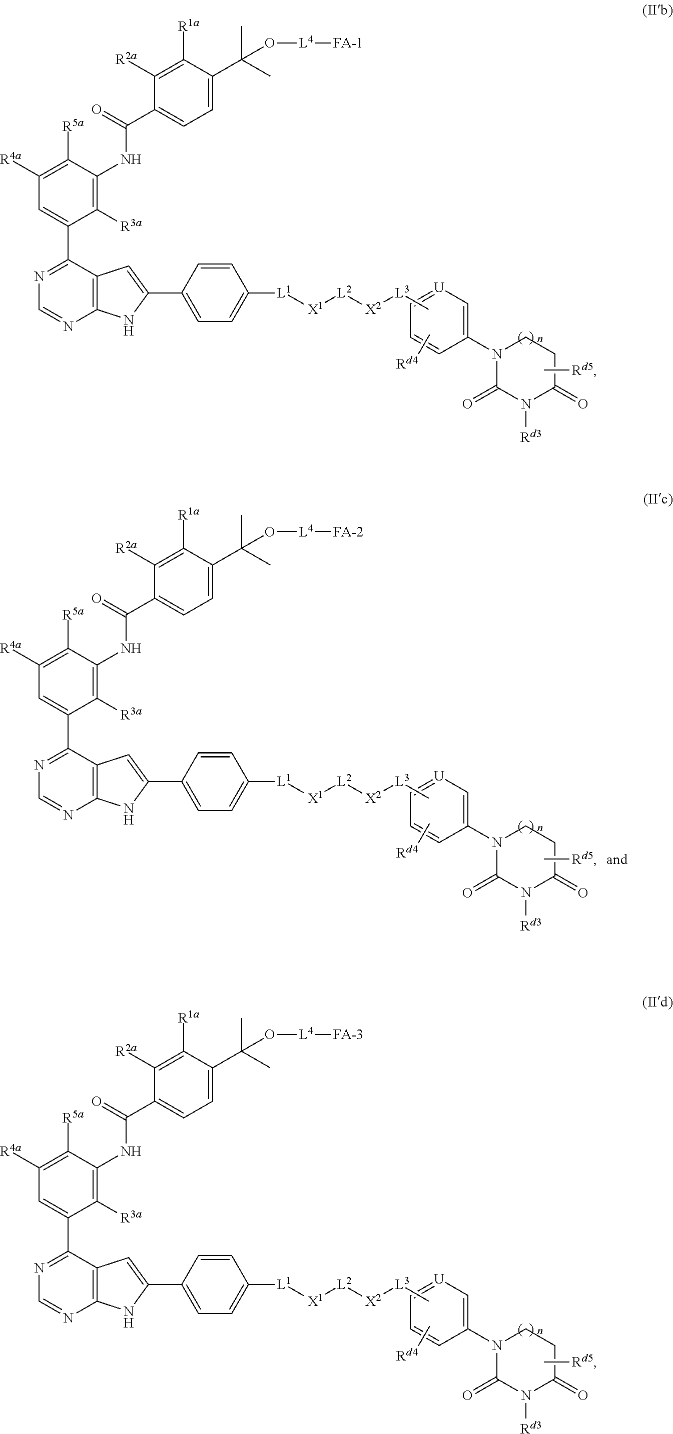

or a pharmaceutically acceptable salt, hydrate, solvate, prodrug, stereoisomer or tautomer thereof.

In an embodiment, the conjugate, or a pharmaceutically acceptable salt, hydrate, solvate, prodrug, stereoisomer, or tautomer thereof, wherein R^{2a} is fluoro. In an embodiment, R^{1a} is H. In an embodiment, R^{3a} is C₁-C₃ alkyl. In an embodiment, R^{3a} is methyl. In an embodiment, R^{4a} is fluoro. In an embodiment, R^{5a} is H.

In an embodiment, the conjugate, or a pharmaceutically acceptable salt, hydrate, solvate, prodrug, stereoisomer, or tautomer thereof, wherein L¹ is C₁-C₉ alkylene, e.g., C₁ alkylene.

In an embodiment, the conjugate, or a pharmaceutically acceptable salt, hydrate, solvate, prodrug, stereoisomer, or tautomer thereof, wherein X-L²-X²— is:

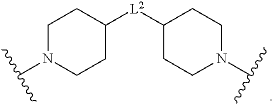

In an embodiment, the conjugate, or a pharmaceutically acceptable salt, hydrate, solvate, prodrug, stereoisomer, or tautomer thereof, wherein L² is —C(O)—, —O—, or C₁-C₆ alkylene. In an embodiment, L² is —O—.

In an embodiment, the conjugate, or a pharmaceutically acceptable salt, hydrate, solvate, prodrug, stereoisomer, or tautomer thereof, wherein L³ is selected from the group consisting of a bond, —O—, —C(O)—, —S(O)₂—, C₁-C₆ alkylene, C₂-C₆ alkynylene, and C₁-C₆ heteroalkylene. In an embodiment, L³ is C₁-C₆ alkylene. In an embodiment, L³ is C₂ alkylene.

In an embodiment, the conjugate, or a pharmaceutically acceptable salt, hydrate, solvate, prodrug, stereoisomer, or tautomer thereof, wherein R^{d4} is H. In an embodiment, R^{d1} is H. In an embodiment, R^{d2} is H. In an embodiment, R^{d1} and R^{d2} are both H. In an embodiment, n is 1. In an embodiment, R^{d3} is H. In an embodiment, R^{d5} is H or C₁-C₃ alkyl. In an embodiment, R^{d8} is H.

In another embodiment, the conjugate of Formula (I) or (I′) has a Formula (I′c):

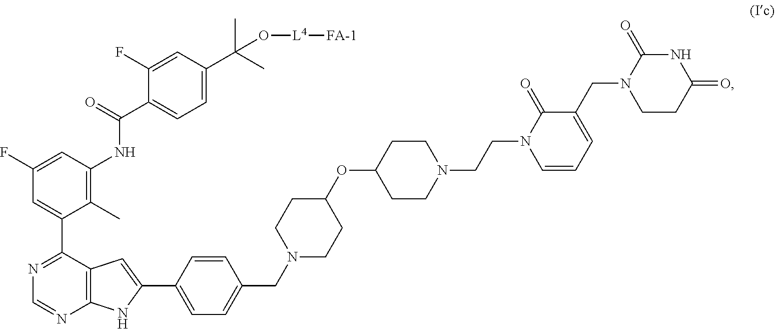

or a pharmaceutically acceptable salt, hydrate, solvate, prodrug, stereoisomer or tautomer thereof.

In another embodiment, the conjugate of Formula (I) or (I′) has a structure selected from the group consisting of Formula (II′e), (II′f), and (II′g):

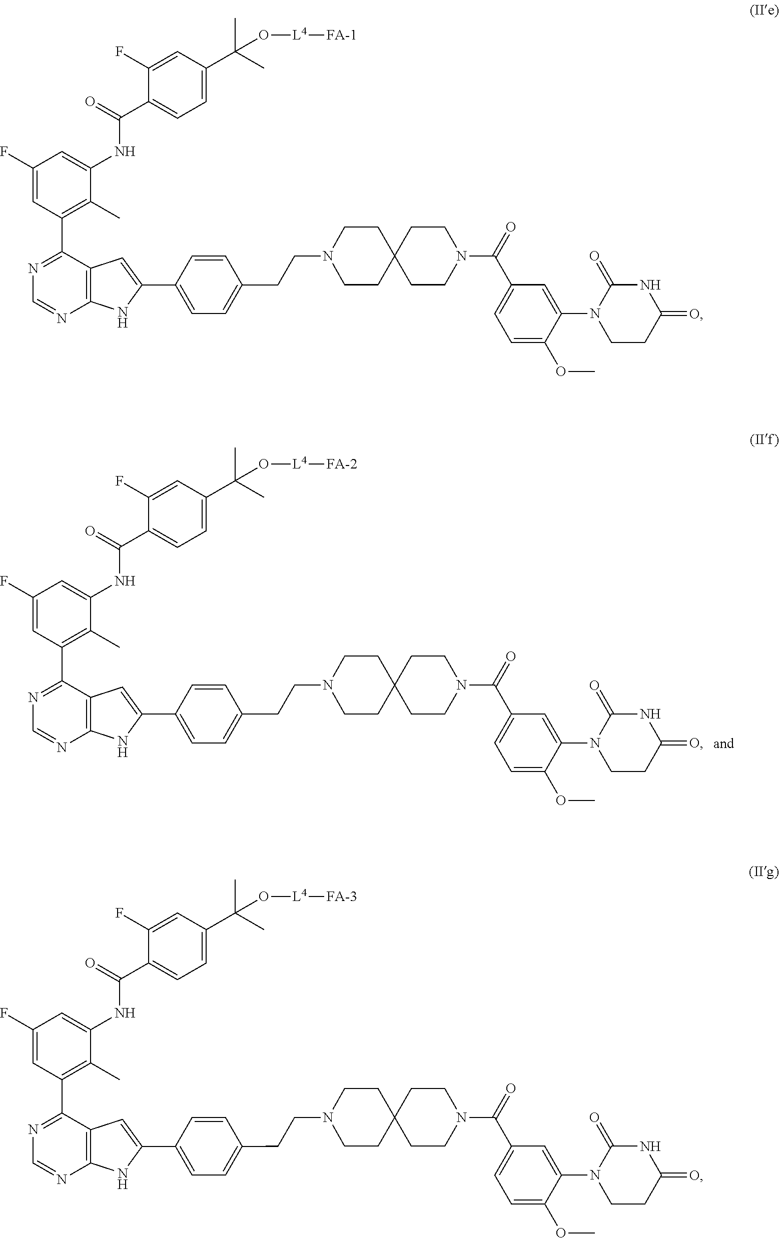

or a pharmaceutically acceptable salt, hydrate, solvate, prodrug, stereoisomer or tautomer thereof.

In another embodiment, the conjugate of Formula (I) or (I′) has a Formula (II′e):

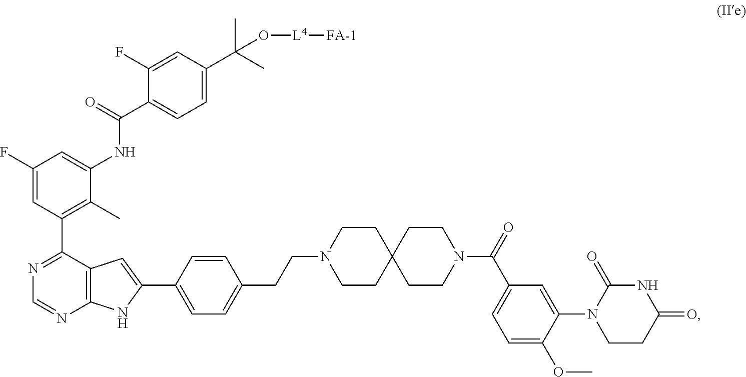

or a pharmaceutically acceptable salt, hydrate, solvate, prodrug, stereoisomer or tautomer thereof.

In an embodiment, the conjugate of Formula (I) or (I′) has a Formula (II′g):

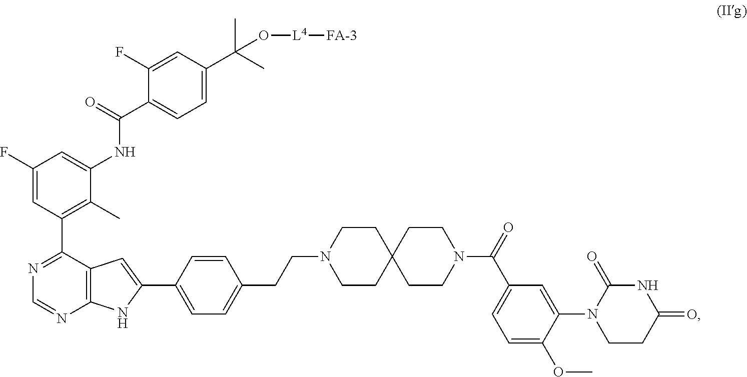

or a pharmaceutically acceptable salt, hydrate, solvate, prodrug, stereoisomer or tautomer thereof.

In an embodiment, the conjugate, or a pharmaceutically acceptable salt, hydrate, solvate, prodrug, stereoisomer, or tautomer thereof, wherein the Fatty Acid has a structure of Formula (FA-1):

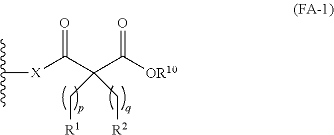

wherein X is O or N(R³); p and q are each an integer independently selected from 5 to 30; R¹ and R² are each independently selected from CH₃, OR¹⁰, C(O)OR¹⁰, and P(O)(OR¹⁰)₂; R³ and R¹⁰ are each independently H or C₁₋₆ alkyl; and

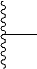

denotes the point of attachment to the solubilizing domain, when present, in Formula (I) or (I′).

In an embodiment, the fatty acid of Formula (FA-1) has a structure selected from Formula (FA-1a) and (FA-1b):

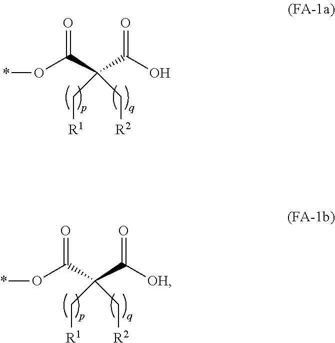

or a pharmaceutically acceptable salt, hydrate, solvate, prodrug, stereoisomer, or tautomer thereof.

In an embodiment, the conjugate, or a pharmaceutically acceptable salt, hydrate, solvate, prodrug, stereoisomer, or tautomer thereof, wherein the Fatty Acid has a structure of Formula (FA-2):

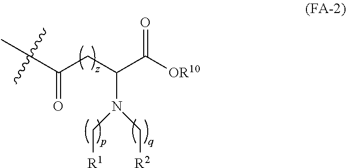

wherein p and q are each an integer independently selected from 5 to 30; z is an integer selected from 0 to 5; R¹ and R² are each independently selected from CH₃, OR¹⁰, C(O)OR¹⁰, and P(O)(OR¹⁰)₂; R¹⁰ is H or C₁₋₆ alkyl; and

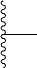

denotes the point of attachment to the solubilizing domain, when present, in Formula (I) or (I′). When the solubilizing domain is not present in Formula (I) or (I′), then

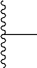

denotes the point of attachment to L1 or L⁴.

In an embodiment, the fatty acid of Formula (FA-2) has a structure selected from Formula (FA-2a) and (FA-2b):

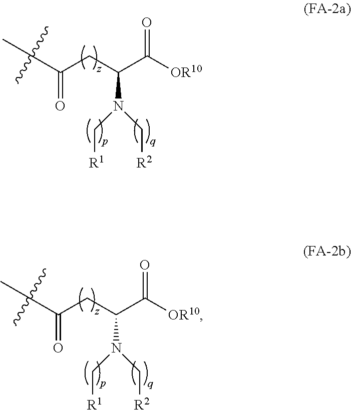

or a pharmaceutically acceptable salt, hydrate, solvate, prodrug, stereoisomer, or tautomer thereof.

In an embodiment, the conjugate, or a pharmaceutically acceptable salt, hydrate, solvate, prodrug, stereoisomer, or tautomer thereof, wherein the Fatty Acid has a structure of Formula (FA-3):

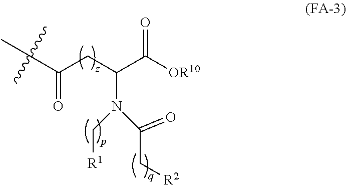

wherein p and q are each an integer independently selected from 5 to 30; z is an integer selected from 0 to 5; R¹ and R² are each independently selected from CH₃, OR¹⁰, C(O)OR¹⁰, and P(O)(OR¹⁰)₂; R¹⁰ is H or C₁₋₆ alkyl; and

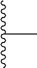

denotes the point of attachment to the solubilizing domain, when present, in Formula (I) or (I′). When the solubilizing domain is not present in Formula (I) or (I), then

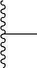

denotes the point of attachment to L1 or L⁴.

In an embodiment, the fatty acid of Formula (FA-3) has a structure selected from Formula (FA-3a) and (FA-3b):

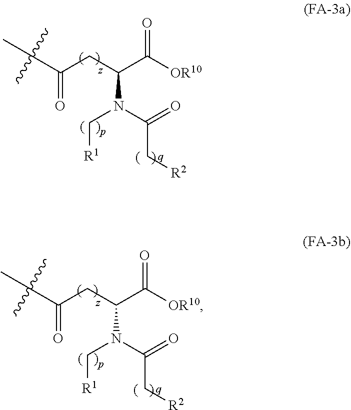

or a pharmaceutically acceptable salt, hydrate, solvate, prodrug, stereoisomer, or tautomer thereof.

In an embodiment, the conjugate, or a pharmaceutically acceptable salt, hydrate, solvate, prodrug, stereoisomer, or tautomer thereof, p and q are each an integer independently selected from 6 to 25, e.g., 6 to 20, e.g., 8 to 16, e.g., 8 to 14, or e.g., 10 to 14. In an embodiment, p and q are each independently 10 or each independently 14. In an embodiment, p and q are each independently 10.

In an embodiment, the conjugate, or a pharmaceutically acceptable salt, hydrate, solvate, prodrug, stereoisomer, or tautomer thereof, R¹ is CO₂H. In an embodiment, R² is CH₃ or CO₂H. In an embodiment, R² is CH₃. In an embodiment, R¹ is CO₂H and R² is CH₃.

In an embodiment, the conjugate, or a pharmaceutically acceptable salt, hydrate, solvate, prodrug, stereoisomer, or tautomer thereof, wherein a cleavable portion of the Linker L⁴ or L1 is directly attached to the Bifunctional Protein Degrader, e.g., via a terminal oxygen of the Bifunctional Protein Degrader, and a solubilizing portion of the Linker L⁴ or a solubilizing domain is directly attached to the Fatty Acid.

In an embodiment, the conjugate, or a pharmaceutically acceptable salt, hydrate, solvate, prodrug, stereoisomer, or tautomer thereof, wherein the solubilizing portion of the Linker L⁴ or a solubilizing domain comprises C₅-C₁₀₀ alkylene, C₅-C₁₀₀ alkenylene, C₅-C₁₀₀ heteroalkylene, C₅-C₂₀₀ haloalkylene or polyethylene glycol, or one or more natural or unnatural amino acids, or a combination thereof. In an embodiment, the solubilizing portion of the Linker L⁴ or the solubilizing domain comprises a moiety having one of the following formulae:

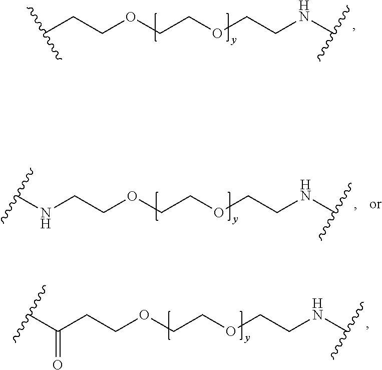

wherein y is 0 to 35.

In an embodiment, the solubilizing portion of the Linker L⁴ or the solubilizing domain comprises a moiety having one of the following formulae:

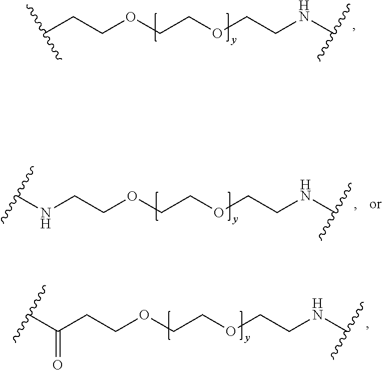

wherein y is 0 to 35, * indicates the point of attachment to the Fatty Acid, and ** indicates the point of attachment to the cleavable portion of the Linker L⁴ or L1.

In an embodiment, y is 1 to 35, e.g. 5 to 30, e.g., 6 to 25, e.g. 6 to 20, e.g., 7 to 15, e.g., 9 to 13, or e.g., 11.

In an embodiment, the conjugate, or a pharmaceutically acceptable salt, hydrate, solvate, prodrug, stereoisomer, or tautomer thereof, wherein the solubilizing portion of the linker L⁴ or the solubilizing domain is a moiety having the following formula:

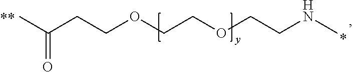

wherein * indicates the point of attachment to the Fatty Acid, ** indicates the point of attachment to the cleavable portion of the linker, and y is 11.

In an embodiment, the conjugate, or a pharmaceutically acceptable salt, hydrate, solvate, prodrug, stereoisomer, or tautomer thereof, wherein the cleavable portion of the Linker L⁴ or L1 is attached to the terminal oxygen atom of the Bifunctional Protein Degrader through an ester linkage, and wherein the solubilizing portion of the Linker L⁴ or the solubilizing domain is attached to the Fatty Acid through an amide linkage.

In an embodiment, the cleavable portion of the Linker L⁴ or L1 comprises a natural or unnatural amino acid which is attached to the terminal oxygen atom of the Bifunctional Protein Degrader through an ester linkage formed between the terminal oxygen and the carboxylic acid of the amino acid, and wherein the solubilizing portion of the Linker L⁴ or the solubilizing domain is attached to the Fatty Acid through an amide linkage. In an embodiment, the cleavable portion of the Linker L⁴ or L1 comprises a natural amino acid which is attached to the terminal oxygen atom of the Bifunctional Protein Degrader through an ester linkage formed between the terminal oxygen and the carboxylic acid of the amino acid and the solubilizing portion of the Linker L⁴ or the solubilizing domain is attached to the Fatty Acid through an amide linkage.

In an embodiment, the conjugate, or a pharmaceutically acceptable salt, hydrate, solvate, prodrug, stereoisomer, or tautomer thereof, wherein the cleavable portion of the Linker L⁴ or L1 is *C(O)[C(Rₐ)₂]ₓN(R_{b})**, wherein:
each Rₐ is independently selected from the group consisting of H and C₁-C₃ alkyl,
x is 1 or 2,
R_{b} is selected from the group consisting of H and C₁-C₃ alkyl,
* indicates the point of attachment to the Bifunctional Protein Degrader; and
** indicates the point of attachment to the solubilizing portion of the Linker L⁴ or the solubilizing domain.

In an embodiment, the cleavable portion of the Linker L⁴ or L1 is —C(O)CH₂N(H)—. In an embodiment, the cleavable portion of the Linker L⁴ or L1 is *—C(O)CH₂N(H)—**, wherein * indicates the point of attachment to the BTK Degrader Compound and ** indicates the point of attachment to the solubilizing portion of the Linker L⁴ or the solubilizing domain.

In some embodiments, the fatty acid-bifunctional protein degrader conjugate (e.g., of Formula (I) or (I′)), or a pharmaceutically acceptable salt, hydrate, solvate, prodrug, stereoisomer, or tautomer thereof, is selected from the group consisting of

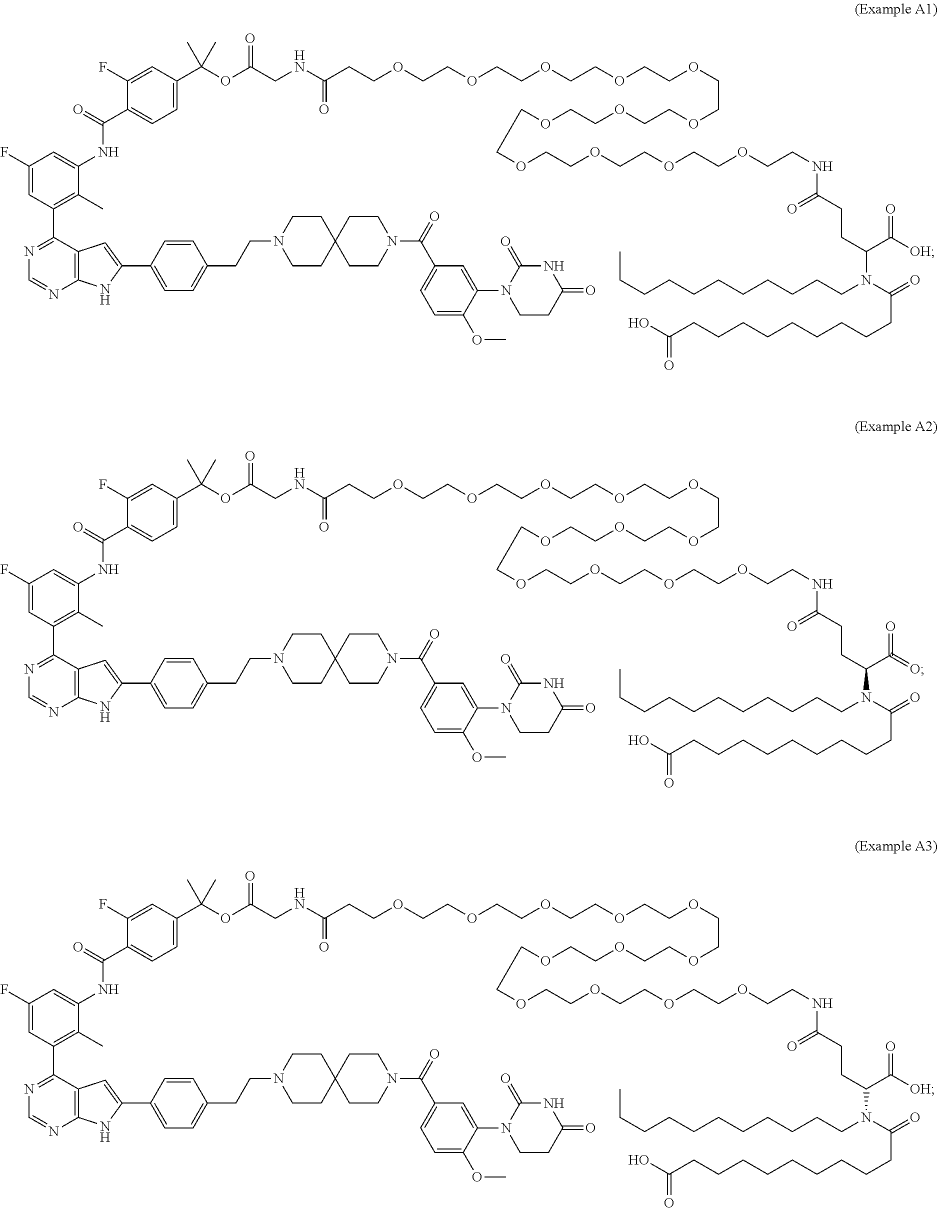

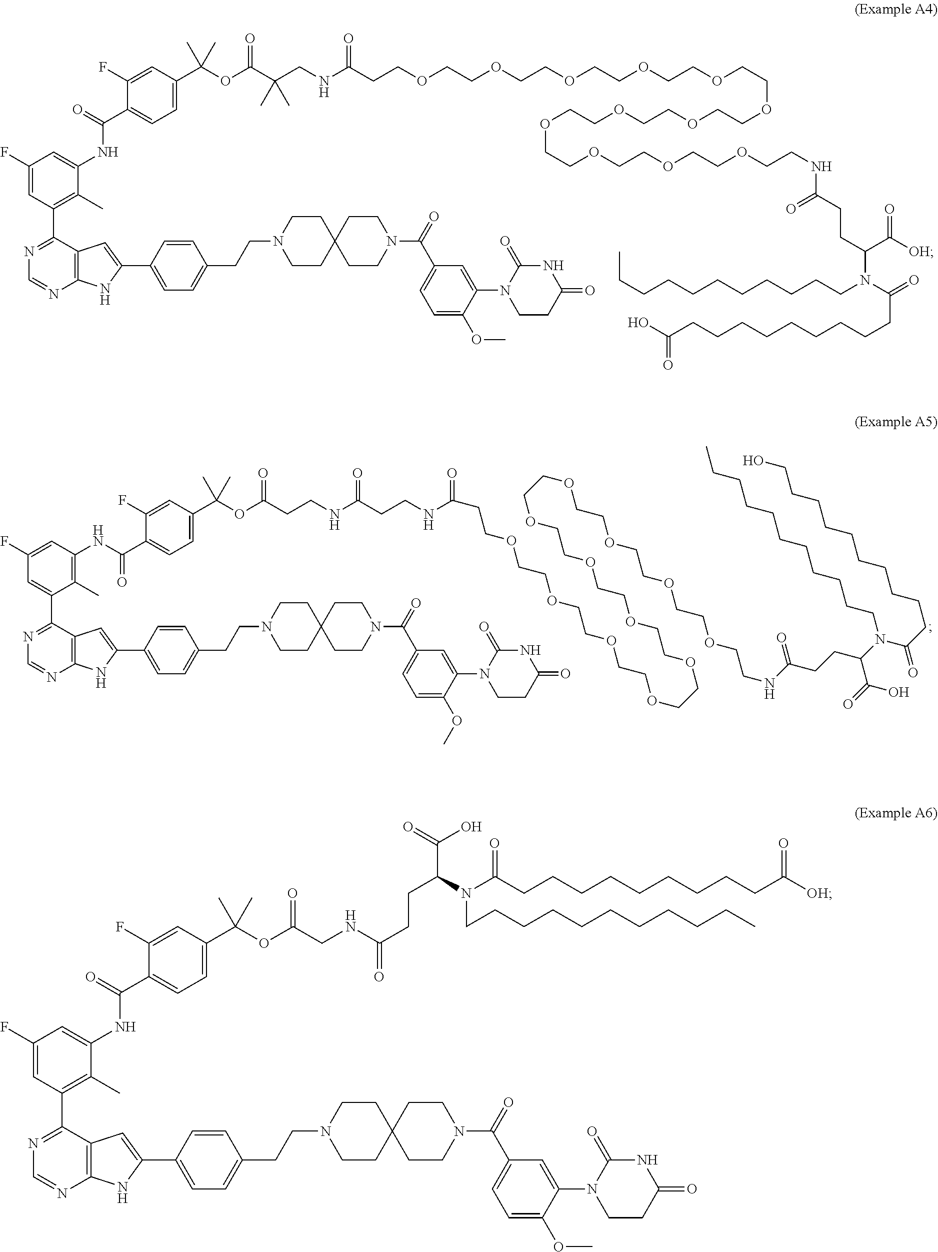

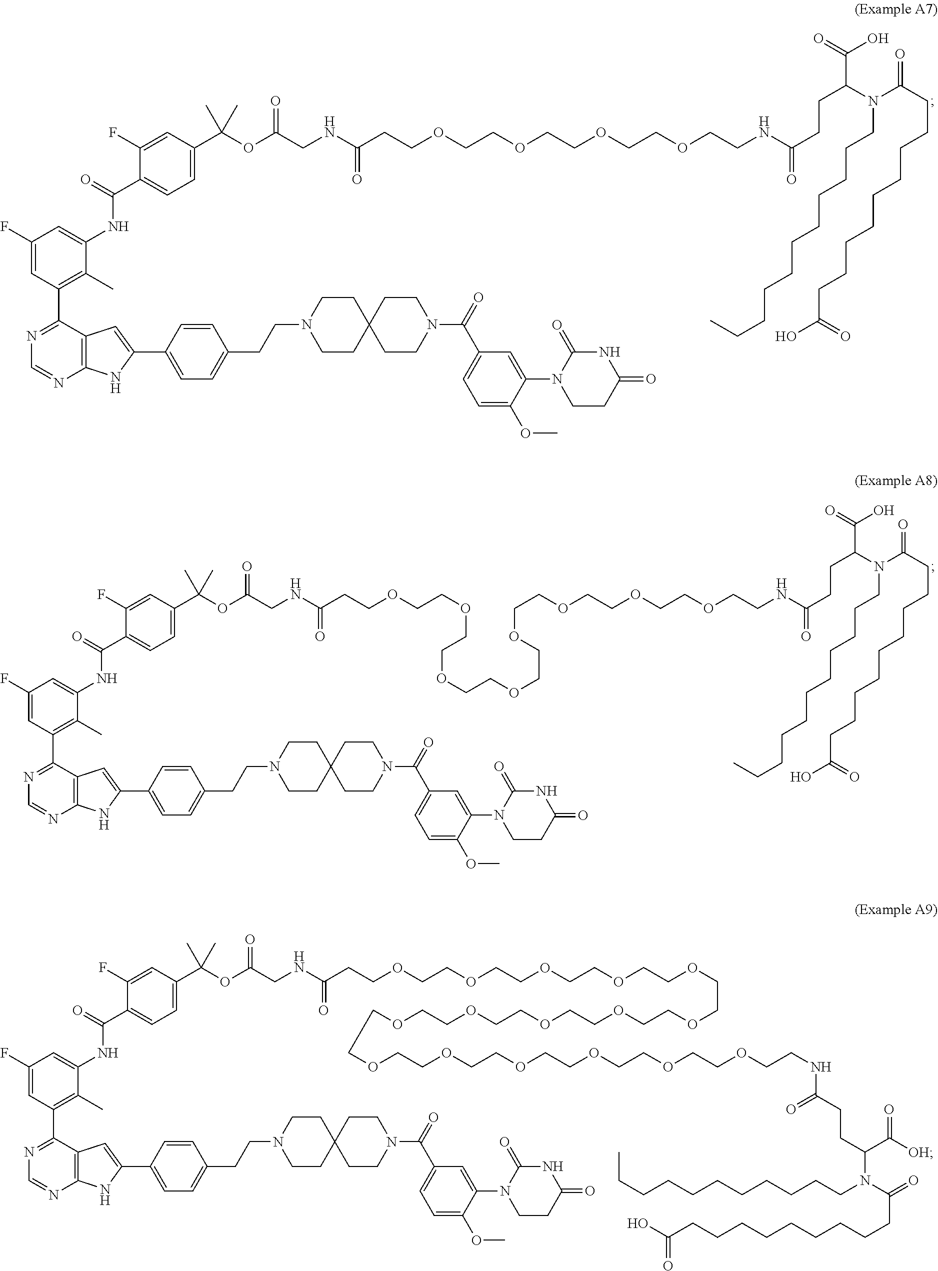

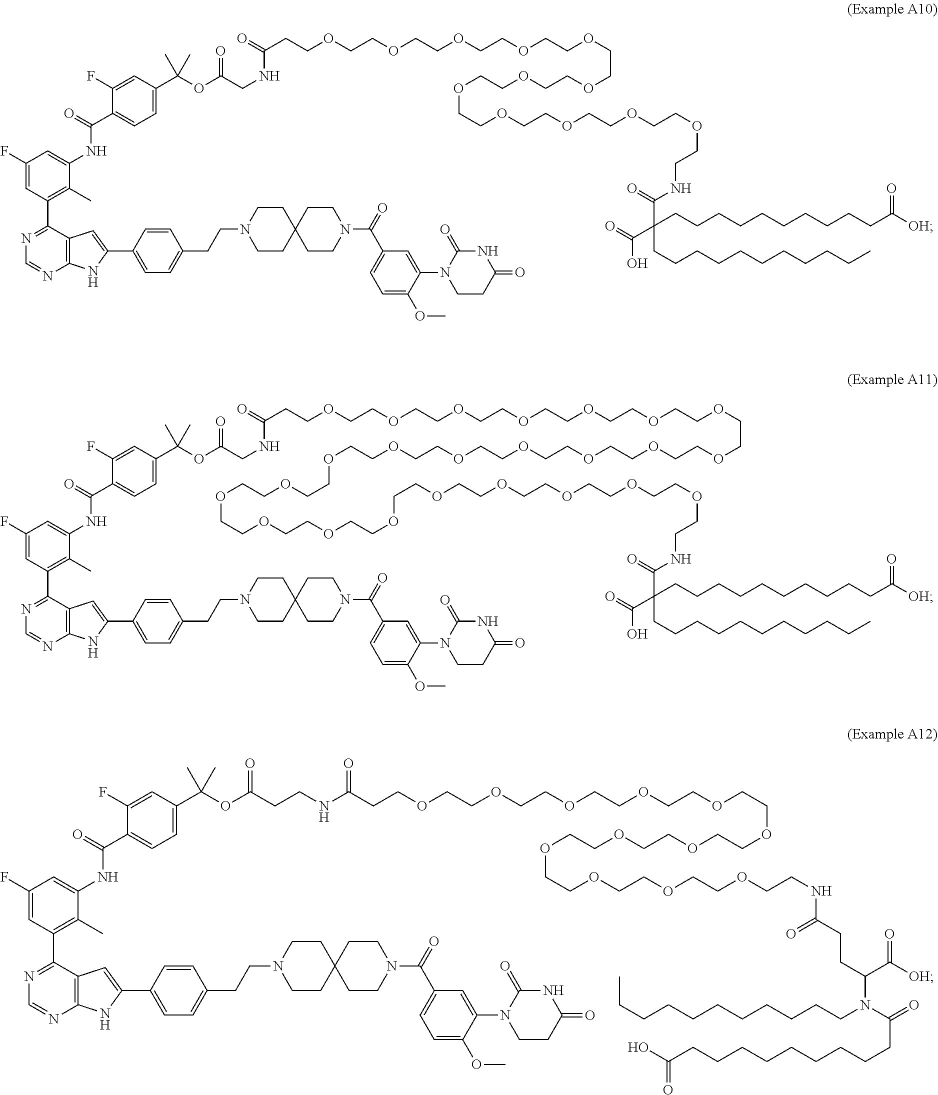

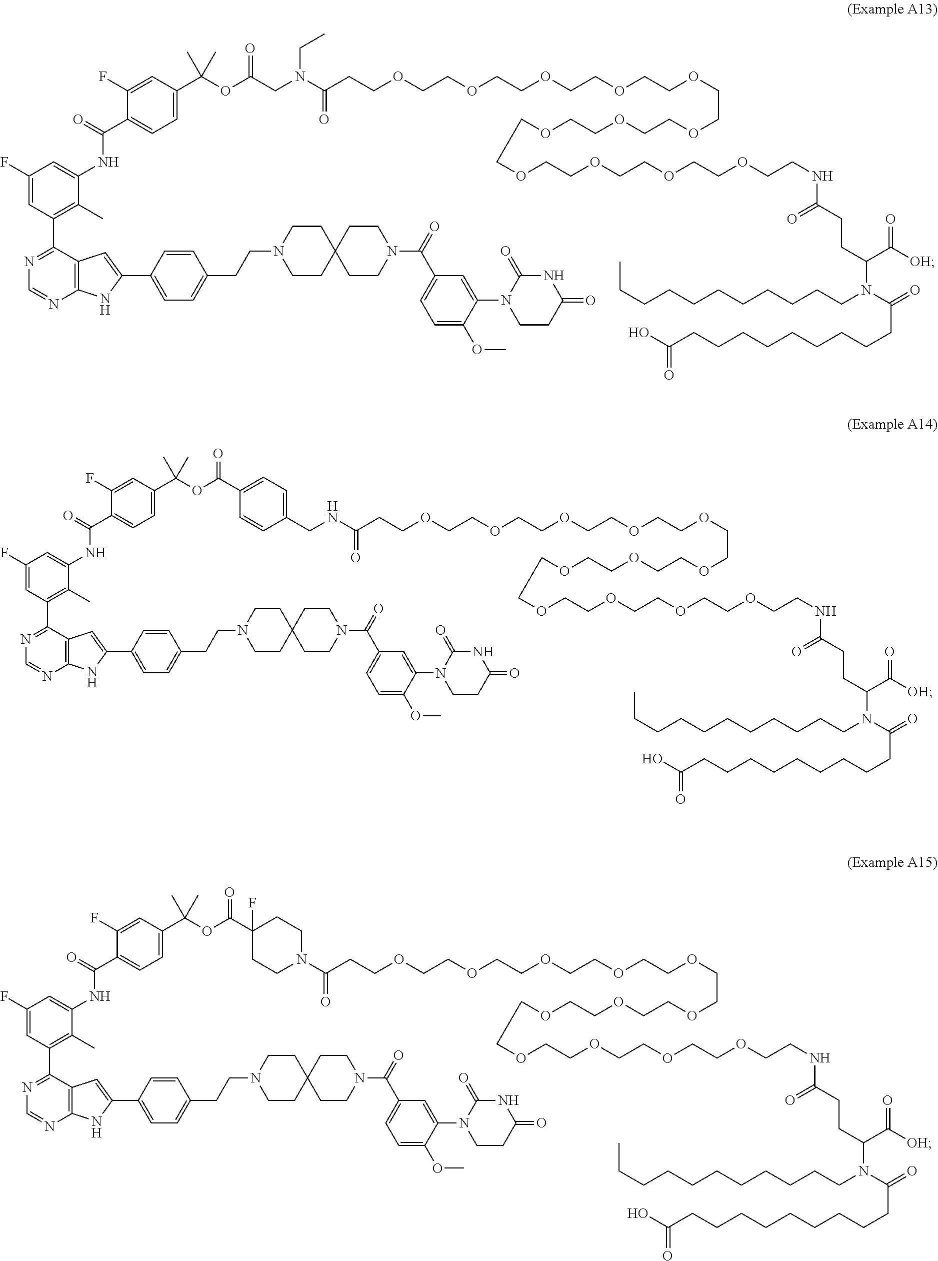

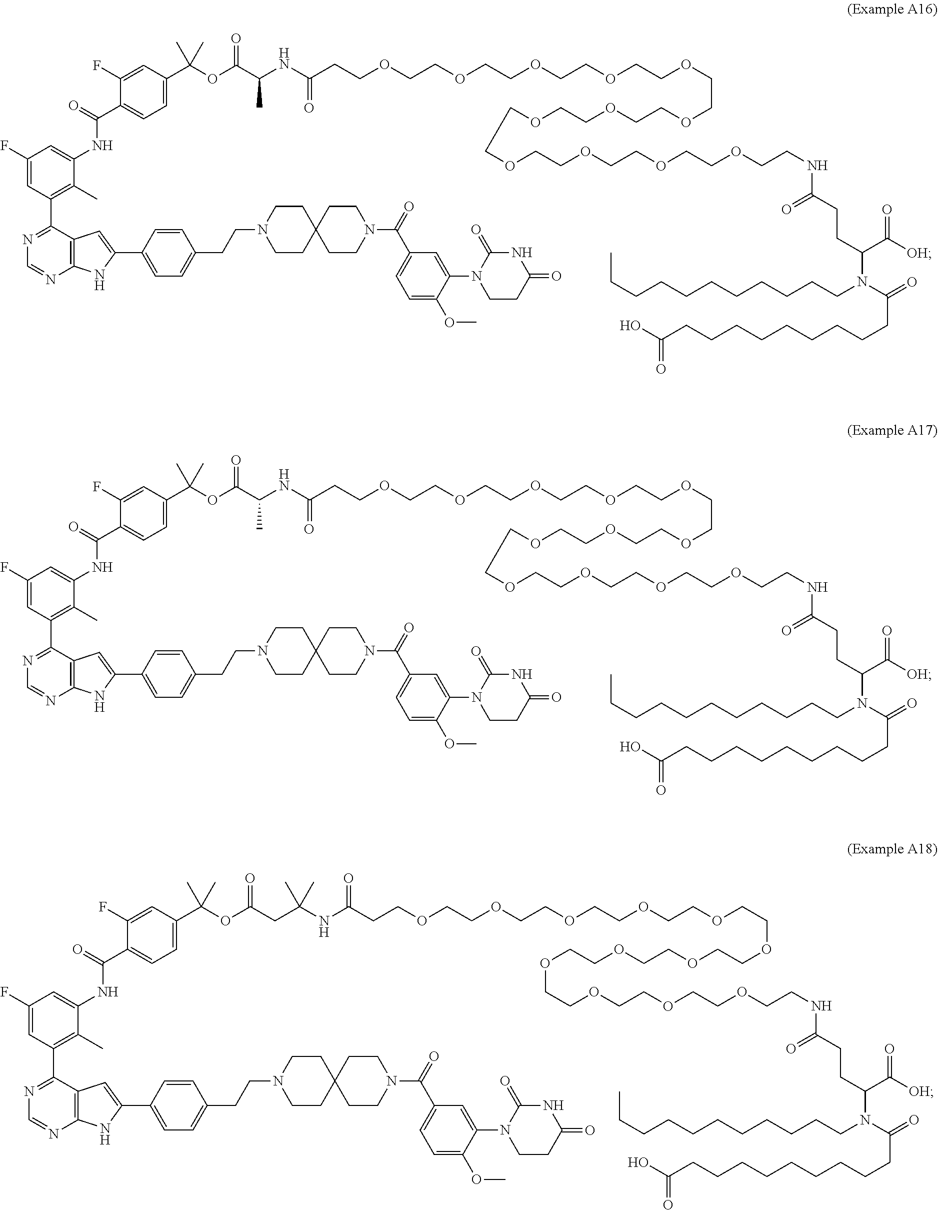

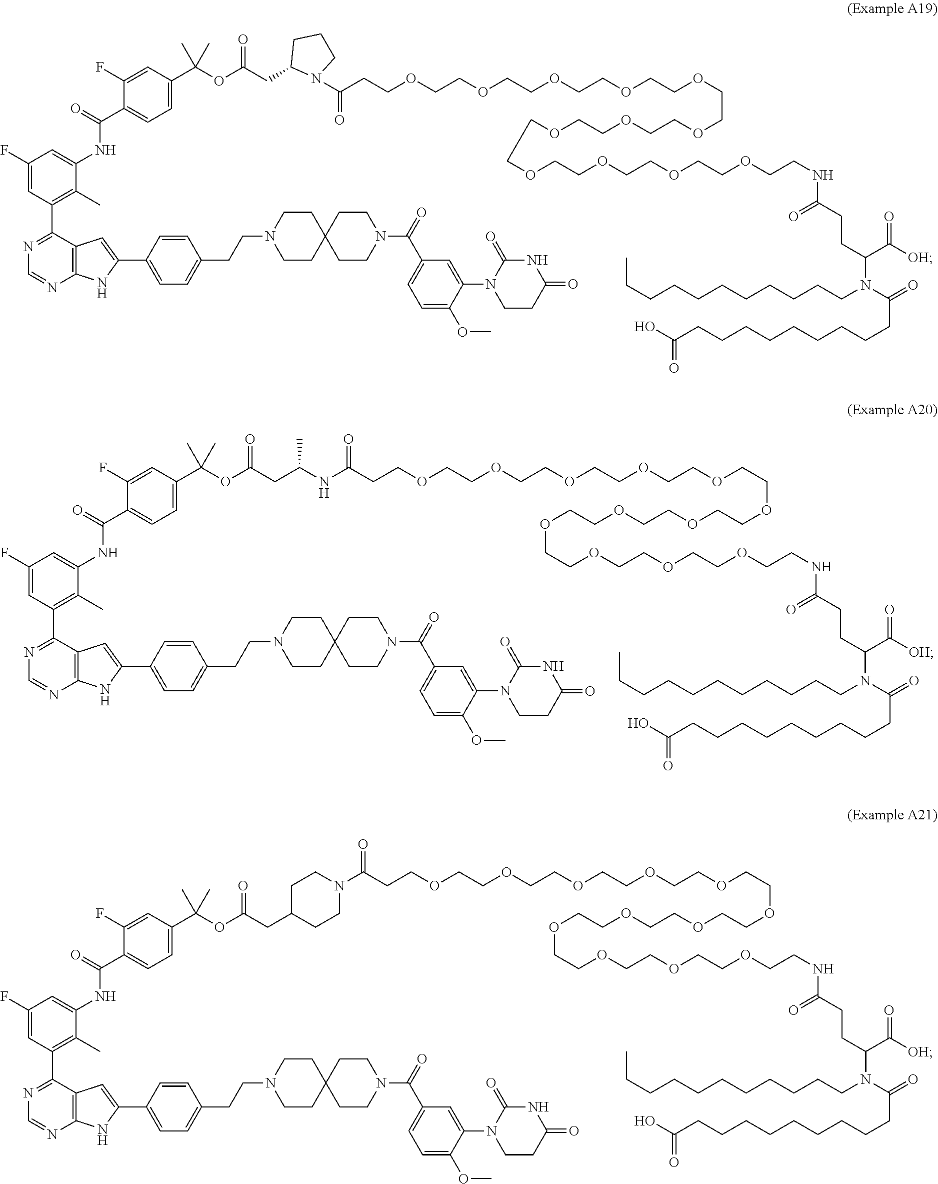

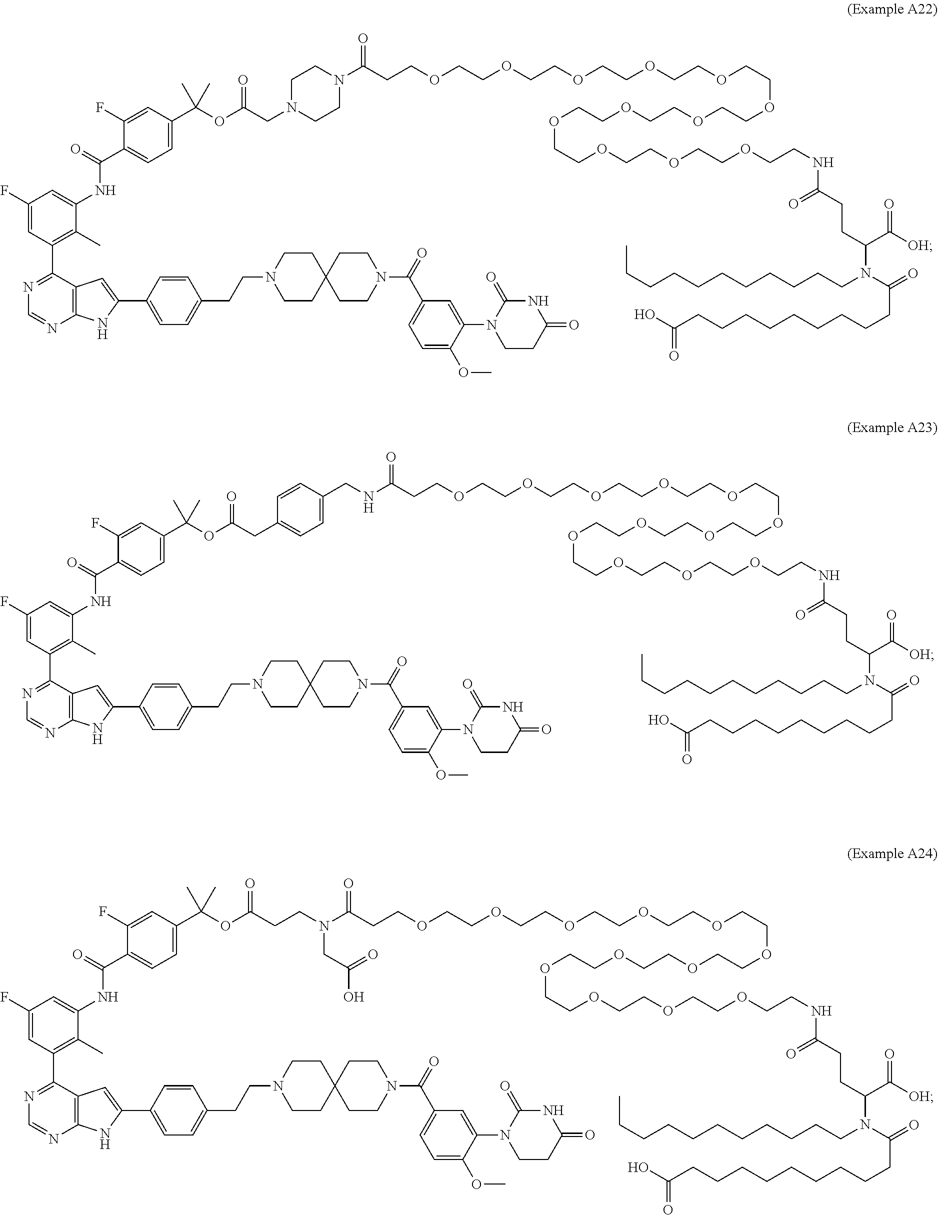

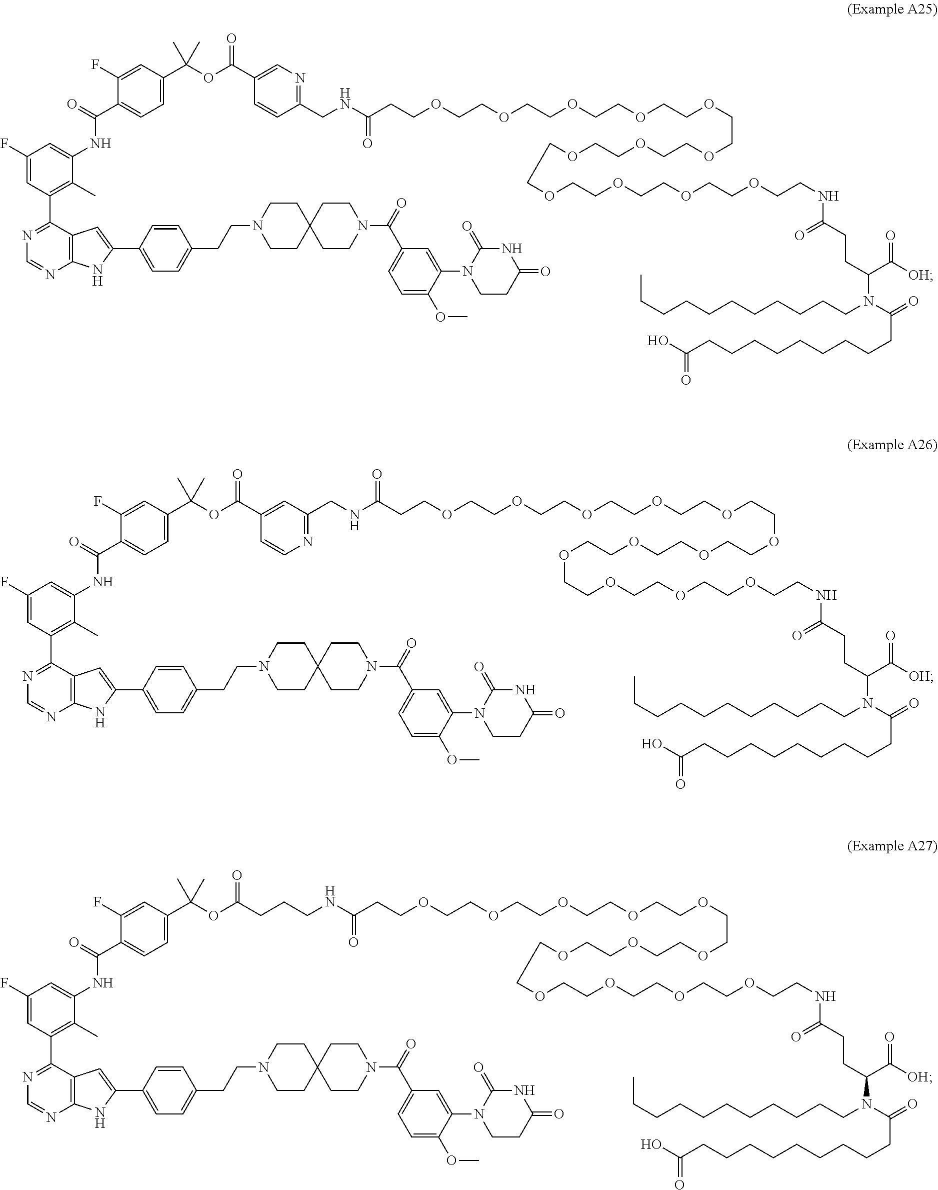

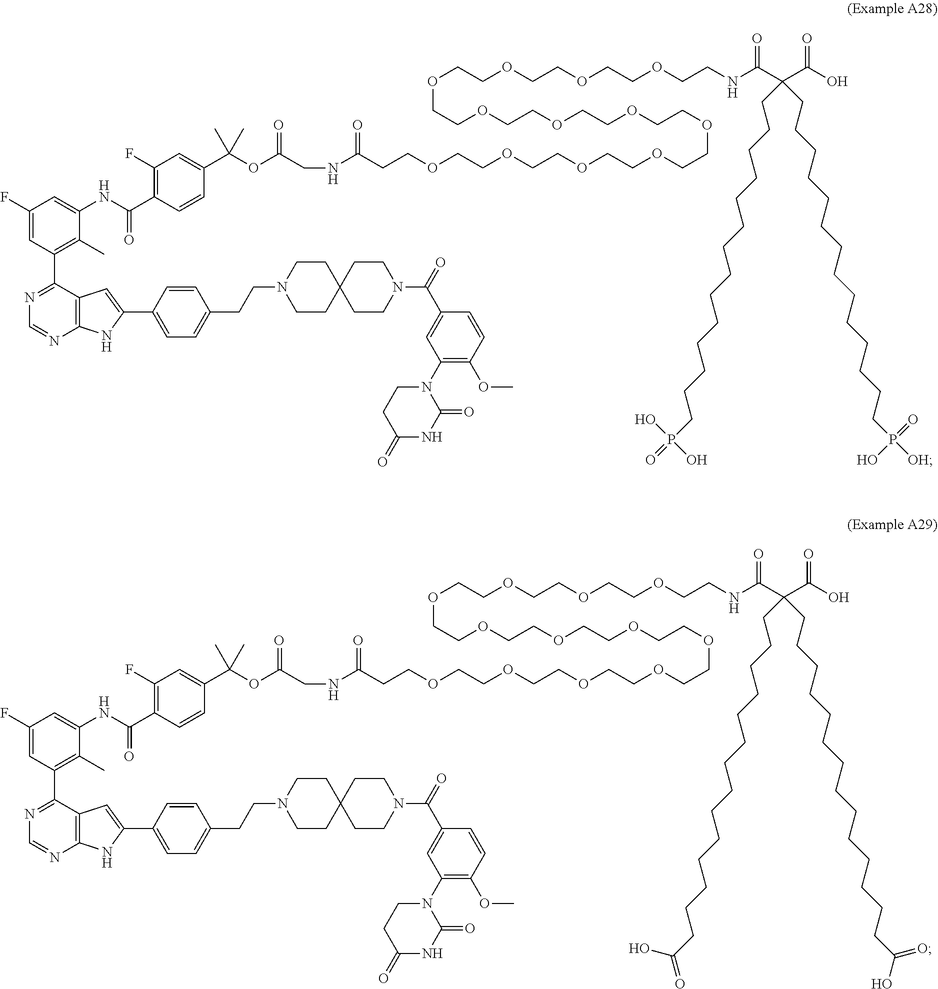 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCNC(=O)C(CCCCCCCCCCCCCCP(=O)(O)O)(CCCCCCCCCCCCCCP(=O)(O)O)C(=O)O)cc8F)c7C)ncnc6[nH]5)cc4)CC3)CC2)cc1N1CCC(=O)NC1=O ; COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCNC(=O)C(CCCCCCCCCCCCCCC(=O)O)(CCCCCCCCCCCCCCC(=O)O)C(=O)O)cc8F)c7C)ncnc6[nH]5)cc4)CC3)CC2)cc1N1CCC(=O)NC1=O]

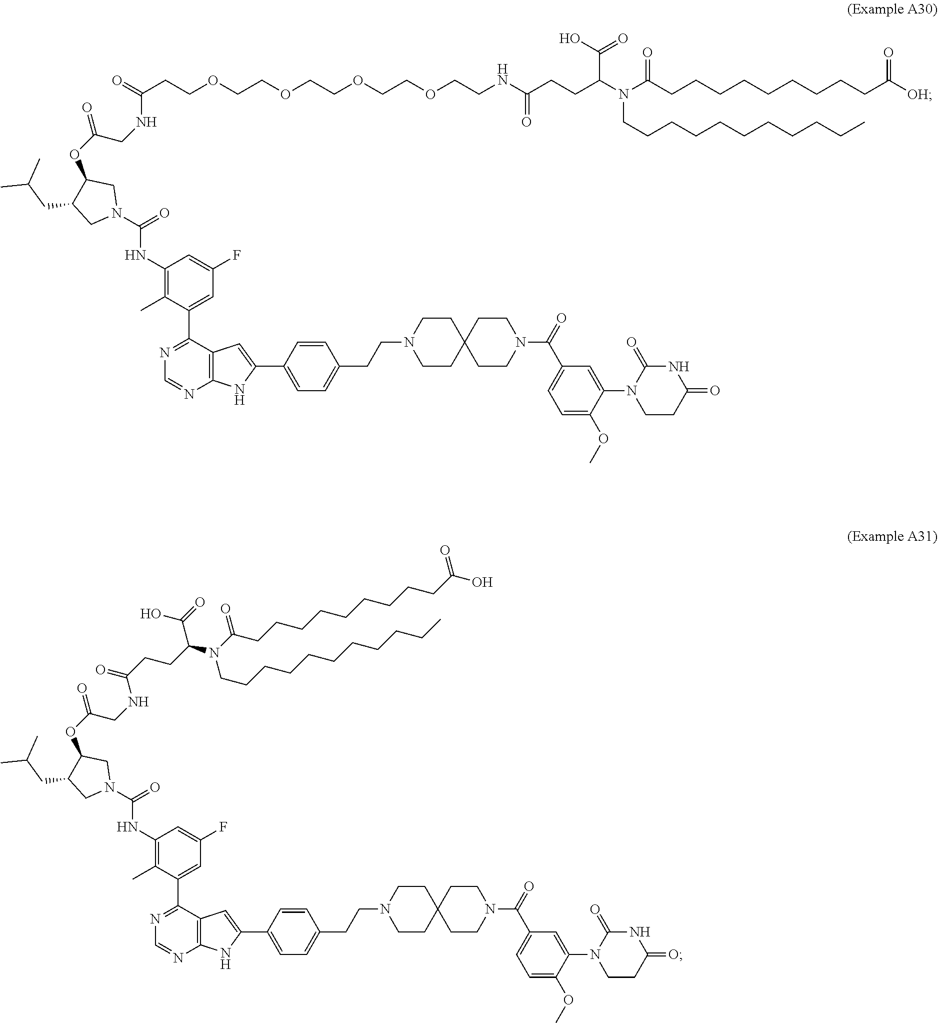 [SMILES: CCCCCCCCCCCN(C(=O)CCCCCCCCCC(=O)O)C(CCC(=O)NCCOCCOCCOCCOCCC(=O)NCC(=O)O[C@H]1CN(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)C[C@@H]1CC(C)C)C(=O)O ; CCCCCCCCCCCN(C(=O)CCCCCCCCCC(=O)O)[C@@H](CCC(=O)NCC(=O)O[C@H]1CN(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)C[C@@H]1CC(C)C)C(=O)O]

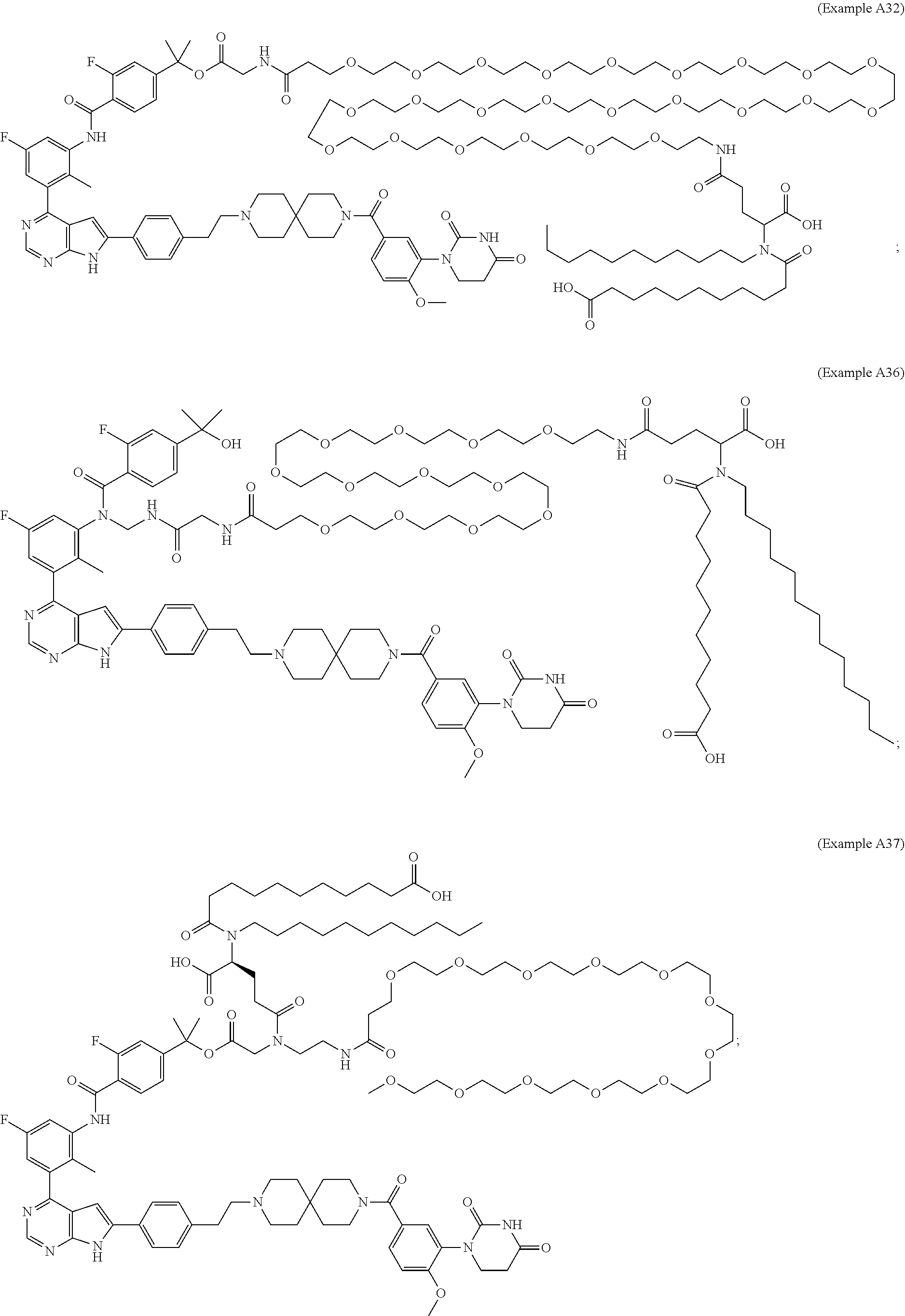

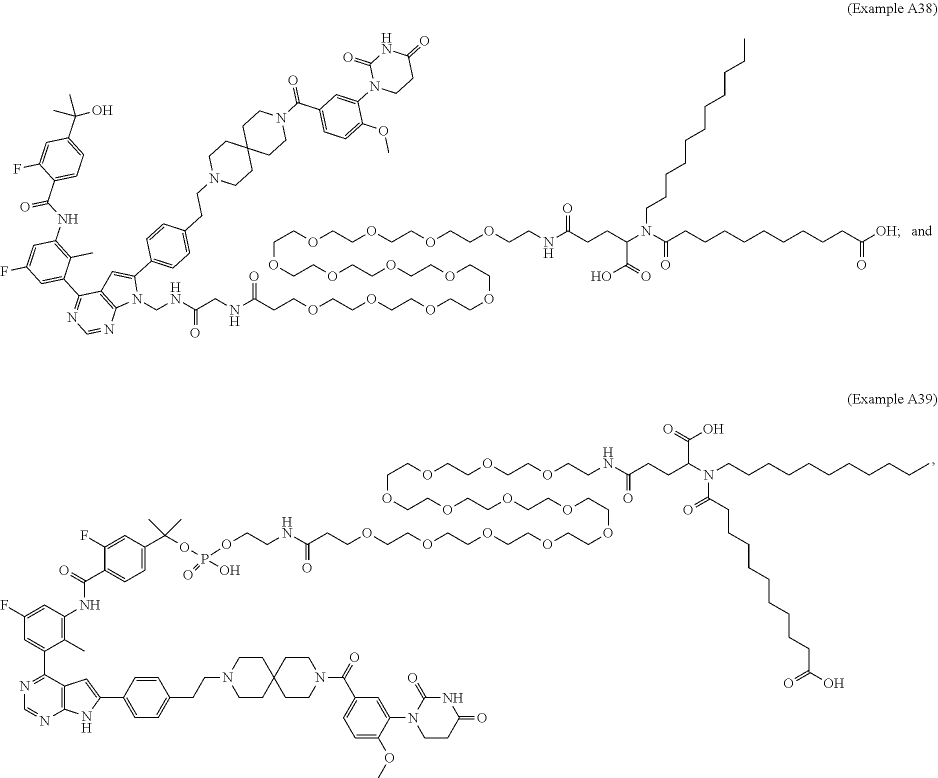 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCC(=O)NCn1c(-c2ccc(CCN3CCC4(CC3)CCN(C(=O)c3ccc(OC)c(N5CCC(=O)NC5=O)c3)CC4)cc2)cc2c(-c3cc(F)cc(NC(=O)c4ccc(C(C)(C)O)cc4F)c3C)ncnc21)C(=O)O ; "and" ; CCCCCCCCCCCN(C(=O)CCCCCCCCCC(=O)O)C(CCC(=O)NCCOCCOCCOCCOCCOCCOCCOCCOCCOCCOCCOCCOCCC(=O)NCCOP(=O)(O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

or a pharmaceutically acceptable salt, hydrate, solvate, prodrug, stereoisomer, or tautomer thereof.

In some embodiments, the fatty acid-bifunctional protein degrader conjugate (e.g., of Formula (I) or (I′)), or a pharmaceutically acceptable salt, hydrate, solvate, prodrug, stereoisomer, or tautomer thereof, is selected from the group consisting of

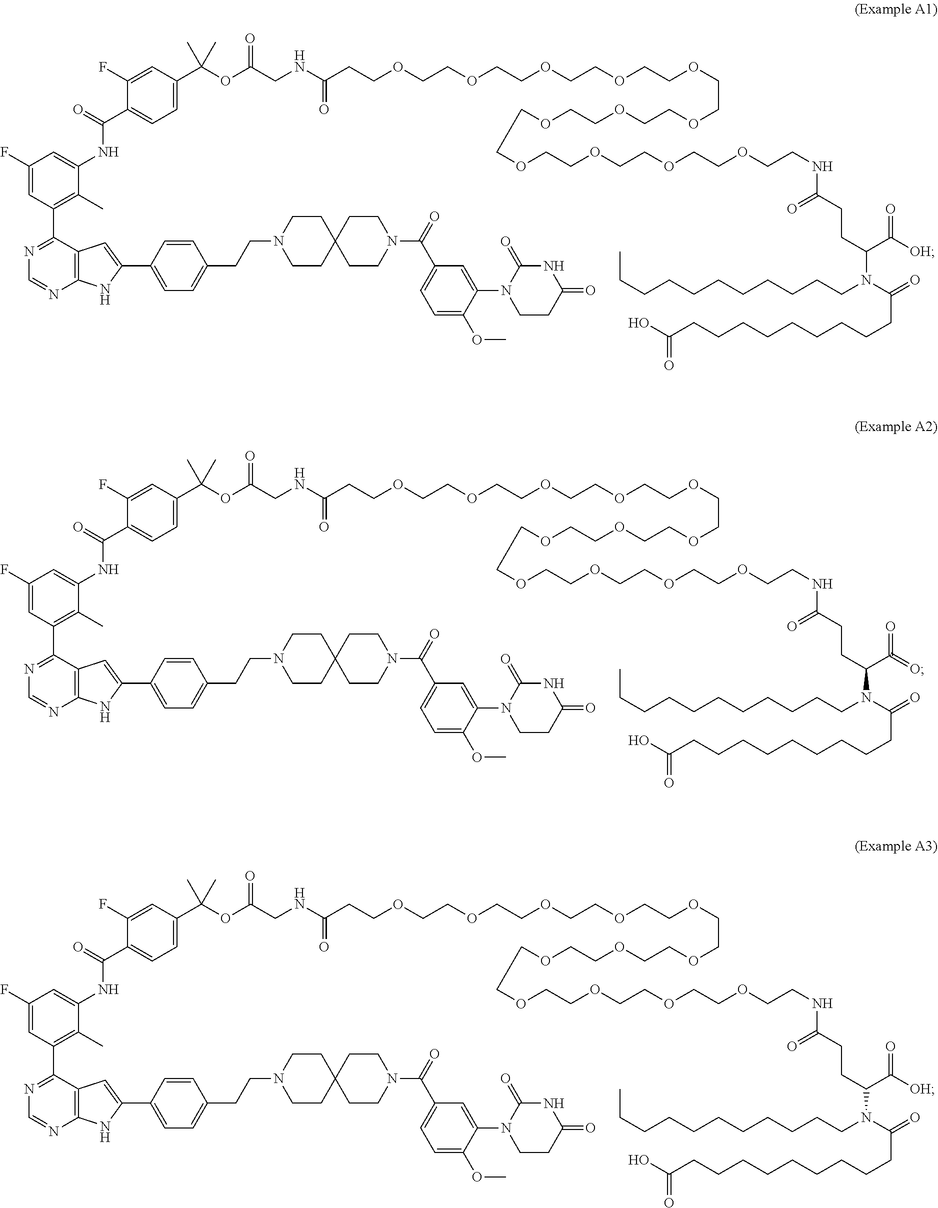

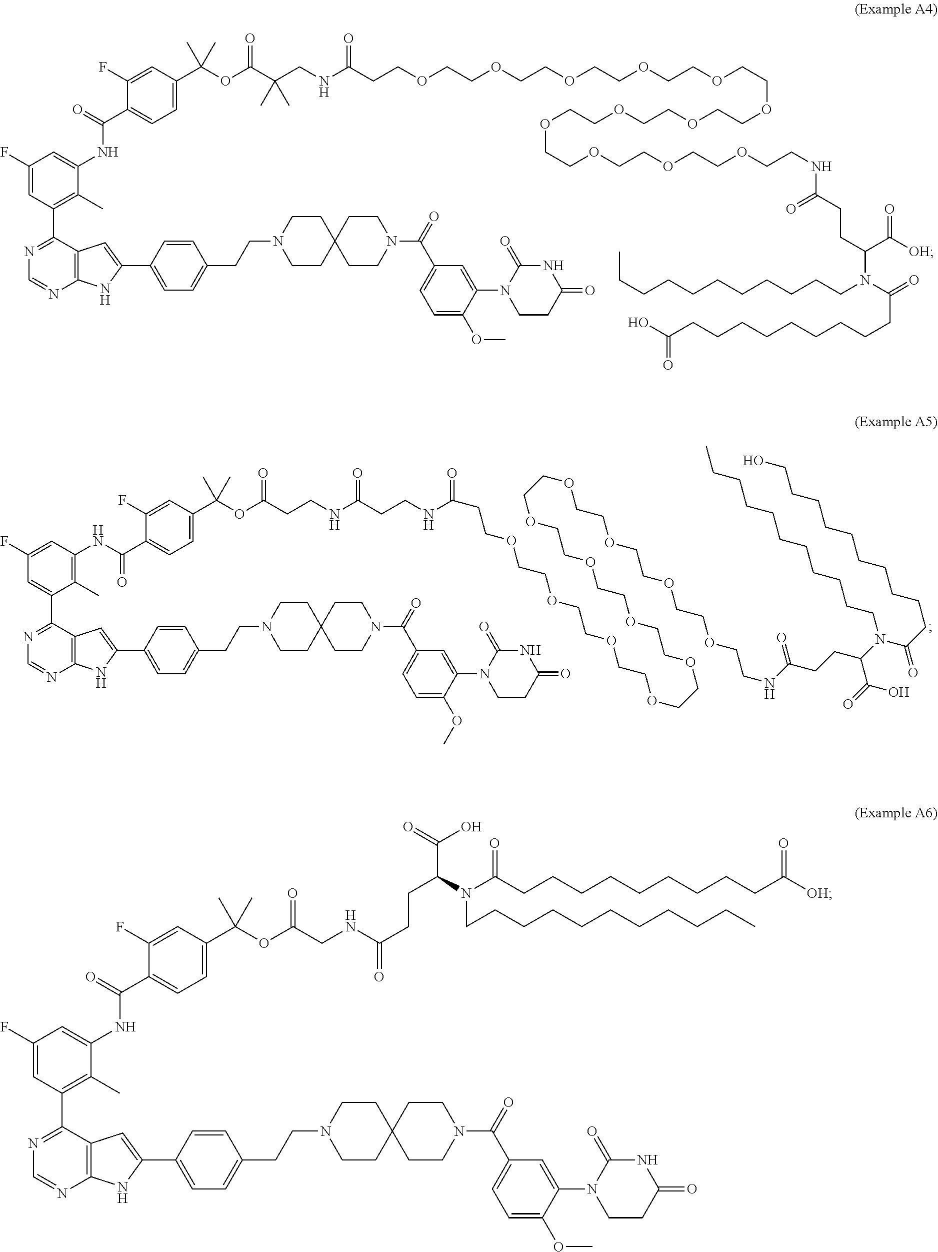

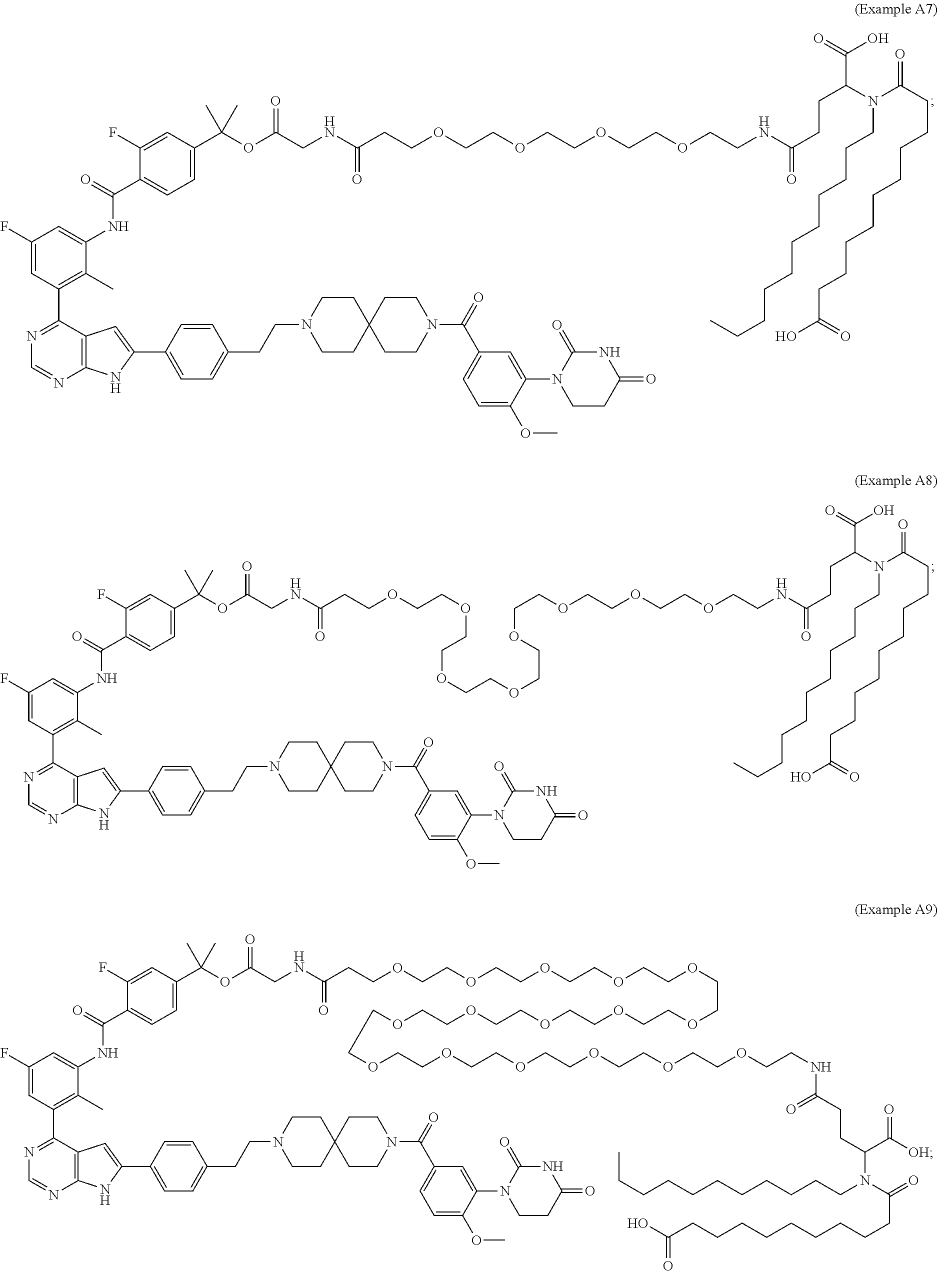

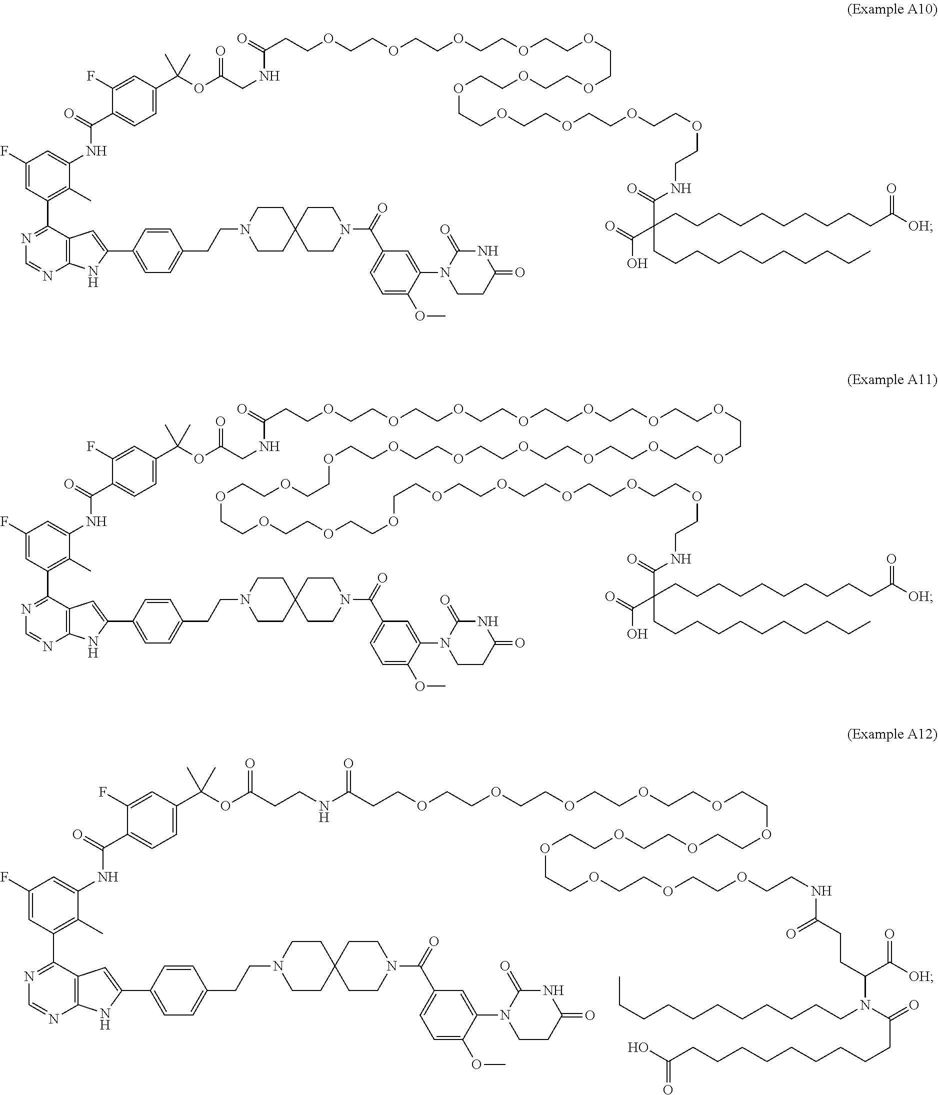

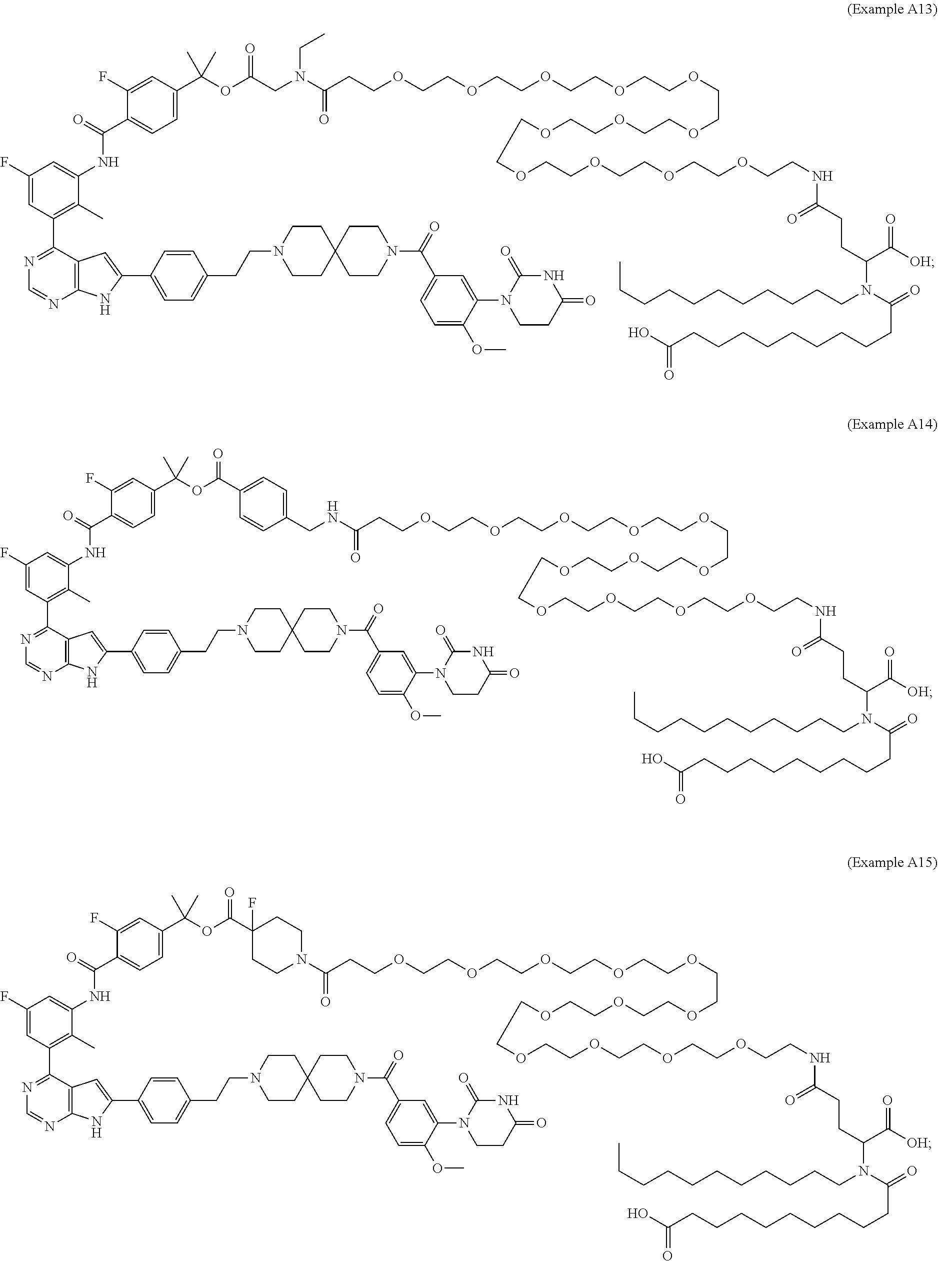

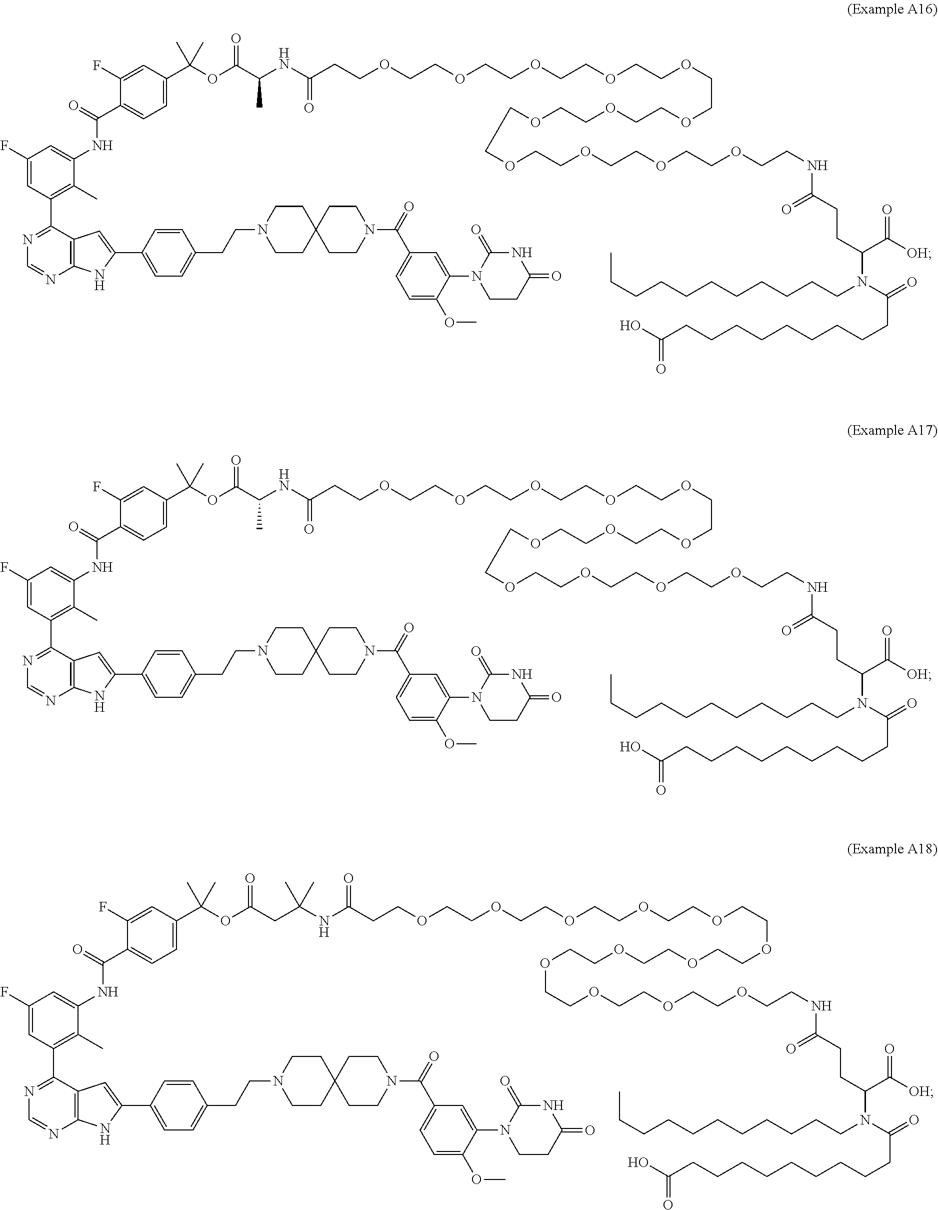

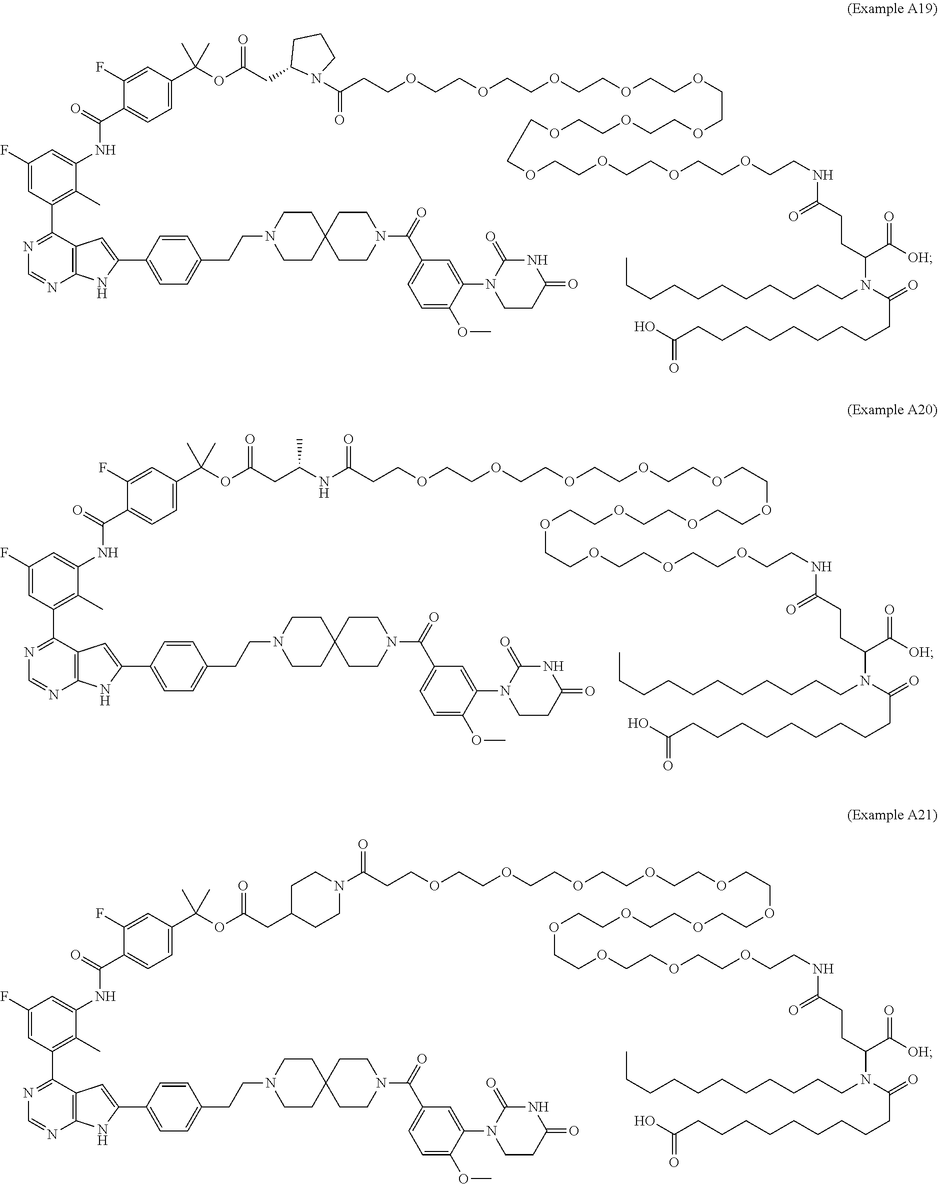

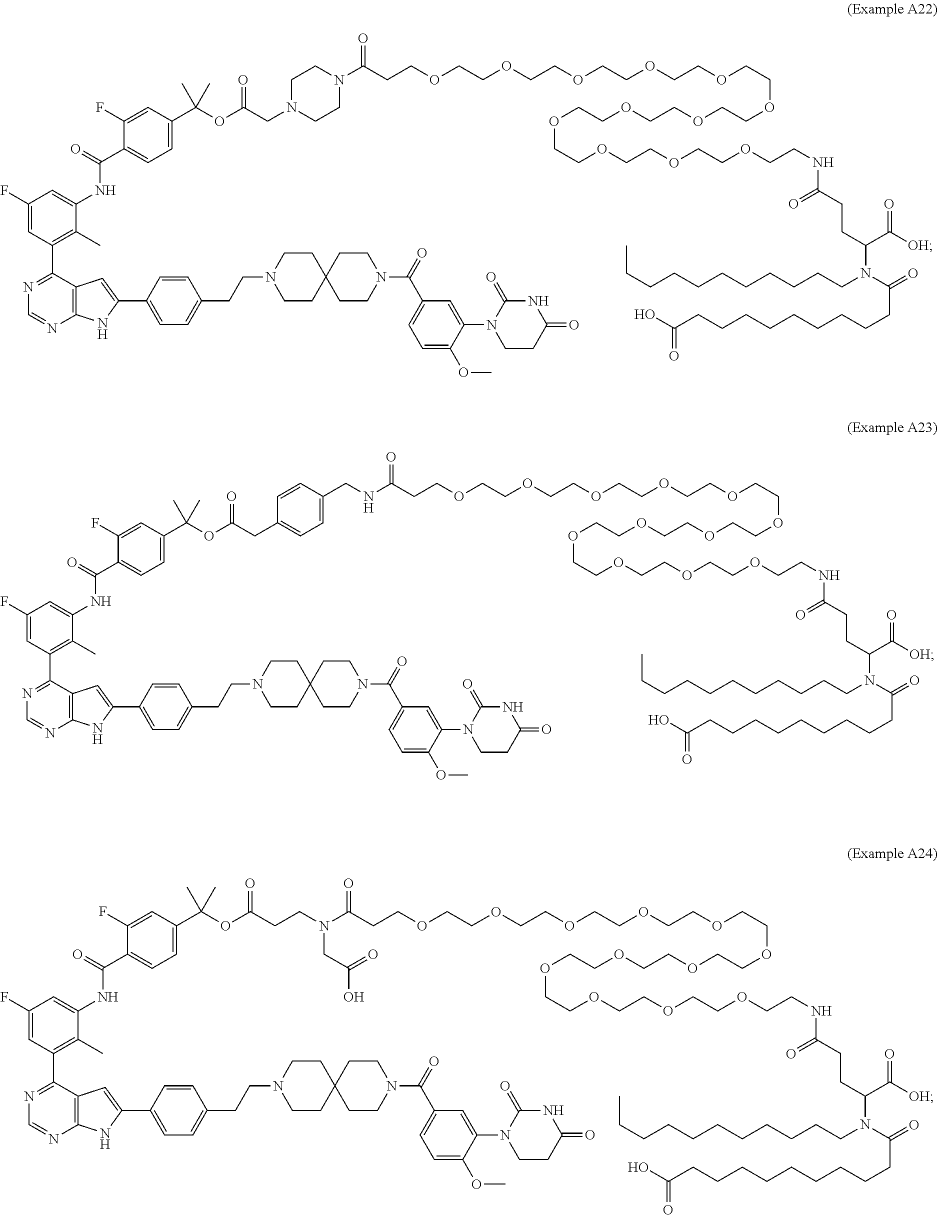

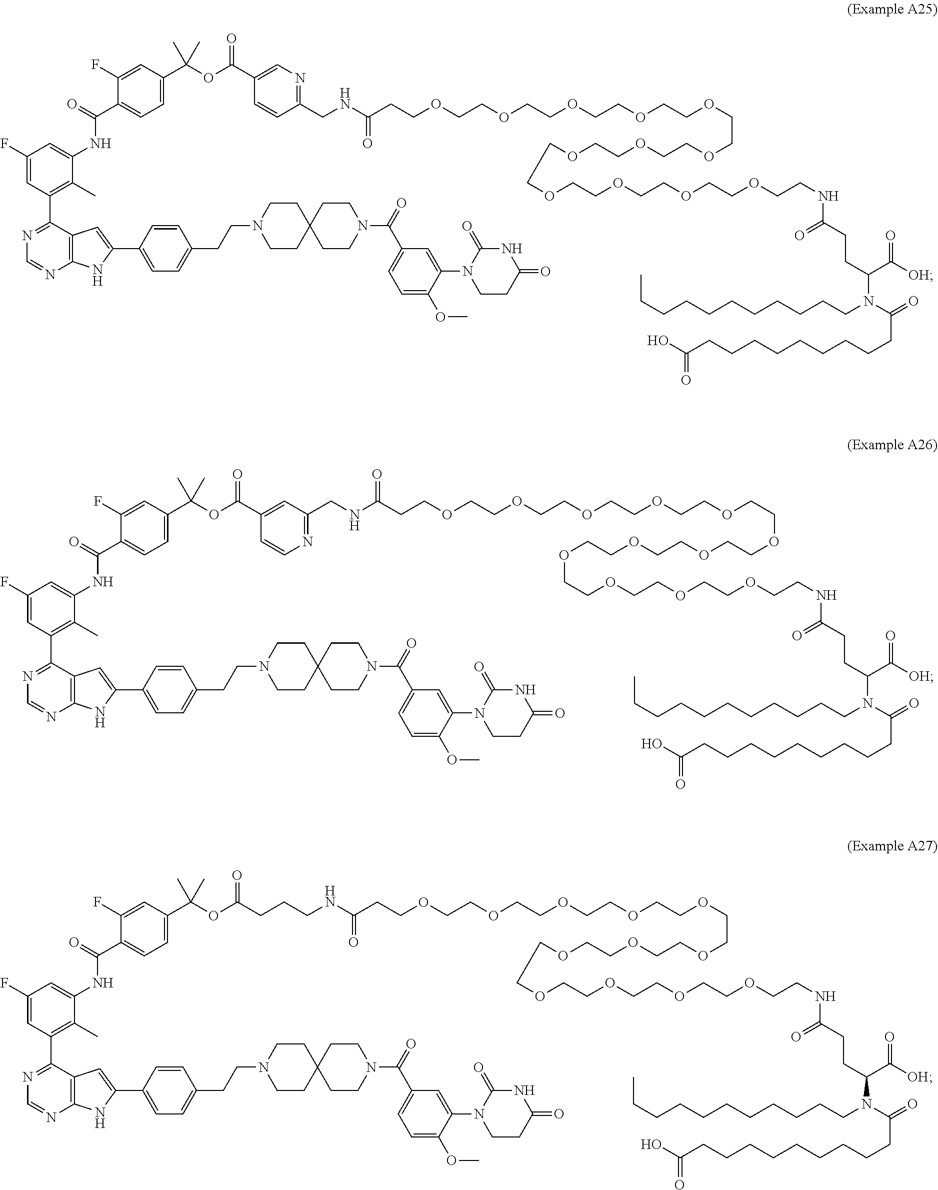

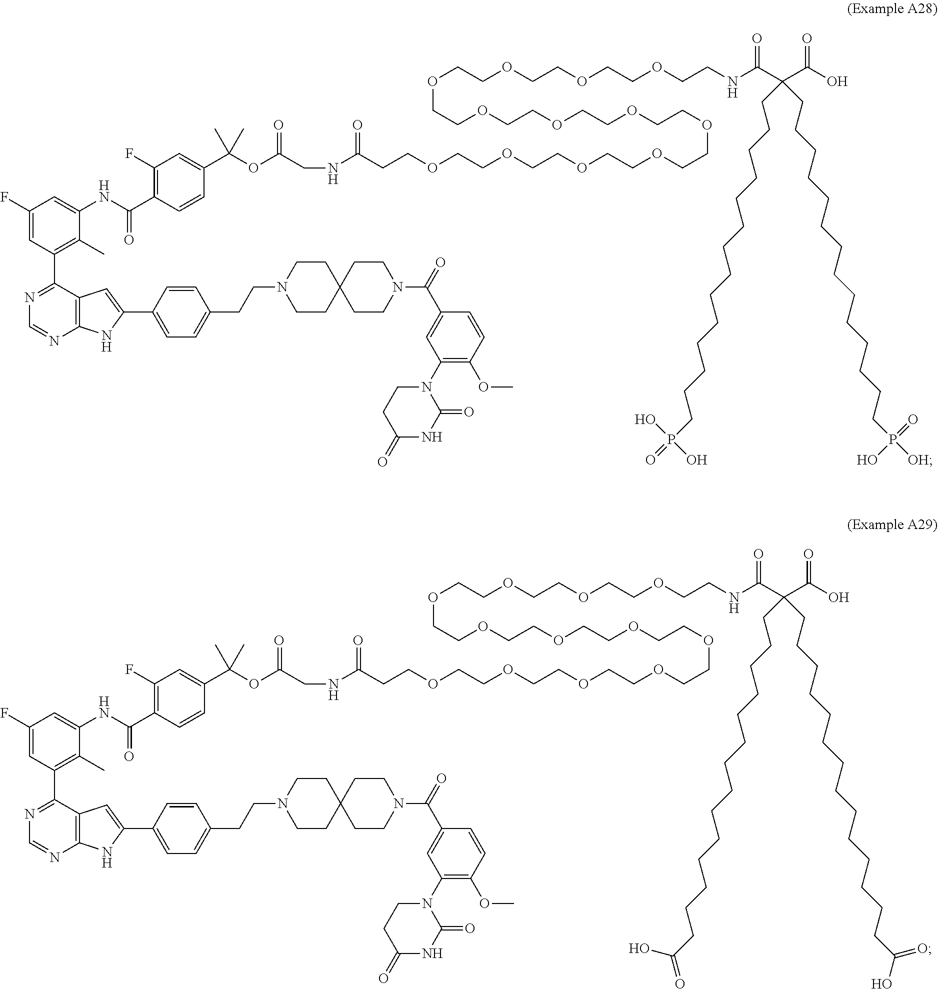 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCNC(=O)C(CCCCCCCCCCCCCCP(=O)(O)O)(CCCCCCCCCCCCCCP(=O)(O)O)C(=O)O)cc8F)c7C)ncnc6[nH]5)cc4)CC3)CC2)cc1N1CCC(=O)NC1=O ; COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCNC(=O)C(CCCCCCCCCCCCCCC(=O)O)(CCCCCCCCCCCCCCC(=O)O)C(=O)O)cc8F)c7C)ncnc6[nH]5)cc4)CC3)CC2)cc1N1CCC(=O)NC1=O]

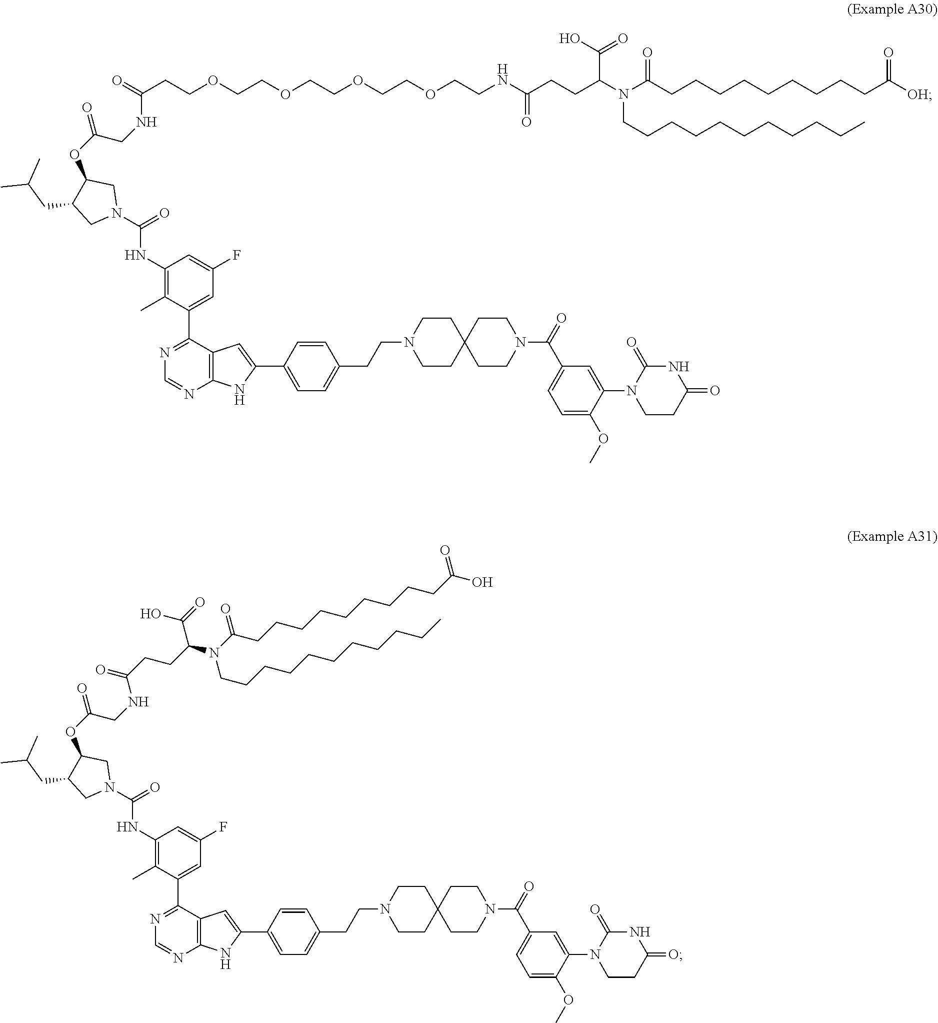 [SMILES: CCCCCCCCCCCN(C(=O)CCCCCCCCCC(=O)O)C(CCC(=O)NCCOCCOCCOCCOCCC(=O)NCC(=O)O[C@H]1CN(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)C[C@@H]1CC(C)C)C(=O)O ; CCCCCCCCCCCN(C(=O)CCCCCCCCCC(=O)O)[C@@H](CCC(=O)NCC(=O)O[C@H]1CN(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)C[C@@H]1CC(C)C)C(=O)O]

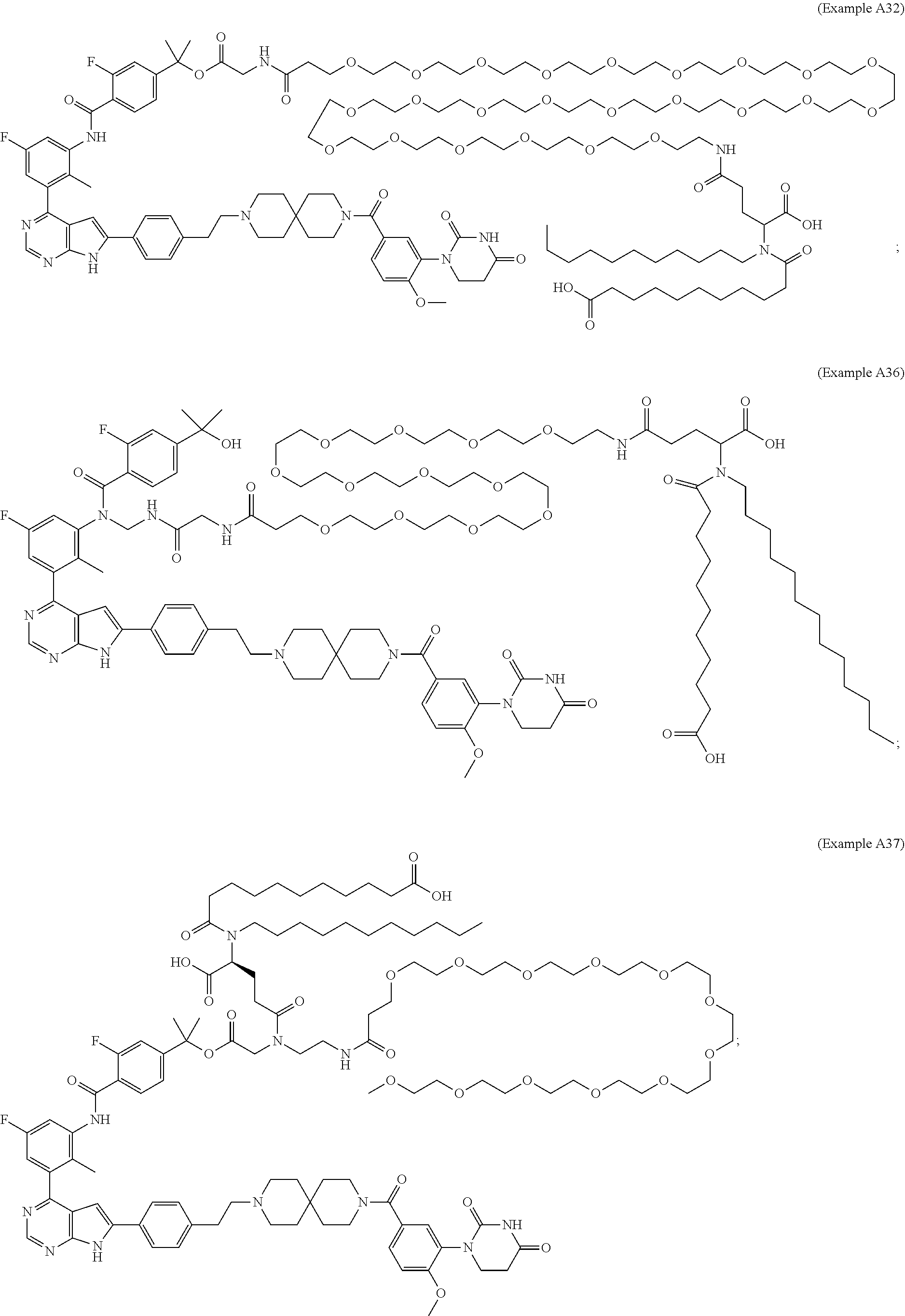

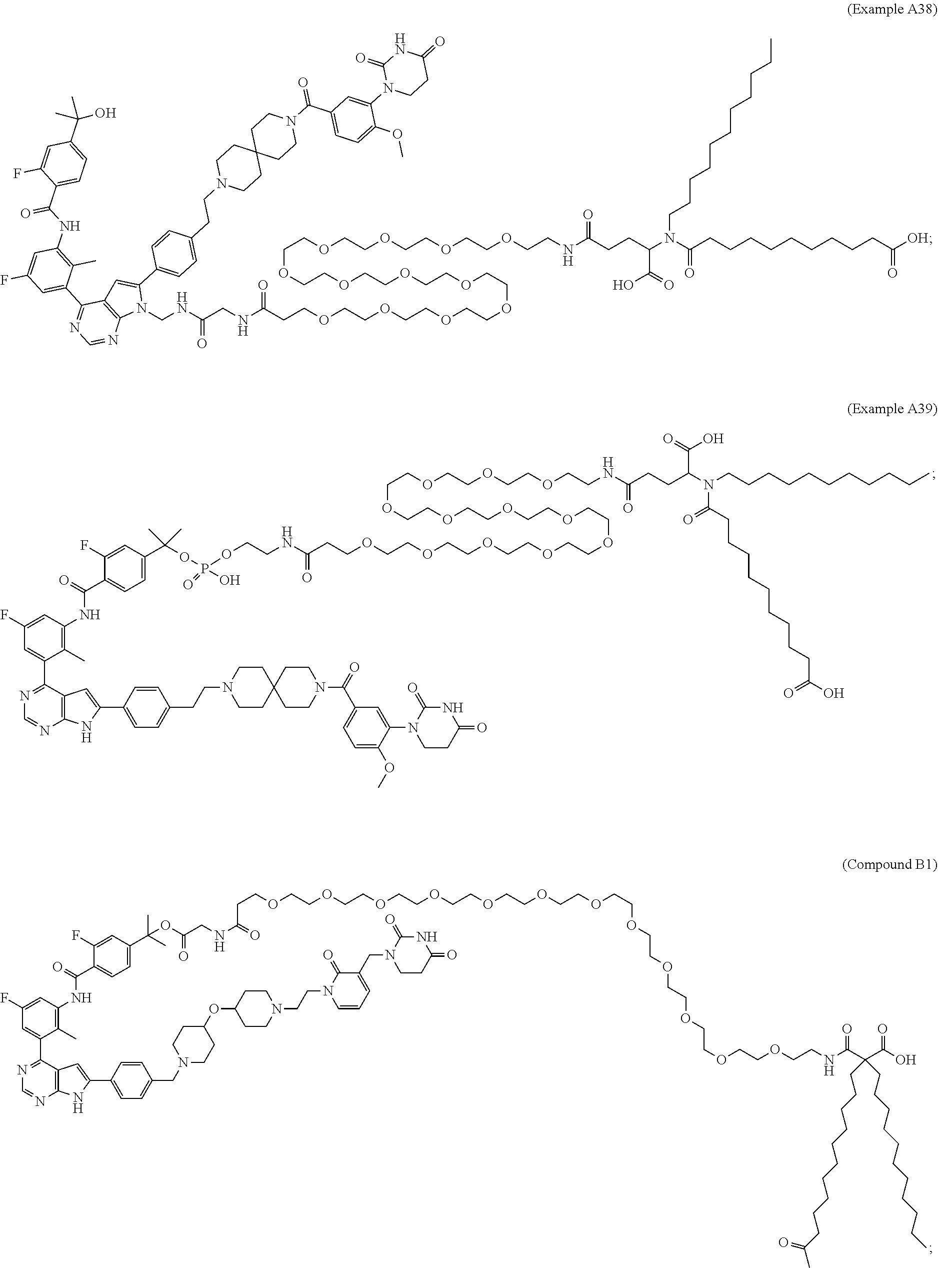

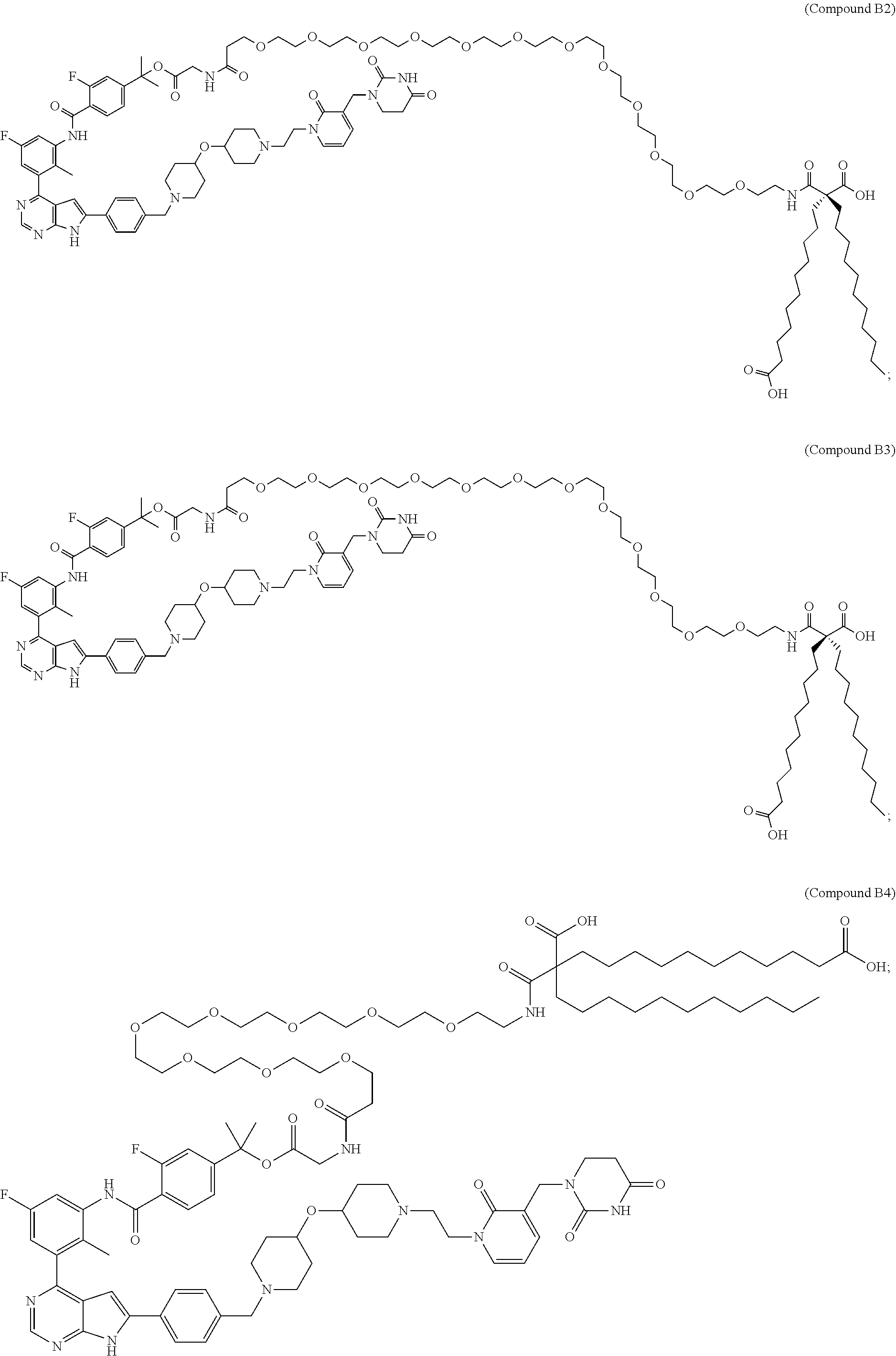

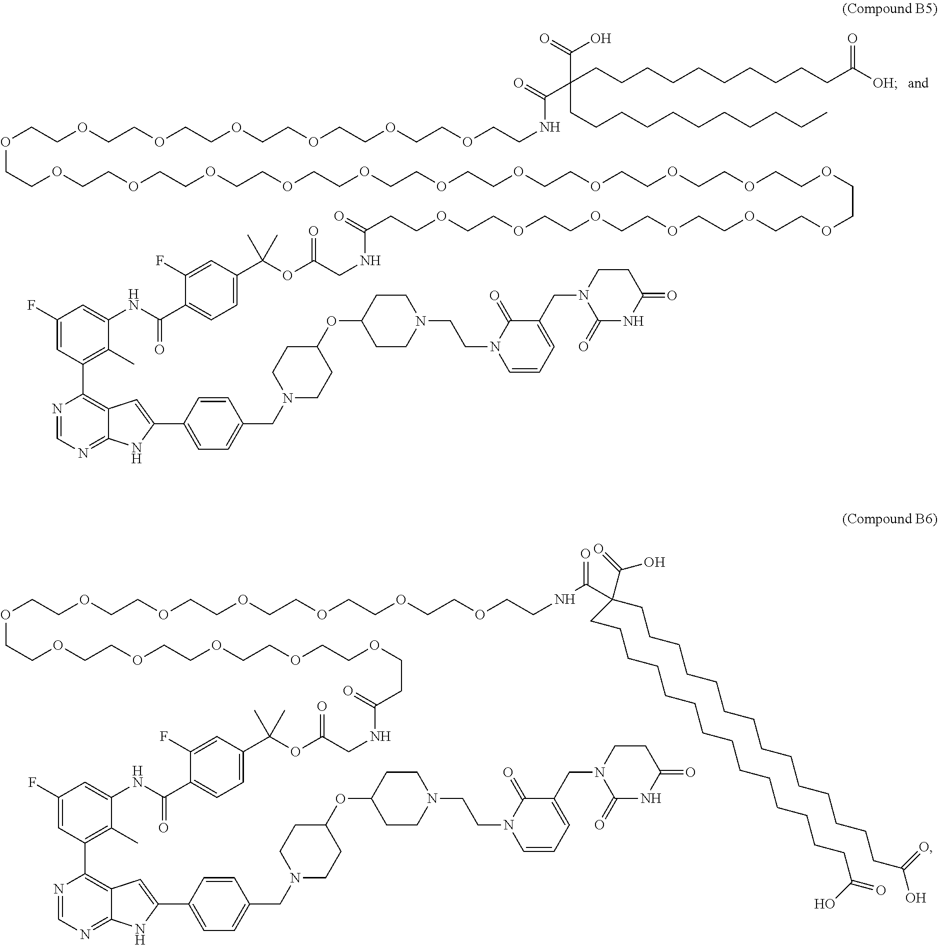 [SMILES: CCCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1 ; "and" ; Cc1c(NC(=O)c2ccc(C(C)(C)OC(=O)CNC(=O)CCOCCOCCOCCOCCOCCOCCOCCOCCOCCOCCOCCOCCNC(=O)C(CCCCCCCCCCCCCCC(=O)O)(CCCCCCCCCCCCCCC(=O)O)C(=O)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

or a pharmaceutically acceptable salt, hydrate, solvate, prodrug, stereoisomer, or tautomer thereof.

In one embodiment, the conjugate, or a pharmaceutically acceptable salt, hydrate, solvate, prodrug, stereoisomer, or tautomer thereof, is selected from the group consisting of:

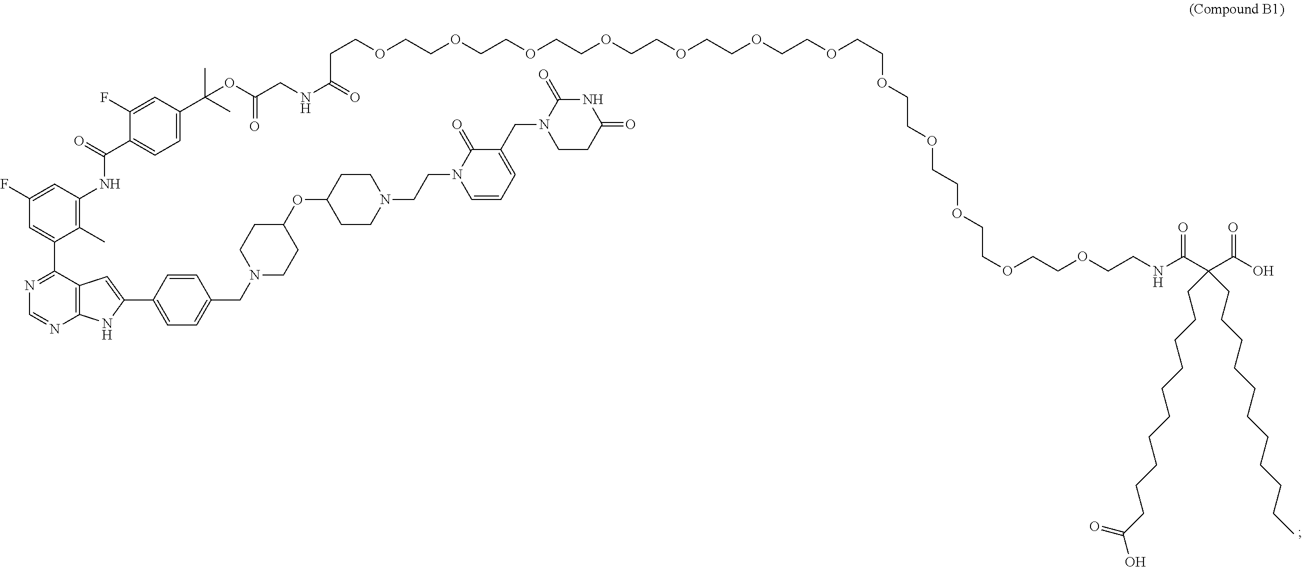 [SMILES: CCCCCCCCCCCC(CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

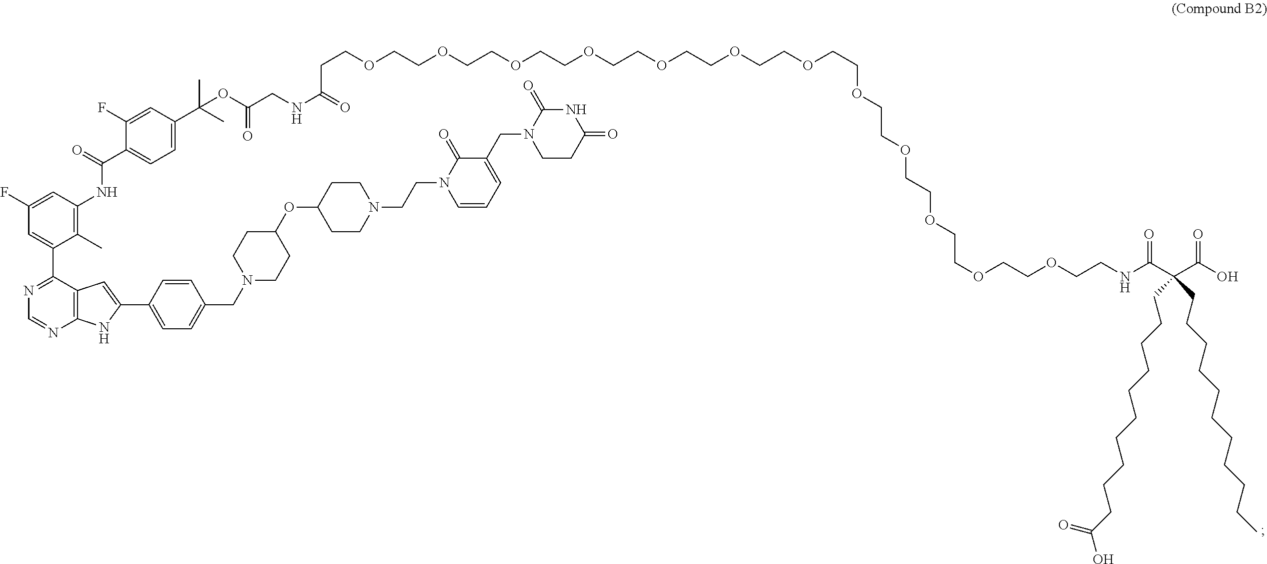 [SMILES: CCCCCCCCCCC[C@](CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

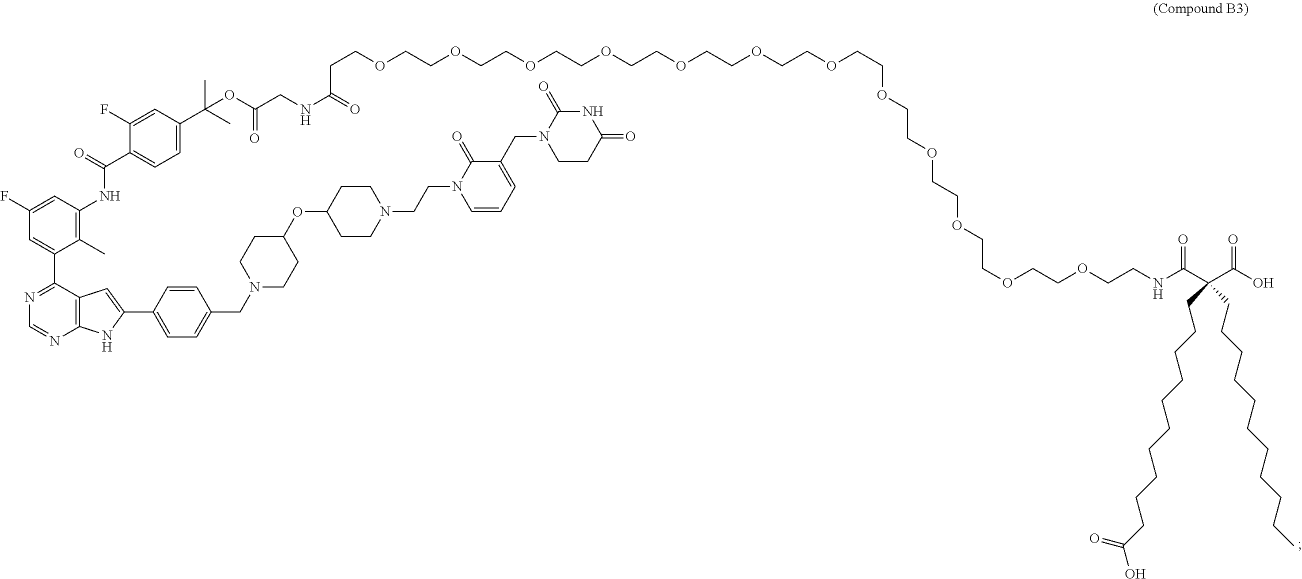 [SMILES: CCCCCCCCCCC[C@@](CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

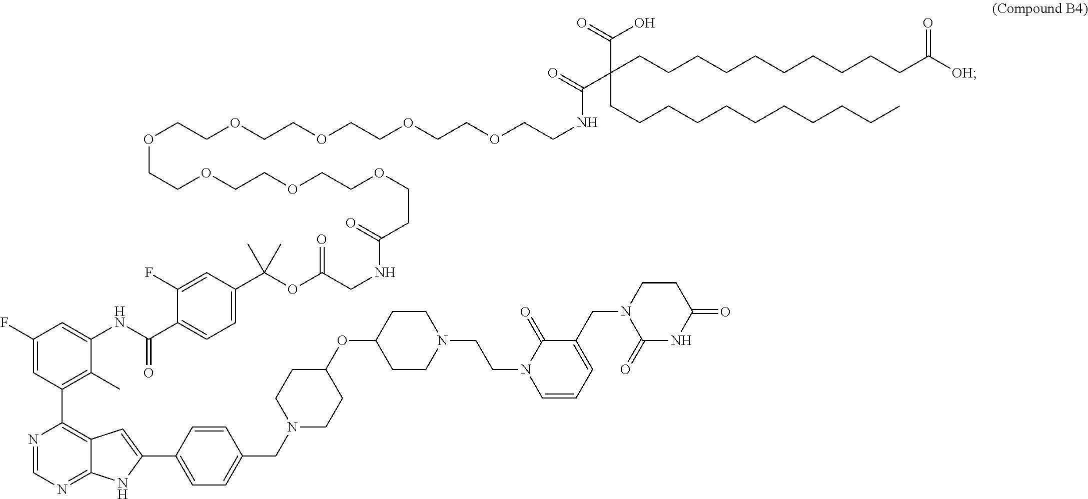 [SMILES: CCCCCCCCCCCC(CCCCCCCCCCC(=O)O)(C(=O)O)C(=O)NCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

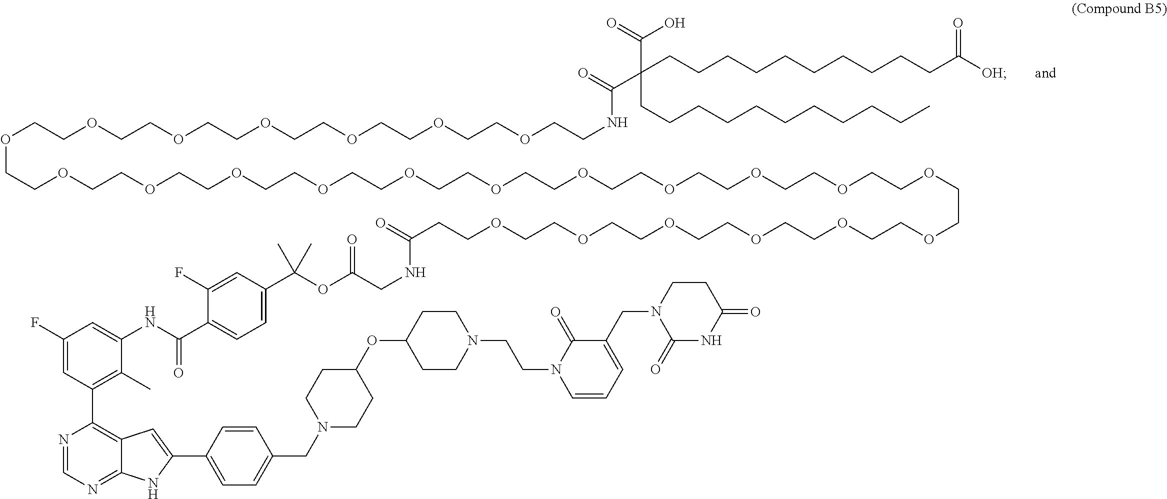 [SMILES: CCCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1 ; "and"]

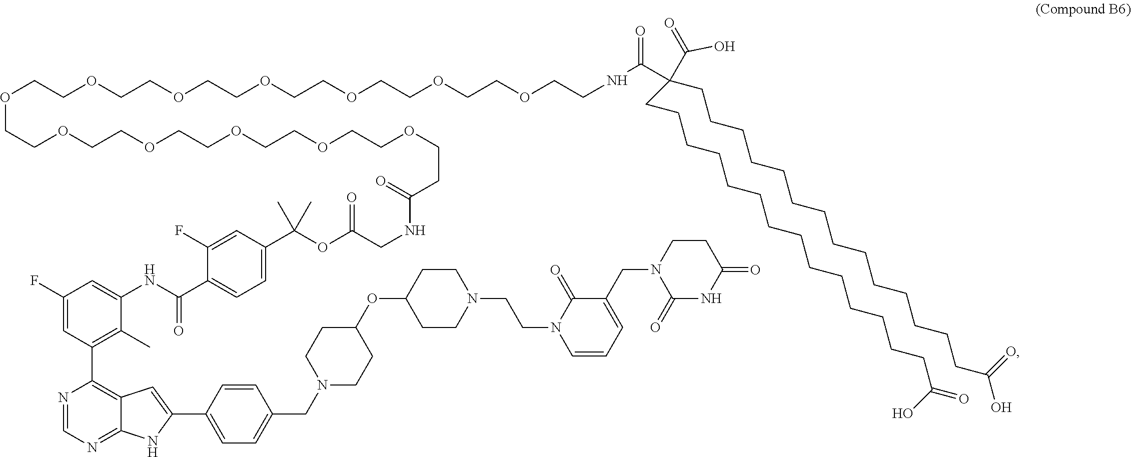 [SMILES: Cc1c(NC(=O)c2ccc(C(C)(C)OC(=O)CNC(=O)CCOCCOCCOCCOCCOCCOCCOCCOCCOCCOCCOCCOCCNC(=O)C(CCCCCCCCCCCCCCC(=O)O)(CCCCCCCCCCCCCCC(=O)O)C(=O)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

or a pharmaceutically acceptable salt, hydrate, solvate, prodrug, stereoisomer, or tautomer thereof.

In an embodiment, the conjugate, or a pharmaceutically acceptable salt, hydrate, solvate, prodrug, stereoisomer, or tautomer thereof, has the following structural formula:

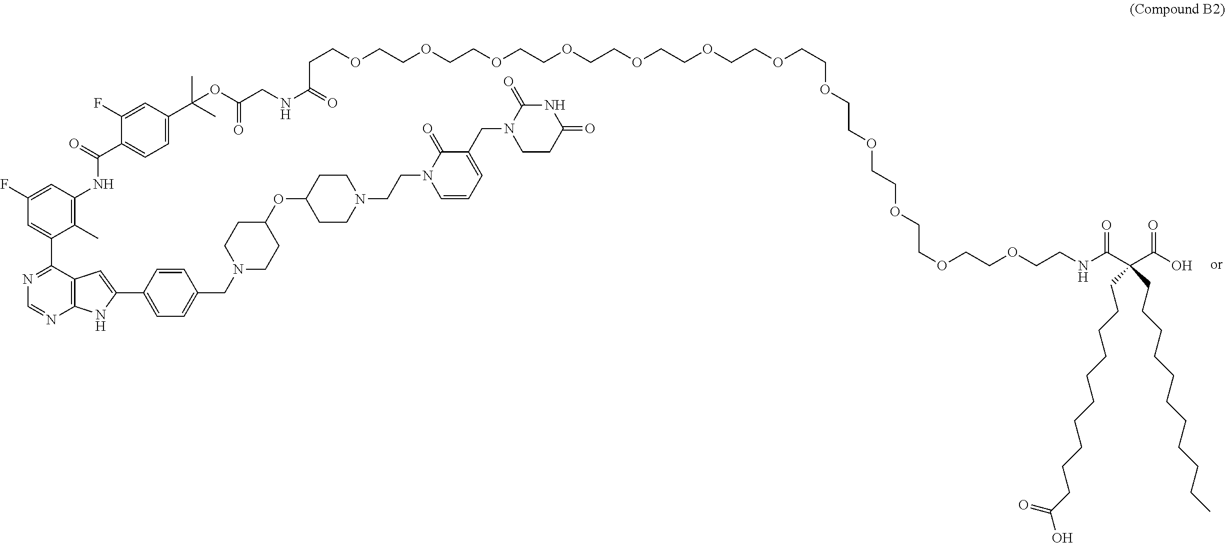 [SMILES: CCCCCCCCCCC[C@](CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1 ; "or"]

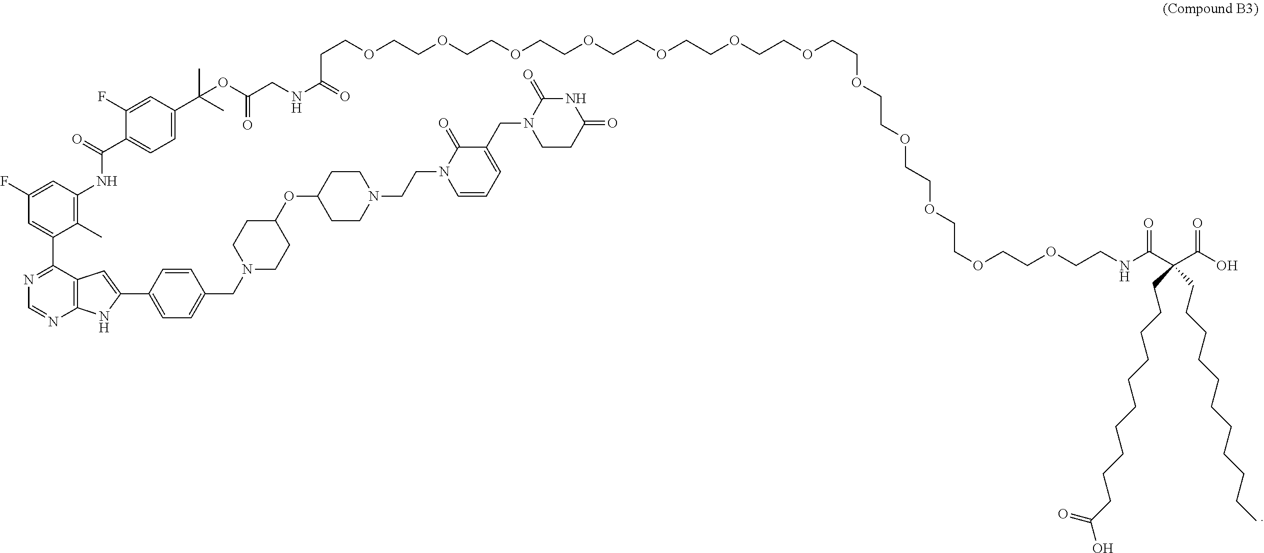 [SMILES: CCCCCCCCCCC[C@@](CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

In an embodiment, the conjugate, or pharmaceutically acceptable salt, solvate, prodrug, stereoisomer, or tautomer thereof, is present as an R-enantiomeric enriched mixture. In an embodiment, the conjugate, or pharmaceutically acceptable salt, solvate, prodrug, stereoisomer, or tautomer thereof, is present as an S-enantiomeric enriched mixture.

In an embodiment, the conjugate, or pharmaceutically acceptable salt, solvate, prodrug, stereoisomer, or tautomer thereof, wherein the improvement of a bifunctional protein degrader AUC compared to the non-conjugated bifunctional protein degrader without i) the linker L1 and optionally a solubilizing domain or L⁴; and ii) the Fatty Acid (FA-1) is at least 2 fold, e.g., at least 5 fold, at least 10 fold, at least 20 fold, or at least 30 fold. The improvement of a bifunctional protein degrader AUC in the conjugates of the disclosure compared to the non-conjugated bifunctional protein degrader without i) the linker L1 and optionally a solubilizing domain or L4; and ii) the Fatty Acid (FA-1) may be at least 2 fold, e.g., at least 5 fold, at least 10 fold, at least 20 fold, at least 30 fold, e.g. in mice, in rats, in dogs, or in primates (e.g., humans). In one embodiment, the improvement of bifunctional protein AUC in the conjugates is at least 2 fold, at least 5 fold, at least 10 fold, at least 20 fold, or at least 30 fold. In another embodiment, the improvement of bifunctional protein degrader AUC in the conjugates is about at least 2 fold, about at least 5 fold, about at least 10 fold, about at least 20 fold, or about at least 30 fold. In yet another embodiment, the improvement of bifunctional protein degrader AUC in the conjugates is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, or about 30 fold. In yet another embodiment, the improvement of bifunctional protein degrader AUC in the conjugates is 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, or 30 fold. In yet another embodiment, the improvement of bifunctional protein degrader AUC in the conjugates is between about 2 fold to about 5 fold, between about 3 fold to about 5 fold, between about 3 fold to about 6 fold, between about 4 fold to about 7 fold, between about 5 fold to about 10 fold, between about 6 fold to about 11 fold, between about 7 fold to about 12 fold, between about 8 fold to about 13 fold, between about 9 fold to about 14 fold, between about 10 fold to about 15 fold, between about 10 fold to about 20 fold, between about 15 fold to about 20 fold, between about 15 fold to about 25 fold, between about 20 fold to about 30 fold, between about 25 fold to about 30 fold, between about 25 fold to about 35 fold, between about 30 fold to about 40 fold, between about 30 fold to about 35 fold, between about 35 fold to about 40 fold, between about 40 fold to about 45 fold, or between about 40 fold to about 50 fold. In yet another embodiment, the improvement of bifunctional protein degrader AUC in the conjugates is 2 fold, 5 fold, 10 fold, 20 fold, or 30 fold.

The decrease in Cmax in the conjugates of the disclosure compared to the non-conjugated bifunctional protein degraders without i) the linker L1 and optionally a solubilizing domain or L⁴; and ii) the Fatty Acid (FA-1), e.g. in mice, in rats, in dogs or in primates (e.g. humans) may be at least 2 fold, e.g. at least 5 fold, e.g. at least 10 fold. In one embodiment, the decrease in Cmax in the conjugates is at least 2 fold, at least 5 fold, at least 10 fold. In another embodiment, the decrease in Cmax in the conjugates is about at least 2 fold, about at least 5 fold, about at least 10 fold. In another embodiment, the decrease in Cmax in the conjugates is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, or about 30 fold, In another embodiment, the decrease in Cmax in the conjugates is 2 fold, 3 fold, 4 fold, 5 fold, 6 fold, 7 fold, 8 fold, 9 fold, 10 fold, 11 fold, 12 fold, 13 fold, 14 fold, 15 fold, 6 fold, 17 fold, 18 fold, 19 fold, 20 fold, 21 fold, 22 fold, 23 fold, 24 fold, 25 fold, 26 fold, 27 fold, 28 fold, 29 fold, or 30 fold,

In yet another embodiment, the decrease in Cmax in the conjugates is between about 2 fold to about 5 fold, between about 3 fold to about 5 fold, between about 3 fold to about 6 fold, between about 4 fold to about 7 fold, between about 5 fold to about 10 fold, between about 6 fold to about 11 fold, between about 7 fold to about 12 fold, between about 8 fold to about 13 fold, between about 9 fold to about 14 fold, between about 10 fold to about 15 fold, between about 10 fold to about 20 fold, between about 15 fold to about 20 fold, between about 15 fold to about 25 fold, between about 20 fold to about 30 fold, between about 25 fold to about 30 fold, between about 25 fold to about 35 fold, between about 30 fold to about 40 fold, between about 30 fold to about 35 fold, between about 35 fold to about 40 fold, between about 40 fold to about 45 fold, or between about 40 fold to about 50 fold. In yet another embodiment, the decrease in Cmax in the conjugates is 2 fold, 5 fold, 10 fold, 20 fold, or 30 fold.

### Definitions

One embodiment is a compound of any of the formulae described herein, e.g., a compound of Formula (I), (I′), and subformulas thereof, or a pharmaceutically acceptable salt, hydrate, solvate, prodrug, stereoisomer, or tautomer thereof, that modulates, e.g., decreases the amount of a targeted protein or protein of interest, e.g., one or more proteins from Table 1 or Table 2.

Another embodiment is a Formula (I), (I′), and subformulas thereof, or a pharmaceutically acceptable salt, hydrate, solvate, prodrug, stereoisomer, or tautomer thereof, that degrades a targeted protein through the ubiquitin-proteasome pathway (UPP).

The formation of a viable ternary complex among the target protein, the bifunctional degrader, and the E3 ligase substrate receptor is enabled by the use of targeted bifunctional degraders, relying on three components, the “target ligand” or “targeting ligand” and the “target ligase binder” or “targeting ligase binder” (also termed “warheads”) and the joining segment, termed the “linker.” The likelihood that a bifunctional degrader may form an energetically favored viable complex can be assessed using an in silico computational approach. Energetic unfavorability can arise through enthalpic contributions (steric or electronic clashes between the protein targets and the degrader), entropic contributions (reduction in the degrees of freedom upon formation of the ternary complex), or a combination of the two. Using in silico methods, unfavorable linkers can be quickly identified and deprioritized. Various methods have been described for designing bifunctional degraders. See Drummond and Williams, J. Chem. Inf. Model. 59:1634-1644 (2019). The in silico ternary complex modelling protocol consists of four steps (see FIG. 2): (1) generate the conformational ensemble of the bifunctional degraders. For this task, various conformational searches methods available in standard modelling programs can be used. (2) Rigidly superimpose using the coordinates one of the warheads (either the “targeting ligand” or the “targeting ligase binder”) with the same warhead bound in the binary complex structure, as observed in crystal structure or by docking it in the respective protein. (3) Filter to retain sterically competent conformations of the bifunctional degrader with the first protein. (4) Rigidly superimpose the warhead bound in the other binary complex structure to the coordinates of the corresponding warhead in the degrader. Conformations of the targeted bifunctional degraders causing serious clashes between any of the three components of the ternary complexes are filtered out. The saved generated conformations could be further clustered and refined using standard molecular dynamics approaches. This allows the relaxation of minor steric clashes and electrostatic mismatches. It also gives an indication of the stability of the ternary complex; linkers that do not bring the proteins into contact can be said to be entropically disfavored.

For example, the method described herein has been applied to Compounds 01 and 02. Despite both compounds binding to CRBN (Table 4 in PCT/IB2020/058535), Compound 2 enabled the ternary complex formation according to the method described herein, and as such degrades BTK (>95%). However, Compound 01 was predicted to not form a ternary complex and experimentally no degradation of BTK was observed (Table 4 in PCT/IB2020/058535).

It is also possible to design modified linkers using de novo or generative methods to enhance physicochemical properties or some other scoring metric. See Ertl and Lewis, J. Comput Aided Mol. Des. 26(11): 1207-1215 (2012). It is possible to combine both the assessment for ternary complex formation and having favorable properties, to identify an optimal linker space.

The term “a therapeutically effective amount” of a compound described herein refers to an amount of the compound described herein that will elicit the biological or medical response of a subject, for example, reduction inhibition, or degradation of an enzyme or a protein activity, or ameliorate symptoms, alleviate conditions, slow or delay disease progression, or prevent a disease, etc.

In one embodiment, the term “effective amount” or “a therapeutically effective amount” refers to the amount of a conjugate according to the disclosure that, when administered to a subject, is effective to (1) at least partially alleviate, prevent and/or ameliorate a condition, or a disorder or a disease (i) mediated by a target protein, (ii) associated with activity of a target protein, or (iii) characterized by activity (normal or abnormal) of a target protein, or (iv) modulated by activity of a target protein; or (2) reduce or inhibit the activity of a target protein; or (3) reduce or inhibit the expression of a target protein; or (4) degrade a target protein. These effects may be achieved for example by reducing the amount of a target protein by degrading of the target protein. In one embodiment, the term “a therapeutically effective amount” refers to the amount of a conjugate of the disclosure that, when administered to a cell, or a tissue, or a non-cellular biological material, or a medium, is effective to at least partially reduce or inhibit the activity of target protein; or at least partially reduce or inhibit the expression of a target protein, for example by degrading a target protein.

As used herein, the term cancer refers to a neoplastic disease and includes for instance solid tumors, such as, e.g. sarcomas or carcinomas or blood cancer, such as, e.g. leukemia or myeloma, or cancers of lymphatic system such as lymphoma, or mixed types thereof.

As used herein, the terms “degrades”, “degrading”, or “degradation” refers to the partial or full breakdown of a target protein by the cellular proteasome system to an extent that reduces or eliminates the biological activity (especially aberrant activity) of target protein. Degradation may be achieved through mediation of an E3 ligase, in particular, E3-ligase complexes comprising the protein Cereblon. As used herein, the term “modulation of target protein activity” or “modulating target activity” means the alteration of, especially reduction, suppression or elimination, of target protein's activity. This may be achieved by degrading the target protein in vivo or in vitro. The amount of target protein degraded can be measured by comparing the amount of target protein remaining after treatment with a compound described herein as compared to the initial amount or level of target protein present as measured prior to treatment with a compound described herein. In an embodiment, at least about 30% of the target protein is degraded compared to initial levels. In an embodiment, at least about 40% of the target protein is degraded compared to initial levels. In an embodiment, at least about 50% of the target protein is degraded compared to initial levels. In an embodiment, at least about 60% of the target protein is degraded compared to initial levels. In an embodiment, at least about 70% of the target protein is degraded compared to initial levels. In an embodiment, at least about 80% of the target protein is degraded compared to initial levels. In an embodiment, at least about 90% of the target protein is degraded compared to initial levels. In an embodiment, at least about 95% of the target protein is degraded compared to initial levels. In an embodiment, over 95% of the target protein is degraded compared to initial levels. In an embodiment, at least about 99% of the target protein is degraded compared to initial levels.

In an embodiment, the target protein is degraded in an amount of from about 30% to about 99% compared to initial levels. In an embodiment, the target protein is degraded in an amount of from about 40% to about 99% compared to initial levels. In an embodiment, the target protein is degraded in an amount of from about 50% to about 99% compared to initial levels. In an embodiment, the target protein is degraded in an amount of from about 60% to about 99% compared to initial levels. In an embodiment, the target protein is degraded in an amount of from about 70% to about 99% compared to initial levels. In an embodiment, the target protein is degraded in an amount of from about 80% to about 99% compared to initial levels. In an embodiment, the target protein is degraded in an amount of from about 90% to about 99% compared to initial levels. In an embodiment, the target protein is degraded in an amount of from about 95% to about 99% compared to initial levels. In an embodiment, the target protein is degraded in an amount of from about 90% to about 95% compared to initial levels.

As used herein, the term “selectivity for the target protein” means, for example, a compound described herein degrades the target protein in preference to, or to a greater extent than, another protein or proteins.

As used herein, the term “subject” refers to an animal. Typically, the animal is a mammal. A subject also refers to, for example, primates (e.g., humans, male or female), cows, sheep, goats, horses, dogs, cats, rabbits, guinea pigs, pigs, rats, mice, fish, birds, and the like. In an embodiment, the subject is a primate. In a preferred embodiment, the subject is a human.

As used herein, the terms “inhibit”, “inhibition”, or “inhibiting” refer to the reduction or suppression of a given condition, symptom, or disorder, or disease, or a significant decrease in the baseline activity of a biological activity or process.

As used herein, the terms “treat”, “treating”, or “treatment” of any disease or disorder refer In an embodiment, to ameliorating the disease or disorder (i.e., slowing or arresting or reducing the development of the disease or at least one of the clinical symptoms thereof). In an embodiment, “treat”, “treating”, or “treatment” refers to alleviating or ameliorating at least one physical parameter including those which may not be discernible by the patient.

As used herein, the term “prevent”, “preventing”, or “prevention” of any disease or disorder refers to the prophylactic treatment of the disease or disorder; a reduction in the frequency of, or delay in the onset or progression of the disease or disorder, for example, symptoms of the condition.

As used herein, a subject is “in need of” a treatment if such subject would benefit biologically, medically, or in quality of life from such treatment.

As used herein, the term “a,” “an,” “the” and similar terms used in the context of the disclosure (especially in the context of the claims) are to be construed to cover both the singular and plural unless otherwise indicated herein or clearly contradicted by the context.

The term “alkyl” refers to a radical of a straight-chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tertiary amyl), and hexyl (C₆) (e.g., n-hexyl).

“Alkylene” refers to a divalent radical of an alkyl group, e.g., —CH₂—, —CH₂CH₂—, and —CH₂CH₂CH₂—.

As used herein, the term “alkoxyalkyl” refers to an alkylene, as defined herein, substituted with an alkoxy group, as defined herein, e.g. —CH₂—O—CH₂CH₃. The term “C₁-C₆ alkoxyalkyl” as used herein is equivalent to “C₁-C₆alkoxyC₁₋₆alkyl”. Thus, it includes substituents of the general formula —(CH₂)₁₋₆—O—(CH₂)₁₋₅—CH₃ and branched equivalents thereof.

“Alkenylene” means a straight or branched bivalent hydrocarbon chain containing at least one carbon-carbon double bond. Representative alkenylene groups include —CH═CH—, —CH₂CH═CH—, —C(CH₃)═CH—, —CH₂CH═CHCH₂—, and the like.

“Alkynylene” means a straight or branched bivalent hydrocarbon chain containing at least one carbon-carbon triple bond. Representative alkynylene include —CH≡CH—, —CH≡C—CH₂—, —CH≡C—CH(CH₃)—, and the like.

“Heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁₋₃ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

“Heteroalkylene” refers to a divalent radical of a heteroalkyl group.

“Alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “aryl” refers to a stable, aromatic, mono- or bicyclic ring radical having the specified number of ring carbon atoms. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

As used herein, the term “heteroaryl” refers to a stable, aromatic, mono- or bicyclic ring radical having the specified number of ring atoms and comprising one or more heteroatoms individually selected from nitrogen, oxygen and sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen and sulfur.

As used herein, the term “carbocyclyl” refers to a stable, saturated or unsaturated, non-aromatic, mono- or bicyclic (fused, bridged, or spiro) ring radical having the specified number of ring carbon atoms. Examples of carbocyclyl groups include, but are not limited to, the cycloalkyl groups identified above, cyclobutenyl, cyclopentenyl, cyclohexenyl, and the like. In an embodiment, the specified number is C₃-C₁₂ carbons. The related term “carbocyclic ring” likewise refers to a stable, saturated or unsaturated, non-aromatic, mono- or bicyclic (fused, bridged, or spiro) ring having the specified number of ring carbon atoms. In an embodiment, the carbocyclyl can be substituted or unsubstituted. In an embodiment, the carbocyclyl can be substituted with 0-4 occurrences of R^{a}, wherein each R^{a} is independently selected from the group consisting of C₁₋₆ alkyl, C₁₋₆ alkoxyl, and halogen.

As used herein, the term “heterocyclyl” refers to a stable, saturated or unsaturated, non-aromatic, mono- or bicyclic (fused, bridged, or spiro) ring radical having the specified number of ring atoms and comprising one or more heteroatoms individually selected from nitrogen, oxygen and sulfur. The heterocyclyl radical may be bonded via a carbon atom or heteroatom. In an embodiment, the specified number is C₃-C₁₂ carbons. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocyclic ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur. In an embodiment, the heterocyclyl can be substituted or unsubstituted. In an embodiment, the heterocyclyl can be substituted with 0-4 occurrences of R^{a}, wherein each R^{a} is independently selected from the group consisting of C₁₋₆ alkyl, C₁₋₆ alkoxyl, and halogen.

As used herein, “spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. For example, a (C₃-C₁₂)spirocycloalkyl is a spirocycle containing between 3 and 12 carbon atoms.

As used herein, “spiroheterocycloalkyl” or “spiroheterocyclyl” means a spirocycle wherein at least one of the rings is a heterocycle wherein one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

As used herein, “halo” or “halogen” refers to fluorine (fluoro, —F), chlorine (chloro, —Cl), bromine (bromo, —Br), or iodine (iodo, —I).

As used herein, “haloalkyl” means an alkyl group substituted with one or more halogens. Examples of haloalkyl groups include, but are not limited to, trifluoromethyl, difluoromethyl, pentafluoroethyl, and trichloromethyl.

As used herein, “substituted”, whether preceded by the term “optionally” or not, means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

The term “unsubstituted” means that the specified group bears no substituents.

The term “polyethylene glycol” as used herein refers to a group of the formula

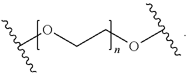

n may be, for example, from 1 to 50, for example 1 to 35, for example, 5 to 30, for example 6 to 25, for example, 6 to 20, for example, 2 to 20, for example, 5 to 15, for example 7 to 15, for example 9 to 13, for example 11.

As used herein, “prodrug” means a compound that, after administration to a subject, is metabolized into a pharmacologically active compound. For example, a prodrug is an amide or an ester of any of the compounds disclosed herein.

As used herein, the definition of each expression, e.g., alkyl, m, n, etc., when it occurs more than once in any structure, is intended to be independent of its definition elsewhere in the same structure.

As used herein, the term “conjugate” refers to a molecule including a Fatty Acid component, optionally a linker component, and a biologically active (i.e. drug) component.

As used herein, the term “Fatty Acid” refers to a mono-, di-, or poly-carboxylic acid having one or more long aliphatic chains which are independently saturated, monounsaturated, or polyunsaturated. Preferably the Fatty Acid is a di, tri- or tetra-carboxylic acid (including functionalized derivatives thereof such as an amide bond attaching the carboxylic acid to the solubilizing portion of the Fatty Acid). The carboxylic acid group(s) are independently optionally phosphorylated, hydroxylated or sulfolated. However, preferably the carboxylic acid groups are not phosphorylated. Preferably there are two long aliphatic chains in the Fatty Acids of the disclosure. Preferably, the long aliphatic chain(s) is/are unsaturated. Preferably the long aliphatic chain(s) each independently contain(s) from 4 to 20 carbon atoms, for example from 6 to 18 carbon atoms, for example from 8 to 16 carbon atoms, for example 10 to 15 carbon atoms. The long aliphatic chain(s) may independently contain one or more —OH substituents, which, if present, are preferably at the end of the long aliphatic chain distal to the carboxylic acid group(s).

The term “linker” as used herein refers to a chemical moiety which joins the bifunctional protein degrader to the Fatty Acid in a conjugate of Formula (I), (I′) and subformulas thereof. Preferably the linker is a long, substantially straight-chained group including from 6 to 200, for example from 10 to 100, for example from 15 to 80, for example from 20 to 60 non-hydrogen atoms (typically selected from C, N, O and S, most typically selected from C, N and O). By “substantially straight-chained” it is meant that the main chain may be substituted by one or more groups each independently containing from 1 to 6 non-hydrogen atoms, preferably 1 to 4 non-hydrogen atoms (typically selected from C, N, O and S, most typically selected from C, N, and O). Such substituents may include, purely as an example, ═O, —C₁₋₄alkyl, —C₁₋₃hydroxyalkyl, —C₁₋₃aminoalkyl, —OH, —O—C₁₋₃alkyl, —NH₂, —N(H)—C₁₋₃alkyl, —N—(C₁₋₂alkyl)₂, —C₁₋₂alkylene-O—C₁₋₂alkyl, —C₁₋₂alkylene-N(H)—C₁₋₂alkyl, and —CH₂—N(C₁alkyl)₂.

As used herein, the term “C₁₋₆hydroxyalkyl” refers to a C₁₋₆alkyl radical as defined herein, wherein one of the hydrogen atoms of the C₁₋₆alkyl radical is replaced by OH. Examples of C₁₋₆ hydroxyalkyl include, but are not limited to, hydroxy-methyl, 2-hydroxy-ethyl, 2-hydroxy-propyl, 3-hydroxy-propyl and 5-hydroxy-pentyl.

As used herein, the term “C₁₋₆aminoalkyl” refers to a C₁₋₆alkyl radical as defined herein, wherein one of the hydrogen atoms of the C₁₋₆alkyl group is replaced by a primary amino group. Representative examples of C₁₋₆aminoalkyl include, but are not limited to, amino-methyl, 2-aminoethyl, 2-amino-propyl, 3-amino-propyl, 3-amino-pentyl and 5-amino-pentyl.

As used herein, the term “cleavable linker” or “cleavable portion” (of the linker) refers to a portion of the linker that is cleavable under conditions within the body. For instance, the cleavable portion of the linker can include a bond (e.g. an amide bond) which is susceptible to hydrolysis in the body.

As used herein, the term “solubilizing portion” or “solubilizing domain” (of the linker) refers to a portion of the linker that increases the solubility of the compound in vivo or in simulated gastrointestinal fluid. Preferably the solubilizing portion of the linker is, or comprises, polyethylene glycol, as defined herein. Alternatively, the solubilizing portion of the linker could comprise one or more hydrophilic substituents such as —F, —Cl, —OH, —CO₂H, —NH₂, ═O and the like in order to enhance solubility, for example as optional substituents to the C₅-C₁₀₀ alkylene, C₅-C₁₀₀ heteroalkylene, C₅-C₁₀₀ haloalkylene or polyethylene glycol.

As used herein, the term “solvate” refers to a complex of variable stoichiometry formed by a solute, for example, a conjugate of Formula (I), (I′) and subformulas thereof, and solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, such solvents selected for the purpose of the disclosure do not interfere with the biological activity of the solute. Solvates encompasses both solution-phase and isolatable solvates. Representative solvates include hydrates, ethanolates, methanolates, and the like.

As used herein, the term “hydrate” refers to a solvate wherein the solvent molecule(s) is/are water.

Various embodiments of the disclosure are described herein. It will be recognized that features specified in each embodiment may be combined with other specified features, including as indicated in the embodiments below, to provide further embodiments of the present disclosure.

It is understood that in the following embodiments, combinations of substituents or variables of the depicted formulae are permissible only if such combinations result in stable compounds.

Definitions of specific functional groups and chemical terms are described in more detail above and below. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} ed, Cambridge University Press, Cambridge, 1987.

Depending on the choice of the starting materials and procedures, the conjugates can be present in the form of one of the possible stereoisomers, rotamers, atropisomers, tautomers, or as mixtures thereof, for example, as subtantially pure optical isomers (antipodes), geometiric (cis or trans) stereoisomers, diastereoisomers, racemates or mixtures thereof, depending on the number of asymmetric carbon atoms. The present disclosure is meant to include all such possible stereoisomers, including racemic mixtures, diasteriomeric mixtures and optically pure forms. Optically active (R)- and (S)-tereoisomers may be prepared using chiral synthons or chiral reagents, or resolved using conventional techniques. If the conjugate contains a double bond, the substituent may be E or Z configuration. If the conjugate contains a disubstituted cycloalkyl, the cycloalkyl substituent may have a cis- or trans-configuration. All tautomeric forms are also intended to be included.

Certain compounds described herein may exist in particular geometric or stereoisomeric forms. If, for instance, a particular enantiomer of a compound described herein is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C or ¹⁴C enriched carbon are within the scope of this disclosure. Such compounds are useful, for example, as analytical tools, as probes in biological assays, or as therapeutic agents in accordance with the disclosure.

The conjugate may be present as an R-enantiomeric enriched mixture or an S-enantiomeric enriched mixture. In one embodiment, the conjugate is present as an R-enantiomeric enriched mixture. In another embodiment, the conjugate is present as an S-enantiomeric enriched mixture.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer. ee=(90−10)/100×100=80%.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words such compounds or compositions contain an enantiomeric excess of the S enantiomer over the R enantiomer.

Where a particular enantiomer is preferred, it may, in some embodiments be provided substantially free of the corresponding enantiomer, and may also be referred to as “optically enriched.” “Optically enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See e.g., Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw Hill, N Y, 1962); Wilen, S. H. Tables of Resolving Agents and Optical Resolutions p. 268 (E. L. Eliel, Ed., Univ. of Notre Dame Press, Notre Dame, IN 1972).

All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

### Pharmaceutically Acceptable Salts

Pharmaceutically acceptable salts of the compounds described herein are also contemplated for the uses described herein. As used herein, the terms “salt” or “salts” refer to an acid addition or base addition salt of a compound described herein. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein are capable of forming acid and/or base salts by virtue of the presence of amino and/or carboxyl groups or groups similar thereto. When both a basic group and an acid group are present in the same molecule, the compounds of the present invention may also form internal salts, e.g., zwitterionic molecules.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholanate, diethanolamine, diethylamine, lysine, meglumine, piperazine, and tromethamine.

Another embodiment is a compound of Formula (I), (I′) or subformulas thereof as an acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate, or xinafoate salt form.

### Pharmaceutical Compositions

Another embodiment is a pharmaceutical composition comprising one or more compounds of Formula (I), (I′) or subformulas thereof, or a pharmaceutically acceptable salt, hydrate, solvate, prodrug, stereoisomer, or tautomer thereof, and one or more pharmaceutically acceptable carrier(s). In an embodiment, the pharmaceutical composition is in a form suitable for oral or parenteral administration.

As used herein, the term “pharmaceutically acceptable carrier” refers to a substance useful in the preparation or use of a pharmaceutical composition or a pharmaceutically-acceptable material, composition or vehicle, and includes, for example, suitable liquid or solid fillers, diluents, solvents, encapsulating materials, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070). Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions of the disclosure are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween®, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The amount of the compounds of the present disclosure that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

The terms “pharmaceutically effective amount” or “therapeutically effective amount” means an amount of a conjugate according to the disclosure which, when administered to a patient in need thereof, is sufficient to effect treatment for disease-states, conditions, or disorders for which the conjugates have utility. Such an amount would be sufficient to elicit the biological or medical response of a tissue, system, or patient that is sought by a researcher or clinician. The amount of a conjugate of according to the disclosure which constitutes a therapeutically effective amount will vary depending on such factors as the conjugate and its biological activity, the composition used for administration, the time of administration, the route of administration, the rate of excretion of the conjugate, the duration of treatment, the type of disease-state or disorder being treated and its severity, drugs used in combination with or coincidentally with the conjugates of the disclosure, and the age, body weight, general health, sex, and diet of the patient. Such a therapeutically effective amount can be determined routinely by one of ordinary skill in the art having regard to their own knowledge, the prior art, and this disclosure.

### Isotopically Labelled Compounds

A conjugate described herein, or a pharmaceutically acceptable salt, hydrate, solvate, prodrug, stereoisomer, or tautomer thereof, is also intended to represent unlabeled forms as well as isotopically labeled forms of the conjugates. Isotopically labeled conjugates have structures depicted by the formulas given herein (e.g., compounds of Formula (I), (I′) and subformulas thereof) except that one or more atoms are replaced by an atom having a selected atomic mass or mass number. Isotopes that can be incorporated into conjugates of the disclosure include, for example, isotopes of hydrogen.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index or tolerability. It is understood that deuterium in this context is regarded as a substituent of a conjugate described herein, or a pharmaceutically acceptable salt, hydrate, solvate, prodrug, stereoisomer, or tautomer thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound described herein is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds described herein include isotopes of hydrogen, carbon, nitrogen, oxygen, phosphorous, fluorine, and chlorine, such as ³H, ¹¹C, ¹³C, ¹⁴C ¹⁵N, ¹⁸F, ³¹P ³²P, ³⁵S, ³⁶Cl, ¹²³I, ¹²⁴I, ¹²⁵I, respectively. The disclosure includes various isotopically labeled compounds as defined herein, for example, those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds described herein or a pharmaceutically acceptable salt, hydrate, solvate, prodrug, stereoisomer, or tautomer thereof, can generally be prepared by conventional techniques known to those skilled in the art or by processes analogous to those described in the accompanying Examples and Preparations using an appropriate isotopically-labeled reagents in place of the non-labeled reagent previously employed.

### Dosages

The pharmaceutical composition or combination of the present disclosure may, for example, be in unit dosage of about 1-1000 mg of active ingredient(s) for a subject of about 50-70 kg.

Toxicity and therapeutic efficacy of compounds described herein, including pharmaceutically acceptable salts and deuterated variants, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and thereby reduce side effects.

Data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### Methods of Use

The conjugates described herein in free form or in a pharmaceutically acceptable salt form, exhibit valuable pharmacological properties, e.g., modulating a Target Protein, e.g., as indicated in in vitro and in vivo tests as provided herein, and are therefore indicated for therapy or for use as research chemicals, e.g., as toll compounds.

Another embodiment is a method of modulating a Target Protein, e.g., a Target Protein listed in Table 1 or Table 2, in a subject in need thereof, the method comprising administering to the subject a therapeutically effective amount of a compound of Formula (I), (I′) and subformulas thereof, or a pharmaceutically acceptable salt, hydrate, solvate, prodrug, stereoisomer, or tautomer thereof.

Another embodiment is a method of inhibiting a Target Protein, e.g., a Target Protein listed in Table 1 or Table 2, in a subject in need thereof, the method comprising administering to the subject a therapeutically effective amount of a compound of Formula (I), (I′) and subformulas thereof, or a pharmaceutically acceptable salt, hydrate, solvate, prodrug, stereoisomer, or tautomer thereof.

Another embodiment is a method for inducing degradation of a Target Protein, e.g., a Target Protein listed in Table 1 or Table 2, in a subject in need thereof, the method comprising administering to the subject a therapeutically effective amount of a compound of Formula (I), (I) and subformulas thereof, or a pharmaceutically acceptable salt, hydrate, solvate, prodrug, stereoisomer, or tautomer thereof.

In an embodiment, any of the compounds of Formula (I), (I) and subformulas thereof disclosed herein, a pharmaceutically acceptable salt, hydrate, solvate, prodrug, stereoisomer, or tautomer thereof, are useful for the treatment or prevention of diseases and disorders associated with modulation of BTK protein levels through the binding to and altering of the specificity of a cereblon complex to induce proteasome-mediated degradation of BTK.

In another aspect, the disclosure provides a method of inhibiting, reducing, or eliminating the activity of a Target Protein, e.g., a Target Protein listed in Table 1 or Table 2, the method comprising administering to the subject a compound of Formula (I), (I) and subformulas thereof, or a pharmaceutically acceptable salt, hydrate, solvate, prodrug, stereoisomer, or tautomer thereof.

In an embodiment, is a method of degrading BTK in a subject, wherein the method comprises administering to the subject a therapeutically effective amount of a compound of Formula (I), (I) and subformulas thereof, or pharmaceutically acceptable salt, amide, ester, solvate, stereoisomer, or tautomer thereof.

In an embodiment, inhibiting, reducing, or eliminating the activity of a Target Protein, e.g., a Target Protein listed in Table 1 or Table 2, comprises recruiting a ligase (e.g., Cereblon E3 Ubiquitin ligase) with the Targeting Ligase Binder, e.g., a Targeting Ligase Binder described herein, of the bifunctional protein degrader, e.g., a bifunctional protein degrader described herein, forming a ternary complex of the Target Protein, the fatty acid-bifunctional degrader conjugate, e.g., a compound of Formula (I), (I) and subformulas thereof, and the ligase, to thereby inhibit, reduce or eliminate the activity of the Target Protein.

In an embodiment, the disclosure provides a method of treating a target protein-mediated disorder, disease, or condition in a patient comprising administering to the patient any of the compounds of Formula (I), (I) and subformulas thereof described herein, or a pharmaceutically acceptable salt, hydrate, solvate, prodrug, stereoisomer, or tautomer thereof.

Another embodiment is a method for treating or preventing a cancer mediated by a Target Protein, e.g., a Target Protein listed in Table 1 or Table 2, in a subject in need thereof comprising administering a compound of Formula (I), (I) and subformulas thereof, or a pharmaceutically acceptable salt, hydrate, solvate, prodrug, stereoisomer, or tautomer thereof to the subject.

Another embodiment is a method of treating or preventing a disease mediated by BTK in a subject in need thereof, the method comprising administering to the subject a therapeutically effective amount of any of the compounds of Formula (I), (I′) and subformulas thereof described herein, or a pharmaceutically acceptable salt, hydrate, solvate, prodrug, stereoisomer, or tautomer thereof.

Another embodiment is a method of treating or preventing a respiratory disorder, a proliferative disorder, an autoimmune disorder, an autoinflammatory disorder, an inflammatory disorder, a neurological disorder, and an infectious disease or disorder mediated by a Target Protein, e.g., a Target Protein listed in Table 1 or Table 2, in a subject in need thereof, the method comprising administering to the subject a therapeutically effective amount of a compound of Formula (I), (I′) and subformulas thereof, or a pharmaceutically acceptable salt, hydrate, solvate, prodrug, stereoisomer, or tautomer thereof. In an embodiment, the disorder is a proliferative disorder. In an embodiment, the proliferative disorder is cancer.

Another embodiment is a method of treating or preventing a cancer in a subject in need thereof, the method comprising administering to the subject a therapeutically effective amount of a compound of Formula (I), (I′) and subformulas thereof, or a pharmaceutically acceptable salt, hydrate, solvate, prodrug, stereoisomer, or tautomer thereof.

In an embodiment, the cancer is a neoplastic disease and includes, for instance, solid tumors such as e.g. sarcomas or carcinomas or blood cancer such as e.g. leukemia or myeloma, or cancers of lymphatic system such as lymphoma, or mixed types thereof. In an embodiment, the cancer is selected from the group consisting of chronic lymphocytic leukemia (CLL), mantle cell lymphoma (MCL), small lymphocytic lymphoma (SLL), Waldenstrom's macroglobulinemia, diffuse large B-cell lymphoma (DLBCL), follicular lymphoma, Burkitt lymphoma, Marginal Zone Lymphoma, immunoblastic large cell lymphoma, Richter Syndrome, and precursor B-lymphoblastic lymphoma, primary and secondary multiple myeloma, B-cell prolymphocytic leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, lymphomatoid granulomatosis, and acute lymphoblastic leukemia, Hodgkin lymphoma, non-Hodgkin lymphoma, post-transplant lymphoproliferative disorder, hairy cell leukemia, and Histiocytic and dendritic neoplasms.

In another aspect, the disclosure provides compounds Formula (I), (I′) and subformulas thereof, or a pharmaceutically acceptable salt, hydrate, solvate, prodrug, stereoisomer, or tautomer thereof, for use in inhibiting or modulating a target protein in a subject in need thereof.

In another aspect, the disclosure provides compounds of Formula (I), (I′) and subformulas thereof, or a pharmaceutically acceptable salt, hydrate, solvate, prodrug, stereoisomer, or tautomer thereof, for use in inhibiting a target protein in a subject in need thereof.

In another aspect, the disclosure provides compounds of Formula (I), (I′) and subformulas thereof, or a pharmaceutically acceptable salt, hydrate, solvate, prodrug, stereoisomer, or tautomer thereof, for use to treat or to prevent diseases and disorders associated with reducing or decreasing BTK protein levels through the binding to and altering of the specificity of a cereblon complex to induce proteasome-mediated degradation of BTK.

Another embodiment is a pharmaceutical composition comprising a compound of Formula (I), (I′) and subformulas thereof, or a pharmaceutically acceptable salt, hydrate, solvate, prodrug, stereoisomer, or tautomer thereof, and a pharmaceutically acceptable carrier, for use in inhibiting a Target Protein, e.g., a Target Protein listed in Table 1 or Table 2, in a subject in need thereof.

Another embodiment is compounds of Formula (I), (I′) and subformulas thereof, or a pharmaceutically acceptable salt, hydrate, solvate, prodrug, stereoisomer, or tautomer thereof, for use in treating or preventing a respiratory disorder, a proliferative disorder, an autoimmune disorder, an autoinflammatory disorder, an inflammatory disorder, a neurological disorder, and an infectious disease or disorder mediated by a Target Protein, e.g., a Target Protein listed in Table 1 or Table 2, in a subject in need thereof. In an embodiment, the use is for treating or preventing a proliferative disorder.

Another embodiment is compounds of Formula (I), (I′) and subformulas thereof, or a pharmaceutically acceptable salt, hydrate, solvate, prodrug, stereoisomer, or tautomer thereof, for use in treating or preventing a cancer in a subject in need thereof.

In an embodiment, the cancer is a neoplastic disease and includes, for instance, solid tumors such as e.g. sarcomas or carcinomas or blood cancer such as e.g. leukemia or myeloma, or cancers of lymphatic system such as lymphoma, or mixed types thereof. In an embodiment, the cancer is selected from the group consisting of chronic lymphocytic leukemia (CLL), mantle cell lymphoma (MCL), small lymphocytic lymphoma (SLL), Waldenstrom's macroglobulinemia, diffuse large B-cell lymphoma (DLBCL), follicular lymphoma, Burkitt lymphoma, Marginal Zone Lymphoma, immunoblastic large cell lymphoma, Richter Syndrome, and precursor B-lymphoblastic lymphoma, primary and secondary multiple myeloma, B-cell prolymphocytic leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, lymphomatoid granulomatosis, and acute lymphoblastic leukemia, Hodgkin lymphoma, non-Hodgkin lymphoma, post-transplant lymphoproliferative disorder, hairy cell leukemia, and Histiocytic and dendritic neoplasms.

Another embodiment is the use of a compound of Formula (I), (I′) and subformulas thereof, or a pharmaceutically acceptable salt, hydrate, solvate, prodrug, stereoisomer, or tautomer thereof, in the manufacture of a medicament for inhibiting or modulating a Target Protein, e.g., a Target Protein listed in Table 1 or Table 2, in a subject in need thereof.

Another embodiment is a use of a compound of Formula (I), (I′) and subformulas thereof, or a pharmaceutically acceptable salt, hydrate, solvate, prodrug, stereoisomer, or tautomer thereof, in the manufacture of a medicament for inhibiting a Target Protein, e.g., a Target Protein listed in Table 1 or Table 2, in a subject in need thereof.

Another embodiment is a use of a compound of Formula (I), (I′) and subformulas thereof, or a pharmaceutically acceptable salt, hydrate, solvate, prodrug, stereoisomer, or tautomer thereof, in the manufacture of a medicament for treating a diesease mediated by BTK.

Another embodiment is a use of a compound of Formula (I), (I′) and subformulas thereof, or a pharmaceutically acceptable salt, hydrate, solvate, prodrug, stereoisomer, or tautomer thereof, in the manufacture of a medicament for treating or preventing a cancer mediated by a Target Protein, e.g., a Target Protein listed in Table 1 or Table 2, in a subject in need thereof.

In an embodiment, the cancer is a neoplastic disease and includes, for instance, solid tumors such as e.g. sarcomas or carcinomas or blood cancer such as e.g. leukemia or myeloma, or cancers of lymphatic system such as lymphoma, or mixed types thereof. In an embodiment, the cancer is selected from the group consisting of chronic lymphocytic leukemia (CLL), mantle cell lymphoma (MCL), small lymphocytic lymphoma (SLL), Waldenstrom's macroglobulinemia, diffuse large B-cell lymphoma (DLBCL), follicular lymphoma, Burkitt lymphoma, Marginal Zone Lymphoma, immunoblastic large cell lymphoma, Richter Syndrome, and precursor B-lymphoblastic lymphoma, primary and secondary multiple myeloma, B-cell prolymphocytic leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, lymphomatoid granulomatosis, and acute lymphoblastic leukemia, Hodgkin lymphoma, non-Hodgkin lymphoma, post-transplant lymphoproliferative disorder, hairy cell leukemia, and Histiocytic and dendritic neoplasms.

Another embodiment is a use of a compound of Formula (I), (I′) and subformulas thereof, or a pharmaceutically acceptable salt, hydrate, solvate, prodrug, stereoisomer, or tautomer thereof, in the manufacture of a medicament for treating or preventing a respiratory disorder, a proliferative disorder, an autoimmune disorder, an autoinflammatory disorder, an inflammatory disorder, a neurological disorder, and an infectious disease or disorder in a subject in need thereof.

### Combination Therapy

The conjugate of the present disclosure may be administered either simultaneously with, or before or after, one or more other therapeutic agent. The conjugate of the present disclosure may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a conjugate of the present disclosure.

In one embodiment, the disclosure provides a product comprising a conjugate of the present disclosure and at least one other therapeutic agent as a combined preparation for simultaneous, separate or sequential use in therapy. In one embodiment, the therapy is treatment of a disease or condition mediated by a Target Protein. Products provided as a combined preparation include a composition comprising the conjugate of the present disclosure and the other therapeutic agent(s) together in the same pharmaceutical composition, or the conjugate of the present disclosure and the other therapeutic agent(s) in separate form, e.g., in the form of a kit.

In one embodiment, the disclosure provides a pharmaceutical composition comprising a conjugate of the present disclosure and another therapeutic agent(s). Optionally, the pharmaceutical composition may comprise a pharmaceutically acceptable carrier, as described above.

In one embodiment, the disclosure provides a kit comprising two or more separate pharmaceutical compositions, at least one of which contains a conjugate of the present disclosure. In one embodiment, the kit comprises means for separately retaining said compositions, such as a container, divided bottle, or divided foil packet. An example of such a kit is a blister pack, as typically used for the packaging of tablets, capsules and the like.

The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

Combination therapy includes the administration of the conjugates disclosed herein in further combination with other biologically active ingredients (such as, but not limited to, a second and different anticancer agent, an antiproliferative agent, etc.) and non-drug therapies (such as, but not limited to, surgery or radiation treatment). For instance, the conjugates of the application can be used in combination with other pharmaceutically active compounds, preferably compounds that are able to enhance the effect of the conjugates of the application. The conjugates of the application can be administered simultaneously (as a single preparation or separate preparation) or sequentially to the other drug therapy or treatment modality. In general, a combination therapy envisions administration of two or more drugs during a single cycle or course of therapy.

Another embodiment is a pharmaceutical combination comprising a compound of Formula (I), (I′) and subformulas thereof, or a pharmaceutically acceptable salt, hydrate, solvate, prodrug, stereoisomer, or tautomer thereof, and one or more additional therapeutic agent(s) for simultaneous, separate or sequential use in therapy.

In an embodiment, the additional therapeutic agent is selected from the group consisting of: an antiproliferative agent, anticancer agent, immunomodulatory agent, an anti-inflammatory agent, a neurological treatment agent, an anti-viral agent, an anti-fungal agent, anti-parasitic agent, an antibiotic, and a general anti-infective agent.

In an embodiment, the additional therapeutic agent is a second a target protein inhibitor.

In another embodiment, the additional therapeutic agent is selected from the group consisting of: a second a kinase inhibitor, kinase modulator and kinase degrader.

In the combination therapies of the disclosure, the conjugate of the present disclosure and the other therapeutic agent may be manufactured and/or formulated by the same or different manufacturers. Moreover, the conjugate of the present disclosure and the other therapeutic may be brought together into a combination therapy: (i) prior to release of the combination product to physicians (e.g. in the case of a kit comprising the conjugate of the present disclosure and the other therapeutic agent); (ii) by the physician themselves (or under the guidance of the physician) shortly before administration; (iii) in the patient themselves, e.g. during sequential administration of the conjugate of the present disclosure and the other therapeutic agent.

Accordingly, the disclosure provides the use of a conjugate of the present disclosure for treating a disease or condition mediated by Bruton's tyrosine kinase, wherein the medicament is prepared for administration with another therapeutic agent. The disclosure also provides the use of another therapeutic agent for treating a disease or condition mediated by Bruton's tyrosine kinase, wherein the medicament is administered with a conjugate of the present disclosure.

The disclosure also provides a conjugate of the present disclosure for use in a method of treating a disease or condition mediated by Bruton's tyrosine kinase, wherein the conjugate of the present disclosure is prepared for administration with another therapeutic agent. The disclosure also provides another therapeutic agent for use in a method of treating a disease or condition mediated by Bruton's tyrosine kinase, wherein the other therapeutic agent is prepared for administration with a conjugate of the present disclosure. The disclosure also provides a conjugate of the present disclosure for use in a method of treating a disease or condition mediated by Bruton's tyrosine kinase, wherein the conjugate of the present disclosure is administered with another therapeutic agent. The disclosure also provides another therapeutic agent for use in a method of treating a disease or condition mediated by Bruton's tyrosine kinase, wherein the other therapeutic agent is administered with a conjugate of the present disclosure.

The disclosure also provides the use of a conjugate of the present disclosure for treating a disease or condition mediated by Bruton's tyrosine kinase, wherein the patient has previously (e.g. within 24 hours) been treated with another therapeutic agent. The disclosure also provides the use of another therapeutic agent for treating a disease or condition mediated by Bruton's tyrosine kinase, wherein the patient has previously (e.g. within 24 hours) been treated with a conjugate of the present disclosure.

In one embodiment, the other therapeutic agent for use in combination therapy is selected from:

Apoptosis modulators, Anti-CD20 antibodies, Anti-CD22 antibodies, PI3K inhibitors, Tyrosine kinase inhibitors, Immune checkpoint agents, CART therapeutic agents, Immunomodulators, bispecific antibodies targeting CD20 and CD3, antibody-drug conjugates (ADC), Proteasome inhibitors, epigenetic modifiers, Anti-CD38 mAb, Anti-SLAMF7 agent, XPO1 inhibitors and other agents such as chemotherapeutic agents.

In an embodiment the apoptosis modulators are selected from Bcl2 inhibitors (such as Antimycin, obatoclax, venetoclax (Venclexta®), ethyl-2-amino-6-cyclopentyl-4-(1-cyano-2-ethoxy-2-oxoethyl)-4H-chromone-3-carboxylate (HA14-1), oblimersen (G3139, Genasense®), Bak BH3 peptide, (−)-Gossypol (AT-101, BL-193), Navitoclax (ABT-263)), Mcl1 inhibitors (such as AMG176, S63845, AZD5991, MIK665), and MDM2/p53 inhibitors (such as NVP-HDM201, NVP-CGM-097, ALRN-6924, idasanutlin, AMG232, and DS-3032B).

In an embodiment the Anti-CD20 antibodies are selected from Rituximab, obinutuzumab, ofatumumab, ocrelizumab, and ublituximab.

In an embodiment the Anti-CD22 antibodies are selected from Inotuzumab, epratuzumab, bectumomab, and moxetumomab.

In an embodiment the PI3K inhibitors are selected from duvelisib, umbralisib tosylate, INCB050465, apilimod mesylate (LAM-002), copanlisib hydrochloride (Aliqopa®), tenalisib, pictilisib (GDC 0941), sonolisib (PX866), pilaralisib (SAR 245408 or XL 147), alpelisib (BYL719), and leniolisib (CDZ173).

In an embodiment the Tyrosine kinase inhibitors are selected from BTK inhibitors (such as ibrutinib, acalabrutinib, zanubrutinib (BGB-3111), tirabrutinib (ONO-4059), ARQ531, CC-292 (AVL-292), CT-1530, DTRMWXHS-12, GDC-0853, M7583, and vecabrutinib (SNS-062), SYK inhibitors (such as entospletinib (GS9973), fostamatinib, and HMPL-523, the SYK/JAK inhibitor cerdulatinib (PRT062070), SYK/FLT inhibitors such as TAK-659, FLT3 inhibitors such as FF-10101, the FLT3/BTK inhibitor (CG806), JAK inhibitors (such as itacitanib, INCB052793, BMS911543, fedratinib, WP-1066, NS-018, and ruxolitinib (Jakavi®)), Erlotinib hydrochloride (Tarceva®), Linifanib (ABT869), Sunitinib malate (Sutent®), Bosutinib (Bosulif®), Dasatinib (Sprycel®), Pazopanib (Votrient®), Sorafenib (Nexavar®), Zactima (ZD6474), Imatinib or Imatinib mesylate (Gilvec® and Gleevec®), and tozasertib (VX680 or MK-0457).

In an embodiment the Immune checkpoint agent is an Anti-PD-1 agent, anti-PD-L1 agent selected from Pembrolizumab, nivolumab, tislelizumab, atezolizumab, ipilimumab, cemiplimab, TLR4 agonist, CCR4 mAb mogamulizumab and CD47 mAb fusion protein (TTI-621).

In an embodiment the CART therapy is selected from CD19, BCMA CART, CD20, CD79b, CD22, CD30.

In an embodiment the immunomodulators are selected from lenalidomide (Revlimid®), thalidomide (Thalomid®), avadomide (CC-122), and pomalidomide (Actimid®, Imnovid®, Pomalyst®).

In an embodiment the bispecific antibody targeting CD20 and CD3 is selected from REGN-1979, XmAb-13676, BTCT-4465-A, CD20-TCB, and 8RG-6026.

In an embodiment the ADC is selected from CD79 ADC polatuzumab vedotin, CD30 ADC brentuximab vedotin, CD25 ADC camidanlumab tesirine, and CD19 ADC loncastuximab tesirine.

In an embodiment the proteasome inhibitors are selected from Bortezomib (Velcade®), carfilzomib (Kyprolis®), marizomib (NPI-0052), ixazomib citrate (MLN-9708, Ninlaro®), delanzomib (CEP-18770), and oprozomib (ONX-0912).

In an embodiment the epigenetic modifiers such as HDAC and DNA methylation inhibitors are selected from Vorinostat (Zolinza®), Romidepsin (Istodax®), azacitidine (Mylosar®, Vidaza®), Pyroxamide, Spiruchostatin A, Mylproin (Valproic acid), Entinostat, and guadecitabine.

In an embodiment the Anti-CD38 mAb is selected from Daratumumab and Isatuximab.

In an embodiment the Anti-SLAMF7 agent is Elotuzumab.

In an embodiment the XPO1 inhibitors are selected from Selinexor and Eltanexor.

In an embodiment other agents, such as general chemotherapeutic agents, which may be combined with a compound of the disclosure are selected from anastrozole (Arimidex®), bendamustine (Treanda®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), epirubicin (Ellence®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), ROR mAb cirmtuzumab, Dual PI3K/HDAC inhibitor (CUDC-907), Bet inhibitors (INCB357643), ALK inhibitors (crizotinib), EZH1/2 inhibitors (DS-3201b), MAPK inhibitors, Aplidin, Plitidepsin (eEF1A2 inhibitor), Wnt inhibitors, radiopharmaceuticals, idiotype vaccines, Pegfilgrastim (Neulasta®), citoplurikin (IRX-2).

In a further embodiment, the other therapeutic agent is selected from:

venetoclax, oblimersen, navitoclax, MIK665, NVP-HDM201, Rituximab, obinutuzumab, ofatumumab, ocrelizumab, ublituximab, Inotuzumab, epratuzumab, bectumomab, moxetumomab, duvelisib, umbralisib tosylate, INCB050465, leniolisib (CDZ173), apilimod mesylate (LAM-002), copanlisib hydrochloride, tenalisib, pictilisib, alpelisib, ibrutinib, acalabrutinib, zanubrutinib (BGB-3111), tirabrutinib (ONO-4059), ARQ531, CC-292 (AVL-292), CT-1530, DTRMWXHS-12, GDC-0853, M7583, vecabrutinib (SNS-062), entospletinib, (GS9973), fostamatinib, HMPL-523, cerdulatinib (PRT062070), (TAK-659), FF-10101, FLT3/BTK inhibitor (CG806), itacitanib, INCB052793, BMS911543, fedratinib, WP-1066, NS-018, ruxolitinib (Jakavi®), Pembrolizumab, nivolumab, tislelizumab, atezolizumab, ipilimumab, cemiplimab, TLR4 agonist, CCR4 mAb mogamulizumab, CD47 mAb fusion protein (TTI-621), CD19, BCMA CART, CD20, CD79b, CD22, CD30, lenalidomide, thalidomide, avadomide, pomalidomide, XmAb-13676, CD79 ADC polatuzumab vedotin, CD30 ADC brentuximab vedotin, CD25 ADC camidanlumab tesirine, CD19 ADC loncastuximab tesirine, Carfilzomib, Bortezomib, Ixazomib, marizomib, oprozomib, Azacitidine, Romidepsin, Vorinostat, guadecitabine, Daratumumab, Isatuximab, Elotuzumab, Selinexor, Eltanexor, Fludarabine, carmustine, cyclophosphamide, chlorambucil, bendamustine, melphalan, cladribine, dacarbazine, pentostatin, vincristine, etoposide, epirubicin, doxorubicin, anthracyclines and antifolate agents.

In a further embodiment, the other therapeutic agent is selected from a Bcl2 inhibitor and a BTK inhibitor.

In a further embodiment, the other therapeutic agent is selected from venetoclax, ibrutinib, and acalabrutinib.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3rd edition, John Wiley & Sons). These groups are removed at a convenient stage of the compound synthesis using methods that are readily apparent to those skilled in the art.

### Analytical Methods, Materials, and Instrumentation

Unless otherwise noted, reagents and solvents were used as received from commercial suppliers. Proton nuclear magnetic resonance (H-NMR) spectra were acquired on Bruker AVANCE 400 MHz, 500 MHz or 600 MHz NMR spectrometers using ICON-NMR, under TopSpin program control unless otherwise noted. Spectra were measured at 298K, unless indicated otherwise, and were referenced relative to the solvent resonance. Tetramethylsilane (TMS) was used as an internal standard. Chemical shifts are reported in ppm relative to dimethyl sulfoxide (δ 2.50), methanol (δ 3.31), chloroform (δ 7.26) or other solvent as indicated in NMR spectral data. A small amount of the dry sample (2-5 mg) is dissolved in an appropriate deuterated solvent (1 mL). The chemical names were generated using ChemBioDraw Ultra v17 from CambridgeSoft.

Mass spectra were acquired on LC-MS, SFC-MS, or GC-MS systems using electrospray, chemical and electron impact ionization methods from a range of instruments of the following configurations:
Waters Acquity UPLC/SQD system, using a photodiode array detector and a single quadrupole mass detector
Agilent 1200 systems with G 6110 series mass detector
Agilent 1290 Infinity II with DAD (photodiode array detector) and single quadrupole mass detector with ESI and APCI ionization (multi-mode).
Waters AcQuity UPLC with PDA (photodiode array detector), ELSD and single quadrupole mass detector with ESI ionization
Waters AutoPurification System with PDA (photodiode array detector) and single quadrupole mass detector with ESI ionization.
   [M+H]⁺ refers to protonated molecular ion of the chemical species.
   [M−H]⁻ refers to molecular ion of the chemical species with loss of one proton.
   [M+Na]⁺ refers to molecular ion of the chemical species with addition of one sodium ion.
   [M−Boc+H]⁺ refers to protonated molecular ion of the chemical species without a Boc protecting group.
   [M−tBu+H]⁺ refers to protonated molecular ion of the chemical species without a tert-butyl group.
   [M−H−Pfp]⁻ refers to a molecular ion of the chemical species without a Pfp group and loss of one proton.
   [M+2H]²⁺ refers to a doubly protonated molecular ion of the chemical species.
   [M+NH₄+H]²⁺ refers to an ammonia adduct of [M+2H]²⁺.

### LCMS Method T1:

Column: Waters Acquity HSS T3 1.8 μm 2.1×50 mm or 2.1×100 mm
Column temperature: 60° C.
Eluents: A: aq. formic acid (0.05%)+aq. ammonium acetate (3.75 mM)
   B: ACN+formic acid (0.04%)
Flow rate: 1.0 mL/min
Gradient: 5% to 98% B in 1.4 min

### LCMS Method T2:

Column: Waters Acquity UPLC® HSS T3 1.8 μm 2.1×50 mm
Column temperature: 60° C.
Eluents: A: aq. formic acid (0.05%)+aq. ammonium acetate (3.75 mM)
   B: ACN+formic acid (0.04%)
Flow rate: 1.0 mL/min
Gradient: 5% to 98% B in 1.4 min
Detection UV/VIS (DAD), ELSD, ESI (+/−), 100-1200 m/z

### LCMS Method T3:

Column: XBridge C18 (4.6×50 mm, 3.5 μm)
Column temperature: 50° C.
Eluents: A: aq. ammonium hydrogen carbonate (10 mM)
   B: ACN
Flow rate: 1.8 mL/min
Gradient: 5% to 95% B in 1.5 min

### LCMS Method T4:

Column: SunFire C18 (4.6×50 mm, 3.5 μm)
Column temperature: 50° C.
Eluents: A: water+TFA (0.01%)
   B: ACN+TFA (0.01%)
Flow rate: 2.0 mL/min
Gradient: 5% to 95% B in 1.4 min

### LCMS Method T5:

Column: SunFire C18 (4.6×50 mm, 3.5 μm)
Column temperature: 50° C.
Eluents: A: water+TFA (0.01%)
   B: ACN+TFA (0.01%)
Flow rate: 2.0 mL/min
Gradient: 5% to 95% B in 1.3 min

### LCMS Method T6:

Column: HALO C18 (4.6×30 mm, 2.7 μm)
Column temperature: 50° C.
Eluents: A: water+TFA (0.01%)
   B: ACN+TFA (0.01%)
Flow rate: 2.2 mL/min
Gradient: 5% to 95% B in 1.0 min

### LCMS Method T7:

Column: ACQUITY UPLC BEH C18, 130 Å, 1.7 μm, 2.1 mm×30 mm
Column temperature: 50° C.
Eluents: A: water+formic acid (0.1%)
   B: ACN+formic acid (0.1%)
Flow rate: 1.0 mL/min
Gradient: 2% to 98% B in 2.0 min

### LCMS Method T8:

Column: ACQUITY UPLC BEH C18, 130 Å, 1.7 μm, 2.1 mm×50 mm
Column temperature: 50° C.
Eluents: A: water+formic acid (0.1%)
   B: ACN+formic acid (0.1%)
Flow rate: 1.0 mL/min
Gradient: 5% to 95% B in 5.2 min

### LCMS Method T9:

Column: ACQUITY UPLC BEH C18, 1.7 μm, 2.1 mm×30 mm
Column temperature: 50° C.
Eluents: A: water+ammonium hydroxide (5 mM)
   B: ACN+ammonium hydroxide (5 mM)
Flow rate: 1.0 mL/min
Gradient: 2% to 98% B in 2.0 min

### LCMS Method T10:

Column: Acquity BEH C18, 130 Å 1.7 μm 2.1×50 mm, 2.1 mm×50 mm
Column temperature: 50° C.
Eluents: A: water+ammonium hydroxide (5 mM)
   B: ACN+ammonium hydroxide (5 mM)
Flow rate: 1.0 mL/min
Gradient: 2% to 98% B in 5.2 min

### LCMS Method T11:

Column: Acquity BEH C18, 130 Å 1.7 μm 2.1×50 mm, 2.1 mm×50 mm
Column temperature: 50° C.
Eluents: A: water+0.1% formic acid
   B: ACN+0.1% formic acid
Flow rate: 1.0 mL/min
Gradient: 2% to 98% B in 1.0 min
   98% B to 1.30 min.

### LCMS Method T12:

Column: Acquity BEH C18, 130 Å 1.7 μm 2.1×50 mm, 2.1 mm×50 mm
Column temperature: 50° C.
Eluents: A: water+0.1% formic acid
   B: ACN+0.1% formic acid
Flow rate: 1.0 mL/min
Gradient: 40% to 98% B in 3.4 min
   98% B to 5.15 min.

### LCMS Method W1:

Column: Waters Acquity UPLC® BEH C18 1.7 μm 2.1×100 mm
Column temperature: 80° C.
Eluents: A: water+0.05% formic acid+aq. ammonium acetate (3.75 mM)
   B: IPA+0.05% formic acid
Flow rate: 0.4 mL/min
Gradient: 5% to 60% B in 8.4 min, 60 to 98% B in 1.0 min

### LCMS Method W2:

Column: XBridge C18, 4.6×50 mm, 3.5 μm
Column temperature: 40° C.
Eluents: A: aq. ammonium hydrogen carbonate (10 mM)
   B: ACN
Flow rate: 1.8 mL/min
Gradient: 5% to 95% B in 1.4 min, 95% B for 1.6 min

### LCMS Method W3:

Column: SunFire C18, 4.6×50 mm, 3.5 μm
Column temperature: 50° C.
Eluents: A: aq. TFA (0.01%)
   B: ACN containing TFA (0.01%)
Flow rate: 2.0 mL/min
Gradient: 5% to 95% B in 1.2 min, 95% B for 1.3 min

### LCMS Method W4:

Column: SunFire C18, 3×30 mm, 2.5 μm
Column temperature: 50° C.
Eluents: A: aq. TFA (0.01%)
   B: ACN containing TFA (0.01%)
Flow rate: 1.5 mL/min
Gradient: 5% to 95% B in 1.5 min

### LCMS Method W5:

Column: Waters ACQUITY UPLC® BEH C18 1.7 μm (2.1×50 mm)
Column temperature: 80° C.
Eluents: A: water+0.05% formic acid+aq. ammonium acetate (3.75 mM)
   B: IPA+0.05% formic acid
Flow rate: 0.6 mL/min
Gradient: 5% to 98% B in 1.7 min

### LCMS Method W6:

Column: Waters Acquity UPLC® HSS T3 1.8 μm 2.1×50 mm
Column temperature: 60° C.
Eluents: A: aq. formic acid (0.05%)+aq. ammonium acetate (3.75 mM)
   B: ACN containing formic acid (0.04%)
Flow rate: 1.0 mL/min
Gradient: 5% to 98% B in 1.4 min
Detection UV/VIS (DAD), ELSD, ESI (+/−), 800-2000 m/z

### LCMS Method W7:

Column: Waters Acquity UPLC® HSS T3 1.8 μm 2.1×100 mm
Column temperature: 60° C.
Eluents: A: aq. formic acid (0.05%)+aq. ammonium acetate (3.75 mM)
   B: ACN containing formic acid (0.04%)
Flow rate: 0.8 mL/min
Gradient: 5% to 98% B in 9.4 min
Detection UV/VIS (DAD), ELSD, ESI (+/−), 100-1200 m/z

### LCMS Method W8:

Column: Waters Acquity UPLC® BEH C18 1.7 μm 2.1×50 mm
Column temperature: 80° C.
Eluents: A: water+4.76% IPA+0.05% formic acid+aq. ammonium acetate (3.75 mM)
   B: IPA+0.05% formic acid
Flow rate: 0.6 mL/min
Gradient: 1% to 98% B in 1.7 min

### LCMS Method W9:

Column: Waters Acquity UPLC® BEH C18 1.7 μm 2.1×100 mm
Column temperature: 80° C.
Eluents: A: water+4.76% IPA+0.05% formic acid+aq. ammonium acetate (3.75 mM)
   B: IPA+0.05% formic acid
Flow rate: 0.4 mL/min
Gradient: 1% to 60% B in 8.4 min, 60 to 98% B in 1.0 min

### LCMS Method W10:

Column: ACQUITY UPLC BEH C18, 130 Å, 1.7 μm, 2.1 mm×30 mm
Column temperature: 50° C.
Eluents: A: water+TFA (0.05%)
   B: ACN+TFA (0.05%)
Flow rate: 1.0 mL/min
Gradient: 2% to 98% B in 2.0 min

### LCMS Method W11:

Column: Waters XBridge BEH C18, 2.5 μm, 3.0×75 mm
Column temperature: 60° C.
Eluents: A: 0.05% TFA in (water/ACN, 95/5, v/v)
   B: 0.05% TFA in (water/ACN, 5/95, v/v)
Flow rate: 1.0 mL/min
Gradient: 10% to 100% B in 23.0 min

### LCMS Method C1:

Column: Waters Acquity BEH C18, 1.7 μm, 2.1 mm×100 mm.
Column temperature: 40° C.
Eluents: A: 0.1% formic acid in water/ACN (95/5, v/v)
   B: 0.1% formic acid in ACN/water (95/5, v/v)
Flow rate: 0.5 mL/min
Gradient: 5% to 95% B in 9.0 min

### LCMS Method C2:

Column: ACQUITY UPLC BEH C18, 1.7 μm, 2.1 mm×50 mm
Eluents: A: water+trifluoroacetic acid (0.05%)
   B: ACN+trifluoroacetic acid (0.05%)
Flow rate: 0.5 mL/min
Gradient: 5% to 95% B in 5.0 min

### LCMS Method C3:

Column: Kinetex EVO C18, 5 μm, 2.1 mm×30 mm
Column temperature: 50° C.
Eluents: A: water+TFA (0.0375%)
   B: ACN+TFA (0.0187%)
Flow rate: 1.5 mL/min
Gradient: 5% to 95% B in 1.55 min

### LCMS Method C4:

Column: ACQUITY BEH Shield RP18, 1.7 um, 2.1×100 mm
Column temperature: 30° C.
Eluents: A: 10 mM ammonium acetate in 95% water/5% ACN
   B: 95% ACN/5% Water
Flow rate: 0.5 mL/min
Gradient: 20% to 95% B in 8.5 min

### LCMS Method C5:

Instrument: Waters UPC2 analytical SFC (SFC-H)
Column: ChiralPak AD, 150×4.6 mm I.D., 3 μm
Mobile phase: A: CO₂
   B: EtOH (0.05% DEA)
Gradient: B 25%
Flow rate: 2.5 mL/min
Back pressure: 100 bar
Column temperature: 35° C.
Wavelength: 220 nm

### LCMS Method C6:

Column: Waters XBridge BEH C18, 2.5 μm, 3.0×75 mm
Column temperature: 60° C.
Eluents: A: 0.05% TFA in (water/ACN, 95/5, v/v)
   B: 0.05% TFA in (water/ACN, 5/95, v/v)
Flow rate: 1.0 mL/min
Gradient: 10% to 100% B in 23.0 min

### Method C7:

Instrument: Shimadzu Prominence LC-20AT
Column: 250×4.6 mm, 5 μm Chiralpak IG
Mobile phase: MTBE:DCM:EtOH:ACN=35:35:25:5 (v:v:v:v)+0.15% DEA
Flow rate: 1 mL/min
Column temperature: 35° C.
Wavelength: 220 nm and 254 nm
Run Time (min): 25.00

### LCMS Method G1:

Column: Waters Acquity UPLC® BEH C18, 1.7 μm, 2.1×100 mm
Column temperature: 40° C.
Eluents: A: TFA (0.05%) in Water
   B: TFA (0.05%) in ACN
Flow rate: 0.3 mL/min
Gradient: Isocratic: 5:95 (A:B), Run Time: 8 min
Detection ELSD, ESI (+/−), 100-1200 m/z

### LCMS Method G2:

Column: Waters Acquity UPLC® BEH C18, 1.7 μm, 2.1×100 mm
Column temperature: 25° C.
Eluents: A: TFA (0.05%) in water
   B: TFA (0.05%) in ACN
Flow rate: 0.5 mL/min
Gradient: Isocratic: 5:95 (A:B), Run Time: 8 min
Detection ELSD, ESI (+/−), 100-1200 m/z

### LCMS Method G3:

Column: Waters Acquity UPLC® BEH C18, 1.7 μm, 2.1×100 mm
Column temperature: 25° C.
Eluents: A: TFA (0.05%) in water
   B: TFA (0.05%) in ACN
Flow rate: 0.3 mL/min
Gradient: T/% B 0/60, 2/90, 8/90; Run Time: 8 min
Detection ELSD, ESI (+/−), 100-1200 m/z

### LCMS Method G4:

Column: Waters Acquity UPLC® BEH C18, 1.7 μm, 2.1×100 mm
Column temperature: 25° C.
Eluents: A: TFA (0.05%) in Water
   B: TFA (0.05%) in ACN
Flow rate: 0.3 mL/min
Gradient: T/% B 0/60, 2/90, 6/90; Run Time: 6 min

### LCMS Method G5:

Column: Waters Acquity UPLC® BEH C18, 1.7 μm, 2.1×100 mm
Column temperature: 25° C.
Eluents: A: TFA (0.05%) in water
   B: TFA (0.05%) in ACN
Flow rate: 0.3 mL/min
Gradient: T/% B 0/30, 4/90, 8/90; Run Time: 8 min
Detection ELSD

### LCMS Method G6:

Column: Waters Acquity UPLC® BEH C18, 1.7 μm, 2.1×50 mm
Column temperature: 35° C.
Eluents: A: TFA (0.05%) in water
   B: TFA (0.05%) in ACN
Flow rate: 0.4 mL/min
Gradient: T/% B 0/10, 0.5/10, 1/35, 1.5/45, 2.3/90, 3.2/90, 3.6-4.0/10;
Run Time: 4 min
Detection ELSD

### LCMS Method G7:

Column: Waters Xbridge BEH C18, 2.5 μm, 2.1×50 mm
Column temperature: 40° C.
Eluents: A: 10 mM Ammonium Formate in Water
   B: ACN
Flow rate: 0.7 mL/min
Gradient: T/% B 0/20, 5/100, 8/100, 8.5/20, 10/20; Run Time: 10 min
Detection ELSD, DAD, ESI (+/−)

### Chiral Method G8:

Column: Chiralpak AD-H, 5 μm, 4.6×250 mm
Column temperature: 30° C.
SFC Conditions: Isocratic, 30% over 10 min, 100 bar, 30% MeOH+0.5% DEA; Run Time: 10 min
Flow rate: 3 mL/min
Detection UV/VIS 214 nm

### Chiral Method G9:

Column: Chiralpak AD-H, 5 μm, 4.6×250 mm
Column temperature: 30° C.
SFC Conditions: Isocratic, 30% over 10 min, 100 bar, 30% MeOH+0.2% DEA; Run Time: 10 min
Flow rate: 3 mL/min
Detection UV/VIS 214 nm

### LCMS-Method CB-A:

Column: Chiralpak IC, 5 μm, 4.6×250 mm
Column temperature: RT
Eluents: Isocratic, TBME:DCM:MeOH:water:DEA (52:18:28:2:0.1)
Run Time: 60 min
Flow rate: 1 mL/min
Detection UV 240 nm

### Method Chiral Prep SFC:

Instrument: Thar 80
Column: Chiralpak IG, 250×21 mm I.D.
Mobile phase: A for CO₂ and B for Isopropanol
Gradient: Isocratic B 21%
Flow rate: 80 mL/min
Back pressure: 1 50 bar
Column temperature: RT
Wavelength: 226 nm

### Achiral Preparative HPLC Methods:

Waters AutoPurification System with PDA (photodiode array detector) and single quadrupole mass detector with ESI ionization.

### Instrument: CA-Method 5:

| Pump | Waters 2545 | | |
|---|---|---|---|
| Fraction Collector | Waters 2767 | | |
| Modifier Pump | Waters 515 | | |
| Detector | Waters 2998 | | |
| Mass Spec | Waters SQD-2 | | |
| Columns | Waters XBridge BEH C18 | | |
| | OBD 5 μm 30 × 50 mm | | |
| Eluent A | water | | |
| Eluent B | ACN | | |
| Acidic Modifier | 375 mM Formic Acid | | |
| Flow | 75 mL/min | | |
| Stop Time | 6.5 min | | |
| Gradient | Time | % A (Eluent A) | % B (Eluent B) |
| | 0.00 | 65 | 35 |
| | 1.00 | 65 | 35 |
| | 4.20 | 40 | 60 |
| | 4.70 | 5 | 95 |
| | 6.00 | 5 | 95 |
| | 6.40 | 65 | 35 |
| | 6.50 | 65 | 35 |

| | |
|---|---|
| Column Temperature | RT |
| UV | 210-400 nm |

### Abbreviations

ACN acetonitrile
aq. aqueous
Ar argon
Boc tertiary butyl carboxy
Bn benzyl
BnOH benzyl alcohol
br broad
cataCXium® A Pd G3 Mesylate[(di(1-adamantyl)-n-butylphosphine)-2-(2′-amino-1,1′-biphenyl)]palladium(II), [(Di(1-adamantyl)-butylphosphine)-2-(2′-amino-1,1′-biphenyl)]palladium(II) methanesulfonate
Cs₂CO₃ cesium carbonate
d doublet
DCC N,N-dicyclohexylcarbodiimide
DIC N,N-Diisopropylcarbodiimide
DCM dichloromethane
dd doublet of doublets
DEA diethanolamine
DIPE diisopropyl ether
DIPEA Diisopropylethylamine
DMA N,N-dimethylacetamide
DMAP N,N-dimethylaminopyridine
DMF N,N-dimethylformamide
DMSO dimethylsulfoxide
DSC Di(N-succinimidyl) carbonate
EDCl 1-ethyl-3-(3-dimethylaminopropyl)carbodiimide
ELSD evaporative light scattering detector
ee enantiomeric excess
ent enantiopure
Et₂O diethyl ether
EtOAc ethyl acetate
EtOH ethanol
FCC Flash Column Chromatography
h hour(s)
H₂ hydrogen
HCl hydrochloric acid
HATU 1-[Bis(dimethylamino)methylene]-1H-1,2,3-triazolo[4,5-b]pyridinium 3-oxid hexafluorophosphate
HPLC high performance liquid chromatography
HRMS high-resolution mass spectrometry
IPA isopropanol
i.v. intravenous infusion
LCMS liquid chromatography and mass spectrometry
m multiplet
m/z mass to charge ratio
MCC microcrystalline cellulose
MeOH methanol
2-MeTHF 2-methyltetrahydrofuran
MgSO₄ magnesium sulfate
min minutes
MS mass spectrometry
MsCl methanesulfonyl chloride
MTBE methyl t-butyl ether
N₂ nitrogen
NaBH(OAc)₃ sodium triacetoxyborohydride
NaBH₃CN sodium cyanoborohydride
NaCl sodium chloride
NaH sodium hydride
NaHCO₃ sodium bicarbonate
NaOMe sodium methoxide
Na₂SO₄ sodium sulfate
NMR nuclear magnetic resonance
Pd/C palladium on carbon
PdCl₂(dppf) [1,1′-Bis(diphenylphosphino)ferrocene]palladium(II) dichloride
PdCl₂(PPh₃)₂ Bis(triphenylphosphine)palladium(II) dichloride
PE petroleum ether
Pfp Pentafluorophenyl
PPh₃ triphenylphosphine
PPh₃O triphenylphosphine oxide
ppm parts per million
p/s photons/second
PTFE polytetrafluoroethylene
qw once a week
q2W every two weeks
q3W every three weeks
quin quintet
rac racemic
RM reaction mixture
RP Reverse-Phase
Rt retention time
RT room temperature
s singlet
s.c. subcutaneous
sat. saturated
SFC Supercritical Fluid Chromatography
SiO₂ silica
t triplet
TAC Total Absorbance Chromatogram
TBME tert-butyl methyl ether
TEA triethylamine
TFA trifluoroacetic acid
THF tetrahydrofuran
TLC thin layer chromatography
UPLC ultra performance liquid chromatography
   Compounds of the present disclosure can be prepared as described in the following Examples.

### Intermediates V

### Example 1a: 3-((2-(Benzyloxy)-2-oxoethyl)(tert-butoxycarbonyl)amino)propanoic acid (Intermediate V1)

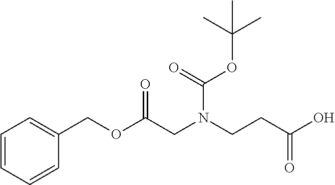 [SMILES: CC(C)(C)OC(=O)N(CCC(=O)O)CC(=O)OCc1ccccc1]

Two parallel reactions with benzyl glycinate, HCl-salt (40 g+40 g, 397 mmol). To a suspension of benzyl glycinate, HCl-salt (40 g, 198 mmol) in ACN (700 mL), at RT under Ar, was added DIPEA (35 mL, 200 mmol). The reaction was warmed to 45° C. whereupon all aggregates disappeared to leave a colorless cloudy reaction. Acrylic acid (16.5 mL, 241 mmol) was added dropwise over 25 min. The reaction was stirred at 45° C. for an additional 45 min before being allowed to cool down to RT. To the mixture was added di-tert-butyl dicarbonate (44 g, 202 mmol) and ACN (30 mL). The reaction was stirred at RT for an additional 23 h, concentrated under reduced pressure, taken up in EtOAc (400 mL) and washed with a 0.25 M aq. solution of HCl (400 mL). The aq. layer was extracted once with EtOAc (200 mL). The combined organic layers were washed with a 1:1 mixture of water and brine (300 mL). The organic layer was dried over MgSO₄, filtered, concentrated under reduced pressure, dried and purified by chromatography on RediSep® Gold silica column (330 g) eluting with EtOAc (from 15% to 100%) in cyclohexane to afford the title compound Intermediate V1 as a colorless/slightly yellow oil. The material from the two parallel reactions were combined providing a total of 54.4 g. LCMS Method W1: Rt=4.68 min; MS m/z [M−H]⁻=336.2.

### Example 2a: Perfluorophenyl 3-(2,4-dioxotetrahydropyrimidin-1(2H)-yl)-4-methoxybenzoate (Intermediate V2)

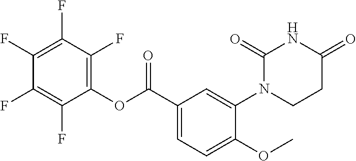 [SMILES: COc1ccc(C(=O)Oc2c(F)c(F)c(F)c(F)c2F)cc1N1CCC(=O)NC1=O]

### Step 1: 3-(2,4-Dioxotetrahydropyrimidin-1(2H)-yl)-4-methoxybenzoic acid (Intermediate V2-1)

3-Amino-4-methoxybenzoic acid (5.0 g, 29.3 mmol) was suspended in acrylic acid (8.05 mL, 117 mmol) and the resulting suspension was stirred at 100° C. for 3 h. The RM was allowed to cool to RT, AcOH (33 mL) was added and the stirred suspension was heated at 100° C. for 10 min. Urea (11.0 g, 183 mmol) was added and the RM was stirred at 120° C. overnight. The solution was poured into an ice cold mixture of water and concentrated aq. HCl (37%). After stirring, the resulting suspension was stored overnight in the fridge at 5° C., then filtered and the solids were washed with water and dried to afford a solid. The solid was triturated in an aq. solution of HCl (0.05 M) and filtered off. The solids were washed with TBME and dried at 40° C. under reduced pressure to afford the title compound Intermediate V2-1 (6.29 g) as a solid. LCMS Method T1 Rt=0.48 min; [M+H]⁺=265.2.

### Step 2: Perfluorophenyl 3-(2,4-dioxotetrahydropyrimidin-1(2H)-yl)-4-methoxybenzoate (Intermediate V2)

3-(2,4-Dioxotetrahydropyrimidin-1(2H)-yl)-4-methoxybenzoic acid (Intermediate V2-1, 18 g, 68.1 mmol) and DIPEA (23.8 mL, 136 mmol) were suspended in 170 mL anhydrous DMF. The suspension was cooled to 0° C. Perfluorophenyl 2,2,2-trifluoroacetate (17.92 mL, 102 mmol) was added in a continuous stream. The resulting solution was stirred for 3 h and then the reaction volume was reduced by half under reduced pressure. Upon slight cooling, the viscous liquid set to a semicrystalline mass. To this residue was added 600 mL Et₂O which caused further precipitation. This suspension was stirred for additional 30 min then filtered off. The resulting filtered solid was washed with Et₂O (2×200 mL) and dried under reduced pressure providing the title compound Intermediate V2 (21.27 g) as a fluffy microcrystalline solid. Method T7: Rt=1.03 min, [M+H]⁺=431.4.

### Example 3a: 1-(5-(9-(4-(4-Chloro-7H-pyrrolo[2,3-d]pyrimidin-6-yl)phenethyl)-3,9-diazaspiro[5.5]undecane-3-carbonyl)-2-methoxyphenyl)dihydropyrimidine-2,4(1H,3H)-dione (Intermediate V3)

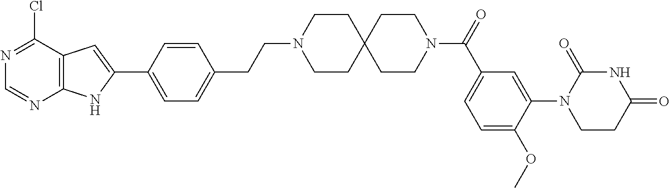 [SMILES: COc1ccc(C(=O)N2CCC3(CCN(CCc4ccc(-c5cc6c(Cl)ncnc6[nH]5)cc4)CC3)CC2)cc1N1CCC(=O)NC1=O]

### Step 1: 2-(4-(4,4,5,5-Tetramethyl-1,3,2-dioxaborolan-2-yl)phenyl)ethan-1-ol (Intermediate V3-1)

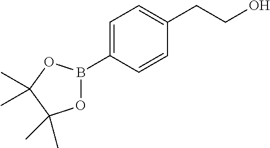 [SMILES: CC1(C)OB(c2ccc(CCO)cc2)OC1(C)C]

A mixture of 2-(4-bromophenyl)ethan-1-ol (60 g, 300 mmol), bis(pinacolato)diboron (84 g, 330 mmol), KOAc (90 g, 900 mmol) and PdCl₂(dppf) (6.6 g, 9 mmol) in 1,4-dioxane (600 mL) was stirred under N₂ at 85° C. for 16 h. The RM was cooled to RT, filtered and then concentrated under reduced pressure. The resulting residue was purified by chromatography on silica gel eluting with EtOAc in PE (0 to 30%) to afford the title compound Intermediate V3-1 (100 g) as an oil. LCMS Method W2: Rt=1.87 min, [M+NH₄]+=266.

### Step 2: 4-(4,4,5,5-Tetramethyl-1,3,2-dioxaborolan-2-yl)phenethyl methanesulfonate (Intermediate V3-2)

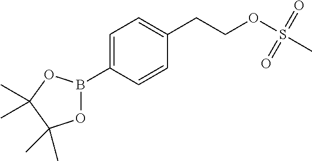

A mixture of 2-(4-(4,4,5,5-tetramethyl-1,3,2-dioxaborolan-2-yl)phenyl)ethan-1-ol (Intermediate V3-1, 100 g, 300 mmol) and TEA (240 g, 2.40 mol) in DCM (1.3 L) was stirred at 0° C. for 20 min. A solution of MsCl (136 g, 1.20 mol) in DCM (200 mL) was added dropwise and the RM was stirred at RT for 16 h. Water was added and after extraction, the phases were separated, the organic phase was dried over Na₂SO₄, concentrated under reduced pressure and the residue purified by chromatography on silica gel eluting with EtOAc in DCM (0 to 50%) to afford the title compound Intermediate V3-2 (87 g) as an oil. LCMS Method W3: Rt=1.92 min, [M+H]⁺=327.

### Step 3: tert-Butyl 9-(4-(4,4,5,5-tetramethyl-1,3,2-dioxaborolan-2-yl)phenethyl)-3,9-diazaspiro[5.5]undecane-3-carboxylate (Intermediate V3-3)

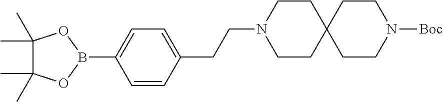 [SMILES: CC(C)(C)OC(=O)N1CCC2(CCN(CCc3ccc(B4OC(C)(C)C(C)(C)O4)cc3)CC2)CC1]

A mixture of tert-butyl 3,9-diazaspiro[5.5]undecane-3-carboxylate (34 g, 133 mmol), 4-(4,4,5,5-tetramethyl-1,3,2-dioxaborolan-2-yl)phenethyl methanesulfonate (86 g, 172 mmol), K₂CO₃ (47 g, 345 mmol) and potassium iodide (2.3 g, 13.8 mmol) in ACN (1 L) was stirred at 60° C. for 16 h. The RM was filtered and the filtrate was concentrated under reduced pressure. The residue was purified by chromatography on silica gel eluting with MeOH in DCM (0 to 10%) to give the title compound Intermediate V3-3 (49 g) as a solid. LCMS Method W4: Rt=1.47 min, [M+H]⁺=485.

### Step 4: tert-Butyl 9-(4-(4-chloro-7-(phenylsulfonyl)-7H-pyrrolo[2,3-d]pyrimidin-6-yl)phenethyl)-3,9-diazaspiro[5.5]undecane-3-carboxylate (Intermediate V3-4)

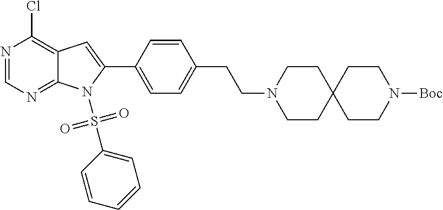 [SMILES: CC(C)(C)OC(=O)N1CCC2(CCN(CCc3ccc(-c4cc5c(Cl)ncnc5n4S(=O)(=O)c4ccccc4)cc3)CC2)CC1]

A mixture of tert-butyl 9-(4-(4,4,5,5-tetramethyl-1,3,2-dioxaborolan-2-yl)phenethyl)-3,9-diazaspiro[5.5]undecane-3-carboxylate (Intermediate V3-3, 38 g, 79 mmol), 4-chloro-6-iodo-7-(phenylsulfonyl)-7H-pyrrolo[2,3-d]pyrimidine (37 g, 88 mmol), K₂CO₃ (22 g, 160 mmol) and PdCl₂(dppf) (5.8 g, 8 mmol) in a mixture of 1,4-dioxane and water (5:1) (480 mL) was stirred under an atmosphere of N₂ at 80° C. for 16 h. The RM was poured into EtOAc, the organic phase washed with water, dried over Na₂SO₄, filtered, concentrated under reduced pressure. The residue was purified by chromatography on silica gel eluting with MeOH in DCM (0 to 10%) to give the title compound Intermediate V3-4 (34 g) as a solid. LCMS Method W4: Rt=1.99 min, [M+H]⁺=650.

### Step 5: tert-Butyl 9-(4-(4-chloro-7H-pyrrolo[2,3-d]pyrimidin-6-yl)phenethyl)-3,9-diazaspiro[5.5]undecane-3-carboxylate (Intermediate V3-5)

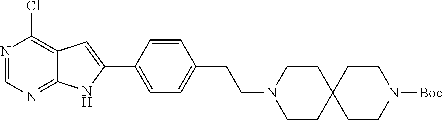 [SMILES: CC(C)(C)OC(=O)N1CCC2(CCN(CCc3ccc(-c4cc5c(Cl)ncnc5[nH]4)cc3)CC2)CC1]

In a 5-L reaction vessel at RT and under N₂ atmosphere, tert-butyl 9-(4-(4-chloro-7-(phenylsulfonyl)-7H-pyrrolo[2,3-d]pyrimidin-6-yl)phenethyl)-3,9-diazaspiro[5.5]undecane-3-carboxylate (Intermediate V3-4, 101.3 g, 156 mmol) was dissolved in 2 L anhydrous THF. A solution of TBAF in THF (1M, 156 mL, 156 mmol) was added. The RM was mechanically stirred at RT under N₂ atmosphere for 1.5 h. The RM was concentrated under reduced pressure. The resulting oil was poured into 2 L EtOAc and treated with 1.5 L of sat. aq. ammonium chloride while rapidly stirring. The resulting precipitate was filtered off. The filtrate was washed with EtOAc and dried briefly on the filtration frit to yield the title compound Intermediate V3-5 (71.36 g). LCMS Method T8: Rt=0.90 min, [M+H]⁺=510.6.

### Step 6: 3-(4-(4-Chloro-7H-pyrrolo[2,3-d]pyrimidin-6-yl)phenethyl)-3,9-diazaspiro[5.5]undecane (Intermediate V3-6)

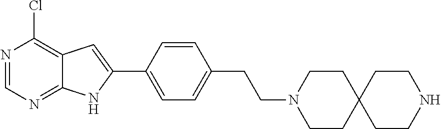 [SMILES: Clc1ncnc2[nH]c(-c3ccc(CCN4CCC5(CCNCC5)CC4)cc3)cc12]

To a solution of tert-butyl 9-(4-(4-chloro-7H-pyrrolo[2,3-d]pyrimidin-6-yl)phenethyl)-3,9-diazaspiro[5.5]undecane-3-carboxylate (Intermediate V3-5, 70.4 g, 138 mmol) in anhydrous DCM (1.1 L) was added slowly TFA (162 mL, 2.1 mol). After stirring for 1 h the volatiles were removed carefully under reduced pressure yielding a brown oil. The residual TFA was azeotroped off by adding toluene (1 L) and concentrating under reduced pressure to provide the title compound Intermediate V3-6 which was directly used in the next step without purification. LCMS Method T8 Method: Rt=0.62 min, [M+H]⁺=410.5.

### Step 7: 1-(5-(9-(4-(4-Chloro-7H-pyrrolo[2,3-d]pyrimidin-6-yl)phenethyl)-3,9-diazaspiro[5.5]undecane-3-carbonyl)-2-methoxyphenyl)dihydropyrimidine-2,4(1H,3H)-dione (Intermediate V3)

3-(4-(4-Chloro-7H-pyrrolo[2,3-d]pyrimidin-6-yl)phenethyl)-3,9-diazaspiro[5.5]undecane (56.6 g) was dissolved into anhydrous DMF (276 mL) and treated with DIPEA (241 mL, 1.38 mol). Under ice bath cooling perfluorophenyl 3-(2,4-dioxotetrahydropyrimidin-1(2H)-yl)-4-methoxybenzoate (59.4 g, 138 mmol) was added to the RM and the mixture was allowed to stir at RT for 16 h. The volatiles were removed under reduced pressure and the resulting residue was treated with EtOAc (1.5 L) under rapid stirring. Stirring was continued for 1 h and the solids were filtered, washed with EtOAc (500 mL) and dried under high vacuum at 40° C. for 3 days providing the title compound Intermediate V3 (8.1 g). LCMS Method T8 Method: Rt=0.75 min, [M+H]⁺=656.7.

### Example 4a: 1-(5-(9-(4-(4-(3-amino-5-fluoro-2-methylphenyl)-7H-pyrrolo[2,3-d]pyrimidin-6-yl)phenethyl)-3,9-diazaspiro[5.5]undecane-3-carbonyl)-2-methoxyphenyl)dihydropyrimidine-2,4(1H,3H)-dione (Intermediate V4)

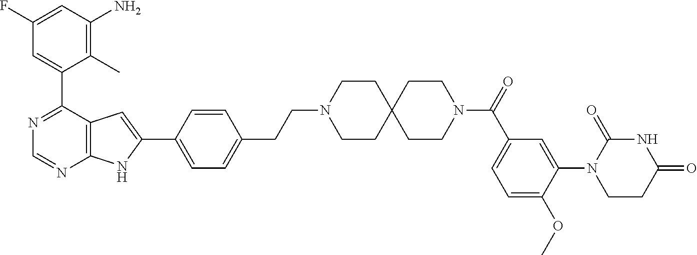 [SMILES: COc1ccc(C(=O)N2CCC3(CCN(CCc4ccc(-c5cc6c(-c7cc(F)cc(N)c7C)ncnc6[nH]5)cc4)CC3)CC2)cc1N1CCC(=O)NC1=O]

To a solution of 1-(5-(9-(4-(4-chloro-7H-pyrrolo[2,3-d]pyrimidin-6-yl)phenethyl)-3,9-diazaspiro[5.5]undecane-3-carbonyl)-2-methoxyphenyl)dihydropyrimidine-2,4(1H, 3H)-dione (Intermediate V3, 1.323 g, 2.016 mmol) and 5-fluoro-2-methyl-3-(4,4,5,5-tetramethyl-1,3,2-dioxaborolan-2-yl)aniline (1.266 g, 5.04 mmol) in dioxane (27.8 mL) and water (12.52 mL) was added an aq. NaHCO₃ solution (2M, 2.52 mL, 5.04 mmol). The RM was degassed with N₂ for several min. PdCl₂(dppf)-CH₂Cl₂ (0.165 g, 0.202 mmol) was added to the mixture and degassing with N₂ was continued for 2 min. The RM was heated at 80° C. in a pre-heated bath for 15 min. The RM was cooled down to RT and filtered through a plug of Celite® and washed with dioxane. The filtrate was concentrated under reduced pressure. The resulting residue was dissolved in 10 mL MeOH (10 mL) and purified by reverse phase ISCO eluting with ACN/water 10-35% (0.1% TFA) over a 150 g C18 RediSep® Rf column. The product fractions were lyophilized to provide Intermediate V4, (955 mg) as a cream fluffy solid. LCMS Method W10: Rt=0.65 min., MS m/z [M+H]⁺=745.5.

### Example 5a: N-(3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide (Compound V)

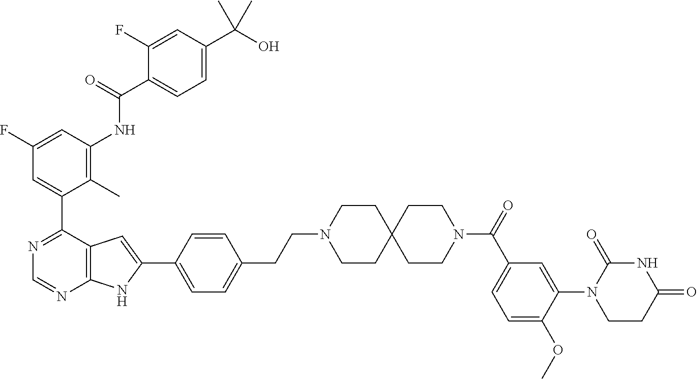 [SMILES: COc1ccc(C(=O)N2CCC3(CCN(CCc4ccc(-c5cc6c(-c7cc(F)cc(NC(=O)c8ccc(C(C)(C)O)cc8F)c7C)ncnc6[nH]5)cc4)CC3)CC2)cc1N1CCC(=O)NC1=O]

The preparation of Compound V is described in WO2019/186343.

### Example 5b: (3R,4S)—N-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-3-hydroxy-4-isobutylpyrrolidine-1-carboxamide (Compound Va)

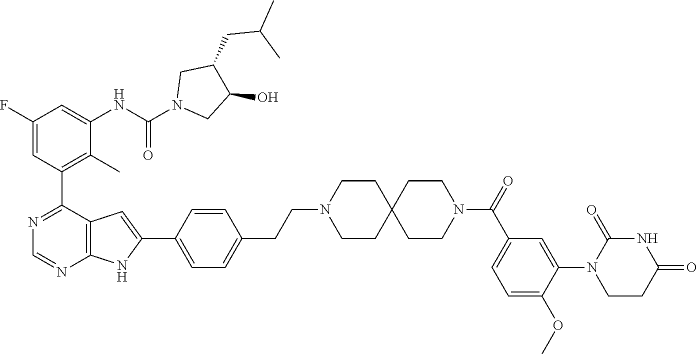 [SMILES: COc1ccc(C(=O)N2CCC3(CCN(CCc4ccc(-c5cc6c(-c7cc(F)cc(NC(=O)N8C[C@H](CC(C)C)[C@@H](O)C8)c7C)ncnc6[nH]5)cc4)CC3)CC2)cc1N1CCC(=O)NC1=O]

The preparation of Compound Va is described in WO2019/186358.

### Intermediates S

### Example 1b: 4-(2-((3-((tert-Butoxycarbonyl)amino)-2,2-dimethylpropanoyl)oxy)propan-2-yl)-2-fluorobenzoic acid (Intermediate S1)

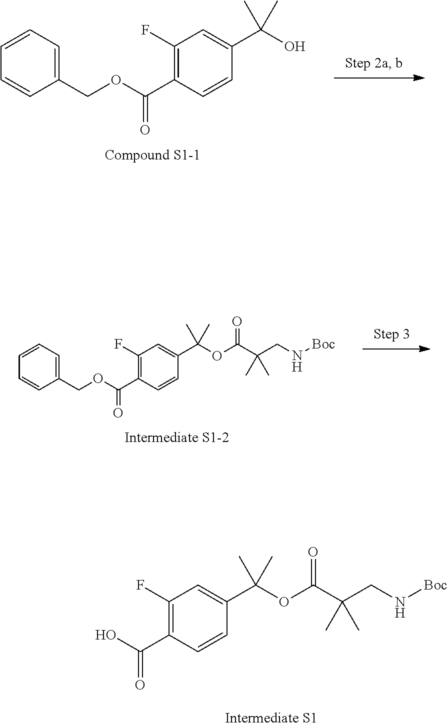 [SMILES: CC(C)(O)c1ccc(C(=O)OCc2ccccc2)c(F)c1 ; CC(C)(C)OC(=O)NCC(C)(C)C(=O)OC(C)(C)c1ccc(C(=O)OCc2ccccc2)c(F)c1 ; CC(C)(C)OC(=O)NCC(C)(C)C(=O)OC(C)(C)c1ccc(C(=O)O)c(F)c1]

### Step 1: Benzyl 2-fluoro-4-(2-hydroxypropan-2-yl)benzoate (Intermediate S1-1)

To a solution of 2-fluoro-4-(2-hydroxypropan-2-yl)benzoic acid (21 g, 106 mmol) in DMF (400 mL) was added cesium carbonate (38.0 g, 117 mmol) at 0° C. The RM was stirred for 5 min and benzyl bromide (13.23 mL, 111 mmol) was added dropwise. The reaction vessel was removed from the ice bath after 5 min and stirring was continued at RT for 1 h. The RM was poured into water (500 mL) and was extracted with EtOAc (3×500 mL). The organic layers were combined, dried over Na₂SO₄, filtered, and concentrated under reduced pressure. The crude residue was purified by chromatography over silica gel (eluting with EtOAc/Heptane, 0 to 40%) providing Intermediate S1-1 (28.7 g) as a colorless liquid. ¹H NMR (400 MHz, DMSO-d₆) δ [ppm]7.92-7.81 (m, 1H), 7.50-7.31 (m, 7H), 5.40-5.29 (m, 3H), 1.42 (s, 6H).

### Step 2a: Benzyl 4-(2-((3-((tert-butoxycarbonyl)amino)-2-methylpropanoyl)oxy)propan-2-yl)-2-fluorobenzoate (Intermediate S1-2a)

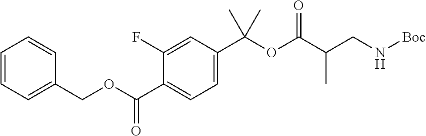 [SMILES: CC(CNC(=O)OC(C)(C)C)C(=O)OC(C)(C)c1ccc(C(=O)OCc2ccccc2)c(F)c1]

To a mixture of benzyl 2-fluoro-4-(2-hydroxypropan-2-yl)benzoate (Intermediate S1-1, 1.39 g, 4.82 mmol) and 3-((tert-butoxycarbonyl)amino)-2-methylpropanoic acid (0.980 g, 4.82 mmol) in DCM (12 mL) was added HATU (2.75 g, 7.23 mmol) and DMAP (2.061 g, 16.87 mmol). The mixture was stirred in a sealed vial under N₂ atmosphere for ˜4 days. The RM was diluted with Et₂O (˜24 mL) and vigorously stirred for 5-10 min. The solids were filtered off and rinsed with Et₂O. The filtrate was concentrated under reduced pressure. The residue was dissolved in EtOAc and washed with sat. aq. NaHCO₃ solution, brine, dried over Na₂SO₄ and concentrated under reduced pressure. The residue was purified by column chromatography over silica gel (120 g, EtOAc/heptane=0 to 40%) providing the title compound Intermediate S1-2a (1.9 g) as a colorless oil. LCMS Method T7: Rt=1.29 min; MS m/z [M+H]⁺=474.2.

### Step 2b: Benzyl 4-(2-((3-((tert-butoxycarbonyl)amino)-2,2-dimethylpropanoyl)oxy)propan-2-yl)-2-fluorobenzoate (Intermediate S1-2)

To benzyl 4-(2-((3-((tert-butoxycarbonyl)amino)-2-methylpropanoyl)oxy)propan-2-yl)-2-fluorobenzoate (1.9 g, 4.01 mmol) in anhydrous THF (40 mL) and under N₂ atmosphere was slowly added lithium diisopropylamide (4.01 mL, 8.02 mmol) at −78° C. and stirring was continued at this temperature for ˜20 min. To the mixture was added dropwise iodomethane (0.502 mL, 8.02 mmol) and stirring was continued for ˜30 min. An equal amount of water (˜0.5 mL) was added and the vigorously stirring mixture was allowed to warm to RT. After dilution with EtOAc the organic layer was washed with brine, dried over Na₂SO₄, filtered and concentrated under reduced pressure. The resulting residue was purified by column chromatography (SiO₂, 120 g, eluting with EtOAc/heptane=0/100 to 30/70) providing the title compound (Intermediate S1-2, 350 mg). LCMS Method T7: Rt=1.34 min; MS m/z [M+H]⁺=488.2.

### Step 3: 4-(2-((3-((tert-Butoxycarbonyl)amino)-2,2-dimethylpropanoyl)oxy)propan-2-yl)-2-fluorobenzoic acid (Intermediate S1)

To a solution of benzyl 4-(2-((3-((tert-butoxycarbonyl)amino)-2,2-dimethylpropanoyl)oxy)propan-2-yl)-2-fluorobenzoate (Intermediate S1-2, 350 mg, 0.718 mmol) in EtOAc (6 mL) was added Pd/C (153 mg, 0.144 mmol, 10 wt. %) under N₂ atmosphere. The atmosphere was replaced with H₂ from a balloon reservoir and the mixture was stirred for ˜2.5 h at 25° C. Celite® were added to the RM and the mixture was filtered over Celite®. The filtrate was concentrated under reduced pressure and the residue was dissolved in DCM. The solution was concentrated under reduced pressure to provide the crude title compound Intermediate S1, which was directly used in next reaction without further purification. LCMS Method T7: Rt=1.04 min; MS m/z [M+H]⁺=398.3.

### Example 2b: 4-(2-(((tert-Butoxycarbonyl)alanyl)oxy)propan-2-yl)-2-fluorobenzoic acid (Intermediate S2)

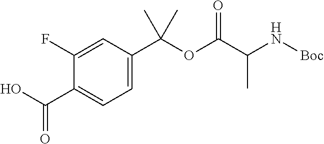 [SMILES: CC(NC(=O)OC(C)(C)C)C(=O)OC(C)(C)c1ccc(C(=O)O)c(F)c1]

Intermediate S2 was prepared according to the procedure described for Intermediate S1, Step 1 and Step 3, above in Example 1b, using rac Boc-alanine. Step 1: benzyl 4-(2-(((tert-butoxycarbonyl)alanyl)oxy)propan-2-yl)-2-fluorobenzoate-LCMS Method T7: Rt=1.37 min; MS m/z [M+H−tBu]⁺=404.2. Step 2: 4-(2-(((tert-butoxycarbonyl)alanyl)oxy)propan-2-yl)-2-fluorobenzoic acid (Intermediate S2)-LCMS Method T7: Rt=1.07 min; MS m/z [M−H]⁻=368.3.

### Example 3b: 4-(2-(((tert-Butoxycarbonyl)-D-alanyl)oxy)propan-2-yl)-2-fluorobenzoic acid (Intermediate S3) and 4-(2-(((tert-Butoxycarbonyl)-L-alanyl)oxy)propan-2-yl)-2-fluorobenzoic acid (Intermediate S4)

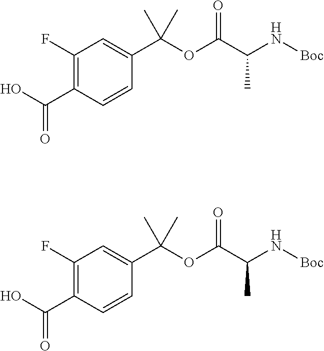 [SMILES: C[C@@H](NC(=O)OC(C)(C)C)C(=O)OC(C)(C)c1ccc(C(=O)O)c(F)c1 ; C[C@H](NC(=O)OC(C)(C)C)C(=O)OC(C)(C)c1ccc(C(=O)O)c(F)c1]

SFC Chiral purification (Method Chiral Prep SFC) of rac 4-(2-(((tert-butoxycarbonyl)-alanyl)oxy)propan-2-yl)-2-fluorobenzoic acid (5.4 g) provided Intermediate S3 (2.5 g; Peak 1; Rt=2.6 min). LCMS Method T7: Rt=1.08 min; MS m/z [M−H]⁻=363.8 and Intermediate S4 (2.5 g; Peak 2; Rt=4.3 min). LCMS Method T7: Rt=1.07 min; MS m/z [M−H]⁻=363.8.

The elution order of the enantiomers was identified by performing the reaction sequence as described for Intermediate S2, above, using D-Boc-alanine and L-Boc-alanine, respectively, which provided the partially isomerized corresponding acid derivatives.

### Intermediates B

### Example 1c: 2-Fluoro-N-(5-fluoro-2-methyl-3-(4,4,5,5-tetramethyl-1,3,2-dioxaborolan-2-yl)phenyl)-4-(2-hydroxypropan-2-yl)benzamide (Intermediate B1)

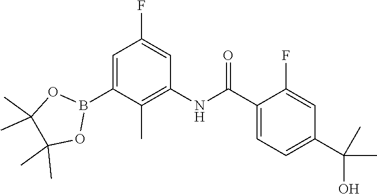 [SMILES: Cc1c(NC(=O)c2ccc(C(C)(C)O)cc2F)cc(F)cc1B1OC(C)(C)C(C)(C)O1]

A mixture of 2-fluoro-4-(2-hydroxypropan-2-yl)benzoic acid (6.35 g, 32.0 mmol), HATU (17.06 g, 44.9 mmol) and DIPEA (16.79 mL, 96 mmol) in DMF (100 mL) was stirred at RT for 30 min. Then, 5-fluoro-2-methyl-3-(4,4,5,5-tetramethyl-1,3,2-dioxaborolan-2-yl)aniline (can be prepared according to the procedure described in published PCT application WO2013/008095 A1, page 37, intermediate 5) (8.47 g, 32.0 mmol) was added and the RM was stirred at 50° C. overnight. The RM was diluted with EtOAc and the organic phase was washed with a sat. aq. solution of NaHCO₃ and brine. The combined aq. phases were extracted again with EtOAc and the combined organic phases were dried over Na₂SO₄, filtered, concentrated under reduced pressure. The residue was purified by chromatography on silica gel eluting with MeOH in DCM (from 0 to 10%) and the resulting solid was triturated with Et₂O and filtered. The solids were washed with diisopropylether and dried to afford the title compound Intermediate B1 as a solid (9.28 g). LCMS Method T1: Rt=1.28 min; [M+H]⁺=432.3. [An alternative preparation of Intermediate B1 is described in WO2013/008095 A1, page 81, intermediate 35.]

### Example 2c: 2-(3-Fluoro-4-((5-fluoro-2-methyl-3-(4,4,5,5-tetramethyl-1,3,2-dioxaborolan-2-yl)phenyl)carbamoyl)phenyl)propan-2-yl 3-((tert-butoxycarbonyl)amino)propanoate (Intermediate B2)

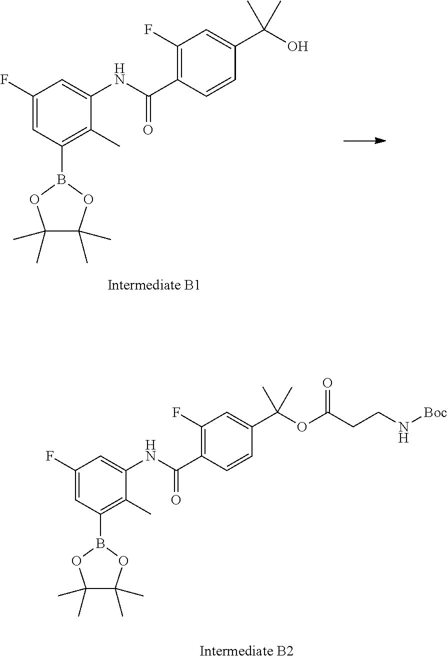 [SMILES: Cc1c(NC(=O)c2ccc(C(C)(C)O)cc2F)cc(F)cc1B1OC(C)(C)C(C)(C)O1 ; Cc1c(NC(=O)c2ccc(C(C)(C)OC(=O)CCNC(=O)OC(C)(C)C)cc2F)cc(F)cc1B1OC(C)(C)C(C)(C)O1]

To a suspension of 2-fluoro-N-(5-fluoro-2-methyl-3-(4,4,5,5-tetramethyl-1,3,2-dioxaborolan-2-yl)phenyl)-4-(2-hydroxypropan-2-yl)benzamide (Intermediate B1, 10.7 g, 24.81 mmol) in DCM (75 mL), at RT under Ar atmosphere, were added 3-((tert-butoxycarbonyl)amino)propanoic acid (6.10 g, 32.3 mmol), DMAP (3.94 g, 32.3 mmol) and DCC (7.68 g, 37.2 mmol). The reaction was stirred at RT for 2 days and then filtered. The white solid was washed with DCM. The filtrate was concentrated under reduced pressure, absorbed on silica and purified by chromatography on silica gel (220 g) eluting with EtOAc (from 10% to 100%) in cyclohexane to afford the title compound Intermediate B2 (7.57 g) as a white foam. LCMS Method T2: Rt=1.49 min; MS m/z [M+H]⁺=603.4.

Alternatively, acylation of the hydroxyl group can be carried out using DCC instead of HATU, e.g., Intermediate B9 in Table 5, herein below.

### Example 3c: 2-(3-Fluoro-4-((5-fluoro-2-methyl-3-(4,4,5,5-tetramethyl-1,3,2-dioxaborolan-2-yl)phenyl)carbamoyl)phenyl)propan-2-yl (tert-butoxycarbonyl)glycinate (Intermediate B3)

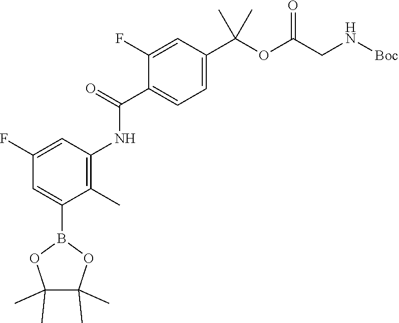 [SMILES: Cc1c(NC(=O)c2ccc(C(C)(C)OC(=O)CNC(=O)OC(C)(C)C)cc2F)cc(F)cc1B1OC(C)(C)C(C)(C)O1]

To a solution of 2-fluoro-N-(5-fluoro-2-methyl-3-(4,4,5,5-tetramethyl-1,3,2-dioxaborolan-2-yl)phenyl)-4-(2-hydroxypropan-2-yl)benzamide (Intermediate B1, 2000 mg, 4.64 mmol) in anhydrous DCM (12 mL), at RT and under Ar atmosphere were added DMAP (736 mg, 6.03 mmol), DCC (1435 mg, 6.96 mmol) and (tert-butoxycarbonyl)glycine (1056 mg, 6.03 mmol). The suspension was stirred at RT for 4.5 h. The mixture was absorbed onto silica and purified by chromatography on silica gel (80 g) eluting with EtOAc (from 0% to 100%) in cyclohexane to afford the title compound Intermediate B3 (1.21 g) as a white foam containing 18% {by LCMS UV detector TAC 210-450 nm} of bis-acylated material. Major component: ¹H NMR (400 MHz, DMSO-d₆) δ [ppm] 9.88 (s, 1H), 7.76-7.63 (m, 1H), 7.58-7.45 (m, 1H), 7.42-7.29 (m, 2H), 7.27-7.18 (m, 2H), 3.69 (d, J=6.2 Hz, 2H), 2.39 (s, 3H), 1.72 (s, 6H), 1.38 (s, 9H), 1.32 (s, 12H). LCMS Method T2: Rt=1.43 min; [M−H]⁻=587.4; Minor component: Rt=1.53 min [M+H]⁺=746.4.

The following compounds in Table 5 were prepared from Intermediate B1 and corresponding acids according to the procedures described for Intermediate B2 and Intermediate B3.

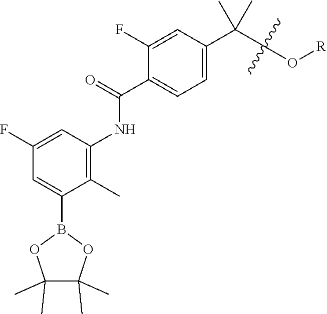

TABLE 5

| Intermediate | | |
|---|---|---|
| ID | Structure | Analytical Data-LCMS |
| B4 | 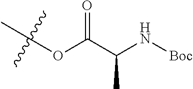 [SMILES: *OC(=O)[C@H](C)NC(=O)OC(C)(C)C] | LCMS Method T7: Rt = 1.40 min; MS m/z [M − H]⁻ = 601.5 |
| B5 | 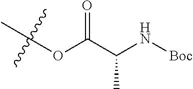 [SMILES: *OC(=O)[C@@H](C)NC(=O)OC(C)(C)C] | LCMS Method T7: Rt = 1.40 min; MS m/z [M − H]⁻ = 601.5 |
| B6 | 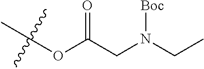 [SMILES: *OC(=O)CN(CC)C(=O)OC(C)(C)C] | LCMS Method T2: Rt = 1.58 min; MS m/z [M − H]⁻ = 615.5 |
| B7 | 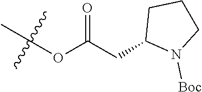 [SMILES: *OC(=O)C[C@@H]1CCCN1C(=O)OC(C)(C)C] | LCMS Method T7: Rt = 1.47 min, MS m/z [M + H]⁺ = 643.5 |
| B8 | 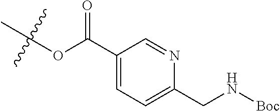 [SMILES: *OC(=O)c1ccc(CNC(=O)OC(C)(C)C)nc1] | LCMS Method T8: Rt = 3.44 min; MS m/z [M + H]⁺ = 666.9 |
| B9 | 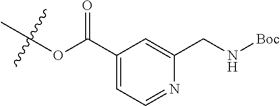 [SMILES: *OC(=O)c1ccnc(CNC(=O)OC(C)(C)C)c1] | LCMS Method T7: Rt = 1.35 min; MS m/z [M + H]⁺ = 666.5 |
| B10 | 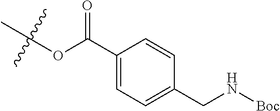 [SMILES: *OC(=O)c1ccc(CNC(=O)OC(C)(C)C)cc1] | LCMS Method T7: Rt = 1.45 min; MS m/z [M + H − Boc]⁺ = 565.5 |
| B11 | 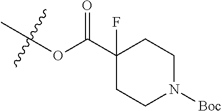 [SMILES: *OC(=O)C1(F)CCN(C(=O)OC(C)(C)C)CC1] | LCMS Method T2: Rt = 1.53 min; MS m/z [M − H]⁻ = 659.6 |
| B12 | 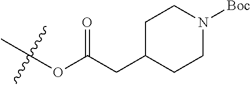 [SMILES: *OC(=O)CC1CCN(C(=O)OC(C)(C)C)CC1] | LCMS Method T7: Rt = 1.49 min; MS m/z [M + H − Boc]⁺ = 557.1 |
| B13 | 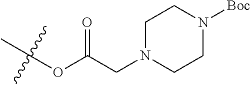 [SMILES: *OC(=O)CN1CCN(C(=O)OC(C)(C)C)CC1] | LCMS Method T2: Rt = 1.53 min; MS m/z [M + H]⁺ = 658.5 |
| B14 | 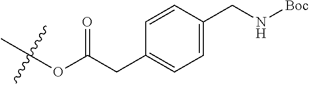 [SMILES: *OC(=O)Cc1ccc(CNC(=O)OC(C)(C)C)cc1] | LCMS Method T2: Rt = 1.57 min; MS m/z [M + H]⁺ = 679.5 |
| B15 | 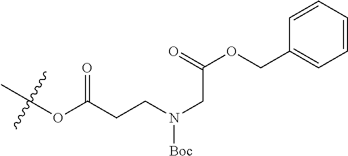 [SMILES: *OC(=O)CCN(CC(=O)OCc1ccccc1)C(=O)OC(C)(C)C] | LCMS Method W1: Rt = 8.12 min; MS m/z [M − H]⁻ = 749.5 |

### Example 4c: (3R,4S)-1-((5-Fluoro-2-methyl-3-(4,4,5,5-tetramethyl-1,3,2-dioxaborolan-2-yl)phenyl)carbamoyl)-4-isobutylpyrrolidin-3-yl (tert-butoxycarbonyl)glycinate (Intermediate B16)

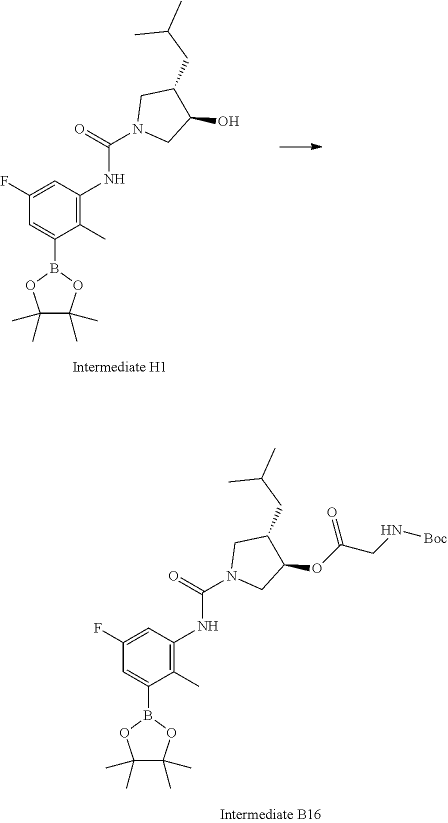 [SMILES: Cc1c(NC(=O)N2C[C@H](CC(C)C)[C@@H](O)C2)cc(F)cc1B1OC(C)(C)C(C)(C)O1 ; Cc1c(NC(=O)N2C[C@H](CC(C)C)[C@@H](OC(=O)CNC(=O)OC(C)(C)C)C2)cc(F)cc1B1OC(C)(C)C(C)(C)O1]

To a solution of (3R,4S)—N-(5-fluoro-2-methyl-3-(4,4,5,5-tetramethyl-1,3,2-dioxaborolan-2-yl)phenyl)-3-hydroxy-4-isobutylpyrrolidine-1-carboxamide (Intermediate H1, 840 mg, 1.998 mmol; preparation described in published PCT application WO2019/186358A1, intermediate 7) in DCM (20 mL) was added DCC (619 mg, 3.00 mmol), DMAP (317 mg, 2.60 mmol), and Boc-glycine (455 mg, 2.60 mmol). The RM was stirred at RT for 2 h under an atmosphere of Ar. The resulting precipitate was removed by filtration and washed with DCM. The filtrate was concentrated under reduced pressure and purified by chromatography on silica gel (RediSep® Rf, 80 g) eluting with EtOAc (from 0 to 100%) in heptane. The pure fractions were concentrated under reduced pressure to dryness to provide the title compound Intermediate B16 (690 mg) as a colorless solid. Method T2: Rt 1.38 min; MS m/z [M+H]⁺=578.4.

### Example 5c: Benzyl (2-((((2-(3-fluoro-4-((5-fluoro-2-methyl-3-(4,4,5,5-tetramethyl-1,3,2-dioxaborolan-2-yl)phenyl)carbamoyl)phenyl)propan-2-yl)oxy)(hydroxy)phosphoryl)oxy)ethyl)carbamate (Intermediate B17)

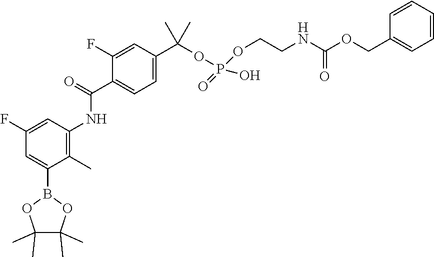 [SMILES: Cc1c(NC(=O)c2ccc(C(C)(C)OP(=O)(O)OCCNC(=O)OCc3ccccc3)cc2F)cc(F)cc1B1OC(C)(C)C(C)(C)O1]

### Step 1: 2-(3-Fluoro-4-((5-fluoro-2-methyl-3-(4,4,5,5-tetramethyl-1,3,2-dioxaborolan-2-yl)phenyl)carbamoyl)phenyl)propan-2-yl hydrogen phosphonate (Intermediate B17-1)

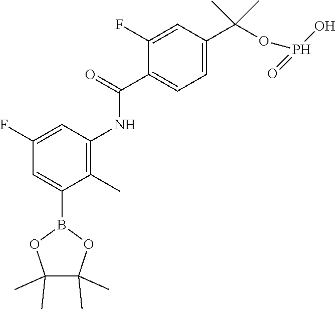

To a solution of 2-fluoro-N-(5-fluoro-2-methyl-3-(4,4,5,5-tetramethyl-1,3,2-dioxaborolan-2-yl)phenyl)-4-(2-hydroxypropan-2-yl)benzamide (Intermediate B1, 5 g, 11.59 mmol) in anhydrous THF (150 mL) was added pyridine (9.38 mL, 116 mmol) and diphenyl phosphite (3.49 mL, 23.19 mmol). The RM was stirred at RT for 16 h and then treated with 25% aq. ammonium hydroxide solution (18.06 mL, 116 mmol). Stirring was continued for 1 h at RT and then the volatiles removed under reduced pressure. The crude residue was triturated ultrasonically, and in a sequential manner, with Et₂O:heptane (1:1, 100 mL), EtOAc/heptane (1/1, 100 mL), warm ACN (50° C., 100 mL) and finally warm EtOAc (50° C., 100 mL). After the final trituration step, the remaining solid was dissolved in MeOH and concentrated under reduced pressure to give the title compound Intermediate B17-1 (6.025 g) as a white solid. LCMS Method T2: Rt=0.97 min; MS m/z [M+NH₄]+=513.2, [M−H]⁻=494.2.

### Step 2: Benzyl (2-((((2-(3-fluoro-4-((5-fluoro-2-methyl-3-(4,4,5,5-tetramethyl-1,3,2-dioxaborolan-2-yl)phenyl)carbamoyl)phenyl)propan-2-yl)oxy)(hydroxy)phosphoryl)oxy)ethyl)carbamate (Intermediate B17)

To a 100 mL round-button flask containing 2-(3-fluoro-4-((5-fluoro-2-methyl-3-(4,4,5,5-tetramethyl-1,3,2-dioxaborolan-2-yl)phenyl)carbamoyl)phenyl)propan-2-yl hydrogen phosphonate (Intermediate B17-1, 0.6008 g, 1.009 mmol) and benzyl N-(2-hydroxyethyl)carbamate (0.301 g, 1.513 mmol) was added anhydrous pyridine (20 mL) and a magnetic stir bar. The mixture was ultrasonicated until homogeneous and then concentrated under reduced pressure. The oily residue was left under high vacuum for 30 min at 50° C. and then re-dissolved in anhydrous THF (15 mL). After placing the stirring solution under an atmosphere of N₂, pyridine (0.816 mL, 10.09 mmol) and then pivaloyl chloride (0.186 mL, 1.513 mmol) were added. The RM was stirred at RT for 30 min and then cooled to 0° C. The mixture was then treated with a solution of iodine (0.256 g, 1.009 mmol) in pyridine (2 mL) and de-ionised water (2 mL). The RM was allowed to warm to RT and stirring continued for 2 h. The RM was diluted dropwise with an aq. sodium thiosulfate solution (20% w/v) until color of the solution disappeared. The RM was then concentrated under reduced pressure to give an off-white translucent solid. The crude material was re-dissolved in MeOH (70 mL) and pre-adsorbed onto Isolute® H-MN. Purification by chromatography on silica gel (RediSep® Rf, 40 g) eluting with MeOH in DCM (from 0 to 20%) gave a dark orange solid. The solid was triturated with EtOAc (100 mL) and then removed by filtration. The filter cake was washed with EtOAc (20 mL) and the filtrate concentrated under reduced pressure to give the title compound Intermediate B17 (0.418 g) as a dark orange wax-like solid. LCMS Method T2: Rt=1.08 min; MS m/z [M−H]⁻=687.4.

### Example 6c: tert-butyl (2-(((2-Fluoro-N-(5-fluoro-2-methyl-3-(4,4,5,5-tetramethyl-1,3,2-dioxaborolan-2-yl)phenyl)-4-(2-hydroxypropan-2-yl)benzamido)methyl)amino)-2-oxoethyl)carbamate (Intermediate B18)

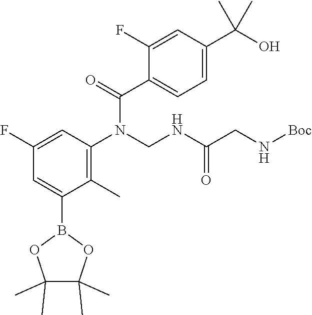 [SMILES: Cc1c(B2OC(C)(C)C(C)(C)O2)cc(F)cc1N(CNC(=O)CNC(=O)OC(C)(C)C)C(=O)c1ccc(C(C)(C)O)cc1F]

### Step 1: (2-((tert-Butoxycarbonyl)amino)acetamido)methyl acetate (Intermediate B18-1)

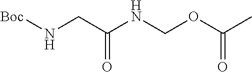 [SMILES: CC(=O)OCNC(=O)CNC(=O)OC(C)(C)C]

(tert-Butoxycarbonyl)glycylglycine (4.8242 g, 20.77 mmol) was dissolved in anhydrous THF (140 mL) and then concentrated under reduced pressure and left under high vacuum at 50° C. for 45 min. The solid was re-dissolved in anhydrous THF (100 mL) and Cu(OAc)₂ (0.377 g, 2.077 mmol) added. The stirring mixture was placed under N₂ atmosphere and then cooled to 0° C. Pb(OAc)₄ (13.82 g, 31.2 mmol) was added at once and the turquoise-colored RM was allowed to warm to RT and stirring continued for 2 h. The RM was filtered over sea-sand and the solids rinsed with THF (50 mL). The dark emerald-green filtrate was then concentrated under reduced pressure to give a turquoise-blue colored residue. Purification by chromatography on silica gel (RediSep® Rf, 80 g) eluting with EtOAc in heptane (from 0 to 100%) gave the title compound Intermediate B18-1 (2.855 g) as a clear, colorless oil. LCMS Method T2: Rt=0.64 min; m/z [M+H]⁺=247.1.

### Step 2: tert-Butyl (2-(((2-fluoro-N-(5-fluoro-2-methyl-3-(4,4,5,5-tetramethyl-1,3,2-dioxaborolan-2-yl)phenyl)-4-(2-hydroxypropan-2-yl)benzamido)methyl)amino)-2-oxoethyl)carbamate (Intermediate B18)

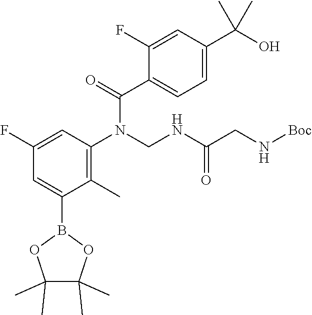 [SMILES: Cc1c(B2OC(C)(C)C(C)(C)O2)cc(F)cc1N(CNC(=O)CNC(=O)OC(C)(C)C)C(=O)c1ccc(C(C)(C)O)cc1F]

To a stirring solution of 2-fluoro-N-(5-fluoro-2-methyl-3-(4,4,5,5-tetramethyl-1,3,2-dioxaborolan-2-yl)phenyl)-4-(2-hydroxypropan-2-yl)benzamide (Intermediate B1, 2.1 g, 4.82 mmol) and (2-((tert-butoxycarbonyl)amino)acetamido)methyl acetate (Intermediate B18-1, 1.781 g, 7.23 mmol) in anhydrous ACN (60 mL) and under N₂ atmosphere was added K₂CO₃ (0.999 g, 7.23 mmol). The heterogeneous RM was stirred at RT for 72.5 h and then filtered. The filter cake was rinsed with ACN (40 mL) and DCM (40 mL) and the combined filtrates were concentrated under reduced pressure. Purification by chromatography on silica gel (RediSep® Rf, 40 g) eluting with EtOAc in heptane (from 0 to 90%) gave the title compound Intermediate B18 (1.764 g) as a white solid. LCMS Method T2: Rt=1.17 min; m/z [M+H]⁺=618.3.

### Intermediates V-A-P and V-A

### Method 1

### Example 1d: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 3-((tert-butoxycarbonyl)amino)propanoate (Intermediate V-A1-P)

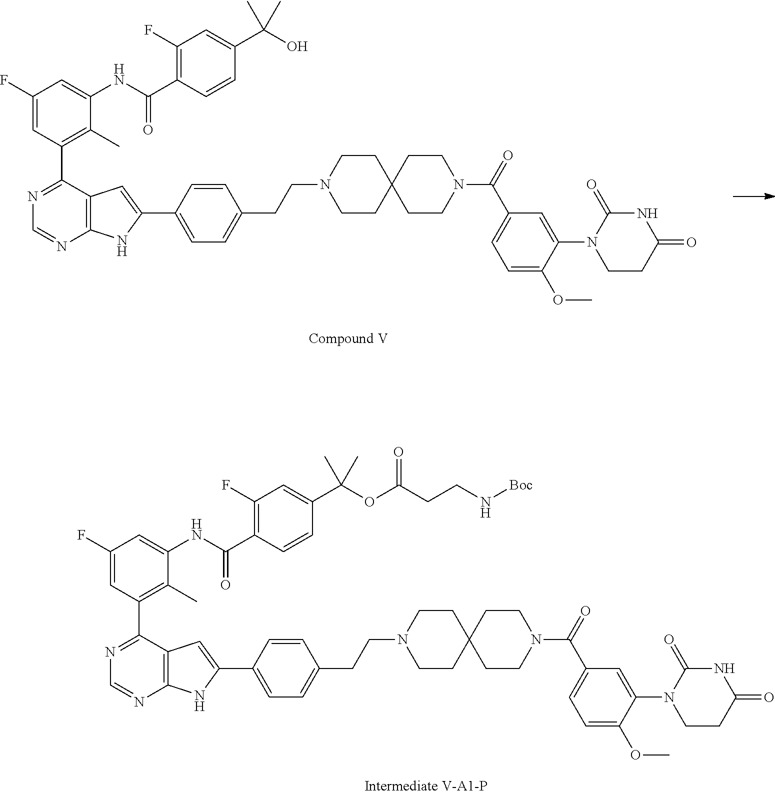 [SMILES: COc1ccc(C(=O)N2CCC3(CCN(CCc4ccc(-c5cc6c(-c7cc(F)cc(NC(=O)c8ccc(C(C)(C)O)cc8F)c7C)ncnc6[nH]5)cc4)CC3)CC2)cc1N1CCC(=O)NC1=O ; COc1ccc(C(=O)N2CCC3(CCN(CCc4ccc(-c5cc6c(-c7cc(F)cc(NC(=O)c8ccc(C(C)(C)OC(=O)CCNC(=O)OC(C)(C)C)cc8F)c7C)ncnc6[nH]5)cc4)CC3)CC2)cc1N1CCC(=O)NC1=O]

To a solution of N-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide (Compound V, 500 mg, 0.541 mmol) and 3-((tert-butoxycarbonyl)amino)propanoic acid (0.123 g, 0.649 mmol) in DCM (3.60 mL) was added DCC (0.223 g, 1.081 mmol) and DMAP (0.132 g, 1.081 mmol). The suspension was stirred at RT for 18 h. Additional 3-((tert-butoxycarbonyl)amino)propanoic acid (0.123 g, 0.649 mmol), DCC (0.223 g, 1.081 mmol) and DMAP (0.132 g, 1.081 mmol) was added and the RM was stirred for 24 h at RT. The RM was filtered, rinsed with a small amount of DCM and the filtrate concentrated under reduced pressure. The resulting residue was purified by column chromatography over silica gel (80 g, eluting with MeOH/DCM 0-20%) providing enriched material (688 mg) as a cream foam. To a solution of this material in THF (6.4 mL) was added pyridine (0.129 mL, 1.60 mmol) followed by anhydrous hydrazine solution in THF (1M, 2.56 mL). The solution was stirred at RT for 2 h. The RM was diluted with EtOAc (50 mL) and washed sat. aq. NaHCO₃ (2×25 mL). The separated organic layer was dried over Na₂SO₄, filtered and concentrated under reduced pressure. The resulting residue was dissolved in MeOH and purified by RP column chromatography (150 g C18Aq Gold column, eluting with ACN/water 10 to 40% with 0.1% NH₄OH modifier) providing the title compound Intermediate V-A1-P (325 mg) as a white solid. LCMS Method T9: Rt 1.15 min; MS m/z [M+H]⁺=1096.8.

### Method 2

### Example 2d: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl N-(tert-butoxycarbonyl)-N-ethylglycinate (Intermediate V-A2-P)

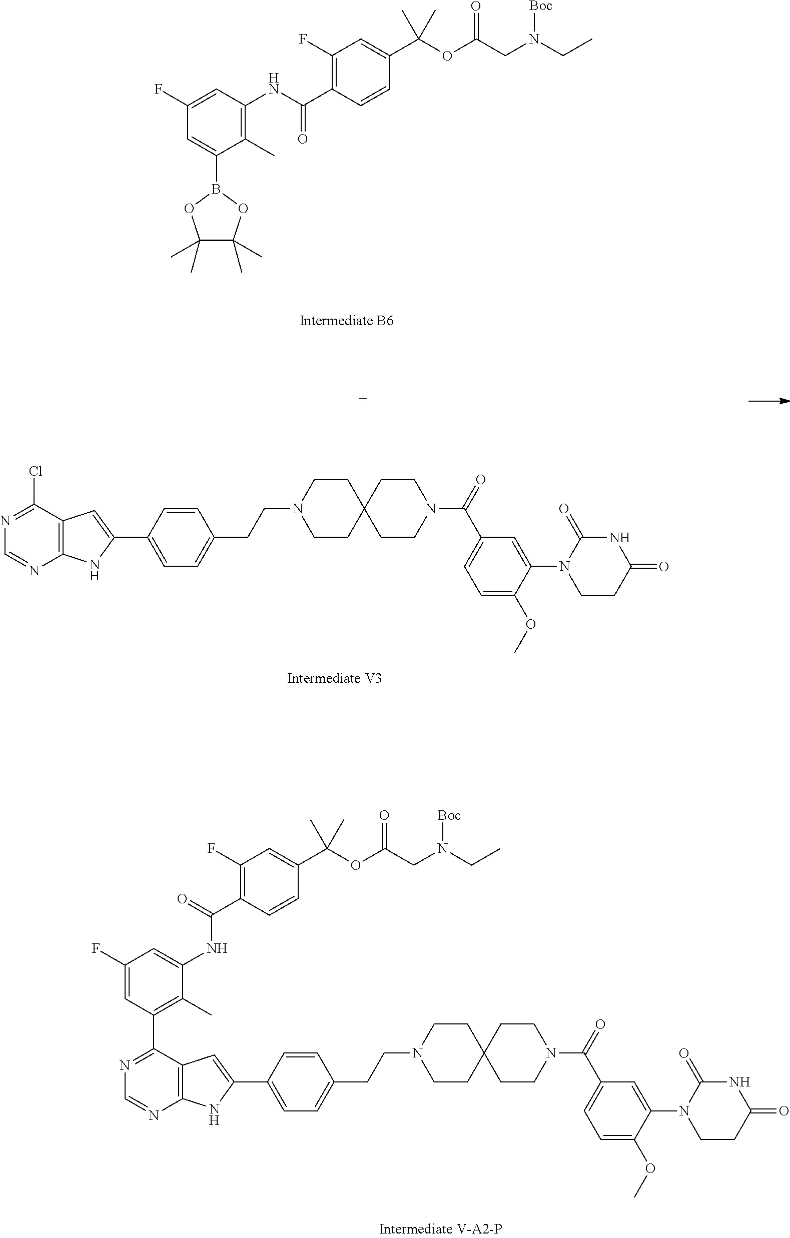 [SMILES: CCN(CC(=O)OC(C)(C)c1ccc(C(=O)Nc2cc(F)cc(B3OC(C)(C)C(C)(C)O3)c2C)c(F)c1)C(=O)OC(C)(C)C ; COc1ccc(C(=O)N2CCC3(CCN(CCc4ccc(-c5cc6c(Cl)ncnc6[nH]5)cc4)CC3)CC2)cc1N1CCC(=O)NC1=O ; CCN(CC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)OC(C)(C)C]

To solution of 1-(5-(9-(4-(4-Chloro-7H-pyrrolo[2,3-d]pyrimidin-6-yl)phenethyl)-3,9-diazaspiro[5.5]undecane-3-carbonyl)-2-methoxyphenyl)dihydropyrimidine-2,4(1H, 3H)-dione (Intermediate V3, 700 mg, 1.07 mmol) and 2-(3-fluoro-4-((5-fluoro-2-methyl-3-(4,4,5,5-tetramethyl-1,3,2-dioxaborolan-2-yl)phenyl)carbamoyl)phenyl)propan-2-yl N-(tert-butoxycarbonyl)-N-ethylglycinate (Intermediate B6, 858 mg, 1.28 mmol) in dioxane (3 mL) and water (3 mL) was added aq. Na₂CO₃ solution (2M, 1.33 mL, 2.67 mmol). The RM was purged with Ar and PdCl₂(dppf)-CH₂Cl₂ adduct (87 mg, 0.11 mmol) added. The RM was stirred at 80° C. for 5 min and then diluted with EtOAc. The organic layer was washed with water, dried over MgSO₄, filtered and concentrated under reduced pressure. The crude compound was purified by chromatography on silica gel (RediSep® Rf, 80 g) eluting with MeOH in DCM (0 to 20%). The obtained solid was dissolved in a mixture of MeOH/THF 4:1 and filtered through Agilent Stratospheres PL-Thiol 3 cartridges (3×500 mg/5 mL). The cartridges were washed with MeOH/THF (4/1, 10 mL) and the filtrate passed-through additional PL-Thiol cartridges (2×500 mg). The cartridges were washed with 10 mL of MeOH/THF 4:1 and the filtrate concentrated under reduced pressure to dryness to give the title compound (Intermediate V-A2-P, 686 mg). LCMS Method T2: Rt=1.06 min; MS m/z [M+H]⁺=1111.6. Alternatively, the RM can be stirred at 80 to 90° C. for 5 to 45 min.

### Method 3

### Example 3d: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 3-((tert-butoxycarbonyl)amino)-2,2-dimethylpropanoate (Intermediate V-A3-P)

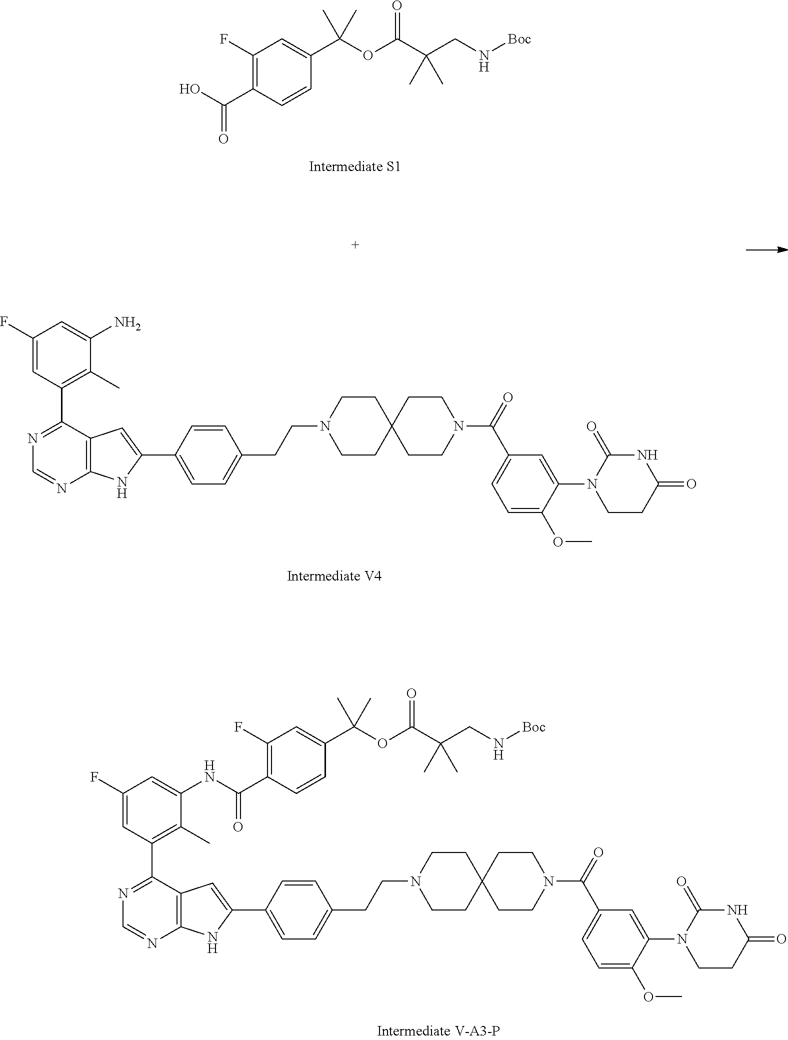 [SMILES: CC(C)(C)OC(=O)NCC(C)(C)C(=O)OC(C)(C)c1ccc(C(=O)O)c(F)c1 ; COc1ccc(C(=O)N2CCC3(CCN(CCc4ccc(-c5cc6c(-c7cc(F)cc(N)c7C)ncnc6[nH]5)cc4)CC3)CC2)cc1N1CCC(=O)NC1=O ; COc1ccc(C(=O)N2CCC3(CCN(CCc4ccc(-c5cc6c(-c7cc(F)cc(NC(=O)c8ccc(C(C)(C)OC(=O)C(C)(C)CNC(=O)OC(C)(C)C)cc8F)c7C)ncnc6[nH]5)cc4)CC3)CC2)cc1N1CCC(=O)NC1=O]

4-(2-((3-((tert-butoxycarbonyl)amino)-2,2-dimethylpropanoyl)oxy)propan-2-yl)-2-fluorobenzoic acid (Intermediate S1, 301 mg, 0.757 mmol) and HATU (597 mg, 1.571 mmol) were dissolved in DMF (2.52 mL) and DIPEA (331 μL, 1.893 mmol) was added under N₂ atmosphere. The RM was allowed to stir for ˜5 min and 1-(5-(9-(4-(4-(3-amino-5-fluoro-2-methylphenyl)-7H-pyrrolo[2,3-d]pyrimidin-6-yl)phenethyl)-3,9-diazaspiro[5.5]undecane-3-carbonyl)-2-methoxyphenyl)dihydropyrimidine-2,4(1H,3H)-dione (Intermediate V4, 282 mg, 0.379 mmol) was added in one portion. DMAP (40 mg, 0.327 mmol) was then added and the RM stirred at 50° C. for ˜18 h. The RM was cooled to RT and partitioned between water (75 mL) and EtOAc (150 mL). The separated organic layer was washed with water (75 mL) and the combined aq. layers re-extracted with EtOAc (75 mL). The combined organic layers were dried over Na₂SO₄, filtered and concentrated under reduced pressure. The resulting residue was purified by column chromatography (SiO₂, 40 g, eluting with MeOH/DCM=0/100 to 40/60) to provide the title compound (Intermediate V-A3-P, 200 mg) as a cream solid. LCMS Method T9: Rt=1.20 min; MS m/z [M+H]⁺=1124.7.

### Method 4

### Example 4d: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 3-(3-((tert-butoxycarbonyl)amino)propanamido)propanoate (Intermediate V-A4-P)

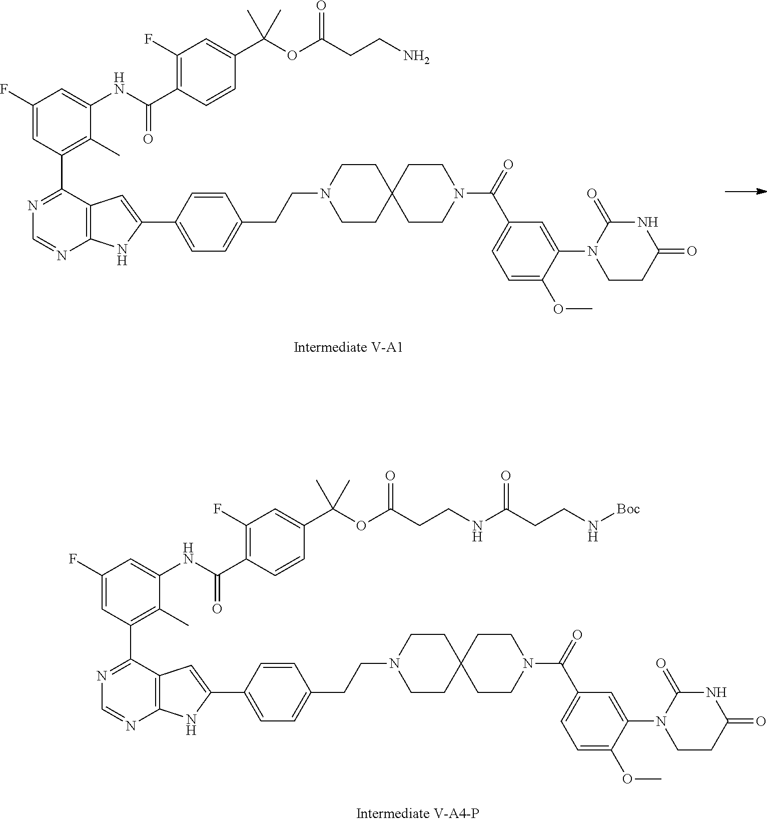 [SMILES: COc1ccc(C(=O)N2CCC3(CCN(CCc4ccc(-c5cc6c(-c7cc(F)cc(NC(=O)c8ccc(C(C)(C)OC(=O)CCN)cc8F)c7C)ncnc6[nH]5)cc4)CC3)CC2)cc1N1CCC(=O)NC1=O ; COc1ccc(C(=O)N2CCC3(CCN(CCc4ccc(-c5cc6c(-c7cc(F)cc(NC(=O)c8ccc(C(C)(C)OC(=O)CCNC(=O)CCNC(=O)OC(C)(C)C)cc8F)c7C)ncnc6[nH]5)cc4)CC3)CC2)cc1N1CCC(=O)NC1=O]

To crude 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 3-aminopropanoate (Intermediate V-A1, 401 mg, 0.3 mmol) in DCM (2 mL) was added 3-((tert-butoxycarbonyl)amino)propanoic acid (0.102 g, 0.540 mmol) and HATU (0.228 g, 0.600 mmol) followed by Huenig's Base (0.314 mL, 1.80 mmol). The mixture-under N₂ atmosphere in a sealed vial—was stirred until the mixture became a solution and stirring was continued for ˜20 min. The RM was diluted with DCM (˜8 mL) and washed twice with sat. aq. NaHCO₃ solution. The organic layer was dried over Na₂SO₄, filtered and concentrated under reduced pressure to approximately 1-2 mL. The residue was purified by column chromatography (40 g SiO₂, eluting with DCM/MeOH, 100/0 to 80/20) providing enriched title compound (Intermediate V-A4-P, 443 mg) which was directly used in the next step without further purification. LCMS Method T9: Rt=1.08 min; MS m/z [M+H]⁺=1167.8.

The following compounds in Table 6 were prepared, using the appropriate starting material, according to the procedures described for Examples 1d-4d, e.g., Methods 1-4, above.

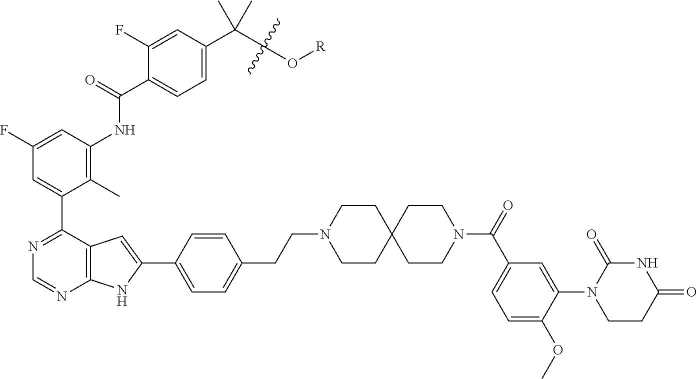

TABLE 6

| Intermediate | Method | Structure | Analytical-LCMS Method |
|---|---|---|---|
| V-A5-P | 2 | 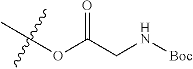 [SMILES: *OC(=O)CNC(=O)OC(C)(C)C] | LCMS Method W6: Rt = 0.98 min; MS m/z [M + H]⁺ = 1082.8 |
| V-A6-P | 2 | 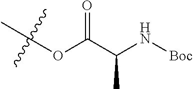 [SMILES: *OC(=O)[C@H](C)NC(=O)OC(C)(C)C] | LCMS Method T7: Rt = 0.92 min; MS m/z [M − H]⁻ = 1094.7 |
| V-A7-P | 2 | 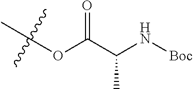 [SMILES: *OC(=O)[C@@H](C)NC(=O)OC(C)(C)C] | LCMS Method T7: Rt = 1.00 min; MS m/z [M − H]⁻ = 1094.8 |
| V-A8-P | 1 | 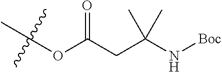 [SMILES: *OC(=O)CC(C)(C)NC(=O)OC(C)(C)C] | LCMS Method T9: Rt = 1.21 min; MS m/z [M + H]+ = 1125.2 |
| V-A9-P | 1 | 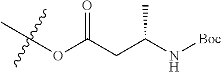 [SMILES: *OC(=O)C[C@H](C)NC(=O)OC(C)(C)C] | LCMS Method T9: Rt = 1.16 min; MS m/z [M + H]⁺ = 1112.0 |
| V-A10-P | 2 | 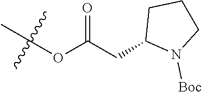 [SMILES: *OC(=O)C[C@@H]1CCCN1C(=O)OC(C)(C)C] | LCMS Method T9: Rt = 1.23 min, MS m/z [M + H]⁺ = 1138.0 |
| V-A11-P | 1 | 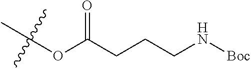 [SMILES: *OC(=O)CCCNC(=O)OC(C)(C)C] | LCMS Method T8: Rt = 2.09 min; MS m/z [M − H]⁻ = 1109 |
| V-A12-P | 2 | 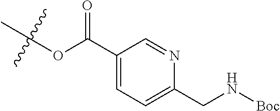 [SMILES: *OC(=O)c1ccc(CNC(=O)OC(C)(C)C)nc1] | LCMS Method T8: Rt = 2.08 min; MS m/z [M − H]⁻ = 1157.8 |
| V-A13-P | 2 | 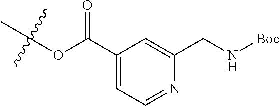 [SMILES: *OC(=O)c1ccnc(CNC(=O)OC(C)(C)C)c1] | LCMS Method T7: Rt = 0.91 min; MS m/z [M − H]⁻ = 1157.8 |
| V-A14-P | 2 | 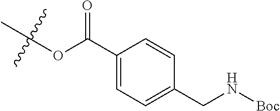 [SMILES: *OC(=O)c1ccc(CNC(=O)OC(C)(C)C)cc1] | LCMS Method T7: Rt = 0.98 min; MS m/z [M + H]⁺ = 1159.7 |
| V-A15-P | 2 | 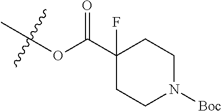 [SMILES: *OC(=O)C1(F)CCN(C(=O)OC(C)(C)C)CC1] | LCMS Method T2: Rt = 1.05 min; MS m/z [M − H]⁻ = 1152.7]⁻ |
| V-A16-P | 2 | 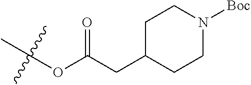 [SMILES: *OC(=O)CC1CCN(C(=O)OC(C)(C)C)CC1] | LCMS Method T7: Rt = 0.97 min; MS m/z [M + H]⁺ = 1150.5 |
| V-A17-P | 2 | 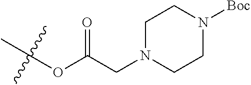 [SMILES: *OC(=O)CN1CCN(C(=O)OC(C)(C)C)CC1] | LCMS Method T2: Rt = 0.97 min; MS m/z [M − H]⁻ = 1149.7 |
| V-A18-P | 2 | 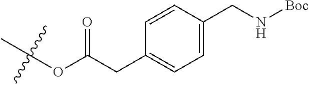 [SMILES: *OC(=O)Cc1ccc(CNC(=O)OC(C)(C)C)cc1] | LCMS Method T2: Rt = 1.08 min; MS m/z [M − H]⁻ = 1170.8 |
| V-A19-P | 2 | 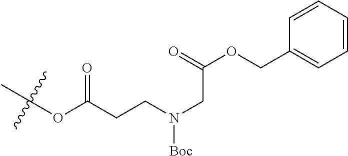 [SMILES: *OC(=O)CCN(CC(=O)OCc1ccccc1)C(=O)OC(C)(C)C] | LCMS Method W5: Rt = 1.05 min; MS m/z [M + 2H]²⁺ = 623.1 |

### Example 5d: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 3-aminopropanoate (Intermediate V-A1)

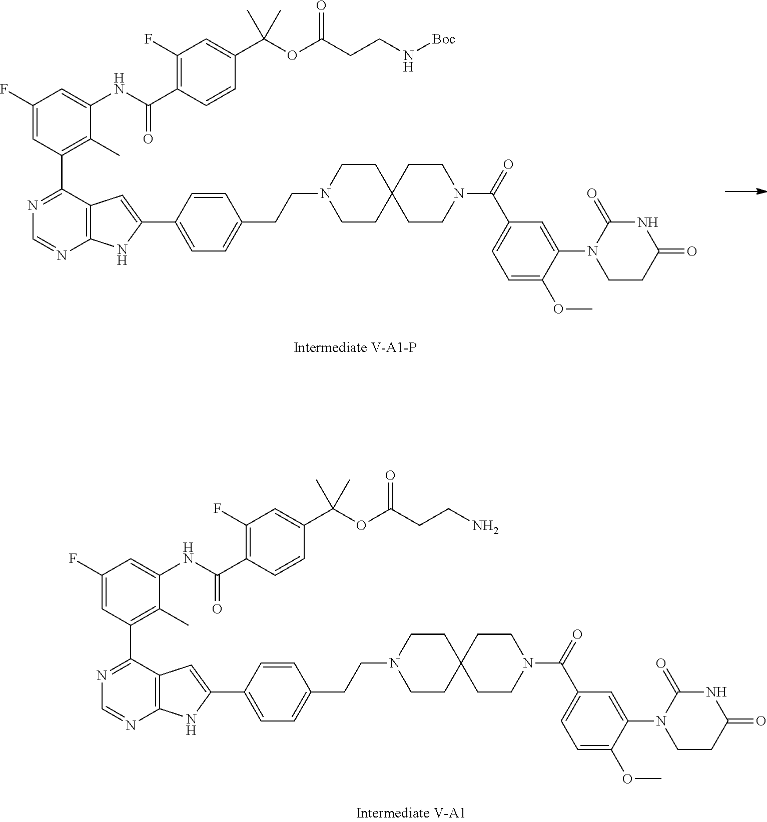 [SMILES: COc1ccc(C(=O)N2CCC3(CCN(CCc4ccc(-c5cc6c(-c7cc(F)cc(NC(=O)c8ccc(C(C)(C)OC(=O)CCNC(=O)OC(C)(C)C)cc8F)c7C)ncnc6[nH]5)cc4)CC3)CC2)cc1N1CCC(=O)NC1=O ; COc1ccc(C(=O)N2CCC3(CCN(CCc4ccc(-c5cc6c(-c7cc(F)cc(NC(=O)c8ccc(C(C)(C)OC(=O)CCN)cc8F)c7C)ncnc6[nH]5)cc4)CC3)CC2)cc1N1CCC(=O)NC1=O]

To a stirring solution of 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 3-((tert-butoxycarbonyl)amino)propanoate (Intermediate V-A1-P, 325 mg, 0.296 mmol) in DCM (3 mL) in a sealed vial and under N₂ atmosphere was added slowly TFA (914 uL, 11.9 mmol) at 0° C. Stirring was continued for ˜20 min and the cold mixture was concentrated under reduced pressure at RT. To the residue was added heptane (˜5 mL) and the mixture was agitated for ˜30 sec and then ultrasonicated for ˜30 sec. The heptane layer was decanted-off and the treatment repeated with additional heptane (˜5 mL). The remaining residue was dried under high vacuum for ˜2¼ h providing the crude title compound Intermediate V-A1, which was directly used in next reaction without further purification. LCMS Method T9: Rt=1.02 min; MS m/z [M+H]⁺=996.8. Alternatively, the RM can be stirred at 0° C. to RT 10 min to 6 h.

The following compounds in Table 7 were prepared using the appropriate starting material, according to the procedure described for Intermediate V-A1, in Example 5d above.

TABLE 7

| Intermediate | | |
|---|---|---|
| ID | Structure | Analytical-LCMS Method |
| V-A2 | 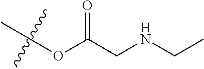 [SMILES: *OC(=O)CNCC] | LCMS Method T2: Rt = 0.70 min; MS m/z [M + H]⁺ = 1010.7 |
| V-A3 | 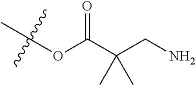 [SMILES: *OC(=O)C(C)(C)CN] | LCMS Method T9: Rt = 1.06 min; MS m/z [M + H]⁺ = 1025.1 |
| V-A4 | 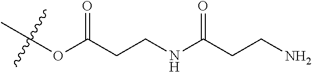 [SMILES: *OC(=O)CCNC(=O)CCN] | LCMS Method T9: Rt = 1.00 min; MS m/z [M + H]⁺ = 1067.7 |
| V-A5 | 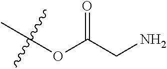 [SMILES: *OC(=O)CN] | LCMS Method W6: Rt = 0.67 min; MS m/z [M + H]⁺ = 982.7 |
| V-A6 | 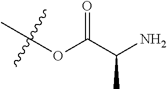 [SMILES: *OC(=O)[C@H](C)N] | LCMS Method T7: Rt = 0.70 min; MS m/z [M − H]⁻ = 994.7 |
| V-A7 | 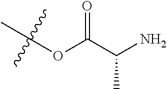 [SMILES: *OC(=O)[C@@H](C)N] | LCMS Method T7: Rt = 0.75 min; MS m/z [M − H]⁻ = 994.7 |
| V-A8 | 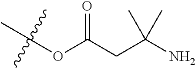 [SMILES: *OC(=O)CC(C)(C)N] | LCMS Method T9: Rt = 1.03 min, MS m/z [M + H]⁺ = 1024.7 |
| V-A9 | 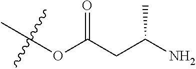 [SMILES: *OC(=O)C[C@H](C)N] | LCMS Method T9: Rt = 1.04 min, MS m/z [M + H]⁺ = 1011.1 |
| V-A10 | 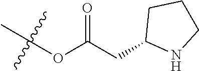 [SMILES: *OC(=O)C[C@@H]1CCCN1] | LCMS Method T9: Rt = 1.15 min, MS m/z [M + H]⁺ = 1037.1 |
| V-A11 | 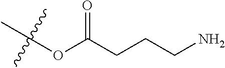 [SMILES: *OC(=O)CCCN] | LCMS Method T7: Rt = 0.80 min; MS m/z [M − H]⁻ = 1009 |
| V-A12 | 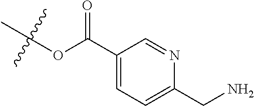 [SMILES: *OC(=O)c1ccc(CN)nc1] | LCMS Method T7: Rt = 0.85 min; MS m/z [M − H]⁻ = 1057.8 |
| V-A13 | 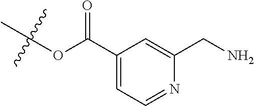 [SMILES: *OC(=O)c1ccnc(CN)c1] | LCMS Method T7: Rt = 0.71 min; MS m/z [M − H]⁻ = 1057.6 |
| V-A14 | 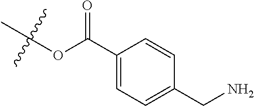 [SMILES: *OC(=O)c1ccc(CN)cc1] | LCMS Method T7: Rt = 0.69 min; MS m/z [M + H]⁺ = 1059.3 |
| V-A15 | 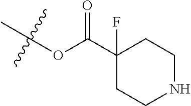 [SMILES: *OC(=O)C1(F)CCNCC1] | LCMS Method T2: Rt = 0.68 min; MS m/z [M + H]⁺ = 1054.8 |
| V-A16 | 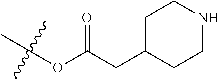 [SMILES: *OC(=O)CC1CCNCC1] | LCMS Method T7: Rt = 0.72 min; MS m/z [M + H]⁺ = 1050.8 |
| V-A17 | 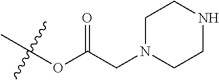 [SMILES: *OC(=O)CN1CCNCC1] | LCMS Method T2: Rt = 0.72 min; MS m/z [M − H]⁻ = 1049.9 |
| V-A18 | 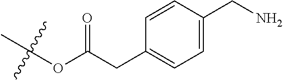 [SMILES: *OC(=O)Cc1ccc(CN)cc1] | LCMS Method T2: Rt = 0.76 min; MS m/z [M − H]⁻ = 1070.8 |
| V-A19 | 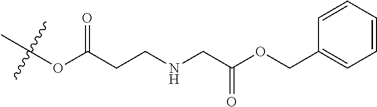 [SMILES: *OC(=O)CCNCC(=O)OCc1ccccc1] | LCMS Method W5: Rt = 0.80 min; MS m/z [M + H]⁺ = 1144.8 |

### Example 6d: (3R,4S)-1-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-4-isobutylpyrrolidin-3-yl (tert-butoxycarbonyl)glycinate (Intermediate H-A1-P)

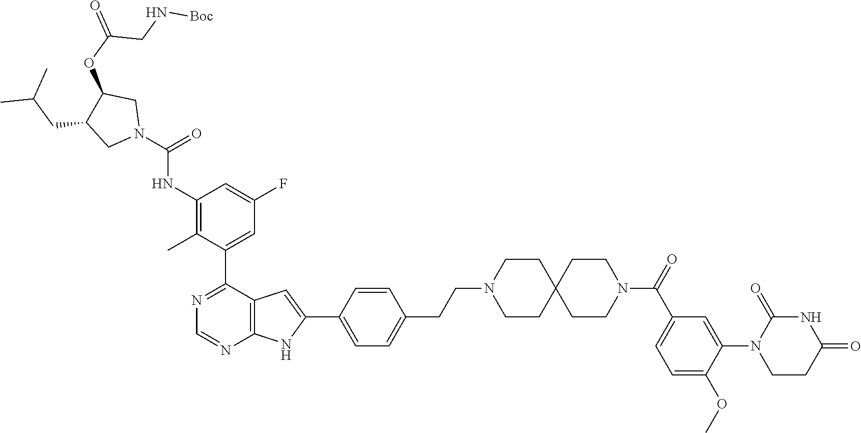 [SMILES: COc1ccc(C(=O)N2CCC3(CCN(CCc4ccc(-c5cc6c(-c7cc(F)cc(NC(=O)N8C[C@H](CC(C)C)[C@@H](OC(=O)CNC(=O)OC(C)(C)C)C8)c7C)ncnc6[nH]5)cc4)CC3)CC2)cc1N1CCC(=O)NC1=O]

Intermediate H-A1-P was prepared from Intermediate B16 and Intermediate V3 according to the Pd-mediated coupling procedure described for Intermediate V-A2-P (Method 2 in Example 2d) above. LCMS Method T2: Rt 0.92 min; MS m/z [M+H]⁺=1071.9.

### Example 7d: (3R,4S)-1-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-4-isobutylpyrrolidin-3-yl glycinate (Intermediate H-A1)

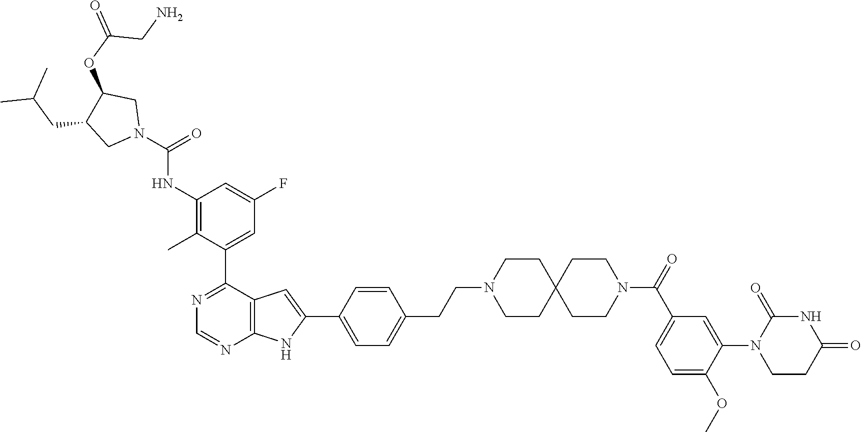 [SMILES: COc1ccc(C(=O)N2CCC3(CCN(CCc4ccc(-c5cc6c(-c7cc(F)cc(NC(=O)N8C[C@H](CC(C)C)[C@@H](OC(=O)CN)C8)c7C)ncnc6[nH]5)cc4)CC3)CC2)cc1N1CCC(=O)NC1=O]

Intermediate H-A1 was prepared from Intermediate H-A1-P according to the procedure described for Intermediate V-A1 in Example 5d. LCMS Method T2: Rt 0.67 min; MS m/z [M+H]⁺=971.9.

### Example 8d: Benzyl (2-((((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)(hydroxy)phosphoryl)oxy)ethyl)carbamate (Intermediate P-A1-P)

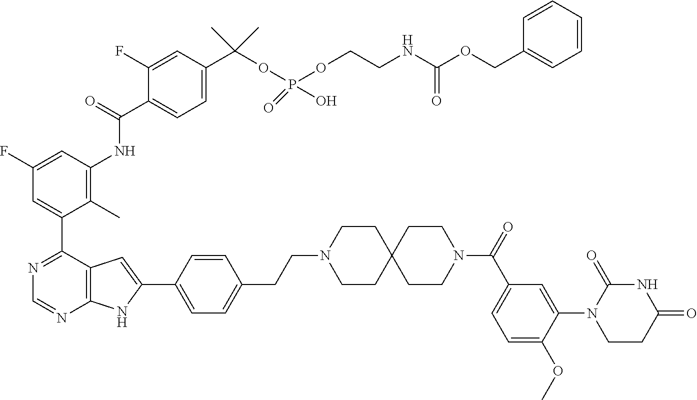 [SMILES: COc1ccc(C(=O)N2CCC3(CCN(CCc4ccc(-c5cc6c(-c7cc(F)cc(NC(=O)c8ccc(C(C)(C)OP(=O)(O)OCCNC(=O)OCc9ccccc9)cc8F)c7C)ncnc6[nH]5)cc4)CC3)CC2)cc1N1CCC(=O)NC1=O]

Intermediate P-A1-P was prepared from Intermediate B17 and Intermediate V3 according to the Pd-mediated coupling procedure described for Intermediate V-A2-P (Method 2 in Example 2d), above, heating at 80° C. for 30 min. LCMS Method T2: Rt=0.79 min; MS m/z [M−H]⁻=1180.5.

### Example 9d: 2-Aminoethyl (2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl) hydrogen phosphate (Intermediate P-A1)

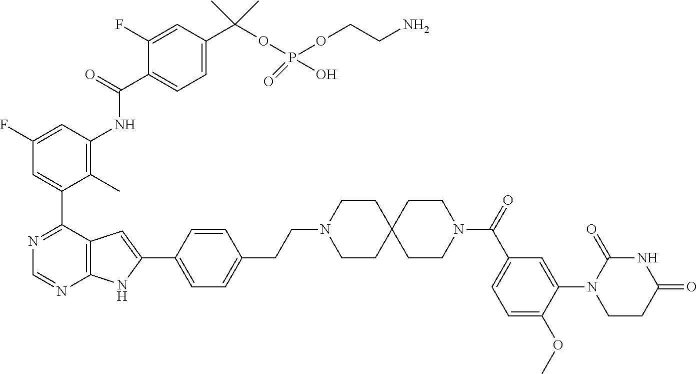 [SMILES: COc1ccc(C(=O)N2CCC3(CCN(CCc4ccc(-c5cc6c(-c7cc(F)cc(NC(=O)c8ccc(C(C)(C)OP(=O)(O)OCCN)cc8F)c7C)ncnc6[nH]5)cc4)CC3)CC2)cc1N1CCC(=O)NC1=O]

A suspension of benzyl (2-((((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)(hydroxy)phosphoryl)oxy)ethyl)carbamate (Intermediate P-A1-P, 334 mg, 0.246 mmol) and Pd/C [BASF 4505 D/R E] (288 mg, 0.270 mmol, 10 wt. %) in MeOH/THF (3/1, 20 mL) was agitated under a 0.1 bar H₂ atmosphere for ˜22 h at RT. The mixture was then filtered and the collected solids were washed with water/MeOH (1/4, 50 mL). The filtrate was partially concentrated under reduced pressure until ˜30 mL of solution remained. This solution was passed sequentially through two Agilent Stratospheres PL-Thiol MP cartridges (2×5 g). The cartridges were then washed with water/MeOH (1/4, 3×50 mL) and the combined filtrates concentrated under reduced pressure to give the title compound Intermediate P-A1 (198 mg) as a beige solid. LCMS Method T2: Rt=0.64 min; MS m/z [M−H]⁻=1046.6.

### Example 10d: tert-butyl (2-(((N-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamido)methyl)amino)-2-oxoethyl)carbamate

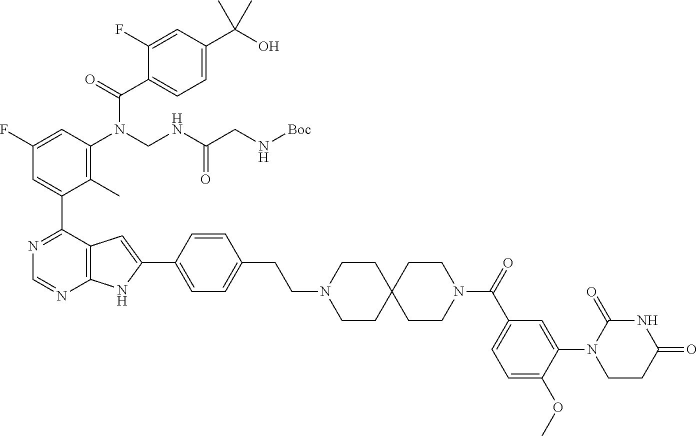 [SMILES: COc1ccc(C(=O)N2CCC3(CCN(CCc4ccc(-c5cc6c(-c7cc(F)cc(N(CNC(=O)CNC(=O)OC(C)(C)C)C(=O)c8ccc(C(C)(C)O)cc8F)c7C)ncnc6[nH]5)cc4)CC3)CC2)cc1N1CCC(=O)NC1=O]

Intermediate M-A1-P was prepared from Intermediate B18 and Intermediate V3 according to the Pd-mediated coupling procedure described for Intermediate V-A2-P in Example 2d (Method 2) above, heating at 80° C. for 15 min. LCMS Method T2: Rt=0.80 min; m/z [M−H]⁻=1109.5.

### Example 11d: N-((2-Aminoacetamido)methyl)-N-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide (Intermediate M-A1)

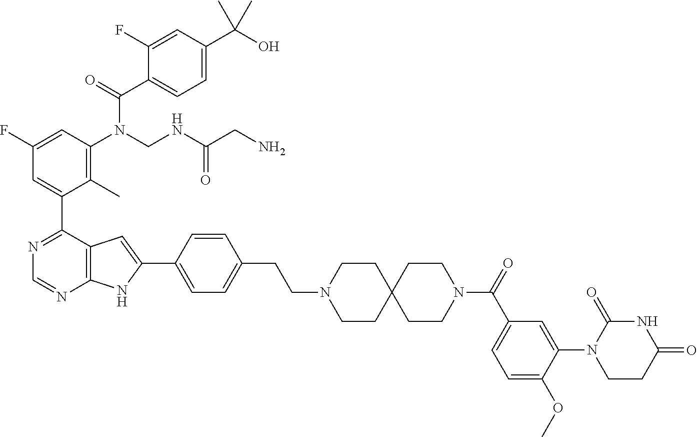 [SMILES: COc1ccc(C(=O)N2CCC3(CCN(CCc4ccc(-c5cc6c(-c7cc(F)cc(N(CNC(=O)CN)C(=O)c8ccc(C(C)(C)O)cc8F)c7C)ncnc6[nH]5)cc4)CC3)CC2)cc1N1CCC(=O)NC1=O]

To a light suspension of tert-butyl (2-(((N-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamido)methyl)amino)-2-oxoethyl)carbamate (Intermediate M-A1-P, 1.195 g, 1.022 mmol) in ACN (10 mL) and de-ionised water (5 mL) was added 6M aq. HCl (1.703 mL). The RM was stirred at 55° C. for 8 h and then partitioned between EtOAc (100 mL) and water (100 mL). The separated aq. layer was slowly added to a stirring mixture of n-BuOH (100 mL) and sat. aq. NaHCO₃ (100 mL). The layers were separated and the aq. phase was re-extracted with n-BuOH (100 mL). The combined organic phases were washed with brine (100 mL), dried over MgSO₄, filtered and then concentrated under reduced pressure to give the title compound Intermediate M-A1 (1.087 g) as an off-white/beige solid. LCMS Method T2: Rt=0.59 min; m/z [M−H]⁻=1009.5.

### Example 12d: tert-Butyl (2-(((6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-4-(5-fluoro-3-(2-fluoro-4-(2-hydroxypropan-2-yl)benzamido)-2-methylphenyl)-7H-pyrrolo[2,3-d]pyrimidin-7-yl)methyl)amino)-2-oxoethyl)carbamate (Intermediate R-A1-P)

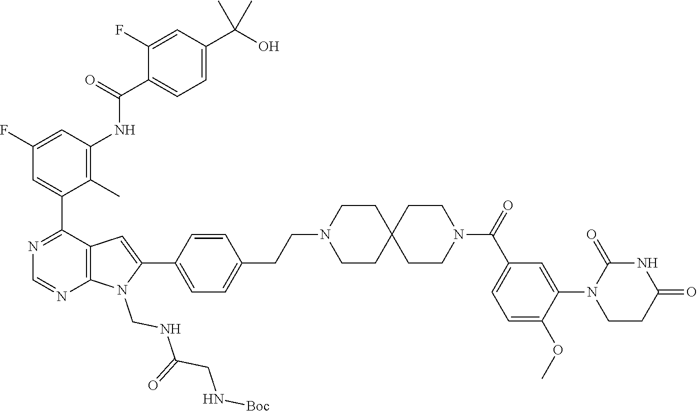 [SMILES: COc1ccc(C(=O)N2CCC3(CCN(CCc4ccc(-c5cc6c(-c7cc(F)cc(NC(=O)c8ccc(C(C)(C)O)cc8F)c7C)ncnc6n5CNC(=O)CNC(=O)OC(C)(C)C)cc4)CC3)CC2)cc1N1CCC(=O)NC1=O]

A stirring solution of N-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide (Compound V, 1.416 g, 1.516 mmol) and (2-((tert-butoxycarbonyl)amino)acetamido)methyl acetate (Intermediate B18-1, 0.972 g, 3.79 mmol) in anhydrous DMF (70 mL) was added Na₂CO₃ (0.803 g, 7.58 mmol) and the RM was stirred at RT for 16 h. De-ionised water (150 mL) was added and stirring was continued for 10 min. The solids were removed by filtration and the filter cake washed with de-ionised water (50 mL). The filter cake was dissolved in DCM (100 mL) and then concentrated under reduced pressure. Purification by chromatography on silica gel (RediSep® Rf, 80 g) eluting with MeOH in DCM from 0 to 20% gave enriched title compound Intermediate R-A1-P (1.704 g) as a bright yellow powder. LCMS Method W6: Rt=0.85/0.87 min; m/z [M−H]⁻=1109.6.

### Example 13d: N-(3-(7-((2-Aminoacetamido)methyl)-6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide (Intermediate R-A1)

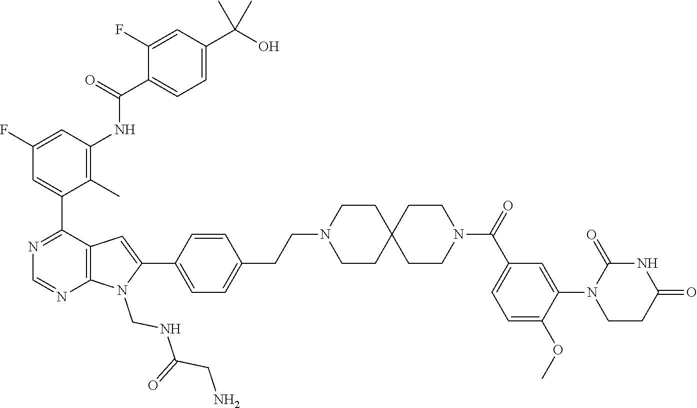 [SMILES: COc1ccc(C(=O)N2CCC3(CCN(CCc4ccc(-c5cc6c(-c7cc(F)cc(NC(=O)c8ccc(C(C)(C)O)cc8F)c7C)ncnc6n5CNC(=O)CN)cc4)CC3)CC2)cc1N1CCC(=O)NC1=O]

A stirring solution of tert-butyl (2-(((6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-4-(5-fluoro-3-(2-fluoro-4-(2-hydroxypropan-2-yl)benzamido)-2-methylphenyl)-7H-pyrrolo[2,3-d]pyrimidin-7-yl)methyl)amino)-2-oxoethyl)carbamate (Intermediate R-A1-P, 0.476 g, 0.368 mmol) and triisopropylsilane (0.091 mL, 0.442 mmol) in DCM (15 mL) was treated with TFA (1.42 mL, 18.4 mmol). After stirring at RT for 1 h, the RM was slowly added to a vigorously stirring mixture of DCM (100 mL) and sat. aq. NaHCO₃ (100 mL). The layers were separated and the aq. layer re-extracted with DCM (100 mL). The combined organic layers were washed with brine (100 mL), dried over MgSO₄, filtered and concentrated under reduced pressure providing the title compound Intermediate R-A1 (125 mg) as an off-white/beige solid. LCMS Method W6: Rt=0.70 min; m/z [M+H]⁺=1011.4, [M−H]⁻=1009.6.

### Intermediates V-A-PEG

### Example 14d: 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (2,2-dimethyl-4-oxo-3,8,11,14,17,20,23,26,29,32,35,38,41-tridecaoxa-5-azatetratetracontan-44-oyl)glycinate (Intermediate V-A5-Peg12-P)

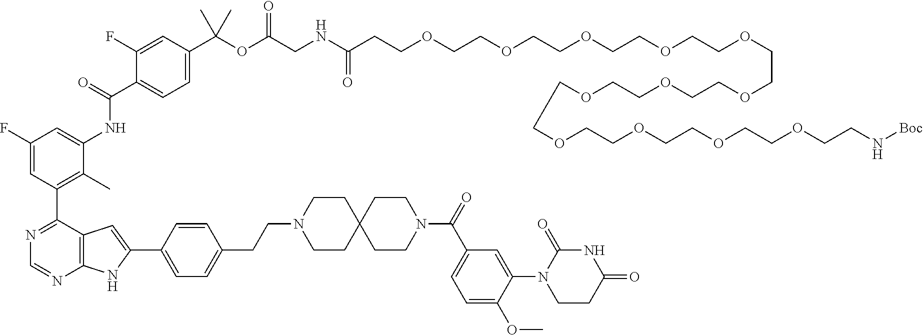 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCNC(=O)OC(C)(C)C)cc8F)c7C)ncnc6[nH]5)cc4)CC3)CC2)cc1N1CCC(=O)NC1=O]

To a solution of 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl glycinate TFA salt (Intermediate V-A5, 300 mg, 0.274 mmol) and DIPEA (0.48 mL) in DMF (1.5 mL) was added a solution of 2,2-dimethyl-4-oxo-3,8,11,14,17,20,23,26,29,32,35,38,41-tridecaoxa-5-azatetratetracontan-44-oic acid (216 mg, 0.301 mmol) and HATU (114 mg, 0.301 mmol) in DMF (1.5 mL). The RM was stirred at RT for 30 min and then the volatiles were removed with a stream of N2 for ˜3 h. The residue was diluted with DMSO (3.5 mL) and purified over a RP chromatography RediSep® Gold C18 column (130 g, 30 μm) eluting with ACN/water (0.1% TFA as modifier) with 5% ACN/water (0.1% TFA) for 5 min followed by 5 to 60% ACN/water (0.1% TFA) to provide, after lyophilization, the title compound Intermediate V-A5-Peg12-P (426 mg). LCMS Method T8: Rt=1.99 min; MS m/z [M+2H]²⁺=841.7.

### Example 15d: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (2,2-dimethyl-4-oxo-3,8,11,14,17,20,23,26,29,32,35,38,41,44,47,50,53,56,59,62,65,68,71,74,77-pentacosaoxa-5-azaoctacontan-80-oyl)glycinate (Intermediate V-A5-Peg24-P)

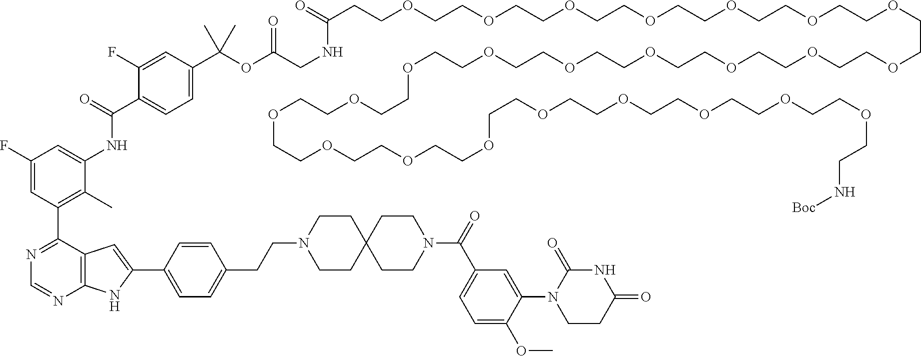 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCOCCOCCOCCOCCOCCOCCOCCOCCOCCOCCOCCOCCNC(=O)OC(C)(C)C)cc8F)c7C)ncnc6[nH]5)cc4)CC3)CC2)cc1N1CCC(=O)NC1=O]

Intermediate V-A5-Peg24-P was prepared according to the method described for Intermediate V-A5-Peg12-P in Example 12d herein above using Intermediate V-A5 and 2,2-dimethyl-4-oxo-3,8,11,14,17,20,23,26,29,32,35,38,41,44,47,50,53,56,59,62,65,68,71,74,77-pentacosaoxa-5-azaoctacontan-80-oic acid. LCMS Method T8: Rt=2.12 min; MS m/z [M+2H]²⁺=1106.3.

### Example 16d: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (1-amino-3,6,9,12,15,18,21,24,27,30,33,36-dodecaoxanonatriacontan-39-oyl)glycinate (Intermediate V-A5-Peg12)

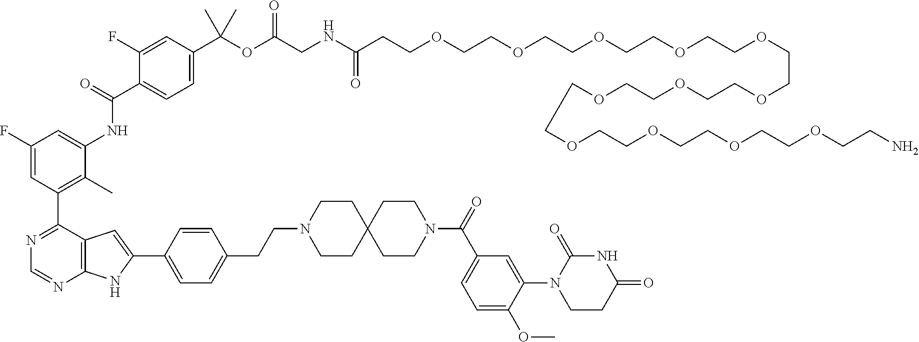 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCN)cc8F)c7C)ncnc6[nH]5)cc4)CC3)CC2)cc1N1CCC(=O)NC1=O]

To a solution of 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 2,2-dimethyl-4-oxo-3,8,11,14,17,20,23,26,29,32,35,38,41-tridecaoxa-5-azatetratetracontan-44-oyl)glycinate TFA salt (Intermediate V-A5-Peg12-P, 426 mg, 0.237 mmol) in DCM (10 mL) was added precooled (˜0° C.) TFA (1.17 mL) and the RM was allowed to stir at RT for 30 min. The mixture was diluted with DCE (10 mL) and concentrated under reduced pressure and the resulting residue dried under high vacuum for 16 h. Purification over a RP chromatography C18 column (Interchim Puriflash HQ, 120 g, spherical silica, 15 μm) with 5% ACN/water (0.1% TFA) for 5 min followed by 5 to 60% ACN/water (0.1% TFA) for 20 min provided after lyophilization the title compound Intermediate V-A5-Peg12 (280 mg) as its TFA salt. LCMS Method T8: Rt=1.55 min; MS m/z [M+2H]²⁺=791.9.

### Example 17d: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (1-amino-3,6,9,12,15,18,21,24,27,30,33,36,39,42,45,48,51,54,57,60,63,66,69,72-tetracosaoxapentaheptacontan-75-oyl)glycinate (Intermediate V-A5-Peg24)

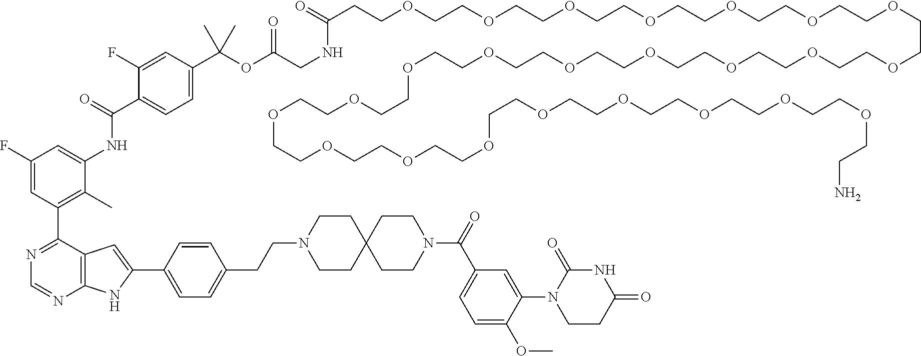 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCOCCOCCOCCOCCOCCOCCOCCOCCOCCOCCOCCOCCN)cc8F)c7C)ncnc6[nH]5)cc4)CC3)CC2)cc1N1CCC(=O)NC1=O]

Intermediate V-A5-Peg24 was prepared according to the method described for Intermediate V-A5-Peg12 in Example 14d herein above, using Intermediate V-A5-Peg24-P. LCMS Method T7: Rt=0.79 min; MS m/z [M+2H]²⁺=1056.3.

### Example 18d: 13-(Benzyloxy)-2-((benzyloxy)carbonyl)-13-oxo-2-undecyltridecanoic acid (Intermediate rac-F1)

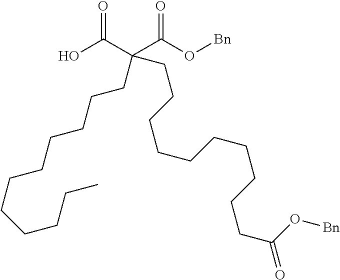 [SMILES: CCCCCCCCCCCC(CCCCCCCCCCC(=O)OCc1ccccc1)(C(=O)O)C(=O)OCc1ccccc1]

### Step 1: Benzyl 11-bromoundecanoate (Intermediate rac-F1-1)

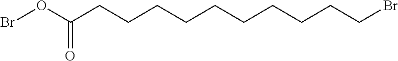 [SMILES: O=C(CCCCCCCCCCBr)OBr]

To a mixture of 11-bromoundecanoic acid (4.60 kg, 17.3 mol) in DCM (26.5 kg) was added EDCl (3.8 kg, 20.2 mol) in portions at 0° C. along with DMAP (98 g, 0.8 mol, 0.05 equiv). BnOH (1.70 kg, 15.7 mol) was then added dropwise. After stirring at 20° C. for 4 h, water (70.0 kg) was added slowly. The mixture was then concentrated under vacuum. Heptane (23.2 kg) and 19% NaCl solution (17 kg) were added for phase separation. The organic phase was washed 2× under basic conditions (5% Na₂CO₃, 25.0 kg; 19% NaCl solution, 25.0 kg), 1× under acidic conditions (5.2% HCl aq. solution, 25.0 kg; 19% NaCl solution, 25.0 kg), 1× with water (5.0 kg), and 1× with brine (5.0 kg). The organic layer was then concentrated under vacuum at 50° C. to provide the title compound Intermediate rac-F1-1 which was used in the next step without purification. ¹H NMR (400 MHz, Chloroform-d) δ [ppm] 1.18-1.36 (m, 10H), 1.37-1.47 (m, 2H), 1.64 (quin, J=7.33 Hz, 2H), 1.85 (dt, J=14.56, 7.06 Hz, 2H), 2.35 (t, J=7.58 Hz, 2H), 3.40 (t, J=6.88 Hz, 2H), 5.11 (s, 2H), 7.28-7.45 (m, 5H).

### Step 2: 1,11-Dibenzyl 11-(tert-butyl) docosane-1,11,11-tricarboxylate (Intermediate rac-F1-2)

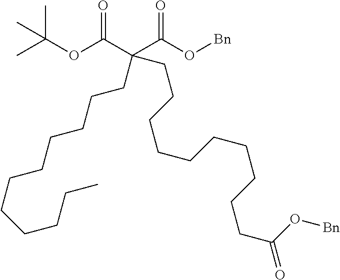 [SMILES: CCCCCCCCCCCC(CCCCCCCCCCC(=O)OCc1ccccc1)(C(=O)OCc1ccccc1)C(=O)OC(C)(C)C]

To a solution of benzyl tert-butyl malonate (3.0 kg, 12.0 mol) in NMP (30 L) was added 1-iodoundecane (3.55 kg, 12.58 mol) and Cs₂CO₃ (11.76 kg, 36.09 mol) at 20° C. The mixture was stirred at 20° C. for 6 h. To the mixture was then added benzyl 11-bromoundecanoate (Intermediate rac-F1-1, 5.53 kg, 15.6 mol) and the RM was heated to 80° C. and stirred for 12 h. The mixture was then cooled to 20° C. and a mixture of water (30 kg) and heptane (10 kg) was added. After stirring for 30 min, the organic layer was separated and washed 3× with the mixture of brine (5 kg) and MeOH (4 kg). The organic layer was dried over Na₂SO₄ and concentrated under reduced pressure. Further purification was done by column chromatography eluting with heptane/EtOAc=1/0 to 100/1 to provide the title compound Intermediate rac-F1-2 (4.4 kg). ¹H NMR (400 MHz, Chloroform-d) δ [ppm] 0.84-0.94 (m, 3H), 1.12 (m, J=6.60 Hz, 4H), 1.19-1.33 (m, 28H), 1.35 (s, 9H), 1.66 (quin, J=7.40 Hz, 2H), 1.85 (t, J=8.44 Hz, 4H), 2.37 (t, J=7.52 Hz, 2H), 5.14 (s, 2H), 5.16 (s, 2H), 7.30-7.42 (m, 10H).

### Step 3: 13-(Benzyloxy)-2-((benzyloxy)carbonyl)-13-oxo-2-undecyltridecanoic acid (Intermediate rac-F1)

To a solution of 1,11-dibenzyl 11-(tert-butyl) docosane-1,11,11-tricarboxylate (Intermediate rac-F1-2, 6.0 kg, 8.8 mol) in heptane (21 L) was added TFA (10.0 kg, 88.4 mol) dropwise at 20±5° C. After stirring at 20±5° C. for 8 h, most of the TFA was removed under reduced pressure. The residue was re-dissolved in heptane (42 L) and washed with brine (3×42 L). After separation of the phases, the organic phase was concentrated to provide the crude product as a yellow oil. The crude product was further purified by column chromatography eluting with heptane to heptane/EtOAc=10/1 to provide title compound Intermediate rac-F1 (4.6 kg). ¹H NMR (400 MHz, Chloroform-d) δ [ppm] 0.87-0.94 (m, 3H), 0.94-1.05 (m, 2H), 1.19 (br s, 14H), 1.23-1.37 (m, 16H), 1.65 (quin, J=7.40 Hz, 2H), 1.78-1.91 (m, 2H), 1.93-2.05 (m, 2H), 2.37 (t, J=7.52 Hz, 2H), 5.14 (s, 2H), 5.27 (s, 2H), 7.31-7.44 (m, 10H). LCMS Method W11: Rt=16.44 min/18.0 min, MS m/z [M+Na]+645.4.

### Example 19d: (R)-13-(Benzyloxy)-2-((benzyloxy)carbonyl)-13-oxo-2-undecyltridecanoic acid and (S)-13-(benzyloxy)-2-((benzyloxy)carbonyl)-13-oxo-2-undecyltridecanoic acid (Intermediate ent-F1-Peak1 and Intermediate ent-F1-Peak2)

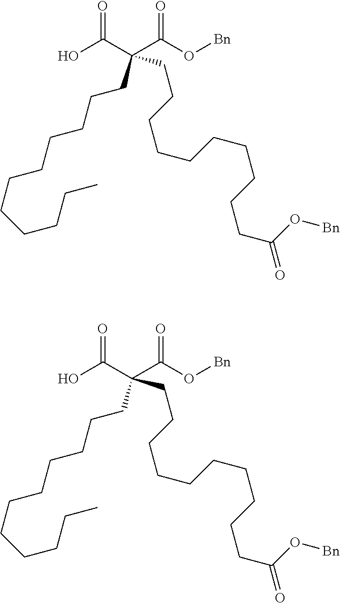 [SMILES: CCCCCCCCCCC[C@@](CCCCCCCCCCC(=O)OCc1ccccc1)(C(=O)O)C(=O)OCc1ccccc1 ; CCCCCCCCCCC[C@](CCCCCCCCCCC(=O)OCc1ccccc1)(C(=O)O)C(=O)OCc1ccccc1]

For chiral separation, 13-(benzyloxy)-2-((benzyloxy)carbonyl)-13-oxo-2-undecyltridecanoic acid (Intermediate rac-F1, 510 g) was dissolved in EtOH (25.5 L) and injected in 15 mL portions on the following Instrument: Thar 350 preparative SFC (SFC-18); Column: ChiralPak AD, 300×50 mm I.D., 10 μm; Mobile phase: A for CO₂ and B for EtOH; Gradient: B 40%; Flow rate: 200 mL/min; Back pressure: 100 bar; Column temperature: 38° C.; Wavelength: 210 nm; Cycle time: ˜3.7 min. Product fractions were concentrated under reduced pressure at 40° C. providing Enantiomer A (Intermediate ent-F1-Peak1): Eluting from the column at 4.06 min, 99.3% ee (Method C5), 222.88 g; LCMS Method C2: Rt=3.77 min/5.0 min; MS m/z [M+H]⁺=623.5; and Enantiomer B (Intermediate ent-F1-Peak2): Eluting from the column at 4.39 min, ee 98.8% (Method C5), 215.88 g; LCMS Method C2: Rt=3.75 min/5.0 min; MS m/z [M+H]⁺=623.5.

### Example 20d: 2-(((2,5-Dioxopyrrolidin-1-yl)oxy)carbonyl)-2-undecyltridecanedioic acid (Intermediate rac-F1-3)

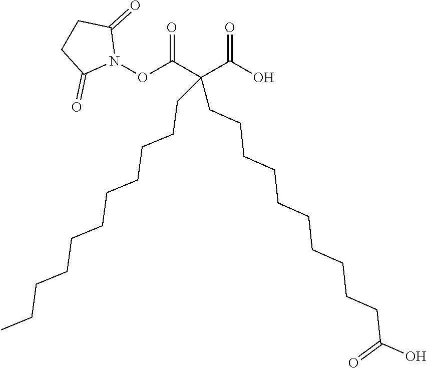 [SMILES: CCCCCCCCCCCC(CCCCCCCCCCC(=O)O)(C(=O)O)C(=O)ON1C(=O)CCC1=O]

### Step 1: 1,11-Dibenzyl 11-(2,5-dioxocyclopentyl) docosane-1,11,11-tricarboxylate (Intermediate rac-F1-3-1)

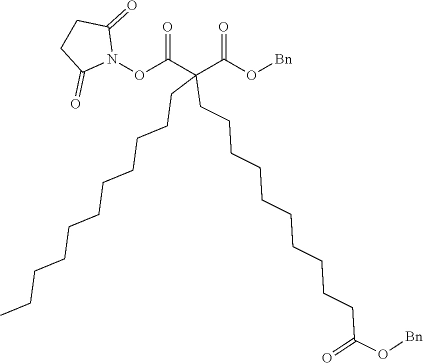 [SMILES: CCCCCCCCCCCC(CCCCCCCCCCC(=O)OCc1ccccc1)(C(=O)OCc1ccccc1)C(=O)ON1C(=O)CCC1=O]

To a 1000 mL 3-neck round bottom flask (fitted with a mechanical stirrer and N₂ inlet) was added 13-(benzyloxy)-2-((benzyloxy)carbonyl)-13-oxo-2-undecyltridecanoic acid (Intermediate rac-F1, 37.7 g, 60.5 mmol), DCM (360 mL), and THF (40 mL). To the resulting homogeneous solution was added N-hydroxysuccinimide (7.31 g, 63.6 mmol) and DCC (14.99 g, 72.6 mmol). Five min after addition, the batch had become a white suspension. The batch was stirred for 6 h at room temperature and filtered over a pad of Celite® and the pad was washed thoroughly with DCM. The combined filtrate and DCM washes were concentrated under reduced pressure, and the residue was dried under high vacuum. The crude product was isolated as a white oil. The crude product was taken up in DCM (˜400 mL) and SiO₂ (75 g) was added. The suspension was concentrated under reduced pressure and the residue dried under high vacuum for 3 h. The batch was purified via column chromatography (750 g SiO₂, eluting with 2% EtOAc/heptane to 35% EtOAc/heptane). The product containing fractions were combined, concentrated under reduced pressure and dried overnight under high vacuum to provide the title compound Intermediate rac-F1-3-1 as a colorless oil. ¹H NMR (400 MHz, Chloroform-d) δ [ppm] 0.86-0.93 (m, 3H), 1.12-1.21 (m, 2H), 1.21-1.37 (m, 30H), 1.66 (quin, J=7.40 Hz, 2H), 1.89-2.07 (m, 4H), 2.37 (t, J=7.58 Hz, 2H), 2.84 (br s, 4H), 5.13 (s, 2H), 5.25 (s, 2H), 7.30-7.47 (m, 10H).

### Step 2: 2-(((2,5-Dioxopyrrolidin-1-yl)oxy)carbonyl)-2-undecyltridecanedioic acid (Intermediate rac-F1-3)

To a 25 mL round bottom flask was added 1,11-dibenzyl 11-(2,5-dioxopyrrolidin-1-yl) docosane-1,11,11-tricarboxylate (Intermediate rac-F1-3-1, 100 mg, 0.139 mmol) and THF (2 mL). To the resulting colorless homogeneous solution was added Pd/C (7.39 mg, 6.94 μmol, 10 wt. %). The mixture was purged with H₂ and then exposed to H₂ pressure (balloon) and stirred for 30 min. The suspension was filtered over a pad of Celite® and the pad was washed thoroughly with THF. The combined filtrates were concentrated under reduced pressure, and the resulting residue was dried overnight under high vacuum to provide the crude title compound Intermediate rac-F1-3 (69 mg) as a colorless oil which was used without further purification. LCMS Method T11: Rt=1.24 min; MS m/z [M+H]⁺=540.3.

### Example 21d: 17-(Benzyloxy)-2-(15-(benzyloxy)-15-oxopentadecyl)-2-((benzyloxy)carbonyl)-17-oxoheptadecanoic acid (Intermediate F2)

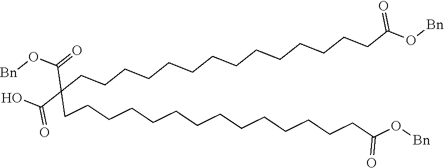 [SMILES: O=C(CCCCCCCCCCCCCCC(CCCCCCCCCCCCCCC(=O)OCc1ccccc1)(C(=O)O)C(=O)OCc1ccccc1)OCc1ccccc1]

### Step 1: Methyl 15-hydroxypentadecanoate (Intermediate F2-1)

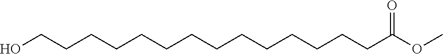 [SMILES: COC(=O)CCCCCCCCCCCCCCO]

To a solution of oxacyclohexadecan-2-one (137 g, 569 mmol) in MeOH (1 L) was added NaOMe (5 M, 42.1 mL) at 18° C. The solution was stirred at 18° C. for 21 h. TLC indicated a complete consumption of the starting material (PE/Et₂O=5/1). The RM was quenched with aq. HCl (1M, 400 mL). MeOH was removed under reduced pressure, which left behind a suspension of solids. The solids were filtered providing the crude title compound Intermediate F2-1 (134 g) which was used into the next step without further purification. ¹H NMR (400 MHz, Chloroform-d) δ [ppm] 3.67 (s, 3H), 3.64 (t, J=6.8 Hz, 2H), 2.31 (t, J=7.6 Hz, 2H), 1.52-1.67 (m, 4H), 1.21-1.39 (m, 20H).

### Step 2: Methyl 15-bromopentadecanoate (Intermediate F2-2)

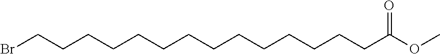 [SMILES: COC(=O)CCCCCCCCCCCCCCBr]

Methyl 15-hydroxypentadecanoate (Intermediate F2-1, 134 g, 491 mmol) was added to carbon tetrabromide (309 g, 934 mmol) and dissolved in DCM (1.5 L). The mixture was cooled to 0° C. and PPh₃ (245 g, 934 mmol) was added in portions to the mixture. The reaction was allowed to warm to 18° C. and stirred for 16 h. TLC indicated a complete consumption of the starting material (PE/Et₂O=10/1). Petroleum ether was added to the RM, which caused the precipitation of PPh₃O. The suspension was filtered through Celite® and then concentrated under reduced pressure. The resulting residue was purified by column chromatography (SiO₂, eluting with PE/Et₂O=0/1 to 20/1) providing the title compound Intermediate F2-2 (105 g) as a white solid. ¹H NMR (400 MHz, Chloroform-d) δ [ppm] 3.67 (s, 3H), 3.41 (t, J=7.0 Hz, 2H), 2.31 (t, J=7.6 Hz, 2H), 1.86 (m, 2H), 1.62 (m, 2H), 1.38-1.51 (m, 2H), 1.19-1.37 (m, 18H).

### Step 3: 15-Bromopentadecanoic acid (Intermediate F2-3)

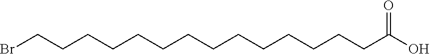 [SMILES: O=C(O)CCCCCCCCCCCCCCBr]

To a solution of methyl 15-bromopentadecanoate (Intermediate F2-2, 105 g, 315 mmol) in THF (1.5 L) was added a solution of lithium hydroxide monohydrate (66.1 g, 1.58 mol) in water (1.5 L). The resulting mixture was stirred at 18° C. for 16 h. TLC indicated a complete consumption of the starting material. The RM was diluted with aq. HCl (1M, 200 mL) and was concentrated under reduced pressure to remove the THF. The residue was diluted with water (200 mL) and extracted with DCM (3×200 mL). The combined organic layers were dried over Na₂SO₄, filtered and concentrated under reduced pressure affording the crude title compound Intermediate F2-3 (80.5 g) as a white solid which was used in the next step without further purification. ¹H NMR (400 MHz, Chloroform-d) δ [ppm] 3.41 (t, J=7.0 Hz, 2H), 2.36 (t, J=7.6 Hz, 2H), 1.86 (m, 2H), 1.59-1.69 (m, 2H), 1.39-1.48 (m, 2H), 1.27 (m, 18H).

### Step 4: Benzyl 15-bromopentadecanoate (Intermediate F2-4)

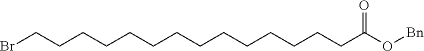 [SMILES: O=C(CCCCCCCCCCCCCCBr)OCc1ccccc1]

To a mixture of crude 15-bromopentadecanoic acid (Intermediate F2-3, 80.5 g) and BnOH (40.6 g, 375 mmol) in DCM (1.5 L) was added EDCl (72.0 g, 375 mmol) and DMAP (3.06 g, 25.0 mmol) in one portion at 18° C. The mixture was stirred at 18° C. for 16 h. TLC indicated a complete consumption of the starting material (PE/Et₂O=5/1). The RM was poured into water (1 L) and extracted with DCM (3×300 mL). The combined organic layers were washed with brine (2×500 mL), dried over Na₂SO₄, filtered and concentrated under reduced pressure. The resulting residue was purified by column chromatography (SiO₂, eluting with PE/Et₂O=1/0 to 50/1) providing the title compound Intermediate F2-4 (74.7 g) as a white solid. ¹H NMR (400 MHz, Chloroform-d) δ [ppm] 7.28-7.42 (m, 5H), 5.12 (s, 2H), 3.42 (t, J=6.8 Hz, 2H), 2.36 (t, J=7.6 Hz, 2H), 1.86 (m, 2H), 1.65 (m, 2H), 1.38-1.49 (m, 2H), 1.20-1.35 (m, 18H).

### Step 5: 1,15,29-Tribenzyl 15-(tert-butyl) nonacosane-1,15,15,29-tetracarboxylate (Intermediate F2-5)

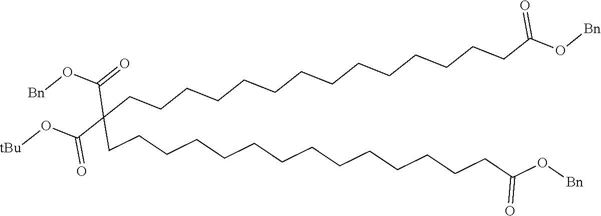 [SMILES: CC(C)(C)OC(=O)C(CCCCCCCCCCCCCCC(=O)OCc1ccccc1)(CCCCCCCCCCCCCCC(=O)OCc1ccccc1)C(=O)OCc1ccccc1]

Two parallel reactions of benzyl 15-bromopentadecanoate (Intermediate F2-4, 100 g scale and 38.7 g scale) with benzyl tert-butyl malonate were performed. The 100 g scale reaction is described up to the combination of the two parallel reactions for purification.

To a mixture of NaH (16.8 g, 422 mmol, 60 wt. %) and NaI (3.31 g, 22.1 mmol) in DMF (1.00 L) was added benzyl tert-butyl malonate (27.6 g, 110 mmol) and benzyl 15-bromopentadecanoate (Intermediate F2-4, 100 g, 243.0 mmol) in DMF (500 mL) at 0° C. The RM was stirred at 0° C. for 1 h, allowed to warm up to 18° C. and stirred for additional 23 h. The RM was poured into ice water (4.5 L) and extracted with Et₂O (3×1 L). The combined organic layers were washed with brine (2×1 L), dried over Na₂SO₄, filtered and concentrated under reduced pressure providing a crude residue (100 g). The crude residues from the two parallel reactions (100 g and 39 g, respectively) were combined and purified by column chromatography (SiO₂, eluting with PE/Et₂O=1/0 to 50/1) to provide the title compound Intermediate F2-5 (92.4 g). ¹H NMR (400 MHz, Chloroform-d) δ [ppm] 7.29-7.43 (m, 15H), 5.15 (s, 2H), 5.12 (s, 4H), 2.36 (t, J=7.6 Hz, 4H), 1.84 (t, J=8.4 Hz, 4H), 1.62-1.68 (m, 4H), 1.34 (s, 9H), 1.21-1.32 (m, 44H).

### Step 6: 17-(Benzyloxy)-2-(15-(benzyloxy)-15-oxopentadecyl)-2-((benzyloxy)carbonyl)-17-oxoheptadecanoic acid (Intermediate F2)

To a solution of 1,15,29-tribenzyl 15-(tert-butyl) nonacosane-1,15,15,29-tetracarboxylate (Intermediate F2-5, 92.4 g, 101 mmol) in DCM (1.0 L) was added TFA (115 g, 1.01 mol). The mixture was stirred at 18° C. for 24 h. The RM was diluted with aq. sat. NaHCO₃ solution (0.6 L) at 18° C. and extracted with DCM (3×200 mL). The combined organic layers were washed with brine (1 L), dried over Na₂SO₄, filtered and concentrated under reduced pressure providing the crude title compound Intermediate F2 (81.8 g) which was used directly in the next step without further purification. ¹H NMR (400 MHz, Chloroform-d) δ [ppm] 7.28-7.46 (m, 15H), 5.24 (s, 2H), 5.12 (s, 4H), 2.36 (t, J=7.6 Hz, 4H), 1.92-2.03 (m, 2H), 1.79-1.90 (m, 2H), 1.59-1.71 (m, 4H), 1.13-1.35 (m, 44H). LCMS Method C3: Rt=1.24 min, MS m/z [M+H]⁺=855.6

### Example 22d: 1-Benzyl 3-(2,5-dioxopyrrolidin-1-yl) 2,2-bis(14-(bis(benzyloxy)phosphoryl)tetradecyl)malonate (Intermediate F3)

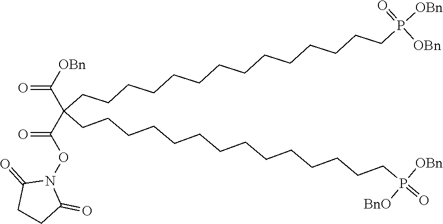 [SMILES: O=C1CCC(=O)N1OC(=O)C(CCCCCCCCCCCCCCP(=O)(OCc1ccccc1)OCc1ccccc1)(CCCCCCCCCCCCCCP(=O)(OCc1ccccc1)OCc1ccccc1)C(=O)OCc1ccccc1]

### Step 1: Dibenzyl (14-bromotetradecyl)phosphonate (Intermediate F3-1)

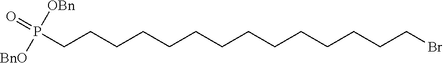 [SMILES: O=P(CCCCCCCCCCCCCCBr)(OCc1ccccc1)OCc1ccccc1]

A solution of dibenzyl phosphonate (491 mg, 1.411 mmol) in anhydrous DMF (5.5 mL) was treated carefully with sodium hydride (60% in mineral oil, 90 mg). The RM was allowed to stir for 1 h whereupon the suspended solids nearly completely dissolved. To this slightly turbid solution was added 1,14-dibromotetradecane (1.00 g, 2.81 mmol) in a single portion and the RM left to stir at RT for 16 h. The RM was then poured into water and extracted with EtOAc (3×). The combined organic layers were combined, washed with brine and dried over Na₂SO₄. Volatiles were removed under reduced pressure to yield a clear oil which was purified by normal phase column chromatography (SiO₂, eluting with 0-50% EtOAc in heptane). Fractions containing the desired product were combined and concentrated under reduced pressure to yield the title compound Intermediate F3-1 (600 mg). ¹H NMR (400 MHz, Chloroform-d) δ [ppm] 7.41-7.30 (m, 10H), 5.11-4.90 (m, 4H), 3.41 (t, J=6.9 Hz, 2H), 1.81-1.63 (m, 2H), 1.57 (s, 4H), 1.41 (t, J=7.6 Hz, 2H), 1.25 (s, 18H).

### Step 2: 1-Benzyl 3-tert-butyl 2,2-bis(14-(bis(benzyloxy)phosphoryl)tetradecyl)malonate (Intermediate F3-2)

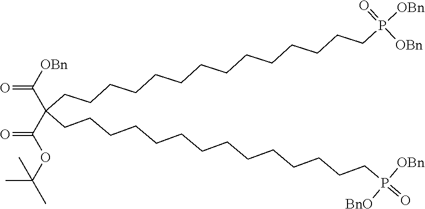 [SMILES: CC(C)(C)OC(=O)C(CCCCCCCCCCCCCCP(=O)(OCc1ccccc1)OCc1ccccc1)(CCCCCCCCCCCCCCP(=O)(OCc1ccccc1)OCc1ccccc1)C(=O)OCc1ccccc1]

Dibenzyl (14-bromotetradecyl)phosphonate (Intermediate F3-1, 337 mg, 0.627 mmol), benzyl tert-butyl malonate (71 mg, 0.284 mmol) and cesium carbonate (370 mg, 1.13 mmol) were combined in anhydrous DMF (1.18 mL) and heated to 80° C. overnight under N₂ atmosphere. The mixture was partitioned between water and EtOAc and the aqueous layer extracted with EtOAc (3×10 mL). The combined organic layers were washed with brine and dried over Na₂SO₄, then the volatiles were removed under reduced pressure. The resulting residue was purified by normal phase column chromatography (SiO₂, eluting with 0 to 60% EtOAc in heptane) to yield the title compound Intermediate F3-2 (70 mg) as a clear viscous oil. LCMS Method T11: Rt=1.73 min; MS m/z [M+H]⁺=1163.7.

### Step 3: 2-((Benzyloxy)carbonyl)-16-(bis(benzyloxy)phosphoryl)-2-(14-(bis(benzyloxy)phosphoryl)tetradecyl)hexadecanoic acid (Intermediate F3-3)

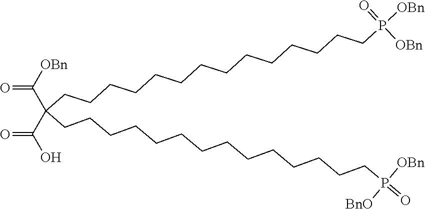 [SMILES: O=C(O)C(CCCCCCCCCCCCCCP(=O)(OCc1ccccc1)OCc1ccccc1)(CCCCCCCCCCCCCCP(=O)(OCc1ccccc1)OCc1ccccc1)C(=O)OCc1ccccc1]

A solution of 1-benzyl 3-tert-butyl 2,2-bis(14-(bis(benzyloxy)phosphoryl)tetradecyl)malonate (Intermediate F3-2, 85 mg, 0.07 mmol) in anhydrous DCM (0.7 mL) was treated with TFA (81 μL, 1.04 mmol) and the RM allowed to stir at RT for 16 h. The volatiles were removed under a gentle stream of N₂ and the resulting residue was purified by normal phase column chromatography (SiO₂, eluting with 0 to 100% EtOAc in heptane) to yield the title compound Intermediate F3-3 (65 mg). LCMS Method T11: Rt=3.92 min; MS m/z [M−H]⁻=1163.0.

### Step 4: 1-Benzyl 3-(2,5-dioxopyrrolidin-1-yl) 2,2-bis(14-(bis(benzyloxy)phosphoryl)tetradecyl)malonate (Intermediate F3)

To a solution of 2-((benzyloxy)carbonyl)-16-(bis(benzyloxy)phosphoryl)-2-(14-(bis(benzyloxy)phosphoryl)tetradecyl)hexadecanoic acid (Intermediate F3-3, 5.33 g, 4.81 mmol) and 1-hydroxypyrrolidine-2,5-dione (609 mg, 5.29 mmol) in anhydrous DCM (48 mL) was added a solution of DCC in DCM (1M, 5.29 mL). The RM was stirred at RT overnight and then concentrated under reduced pressure. The resulting residue was purified by normal phase column chromatography (SiO₂, eluting with 0 to 10% MeOH in DCM) to yield the title compound Intermediate F3 (5.58 g) as a waxy semisolid. LCMS Method T7: Rt=1.83 min; MS m/z [M+H]+=1204.7.

### Example 23d: 1-Benzyl 5-(perfluorophenyl) N-(11-(benzyloxy)-11-oxoundecanoyl)-N-undecyl-L-glutamate (Intermediate F4-S)

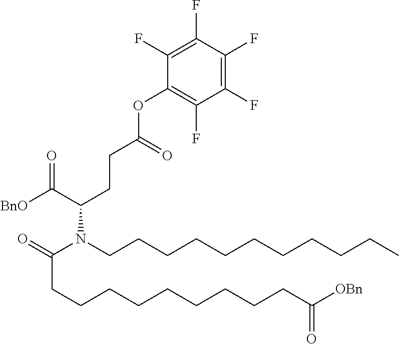 [SMILES: CCCCCCCCCCCN(C(=O)CCCCCCCCCC(=O)OCc1ccccc1)[C@@H](CCC(=O)Oc1c(F)c(F)c(F)c(F)c1F)C(=O)OCc1ccccc1]

### Step 1: 1-Benzyl 5-(tert-butyl) (((9H-fluoren-9-yl)methoxy)carbonyl)-L-glutamate (Intermediate F4-S-1)

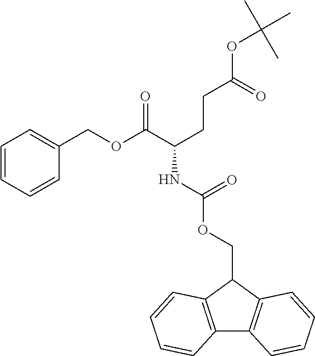 [SMILES: CC(C)(C)OC(=O)CC[C@H](NC(=O)OCC1c2ccccc2-c2ccccc21)C(=O)OCc1ccccc1]

To a suspension of (S)-2-((((9H-fluoren-9-yl)methoxy)carbonyl)amino)-5-(tert-butoxy)-5-oxopentanoic acid (500 g, 1175.11 mmol) and sodium bicarbonate (493.5 g, 5875.57 mmol) in DMF (5 L) was added benzyl bromide (502.5 g, 2938.4 mmol) at once. The heterogeneous RM was stirred at 50° C. for 16 h and then quenched with ice water (10 L). Stirring was continued for 3 h and the solids removed by filtration. The filter cake was washed sequentially with water (5 L) and petroleum ether (3 L) and then dried in vacuo to give the title compound Intermediate F4-S-1 as an off-white solid (530 g). LCMS Method G6: Rt=2.88 min; MS m/z [M+H]⁺=516.38.

### Step 2: 1-Benzyl 5-(tert-butyl) L-glutamate (Intermediate F4-S-2)

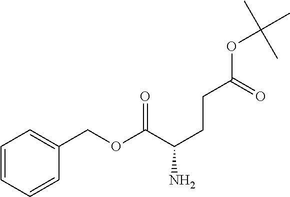 [SMILES: CC(C)(C)OC(=O)CC[C@H](N)C(=O)OCc1ccccc1]

To a solution of 1-benzyl 5-(tert-butyl) (((9H-fluoren-9-yl)methoxy)carbonyl)-L-glutamate (Intermediate F4-S-1, 1 kg, 1941.7 mmol) in DCM (5 L) was added 1,8-diazabicyclo[5.4.0]undec-7-ene (147.7 g, 970.87 mmol) and the RM stirred at RT for 3 h. The crude mixture was then transferred to a separatory funnel and diluted with DCM (5 L). The organic layer was washed with water (5 L), sat. aq. brine (5 L), dried over Na₂SO₄, filtered and then concentrated under reduced pressure. Purification by flash column chromatography over SiO₂ (100-200 mesh), eluting with 25 to 30% EtOAc in PE gave the title compound Intermediate F4-S-2 as a yellow oil (480 g). LCMS Method G5: Rt=2.01 min; MS m/z [M+H]⁺=294.12.

### Step 3: 1-Benzyl 5-(tert-butyl) undecyl-L-glutamate (Intermediate F4-S-3)

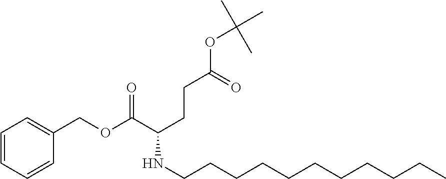 [SMILES: CCCCCCCCCCCN[C@@H](CCC(=O)OC(C)(C)C)C(=O)OCc1ccccc1]

To a stirring solution of 1-benzyl 5-(tert-butyl) L-glutamate (Intermediate F4-S-2, 480.0 g, 1636.2 mmol) in DMF (4.8 L) was added potassium carbonate (451 g, 2454.3 mmol), sodium iodide (245.2 g, 1636.2 mmol) and 1-bromoundecane (577.2 g, 1178.5 mmol). The resulting mixture was stirred at 50° C. for 16 h and then cooled to RT. The solids were removed by filtration and the filter cake washed with EtOAc (5 L). The filtrate was transferred to a separatory funnel and washed with water (3×5 L), brine (5 L), dried over Na₂SO₄, filtered and then concentrated under reduced pressure. Purification by flash column chromatography over SiO₂ (100-200 mesh), eluting with 10% EtOAc in PE gave the title compound Intermediate F4-S-3 as a yellow oil (460 g).

### Step 4: 11-(Benzyloxy)-11-oxoundecanoic acid (Intermediate F4-S-4)

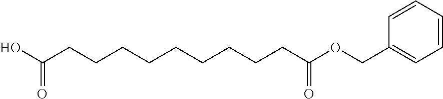 [SMILES: O=C(O)CCCCCCCCCC(=O)OCc1ccccc1]

To a stirring solution of undecanedioic acid (900 g, 4.16 mol) in toluene (3550 mL) was added benzyl alcohol (335 mL, 4.07 mol) and p-toluenesulfonic acid monohydrate (62 g, 0.416 mol). The RM was refluxed for 4 h and then cooled to RT. The crude mixture was then transferred to a separatory funnel and diluted with EtOAc (2150 mL). The organic layer was washed with water (1075 mL), brine (1075 mL), dried over Na₂SO₄, filtered and then concentrated under reduced pressure. Purification by flash column chromatography over SiO₂ (100-200 mesh), eluting with 6 to 8% EtOAc in PE gave the title compound Intermediate F4-S-4 as a yellow liquid (550 g).

### Step 5a: Benzyl 11-chloro-11-oxoundecanoate (Intermediate F4-S-5a)

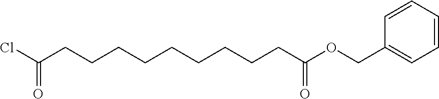 [SMILES: O=C(Cl)CCCCCCCCCC(=O)OCc1ccccc1]

A stirring solution of 11-(benzyloxy)-11-oxoundecanoic acid (Intermediate F4-S-4, 480.0 g, 1568.6 mmol) in DCM (5 L) was cooled to 0° C. and thionyl chloride (560 g, 4705.8 mmol) added gradually, followed by the addition of DMF (5 mL). The resulting mixture was stirred at 45° C. for 3 h and then concentrated under reduced pressure. The crude title compound Intermediate F4-S-5a (495 g) was used directly in the next step without purification.

### Step 5b: 1-Benzyl 5-(tert-butyl) N-(11-(benzyloxy)-11-oxoundecanoyl)-N-undecyl-L-glutamate (Intermediate F4-S-5b)

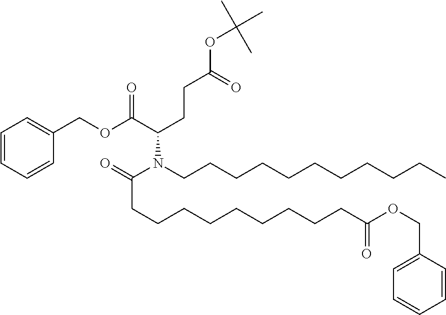 [SMILES: CCCCCCCCCCCN(C(=O)CCCCCCCCCC(=O)OCc1ccccc1)[C@@H](CCC(=O)OC(C)(C)C)C(=O)OCc1ccccc1]

A stirring solution of benzyl 11-chloro-11-oxoundecanoate (Intermediate F4-S-5, 460 g, 1027.7 mmol) in DCM (4.6 L) was cooled to 0° C. DIPEA (662 g, 5138.5 mmol) was added followed by a solution of 1-benzyl 5-(tert-butyl) undecyl-L-glutamate (Intermediate F4-S-3, 400 g, 1233.2 mmol) in DCM (4.6 L) over a period of 2 h. The RM was then allowed to warm to RT and stirring continued for a further 16 h. The RM was quenched with MeOH and then concentrated under reduced pressure. Purification by flash column chromatography over SiO₂ (100-200 mesh), eluting with 3 to 5% EtOAc in PE gave the title compound Intermediate F4-S-5b (410 g) as a brown liquid. LCMS Method G2: Rt=4.52 min; MS m/z [M+H]⁺=736.27.

### Step 6: (S)-5-(Benzyloxy)-4-(11-(benzyloxy)-11-oxo-N-undecylundecanamido)-5-oxopentanoic acid (Intermediate F4-S-6)

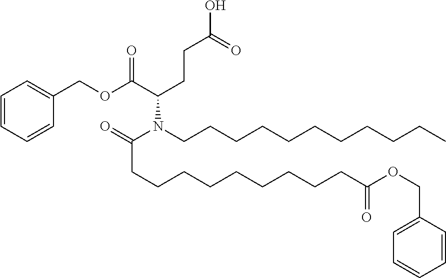 [SMILES: CCCCCCCCCCCN(C(=O)CCCCCCCCCC(=O)OCc1ccccc1)[C@@H](CCC(=O)O)C(=O)OCc1ccccc1]

To a stirring solution of 1-benzyl 5-(tert-butyl) N-(11-(benzyloxy)-11-oxoundecanoyl)-N-undecyl-L-glutamate (Intermediate F4-S-5b, 400 g, 543.47 mmol) and triisopropylsilane (129 g, 815.2 mmol) in DCM (4 L) was added TFA (929.3 g, 8152.2 mmol) and the resulting mixture stirred for 8 h at RT. The crude mixture was then transferred to a separatory funnel and diluted with DCM (5 L). The organic layer was washed with water (5 L), brine (5 L), dried over Na₂SO₄, filtered and then concentrated under reduced pressure. Purification by flash column chromatography over SiO₂ (100-200 mesh), eluting with 30 to 40% EtOAc in PE gave the title compound Intermediate F4-S-6 (320 g) as a yellow oil. LCMS Method G3: Rt=5.33 min; MS m/z [M+H]⁺=680.15.

### Step 7: 1-Benzyl 5-(perfluorophenyl) N-(11-(benzyloxy)-11-oxoundecanoyl)-N-undecyl-L-glutamate (Intermediate F4-S)

A stirring solution of (S)-5-(benzyloxy)-4-(11-(benzyloxy)-11-oxo-N-undecylundecanamido)-5-oxopentanoic acid (Intermediate F4-S-6, 320 g, 470.6 mmol) in DCM (3.2 L) was cooled to 0° C. and pentafluorophenol (103.9 g, 564.76 mmol), EDC.HCl (180.4 g, 941.2 mmol) and DMAP (11.48 g, 94.1 mmol) added sequentially. The resulting mixture was stirred at RT for 4 h and then transferred to a separatory funnel and diluted with DCM (3 L). The organic layer was washed with water (3 L), brine (3 L), dried over Na₂SO₄, filtered and then concentrated under reduced pressure. Purification by flash column chromatography over SiO₂ (Davisil 40-60 micron), eluting with 8 to 10% EtOAc in PE gave the title compound Intermediate F4-S (225 g) as a yellow liquid. LCMS Method G1: Rt=4.26 min; MS m/z [M+H]⁺=846.59, Chiral Method G8: Rt=4.45 min, ee 98.2%.

### Example 24d: 1-Benzyl 5-(perfluorophenyl) N-(11-(benzyloxy)-11-oxoundecanoyl)-N-undecyl-D-glutamate (Intermediate F4-R)

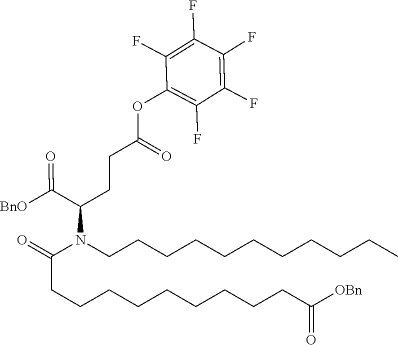 [SMILES: CCCCCCCCCCCN(C(=O)CCCCCCCCCC(=O)OCc1ccccc1)[C@H](CCC(=O)Oc1c(F)c(F)c(F)c(F)c1F)C(=O)OCc1ccccc1]

This compound was prepared, using the R-isomer, according to the procedures described for Intermediate F4-S in Example 23d herein above. LCMS Method G7: (ELSD Signal) Rt=6.19 min; m/z [M+H]⁺=846.48, Chiral Method G9: Rt=3.78 min, ee 92.6%.

### Example 25d: 2-((27-((2,5-Dioxopyrrolidin-1-yl)oxy)-27-oxo-3,6,9,12,15,18,21,24-octaoxaheptacosyl)carbamoyl)-2-undecyltridecanedioic acid (Intermediate rac-F1-Peg8)

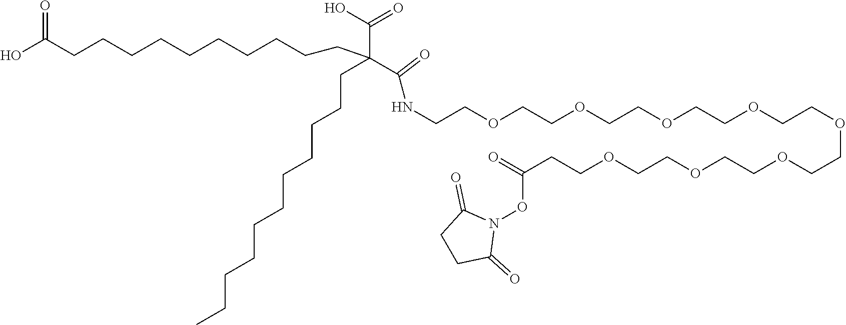 [SMILES: CCCCCCCCCCCC(CCCCCCCCCCC(=O)O)(C(=O)O)C(=O)NCCOCCOCCOCCOCCOCCOCCOCCOCCC(=O)ON1C(=O)CCC1=O]

### Step 1: 14-((Benzyloxy)carbonyl)-3,15-dioxo-1-phenyl-14-undecyl-2,19,22,25,28,31,34,37,40-nonaoxa-16-azatritetracontan-43-oic acid (Intermediate rac-F1-Peg8-1)

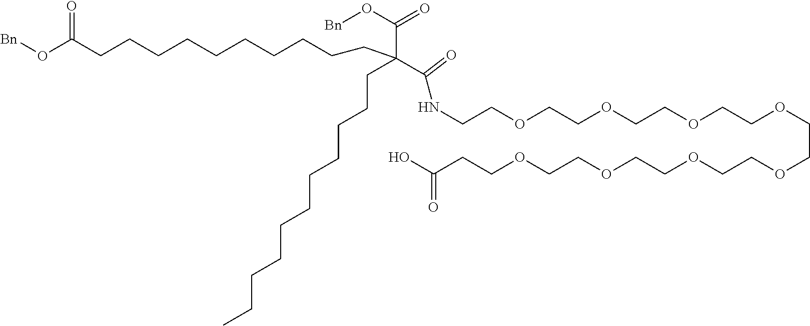 [SMILES: CCCCCCCCCCCC(CCCCCCCCCCC(=O)OCc1ccccc1)(C(=O)NCCOCCOCCOCCOCCOCCOCCOCCOCCC(=O)O)C(=O)OCc1ccccc1]

To a 250 mL round bottom flask (fitted with a magnetic stirrer and N₂ inlet) was added 1,11-dibenzyl 11-(2,5-dioxocyclopentyl) docosane-1,11,11-tricarboxylate (Intermediate rac-F1-3-1, 7.0 g, 9.72 mmol) and DCM (70 mL). To the mixture was added 1-amino-3,6,9,12,15,18,21,24-octaoxaheptacosan-27-oic acid (4.51 g, 10.21 mmol), DIPEA (4.25 mL, 24.31 mmol), and DMAP (0.119 g, 0.972 mmol). The light yellow homogeneous solution was stirred overnight at ambient temperature. The batch was concentrated under reduced pressure to give a light yellow oil. The residue was diluted with EtOAc (150 mL) and washed with brine (500 mL). After separation, the resulting aq. phase was back-extracted 2× with EtOAc (150 mL; then 100 mL). The combined organic phases were dried over Na₂SO₄, filtered over Celite®, and concentrated under reduced pressure. The crude product was purified via column chromatography (330 g SiO₂, eluting with DCM to 10% MeOH/DCM). The product fractions were combined, concentrated under reduced pressure and dried overnight under high vacuum to give the title compound Intermediate rac-F1-Peg8-1. LCMS Method T11: Rt=1.43 min; MS m/z [M+H]+ 1047.0.

### Step 2: 29,39-Dibenzyl 1-(2,5-dioxopyrrolidin-1-yl) 28-oxo-29-undecyl-3,6,9,12,15,18,21,24-octaoxa-27-azanonatriacontane-1,29,39-tricarboxylate (Intermediate rac-F1-Peg8-2)

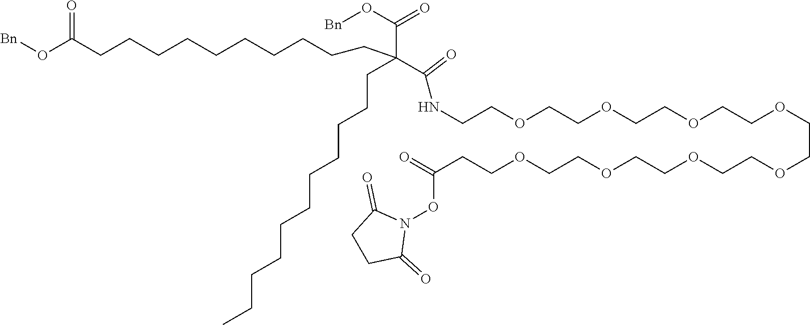 [SMILES: CCCCCCCCCCCC(CCCCCCCCCCC(=O)OCc1ccccc1)(C(=O)NCCOCCOCCOCCOCCOCCOCCOCCOCCC(=O)ON1C(=O)CCC1=O)C(=O)OCc1ccccc1]

To a solution of 14-((benzyloxy)carbonyl)-3,15-dioxo-1-phenyl-14-undecyl-2,19,22,25,28,31,34,37,40-nonaoxa-16-azatritetracontan-43-oic acid (Intermediate rac-F1-Peg8-1, 5.51 g, 5.27 mmol) in DCM (27.5 mL) and THF (27.5 mL) was added DCC (1.412 g, 6.85 mmol) and N-hydroxysuccinimide (0.697 g, 6.06 mmol). After stirring for approximately 10 min the batch became a thick white suspension. The batch was stirred for 3.75 h at ambient temperature and then concentrated under reduced pressure to a white paste. DCM (35 mL) was added to the white paste and the resulting white suspension was stirred for 10 min. The mixture was then filtered over a pad of Celite® and the pad was washed with cold DCM (one bed volume). The combined filtrates were concentrated under reduced pressure and dried overnight under high vacuum to provide the title compound Intermediate rac-F1-Peg8-2 as a colorless oil. LCMS Method T11: Rt=1.45 min; MS m/z [M+H]+ 1144.0.

### Step 3: 2-((27-((2,5-Dioxopyrrolidin-1-yl)oxy)-27-oxo-3,6,9,12,15,18,21,24-octaoxaheptacosyl)carbamoyl)-2-undecyltridecanedioic acid (Intermediate rac-F1-Peg8)

To a 250 mL round bottom flask (fitted with a magnetic stirrer) was added 29,39-dibenzyl 1-(2,5-dioxopyrrolidin-1-yl) 28-oxo-29-undecyl-3,6,9,12,15,18,21,24-octaoxa-27-azanonatriacontane-1,29,39-tricarboxylate (Intermediate rac-F1-Peg8-2, 6.0 g, 5.25 mmol) and THF (70 mL). To this solution was added Pd/C (10 wt. %, 0.603 g, 0.567 mmol), and the reaction vessel was purged with N₂ and H₂. The batch was then exposed to H₂ (balloon pressure) for 3 h. The reaction vessel was purged with N₂ and the suspension was filtered over a pad of Celite®. The pad was washed thoroughly with THF. The combined filtrates were concentrated under reduced pressure, and dried overnight under high vacuum to provide the title compound Intermediate rac-F1-Peg8 as a colorless oil. LCMS Method T11: Rt=0.91 min; MS m/z [M+H]⁺963.8.

### Example 26d: 2-((39-((2,5-Dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate rac-F1-Peg12)

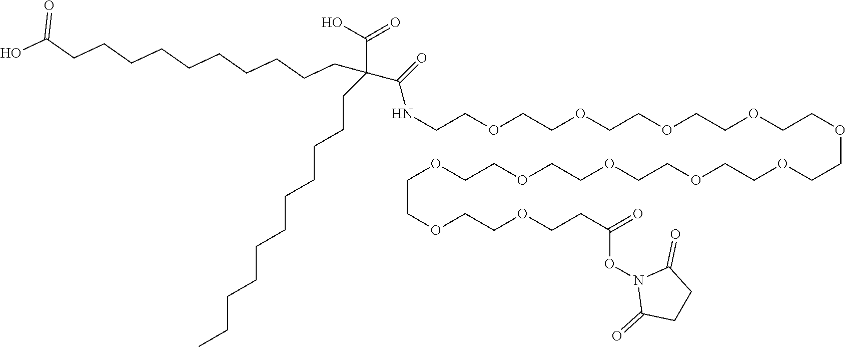 [SMILES: CCCCCCCCCCCC(CCCCCCCCCCC(=O)O)(C(=O)O)C(=O)NCCOCCOCCOCCOCCOCCOCCOCCOCCOCCOCCOCCOCCC(=O)ON1C(=O)CCC1=O]

### Step 1: Dibenzyl 2-(chlorocarbonyl)-2-undecyltridecanedioate (Intermediate rac-F1-Peg12-1)

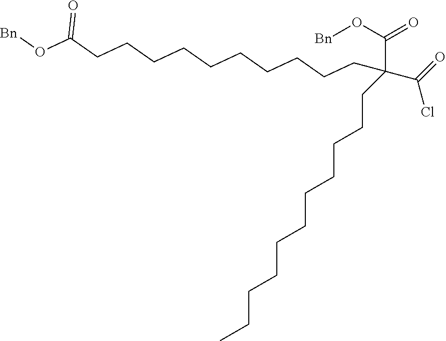 [SMILES: CCCCCCCCCCCC(CCCCCCCCCCC(=O)OCc1ccccc1)(C(=O)Cl)C(=O)OCc1ccccc1]

To solution of 13-(benzyloxy)-2-((benzyloxy)carbonyl)-13-oxo-2-undecyltridecanoic acid (Intermediate rac-F1, 5 g, 8.03 mmol) in DCM (25 mL) under N₂ atmosphere at RT was added DMF (0.012 mL, 0.161 mmol) followed by dropwise addition of oxalyl chloride (0.913 mL, 10.4 mmol) over 5 min. Upon addition of oxalyl chloride, the mixture became cloudy and gas evolution started. The mixture was stirred at RT for 2.25 h. The mixture was then concentrated under reduced pressure to give a colorless oil. To this oil was added 6 mL of heptane and concentrated under reduced pressure to give a colorless oil with some solid particles (5.8 g). The oil was then diluted with DCM (50 mL) and the insoluble particles were filtered. The resulting DCM filtrate containing the title compound Intermediate rac-F1-Peg12-1 which was used in the next step without further purification.

### Step 2: 14-((Benzyloxy)carbonyl)-3,15-dioxo-1-phenyl-14-undecyl-2,19,22,25,28,31,34,37,40,43,46,49,52-tridecaoxa-16-azapentapentacontan-55-oic acid (Intermediate rac-F1-Peg12-2)

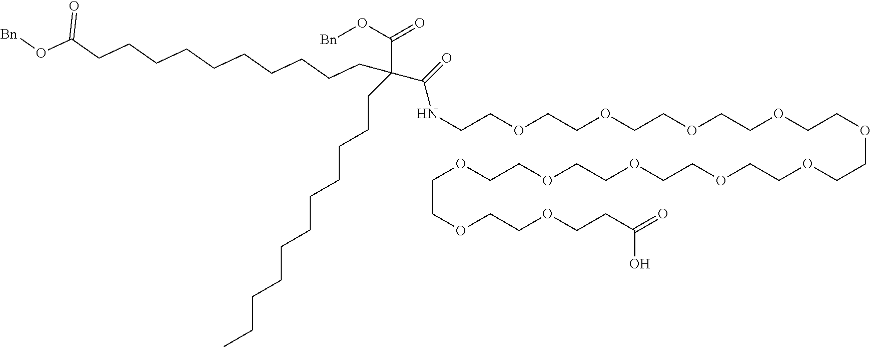 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCC(=O)O)C(=O)OCc1ccccc1]

1-amino-3,6,9,12,15,18,21,24,27,30,33,36-dodecaoxanonatriacontan-39-oic acid (5.5 g, 8.90 mmol) in DCM (58 mL) under N₂ atmosphere at RT was treated with DIPEA (3.11 mL, 17.81 mmol). This suspension slowly became a clear solution after stirring for 10 min and ultrasonicating for 5 min at RT. To the mixture was then added a solution of dibenzyl 2-(chlorocarbonyl)-2-undecyltridecanedioate (Intermediate rac-F1-Peg12-1, 5.14 g, 8.01 mmol) in DCM (50 mL) dropwise over 30 min. The RM was stirred at RT for 3 h. The RM was treated with anhydrous MgSO₄ (6.6 g) and stirred for 0.5 h. Then Dowex 50WX2 hydrogen form 50-100 mesh resin (6.6 g) was added in one portion and the mixture continued to stir at RT for an additional 0.5 h. The mixture was then filtered and the filter cake washed with DCM (50 mL). The combined filtrate and DCM wash were concentrated under reduced pressure to give an off-white oil. The oil was dried under high vacuum overnight at RT to afford a gel. This crude gel was diluted with EtOAc (250 mL) and washed with aq. HCl (0.5N, 3×100 mL). The combined aq. HCl washes were extracted with EtOAc (200 mL). The combined EtOAc layers were dried over MgSO₄, filtered, and concentrated under reduced pressure to yield 10.7 g of the title compound Intermediate rac-F1-Peg12-2 as a colorless oil. The product was used in the next step without further purification. LCMS Method T12: Rt 3.01 min; MS m/z [M+H]⁺=1223.3.

### Step 3: 41,51-Dibenzyl 1-(2,5-dioxopyrrolidin-1-yl) 40-oxo-41-undecyl-3,6,9,12,15,18,21,24,27,30,33,36-dodecaoxa-39-azahenpentacontane-1,41,51-tricarboxylate (Intermediate rac-F1-Peg12-3)

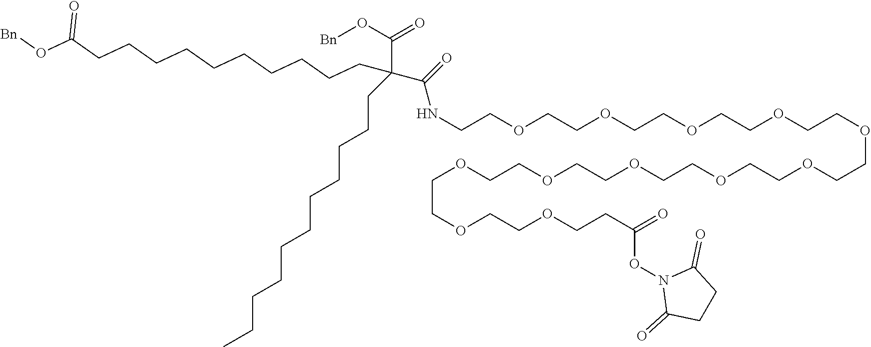 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCC(=O)ON1C(=O)CCC1=O)C(=O)OCc1ccccc1]

To a solution of 14-((benzyloxy)carbonyl)-3,15-dioxo-1-phenyl-14-undecyl-2,19,22,25,28,31,34,37,40,43,46,49,52-tridecaoxa-16-azapentapentacontan-55-oic acid (Intermediate rac-F1-Peg12-2, 9.67 g, 0.792 mmol) in DCM (65 mL) at RT was added TEA (0.329 mL, 0.160 mmol) followed by the addition of DSC (2.86 g, 11.15 mmol) in 4 portions over 2 h (0.5 eq at the beginning, 0.5 eq after 0.5 h, 0.2 eq after 0.5 h, and 0.1 eq after 1 h). The RM continued to stir at RT for ˜16 h. Dowex 50WX2 hydrogen form 50-100 mesh resin (2.57 g) was added to the RM and stirred for 0.5 h. MgSO₄ (2.57 g) was then added and the suspension stirred for an additional 0.5 h. The mixture was filtered and the filter cake was washed with DCM (50 mL). The combined filtrate and DCM wash were concentrated under reduced pressure to afford a thick, pale yellow oil. This crude product was diluted with DCM (15 mL) and purified on an Isco RediSep® 150 g silica cartridge eluting with a 0-10% MeOH/DCM gradient. Product fractions were collected and concentrated under reduced pressure to a minimal volume. To this residue was added heptane (50 mL) and the solution concentrated under reduced pressure. The residue was treated a second time with heptane (50 mL), concentrated, and dried under high vacuum to give 10.7 g of the title compound Intermediate rac-F1-Peg12-3 as a colorless oil. LCMS Method T12: Rt 3.09 min; MS m/z [M+H]⁺=1321.3.

### Step 4: 2-((39-((2,5-Dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate rac-F1-Peg12)

To a solution of 41,51-dibenzyl 1-(2,5-dioxopyrrolidin-1-yl) 40-oxo-41-undecyl-3,6,9,12,15,18,21,24,27,30,33,36-dodecaoxa-39-azahenpentacontane-1,41,51-tricarboxylate (Intermediate rac-F1-Peg12-3, 10.7 g, 8.11 mmol) in THF (150 mL) under N₂ was added anhydrous MgSO₄ (1.07 g) followed by Pd/C (10 wt. % on activated charcoal, 1.07 g, 1.00 mmol). The suspension was placed under an atmosphere of H₂ and stirred for 16 h. The RM was purged with N₂ and filtered through a pre-loaded Celite® filter that was pre-wetted with THF. The solid was rinsed 4× with THF. The combined filtrate and THF washes were concentrated under reduced pressure to afford 9.9 g of the title compound Intermediate rac-F1-Peg12 as a viscous oil. LCMS Method T11: Rt 0.93 min; MS m/z [M+H]⁺=1140.2.

### Example 27d and 27e: (R)-2-((39-((2,5-Dioxopyrrolidin-1-yl)oxy)-39-oxo-3,6,9,12,15,18,21,24,27,30,33,36-dodecaoxanonatriacontyl)carbamoyl)-2-undecyltridecanedioic acid and (S)-2-((39-((2,5-dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate ent-F1-Peak1-Peg12 and Intermediate ent-F1-Peak2-Peg12)

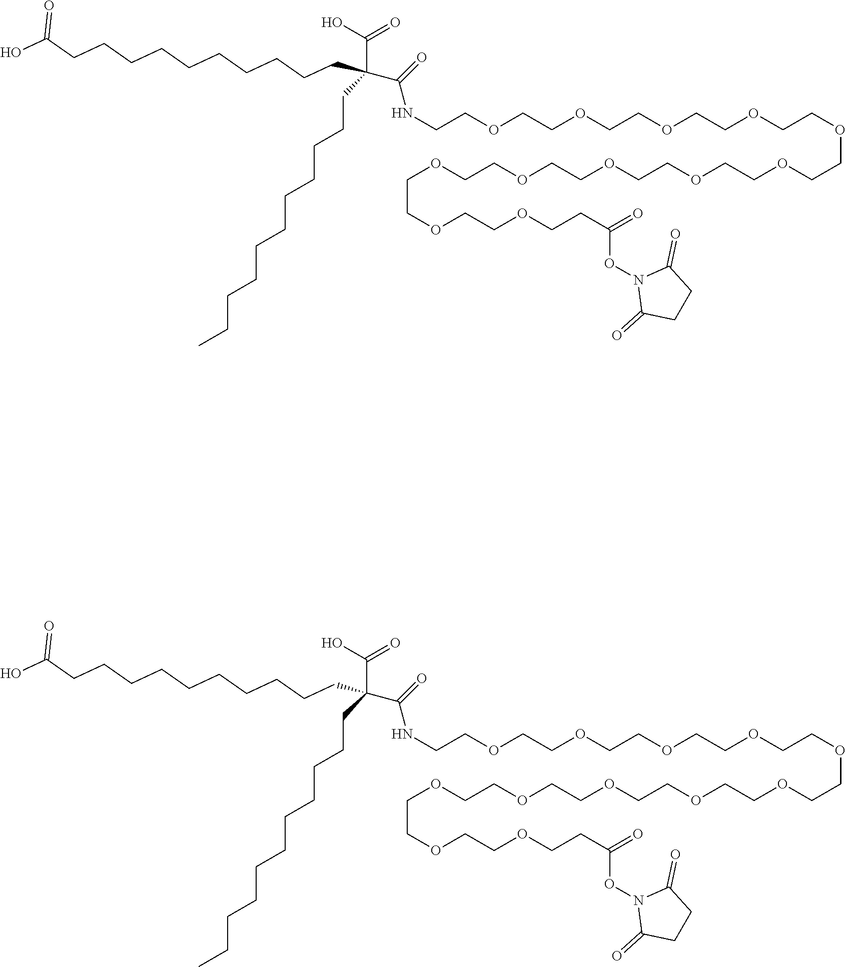 [SMILES: CCCCCCCCCCC[C@](CCCCCCCCCCC(=O)O)(C(=O)O)C(=O)NCCOCCOCCOCCOCCOCCOCCOCCOCCOCCOCCOCCOCCC(=O)ON1C(=O)CCC1=O ; CCCCCCCCCCC[C@@](CCCCCCCCCCC(=O)O)(C(=O)O)C(=O)NCCOCCOCCOCCOCCOCCOCCOCCOCCOCCOCCOCCOCCC(=O)ON1C(=O)CCC1=O]

### Step 1: Dibenzyl (R)-2-(chlorocarbonyl)-2-undecyltridecanedioate and Dibenzyl (S)-2-(chlorocarbonyl)-2-undecyltridecanedioate (Intermediate ent-F1-Peak1-Peg12-1 and Intermediate ent-F1-Peak2-Peg12-1)

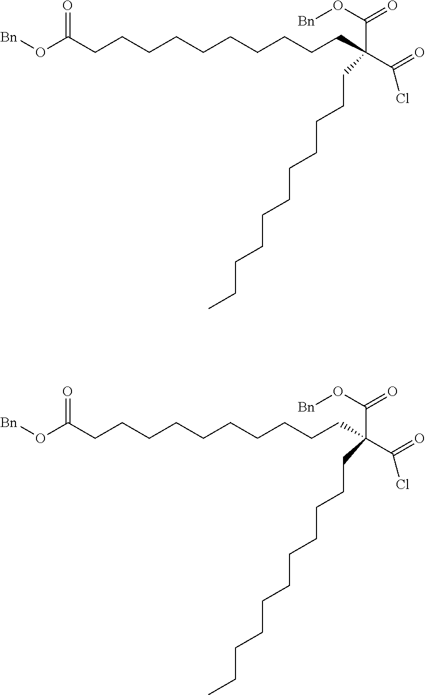 [SMILES: CCCCCCCCCCC[C@@](CCCCCCCCCCC(=O)OCc1ccccc1)(C(=O)Cl)C(=O)OCc1ccccc1 ; CCCCCCCCCCC[C@](CCCCCCCCCCC(=O)OCc1ccccc1)(C(=O)Cl)C(=O)OCc1ccccc1]

### Step 1-1: Intermediate ent-F1-Peak1-Peg12-1

To a solution of Intermediate ent-F1-Peak1 (3.04 g, 4.88 mmol) in DCM (10 mL) was added DMF (7.56 μL, 0.098 mmol) and oxalyl chloride (0.56 mL, 6.34 mmol). The RM was allowed to stir for 1 h at RT and concentrated under reduced pressure. To the resulting residue was added heptane (10 mL) and the mixture was concentrated under reduced pressure. To this resulting residue was added DCM (10 mL), the mixture was filtered and the filtrate containing the title compound Intermediate ent-F1-Peak1-Peg12-1 was used immediately in the next step without further purification.

### Step 1-2: Intermediate ent-F1-Peak2-Peg12-1

To a solution of Intermediate ent-F1-Peak2 (3.07 g, 4.93 mmol) in DCM (10 mL) was added DMF (7.63 μL, 0.099 mmol) and oxalyl chloride (0.56 mL, 6.41 mmol). The RM was allowed to stir for 2 h at RT and concentrated under reduced pressure. To the resulting residue was added heptane (10 mL) and the mixture was concentrated under reduced pressure. To this resulting residue was added DCM (10 mL) added, the mixture was filtered and the filtrate containing the title compound Intermediate ent-F1-Peak2-Peg12-1 was used immediately in the next step without further purification.

### Step 2: (R)-14-((Benzyloxy)carbonyl)-3,15-dioxo-1-phenyl-14-undecyl-2,19,22,25,28,31,34,37,40,43,46,49,52-tridecaoxa-16-azapentapentacontan-55-oic acid and (S)-14-((benzyloxy)carbonyl)-3,15-dioxo-1-phenyl-14-undecyl-2,19,22,25,28,31,34,37,40,43,46,49,52-tridecaoxa-16-azapentapentacontan-55-oic acid (Intermediate ent-F1-Peak1-Peg12-2 and Intermediate ent-F1-Peak2-Peg12-2)

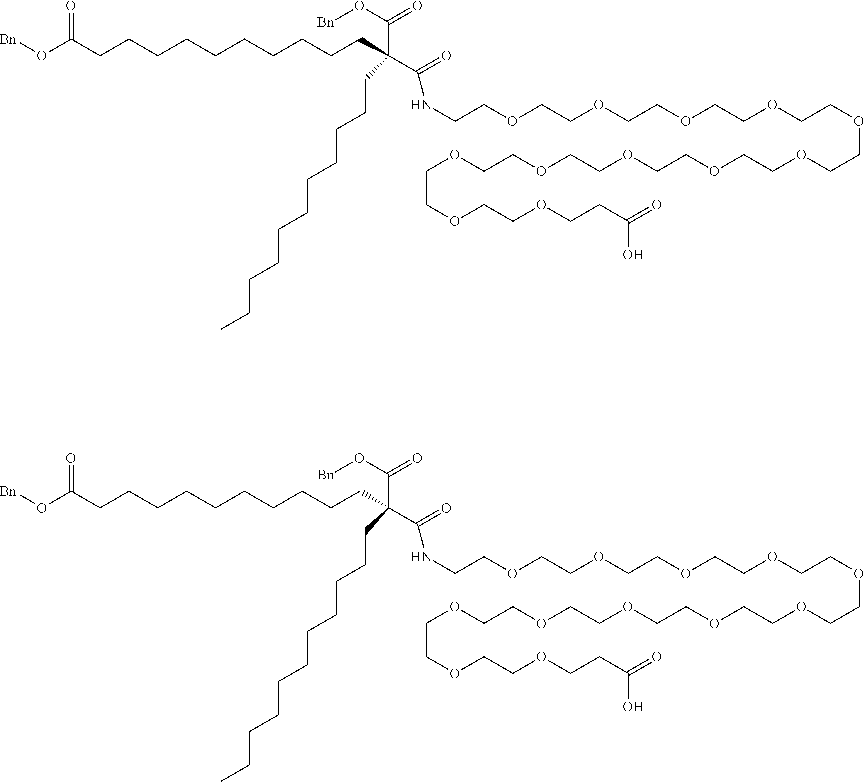 [SMILES: CCCCCCCCCCC[C@@](CCCCCCCCCCC(=O)OCc1ccccc1)(C(=O)NCCOCCOCCOCCOCCOCCOCCOCCOCCOCCOCCOCCOCCC(=O)O)C(=O)OCc1ccccc1 ; CCCCCCCCCCC[C@](CCCCCCCCCCC(=O)OCc1ccccc1)(C(=O)NCCOCCOCCOCCOCCOCCOCCOCCOCCOCCOCCOCCOCCC(=O)O)C(=O)OCc1ccccc1]

### Step 2-1: Intermediate ent-F1-Peak1-Peg12-2

To the filtrate containing Intermediate ent-F1-Peak1-Peg12-1 was added DIPEA (1.58 mL, 9.04 mmol) and 1-amino-3,6,9,12,15,18,21,24,27,30,33,36-dodecaoxanonatriacontan-39-oic acid (2.93 g, 4.75 mmol). After stirring the RM for 1 h at RT, MgSO₄ (5 g) was added and stirring was continued for 30 min. Dowex resin WX4 (100 mesh, 5 g) was added and stirring was continued for additional 30 min. The mixture was filtered, rinsed with DCM and the filtrate was concentrated under reduced pressure. The resulting crude residue containing the title compound Intermediate ent-F1-Peak1-Peg12-2 was used directly in the next step without further purification. LCMS Method T12: Rt=3.07 min; MS m/z [M+H]⁺=1223.3.

### Step 2-2: Intermediate ent-F1-Peak2-Peg12-2

To the filtrate containing Intermediate ent-F1-Peak2-Peg12-1 was added DIPEA (1.62 mL, 9.26 mmol) and 1-amino-3,6,9,12,15,18,21,24,27,30,33,36-dodecaoxanonatriacontan-39-oic acid (3.0 g, 4.75 mmol). After stirring the RM for 1 h at RT, MgSO₄ (5 g) was added and stirring was continued for 30 min. Dowex resin WX4 (100 mesh, 5 g) was added and stirring was continued for additional 30 min. The mixture was filtered, rinsed with DCM and the filtrate was concentrated under reduced pressure. The resulting crude residue containing the title compound Intermediate ent-F1-Peak2-Peg12-2 was used directly in the next step without any further purification. LCMS Method T12: Rt=3.07 min; MS m/z [M+H]⁺=1223.3.

### Step 3: 41,51-Dibenzyl 1-(2,5-dioxopyrrolidin-1-yl) (R)-40-oxo-41-undecyl-3,6,9,12,15,18,21,24,27,30,33,36-dodecaoxa-39-azahenpentacontane-1,41,51-tricarboxylate and 41,51-dibenzyl 1-(2,5-dioxopyrrolidin-1-yl) (S)-40-oxo-41-undecyl-3,6,9,12,15,18,21,24,27,30,33,36-dodecaoxa-39-azahenpentacontane-1,41,51-tricarboxylate (Intermediate ent-F1-Peak1-Peg12-3 and Intermediate ent-F1-Peak2-Peg12-3)

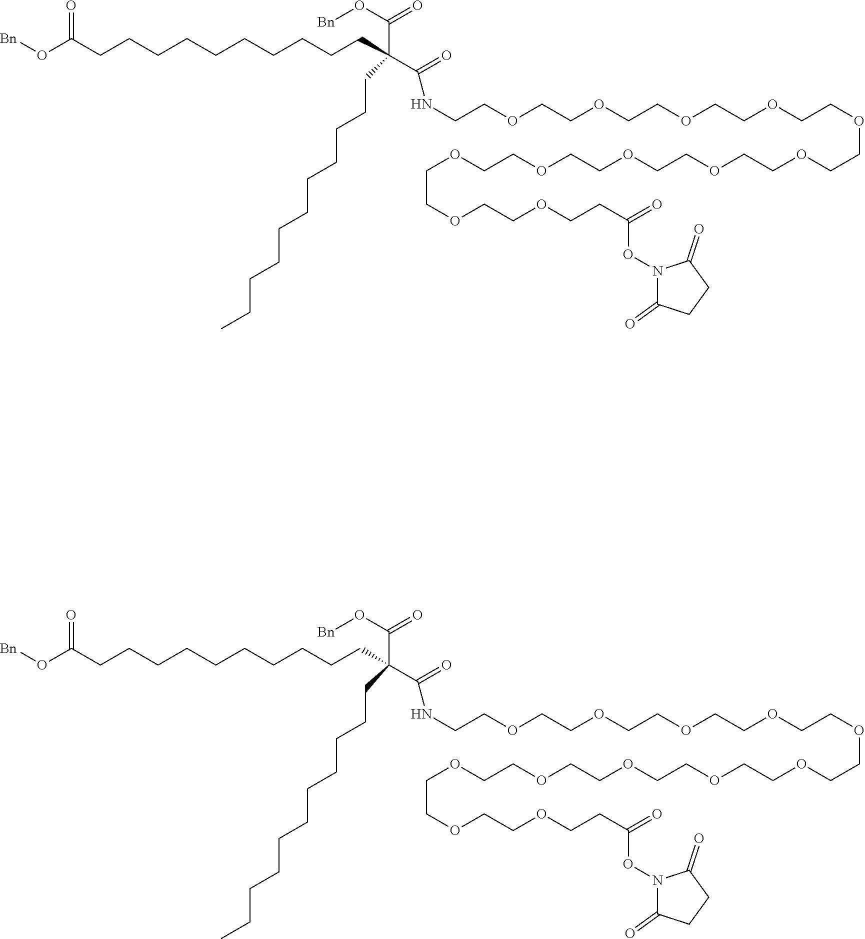 [SMILES: CCCCCCCCCCC[C@@](CCCCCCCCCCC(=O)OCc1ccccc1)(C(=O)NCCOCCOCCOCCOCCOCCOCCOCCOCCOCCOCCOCCOCCC(=O)ON1C(=O)CCC1=O)C(=O)OCc1ccccc1 ; CCCCCCCCCCC[C@](CCCCCCCCCCC(=O)OCc1ccccc1)(C(=O)NCCOCCOCCOCCOCCOCCOCCOCCOCCOCCOCCOCCOCCC(=O)ON1C(=O)CCC1=O)C(=O)OCc1ccccc1]

### Step 3-1: Intermediate ent-F1-Peak1-Peg12-3

To a solution of the crude residue containing Intermediate ent-F1-Peak1-Peg12-2 (5.53 g, 4.52 mmol) and TEA (0.126 mL, 0.905 mmol) in DCM (15 mL) was added DSC (1.51 g, 5.88 mmol) and the mixture was allowed to stir for 16 h at RT under an N₂ atmosphere. To the RM was added Dowex resin WX4 (100 mesh, 1 g) and stirring was continued for 30 min. To the mixture was added MgSO₄ (5 g) and stirring was continued for 30 min. The mixture was filtered, rinsed with DCM and the filtrate was concentrated under reduced pressure. The resulting crude residue containing the title compound Intermediate ent-F1-Peak1-Peg12-3 (6.31 g) was used directly in the next step without further purification. LCMS Method T11: Rt=1.53 min; MS m/z [M+H]⁺=1320.4.

### Step 3-2: Intermediate ent-F1-Peak2-Peg12-3

To a solution of the crude residue containing Intermediate ent-F1-Peak2-Peg12-2 (5.53 g, 4.52 mmol) and TEA (0.126 mL, 0.905 mmol) in DCM (15 mL) was added DSC (1.51 g, 5.88 mmol) and the mixture was allowed to stir for 16 h at RT under an N₂ atmosphere. To the RM was added Dowex resin WX4 (100 mesh, 1 g) and stirring was continued for 30 min. To the mixture was added MgSO₄ (5 g) and stirring was continued for 30 min. The mixture was filtered, rinsed with DCM and the filtrate was concentrated under reduced pressure. The resulting crude residue containing the title compound Intermediate ent-F1-Peak2-Peg12-3 (6.24 g) was used directly in the next step without further purification. LCMS Method T11: Rt=1.52 min; MS m/z [M+H]⁺=1320.4.

### Step 4: (R)-2-((39-((2,5-Dioxopyrrolidin-1-yl)oxy)-39-oxo-3,6,9,12,15,18,21,24,27,30,33,36-dodecaoxanonatriacontyl)carbamoyl)-2-undecyltridecanedioic acid and (S)-2-((39-((2,5-dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate ent-F1-Peak1-Peg12 and Intermediate ent-F1-Peak2-Peg12)

### Step 4-1: Intermediate ent-F1-Peak1-Peg12

A mixture of the crude residue containing Intermediate ent-F1-Peak1-Peg12-3 (5.97 g, 4.52 mmol) and MgSO₄ (0.544 g, 4.52 mmol) in THF (50 mL) was degassed with N₂. To the mixture was added Pd/C (10 wt. %, 481 mg, 0.452 mmol) and the mixture was purged with H₂ and allowed to stir at RT for 18 h. The mixture was filtered through Celite®, washed with MeOH, and the filtrate was concentrated under reduced pressure providing the crude title compound Intermediate ent-F1-Peak1-Peg12 (6.22 g) which was used without further purification. LCMS Method T11: Rt=0.94 min; MS m/z [M+H]⁺=1140.7.

### Step 4-2: Intermediate ent-F1-Peak2-Peg12

A mixture of the crude residue containing Intermediate ent-F1-Peak2-Peg12-3 (5.97 g, 4.52 mmol) and MgSO₄ (0.544 g, 4.52 mmol) in THF (50 mL) was degassed with N₂. To the mixture was added Pd/C (10 wt. %, 481 mg, 0.452 mmol) and the mixture was purged with H₂ and allowed to stir at RT for 18 h. The mixture was filtered through Celite®, washed with MeOH, and the filtrate was concentrated under reduced pressure providing the crude title compound Intermediate ent-F1-Peak2-Peg12 (6.16 g) which was used without further purification. LCMS Method T11: Rt=0.94 min; MS m/z [M+H]⁺=1140.7.

### Example 28d: 2-((75-((2,5-Dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)-2-undecyltridecanedioic acid (Intermediate rac-F1-Peg24)

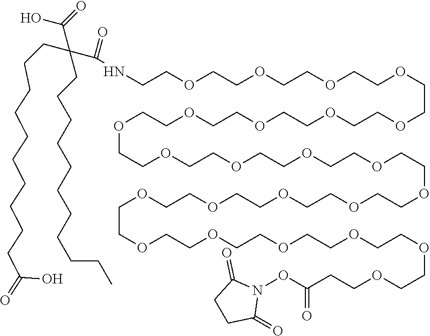 [SMILES: CCCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O]

### Step 1: 14-((Benzyloxy)carbonyl)-3,15-dioxo-1-phenyl-14-undecyl-2,19,22,25,28,31,34,37,40,43,46,49,52,55,58,61,64,67,70,73,76,79,82,85,88-pentacosaoxa-16-azahennonacontan-91-oic acid (Intermediate rac-F1-Peg24-1)

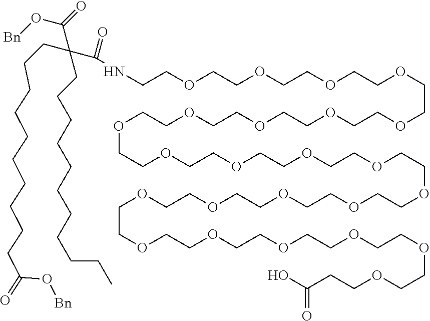 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)O)C(=O)OCc1ccccc1]

Step 1a: To a flask were added 13-(benzyloxy)-2-((benzyloxy)carbonyl)-13-oxo-2-undecyltridecanoic acid (Intermediate rac-F1, 640 g, 1.03 mol), DCM (8.3 kg), and DMF (3 g). This mixture was stirred at 25° C. and oxalyl chloride (170 g, 1.34 mol) was added dropwise. The RM was stirred for another 2 to 3 h. Concentration of the RM and solvent swap with heptane gave a crude mixture of the active acyl chloride (691 g) to which DCM (8.5 kg) was added to form a solution and was used directly in the next step.

Step 1b: To a flask was added 1-amino-3, 6, 9, 12, 15, 18, 21, 24, 27, 30, 33, 36, 39, 42, 45, 48, 51, 54, 57, 60, 63, 66, 69, 72-tetracosaoxapentaheptacontan-75-oic acid (900 g, 0.79 mol), DCM (6.0 kg), and DIPEA (203 g, 1.57 mol). This mixture was stirred at 25° C. followed by dropwise addition of the crude acyl chloride solution from Step 1a (6.76 kg, 0.75 mol, 7.1% pure). The RM was stirred for another 1-2 h and then acidic resin (1.3 kg) was added. After stirring for 30 min, the mixture was filtered and MgSO₄ (1.3 kg) added to the filtrate. Stirring was continued for 30 min and then filtered. The filtrate was concentrated under reduced pressure to provide a crude residue. The residue was purified with Al₂O₃ with mobile phase including MTBE, DCM, MeOH to provide the title compound Intermediate rac-F1-Peg24-1 (960 g). ¹H NMR (400 MHz, Chloroform-d) δ [ppm] 0.86-0.93 (m, 3H), 0.93-1.04 (m, 2H), 1.19 (br s, 15H), 1.23-1.37 (m, 15H), 1.61-1.68 (m, 2H), 1.78 (td, J=12.44, 4.34 Hz, 2H) 1.92-2.05 (m, 2H), 2.37 (t, J=7.58 Hz, 2H), 2.62 (t, J=6.05 Hz, 2H), 3.49 (dd, J=6.72, 2.32 Hz, 2H), 3.52-3.59 (m, 2H), 3.59-3.73 (m, 92H), 3.80 (t, J=6.05 Hz, 2H), 5.13 (s, 2H), 5.18 (s, 2H), 7.31-7.42 (m, 10H), 8.09 (t, J=5.26 Hz, 1H). LCMS Method W11: Rt=15.75 min/18.0 min, MS m/z [M+H]⁺ 1751.1.

### Step 2: 77,87-Dibenzyl 1-(2,5-dioxopyrrolidin-1-yl) 76-oxo-77-undecyl-3,6,9,12,15,18,21,24,27,30,33,36,39,42,45,48,51,54,57,60,63,66,69,72-tetracosaoxa-75-azaheptaoctacontane-1,77,87-tricarboxylate (Intermediate rac-F1-Peg24-2)

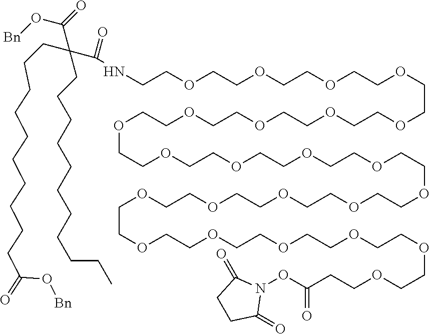 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O)C(=O)OCc1ccccc1]

To the solution of 14-((benzyloxy)carbonyl)-3,15-dioxo-1-phenyl-14-undecyl-2,19,22,25,28,31,34,37,40,43,46,49,52,55,58,61,64,67,70,73,76,79,82,85,88-pentacosaoxa-16-azahennonacontan-91-oic acid (Intermediate rac-F1-Peg24-1, 920 g, 0.53 mol) in DCM (6.1 kg) was added TEA (11 g, 0.11 mol). This mixture was stirred to provide a clear solution, then DSC (161 g, 0.63 mol) was added and the mixture was stirred at 25° C. for 2 h. Acidic resin (180 g) was added and this mixture was stirred for the next 30 min, then MgSO₄ (180 g) was added into the mixture and stirred for 30 min. The mixture was filtered to provide a clear light yellow solution. Concentration of the filtrate under reduced pressure gave crude Intermediate rac-F1-Peg24-2 which was used directly in the next step. LCMS Method W11: Rt=15.75 min/18.0 min, MS m/z [M+H]+ 1848.2.

### Step 3: 2-((75-((2,5-Dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)-2-undecyltridecanedioic acid (Intermediate rac-F1-Peg24)

To a hydrogenation reactor was added 77,87-dibenzyl 1-(2,5-dioxopyrrolidin-1-yl) 76-oxo-77-undecyl-3,6,9,12,15,18,21,24,27,30,33,36,39,42,45,48,51,54,57,60,63,66,69,72-tetracosaoxa-75-azaheptaoctacontane-1,77,87-tricarboxylate (Intermediate rac-F1-Peg24-2, 986 g, 0.48 mol, 90% pure), THF (7.6 kg), and Pd/C (10 wt. %, 110 g). Then MgSO₄ (110 g) was added. The reaction was purged with N₂ and then placed under an H₂ atmosphere. The mixture was stirred at 25° C. for 3-24 h. After complete consumption of starting material, more MgSO₄ (220 g) was added and the RM stirred for an additional 30 min and filtered. The filter cake was washed with 100 mL THF. The filtrate and THF wash were combined and concentrated under reduced pressure to provide the title compound Intermediate rac-F1-Peg24 (800 g). ¹H NMR (400 MHz, Chloroform-d) δ [ppm] 0.84-0.94 (m, 3H), 1.17 (br s, 2H), 1.21-1.39 (m, 30H), 1.57-1.68 (m, 2H), 1.69-1.80 (m, 2H), 1.97-2.10 (m, 2H), 2.34 (t, J=7.21 Hz, 2H), 2.86 (s, 4H), 2.92 (t, J=6.48 Hz, 2H), 3.51-3.73 (m, 96H), 3.87 (t, J=6.48 Hz, 2H), 7.45 (t, J=4.46 Hz, 1H). LCMS Method W11: Rt=14.00 min/18.0 min, MS m/z [M+H]+ 1668.0.

### Example 29d: 15-((39-((2,5-Dioxopyrrolidin-1-yl)oxy)-39-oxo-3,6,9,12,15,18,21,24,27,30,33,36-dodecaoxanonatriacontyl)carbamoyl)nonacosane-1,15,29-tricarboxylic acid (Intermediate F2-Peg12)

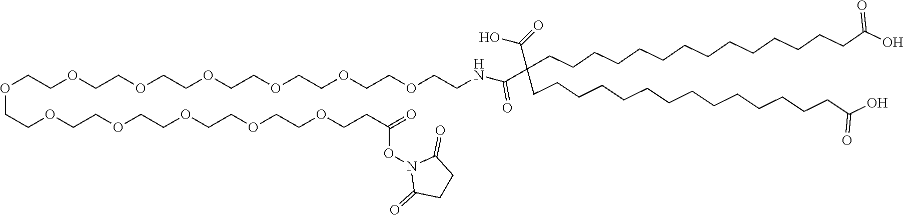

### Step 1: 1,15,29-Tribenzyl 15-(2,5-dioxopyrrolidin-1-yl) nonacosane-1,15,15,29-tetracarboxylate (Intermediate F2-Peg12-1)

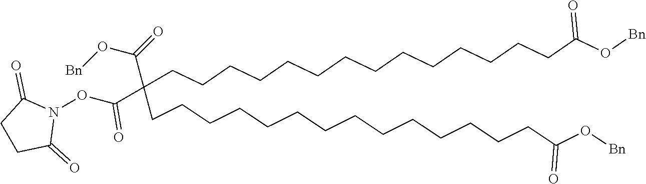 [SMILES: O=C(CCCCCCCCCCCCCCC(CCCCCCCCCCCCCCC(=O)OCc1ccccc1)(C(=O)OCc1ccccc1)C(=O)ON1C(=O)CCC1=O)OCc1ccccc1]

To a solution of crude 17-(benzyloxy)-2-(15-(benzyloxy)-15-oxopentadecyl)-2-((benzyloxy)carbonyl)-17-oxoheptadecanoic acid (Intermediate F2, 81.6 g) and 1-hydroxypyrrolidine-2,5-dione (13.1 g, 114 mmol) in DCM (800 mL) was added DCC (1M in DCM, 143 mL). The mixture was stirred at 20° C. for 12 h. The RM was filtered and concentrated under reduced pressure. The resulting residue was purified by column chromatography (SiO₂, eluting with PE/Et₂O=1/0 to 5/1) affording the title compound Intermediate F2-Peg12-1 (44.1 g) as a white solid. ¹H NMR (400 MHz, Chloroform-d) δ [ppm] 7.30-7.44 (m, 15H), 5.24 (s, 2H), 5.12 (s, 4H), 2.82 (d, J=2.0 Hz, 4H), 2.36 (t, J=7.6 Hz, 4H), 1.95-2.04 (m, 4H), 1.59-1.70 (m, 4H), 1.18-1.36 (m, 44H). LCMS Method C3: Rt=1.24 min, MS m/z [M+Na]⁺=974.6.

### Step 2: 18-(15-(Benzyloxy)-15-oxopentadecyl)-18-((benzyloxy)carbonyl)-3,19-dioxo-1-phenyl-2,23,26,29,32,35,38,41,44,47,50,53,56-tridecaoxa-20-azanonapentacontan-59-oic acid (Intermediate F2-Peg12-2)

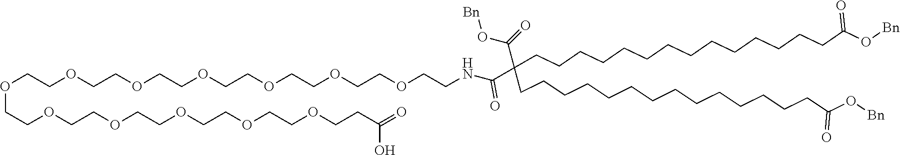

To a solution of 1,15,29-tribenzyl 15-(2,5-dioxopyrrolidin-1-yl) nonacosane-1,15,15,29-tetracarboxylate (Intermediate F2-Peg12-1, 20.0 g, 20.9 mmol), 1-amino-3,6,9,12,15,18,21,24,27,30,33,36-dodecaoxanonatriacontan-39-oic acid (14.2 g, 23.0 mmol) and DMAP (256 mg, 2.10 mmol) in DCM (200 mL) was added DIPEA (4.06 g, 31.4 mmol). The mixture was stirred at 25° C. for 12 h. The RM was washed with aq. HCl (0.5M, 2×50 mL), and brine (50 mL), dried over Na₂SO₄, filtered and concentrated under reduced pressure providing the crude title compound Intermediate F2-Peg12-2 (25.8 g) as a yellow oil which was directly used in the next step without further purification. ¹H NMR (400 MHz, Chloroform-d) δ [ppm] 7.29-7.40 (m, 15H), 5.16 (s, 2H), 5.11 (s, 4H), 3.79-3.71 (m, 2H), 3.59-3.69 (m, 44H), 3.52-3.56 (m, 2H), 3.45-3.51 (m, 2H), 2.56 (s, 2H), 2.35 (t, J=7.6 Hz, 4H), 1.90-2.03 (m, 2H), 1.71-1.83 (m, 2H), 1.58-1.69 (m, 4H), 1.12-1.36 (m, 44H). LCMS Method C3: Rt=1.19 min, MS m/z [M+H]+=1455.0.

### Step 3: 41,55-Dibenzyl 1-(2,5-dioxopyrrolidin-1-yl) 41-(15-(benzyloxy)-15-oxopentadecyl)-40-oxo-3,6,9,12,15,18,21,24,27,30,33,36-dodecaoxa-39-azapentapentacontane-1,41,55-tricarboxylate (Intermediate F2-Peg12-3)

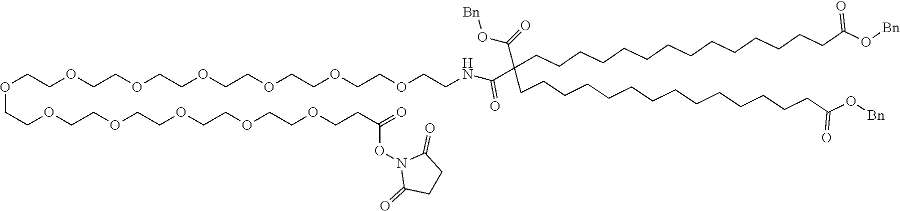 [SMILES: O=C(CCCCCCCCCCCCCCC(CCCCCCCCCCCCCCC(=O)OCc1ccccc1)(C(=O)NCCOCCOCCOCCOCCOCCOCCOCCOCCOCCOCCOCCOCCC(=O)ON1C(=O)CCC1=O)C(=O)OCc1ccccc1)OCc1ccccc1]

To a solution of crude 18-(15-(benzyloxy)-15-oxopentadecyl)-18-((benzyloxy)carbonyl)-3,19-dioxo-1-phenyl-2,23,26,29,32,35,38,41,44,47,50,53,56-tridecaoxa-20-azanonapentacontan-59-oic acid (Intermediate F2-Peg12-2, 25.8 g) and 1-hydroxypyrrolidine-2,5-dione (2.45 g, 21.2 mmol) in DCM (250 mL) was added DCC (1 M, 26.6 mL). The mixture was stirred at 25° C. for 12 h. The RM was diluted with EtOAc (500 mL), filtered and concentrated under reduced pressure. The residue was purified by column chromatography (SiO₂, eluting with DCM/MeOH=100/1 to 20/1) providing the title compound Intermediate F2-Peg12-3 (18.5 g) as a yellow oil. LCMS Method C3: Rt=1.19 min, MS m/z [M+H]⁺=1552.8.

### Step 4: 15-((39-((2,5-Dioxopyrrolidin-1-yl)oxy)-39-oxo-3,6,9,12,15,18,21,24,27,30,33,36-dodecaoxanonatriacontyl)carbamoyl)nonacosane-1,15,29-tricarboxylic acid (Intermediate F2-Peg12)

To a solution of 41,55-dibenzyl 1-(2,5-dioxopyrrolidin-1-yl) 41-(15-(benzyloxy)-15-oxopentadecyl)-40-oxo-3,6,9,12,15,18,21,24,27,30,33,36-dodecaoxa-39-azapentapentacontane-1,41,55-tricarboxylate (Intermediate F2-Peg12-3, 18.5 g, 11.9 mmol) in THF (150 mL) was added Pd/C (1.27 g, 1.19 mmol, 10 wt. %) and Pd(OH)₂/C (838 mg, 1.19 mmol, 20 wt. %) under N₂ atmosphere. The atmosphere was replaced with H₂ from a balloon reservoir and the mixture was stirred at 25° C. for 12 h. The RM was filtered and concentrated under reduced pressure. The resulting residue was purified by column chromatography (SiO₂, eluting with DCM/MeOH=1/0 to 20/1) providing the title compound Intermediate F2-Peg12 (11.3 g) as a white gum. ¹H NMR (400 MHz, Chloroform-d) δ [ppm] 3.86 (t, J=6.6 Hz, 2H), 3.61-3.71 (m, 44H), 3.55-3.61 (m, 2H), 3.49-3.55 (m, 2H), 2.91 (t, J=6.6 Hz, 2H), 2.81-2.88 (m, 4H), 2.34 (t, J=7.6 Hz, 4H), 1.96-2.07 (m, 2H), 1.69-1.80 (m, 2H), 1.63 (m, 4H), 1.19-1.38 (m, 44H). LCMS Method C3: Rt=0.95 min, MS m/z [M+H]⁺=1281.7.

### Example 30d: 43-((2,5-Dioxopyrrolidin-1-yl)oxy)-3,43-dioxo-2,2-bis(14-phosphonotetradecyl)-7,10,13,16,19,22,25,28,31,34,37,40-dodecaoxa-4-azatritetracontanoic acid (Intermediate F3-Peg12)

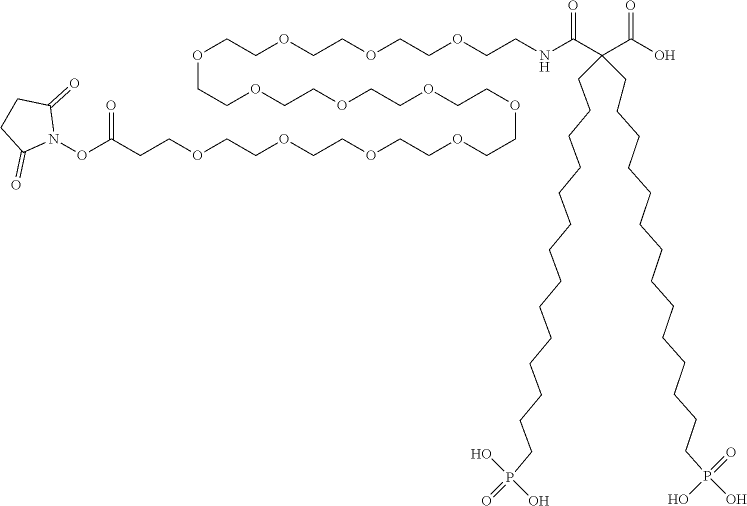 [SMILES: O=C(CCOCCOCCOCCOCCOCCOCCOCCOCCOCCOCCOCCOCCNC(=O)C(CCCCCCCCCCCCCCP(=O)(O)O)(CCCCCCCCCCCCCCP(=O)(O)O)C(=O)O)ON1C(=O)CCC1=O]

### Step 1: 4,4-Bis(14-(bis(benzyloxy)phosphoryl)tetradecyl)-3,5-dioxo-1-phenyl-2,9,12,15,18,21,24,27,30,33,36,39,42-tridecaoxa-6-azapentatetracontan-45-oic acid (Intermediate F3-Peg12-1)

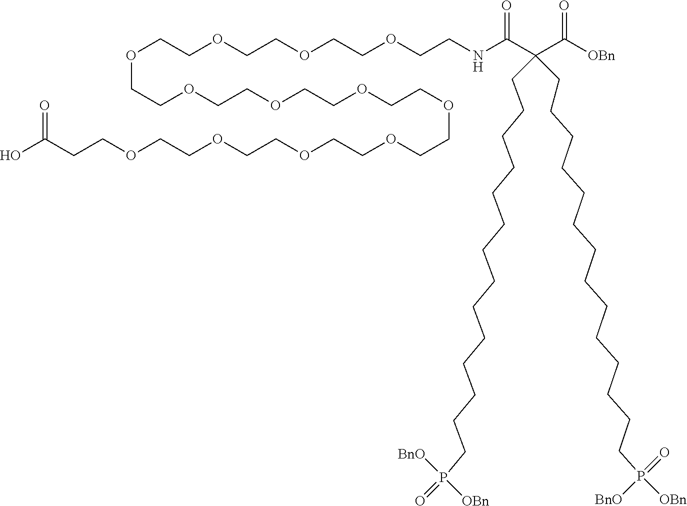 [SMILES: O=C(O)CCOCCOCCOCCOCCOCCOCCOCCOCCOCCOCCOCCOCCNC(=O)C(CCCCCCCCCCCCCCP(=O)(OCc1ccccc1)OCc1ccccc1)(CCCCCCCCCCCCCCP(=O)(OCc1ccccc1)OCc1ccccc1)C(=O)OCc1ccccc1]

A mixture of 1-benzyl 3-(2,5-dioxopyrrolidin-1-yl) 2,2-bis(14-(bis(benzyloxy)phosphoryl)tetradecyl)malonate (Intermediate F3, 713 mg, 0.592 mmol), 1-amino-3,6,9,12,15,18,21,24,27,30,33,36-dodecaoxanonatriacontan-39-oic acid (452 mg, 0.732 mmol), DIPEA (258 uL, 1.480 mmol) and DMAP (7.23 mg, 0.059 mmol) in DCM (6 mL) was stirred for 16 h. Additional 1-amino-3,6,9,12,15,18,21,24,27,30,33,36-dodecaoxanonatriacontan-39-oic acid (34 mg, 0.028 mmol) was added and stirring was continued for 4 h. The mixture was concentrated under reduced pressure and the resulting residue purified by normal phase column chromatography (Isco Gold SiO₂ cartridge, eluting with 0 to 10% MeOH in DCM) to yield the title compound Intermediate F3-Peg12-1 (781 mg). LCMS Method T7: Rt=1.60 min; MS m/z [M+2H]²⁺=854.6.

### Step 2: 1-Benzyl 43-(2,5-dioxopyrrolidin-1-yl) 2,2-bis(14-(bis(benzyloxy)phosphoryl)tetradecyl)-3-oxo-7,10,13,16,19,22,25,28,31,34,37,40-dodecaoxa-4-azatritetracontanedioate (Intermediate F3-Peg12-2)

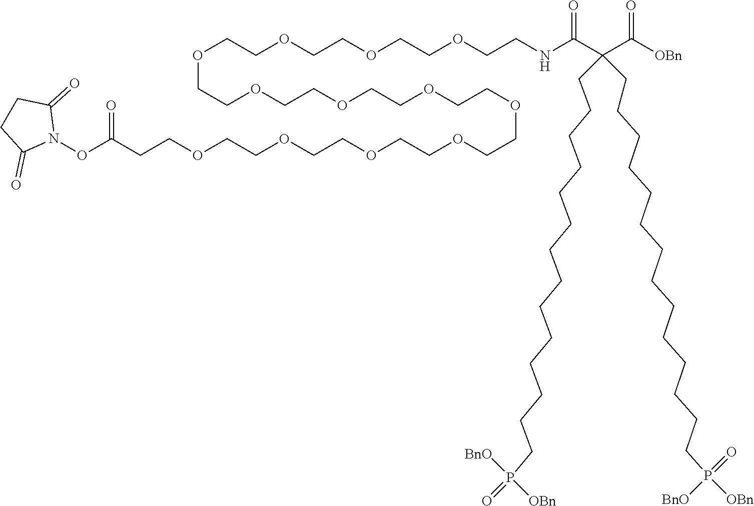 [SMILES: O=C(CCOCCOCCOCCOCCOCCOCCOCCOCCOCCOCCOCCOCCNC(=O)C(CCCCCCCCCCCCCCP(=O)(OCc1ccccc1)OCc1ccccc1)(CCCCCCCCCCCCCCP(=O)(OCc1ccccc1)OCc1ccccc1)C(=O)OCc1ccccc1)ON1C(=O)CCC1=O]

To 4,4-bis(14-(bis(benzyloxy)phosphoryl)tetradecyl)-3,5-dioxo-1-phenyl-2,9,12,15,18,21,24,27,30,33,36,39,42-tridecaoxa-6-azapentatetracontan-45-oic acid (Intermediate F3-Peg12-1, 5.3 g, 3.10 mmol) and N-hydroxysuccinimide (393 mg, 3.42 mmol) in anhydrous DCM (12 mL) was added a solution of DCC in DCM (1M, 3.26 mL). After stirring for 16 h the mixture was directly purified by column chromatography over SiO₂ (eluting with 0 to 10% MeOH/DCM) to yield the title compound Intermediate F3-Peg12-2 (3.25 g) as a clear oil. LCMS Method T11: Rt=1.54 min, MS m/z [M+2H]²⁺=902.8.

### Step 3: 43-((2,5-Dioxopyrrolidin-1-yl)oxy)-3,43-dioxo-2,2-bis(14-phosphonotetradecyl)-7,10,13,16,19,22,25,28,31,34,37,40-dodecaoxa-4-azatritetracontanoic acid (Intermediate F3-Peg12)

1-Benzyl 43-(2,5-dioxopyrrolidin-1-yl) 2,2-bis(14-(bis(benzyloxy)phosphoryl)tetradecyl)-3-oxo-7,10,13,16,19,22,25,28,31,34,37,40-dodecaoxa-4-azatritetracontanedioate (Intermediate F3-Peg12-2, 3.25 g, 1.80 mmol) was dissolved in anhydrous THF (18 mL) and dry Pd/C (10 wt. %, 959 mg, 0.901 mmol) was added. The mixture was stirred under H₂ atmosphere (balloon) for 16 h. The RM was diluted with anhydrous DCM (9 mL) and filtered through a glass microfiber filter. The resulting filtrate was concentrated under reduced pressure to yield the crude title compound Intermediate F3-Peg12 (1.98 g) as a waxy semisolid which was used without further purification. LCMS Method T7: Rt=1.22 min, MS m/z [M−H]⁻=1352.1.

### Example 31d: ((S)-20-carboxy-1,17,22-trioxo-1-(perfluorophenoxy)-21-undecyl-4,7,10,13-tetraoxa-16,21-diazadotriacontan-32-oic acid (Intermediate F4-Peg4)

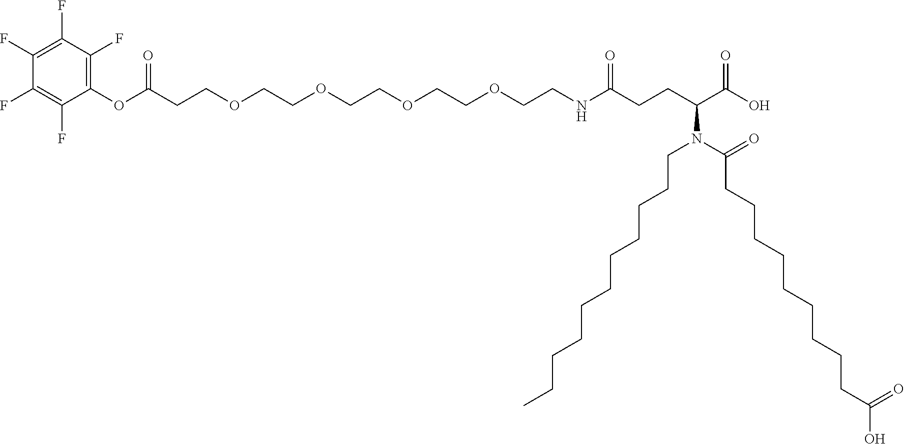 [SMILES: CCCCCCCCCCCN(C(=O)CCCCCCCCCC(=O)O)[C@@H](CCC(=O)NCCOCCOCCOCCOCCC(=O)Oc1c(F)c(F)c(F)c(F)c1F)C(=O)O]

### Step 1: 19,30-Dibenzyl 1-(perfluorophenyl) (S)-16,21-dioxo-20-undecyl-3,6,9,12-tetraoxa-15,20-diazatriacontane-1,19,30-tricarboxylate (Intermediate F4-Peg4-1)

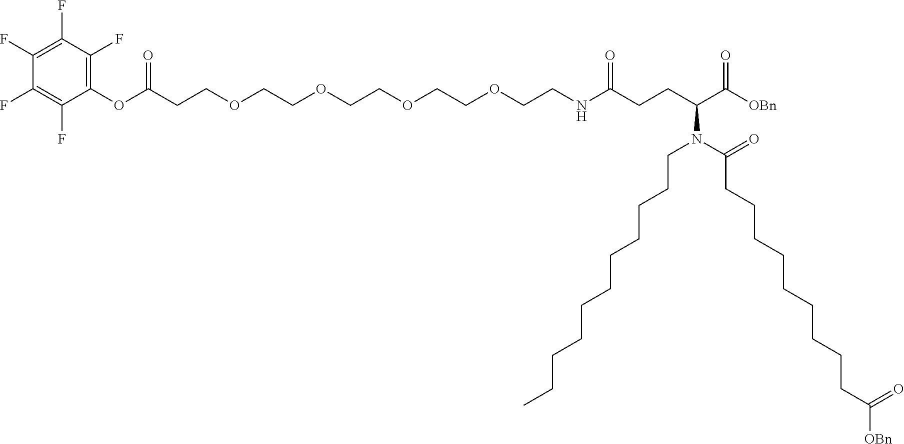 [SMILES: CCCCCCCCCCCN(C(=O)CCCCCCCCCC(=O)OCc1ccccc1)[C@@H](CCC(=O)NCCOCCOCCOCCOCCC(=O)Oc1c(F)c(F)c(F)c(F)c1F)C(=O)OCc1ccccc1]

To a solution of 1-benzyl 5-(perfluorophenyl) N-(11-(benzyloxy)-11-oxoundecanoyl)-N-undecyl-L-glutamate (Intermediate F4-S, 7.44 g, 8.73 mmol) and 1-amino-3,6,9,12-tetraoxapentadecan-15-oic acid (2.24 g, 8.29 mmol) in anhydrous DCM (120 mL) was added DIPEA (7.62 mL, 43.7 mmol) and the RM stirred at RT for 1 h. Bis(pentafluorophenyl)carbonate (3.62 g, 8.90 mmol) was added and stirring continued for a further 1 h at RT. The RM was then concentrated under reduced pressure and the crude oily residue was re-dissolved in DIPE (70 mL). Heptane was added to the solution until it became cloudy and emulsified. The mixture was cooled over dry ice and the supernatant decanted. The remaining oily residue was concentrated under reduced pressure to give the title compound Intermediate F4-Peg4-1 (7.96 g) as clear, golden orange oil. LCMS Method W6: Rt=1.76 min; MS m/z [M+NH₄]⁺=1110.6.

### Step 2: ((S)-20-Carboxy-1,17,22-trioxo-1-(perfluorophenoxy)-21-undecyl-4,7,10,13-tetraoxa-16,21-diazadotriacontan-32-oic acid (Intermediate F4-Peg4)

A suspension of 19,30-dibenzyl 1-(perfluorophenyl) (S)-16,21-dioxo-20-undecyl-3,6,9,12-tetraoxa-15,20-diazatriacontane-1,19,30-tricarboxylate (Intermediate F4-Peg4-1, 5.26 g, 4.57 mmol) and Pd/C (10 wt. %, [BASF 4505 D/R E], 0.972 g, 0.914 mmol) in anhydrous THF (60 mL) was agitated under a 0.1 bar H₂ for 45 min at RT. The RM was filtered and the collected solids washed with anhydrous THF (20 mL). The filtrate was passed through an Agilent Stratospheres PL-Thiol MP cartridge (5 g) and the cartridge washed with anhydrous THF (30 mL). The filtrate was then concentrated under reduced pressure and the oily product triturated ultrasonically with DIPE/heptane (1/1, 2×80 mL). After cooling over dry ice, the supernatants were decanted and the remaining oily residue concentrated under reduced pressure to give the title compound Intermediate F4-Peg4 as a clear, golden yellow oil (3.37 g). LCMS Method W6: Rt=1.45 min; MS m/z [M+NH₄]+=930.5.

### Example 32d: (S)-32-Carboxy-1,29,34-trioxo-1-(perfluorophenoxy)-33-undecyl-4,7,10,13,16,19,22,25-octaoxa-28,33-diazatetratetracontan-44-oic acid (Intermediate F4-Peg8)

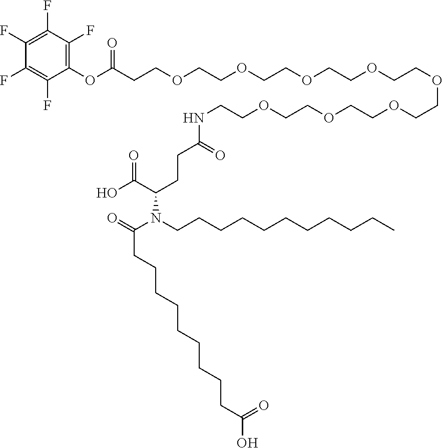 [SMILES: CCCCCCCCCCCN(C(=O)CCCCCCCCCC(=O)O)[C@@H](CCC(=O)NCCOCCOCCOCCOCCOCCOCCOCCOCCC(=O)Oc1c(F)c(F)c(F)c(F)c1F)C(=O)O]

### Step 1: 31,42-Dibenzyl 1-(perfluorophenyl) (S)-28,33-dioxo-32-undecyl-3,6,9,12,15,18,21,24-octaoxa-27,32-diazadotetracontane-1,31,42-tricarboxylate (Intermediate F4-Peg8-1)

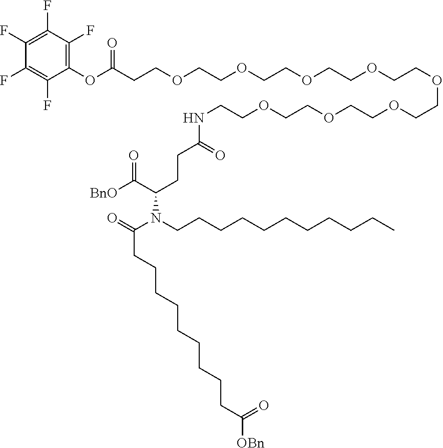 [SMILES: CCCCCCCCCCCN(C(=O)CCCCCCCCCC(=O)OCc1ccccc1)[C@@H](CCC(=O)NCCOCCOCCOCCOCCOCCOCCOCCOCCC(=O)Oc1c(F)c(F)c(F)c(F)c1F)C(=O)OCc1ccccc1]

To a solution of 1-benzyl 5-(perfluorophenyl) N-(11-(benzyloxy)-11-oxoundecanoyl)-N-undecyl-L-glutamate (Intermediate F4-S, 1.76 g, 2.07 mmol) and 1-amino-3,6,9,12,15,18,21,24-octaoxaheptacosan-27-oic acid (0.934 g, 2.03 mmol) in anhydrous DCM (20 mL) was added DIPEA (1.81 mL, 10.36 mmol) and the RM stirred at RT for 1 h. Bis(pentafluorophenyl)carbonate (0.898 g, 2.28 mmol) was added and stirring continued for a further 1 h at RT. The RM was then concentrated under reduced pressure and the crude oily residue was triturated ultrasonically with heptane (2×30 mL) and heptane/DIPE (1/1, 40 mL). The mixture was cooled over dry ice and the supernatant decanted. The remaining oily residue was concentrated under reduced pressure to give the title compound Intermediate F4-Peg8-1 as clear, golden yellow oil (2.61 g). LCMS Method W6: Rt=1.75 min; MS m/z [M−H−Pfp]⁻=1101.9.

### Step 2: (S)-32-Carboxy-1,29,34-trioxo-1-(perfluorophenoxy)-33-undecyl-4,7,10,13,16,19,22,25-octaoxa-28,33-diazatetratetracontan-44-oic acid (Intermediate F4-Peg8)

A suspension of 31,42-dibenzyl 1-(perfluorophenyl) (S)-28,33-dioxo-32-undecyl-3,6,9,12,15,18,21,24-octaoxa-27,32-diazadotetracontane-1,31,42-tricarboxylate (Intermediate F4-Peg8-1, 2.61 g, 1.95 mmol) and Pd/C (10 wt. %, [BASF 4505 D/R E], 0.416 g, 0.391 mmol) in anhydrous THF (30 mL) was agitated under a 0.1 bar H₂ for 45 min at RT. The RM was filtered and the collected solids washed with anhydrous THF (20 mL). The filtrate was passed through an Agilent Stratospheres PL-Thiol MP cartridge (500 mg) and the cartridge washed with anhydrous THF (10 mL). The filtrate was then concentrated under reduced pressure and the oily product ultrasonically triturated with DIPE (2×40 mL). After cooling the mixture to 4° C., the supernatants were decanted and the remaining oily residue concentrated under reduced pressure to give the title compound Intermediate F4-Peg8 (1.63 g) as a clear, colorless oil. LCMS Method W6: Rt=1.44 min; MS m/z [M−H]⁻=1087.6.

### Example 33d: (S)-44-Carboxy-1,41,46-trioxo-1-(perfluorophenoxy)-45-undecyl-4,7,10,13,16,19,22,25,28,31,34,37-dodecaoxa-40,45-diazahexapentacontan-56-oic acid (Intermediate F4-Peg12-S)

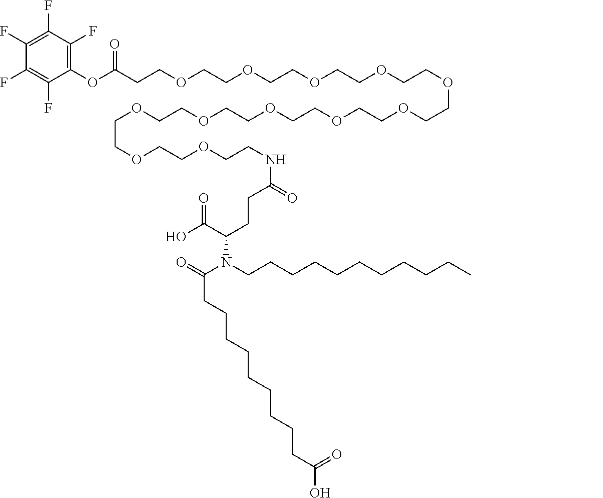 [SMILES: CCCCCCCCCCCN(C(=O)CCCCCCCCCC(=O)O)[C@@H](CCC(=O)NCCOCCOCCOCCOCCOCCOCCOCCOCCOCCOCCOCCOCCC(=O)Oc1c(F)c(F)c(F)c(F)c1F)C(=O)O]

### Step 1: 43,54-Dibenzyl 1-(perfluorophenyl) (S)-40,45-dioxo-44-undecyl-3,6,9,12,15,18,21,24,27,30,33,36-dodecaoxa-39,44-diazatetrapentacontane-1,43,54-tricarboxylate (Intermediate F4-Peg12-S-1)

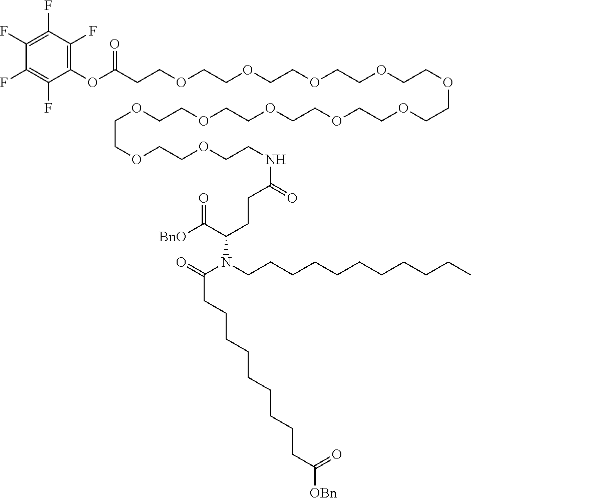 [SMILES: CCCCCCCCCCCN(C(=O)CCCCCCCCCC(=O)OCc1ccccc1)[C@@H](CCC(=O)NCCOCCOCCOCCOCCOCCOCCOCCOCCOCCOCCOCCOCCC(=O)Oc1c(F)c(F)c(F)c(F)c1F)C(=O)OCc1ccccc1]

To a solution of 1-amino-3,6,9,12,15,18,21,24,27,30,33,36-dodecaoxanonatriacontan-39-oic acid (19.9 g, 31.8 mmol) and DIPEA (17.0 mL, 97 mmol) in anhydrous DCM (120 mL) was added a solution of 1-benzyl 5-(perfluorophenyl) N-(11-(benzyloxy)-11-oxoundecanoyl)-N-undecyl-L-glutamate (Intermediate F4-S, 27.6 g, 32.5 mmol) in anhydrous DCM (120 mL). The RM was stirred at RT for 2 h and then bis(pentafluorophenyl)carbonate (14.5 g, 35.7 mmol) was added. Stirring was continued for a further 2 h at RT and then the RM was concentrated under reduced pressure. The crude material was re-dissolved in DCM (200 mL) and the solution transferred to a separatory funnel. The organic layer was washed with 1N HCl (2×150 mL), brine (120 mL), dried over MgSO₄, filtered and concentrated under reduced pressure to give the title compound Intermediate F4-Peg12-S-1 (62.08 g) as a dark orange oil, which was used directly in the next step. LCMS Method W6: Rt=1.69 min; MS m/z [M+NH₄]⁺=1463.1.

### Step 2: (S)-44-Carboxy-1,41,46-trioxo-1-(perfluorophenoxy)-45-undecyl-4,7,10,13,16,19,22,25,28,31,34,37-dodecaoxa-40,45-diazahexapentacontan-56-oic acid (Intermediate F4-Peg12-S)

A suspension of 43,54-dibenzyl 1-(perfluorophenyl) (S)-40,45-dioxo-44-undecyl-3,6,9,12,15,18,21,24,27,30,33,36-dodecaoxa-39,44-diazatetrapentacontane-1,43,54-tricarboxylate (Intermediate F4-Peg12-S-1, 49.8 g, 25.8 mmol) and Pd/C (10 wt. %, [BASF 4505 D/R E], 3.02 g, 2.84 mmol) in anhydrous THF (250 mL) was agitated under a 0.1 bar H₂ for 4 h at RT. The RM was filtered and the collected solids washed with anhydrous THF (100 mL). The combined filtrate and THF wash were partially concentrated under reduced pressure until approx. 150 mL of the solution remained. This solution was then passed sequentially through two Agilent Stratospheres PL-Thiol MP cartridges (2×5 g). After washing the cartridges with anhydrous THF (3×50 mL), the combined filtrate and THF washes were concentrated under reduced pressure to give a clear, almost colorless oil. The oily product was then ultrasonically triturated with warm heptane (3×100 mL). After cooling to 4° C., the supernatants were decanted and the remaining oily residue concentrated under reduced pressure to give a clear, pale golden-yellow oil. The oil was then dissolved in Et₂O (120 mL) and enough heptane added such that the solution became cloudy and emulsified. The mixture was left at 4° C. for 2-3 h by which time solids had precipitated. The supernatant was then decanted and the remaining oily solid concentrated under reduced pressure to give the title compound Intermediate F4-Peg12-S as a clear, pale straw-colored oil (35.5 g). The obtained material was stored under inert atmosphere between 0° C. and −20° C. until use*. LCMS Method W6: Rt=1.45 min; MS m/z [M+NH₄]+=1283.0. *We believe that Intermediate F4-Peg12-S epimerizes upon storage over time. Racemization can be minimized by preparing and using it immediately after the product has been prepared.

### Example 34d: (R)-44-Carboxy-1,41,46-trioxo-1-(perfluorophenoxy)-45-undecyl-4,7,10,13,16,19,22,25,28,31,34,37-dodecaoxa-40,45-diazahexapentacontan-56-oic acid (Intermediate F4-Peg12-R)

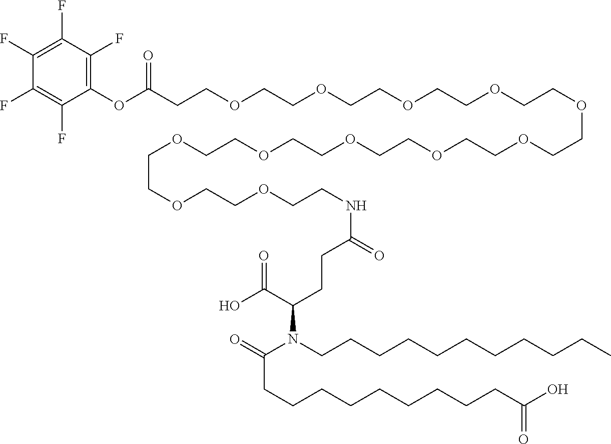 [SMILES: CCCCCCCCCCCN(C(=O)CCCCCCCCCC(=O)O)[C@H](CCC(=O)NCCOCCOCCOCCOCCOCCOCCOCCOCCOCCOCCOCCOCCC(=O)Oc1c(F)c(F)c(F)c(F)c1F)C(=O)O]

### Step 1a,b: 43,54-Dibenzyl 1-(perfluorophenyl) (R)-40,45-dioxo-44-undecyl-3,6,9,12,15,18,21,24,27,30,33,36-dodecaoxa-39,44-diazatetrapentacontane-1,43,54-tricarboxylate (Intermediate F4-Peg12-R-1)

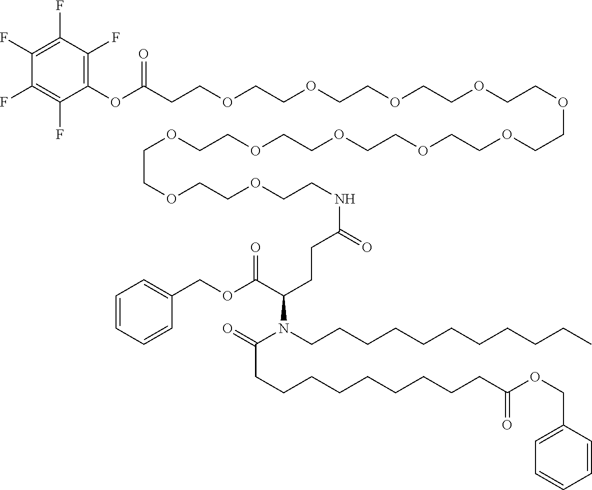 [SMILES: CCCCCCCCCCCN(C(=O)CCCCCCCCCC(=O)OCc1ccccc1)[C@H](CCC(=O)NCCOCCOCCOCCOCCOCCOCCOCCOCCOCCOCCOCCOCCC(=O)Oc1c(F)c(F)c(F)c(F)c1F)C(=O)OCc1ccccc1]

To a solution of HOOC-dPEG12-NH₂ (0.730 g, 1.17 mmol) and DIPEA (0.619 mL, 3.55 mmol) in anhydrous DCM (4 mL) was added a solution of 1-benzyl 5-(perfluorophenyl) N-(11-(benzyloxy)-11-oxoundecanoyl)-N-undecyl-D-glutamate (Intermediate F4-R, 1 g, 1.18 mmol) in anhydrous DCM (4 mL). The RM was stirred at RT for 2 h and then bis(pentafluorophenyl)carbonate (0.528 g, 1.30 mmol) was added. Stirring was continued for a additional 2 h at RT and then the RM washed with aq. HCl solution (5N, 3×10 mL), sat. aq. brine (10 mL), dried over MgSO₄, filtered and concentrated under reduced pressure to give the title compound Intermediate F4-Peg12-R-1 (1.934 g) as a colorless oil. LCMS Method W5: Rt=1.57 min; m/z [M+H+NH₄]²⁺=732.0.

### Step 2: (R)-44-Carboxy-1,41,46-trioxo-1-(perfluorophenoxy)-45-undecyl-4,7,10,13,16,19,22,25,28,31,34,37-dodecaoxa-40,45-diazahexapentacontan-56-oic acid (Intermediate F4-Peg12-R)

A suspension of 43,54-dibenzyl 1-(perfluorophenyl) (R)-40,45-dioxo-44-undecyl-3,6,9,12,15,18,21,24,27,30,33,36-dodecaoxa-39,44-diazatetrapentacontane-1,43,54-tricarboxylate (Intermediate F4-Peg12-R-1, 1.22 g, 0.717 mmol) and Pd/C (10 wt. %, [BASF 4505 D/R E], 0.12 g, 1.127 mmol) in anhydrous THF (7 mL) was agitated under ˜0.1 bar H₂ atmosphere for 6 h at RT. The RM was then filtered and the collected solids washed with anhydrous THF (30 mL). The filtrate was concentrated under reduced pressure and the clear, colorless oil re-dissolved in anhydrous THF (5 mL). The solution was passed through an Agilent Stratospheres PL-Thiol MP cartridge (500 mg) and the cartridge washed with anhydrous THF (3×3 mL). The filtrate was concentrated under reduced pressure and the residue dissolved in Et₂O (5 mL). Enough heptane was added such that the solution became cloudy and emulsified. The mixture was cooled over dry ice for 30-40 min by which time hard wax-like solids had formed. The supernatant was then decanted and the remaining solids dissolved in DCM (10 mL). Concentration under reduced pressure provided the title compound Intermediate F4-Peg12-R (900 mg) as a clear, colorless oil.

### Example 35d: (S)-56-Carboxy-1,53,58-trioxo-1-(perfluorophenoxy)-57-undecyl-4,7,10,13,16,19,22,25,28,31,34,37,40,43,46,49-hexadecaoxa-52,57-diazaoctahexacontan-68-oic acid (Intermediate F4-Peg16)

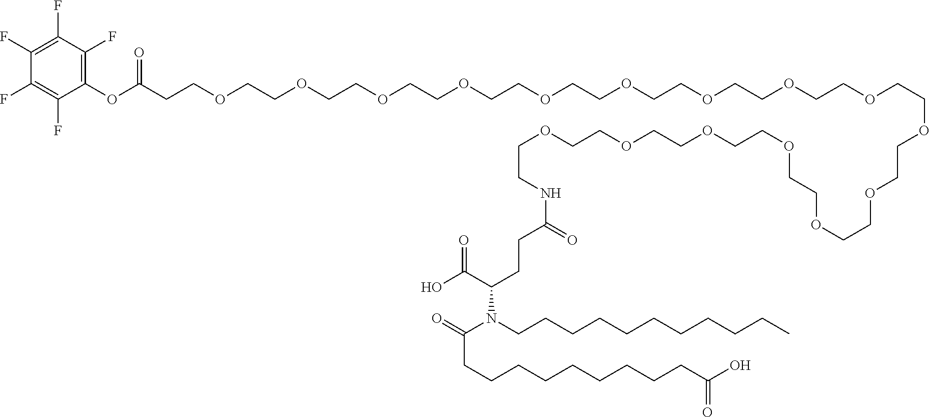 [SMILES: CCCCCCCCCCCN(C(=O)CCCCCCCCCC(=O)O)[C@@H](CCC(=O)NCCOCCOCCOCCOCCOCCOCCOCCOCCOCCOCCOCCOCCOCCOCCOCCOCCC(=O)Oc1c(F)c(F)c(F)c(F)c1F)C(=O)O]

### Step 1: 55,66-Dibenzyl 1-(perfluorophenyl) (S)-52,57-dioxo-56-undecyl-3,6,9,12,15,18,21,24,27,30,33,36,39,42,45,48-hexadecaoxa-51,56-diazahexahexacontane-1,55,66-tricarboxylate (Intermediate F4-PEG16-1)

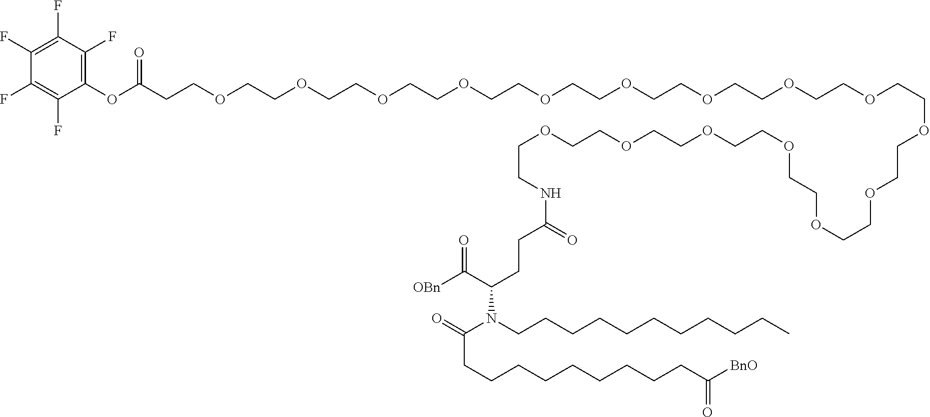 [SMILES: CCCCCCCCCCCN(C(=O)CCCCCCCCCC(=O)OCc1ccccc1)[C@@H](CCC(=O)NCCOCCOCCOCCOCCOCCOCCOCCOCCOCCOCCOCCOCCOCCOCCOCCOCCC(=O)Oc1c(F)c(F)c(F)c(F)c1F)C(=O)OCc1ccccc1]

This compound was prepared, using the appropriate starting materials, according to the procedures described for Intermediate F4-Peg12-S in Example 31d herein above with a modified purification. The crude RM was triturated sequentially with heptane (2×) and heptane/Et₂O (1/1). Each trituration involved ultrasonication of the oily residue (approx. 3 min) and then leaving the cloudy, heterogeneous mixture to stand in dry ice for 10 min. This allowed the target product to form into a white wax-like substance on the bottom (and walls) of the glass. The slightly cloudy organic solvent was then decanted. The solid was dried under reduced pressure providing crude Intermediate F4-Peg16-1 as a colorless oil. LCMS Method T11: Rt=1.42 min., MS m/z [M+2H]²⁺=811.9.

### Step 2: (S)-56-Carboxy-1,53,58-trioxo-1-(perfluorophenoxy)-57-undecyl-4,7,10,13,16,19,22,25,28,31,34,37,40,43,46,49-hexadecaoxa-52,57-diazaoctahexacontan-68-oic acid (Intermediate F4-Peg16)

In a 50 mL round-bottom flask equipped with a stir bar, 55,66-dibenzyl 1-(perfluorophenyl) (S)-52,57-dioxo-56-undecyl-3,6,9,12,15,18,21,24,27,30,33,36,39,42,45,48-hexadecaoxa-51,56-diazahexahexacontane-1,55,66-tricarboxylate (Intermediate F4-PEG16-1, 2 g, 1.23 mmol) was dissolved in EtOAc (10 mL). Pd(OH)₂/C (20 wt. %, wet, Degussa-type, 0.260 g, 0.370 mmol) was added, the flask sealed with a septa, and vacuum pulled on it through a needle for ˜1 min. The vacuum was then replaced by H₂ gas through a needle attached to a H₂-balloon, and the mixture stirred at RT for 4 h. Celite® was added to the RM, which was filtered through packed Celite® to trap unwanted solids. These solids were washed with MeOH (100 mL). The combined filtrate and MeOH wash were concentrated under reduced pressure to give the crude title compound Intermediate F4-Peg16 (1.8 g) as a black oil. LCMS Method T7: Rt=1.36 min/2.0 min; MS m/z [M+2H]²⁺=722.0.

### Example 36d: (S)-80-Carboxy-1,77,82-trioxo-1-(perfluorophenoxy)-81-undecyl-4,7,10,13,16,19,22,25,28,31,34,37,40,43,46,49,52,55,58,61,64,67,70,73-tetracosaoxa-76,81-diazadononacontan-92-oic acid (Intermediate F4-Peg24)

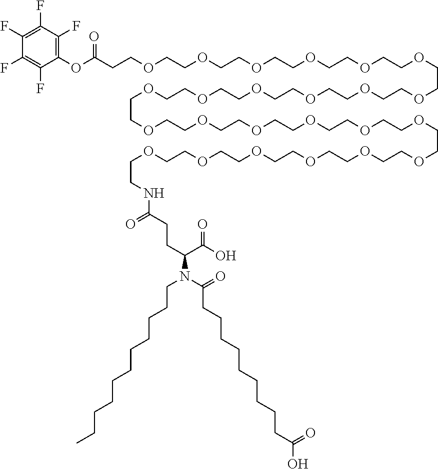 [SMILES: CCCCCCCCCCCN(C(=O)CCCCCCCCCC(=O)O)[C@@H](CCC(=O)NCCOCCOCCOCCOCCOCCOCCOCCOCCOCCOCCOCCOCCOCCOCCOCCOCCOCCOCCOCCOCCOCCOCCOCCOCCC(=O)Oc1c(F)c(F)c(F)c(F)c1F)C(=O)O]

### Step 1: 79,90-Dibenzyl 1-(perfluorophenyl) (S)-76,81-dioxo-80-undecyl-3,6,9,12,15,18,21,24,27,30,33,36,39,42,45,48,51,54,57,60,63,66,69,72-tetracosaoxa-75,80-diazanonacontane-1,79,90-tricarboxylate (Intermediate F4-Peg24-1)

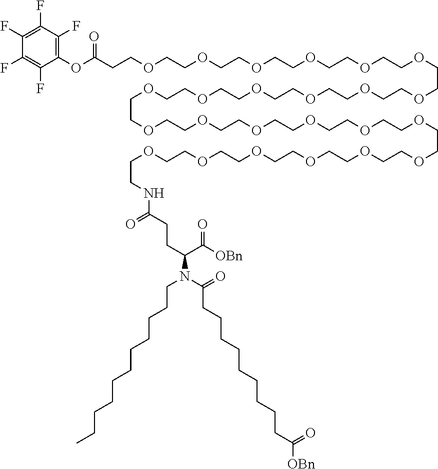 [SMILES: CCCCCCCCCCCN(C(=O)CCCCCCCCCC(=O)OCc1ccccc1)[C@@H](CCC(=O)NCCOCCOCCOCCOCCOCCOCCOCCOCCOCCOCCOCCOCCOCCOCCOCCOCCOCCOCCOCCOCCOCCOCCOCCOCCC(=O)Oc1c(F)c(F)c(F)c(F)c1F)C(=O)OCc1ccccc1]

To a solution of 1-benzyl 5-(perfluorophenyl) N-(11-(benzyloxy)-11-oxoundecanoyl)-N-undecyl-L-glutamate (Intermediate F4-S, 4.652 g, 5.46 mmol) and 1-amino-3,6,9,12,15,18,21,24,27,30,33,36,39,42,45,48,51,54,57,60,63,66,69,72-tetracosaoxapentaheptacontan-75-oic acid (6.13 g, 5.19 mmol) in anhydrous DCM (100 mL) was added DIPEA (4.77 mL, 27.3 mmol) and the RM stirred at RT for 1 h. Bis(pentafluorophenyl)carbonate (2.263 g, 5.57 mmol) was added and stirring continued for a further 1 h at RT. The RM was then concentrated under reduced pressure and the crude oily residue ultrasonically triturated with Et₂O (2×70 mL). After cooling the mixtures at 4° C., the supernatants were decanted and the remaining residue concentrated under reduced pressure to give the title compound Intermediate F4-Peg24-1 as a clear, almost colorless oil, which was carried on directly to the next step. (11.7 g) LCMS Method W6: Rt=1.70 min; MS m/z [M−H−Pfp]-=1806.2.

### Step 2: (S)-80-Carboxy-1,77,82-trioxo-1-(perfluorophenoxy)-81-undecyl-4,7,10,13,16,19,22,25,28,31,34,37,40,43,46,49,52,55,58,61,64,67,70,73-tetracosaoxa-76,81-diazadononacontan-92-oic acid (Intermediate F4-Peg24)

A suspension of 79,90-dibenzyl 1-(perfluorophenyl) (S)-76,81-dioxo-80-undecyl-3,6,9,12,15,18,21,24,27,30,33,36,39,42,45,48,51,54,57,60,63,66,69,72-tetracosaoxa-75,80-diazanonacontane-1,79,90-tricarboxylate (Intermediate F4-Peg24-1, 6.92 g, 3.16 mmol) and Pd/C (10 wt. %, [BASF 4505 D/R E], 0.699 g, 0.657 mmol) in anhydrous THF (60 mL) was agitated under 0.1 bar H₂ for 45 min at RT. The RM was filtered and the collected solids washed with anhydrous THF (20 mL). The combined filtrate and THF wash were passed through an Agilent Stratospheres PL-Thiol MP cartridge (5 g) and the cartridge washed with anhydrous THF (30 mL). The combined filtrate and THF wash were then concentrated under reduced pressure to give a clear, almost colorless oil. The oily product was ultrasonically triturated with Et₂O (2×40 mL) and, after cooling to 4° C., the supernatants were decanted. The remaining oily residue was concentrated under reduced pressure to give the title compound Intermediate F4-Peg24 (5.20 g) as a clear, golden-yellow oil. LCMS Method W6: Rt=1.40 min; MS m/z [M−H]⁻=1792.1.

### Intermediates N

### Example 4a: 2-(3-((2,4-Dioxotetrahydropyrimidin-1(2H)-yl)methyl)-2-oxopyridin-1(2H)-yl)acetaldehyde (Intermediate N1)

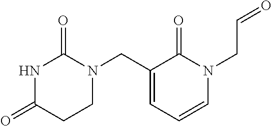 [SMILES: O=CCn1cccc(CN2CCC(=O)NC2=O)c1=O]

### Step 1: 3-(Aminomethyl)pyridin-2(1H)-one (Intermediate N1-1)

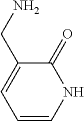

To a 1 L round bottom flask were added 2-oxo-1,2-dihydropyridine-3-carbonitrile (12 g, 100 mmol), Raney Ni (3 g), a solution of NH₃ (7 M) in MeOH (100 mL) and MeOH (150 mL). The RM was stirred under H₂ (1 atm) at RT for 48 h, filtered and the filtrate was concentrated, yielding the title compound Intermediate N1-1 as a yellow oil (13.5 g), which was used in the next step without further purification. LCMS Method T3: Rt=0.48 min; [M+H]⁺=125.

### Step 2: tert-Butyl ((2-oxo-1,2-dihydropyridin-3-yl)methyl)carbamate (Intermediate N1-2)

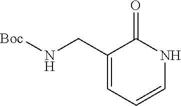 [SMILES: CC(C)(C)OC(=O)NCc1ccc[nH]c1=O]

To a 1 L round bottom flask were added 3-(aminomethyl)pyridin-2(1H)-one (Intermediate N1-1, 13.5 g, 100 mmol), DIEA (25.8 g, 200 mmol), MeOH (200 mL), DCM (300 mL) and di-tert-butyl dicarbonate (21.8 g, 100 mmol). The RM was stirred at RT for 16 h, concentrated and the residue was purified by chromatography on silica gel eluting with MeOH in DCM from 0 to 8% to afford the title compound Intermediate N1-2 as an oil (10.0 g). LCMS Method T4: Rt=1.61 min; [M+H]⁺=225.

### Step 3: tert-Butyl ((1-allyl-2-oxo-1,2-dihydropyridin-3-yl)methyl)carbamate (Intermediate N1-3)

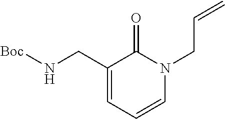 [SMILES: C=CCn1cccc(CNC(=O)OC(C)(C)C)c1=O]

To a 250 mL round bottom flask were added tert-butyl ((2-oxo-1,2-dihydropyridin-3-yl)methyl)carbamate (Intermediate N1-2, 10.0 g, 45 mmol), K₂CO₃ (12.4 g, 90 mmol), DMF (80 mL) and 3-bromoprop-1-ene (8.1 g, 67 mmol). The RM was stirred at RT for 16 h, filtered and the filtrate was poured into water (500 mL). The mixture was extracted with EtOAc (4×300 mL) and the combined organic phases were dried over Na₂SO₄, yielding the title compound Intermediate N1-3 as an oil (14.0 g). LCMS Method T4: Rt=1.78 min; [M+H]⁺=265.

### Step 4: 1-Allyl-3-(aminomethyl)pyridin-2(1H)-one (Intermediate N1-4)

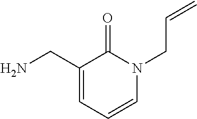

To a 1 L round bottom flask were added tert-butyl ((1-allyl-2-oxo-1,2-dihydropyridin-3-yl)methyl)carbamate (Intermediate N1-3, 14.0 g), DCM (300 mL), and a solution of HCl (4 M) in 1,4-dioxane (50 mL). The RM was stirred at RT for 16 h, the solvents were removed and the residue was purified by RP chromatography on a Biotage Agela C18 column (120 g, spherical 20-35 μm, 100 Å) eluting with ACN in aq. ammonium hydrogen carbonate (0.1%) from 5 to 40%, yielding the title compound Intermediate N1-4 as an oil (7.2 g). LCMS Method T4: Rt=1.14 min; [M+H]⁺=165.

### Step 5: 3-(((1-Allyl-2-oxo-1,2-dihydropyridin-3-yl)methyl)amino)propanoic acid (Intermediate N1-5)

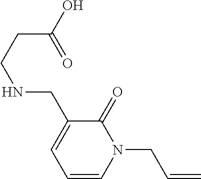 [SMILES: C=CCn1cccc(CNCCC(=O)O)c1=O]

To a 250 mL round bottom flask were added 1-allyl-3-(aminomethyl)pyridin-2(1H)-one (Intermediate N1-4, 3.28 g, 20 mmol), acrylic acid (4.32 g, 60 mmol) and toluene (100 mL). The RM was stirred at 100° C. for 18 h and concentrated to afford the crude title compound Intermediate N1-5, which was used in the next step without further purification. LCMS Method T5: Rt=0.34 min; [M+H]⁺=237.

### Step 6: 1-((1-Allyl-2-oxo-1,2-dihydropyridin-3-yl)methyl)dihydropyrimidine-2,4(1H,3H)-dione (Intermediate N1-6)

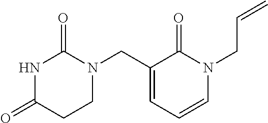 [SMILES: C=CCn1cccc(CN2CCC(=O)NC2=O)c1=O]

To a 250 mL round bottom flask were added 3-(((1-allyl-2-oxo-1,2-dihydropyridin-3-yl)methyl)amino)propanoic acid (Intermediate N1-5, 8 g), urea (3.6 g, 60 mmol) and acetic acid (40 mL). The RM was stirred at 120° C. for 18 h, concentrated and the residue was purified by RP chromatography on a Biotage Agela C18 column (120 g, spherical 20-35 μm, 100 Å) eluting with ACN in aq. ammonium hydrogen carbonate (0.1%) from 5 to 50%, yielding the title compound Intermediate N1-6 as a solid (3.4 g). LCMS Method T4: Rt=1.40 min; [M+H]⁺=262.

### Step 7: 2-(3-((2,4-Dioxotetrahydropyrimidin-1(2H)-yl)methyl)-2-oxopyridin-1(2H)-yl)acetaldehyde (Intermediate N1)

To a 250 mL round bottom flask were added 1-((1-allyl-2-oxo-1,2-dihydropyridin-3-yl)methyl)dihydropyrimidine-2,4(1H,3H)-dione (Intermediate N1-6, 3.9 g, 15 mmol), THF (120 mL), and a solution of OsO₄ (4%) in water (8 mL). The RM was stirred under N₂ atmosphere at RT for 45 min. Solid NaIO₄ (9.6 g, 45 mmol) was added and the RM was stirred under N₂ atmosphere at RT for 16 h. The mixture was filtered, the solvents were removed and the residue was purified by RP chromatography on a Biotage Agela C18 column (120 g, spherical 20-35 μm, 100 Å) eluting with ACN in aq. ammonium hydrogen carbonate (0.1%) from 0 to 30%, yielding the title compound Intermediate N1 (3.6 g) as a solid. LCMS Method T6: Rt=0.42 min; [M+H]⁺=264.

### Alternative Preparation of Intermediate N1

### Step 7a: 1-((1-(2,3-Dihydroxypropyl)-2-oxo-1,2-dihydropyridin-3-yl)methyl)dihydropyrimidine-2,4(1H,3H)-dione (Intermediate N1-7a)

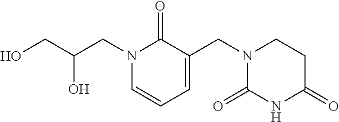 [SMILES: O=C1CCN(Cc2cccn(CC(O)CO)c2=O)C(=O)N1]

To a mixture of 1-((1-allyl-2-oxo-1,2-dihydropyridin-3-yl)methyl)dihydropyrimidine-2,4(1H,3H)-dione (Intermediate N1-6, 43 g, 164 mmol) in ACN (400 mL) and water (400 mL) was added KMnO₄ (31 g, 197 mmol) at 0° C. and the mixture was stirred at 25° C. for 12 h. The RM was filtered and the filter cake was washed with water (400 mL). The combined filtrate and water wash were concentrated under reduced pressure to give the crude title compound Intermediate N1-7a (45 g) as white solid. The crude product was used in the next step without purification.

### Step 7b: 2-(3-((2,4-Dioxotetrahydropyrimidin-1(2H)-yl)methyl)-2-oxopyridin-1(2H)-yl)acetaldehyde (Intermediate N1)

To a mixture of crude 1-((1-(2,3-dihydroxypropyl)-2-oxo-1,2-dihydropyridin-3-yl)methyl)dihydropyrimidine-2,4(1H,3H)-dione (Intermediate N1-7a) (45 g) in THF (400 mL) and water (400 mL) was added NaIO₄ (34 g, 159 mmol) at 0° C. and the mixture was stirred at 25° C. for 2 h. The RM was filtered and the filter cake was washed with water (400 mL). The combined filtrate and water wash were concentrated under reduced pressure to give the crude title compound. Combined with another batch of the same scale, the crude product was purified by preparative HPLC (eluting with ACN/water with formic acid as modifier). Product fractions were concentrated to remove ACN and the remaining aq. was dried by lyophilization providing the title compound Intermediate N1 (33 g) as a white solid.

### Example 5a: 4-(4-Chloro-7H-pyrrolo[2,3-d]pyrimidin-6-yl)benzaldehyde (Intermediate N2)

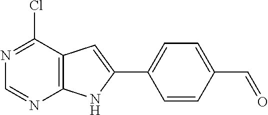 [SMILES: O=Cc1ccc(-c2cc3c(Cl)ncnc3[nH]2)cc1]

A mixture of 4-chloro-6-iodo-7H-pyrrolo[2,3-d]pyrimidine (50 g, 179 mmol), 4-(4,4,5,5-tetramethyl-1,3,2-dioxaborolan-2-yl)benzaldehyde (42 g, 180 mmol), PdCl₂(PPh₃)₂ (13 g, 17.9 mmol) and Cs₂CO₃ (69 g, 197 mmol) in dioxane (500 mL) and water (500 mL) was degassed and purged with N₂ three times. The RM was stirred at 100° C. for 4 h under N₂ atmosphere and then concentrated under reduced pressure to remove the dioxane. The resulting mixture was triturated with water (300 mL) and then filtered. The solids were triturated with EtOAc (2×300 mL) to give the title compound Intermediate N2 (41 g) as a yellow solid. ¹H NMR (400 MHz, DMSO-d₆) 5 [ppm] 13.35-13.09 (m, 1H), 10.06 (s, 1H), 8.72 (br s, 1H), 8.33-8.19 (m, 2H), 8.08-7.98 (m, 2H), 7.38-7.31 (m, 1H).

### Example 6a: 4-Chloro-6-(4-((4-(piperidin-4-yloxy)piperidin-1-yl)methyl)phenyl)-7H-pyrrolo[2,3-d]pyrimidine hydrochloride salt (Intermediate N3)

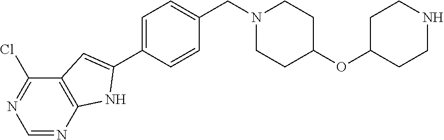 [SMILES: Clc1ncnc2[nH]c(-c3ccc(CN4CCC(OC5CCNCC5)CC4)cc3)cc12]

### Step 1: tert-Butyl 4-((1-(4-(4-chloro-7H-pyrrolo[2,3-d]pyrimidin-6-yl)benzyl)piperidin-4-yl)oxy)piperidine-1-carboxylate (Intermediate N3-1)

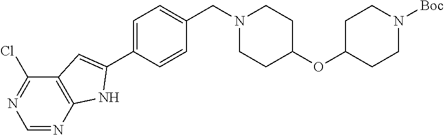 [SMILES: CC(C)(C)OC(=O)N1CCC(OC2CCN(Cc3ccc(-c4cc5c(Cl)ncnc5[nH]4)cc3)CC2)CC1]

A mixture of 4-(4-chloro-7H-pyrrolo[2,3-d]pyrimidin-6-yl)benzaldehyde (38 g, 147 mmol) and tert-butyl 4-(piperidin-4-yloxy)piperidine-1-carboxylate hydrochloride salt (52.0 g, 162 mmol), sodium acetate (24.2 g, 294 mmol, 2 eq), acetic acid (11.5 g, 191 mmol, 10.9 mL) in DMF (300 mL) was stirred at 25° C. for 0.5 h. NaBH₃CN (18.5 mg, 294 mmol) and MeOH (50 mL) were added to the RM and the mixture was stirred at 25° C. for 5 h. The RM was diluted with water (200 mL) and extracted with DCM (3×200 mL). The combined organic layers were washed with brine (200 mL), dried over Na₂SO₄ and concentrated under reduced pressure. The resulting residue was purified by column chromatography over silica (DCM/MeOH=1/0 to 10/1) providing the title compound Intermediate N3-1 (32.0 g) as a grey solid. ¹H NMR (400 MHz, DMSO-d₆) δ [ppm]13.26-12.88 (m, 1H), 8.69-8.53 (m, 1H), 8.16-7.94 (m, 2H), 7.45 (br s, 2H), 7.22-7.07 (m, 1H), 5.75 (s, 1H), 3.79-3.48 (m, 5H), 2.99 (br s, 3H), 2.29-2.03 (m, 1H), 1.94-1.68 (m, 5H), 1.59-1.42 (m, 2H), 1.38 (s, 9H), 1.29 (br d, J=8.8 Hz, 2H), 1.22 (br s, 1H).

### Step 2: 4-Chloro-6-(4-((4-(piperidin-4-yloxy)piperidin-1-yl)methyl)phenyl)-7H-pyrrolo[2,3-d]pyrimidine hydrochoride salt (Intermediate N3)

A solution of tert-butyl 4-((1-(4-(4-chloro-7H-pyrrolo[2,3-d]pyrimidin-6-yl)benzyl)piperidin-4-yl)oxy)piperidine-1-carboxylate (Intermediate N3-1, 58 g, 110 mmol) in DCM (600 mL) was added to HCl in dioxane (4M, 400 mL) at 0° C. The RM was stirred at 25° C. for 2 hand then concentrated to give the crude residue. The residue was suspended in MTBE (500 mL) and then filtered. The solid was dried to give the title compound Intermediate N3, (50 g) as a yellow solid. LCMS: MS m/z [M+H]⁺=426.3.

### Example 7a: 1-((2-Oxo-1-(2-(4-(piperidin-4-yloxy)piperidin-1-yl)ethyl)-1,2-dihydropyridin-3-yl)methyl)dihydropyrimidine-2,4(1H,3H)-dione (Intermediate N4)

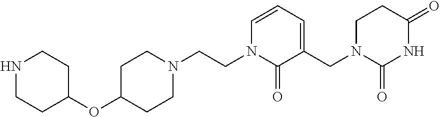 [SMILES: O=C1CCN(Cc2cccn(CCN3CCC(OC4CCNCC4)CC3)c2=O)C(=O)N1]

### Step 1: tert-Butyl 4-((1-(2-(3-((2,4-dioxotetrahydropyrimidin-1(2H)-yl)methyl)-2-oxopyridin-1(2H)-yl)ethyl)piperidin-4-yl)oxy)piperidine-1-carboxylate (Intermediate N4-1)

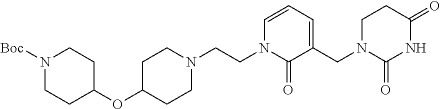 [SMILES: CC(C)(C)OC(=O)N1CCC(OC2CCN(CCn3cccc(CN4CCC(=O)NC4=O)c3=O)CC2)CC1]

To a 250 mL round bottom flask was added 2-(3-((2,4-dioxotetrahydropyrimidin-1(2H)-yl)methyl)-2-oxopyridin-1(2H)-yl)acetaldehyde (Intermediate N1, 3.6 g, 13.6 mmol), tert-butyl 4-(piperidin-4-yloxy)piperidine-1-carboxylate (3.86 g, 13.6 mmol), a solution of ZnCl₂ in THF (1M, 20.4 mL, 20.4 mmol) and DMSO (40 mL). The RM was stirred at RT for 2 h. Solid NaBH₃CN (2.57 g, 40.8 mmol) and MeOH (8 mL) were then added. The resulting mixture was stirred at RT for 16 h, concentrated under reduced pressure, and then purified by RP chromatography on a Biotage Agela C18 column (120 g, spherical 20-35 μm, 100 Å) eluting with ACN in aq. ammonium hydrogen carbonate (0.1%) from 5 to 60%, yielding the title compound Intermediate N4-1 (2.8 g). as a solid. LCMS Method T3: Rt=1.81 min; MS m/z [M+H]⁺=532.

### Step 2: 1-((2-Oxo-1-(2-(4-(piperidin-4-yloxy)piperidin-1-yl)ethyl)-1,2-dihydropyridin-3-yl)methyl)dihydropyrimidine-2,4(1H,3H)-dione (Intermediate N4)

To a 250 mL round bottom flask was added tert-butyl 4-(1-(2-(3-((2,4-dioxotetrahydropyrimidin-1(2H)-yl)methyl)-2-oxopyridin-1(2H)-yl)ethyl)piperidin-4-yloxy)piperidine-1-carboxylate (Intermediate N4-1, 2.8 g, 5.2 mmol), DCM (30 mL) and a solution of HCl in 1,4-dioxane (4M, 10 mL). The RM was stirred at RT for 6 h. The mixture was then concentrated under reduced pressure and purified by RP chromatography on a Biotage Agela C18 column (120 g, spherical 20-35 μm, 100 Å) eluting with ACN in aq. ammonium hydrogen carbonate (0.1%) from 0 to 50%, yielding the title compound Intermediate N4 (1.8 g) as a solid. LCMS Method T4: Rt=1.36 min; [M+H]⁺=432.

### Example 8a: 1-((1-(2-(4-((1-(4-(4-Chloro-7H-pyrrolo[2,3-d]pyrimidin-6-yl)benzyl)piperidin-4-yl)oxy)piperidin-1-yl)ethyl)-2-oxo-1,2-dihydropyridin-3-yl)methyl)dihydropyrimidine-2,4(1H,3H)-dione (Intermediate N5)

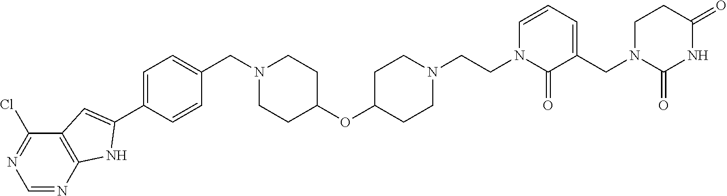 [SMILES: O=C1CCN(Cc2cccn(CCN3CCC(OC4CCN(Cc5ccc(-c6cc7c(Cl)ncnc7[nH]6)cc5)CC4)CC3)c2=O)C(=O)N1]

A mixture of 4-chloro-6-(4-((4-(piperidin-4-yloxy)piperidin-1-yl)methyl)phenyl)-7H-pyrrolo[2,3-d]pyrimidine hydrochloride salt (Intermediate N3, 28.0 g, 52.3 mmol) and 2-(3-((2,4-dioxotetrahydropyrimidin-1 (2H)-yl)methyl)-2-oxopyridin-1 (2H)-yl)acetaldehyde (Intermediate N1, 27.5 g, 104 mmol), sodium acetate (12.8 g, 156 mmol), acetic acid (15.7 g, 261 mmol, 14.9 mL), TEA (15.8 g, 156 mmol, 21.8 mL) in DMSO (150 mL) and DCM (250 mL) was stirred at 25° C. for 0.5 hr. NaBH(OAc)₃ (22.1 g, 104 mmol) was added to the mixture in portions and the mixture was stirred at 25° C. for 2 h. The pH of the mixture was adjusted to pH 7 with an aq. sat. NaHCO₃ solution. The mixture was then diluted with water (250 mL) and extracted with DCM (3×250 mL). The combined organic layers were washed with brine (2×250 mL), dried over Na₂SO₄, filtered and concentrated under reduced pressure. The resulting residue was purified by column chromatography (SiO₂, eluting with DCM/Methanol=1/0 to 10/1) to give the enriched title compound (21.0 g). Further purification of 10.0 g by RP HPLC (ACN/water with 0.1% TFA as modifier) provided the TFA salt of the title compound Intermediate N5 (8.80 g) as a yellow solid. LCMS: MS m/z [M+H]⁺=673.5.

### Example 9a: 2-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl (tert-butoxycarbonyl)glycinate (Intermediate N-A-P)

### Method 1

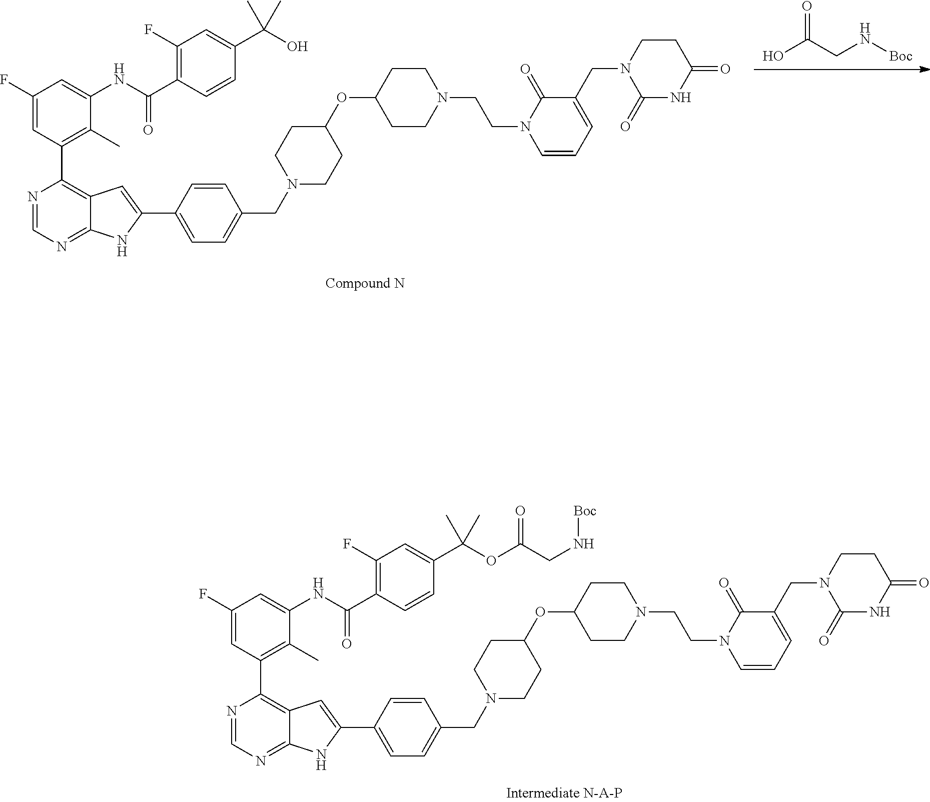 [SMILES: Cc1c(NC(=O)c2ccc(C(C)(C)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12 ; CC(C)(C)OC(=O)NCC(=O)O ; Cc1c(NC(=O)c2ccc(C(C)(C)OC(=O)CNC(=O)OC(C)(C)C)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

Two parallel reactions with Compound N (330 mg and 220 mg): To a mixture of Compound N (330 mg, 0.350 mmol), (tert-butoxycarbonyl)glycine (307 mg, 1.75 mmol), and DMAP (428 mg, 3.50 mmol) was added DCM (4.4 mL). HATU was then added (666 mg, 1.75 mmol) and stirring was continued under an atmosphere of N₂. The resulting solution was stirred for ˜48 h. The RM was diluted with DCM and washed with sat. aq. NaHCO₃ solution. The separated aq. layer was extracted with DCM and EtOAc and the combined organic layers were dried over Na₂SO₄, filtered, and concentrated under reduced pressure. The crude residue was pre-purified by column chromatography [SiO₂, eluting with DCM/MeOH=1/0 to 3/1]. The parallel reaction with Compound N (220 mg) was carried out using a similar protocol to the one described herein above, with stirring continuing for ˜16 h. Final purification of the combined material was achieved via preparative HPLC on a RP-HPLC C18 column eluting with ACN/water (0.1% formic acid as modifier) from 10 to 50% providing Intermediate N-A-P (102 mg) as the formate salt. ¹H NMR (600 MHz, DMSO-d₆) δ [ppm] 12.76 (d, J=2.0 Hz, 1H), 10.15 (s, 1H), 10.04 (d, J=2.1 Hz, 1H), 8.86 (s, 1H), 8.18 (s, 1H), 7.96-7.91 (m, 2H), 7.75 (t, J=7.7 Hz, 1H), 7.65 (dd, J=10.2, 2.8 Hz, 1H), 7.58 (dd, J=6.8, 2.0 Hz, 1H), 7.42-7.33 (m, 4H), 7.30-7.18 (m, 3H), 6.83 (d, J=2.0 Hz, 1H), 6.20 (t, J=6.8 Hz, 1H), 4.27 (s, 2H), 3.99 (t, J=6.5 Hz, 2H), 3.71-3.66 (m, 2H), 3.48 (s, 2H), 3.45-3.22 (m, 4H), 2.76-2.70 (m, 2H), 2.69-2.64 (m, 2H), 2.57 (t, J=6.8 Hz, 2H), 2.53-2.52 (m, 2H), 2.18 (s, 3H), 2.15-2.05 (m, 4H), 1.80-1.74 (m, 4H), 1.73 (s, 6H), 1.39 (s, 9H), 1.47-1.30 (m, 4H). LCMS Method T7: Rt=0.80 min; MS m/z [M−H]⁻=1097.8.

### Alternative Preparation of Intermediate N-A-P (Method 2)

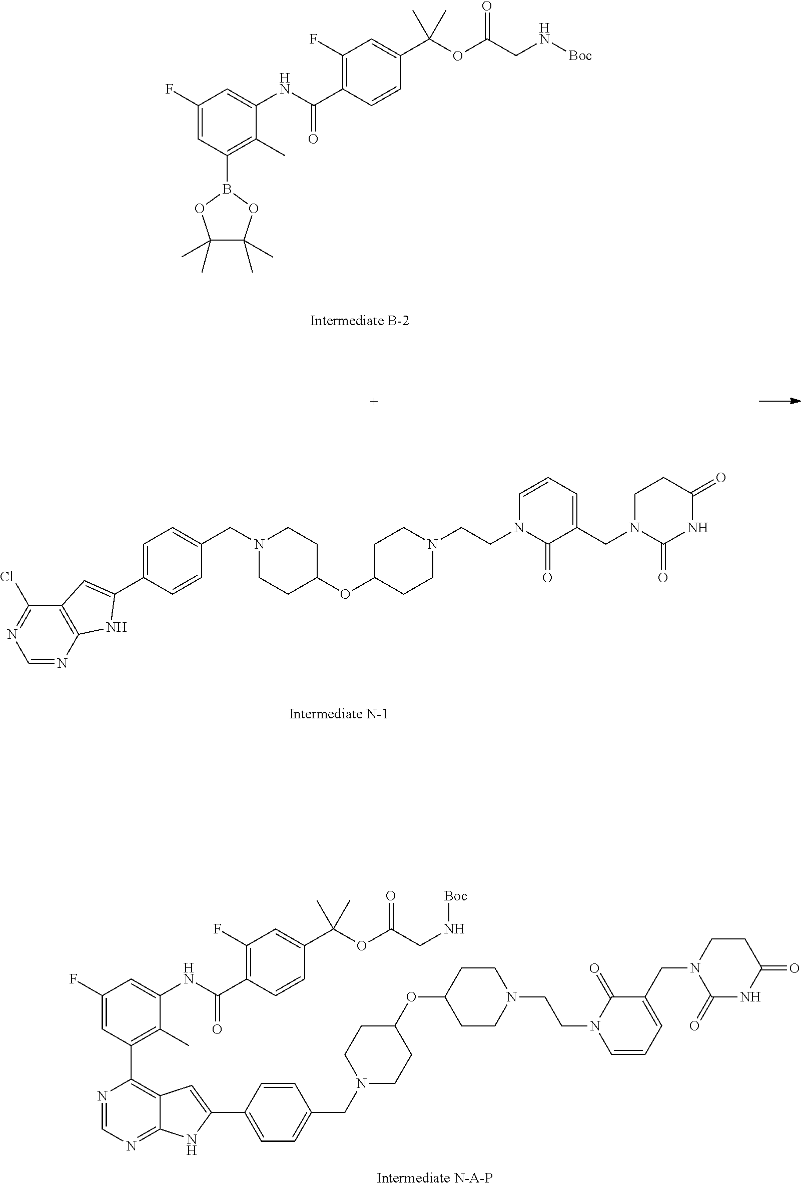 [SMILES: Cc1c(NC(=O)c2ccc(C(C)(C)OC(=O)CNC(=O)OC(C)(C)C)cc2F)cc(F)cc1B1OC(C)(C)C(C)(C)O1 ; O=C1CCN(Cc2cccn(CCN3CCC(OC4CCN(Cc5ccc(-c6cc7c(Cl)ncnc7[nH]6)cc5)CC4)CC3)c2=O)C(=O)N1 ; Cc1c(NC(=O)c2ccc(C(C)(C)OC(=O)CNC(=O)OC(C)(C)C)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

A mixture of 1-((1-(2-(4-((1-(4-(4-chloro-7H-pyrrolo[2,3-d]pyrimidin-6-yl)benzyl)piperidin-4-yl)oxy)piperidin-1-yl)ethyl)-2-oxo-1,2-dihydropyridin-3-yl)methyl)dihydropyrimidine-2,4(1H,3H)-dione (Intermediate N1, 1500 mg, 2.23 mmol), 2-(3-fluoro-4-((5-fluoro-2-methyl-3-(4,4,5,5-tetramethyl-1,3,2-dioxaborolan-2-yl)phenyl)carbamoyl)phenyl)propan-2-yl (tert-butoxycarbonyl)glycinate (Intermediate B2, 1403 mg), cataCXium® A Pd G3 (162 mg, 0.223 mmol) and an aq. solution of potassium phosphate (1M, 4.5 mL) in n-butanol (15 mL) was purged with N₂ for 10 min. The RM was heated at 100° C. for 3 h, allowed to cool to RT, and then concentrated under reduced pressure. The resulting residue was dissolved in DMSO (˜10 mL) and purified in 4 portions on an Interchim Puriflash HQ C18 column (120 g, spherical silica, 15 μm) eluting with 10 to 50% ACN/water (0.1% formic acid as modifier). Fractions were combined and lyophilized providing the title compound Intermediate N-A-P (1724 mg) as a white solid. LCMS Method T8: Rt=1.58 min; MS m/z [M+H]⁺=1100.9.

### Example 10a: 2-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl bis(tert-butoxycarbonyl)glycinate (Intermediate N-A-P2)

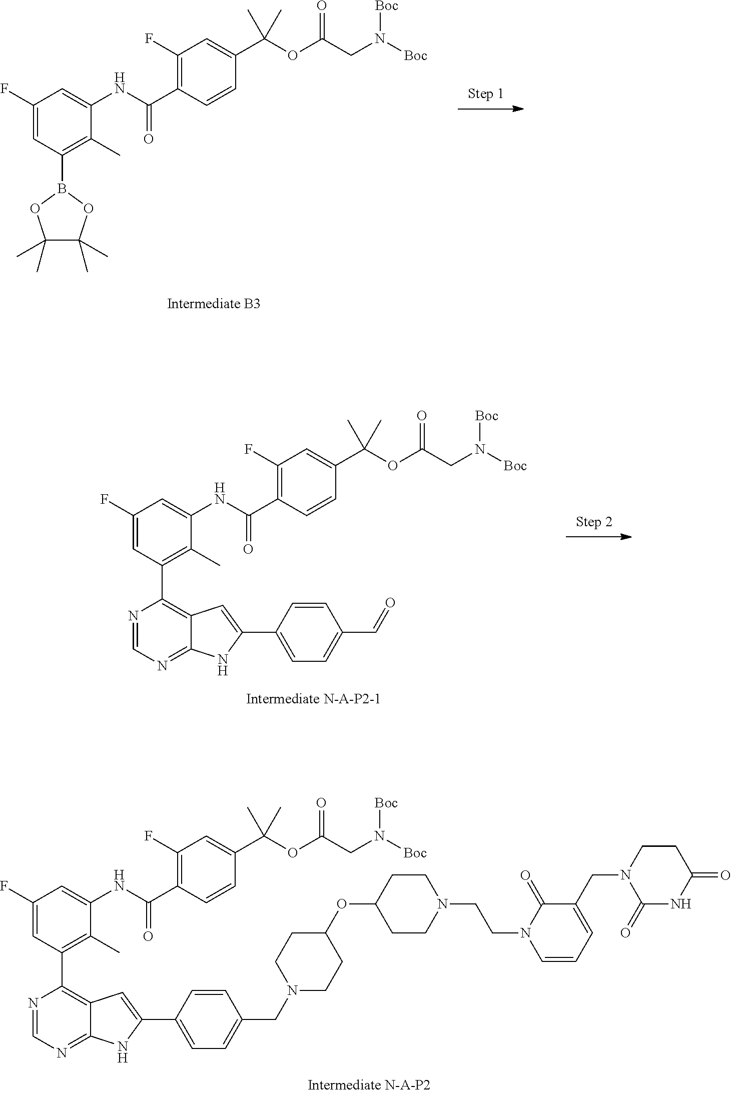 [SMILES: Cc1c(NC(=O)c2ccc(C(C)(C)OC(=O)CN(C(=O)OC(C)(C)C)C(=O)OC(C)(C)C)cc2F)cc(F)cc1B1OC(C)(C)C(C)(C)O1 ; Cc1c(NC(=O)c2ccc(C(C)(C)OC(=O)CN(C(=O)OC(C)(C)C)C(=O)OC(C)(C)C)cc2F)cc(F)cc1-c1ncnc2[nH]c(-c3ccc(C=O)cc3)cc12 ; Cc1c(NC(=O)c2ccc(C(C)(C)OC(=O)CN(C(=O)OC(C)(C)C)C(=O)OC(C)(C)C)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

### Step 1: 2-(3-Fluoro-4-((5-fluoro-3-(6-(4-formylphenyl)-7H-pyrrolo[2,3-d]pyrimidin-4-yl)-2-methylphenyl)carbamoyl)phenyl)propan-2-yl bis(tert-butoxycarbonyl)glycinate (Intermediate N-A-P2-1)

A mixture of 4-(4-chloro-7H-pyrrolo[2,3-d]pyrimidin-6-yl)benzaldehyde (Intermediate N2, 20 g, 77.62 mmol), 2-(3-fluoro-4-((5-fluoro-2-methyl-3-(4,4,5,5-tetramethyl-1,3,2-dioxaborolan-2-yl)phenyl)carbamoyl)phenyl)propan-2-yl bis(tert-butoxycarbonyl)glycinate (Intermediate B3, 52.9 g, 76.83 mmol), Na₂CO₃ (7.6 g, 71.70 mmol), 2-MeTHF (150 mL) and water (50 mL) was purged with N₂ three times, and then Pd(PPh₃)₂Cl₂ (2.5 g, 3.56 mmol) was added under N₂ atmosphere. The mixture was stirred at ˜75° C. for 16 h. The temperature was adjusted to ˜20° C., and the mixture was diluted with THF (150 mL). Sodium acetyl-L-cysteinate (15%, 150 g) was then added and the mixture was stirred at −60° C. for over 5 h. A NaCl solution (10%, 150 g) was added and the mixture was stirred at ˜20° C. for 5 min. After phase separation, water (150 g) was added to the organic layer, and the mixture was stirred for 5 min. The separated organic layer was filtered through MCC and the cake was washed with 2-MeTHF (2×30 mL). Smopex-234pp (8 g) was added to the organic layer and the mixture was stirred at ˜60° C. for over 16 h. After filtration through MCC, the filter cake was washed with 2-MeTHF (2×30 mL). The combined filtrate and the 2-MeTHF washes were concentrated under reduced pressure (50-100 mbar, ˜40° C. water bath) to dryness and 2-MeTHF (160 mL) was then added. The mixture was stirred at ˜40° C. and ACN (640 mL) was added dropwise over 1 h. The mixture was concentrated under reduced pressure (50-100 mbar, ˜40° C. water bath) to a volume of about 400 mL, and then stirred at ˜40° C. while ACN (600 mL) was added dropwise over 1 h, and stirring was continued for additional 3 h. The mixture was allowed to cool down to 20° C. over 5 h and stirred at ˜20° C. for over 5 h. After filtration, the cake was washed with ACN (2×50 mL). The wet cake was dried under vacuum at ˜40° C. for at least 5 h providing the title compound Intermediate N-A-P2-1 (43.3 g) as a yellow solid, which was used without further purification. LCMS Method C1: HRMS m/z [M+H]+: 783.9734.

### Step 2: 2-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl bis(tert-butoxycarbonyl)glycinate (Intermediate N-A-P2)

A mixture of 2-(3-fluoro-4-((5-fluoro-3-(6-(4-formylphenyl)-7H-pyrrolo[2,3-d]pyrimidin-4-yl)-2-methylphenyl)carbamoyl)phenyl)propan-2-yl bis(tert-butoxycarbonyl)glycinate (Intermediate N-A-P2-1, 15 g, 19.14 mmol), 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione dihydrochloride (Intermediate N4, 14.0 g), DCM (225 mL) and TEA (7.75 g, 76.59 mmol) was stirred at ˜35° C. for 3 h. NaBH(OAc)₃ (16.2 g, 76.44 mmol) was added in five portions over 30 min while keeping the mixture at ˜35° C. The RM was stirred at ˜35° C. for 8 h. An aq. solution of NaHCO₃ (5%, 120 mL) and MeOH (90 mL) were added and stirring was continued for 30 min. After phase separation, an aq. solution of NaHCO₃ (5%, 120 mL) and MeOH (68 mL) were added to the organic layer and stirring was continued for 30 min. After phase separation, an aq. solution of NaCl (10%, 120 mL) and MeOH (68 mL) were added to the organic layer and stirring was continued for 30 min. After phase separation, MgSO₄ (15 g) was then added to the organic layer and the mixture was stirred for 1 h. The mixture was filtered through MCC and the wet cake was washed with DCM (2×30 mL). The combined filtrate and DCM washes were concentrated under reduced pressure (50-100 mbar, 40° C. water bath). Most of the solvent was removed to give the crude product. Further purification via L-tartaric salt formation: MeOH (135 mL) was added to the residue and the resulting solution was concentrated under reduced pressure (50-100 mbar, 40° C. water bath). After most of the solvent was removed additional MeOH (135 mL) was added. The solution was concentrated under reduced pressure (50-100 mbar, 40° C. water bath). MeOH (108 mL) was added again to the above residue to form a MeOH solution.

A mixture of L-tartaric acid (4.5 g, 1.6 eq) and IPA (225 mL) was stirred at ˜35° C. for 1 h and the above MeOH solution was then added dropwise. The resulting mixture was stirred at ˜35° C. for 1 h. The mixture was cooled to ˜25° C. and stirring was continued for 1 h. The mixture was filtered and the wet cake was washed with IPA (60 mL). The wet cake was transferred to a container and DCM (225 mL) and MeOH (66 mL) were added. To the resulting solution was added an aq. solution of NaHCO₃ (5%, 120 mL) and the mixture was stirred for 30 min. After phase separation, an aq. solution of NaHCO₃ (5%, 120 mL) and MeOH (44 mL) were added to the organic layer and stirring was continued for 30 min. After phase separation, an aq. solution of NaCl (10%, 120 mL) and MeOH (44 mL) were added to the organic layer. After phase separation, MgSO₄ (15 g) was then added to the organic layer and the mixture was stirred for 30 min and filtered. The filtrate was concentrated under reduced pressure (50-100 mbar, 40° C. water bath) providing the title compound Intermediate N-A-P2 (22.5 g) as a foamy solid which was used without further purification. LCMS Method C1: HRMS m/z [M+H]+: 1199.5745.

### Example 11a: 2-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl glycinate (Intermediate N-A)

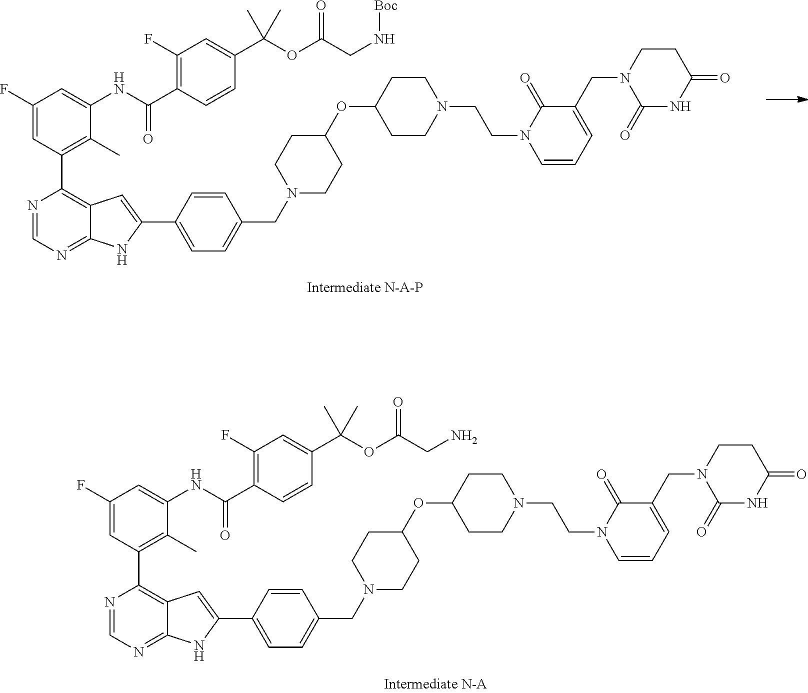 [SMILES: Cc1c(NC(=O)c2ccc(C(C)(C)OC(=O)CNC(=O)OC(C)(C)C)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12 ; Cc1c(NC(=O)c2ccc(C(C)(C)OC(=O)CN)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

To a solution of 2-(4-((3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl (tert-butoxycarbonyl)glycinate (Intermediate N-A-P, 2.94 g, 2.040 mmol) in CH₂Cl₂ (60 mL) at 0° C. was added Et₃Si (3.26 mL, 20.40 mmol), followed by TFA (15.72 mL, 204 mmol). The resulting mixture was stirred at 0° C. for 1.5 h. To the RM was then added ice-cold toluene (120 mL) and the resulting mixture was concentrated under reduced pressure at RT. The resulting residue was again treated with ice-cold toluene and concentrated under reduced pressure. The semi-solid residue was then treated with Et₂O (100 mL) and stirred at RT for 0.5 h. The liquid was decanted off of the solid, the solid was treated with Et₂O (100 mL) and stirred for 15 min. The resulting solid that formed was filtered under N₂ atmosphere and washed with Et₂O. Drying under high vacuum provided the TFA salt of the title compound Intermediate N-A (3.0 g) as yellow solid. LCMS Method T9: Rt=0.94 min; MS m/z [M+H]⁺=1000.

### Alternative Preparation of Intermediate N-A

A mixture of 2-(4-((3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl bis(tert-butoxycarbonyl)glycinate (Intermediate N-A-P2, 20 g, 16.68 mmol) and tert-butyl acetate (15.5 g, 133.4 mmol) in DCM (500 mL) was stirred at ˜20° C. and a solution of iodotrimethylsilane in DCM (1M, 100 mL) was then added dropwise over 1 h. The resulting mixture was stirred at 20° C. for 1 h. TEA (10.1 g, 100 mmol) was added to the mixture dropwise over 10 min. The RM as diluted with MeOH (200 mL) and an aq. solution of NaHCO₃ (5%, 400 mL) and stirring was continued at ˜20° C. for 30 min. After phase separation, the organic phase was filtered through MCC and the cake was washed with DCM (2×30 mL). To the combined filtrate and DCM washes were added MeOH (200 mL) and an aq. solution of NaCl (5%, 400 mL). After stirring at ˜20° C. for 10 min, the separated organic layer was concentrated under reduced pressure (50-100 mbar, ˜40° C. water bath) to dryness providing the crude Intermediate N-A (14 g) as a yellow solid which was used without further purification. LCMS Method C1: HRMS m/z [M+H]+: 999.4695.

### Example 12a: 13-(Benzyloxy)-2-((benzyloxy)carbonyl)-13-oxo-2-undecyltridecanoic acid (Intermediate rac-F1)

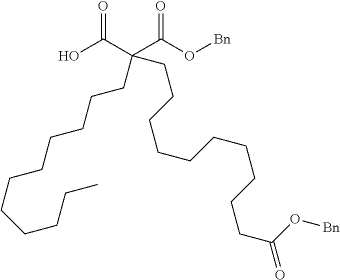 [SMILES: CCCCCCCCCCCC(CCCCCCCCCCC(=O)OCc1ccccc1)(C(=O)O)C(=O)OCc1ccccc1]

### Step 1: Benzyl 11-bromoundecanoate (Intermediate rac-F1-1)

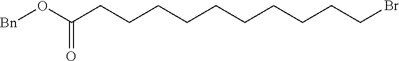 [SMILES: O=C(CCCCCCCCCCBr)OCc1ccccc1]

To a mixture of 11-bromoundecanoic acid (4.60 kg, 17.3 mol) in DCM (26.5 kg) was added EDCl (3.8 kg, 20.2 mol) in portions at 0° C. along with DMAP (98 g, 0.8 mol, 0.05 equiv). BnOH (1.70 kg, 15.7 mol) was then added dropwise. After stirring at 20° C. for 4 h, water (70.0 kg) was added slowly. The mixture was then concentrated under vacuum. Heptane (23.2 kg) and 19% NaCl solution (17 kg) were added for phase separation. The organic phase was washed 2× under basic condition (5% Na₂CO₃, 25.0 kg; 19% NaCl solution, 25.0 kg), 1× under acidic condition (5.2% HCl aq. solution, 25.0 kg; 19% NaCl solution, 25.0 kg), 1× with water (5.0 kg), and 1× with brine (5.0 kg). The organic layer was then concentrated under vacuum at 50° C. to provide the title compound Intermediate rac-F1-1, which was used in the next step without purification. ¹H NMR (400 MHz, Chloroform-d) δ [ppm] 1.18-1.36 (m, 10H), 1.37-1.47 (m, 2H), 1.64 (quin, J=7.33 Hz, 2H), 1.85 (dt, J=14.56, 7.06 Hz, 2H), 2.35 (t, J=7.58 Hz, 2H), 3.40 (t, J=6.88 Hz, 2H), 5.11 (s, 2H), 7.28-7.45 (m, 5H).

### Step 2: 1,11-Dibenzyl 11-(tert-butyl) docosane-1,11,11-tricarboxylate (Intermediate rac-F1-2)

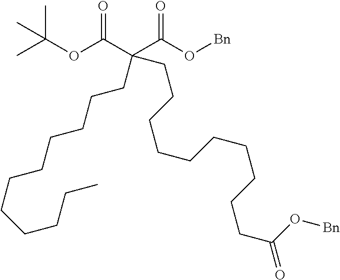 [SMILES: CCCCCCCCCCCC(CCCCCCCCCCC(=O)OCc1ccccc1)(C(=O)OCc1ccccc1)C(=O)OC(C)(C)C]

To a solution of benzyl tert-butyl malonate (3.0 kg, 12.0 mol) in NMP (30 L) was added 1-iodoundecane (3.55 kg, 12.58 mol) and Cs₂CO₃ (11.76 kg, 36.09 mol) at 20° C. The mixture was stirred at 20° C. for 6 h. To the mixture was then added benzyl 11-bromoundecanoate (Intermediate rac-F1-1, 5.53 kg, 15.6 mol) and the RM was heated to 80° C. and stirred for 12 h. The mixture was then cooled to 20° C. and a mixture of water (30 kg) and heptane (10 kg) was added. After stirring for 30 min, the organic layer was separated and washed 3× with a mixture of brine (5 kg) and MeOH (4 kg). The organic layer was dried over Na₂SO₄, filtered, and concentrated in vacuo. The crude product was purified by column chromatography eluting with heptane/EtOAc=1/0 to 100/1 to provide the title compound Intermediate rac-F1-2 (4.4 kg). ¹H NMR (400 MHz, Chloroform-d) δ [ppm] 0.84-0.94 (m, 3H), 1.12 (m, J=6.60 Hz, 4H), 1.19-1.33 (m, 28H), 1.35 (s, 9H), 1.66 (quin, J=7.40 Hz, 2H), 1.85 (t, J=8.44 Hz, 4H), 2.37 (t, J=7.52 Hz, 2H), 5.14 (s, 2H), 5.16 (s, 2H), 7.30-7.42 (m, 10H).

### Step 3: 13-(Benzyloxy)-2-((benzyloxy)carbonyl)-13-oxo-2-undecyltridecanoic acid (Intermediate rac-F1)

To a solution of 1,11-dibenzyl 11-(tert-butyl) docosane-1,11,11-tricarboxylate (Intermediate rac-F1-2, 6.0 kg, 8.8 mol) in heptane (21 L) was added TFA (10.0 kg, 88.4 mol) dropwise at 20±5° C. After stirring at 20±5° C. for 8 h, most of the TFA was removed under reduced pressure. The residue was re-dissolved in heptane (42 L) and washed with brine (42 L×3). After separation of the phases, the organic phase was concentrated to provide the crude product as a yellow oil. The crude product was purified by column chromatography eluting with heptane to heptane:EtOAc=10/1 to provide title compound Intermediate rac-F1 (4.6 kg). ¹H NMR (400 MHz, Chloroform-d) δ [ppm] 0.87-0.94 (m, 3H), 0.94-1.05 (m, 2H), 1.19 (br s, 14H), 1.23-1.37 (m, 16H), 1.65 (quin, J=7.40 Hz, 2H), 1.78-1.91 (m, 2H), 1.93-2.05 (m, 2H), 2.37 (t, J=7.52 Hz, 2H), 5.14 (s, 2H), 5.27 (s, 2H), 7.31-7.44 (m, 10H). LCMS Method C6: Rt=16.44 min/18.0 min, MS m/z [M+Na]+645.4.

### Example 13a: (R)-13-(Benzyloxy)-2-((benzyloxy)carbonyl)-13-oxo-2-undecyltridecanoic acid and (S)-13-(benzyloxy)-2-((benzyloxy)carbonyl)-13-oxo-2-undecyltridecanoic acid (Intermediate ent-F1-Peak1 and Intermediate ent-F1-Peak2)

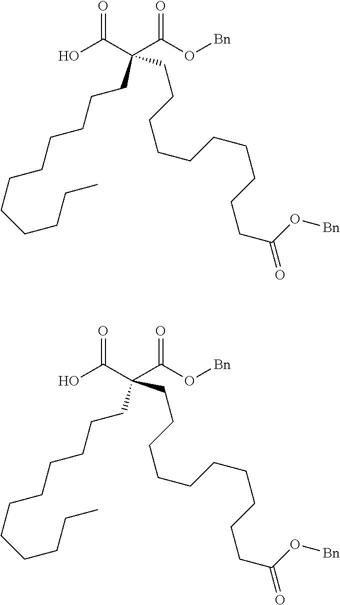 [SMILES: CCCCCCCCCCC[C@@](CCCCCCCCCCC(=O)OCc1ccccc1)(C(=O)O)C(=O)OCc1ccccc1 ; CCCCCCCCCCC[C@](CCCCCCCCCCC(=O)OCc1ccccc1)(C(=O)O)C(=O)OCc1ccccc1]

For chiral separation, 13-(benzyloxy)-2-((benzyloxy)carbonyl)-13-oxo-2-undecyltridecanoic acid (Intermediate rac-F1, 510 g) was dissolved in EtOH (25.5 L) and injected in 15 mL portions onto the following instrument and column: Instrument: Thar 350 preparative SFC (SFC-18); Column: ChiralPak AD, 300×50 mm I.D., 10 μm; Mobile phase: A for CO₂ and B for EtOH; Gradient: B 40%; Flow rate: 200 mL/min; Back pressure: 100 bar; Column temperature: 38° C.; Wavelength: 210 nm; Cycle time: ˜3.7 min. Product fractions were concentrated under reduced pressure at 40° C. providing:

### Enantiomer A (Intermediate Ent-F1-Peak1)

Eluting from the column at 4.06 min, ee 99.3% (Method C5), 222.88 g.
LCMS Method C2: Rt=3.77 min/5.0 min; MS m/z [M+H]⁺=623.5.

### Enantiomer B (Intermediate Ent-F1-Peak2)

Eluting from the column at 4.39 min, ee 98.8% (Method C5), 215.88 g.
LCMS Method C2: Rt=3.75 min/5.0 min; MS m/z [M+H]⁺=623.5.

### Example 14a: 17-(Benzyloxy)-2-(15-(benzyloxy)-15-oxopentadecyl)-2-((benzyloxy)carbonyl)-17-oxoheptadecanoic acid (Intermediate F2)

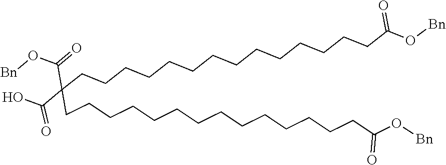 [SMILES: O=C(CCCCCCCCCCCCCCC(CCCCCCCCCCCCCCC(=O)OCc1ccccc1)(C(=O)O)C(=O)OCc1ccccc1)OCc1ccccc1]

### Step 1: Methyl 15-hydroxypentadecanoate (Intermediate F2-1)

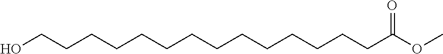 [SMILES: COC(=O)CCCCCCCCCCCCCCO]

To a solution of oxacyclohexadecan-2-one (137 g, 569 mmol) in MeOH (1 L) was added NaOMe (5M, 42.1 mL) at 18° C. The resulting solution was stirred at 18° C. for 21 h. Once TLC indicated a complete consumption of the starting material (PE/Et₂O=5/1), the RM was quenched with aq. HCl (1M, 400 mL). MeOH was removed under reduced pressure, which left behind a suspension of solids. The solids were filtered providing the crude title compound Intermediate F2-1 (134 g) which was taken onto the next step without further purification. ¹H NMR (400 MHz, Chloroform-d) δ [ppm] 3.67 (s, 3H), 3.64 (t, J=6.8 Hz, 2H), 2.31 (t, J=7.6 Hz, 2H), 1.52-1.67 (m, 4H), 1.21-1.39 (m, 20H).

### Step 2: Methyl 15-bromopentadecanoate (Intermediate F2-2)

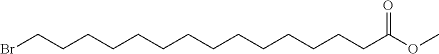 [SMILES: COC(=O)CCCCCCCCCCCCCCBr]

Methyl 15-hydroxypentadecanoate (Intermediate F2-1, 134 g, 491 mmol) was added to carbon tetrabromide (309 g, 934 mmol) and dissolved in DCM (1.5 L). The resulting mixture was cooled to 0° C. and PPh₃ (245 g, 934 mmol) was added in portions to the mixture. The RM was allowed to warm to 18° C. and then stirred for 16 h. Once TLC indicated a complete consumption of the starting material (PE/Et₂O=10/1), petroleum ether was added to the RM, which caused the precipitation of PPh₃O. The suspension was filtered through Celite® and then concentrated under reduced pressure. The resulting residue was purified by column chromatography (SiO₂, eluting with PE/Et₂O=0/1 to 20/1) providing the title compound Intermediate F2-2 (105 g) as a white solid. ¹H NMR (400 MHz, Chloroform-d) δ [ppm] 3.67 (s, 3H), 3.41 (t, J=7.0 Hz, 2H), 2.31 (t, J=7.6 Hz, 2H), 1.86 (m, 2H), 1.62 (m, 2H), 1.38-1.51 (m, 2H), 1.19-1.37 (m, 18H).

### Step 3: 15-Bromopentadecanoic acid (Intermediate F2-3)

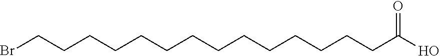 [SMILES: O=C(O)CCCCCCCCCCCCCCBr]

To a solution of methyl 15-bromopentadecanoate (Intermediate F2-2, 105 g, 315 mmol) in THF (1.5 L) was added a solution of lithium hydroxide monohydrate (66.1 g, 1.58 mol) in water (1.5 L). The resulting mixture was stirred at 18° C. for 16 h. Once TLC indicated a complete consumption of the starting material, the RM was diluted with aq. HCl (1M, 200 mL) and concentrated under reduced pressure to remove the THF. The residue was diluted with water (200 mL) and extracted with DCM (3×200 mL). The combined organic layers were dried over Na₂SO₄, filtered, and concentrated under reduced pressure affording the crude title compound Intermediate F2-3 (80.5 g) as a white solid which was used in the next step without further purification. ¹H NMR (400 MHz, Chloroform-d) δ [ppm] 3.41 (t, J=7.0 Hz, 2H), 2.36 (t, J=7.6 Hz, 2H), 1.86 (m, 2H), 1.59-1.69 (m, 2H), 1.39-1.48 (m, 2H), 1.27 (m, 18H).

### Step 4: Benzyl 15-bromopentadecanoate (Intermediate F2-4)

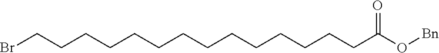 [SMILES: O=C(CCCCCCCCCCCCCCBr)OCc1ccccc1]

To a mixture of crude 15-bromopentadecanoic acid (Intermediate F2-3, 80.5 g) and BnOH (40.6 g, 375 mmol) in DCM (1.5 L) was added EDCl (72.0 g, 375 mmol) and DMAP (3.06 g, 25.0 mmol) in one portion at 18° C. The resulting mixture was stirred at 18° C. for 16 h. Once TLC indicated a complete consumption of the starting material (PE/Et₂O=5/1), the RM was poured into water (1 L) and extracted with DCM (3×300 mL). The combined organic layers were washed with brine (2×500 mL), dried over Na₂SO₄, filtered and concentrated under reduced pressure. The resulting residue was purified by column chromatography (SiO₂, eluting with PE/Et₂O=1/0 to 50/1) providing the title compound Intermediate F2-4 (74.7 g) as a white solid. ¹H NMR (400 MHz, Chloroform-d) δ [ppm] 7.28-7.42 (m, 5H), 5.12 (s, 2H), 3.42 (t, J=6.8 Hz, 2H), 2.36 (t, J=7.6 Hz, 2H), 1.86 (m, 2H), 1.65 (m, 2H), 1.38-1.49 (m, 2H), 1.20-1.35 (m, 18H).

### Step 5: 1,15,29-Tribenzyl 15-(tert-butyl) nonacosane-1,15,15,29-tetracarboxylate (Intermediate F2-5)

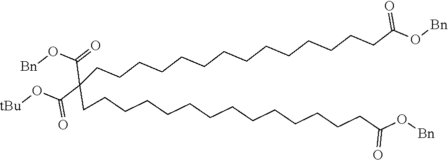 [SMILES: CC(C)(C)OC(=O)C(CCCCCCCCCCCCCCC(=O)OCc1ccccc1)(CCCCCCCCCCCCCCC(=O)OCc1ccccc1)C(=O)OCc1ccccc1]

Two parallel reactions of benzyl 15-bromopentadecanoate (Intermediate F2-4, 100 g scale and 38.7 g scale) with benzyl tert-butyl malonate were performed. The 100 g scale reaction is described up to the combination of the two parallel reactions for purification.

To a mixture of NaH (16.8 g, 422 mmol, 60 wt. %) and NaI (3.31 g, 22.1 mmol) in DMF (1.00 L) was added benzyl tert-butyl malonate (27.6 g, 110 mmol) and benzyl 15-bromopentadecanoate (Intermediate F2-4, 100 g, 243.0 mmol) in DMF (500 mL) at 0° C. The RM was stirred at 0° C. for 1 h, allowed to warm up to 18° C. and then stirred for additional 23 h. The RM was poured into ice water (4.5 L) and extracted with Et₂O (3×1 L). The combined organic layers were washed with brine (2×1 L), dried over Na₂SO₄, filtered, and concentrated under reduced pressure providing a crude residue (100 g). The crude residues from the two parallel reactions (100 g and 39 g, respectively) were combined and purified by column chromatography (SiO₂, eluting with PE/Et₂O=1/0 to 50/1) to provide the title compound Intermediate F2-5 (92.4 g). ¹H NMR (400 MHz, Chloroform-d) δ [ppm] 7.29-7.43 (m, 15H), 5.15 (s, 2H), 5.12 (s, 4H), 2.36 (t, J=7.6 Hz, 4H), 1.84 (t, J=8.4 Hz, 4H), 1.62-1.68 (m, 4H), 1.34 (s, 9H), 1.21-1.32 (m, 44H).

### Step 6: 17-(Benzyloxy)-2-(15-(benzyloxy)-15-oxopentadecyl)-2-((benzyloxy)carbonyl)-17-oxoheptadecanoic acid (Intermediate F2)

To a solution of 1,15,29-tribenzyl 15-(tert-butyl) nonacosane-1,15,15,29-tetracarboxylate (Intermediate F2-5, 92.4 g, 101 mmol) in DCM (1.0 L) was added TFA (115 g, 1.01 mol). The resulting mixture was stirred at 18° C. for 24 h. The RM was diluted with aq. sat. NaHCO₃ solution (0.6 L) at 18° C. and extracted with DCM (3×200 mL). The combined organic layers were washed with brine (1 L), dried over Na₂SO₄, filtered, and concentrated under reduced pressure providing the crude title compound Intermediate F2 (81.8 g), which was used directly in the next step without further purification. ¹H NMR (400 MHz, Chloroform-d) δ [ppm] 7.28-7.46 (m, 15H), 5.24 (s, 2H), 5.12 (s, 4H), 2.36 (t, J=7.6 Hz, 4H), 1.92-2.03 (m, 2H), 1.79-1.90 (m, 2H), 1.59-1.71 (m, 4H), 1.13-1.35 (m, 44H). LCMS Method C3: Rt=1.24 min, MS m/z [M+H]⁺=855.6

### Example 15a: 2-((27-((2,5-Dioxopyrrolidin-1-yl)oxy)-27-oxo-3,6,9,12,15,18,21,24-octaoxaheptacosyl)carbamoyl)-2-undecyltridecanedioic acid (Intermediate rac-F1-Peg8)

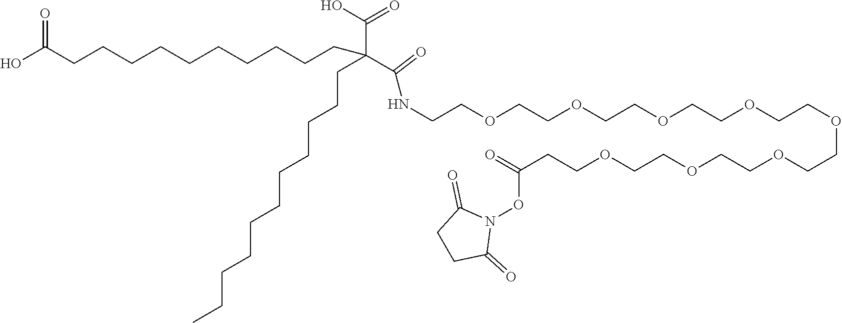 [SMILES: CCCCCCCCCCCC(CCCCCCCCCCC(=O)O)(C(=O)O)C(=O)NCCOCCOCCOCCOCCOCCOCCOCCOCCC(=O)ON1C(=O)CCC1=O]

### Step 1: 1,11-Dibenzyl 11-(2,5-dioxocyclopentyl) docosane-1,11,11-tricarboxylate (Intermediate rac-F1-Peg8-1)

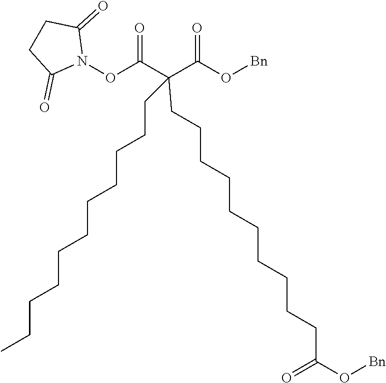 [SMILES: CCCCCCCCCCCC(CCCCCCCCCCC(=O)OCc1ccccc1)(C(=O)OCc1ccccc1)C(=O)ON1C(=O)CCC1=O]

To a 1000 mL 3-neck round bottom flask (fitted with a mechanical stirrer and N₂ inlet) was added 13-(benzyloxy)-2-((benzyloxy)carbonyl)-13-oxo-2-undecyltridecanoic acid (Intermediate rac-F1, 37.7 g, 60.5 mmol), DCM (360 mL), and THF (40 mL). To the resulting homogeneous solution was added N-hydroxysuccinimide (7.31 g, 63.6 mmol) and DCC (14.99 g, 72.6 mmol). Five min after addition, the RM had become a white suspension. The batch was stirred for 6 h at room temperature and then filtered over a pad of Celite®. The pad was washed thoroughly with DCM (2 bed volumes). The combined filtrate and DCM washes were concentrated under reduced pressure, and the residue was dried under high vacuum. The crude product was isolated as a white oil. The crude product was taken up in DCM (˜400 mL) and SiO₂ (75 g) was added. The suspension was concentrated under reduced pressure and the residue dried under high vacuum for 3 h. The batch was purified via column chromatography (750 g SiO₂, eluting with 2% EtOAc/heptane to 35% EtOAc/heptane). The product fractions were combined, concentrated under reduced pressure and dried overnight under high vacuum to provide the title compound Intermediate rac-F1-Peg8-1 as a colorless oil. ¹H NMR (400 MHz, Chloroform-d) δ [ppm] 0.86-0.93 (m, 3H), 1.12-1.21 (m, 2H), 1.21-1.37 (m, 30H), 1.66 (quin, J=7.40 Hz, 2H), 1.89-2.07 (m, 4H), 2.37 (t, J=7.58 Hz, 2H), 2.84 (br s, 4H), 5.13 (s, 2H), 5.25 (s, 2H), 7.30-7.47 (m, 10H).

### Step 2: 14-((Benzyloxy)carbonyl)-3,15-dioxo-1-phenyl-14-undecyl-2,19,22,25,28,31,34,37,40-nonaoxa-16-azatritetracontan-43-oic acid (Intermediate rac-F1-Peg8-2)

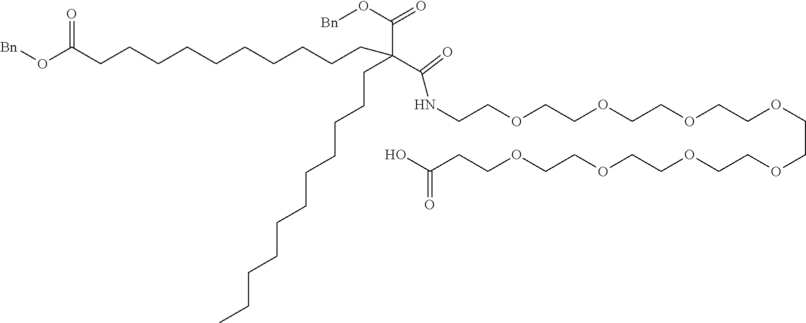 [SMILES: CCCCCCCCCCCC(CCCCCCCCCCC(=O)OCc1ccccc1)(C(=O)NCCOCCOCCOCCOCCOCCOCCOCCOCCC(=O)O)C(=O)OCc1ccccc1]

To a 250 mL round bottom flask (fitted with a magnetic stirrer and N₂ inlet) was added 1,11-dibenzyl 11-(2,5-dioxocyclopentyl) docosane-1,11,11-tricarboxylate (Intermediate rac-F1-Peg8-1, 7.0 g, 9.72 mmol) and DCM (70 mL). To this mixture was added 1-amino-3,6,9,12,15,18,21,24-octaoxaheptacosan-27-oic acid (4.51 g, 10.21 mmol), DIPEA (4.25 mL, 24.31 mmol), and DMAP (0.119 g, 0.972 mmol). The light yellow homogeneous solution was stirred overnight at ambient temperature and then concentrated under reduced pressure to provide a light yellow oil. The light yellow oil was diluted with EtOAc (150 mL) and washed with brine (500 mL). After separation of the layers, the aq. phase was back-extracted 2× with EtOAc (150 mL; then 100 mL). The combined organic phases were dried over Na₂SO₄, filtered over Celite®, and concentrated under reduced pressure. The crude product was purified via column chromatography (330 g SiO₂, eluting with DCM to 10% MeOH/DCM). The product fractions were combined, concentrated under reduced pressure and dried overnight under high vacuum to give the title compound Intermediate rac-F1-Peg8-2. LCMS Method T11: Rt=1.43 min; MS m/z [M+H]+ 1047.0.

### Step 3: 29,39-Dibenzyl 1-(2,5-dioxopyrrolidin-1-yl) 28-oxo-29-undecyl-3,6,9,12,15,18,21,24-octaoxa-27-azanonatriacontane-1,29,39-tricarboxylate (Intermediate rac-F1-Peg8-3)

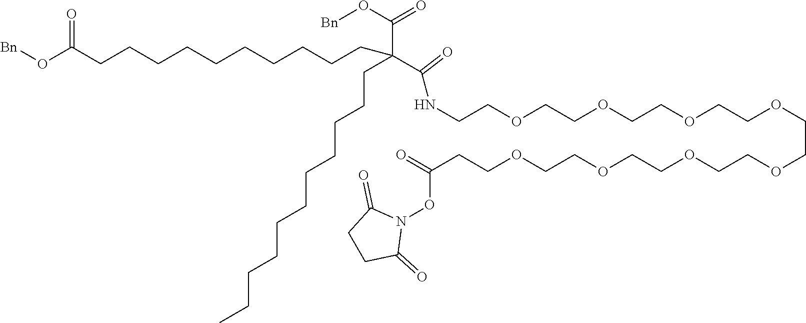 [SMILES: CCCCCCCCCCCC(CCCCCCCCCCC(=O)OCc1ccccc1)(C(=O)NCCOCCOCCOCCOCCOCCOCCOCCOCCC(=O)ON1C(=O)CCC1=O)C(=O)OCc1ccccc1]

To a solution of 14-((benzyloxy)carbonyl)-3,15-dioxo-1-phenyl-14-undecyl-2,19,22,25,28,31,34,37,40-nonaoxa-16-azatritetracontan-43-oic acid (Intermediate rac-F1-Peg8-2, 5.51 g, 5.27 mmol) in DCM (27.5 mL) and THF (27.5 mL) was added DCC (1.412 g, 6.85 mmol) and N-hydroxysuccinimide (0.697 g, 6.06 mmol). After stirring for approximately 10 min, the batch became a thick white suspension. The batch was stirred for 3.75 h at ambient temperature and then concentrated under reduced pressure to provide a white paste. DCM (35 mL) was added to the white paste and the resulting white suspension was stirred for 10 min. The mixture was then filtered over a pad of Celite® and the pad was washed with cold DCM (one bed volume). The combined filtrates were concentrated under reduced pressure and dried overnight under high vacuum to provide the title compound Intermediate rac-F1-Peg8-3 as a colorless oil. LCMS Method T11: Rt=1.45 min; MS m/z [M+H]+ 1144.0.

### Step 4: 2-((27-((2,5-Dioxopyrrolidin-1-yl)oxy)-27-oxo-3,6,9,12,15,18,21,24-octaoxaheptacosyl)carbamoyl)-2-undecyltridecanedioic acid (Intermediate rac-F1-Peg8)

To a 250 mL round bottom flask (fitted with a magnetic stirrer) was added 29,39-dibenzyl 1-(2,5-dioxopyrrolidin-1-yl) 28-oxo-29-undecyl-3,6,9,12,15,18,21,24-octaoxa-27-azanonatriacontane-1,29,39-tricarboxylate (Intermediate rac-F1-Peg8-3, 6.0 g, 5.25 mmol) and THF (70 mL). To this solution was added 10% Pd/C (0.603 g, 0.567 mmol), and the reaction vessel was purged with N₂ and H₂. The batch was then exposed to H₂ (balloon pressure) for 3 h. The reaction vessel was purged with N₂ and the suspension was filtered over a pad of Celite®. The pad was then washed thoroughly with THF. The combined filtrates were concentrated under reduced pressure, and dried overnight under high vacuum to provide the title compound Intermediate rac-F1-Peg8 as a colorless oil. LCMS Method T11: Rt=0.91 min; MS m/z [M+H]+ 963.8.

### Example 16a: 2-((39-((2,5-Dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate rac-F1-Peg12)

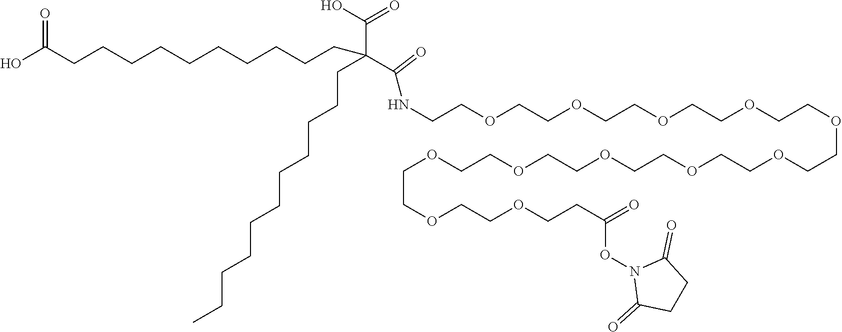 [SMILES: CCCCCCCCCCCC(CCCCCCCCCCC(=O)O)(C(=O)O)C(=O)NCCOCCOCCOCCOCCOCCOCCOCCOCCOCCOCCOCCOCCC(=O)ON1C(=O)CCC1=O]

### Step 1: Dibenzyl 2-(chlorocarbonyl)-2-undecyltridecanedioate (Intermediate rac-F1-Peg12-1)

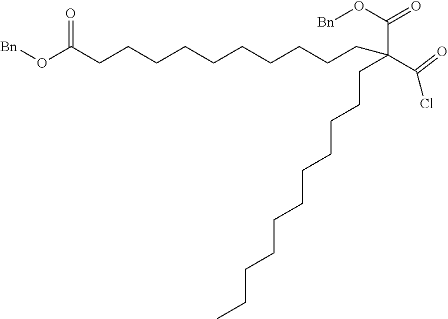 [SMILES: CCCCCCCCCCCC(CCCCCCCCCCC(=O)OCc1ccccc1)(C(=O)Cl)C(=O)OCc1ccccc1]

To solution of 13-(benzyloxy)-2-((benzyloxy)carbonyl)-13-oxo-2-undecyltridecanoic acid (Intermediate rac-F1, 5 g, 8.03 mmol) in DCM (25 mL) under a N₂ atmosphere at RT was added DMF (0.012 mL, 0.161 mmol) followed by a dropwise addition of oxalyl chloride (0.913 mL, 10.4 mmol) over 5 min. Upon addition of oxalyl chloride, the mixture became cloudy and gas evolution started. The RM stirred at RT for 2.25 h. The mixture was then concentrated under reduced pressure to give a colorless oil. To this oil was added 6 mL of heptane and the resulting mixture concentrated under reduced pressure to give a colorless oil with some solid particles (5.8 g). The oil was then diluted with DCM (50 mL) and the insoluble particles were filtered. The resulting DCM filtrate containing the title compound Intermediate rac-F1-Peg12-1 was used in the next step without further purification.

### Step 2: 14-((Benzyloxy)carbonyl)-3,15-dioxo-1-phenyl-14-undecyl-2,19,22,25,28,31,34,37,40,43,46,49,52-tridecaoxa-16-azapentapentacontan-55-oic acid (Intermediate rac-F1-Peg12-2)

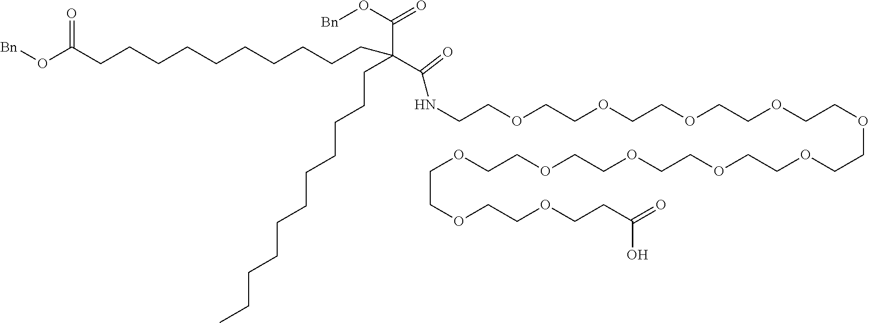 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCC(=O)O)C(=O)OCc1ccccc1]

1-amino-3,6,9,12,15,18,21,24,27,30,33,36-dodecaoxanonatriacontan-39-oic acid (5.5 g, 8.90 mmol) in DCM (58 mL) under a N₂ atmosphere at RT was treated with DIPEA (3.11 mL, 17.81 mmol). The resulting suspension slowly became a clear solution after stirring for 10 min and sonicating for 5 min at RT. To this solution was then added a solution of dibenzyl 2-(chlorocarbonyl)-2-undecyltridecanedioate (Intermediate rac-F1-Peg12-1, 5.14 g, 8.01 mmol) in DCM (50 mL) dropwise over 30 min. The RM was stirred at RT for 3 h, treated with anhydrous MgSO₄ (6.6 g) and then stirred for 0.5 h. Then Dowex 50WX2 hydrogen form 50-100 mesh resin (6.6 g) was added in one portion and stirring was continued at RT for an additional 0.5 h. The mixture was then filtered and the filter cake washed with DCM (50 mL). The combined filtrate and DCM wash were concentrated under reduced pressure to give an off-white oil. The oil was dried under high vacuum overnight at RT to afford a gel. This crude gel was diluted with EtOAc (250 mL) and washed with 0.5M HCl (aq.) (3×100 mL). The combined aq. HCl washes were extracted 1× with EtOAc (200 mL). The combined EtOAc layers were dried over MgSO₄, filtered, and concentrated under reduced pressure to yield 10.7 g of the title compound Intermediate rac-F1-Peg12-2 as a colorless oil. The product was used in the next step without further purification. LCMS Method T12: Rt 3.01 min; MS m/z [M+H]⁺=1223.3.

### Step 3: 41,51-Dibenzyl 1-(2,5-dioxopyrrolidin-1-yl) 40-oxo-41-undecyl-3,6,9,12,15,18,21,24,27,30,33,36-dodecaoxa-39-azahenpentacontane-1,41,51-tricarboxylate (Intermediate rac-F1-Peg12-3)

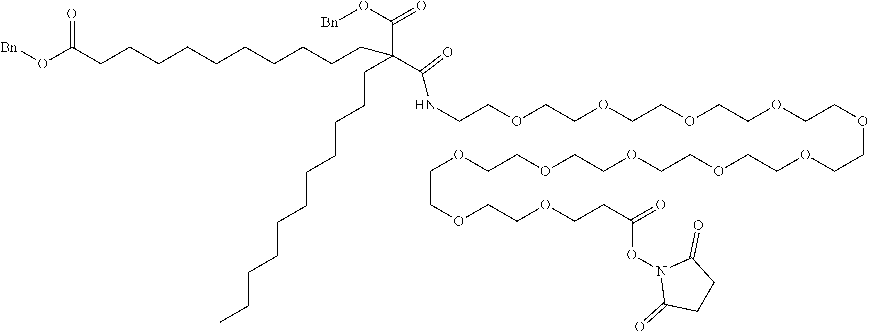 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCC(=O)ON1C(=O)CCC1=O)C(=O)OCc1ccccc1]

To a stirring solution of 14-((benzyloxy)carbonyl)-3,15-dioxo-1-phenyl-14-undecyl-2,19,22,25,28,31,34,37,40,43,46,49,52-tridecaoxa-16-azapentapentacontan-55-oic acid (Intermediate rac-F1-Peg12-2, 9.67 g, 0.792 mmol) in DCM (65 mL) at RT was added TEA (0.329 mL, 0.160 mmol) followed by the addition of DSC (2.86 g, 11.15 mmol) in 4 portions over 2 h (0.5 eq at the beginning, 0.5 eq after 0.5 h, 0.2 eq after 0.5 h, and 0.1 eq after 1 h). The RM was then stirred at RT for ˜16 h. Dowex 50WX2 hydrogen form 50-100 mesh resin (2.57 g) was added to the RM and stirring was continued for 0.5 h. MgSO₄ (2.57 g) was then added and the resulting suspension was stirred for an additional 0.5 h. The mixture was filtered and the filter cake was washed with DCM (50 mL). The combined filtrate and DCM wash were concentrated under reduced pressure to afford a thick, pale yellow oil. This crude product was diluted with DCM (15 mL) and purified on an Isco RediSep® 150 g silica cartridge eluting with a 0 to 10% MeOH/DCM gradient. Product fractions were collected and concentrated under reduced pressure to a minimal volume. To this residue was added heptane (50 mL) and the solution concentrated under reduced pressure. The residue was treated a second time with heptane (50 mL), concentrated, and dried under high vacuum to give 10.7 g of the title compound Intermediate rac-F1-Peg12-3 as a colorless oil. LCMS Method T12: Rt 3.09 min; MS m/z [M+H]⁺=1321.3.

### Step 4: 2-((39-((2,5-Dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate rac-F1-Peg12)

To a solution of 41,51-dibenzyl 1-(2,5-dioxopyrrolidin-1-yl) 40-oxo-41-undecyl-3,6,9,12,15,18,21,24,27,30,33,36-dodecaoxa-39-azahenpentacontane-1,41,51-tricarboxylate (Intermediate rac-F1-Peg12-3, 10.7 g, 8.11 mmol) in THF (150 mL) under N₂ was added anhydrous MgSO₄ (1.07 g) followed by Pd/C (10% on activated charcoal, 1.07 g, 1.00 mmol). The suspension was placed under an atmosphere of H₂ and stirred for 16 h. The RM was purged with N₂ and filtered through a pre-loaded celite* filter that was pre-wetted with THF. The solid was rinsed 4× with THF. The combined filtrate and THF washes were concentrated under reduced pressure to afford 9.9 g of the title compound Intermediate rac-F1-Peg12 as a viscous oil. LCMS Method T11: Rt 0.93 min; MS m/z [M+H]⁺=1140.2.

### Example 17a: (R)-2-((39-((2,5-Dioxopyrrolidin-1-yl)oxy)-39-oxo-3,6,9,12,15,18,21,24,27,30,33,36-dodecaoxanonatriacontyl)carbamoyl)-2-undecyltridecanedioic acid and(S)-2-((39-((2,5-dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate ent-F1-Peak1-Peg12 and Intermediate ent-F1-Peak2-Peg12)

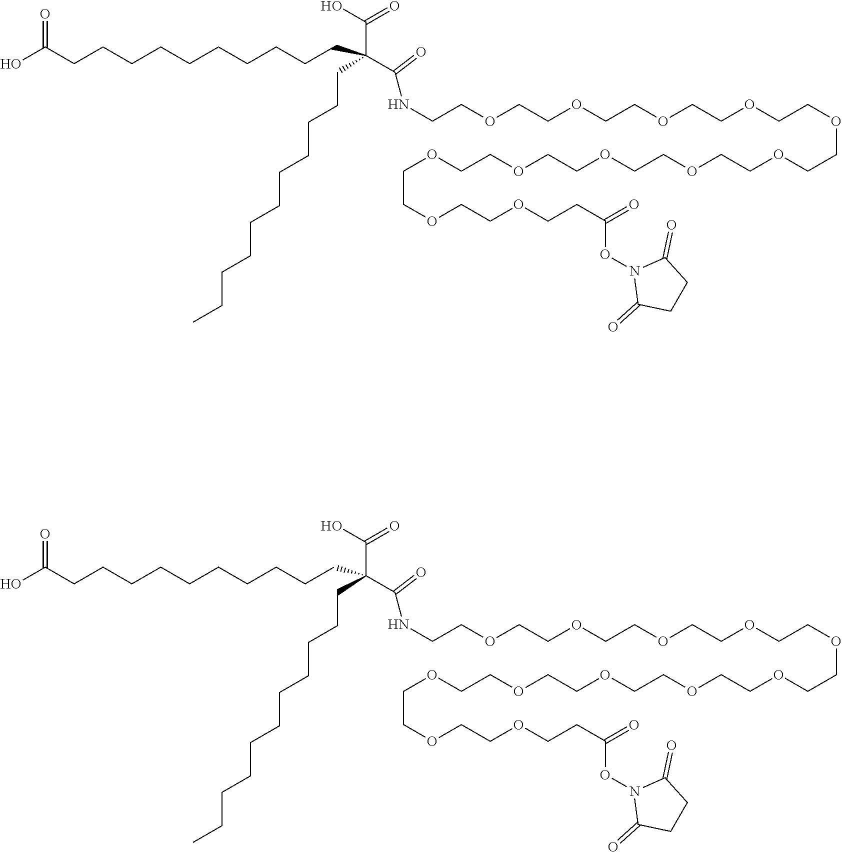 [SMILES: CCCCCCCCCCC[C@](CCCCCCCCCCC(=O)O)(C(=O)O)C(=O)NCCOCCOCCOCCOCCOCCOCCOCCOCCOCCOCCOCCOCCC(=O)ON1C(=O)CCC1=O ; CCCCCCCCCCC[C@@](CCCCCCCCCCC(=O)O)(C(=O)O)C(=O)NCCOCCOCCOCCOCCOCCOCCOCCOCCOCCOCCOCCOCCC(=O)ON1C(=O)CCC1=O]

### Step 1: Dibenzyl (R)-2-(chlorocarbonyl)-2-undecyltridecanedioate and Dibenzyl (S)-2-(chlorocarbonyl)-2-undecyltridecanedioate (Intermediate ent-F1-Peak1-Peg12-1 and Intermediate ent-F1-Peak2-Peg12-1)

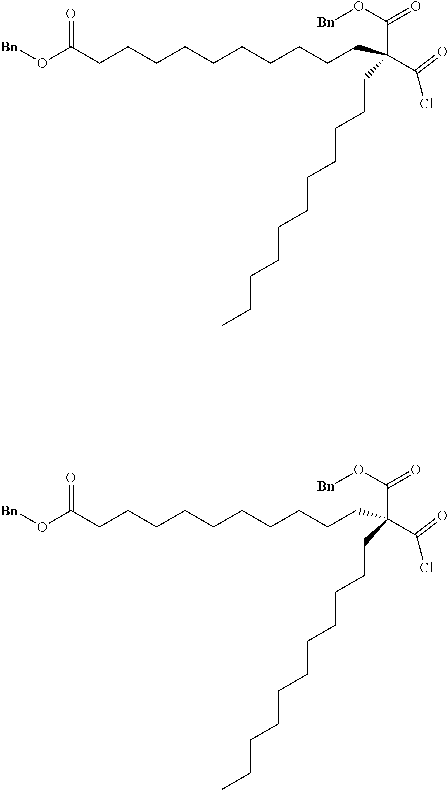 [SMILES: CCCCCCCCCCC[C@@](CCCCCCCCCCC(=O)OCc1ccccc1)(C(=O)Cl)C(=O)OCc1ccccc1 ; CCCCCCCCCCC[C@](CCCCCCCCCCC(=O)OCc1ccccc1)(C(=O)Cl)C(=O)OCc1ccccc1]

### Step 1-1: Intermediate ent-F1-Peak1-Peg12-1

To a solution of Intermediate ent-F1-Peak1 (3.04 g, 4.88 mmol) in DCM (10 mL) was added DMF (7.56 μL, 0.098 mmol) and oxalyl chloride (0.56 mL, 6.34 mmol). The RM was allowed to stir for 1 h at RT and then concentrated under reduced pressure. To the resulting residue was added heptane (10 mL) and the mixture was concentrated under reduced pressure. To this resulting residue was added DCM (10 mL), the resulting mixture was filtered and the filtrate containing the title compound Intermediate ent-F1-Peak1-Peg12-1 was used immediately in the next step without further purification.

### Step 1-2: Intermediate ent-F1-Peak2-Peg12-1

To a solution of Intermediate ent-F1-Peak2 (3.07 g, 4.93 mmol) in DCM (10 mL) was added DMF (7.63 μL, 0.099 mmol) and oxalyl chloride (0.56 mL, 6.41 mmol). The RM was allowed to stir for 2 h at RT and then concentrated under reduced pressure. To the resulting residue was added heptane (10 mL) and the resulting mixture was concentrated under reduced pressure. To this resulting residue was added DCM (10 mL), the resulting mixture was filtered and the filtrate containing the title compound Intermediate ent-F1-Peak2-Peg12-1 was used immediately in the next step without further purification.

### Step 2: (R)-14-((Benzyloxy)carbonyl)-3,15-dioxo-1-phenyl-14-undecyl-2,19,22,25,28,31,34,37,40,43,46,49,52-tridecaoxa-16-azapentapentacontan-55-oic acid and (S)-14-((benzyloxy)carbonyl)-3,15-dioxo-1-phenyl-14-undecyl-2,19,22,25,28,31,34,37,40,43,46,49,52-tridecaoxa-16-azapentapentacontan-55-oic acid (Intermediate ent-F1-Peak1-Peg12-2 and Intermediate ent-F1-Peak2-Peg12-2)

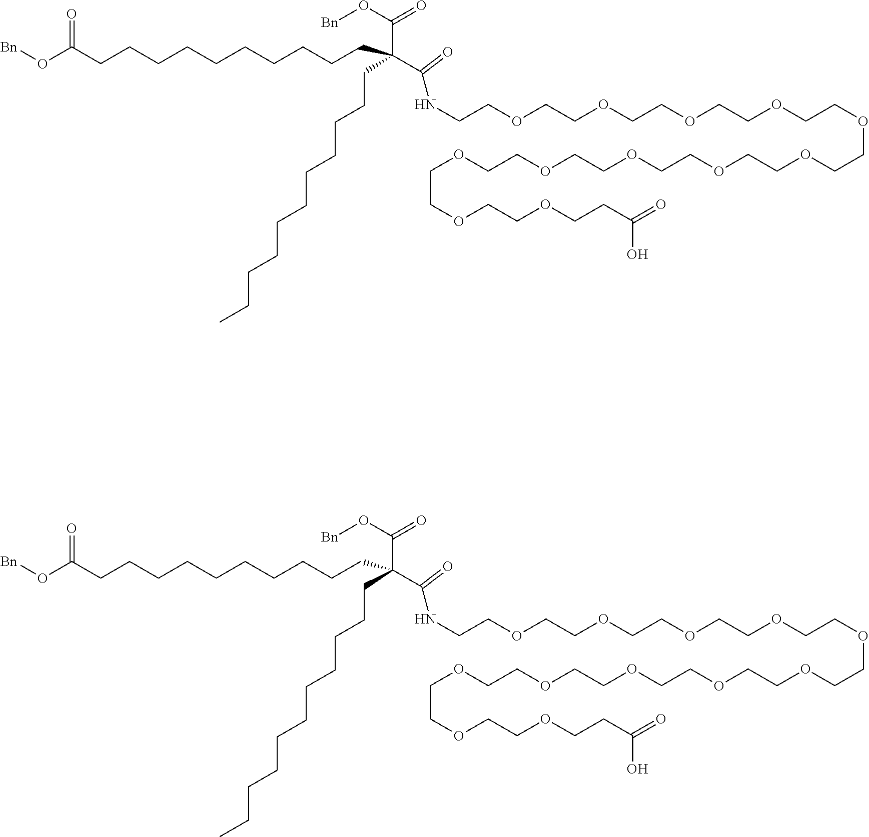 [SMILES: CCCCCCCCCCC[C@@](CCCCCCCCCCC(=O)OCc1ccccc1)(C(=O)NCCOCCOCCOCCOCCOCCOCCOCCOCCOCCOCCOCCOCCC(=O)O)C(=O)OCc1ccccc1 ; CCCCCCCCCCC[C@](CCCCCCCCCCC(=O)OCc1ccccc1)(C(=O)NCCOCCOCCOCCOCCOCCOCCOCCOCCOCCOCCOCCOCCC(=O)O)C(=O)OCc1ccccc1]

### Step 2-1: Intermediate ent-F1-Peak1-Peg12-2

To the filtrate containing Intermediate ent-F1-Peak1-Peg12-1 was added DIPEA (1.58 mL, 9.04 mmol) and 1-amino-3,6,9,12,15,18,21,24,27,30,33,36-dodecaoxanonatriacontan-39-oic acid (2.93 g, 4.75 mmol). After stirring the RM for 1 h at RT, MgSO₄ (5 g) was added and stirring was continued for 30 min. Dowex resin WX4 (100 mesh, 5 g) was added and stirring was continued for an additional 30 min. The resulting mixture was filtered, rinsed with DCM, and the filtrate was concentrated under reduced pressure. The resulting crude residue containing the title compound Intermediate ent-F1-Peak1-Peg12-2 was used directly in the next step without further purification. LCMS Method T12: Rt=3.07 min; MS m/z [M+H]⁺=1223.3.

### Step 2-2: Intermediate ent-F1-Peak2-Peg12-2

To the filtrate containing Intermediate ent-F1-Peak2-Peg12-1 was added DIPEA (1.62 mL, 9.26 mmol) and 1-amino-3,6,9,12,15,18,21,24,27,30,33,36-dodecaoxanonatriacontan-39-oic acid (3.0 g, 4.75 mmol). After stirring the RM for 1 h at RT, MgSO₄ (5 g) was added and stirring was continued for 30 min. Dowex resin WX4 (100 mesh, 5 g) was added and stirring was continued for additional 30 min. The resulting mixture was filtered, rinsed with DCM, and the filtrate was concentrated under reduced pressure. The resulting crude residue containing the title compound Intermediate ent-F1-Peak2-Peg12-2 was used directly in the next step without further purification. LCMS Method T12: Rt=3.07 min; MS m/z [M+H]⁺=1223.3.

### Step 3: 41,51-Dibenzyl 1-(2,5-dioxopyrrolidin-1-yl) (R)-40-oxo-41-undecyl-3,6,9,12,15,18,21,24,27,30,33,36-dodecaoxa-39-azahenpentacontane-1,41,51-tricarboxylate and 41,51-dibenzyl 1-(2,5-dioxopyrrolidin-1-yl) (S)-40-oxo-41-undecyl-3,6,9,12,15,18,21,24,27,30,33,36-dodecaoxa-39-azahenpentacontane-1,41,51-tricarboxylate (Intermediate ent-F1-Peak1-Peg12-3 and Intermediate ent-F1-Peak2-Peg12-3)

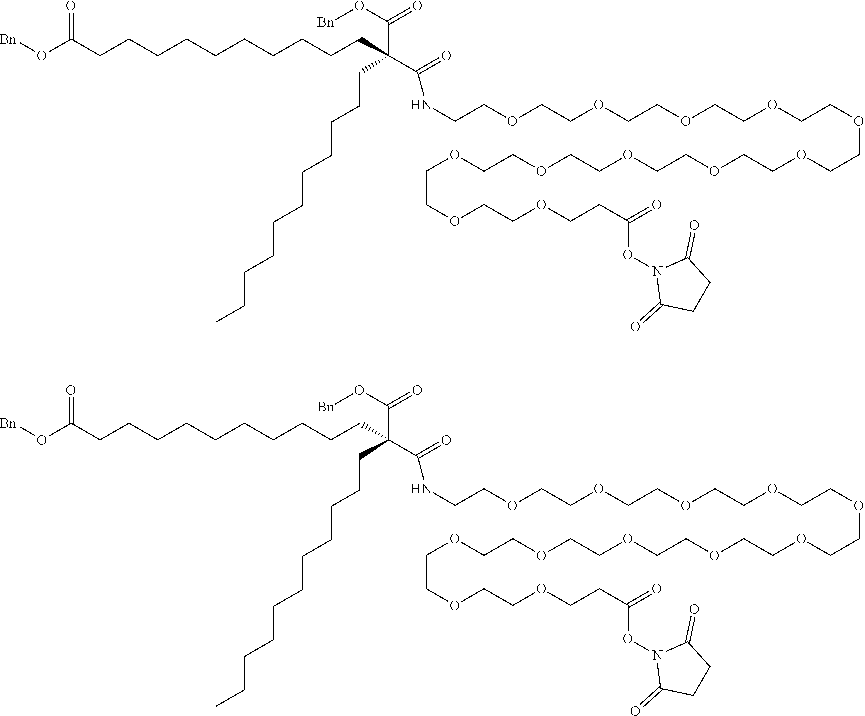 [SMILES: CCCCCCCCCCC[C@@](CCCCCCCCCCC(=O)OCc1ccccc1)(C(=O)NCCOCCOCCOCCOCCOCCOCCOCCOCCOCCOCCOCCOCCC(=O)ON1C(=O)CCC1=O)C(=O)OCc1ccccc1 ; CCCCCCCCCCC[C@](CCCCCCCCCCC(=O)OCc1ccccc1)(C(=O)NCCOCCOCCOCCOCCOCCOCCOCCOCCOCCOCCOCCOCCC(=O)ON1C(=O)CCC1=O)C(=O)OCc1ccccc1]

### Step 3-1: Intermediate ent-F1-Peak1-Peg12-3

To a solution of the crude residue containing Intermediate ent-F1-Peak1-Peg12-2 (5.53 g, 4.52 mmol) and TEA (0.126 mL, 0.905 mmol) in DCM (15 mL) was added DSC (1.51 g, 5.88 mmol) and the resulting mixture was allowed to stir for 16 h at RT under an N₂ atmosphere. To the RM was added Dowex resin WX4 (100 mesh, 1 g) and stirring was continued for 30 min. To the mixture was added MgSO₄ (5 g) and stirring was continued for 30 min. The mixture was then filtered, rinsed with DCM, and the filtrate was concentrated under reduced pressure. The resulting crude residue containing the title compound Intermediate ent-F1-Peak1-Peg12-3 (6.31 g) was used directly in the next step without further purification. LCMS Method T11: Rt=1.53 min; MS m/z [M+H]⁺=1320.4.

### Step 3-2: Intermediate ent-F1-Peak2-Peg12-3

To a solution of the crude residue containing Intermediate ent-F1-Peak2-Peg12-2 (5.53 g, 4.52 mmol) and TEA (0.126 mL, 0.905 mmol) in DCM (15 mL) was added DSC (1.51 g, 5.88 mmol) and the resulting mixture was allowed to stir for 16 h at RT under an N₂ atmosphere. To the RM was added Dowex resin WX4 (100 mesh, 1 g) and stirring was continued for 30 min. To the mixture was added MgSO₄ (5 g) and stirring was continued for 30 min. The mixture was then filtered, rinsed with DCM, and the filtrate was concentrated under reduced pressure. The resulting crude residue containing the title compound Intermediate ent-F1-Peak2-Peg12-3 (6.24 g) was used directly in the next step without further purification. LCMS Method T11: Rt=1.52 min; MS m/z [M+H]⁺=1320.4.

### Step 4: (R)-2-((39-((2,5-Dioxopyrrolidin-1-yl)oxy)-39-oxo-3,6,9,12,15,18,21,24,27,30,33,36-dodecaoxanonatriacontyl)carbamoyl)-2-undecyltridecanedioic acid and (S)-2-((39-((2,5-dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate ent-F1-Peak1-Peg12 and Intermediate ent-F1-Peak2-Peg12)

### Step 4-1: Intermediate ent-F1-Peak1-Peg12

A mixture of the crude residue containing Intermediate ent-F1-Peak1-Peg12-3 (5.97 g, 4.52 mmol) and MgSO₄ (0.544 g, 4.52 mmol) in THF (50 mL) was degassed with N₂. To the mixture was added Pd/C (10 wt. %, 481 mg, 0.452 mmol) and the resulting mixture was purged with H₂ and allowed to stir at RT for 18 h. The mixture was then filtered through Celite®, washed with MeOH, and the filtrate was concentrated under reduced pressure providing the crude title compound Intermediate ent-F1-Peak1-Peg12 (6.22 g) which was used in the next step without further purification. LCMS Method T11: Rt=0.94 min; MS m/z [M+H]⁺=1140.7.

### Step 4-2: Intermediate ent-F1-Peak2-Peg12

A mixture of the crude residue containing Intermediate ent-F1-Peak2-Peg12-3 (5.97 g, 4.52 mmol) and MgSO₄ (0.544 g, 4.52 mmol) in THF (50 mL) was degassed with N₂. To the mixture was added Pd/C (10 wt. %, 481 mg, 0.452 mmol) and the resulting mixture was purged with H₂ and allowed to stir at RT for 18 h. The mixture was then filtered through Celite®, washed with MeOH, and the filtrate was concentrated under reduced pressure providing the crude title compound Intermediate ent-F1-Peak2-Peg12 (6.16 g) which was used in the next step without further purification. LCMS Method T11: Rt=0.94 min; MS m/z [M+H]⁺=1140.7.

### Example 18a: 2-((75-((2,5-Dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)-2-undecyltridecanedioic acid (Intermediate rac-F1-Peg24)

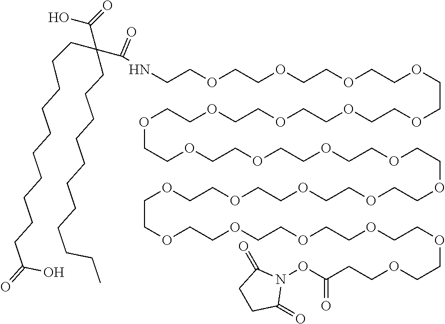 [SMILES: CCCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O]

### Step 1: 14-((Benzyloxy)carbonyl)-3,15-dioxo-1-phenyl-14-undecyl-2,19,22,25,28,31,34,37,40,43,46,49,52,55,58,61,64,67,70,73,76,79,82,85,88-pentacosaoxa-16-azahennonacontan-91-oic acid (Intermediate rac-F1-Peg24-1)

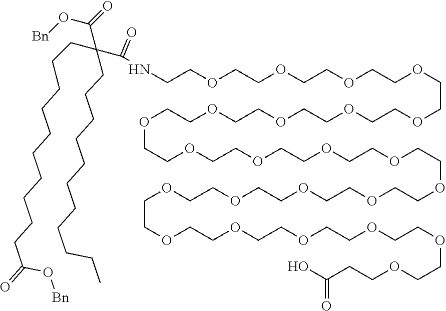 [SMILES: CCCCCCCCCCCC(CCCCCCCCCCC(=O)OCc1ccccc1)(C(=O)NCCOCCOCCOCCOCCOCCOCCOCCOCCOCCOCCOCCOCCOCCOCCOCCOCCOCCOCCOCCOCCOCCOCCOCCOCCC(=O)O)C(=O)OCc1ccccc1]

Step 1a: To a flask were added 13-(benzyloxy)-2-((benzyloxy)carbonyl)-13-oxo-2-undecyltridecanoic acid (Intermediate rac-F1, 640 g, 1.03 mol), DCM (8.3 kg), and DMF (3 g). The resulting mixture was stirred at 25° C. and oxalyl chloride (170 g, 1.34 mol) was then added dropwise. The RM was stirred for another 2 to 3 h. Concentration of the RM and solvent swap with heptane gave a crude mixture of the active acyl chloride (691 g) to which DCM (8.5 kg) was added to form a solution and was used directly in the next step.

Step 1b: To a stirring mixture of 1-amino-3, 6, 9, 12, 15, 18, 21, 24, 27, 30, 33, 36, 39, 42, 45, 48, 51, 54, 57, 60, 63, 66, 69, 72-tetracosaoxapentaheptacontan-75-oic acid (900 g, 0.79 mol), DCM (6.0 kg), and DIPEA (203 g, 1.57 mol) at 25° C. was added the crude acyl chloride solution from Step 1a dropwise (6.76 kg, 0.75 mol, 7.1% pure). The RM was stirred for 1-2 h and then acidic resin (1.3 kg) was added. After stirring for 30 min, the mixture was filtered and MgSO₄ (1.3 kg) added to the filtrate. Stirring was continued for 30 min and then filtered. The filtrate was concentrated under reduced pressure to provide a crude residue. The residue was purified over Al₂O₃ with mobile phase including MTBE, DCM, MeOH to provide the title compound Intermediate rac-F1-Peg24-1 (960 g). ¹H NMR (400 MHz, Chloroform-d) δ [ppm] 0.86-0.93 (m, 3H), 0.93-1.04 (m, 2H), 1.19 (br s, 15H), 1.23-1.37 (m, 15H), 1.61-1.68 (m, 2H), 1.78 (td, J=12.44, 4.34 Hz, 2H), 1.92-2.05 (m, 2H), 2.37 (t, J=7.58 Hz, 2H), 2.62 (t, J=6.05 Hz, 2H), 3.49 (dd, J=6.72, 2.32 Hz, 2H), 3.52-3.59 (m, 2H), 3.59-3.73 (m, 92H), 3.80 (t, J=6.05 Hz, 2H), 5.13 (s, 2H), 5.18 (s, 2H), 7.31-7.42 (m, 10H), 8.09 (t, J=5.26 Hz, 1H). LCMS Method C6: Rt=15.75 min/18.0 min, MS m/z [M+H]+ 1751.1.

### Step 2: 77,87-Dibenzyl 1-(2,5-dioxopyrrolidin-1-yl) 76-oxo-77-undecyl-3,6,9,12,15,18,21,24,27,30,33,36,39,42,45,48,51,54,57,60,63,66,69,72-tetracosaoxa-75-azaheptaoctacontane-1,77,87-tricarboxylate (Intermediate rac-F1-Peg24-2)

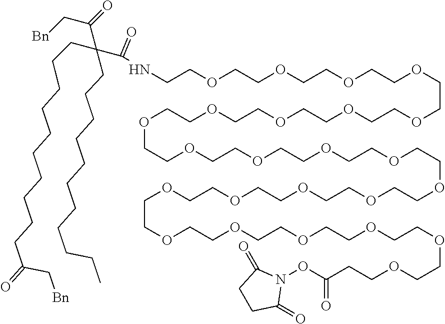 [SMILES: CCCCCCCCCCCC(CCCCCCCCCCC(=O)CCc1ccccc1)(C(=O)CCc1ccccc1)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O]

To the solution of 14-((benzyloxy)carbonyl)-3,15-dioxo-1-phenyl-14-undecyl-2,19,22,25,28,31,34,37,40,43,46,49,52,55,58,61,64,67,70,73,76,79,82,85,88-pentacosaoxa-16-azahennonacontan-91-oic acid (Intermediate rac-F1-Peg24-1, 920 g, 0.53 mol) in DCM (6.1 kg) was added TEA (11 g, 0.11 mol). The resulting mixture was stirred to provide a clear solution, then DSC (161 g, 0.63 mol) was added and the RM was stirred at 25° C. for 2 h. Acidic resin (180 g) was added and this mixture was stirred for an additional 30 min. MgSO₄ (180 g) was added into the mixture and stirring was continued for 30 min. The mixture was then filtered to provide a clear light yellow solution. Concentration of the filtrate under reduced pressure gave crude Intermediate rac-F1-Peg24-2, which was used directly in the next step without further purification. LCMS Method C6: Rt=15.75 min/18.0 min, MS m/z [M+H]+ 1848.2.

### Step 3: 2-((75-((2,5-Dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)-2-undecyltridecanedioic acid (Intermediate rac-F1-Peg24)

To a hydrogenation reactor was added 77,87-dibenzyl 1-(2,5-dioxopyrrolidin-1-yl) 76-oxo-77-undecyl-3,6,9,12,15,18,21,24,27,30,33,36,39,42,45,48,51,54,57,60,63,66,69,72-tetracosaoxa-75-azaheptaoctacontane-1,77,87-tricarboxylate (Intermediate rac-F1-Peg24-2, 986 g, 0.48 mol, 90% pure), THF (7.6 kg), and Pd/C (10 wt. %, 110 g). Then MgSO₄ (110 g) was added. The resulting mixture was purged with N₂ and then placed under an H₂ atmosphere. The RM was stirred at 25° C. for 3-24 h. After complete consumption of starting material, more MgSO₄ (220 g) was added and the RM was stirred for an additional 30 min and then filtered. The filter cake was washed with THF. The filtrate and THF wash were combined and concentrated under reduced pressure to provide the title compound Intermediate rac-F1-Peg24 (800 g). ¹H NMR (400 MHz, Chloroform-d) δ [ppm] 0.84-0.94 (m, 3H), 1.17 (br s, 2H), 1.21-1.39 (m, 30H), 1.57-1.68 (m, 2H), 1.69-1.80 (m, 2H), 1.97-2.10 (m, 2H), 2.34 (t, J=7.21 Hz, 2H), 2.86 (s, 4H), 2.92 (t, J=6.48 Hz, 2H), 3.51-3.73 (m, 96H), 3.87 (t, J=6.48 Hz, 2H), 7.45 (t, J=4.46 Hz, 1H). LCMS Method C6: Rt=14.00 min/18.0 min, MS m/z [M+H]+ 1668.0.

### Example 19a: 15-((39-((2,5-Dioxopyrrolidin-1-yl)oxy)-39-oxo-3,6,9,12,15,18,21,24,27,30,33,36-dodecaoxanonatriacontyl)carbamoyl)nonacosane-1,15,29-tricarboxylic acid (Intermediate F2-Peg12)

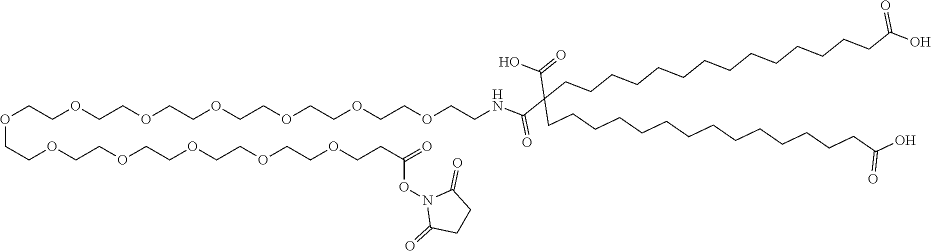 [SMILES: O=C(O)CCCCCCCCCCCCCCC(CCCCCCCCCCCCCCC(=O)O)(C(=O)O)C(=O)NCCOCCOCCOCCOCCOCCOCCOCCOCCOCCOCCOCCOCCC(=O)ON1C(=O)CCC1=O]

### Step 1: 1,15,29-Tribenzyl 15-(2,5-dioxopyrrolidin-1-yl) nonacosane-1,15,15,29-tetracarboxylate (Intermediate F2-Peg12-1)

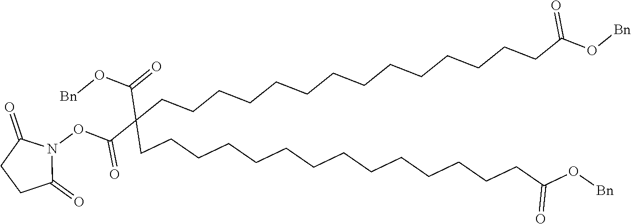 [SMILES: O=C(CCCCCCCCCCCCCCC(CCCCCCCCCCCCCCC(=O)OCc1ccccc1)(C(=O)OCc1ccccc1)C(=O)ON1C(=O)CCC1=O)OCc1ccccc1]

To a solution of crude 17-(benzyloxy)-2-(15-(benzyloxy)-15-oxopentadecyl)-2-((benzyloxy)carbonyl)-17-oxoheptadecanoic acid (Intermediate F2, 81.6 g) and 1-hydroxypyrrolidine-2,5-dione (13.1 g, 114 mmol) in DCM (800 mL) was added DCC (1M in DCM, 143 mL). The resulting mixture was stirred at 20° C. for 12 h, and then filtered and concentrated under reduced pressure. The resulting residue was purified by column chromatography (SiO₂, eluting with PE/Et₂O=1/0 to 5/1) affording the title compound Intermediate F2-Peg12-1 (44.1 g) as a white solid. ¹H NMR (400 MHz, Chloroform-d) δ [ppm] 7.30-7.44 (m, 15H), 5.24 (s, 2H), 5.12 (s, 4H), 2.82 (d, J=2.0 Hz, 4H), 2.36 (t, J=7.6 Hz, 4H), 1.95-2.04 (m, 4H), 1.59-1.70 (m, 4H), 1.18-1.36 (m, 44H). LCMS Method C3: Rt=1.24 min, MS m/z [M+Na]⁺=974.6.

### Step 2: 18-(15-(Benzyloxy)-15-oxopentadecyl)-18-((benzyloxy)carbonyl)-3,19-dioxo-1-phenyl-2,23,26,29,32,35,38,41,44,47,50,53,56-tridecaoxa-20-azanonapentacontan-59-oic acid (Intermediate F2-Peg12-2)

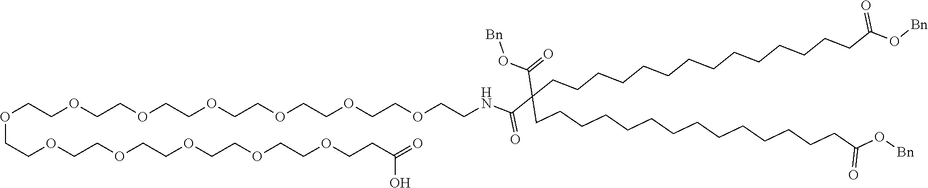 [SMILES: O=C(O)CCOCCOCCOCCOCCOCCOCCOCCOCCOCCOCCOCCOCCNC(=O)C(CCCCCCCCCCCCCCC(=O)OCc1ccccc1)(CCCCCCCCCCCCCCC(=O)OCc1ccccc1)C(=O)OCc1ccccc1]

To a solution of 1,15,29-tribenzyl 15-(2,5-dioxopyrrolidin-1-yl) nonacosane-1,15,15,29-tetracarboxylate (Intermediate F2-Peg12-1, 20.0 g, 20.9 mmol), 1-amino-3,6,9,12,15,18,21,24,27,30,33,36-dodecaoxanonatriacontan-39-oic acid (14.2 g, 23.0 mmol) and DMAP (256 mg, 2.10 mmol) in DCM (200 mL) was added DIPEA (4.06 g, 31.4 mmol). The resulting mixture was stirred at 25° C. for 12 h. The RM was washed with aq. HCl (0.5M, 2×50 mL), and brine (50 mL), dried over Na₂SO₄, filtered and concentrated under reduced pressure providing the crude title compound Intermediate F2-Peg12-2 (25.8 g) as a yellow oil, which was directly used in the next step without further purification. ¹H NMR (400 MHz, Chloroform-d) 5 [ppm] 7.29-7.40 (m, 15H), 5.16 (s, 2H), 5.11 (s, 4H), 3.79-3.71 (m, 2H), 3.59-3.69 (m, 44H), 3.52-3.56 (m, 2H), 3.45-3.51 (m, 2H), 2.56 (s, 2H), 2.35 (t, J=7.6 Hz, 4H), 1.90-2.03 (m, 2H), 1.71-1.83 (m, 2H), 1.58-1.69 (m, 4H), 1.12-1.36 (m, 44H). LCMS Method C3: Rt=1.19 min, MS m/z [M+H]⁺=1455.0.

### Step 3: 41,55-Dibenzyl 1-(2,5-dioxopyrrolidin-1-yl) 41-(15-(benzyloxy)-15-oxopentadecyl)-40-oxo-3,6,9,12,15,18,21,24,27,30,33,36-dodecaoxa-39-azapentapentacontane-1,41,55-tricarboxylate (Intermediate F2-Peg12-3)

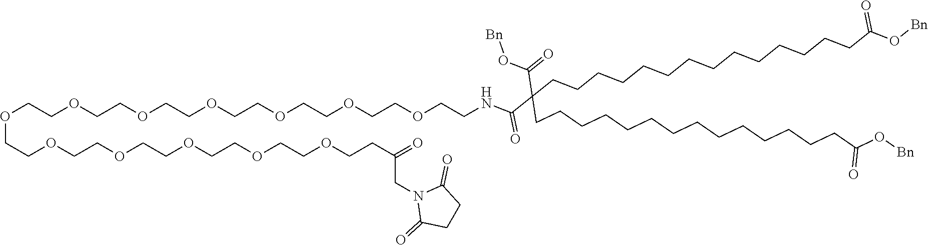 [SMILES: O=C(CCOCCOCCOCCOCCOCCOCCOCCOCCOCCOCCOCCOCCNC(=O)C(CCCCCCCCCCCCCCC(=O)OCc1ccccc1)(CCCCCCCCCCCCCCC(=O)OCc1ccccc1)C(=O)OCc1ccccc1)CN1C(=O)CCC1=O]

To a solution of crude 18-(15-(benzyloxy)-15-oxopentadecyl)-18-((benzyloxy)carbonyl)-3,19-dioxo-1-phenyl-2,23,26,29,32,35,38,41,44,47,50,53,56-tridecaoxa-20-azanonapentacontan-59-oic acid (Intermediate F2-Peg12-2, 25.8 g) and 1-hydroxypyrrolidine-2,5-dione (2.45 g, 21.2 mmol) in DCM (250 mL) was added DCC (1M, 26.6 mL). The resulting mixture was stirred at 25° C. for 12 h. The RM was diluted with EtOAc (500 mL), filtered, and concentrated under reduced pressure. The crude residue was purified by column chromatography (SiO₂, eluting with DCM/MeOH=100/1 to 20/1) providing the title compound Intermediate F2-Peg12-3 (18.5 g) as a yellow oil. LCMS Method C3: Rt=1.19 min, MS m/z [M+H]⁺=1552.8.

### Step 4: 15-((39-((2,5-Dioxopyrrolidin-1-yl)oxy)-39-oxo-3,6,9,12,15,18,21,24,27,30,33,36-dodecaoxanonatriacontyl)carbamoyl)nonacosane-1,15,29-tricarboxylic acid (Intermediate F2-Peg12)

To a solution of 41,55-dibenzyl 1-(2,5-dioxopyrrolidin-1-yl) 41-(15-(benzyloxy)-15-oxopentadecyl)-40-oxo-3,6,9,12,15,18,21,24,27,30,33,36-dodecaoxa-39-azapentapentacontane-1,41,55-tricarboxylate (Intermediate F2-Peg12-3, 18.5 g, 11.9 mmol) in THF (150 mL) was added Pd/C (1.27 g, 1.19 mmol, 10 wt. %) and Pd(OH)₂/C (838 mg, 1.19 mmol, 20 wt. %) under a N₂ atmosphere. The atmosphere was replaced with H₂ from a balloon reservoir and the mixture was stirred at 25° C. for 12 h. The RM was filtered and concentrated under reduced pressure. The resulting residue was purified by column chromatography (SiO₂, eluting with DCM/MeOH=1/0 to 20/1) providing the title compound Intermediate F2-Peg12 (11.3 g) as a white gum. ¹H NMR (400 MHz, Chloroform-d) δ [ppm] 3.86 (t, J=6.6 Hz, 2H), 3.61-3.71 (m, 44H), 3.55-3.61 (m, 2H), 3.49-3.55 (m, 2H), 2.91 (t, J=6.6 Hz, 2H), 2.81-2.88 (m, 4H), 2.34 (t, J=7.6 Hz, 4H), 1.96-2.07 (m, 2H), 1.69-1.80 (m, 2H), 1.63 (m, 4H), 1.19-1.38 (m, 44H). LCMS Method C3: Rt=0.95 min, MS m/z [M+H]⁺=1281.7.

### Compound Examples

### Method 1

### Example A1: 50-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,47,52-tetraoxo-51-undecyl-3,10,13,16,19,22,25,28,31,34,37,40,43-tridecaoxa-6,46,51-triazadohexacontan-62-oic acid

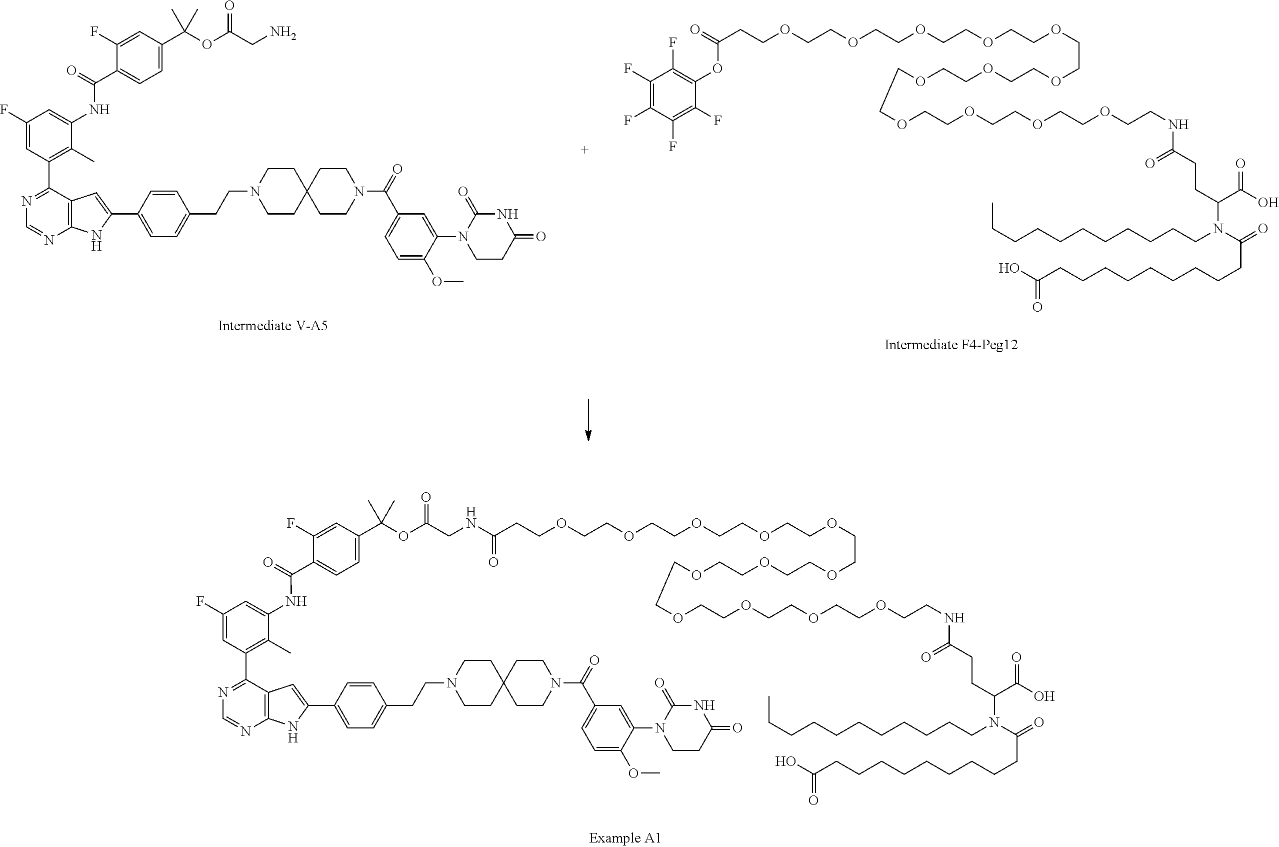 [SMILES: COc1ccc(C(=O)N2CCC3(CCN(CCc4ccc(-c5cc6c(-c7cc(F)cc(NC(=O)c8ccc(C(C)(C)OC(=O)CN)cc8F)c7C)ncnc6[nH]5)cc4)CC3)CC2)cc1N1CCC(=O)NC1=O ; CCCCCCCCCCCN(C(=O)CCCCCCCCCC(=O)O)C(CCC(=O)NCCOCCOCCOCCOCCOCCOCCOCCOCCOCCOCCOCCOCCC(=O)Oc1c(F)c(F)c(F)c(F)c1F)C(=O)O ; CCCCCCCCCCCN(C(=O)CCCCCCCCCC(=O)O)C(CCC(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

A stirring solution of 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl glycinate (Intermediate V-A5, 2.242 g) in anhydrous DMF (20 mL) was treated with a solution of 44-carboxy-1,41,46-trioxo-1-(perfluorophenoxy)-45-undecyl-4,7,10,13,16,19,22,25,28,31,34,37-dodecaoxa-40,45-diazahexapentacontan-56-oic acid (Intermediate F4-Peg12, 1.676 g, 1.192 mmol) in anhydrous DMF (20 mL) and then DIPEA (2.313 mL, 13.24 mmol) was added. The RM was stirred at RT for 1 h and then ACN (150 mL) was added. The heterogeneous mixture was ultrasonicated for 5 min and then filtered to remove the solids. The filter cake washed with ACN (50 mL) and then dried under reduced pressure. Purification by chromatography on a C18 column (RediSep® Gold, 275 g) eluting with ACN/water (0.1% formic acid as modifier) from 10-100% afforded, after lyophilization, the title compound Example A1 (0.74 g) as a white, fluffy powder. LCMS Method W7: Rt=6.16 min; MS m/z [M−2H]²⁻=1031.1. LCMS Method CB-A: Rt=23.33 min; ee 3.1%.

### Example A2: (METHOD 2) (S)-50-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,47,52-tetraoxo-51-undecyl-3,10,13,16,19,22,25,28,31,34,37,40,43-tridecaoxa-6,46,51-triazadohexacontan-62-oic acid

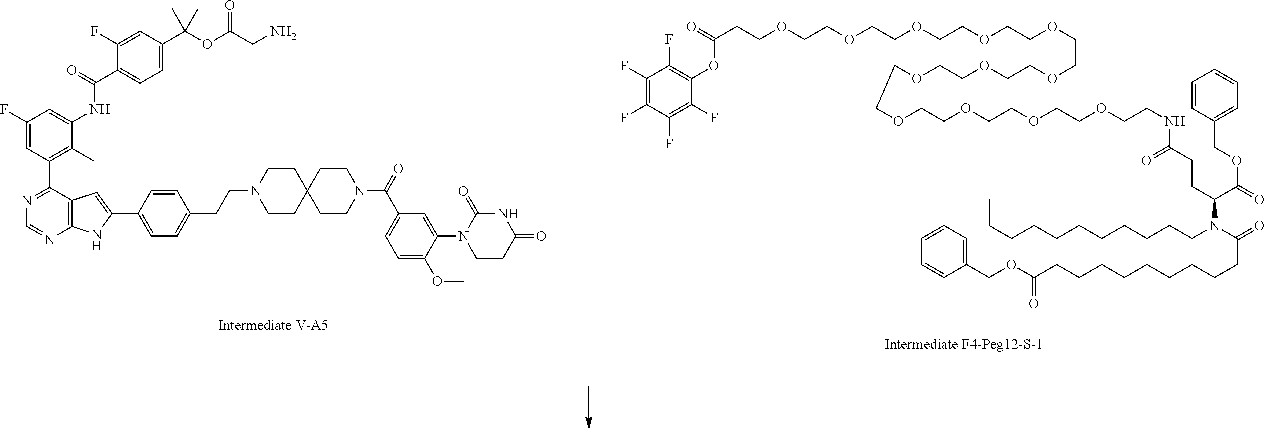 [SMILES: COc1ccc(C(=O)N2CCC3(CCN(CCc4ccc(-c5cc6c(-c7cc(F)cc(NC(=O)c8ccc(C(C)(C)OC(=O)CN)cc8F)c7C)ncnc6[nH]5)cc4)CC3)CC2)cc1N1CCC(=O)NC1=O ; CCCCCCCCCCCN(C(=O)CCCCCCCCCC(=O)OCc1ccccc1)[C@@H](CCC(=O)NCCOCCOCCOCCOCCOCCOCCOCCOCCOCCOCCOCCOCCC(=O)Oc1c(F)c(F)c(F)c(F)c1F)C(=O)OCc1ccccc1]

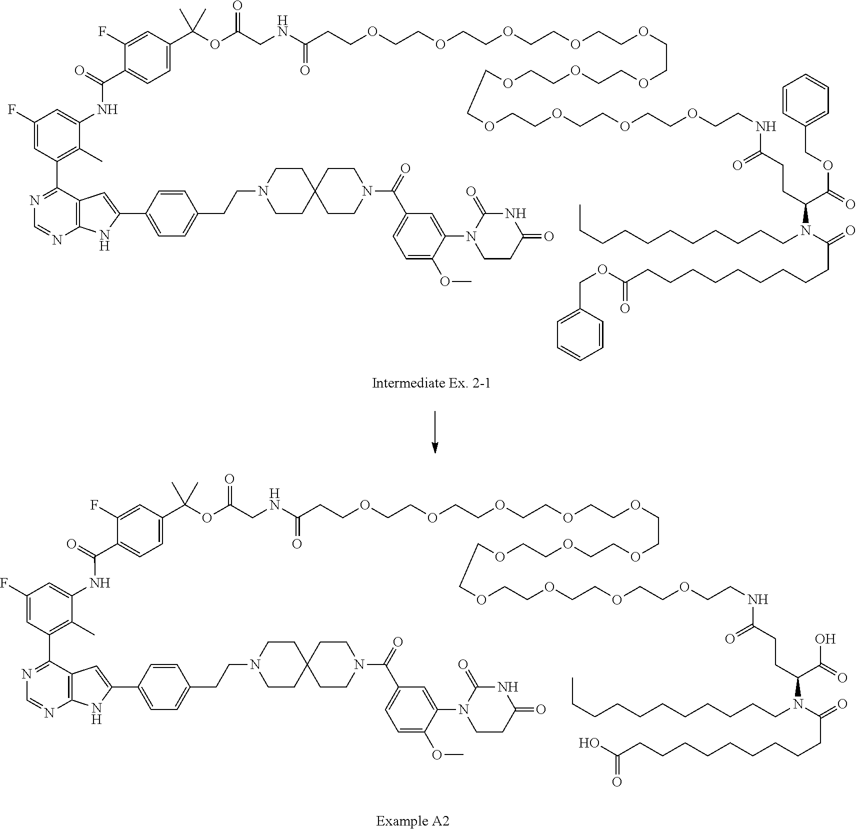 [SMILES: CCCCCCCCCCCN(C(=O)CCCCCCCCCC(=O)OCc1ccccc1)[C@@H](CCC(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)OCc1ccccc1 ; CCCCCCCCCCCN(C(=O)CCCCCCCCCC(=O)O)[C@@H](CCC(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

### Step 1: 46,57-Dibenzyl 1-(2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl) (S)-3,43,48-trioxo-47-undecyl-6,9,12,15,18,21,24,27,30,33,36,39-dodecaoxa-2,42,47-triazaheptapentacontane-1,46,57-tricarboxylate (Intermediate Ex. 2-1)

A stirring solution of Intermediate V-A5 (6.9 g, 5.21 mmol) and DIPEA (4.55 mL, 26.1 mmol) in DMF (70 mL) was cooled to 0° C. A solution of 43,54-dibenzyl 1-(perfluorophenyl) (S)-40,45-dioxo-44-undecyl-3,6,9,12,15,18,21,24,27,30,33,36-dodecaoxa-39,44-diazatetrapentacontane-1,43,54-tricarboxylate (Intermediate F4-Peg12-S-1, 10.36 g, 5.73 mmol) in DMF (20 mL) was added and the RM was allowed to warm to RT. Stirring was continued for 1 h at RT and then concentrated under reduced pressure. Purification by chromatography over silica gel (RediSep® Rf, 120 g) eluting with MeOH in DCM (0 to 5%) gave the title compound Intermediate Ex. 2-1 (11 g). LCMS Method W5: Rt=1.37 min; MS m/z [M+2H]²⁺=1122.1.

### Step 2: (S)-50-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,47,52-tetraoxo-51-undecyl-3,10,13,16,19,22,25,28,31,34,37,40,43-tridecaoxa-6,46,51-triazadohexacontan-62-oic acid (Example A2)

To a solution of 46,57-dibenzyl 1-(2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl) (S)-3,43,48-trioxo-47-undecyl-6,9,12,15,18,21,24,27,30,33,36,39-dodecaoxa-2,42,47-triazaheptapentacontane-1,46,57-tricarboxylate (Intermediate Ex. 1-1, 11 g) in MeOH/THF (4/1, 100 mL) was added Pd/C (BASF 4505 D/R E 1.878 g, 1.765 mmol, 10 wt. %) and the RM agitated under ˜0.1 bar H₂ atmosphere for 30 h at RT. The RM was filtered twice over Celite® and then the filtrate passed sequentially through three Agilent Stratospheres PL-Thiol MP cartridges (3×5 g). The cartridges were washed with MeOH/THF (3/1, 250 mL) and the filtrate pre-adsorbed onto Isolute® H-MN. The mixture was concentrated under reduced pressure and purified by chromatography on C18 (RediSep® Gold, 415 g) eluting with ACN/water (0.1% formic acid as modifier) from 5-100%. Lyophilization of the obtained product gave the title compound Example A2 as a white, fluffy powder (6 g). LCMS Method W1: Rt=6.03 min; m/z [M−2H]²⁻=1030.2; LCMS Method CB-A: Rt=22.32 min; ee >98%. The obtained product (0.05 g, 0.024 mmol) was dissolved in ACN/water (2/1, 3 mL) and 1N aq. NaOH (0.048 mL, 0.048 mmol) added. The solution was stirred at RT for 1 h and then lyophilized to give the Na salt of the title compound Example A2 as a white fluffy powder (0.052 g). LCMS Method W11: Rt=1.15 min; m/z [M+2H]²⁺=1032.1.

The following examples were prepared, using the appropriate starting materials, according to the procedures described for the Example A1 and Example A2 herein above.

### Example A3: (R)-50-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,47,52-tetraoxo-51-undecyl-3,10,13,16,19,22,25,28,31,34,37,40,43-tridecaoxa-6,46,51-triazadohexacontan-62-oic acid

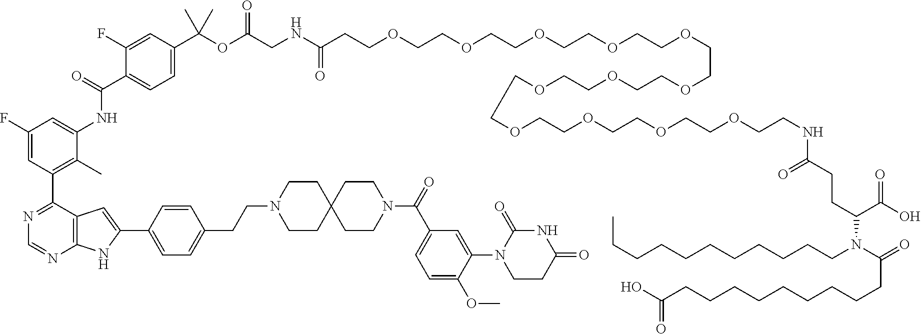

Example A3 was prepared according to the method described in Example A1 herein above using Intermediate V-A5 and freshly prepared Intermediate F4-Peg12-R. LCMS Method W1: Rt=5.95 min; m/z [M−2H]²⁻=1030.0; LCMS Method CB-A: Rt=34.23 min; ee 91.4%.

### Example A4: 51-Carboxy-2-(4-((3-(6-(4-(2-(9-(3-(2,4-dioxotetrahydropyrimidin-(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2,5,5-trimethyl-4,8,48,53-tetraoxo-52-undecyl-3,11,14,17,20,23,26,29,32,35,38,41,44-tridecaoxa-7,47,52-triazatrihexacontan-63-oic acid

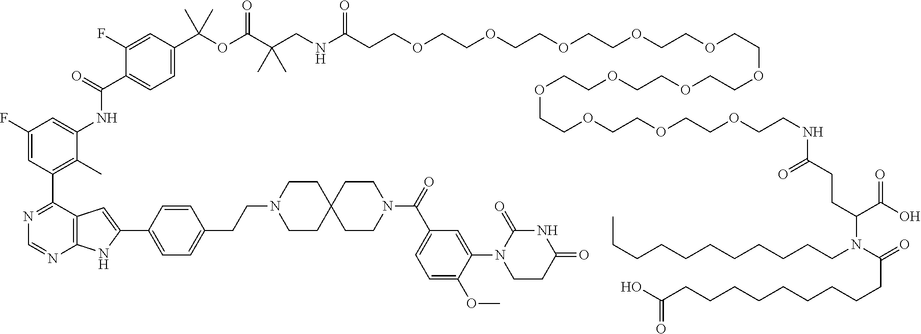 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCC(C)(C)C(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A4 was prepared according to the method described in Example A1 herein above using Intermediate V-A3 and Intermediate F4-Peg12. LCMS Method T8: Rt=2.52 min; MS m/z [M+2H]²⁺=1053.0.

### Example A5: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-55-(11-hydroxy-N-undecylundecanamido)-2-methyl-4,8,12,52-tetraoxo-3,15,18,21,24,27,30,33,36,39,42,45,48-tridecaoxa-7,11,51-triazahexapentacontan-56-oic acid

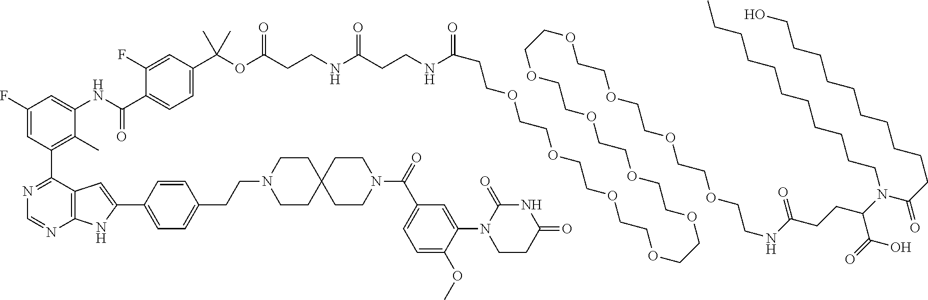 [SMILES: CCCCCCCCCCCN(C(=O)CCCCCCCCCCO)C(CCC(=O)NCCOCCOCCOCCOCCOCCOCCOCCOCCOCCOCCOCCOCCC(=O)NCCC(=O)NCCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A5 was prepared according to the method described in Example A1 herein above, using Intermediate V-A4 and Intermediate F4-Peg12. LCMS Method T10: Rt=1.79 min; MS m/z [M+2H]²⁺=1075.4.

### Example A6: (S)-11-((1-Carboxy-4-((2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)amino)-4-oxobutyl)(undecyl)amino)-11-oxoundecanoic acid

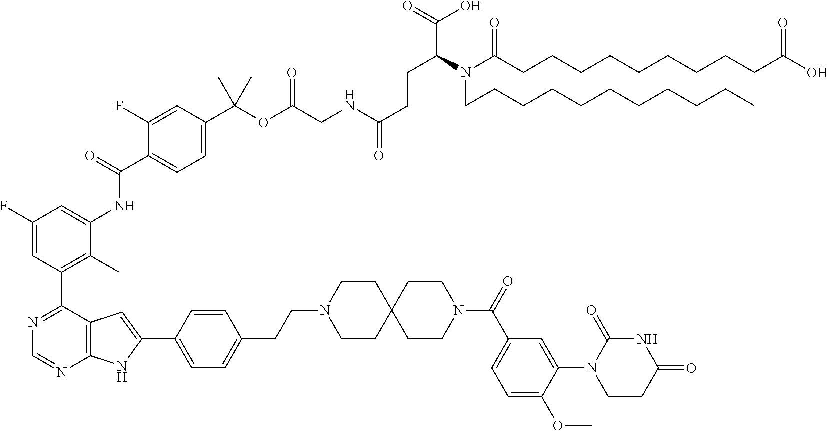 [SMILES: CCCCCCCCCCCN(C(=O)CCCCCCCCCC(=O)O)[C@@H](CCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A6 was prepared according to the method described in Example A2 herein above using Intermediate V-A5 and Intermediate F4-S. Step 1: LCMS Method W6: Rt=1.53 min; MS m/z [M−H]⁻=1643.0. Step 2: LCMS Method W7: Rt=6.49 min; MS m/z [M−H]⁻=1462.1.

### Example A7: 26-Carboxy-2-(4-((3-(6-(4-(2-(9-(3-(2,4-dioxotetrahydropyrimidin-(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,23,28-tetraoxo-27-undecyl-3,10,13,16,19-pentaoxa-6,22,27-triazaoctatriacontan-38-oicacid

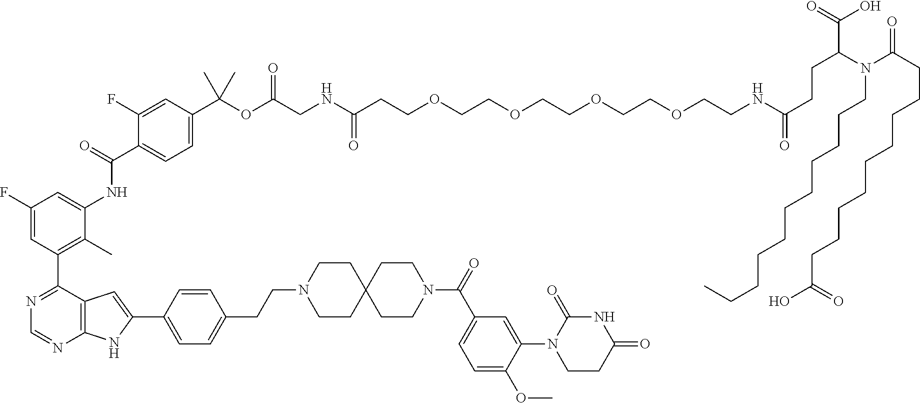 [SMILES: CCCCCCCCCCCN(C(=O)CCCCCCCCCC(=O)O)C(CCC(=O)NCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A7 was prepared according to the method described in Example A1 herein above using Intermediate V-A5 HCl salt and Intermediate FA4-Peg4. LCMS Method W7: Rt=6.12 min; MS m/z [M−2H]²⁻=853.8.

### Example A8: 38-Carboxy-2-(4-((3-(6-(4-(2-(9-(3-(2,4-dioxotetrahydropyrimidin-(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,35,40-tetraoxo-39-undecyl-3,10,13,16,19,22,25,28,31-nonaoxa-6,34,39-triazapentacontan-50-oic acid

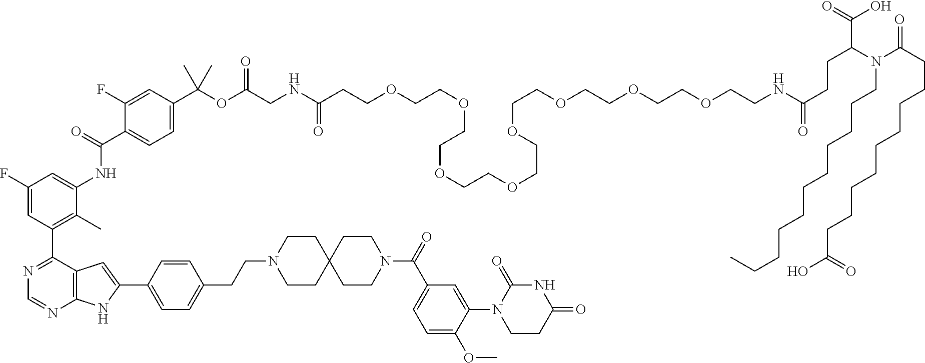 [SMILES: CCCCCCCCCCCN(C(=O)CCCCCCCCCC(=O)O)C(CCC(=O)NCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A8 was prepared according to the method described in Example A1 herein above using Intermediate V-A5 and Intermediate FA4-Peg8. LCMS Method W7: Rt=6.27 min; MS m/z [M−2H]²⁻=942.0. The sodium salt of Example A8 was prepared according to the method described for the preparation of the sodium salt of Example A13 herein below.

### Example A9: 62-Carboxy-2-(4-((3-(6-(4-(2-(9-(3-(2,4-dioxotetrahydropyrimidin-(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,59,64-tetraoxo-63-undecyl-3,10,13,16,19,22,25,28,31,34,37,40,43,46,49,52,55-heptadecaoxa-6,58,63-triazatetraheptacontan-74-oic acid

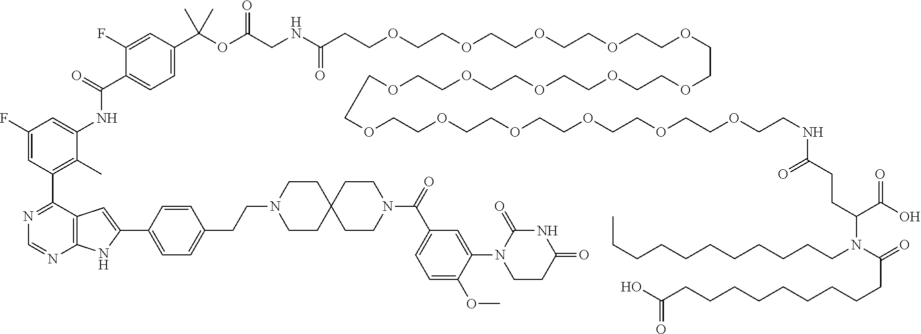 [SMILES: CCCCCCCCCCCN(C(=O)CCCCCCCCCC(=O)O)C(CCC(=O)NCCOCCOCCOCCOCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A9 was prepared according to the method described in Example A1 herein above using Intermediate V-A5 and Intermediate F4-Peg16. LCMS Method T7: Rt=1.31 min; MS m/z [M+2H]²⁺=1121.3.

### Example A10: 2-((44-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-44-methyl-39,42-dioxo-3,6,9,12,15,18,21,24,27,30,33,36,43-tridecaoxa-40-azapentatetracontyl)carbamoyl)-2-undecyltridecanedioic acid

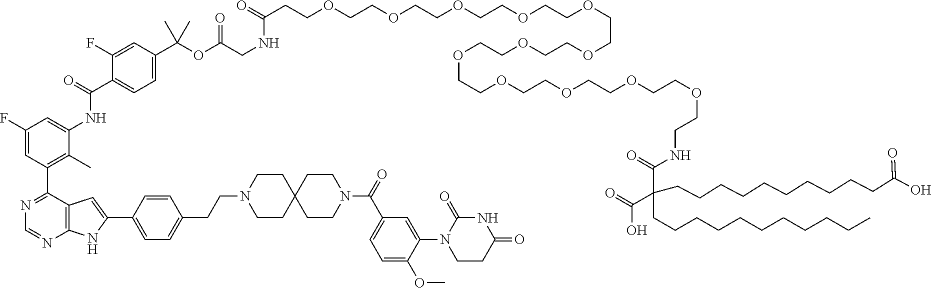 [SMILES: CCCCCCCCCCCC(CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1]

To a solution of 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (1-amino-3,6,9,12,15,18,21,24,27,30,33,36-dodecaoxanonatriacontan-39-oyl)glycinate TFA salt (Intermediate V-A5-Peg12, 235 mg) and DIPEA (0.36 mL) in DCM (2 mL) was added 2-(((2,5-dioxopyrrolidin-1-yl)oxy)carbonyl)-2-undecyltridecanedioic acid (Intermediate rac-F1-3, 80 mg, 0.149 mmol) and the RM was allowed to stir for 1 h. The volatile solvent was removed under a stream of N₂ and the residue was dried under high vacuum for ˜2 h. The sample was diluted with DMSO (2 mL) and purified over a RP Interchim Puriflash PT C4 column (50 g, spherical silica, 15 μm, 200 Å) eluting with 5% ACN/water (0.1% formic acid as modifier) for 5 min followed by 5 to 50% ACN/water (0.1% formic acid) to provide, after lyophilization, the title compound Example A10 (210 mg). LCMS Method T8: Rt=3.00 min; MS m/z [M+2H]²⁺=1004.0.

### Example A11: 2-((80-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-80-methyl-75,78-dioxo-3,6,9,12,15,18,21,24,27,30,33,36,39,42,45,48,51,54,57,60,63,66,69,72,79-pentacosaoxa-76-azahenoctacontyl)carbamoyl)-2-undecyltridecanedioic acid

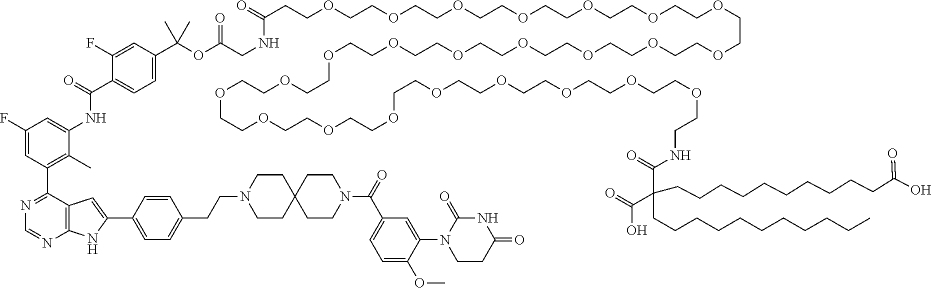 [SMILES: CCCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1]

Example A11 was prepared according to the method described in Example A1, above using Intermediate V-A5-Peg24 and Intermediate rac-F1-3. The sodium salt of Example A11 was prepared according to the method described for the preparation of the sodium salt of Example A13 herein below. LCMS Method T8: Rt=3.13 min; MS m/z [M+2H]²⁺=1268.3.

### Example A12: 51-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,8,48,53-tetraoxo-52-undecyl-3,11,14,17,20,23,26,29,32,35,38,41,44-tridecaoxa-7,47,52-triazatrihexacontan-63-oic acid

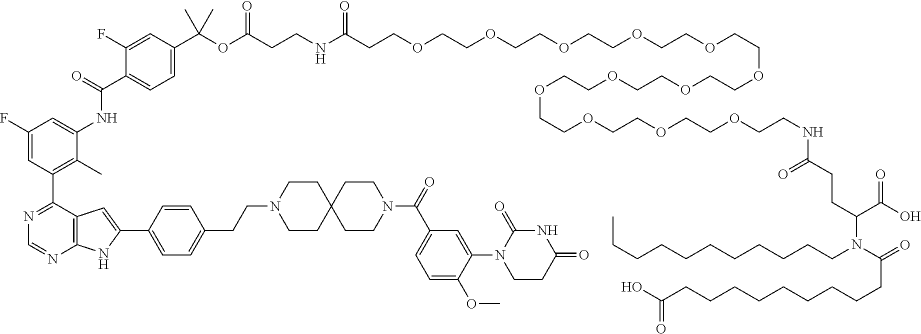 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A12 was prepared according to the method described in Example A1 herein above using Intermediate V-A1 and Intermediate F4-Peg12. LCMS Method T7: Rt=1.08 min; MS m/z [M+2H]²⁺=1040.2.

### Example A13: 50-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-6-ethyl-2-methyl-4,7,47,52-tetraoxo-51-undecyl-3,10,13,16,19,22,25,28,31,34,37,40,43-tridecaoxa-6,46,51-triazadohexacontan-62-oic acid

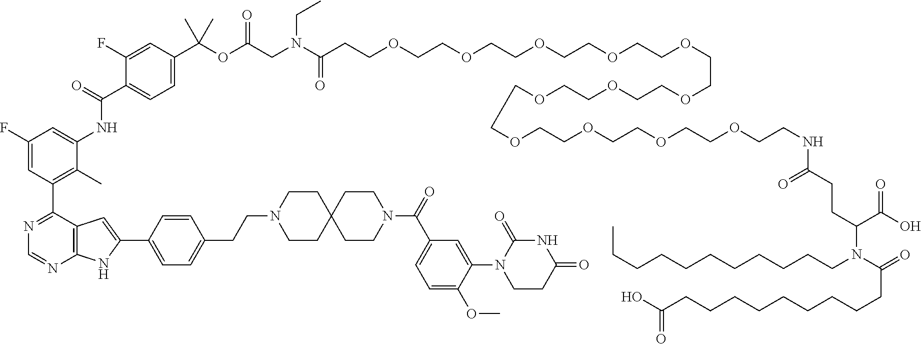 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)N(CC)CC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A13 was prepared according to the method described in Example A1, above, using Intermediate V-A2 and Intermediate F4-Peg12. The sodium salt of Example A13 was prepared as described herein below. LCMS Method W7: Rt=6.41 min; MS m/z [M+2H]²⁺=1046.6.

### Method for the Conversion of Example A13 to the Sodium Salt

To a solution of 50-carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-6-ethyl-2-methyl-4,7,47,52-tetraoxo-51-undecyl-3,10,13,16,19,22,25,28,31,34,37,40,43-tridecaoxa-6,46,51-triazadohexacontan-62-oic acid (Example A13, 500 mg, 0.239 mmol) in ACN (30 mL) and water (10 mL) was added 1N aq. NaOH solution (0.478 mL, 0.478 mmol, 2 eq.). The solution was shaken for 1 h at RT and then lyophilized to provide the Na salt of (470 mg) of Example A13. LCMS Method W7: Rt=6.39 min; MS m/z=1046.7 [M+2H]²⁺.

### Example A14: 46-Carboxy-1-(4-(((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)carbonyl)phenyl)-3,43,48-trioxo-47-undecyl-6,9,12,15,18,21,24,27,30,33,36,39-dodecaoxa-2,42,47-triazaoctapentacontan-58-oic acid

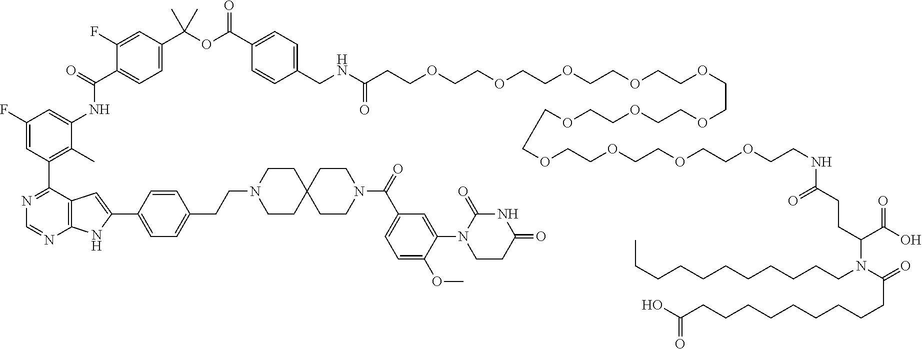 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCc1ccc(C(=O)OC(C)(C)c2ccc(C(=O)Nc3cc(F)cc(-c4ncnc5[nH]c(-c6ccc(CCN7CCC8(CC7)CCN(C(=O)c7ccc(OC)c(N9CCC(=O)NC9=O)c7)CC8)cc6)cc45)c3C)c(F)c2)cc1)C(=O)O]

Example A14 was prepared according to the method described in Example A1 herein above using Intermediate V-A14 and Intermediate F4-Peg12. LCMS Method T7: Rt=1.10 min; MS m/z [M+2H]²⁺=1070.9.

### Example A15: 44-Carboxy-1-(4-(((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)carbonyl)-4-fluoropiperidin-1-yl)-1,41,46-trioxo-45-undecyl-4,7,10,13,16,19,22,25,28,31,34,37-dodecaoxa-40,45-diazahexapentacontan-56-oic acid

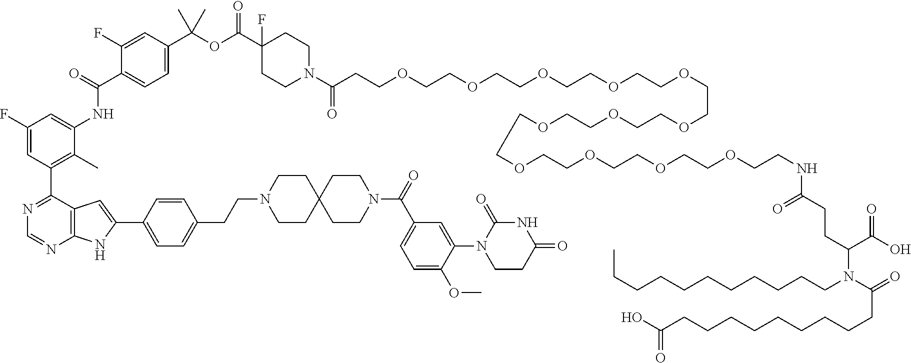 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)N1CCC(F)(C(=O)OC(C)(C)c2ccc(C(=O)Nc3cc(F)cc(-c4ncnc5[nH]c(-c6ccc(CCN7CCC8(CC7)CCN(C(=O)c7ccc(OC)c(N9CCC(=O)NC9=O)c7)CC8)cc6)cc45)c3C)c(F)c2)CC1)C(=O)O]

Example A15 was prepared according to the method described in Example A1 herein above using Intermediate V-A15 and Intermediate F4-Peg12. LCMS Method W7: Rt=6.39 min; MS m/z [M+2H]²⁺=1069.0.

### Example A16: (5S)-50-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2,5-dimethyl-4,7,47,52-tetraoxo-51-undecyl-3,10,13,16,19,22,25,28,31,34,37,40,43-tridecaoxa-6,46,51-triazadohexacontan-62-oic acid

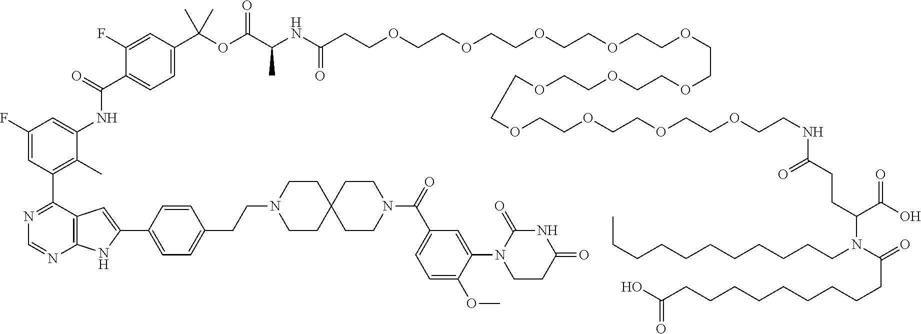 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)N[C@@H](C)C(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A16 was prepared according to the method described in Example A1 herein above, using Intermediate V-A6 and Intermediate F4-Peg12. LCMS Method T8: Rt=2.53 min; MS m/z [M+2H]²⁺=1039.6.

### Example A17: (5R)-50-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2,5-dimethyl-4,7,47,52-tetraoxo-51-undecyl-3,10,13,16,19,22,25,28,31,34,37,40,43-tridecaoxa-6,46,51-triazadohexacontan-62-oic acid

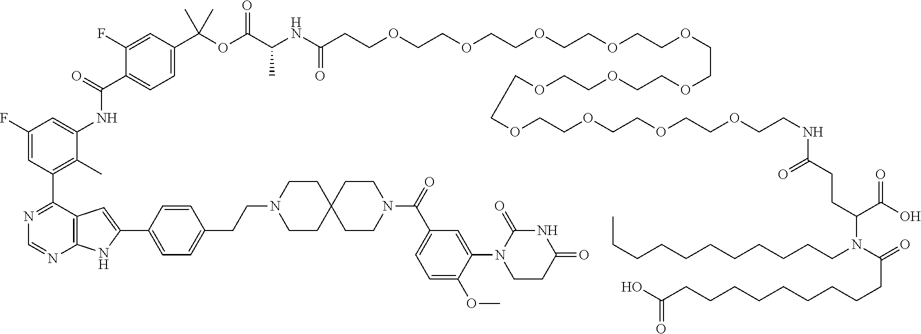 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)N[C@H](C)C(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A17 was prepared according to the method described in Example A1 herein above, using Intermediate V-A7 and Intermediate F4-Peg12. LCMS Method T8: Rt=2.53 min; MS m/z [M+2H]²⁺=1039.7.

### Example A18: 51-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2,6,6-trimethyl-4,8,48,53-tetraoxo-52-undecyl-3,11,14,17,20,23,26,29,32,35,38,41,44-tridecaoxa-7,47,52-triazatrihexacontan-63-oic acid

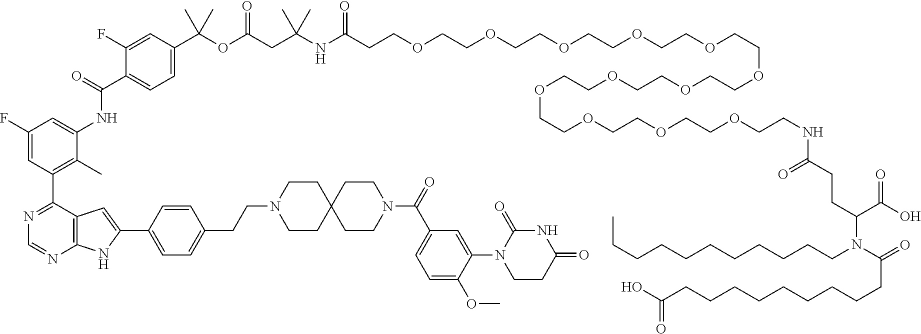 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NC(C)(C)CC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A10 was prepared according to the method described in Example A1 herein above, using Intermediate V-A8 and Intermediate F4-Peg12. LCMS Method T8: Rt=2.54 min; MS m/z [M+2H]²⁺=1053.8.

### Example A19: 44-Carboxy-1-((S)-2-(2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)pyrrolidin-1-yl)-1,41,46-trioxo-45-undecyl-4,7,10,13,16,19,22,25,28,31,34,37-dodecaoxa-40,45-diazahexapentacontan-56-oic acid

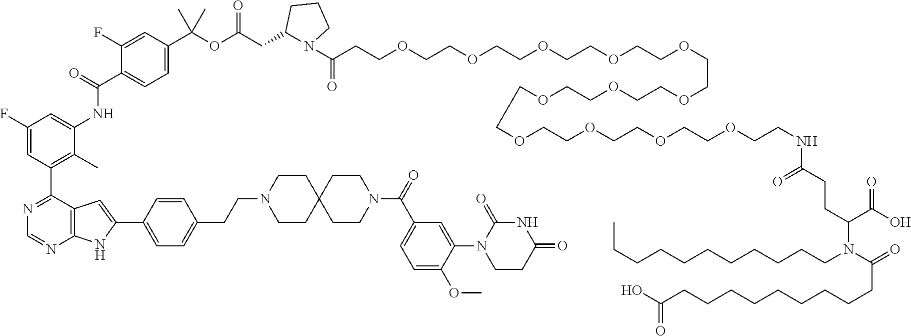

Example A19 was prepared according to the method described in Example A1 herein above, using Intermediate V-A10 and Intermediate F4-Peg12. LCMS Method T9: Rt=0.83 min; MS m/z [M+2H]²⁺=1060.0.

### Example A20: (6S)-51-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2,6-dimethyl-4,8,48,53-tetraoxo-52-undecyl-3,11,14,17,20,23,26,29,32,35,38,41,44-tridecaoxa-7,47,52-triazatrihexacontan-63-oic acid

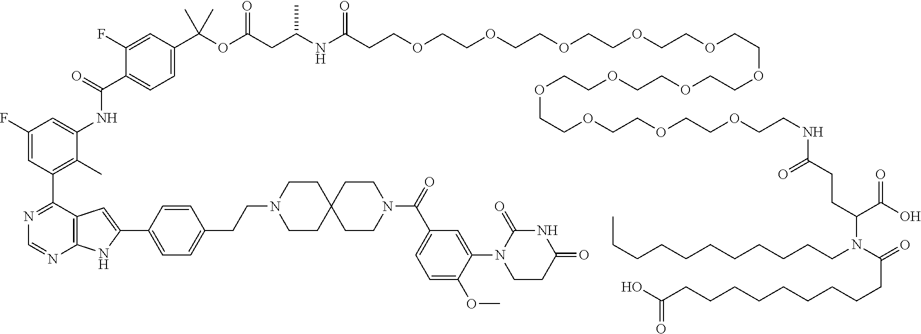 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)N[C@@H](C)CC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A20 was prepared according to the method described in Example A1 herein above using Intermediate V-A9 and Intermediate F4-Peg12. LCMS Method T9: Rt=0.77 min; MS m/z [M+2H]²⁺=1047.0.

### Example A21: 44-Carboxy-1-(4-(2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)piperidin-1-yl)-1,41,46-trioxo-45-undecyl-4,7,10,13,16,19,22,25,28,31,34,37-dodecaoxa-40,45-diazahexapentacontan-56-oic acid

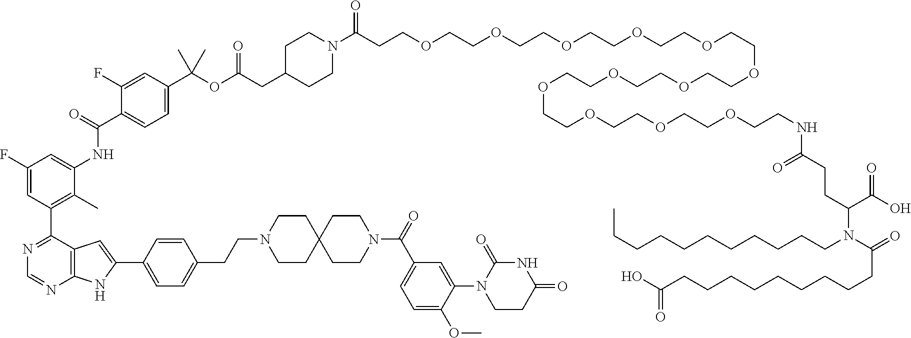 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)N1CCC(CC(=O)OC(C)(C)c2ccc(C(=O)Nc3cc(F)cc(-c4ncnc5[nH]c(-c6ccc(CCN7CCC8(CC7)CCN(C(=O)c7ccc(OC)c(N9CCC(=O)NC9=O)c7)CC8)cc6)cc45)c3C)c(F)c2)CC1)C(=O)O]

Example A21 was prepared according to the method described in Example A1 herein above using Intermediate V-A16 and Intermediate F4-Peg12. LCMS Method T7: Rt=1.09 min; MS m/z [M+2H]²⁺=1067.2.

### Example A22: 44-Carboxy-1-(4-(2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)piperazin-1-yl)-1,41,46-trioxo-45-undecyl-4,7,10,13,16,19,22,25,28,31,34,37-dodecaoxa-40,45-diazahexapentacontan-56-oic acid

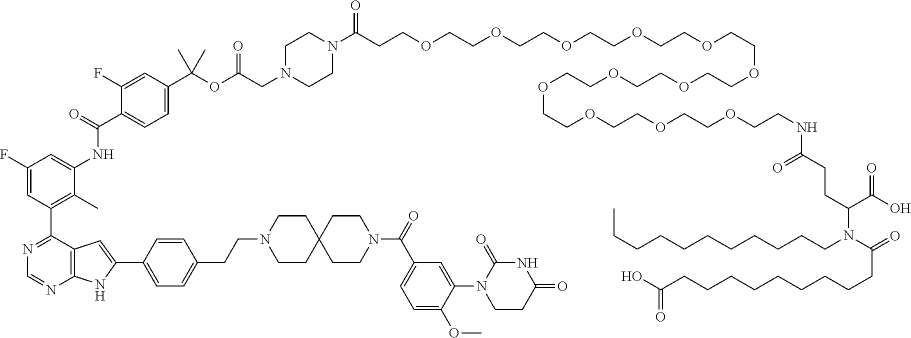 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)N1CCN(CC(=O)OC(C)(C)c2ccc(C(=O)Nc3cc(F)cc(-c4ncnc5[nH]c(-c6ccc(CCN7CCC8(CC7)CCN(C(=O)c7ccc(OC)c(N9CCC(=O)NC9=O)c7)CC8)cc6)cc45)c3C)c(F)c2)CC1)C(=O)O]

Example A22 was prepared according to the method described in Example A1 herein above using Intermediate V-A17 and Intermediate F4-Peg12. LCMS Method T2: Rt=1.18 min; MS m/z [M−2H]²⁻=1065.0. The sodium salt of Example A22 was prepared according to the method described for Example A13, above. LCMS Method W7: Rt=6.14 min; MS m/z [M−2H]²⁻=1065.1.

### Example A23: 46-Carboxy-1-(4-(2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)phenyl)-3,43,48-trioxo-47-undecyl-6,9,12,15,18,21,24,27,30,33,36,39-dodecaoxa-2,42,47-triazaoctapentacontan-58-oic acid

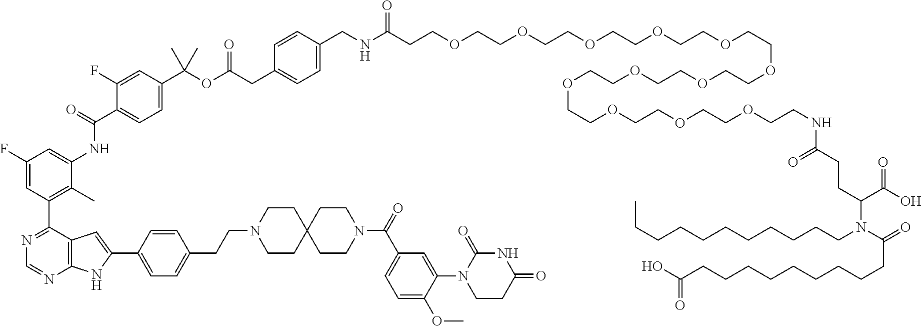 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCc1ccc(CC(=O)OC(C)(C)c2ccc(C(=O)Nc3cc(F)cc(-c4ncnc5[nH]c(-c6ccc(CCN7CCC8(CC7)CCN(C(=O)c7ccc(OC)c(N9CCC(=O)NC9=O)c7)CC8)cc6)cc45)c3C)c(F)c2)cc1)C(=O)O]

Example A23 was prepared according to the method described in Example A1 herein above, using Intermediate V-A18 and Intermediate F4-Peg12. LCMS Method T2: Rt=1.23 min; MS m/z [M−2H]²⁻=1075.4. The sodium salt of Example A23 was prepared according to the method described for the preparation of the sodium salt of Example A13 herein above LCMS Method W7: Rt=6.44 min; MS m/z [M−2H]²⁻=1075.5.

### Example A24: (S)-2-(3-((2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-3-oxopropyl)-3,43,48-trioxo-47-undecyl-6,9,12,15,18,21,24,27,30,33,36,39-dodecaoxa-2,42,47-triazaheptapentacontane-1,46,57-tricarboxylic acid

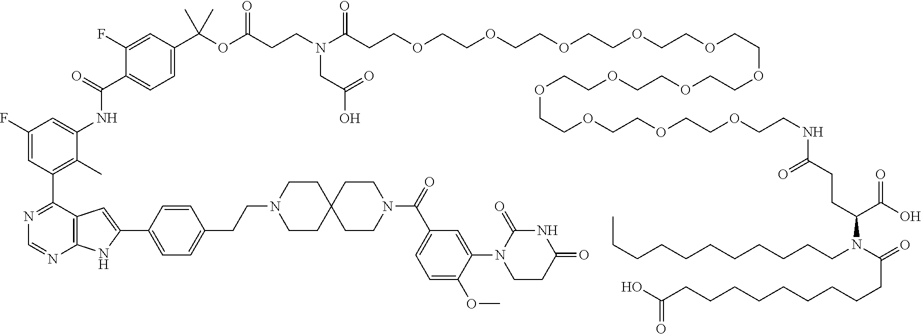 [SMILES: CCCCCCCCCCCN(C(=O)CCCCCCCCCC(=O)O)[C@@H](CCC(=O)NCCOCCOCCOCCOCCOCCOCCOCCOCCOCCOCCOCCOCCC(=O)N(CCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)CC(=O)O)C(=O)O]

Example A24 was prepared according to the method described in Example A2, above, using Intermediate V-A19 and Intermediate F4-Peg12-S-1. Step 1: LCMS Method W5: Rt=1.37 min; MS m/z [M+NH₄+2H]³+=808.1. Step 2: LCMS Method W1: Rt=6.14 min; MS m/z [M+2H]²⁺=1068.4. The sodium salt of Example A24 was prepared according to the method described for the preparation of the sodium salt of Example A13 herein above using 3 eq. of NaOH. LCMS Method W5: Rt=1.13 min; MS m/z [M−2H]²⁻=1066.2.

### Example A25: 46-Carboxy-1-(5-(((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)carbonyl)pyridin-2-yl)-3,43,48-trioxo-47-undecyl-6,9,12,15,18,21,24,27,30,33,36,39-dodecaoxa-2,42,47-triazaoctapentacontan-58-oic acid

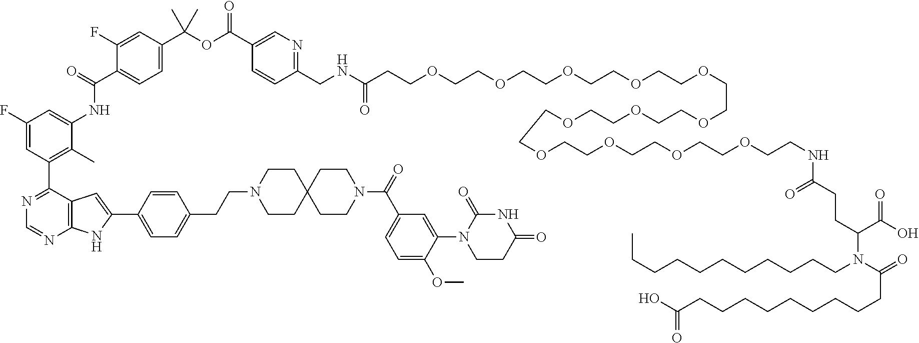 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCc1ccc(C(=O)OC(C)(C)c2ccc(C(=O)Nc3cc(F)cc(-c4ncnc5[nH]c(-c6ccc(CCN7CCC8(CC7)CCN(C(=O)c7ccc(OC)c(N9CCC(=O)NC9=O)c7)CC8)cc6)cc45)c3C)c(F)c2)cn1)C(=O)O]

Example A25 was prepared according to the method described in Example A1 herein above using Intermediate V-A12 and Intermediate F4-Peg12. LCMS Method T8: Rt=2.62 min; MS m/z [M+2H]²⁺=1071.2. The sodium salt of Example A25 was prepared according to the method described for the preparation of the sodium salt of Example A13 herein above.

### Example A26: 46-Carboxy-1-(4-(((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)carbonyl)pyridin-2-yl)-3,43,48-trioxo-47-undecyl-6,9,12,15,18,21,24,27,30,33,36,39-dodecaoxa-2,42,47-triazaoctapentacontan-58-oic acid

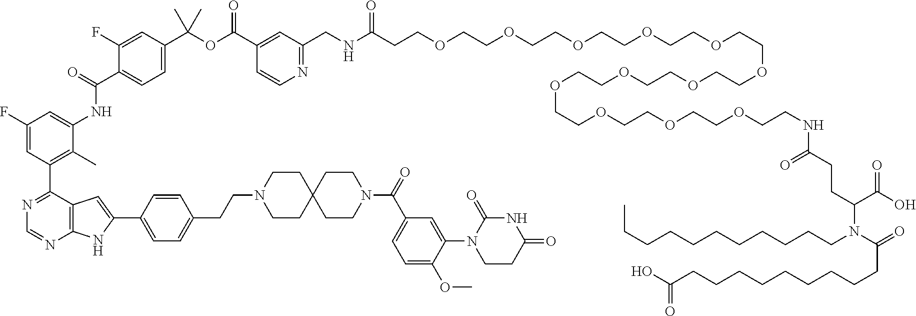 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCc1cc(C(=O)OC(C)(C)c2ccc(C(=O)Nc3cc(F)cc(-c4ncnc5[nH]c(-c6ccc(CCN7CCC8(CC7)CCN(C(=O)c7ccc(OC)c(N9CCC(=O)NC9=O)c7)CC8)cc6)cc45)c3C)c(F)c2)ccn1)C(=O)O]

Example A26 was prepared according to the method described in Example A1 herein above using Intermediate V-A13 and Intermediate F4-Peg12. LCMS Method T8: Rt=2.52 min; MS m/z [M+2H]²⁺=1071.2. The sodium salt of Example A26 was prepared according to the method described for the preparation of the sodium salt of Example A13 herein above.

### Example A27: (S)-52-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,9,49,54-tetraoxo-53-undecyl-3,12,15,18,21,24,27,30,33,36,39,42,45-tridecaoxa-8,48,53-triazatetrahexacontan-64-oic acid

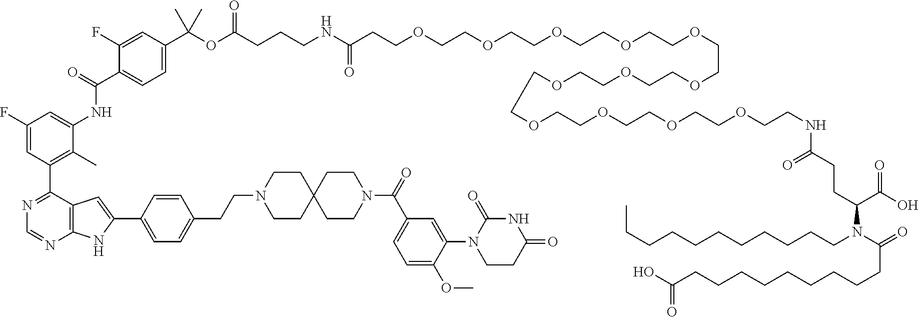 [SMILES: CCCCCCCCCCCN(C(=O)CCCCCCCCCC(=O)O)[C@@H](CCC(=O)NCCOCCOCCOCCOCCOCCOCCOCCOCCOCCOCCOCCOCCC(=O)NCCCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

Example A27 was prepared according to the method described in Example A2 herein above Intermediate V-A11 and Intermediate F4-Peg12-S-1 and Pd(OH)₂/C (20 wt. %, wet Degussa type).

Step 1: LCMS Method T8: Rt=3.21 min; MS m/z [M+2H]²⁺=1137.0.

Step 2: LCMS Method T8: Rt=2.63 min; MS m/z [M+2H]²⁺=1046.7.

### Example A28: 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,47-trioxo-48,48-bis(14-phosphonotetradecyl)-3,10,13,16,19,22,25,28,31,34,37,40,43-tridecaoxa-6,46-diazanonatetracontan-49-oic acid

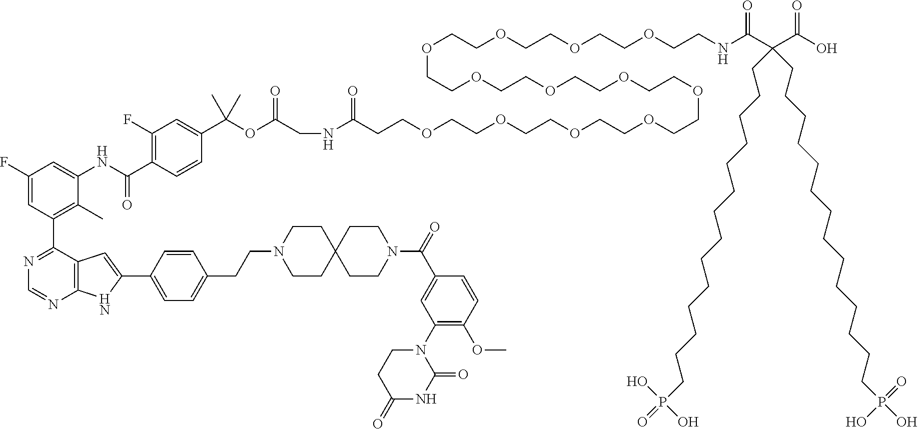 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCNC(=O)C(CCCCCCCCCCCCCCP(=O)(O)O)(CCCCCCCCCCCCCCP(=O)(O)O)C(=O)O)cc8F)c7C)ncnc6[nH]5)cc4)CC3)CC2)cc1N1CCC(=O)NC1=O]

Example A28 was prepared according to the method described in Example A1 herein above using Intermediate V-A5 and Intermediate F3-Peg12. LCMS Method T9: Rt=0.70 min; MS m/z [M+2H]²⁺=1111.7. The sodium salt of Example A28 was prepared according to the method described for the preparation of the sodium salt of Example A13 herein above.

### Example A29: 15-((44-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-44-methyl-39,42-dioxo-3,6,9,12,15,18,21,24,27,30,33,36,43-tridecaoxa-40-azapentatetracontyl)carbamoyl)nonacosane-1,15,29-tricarboxylic acid

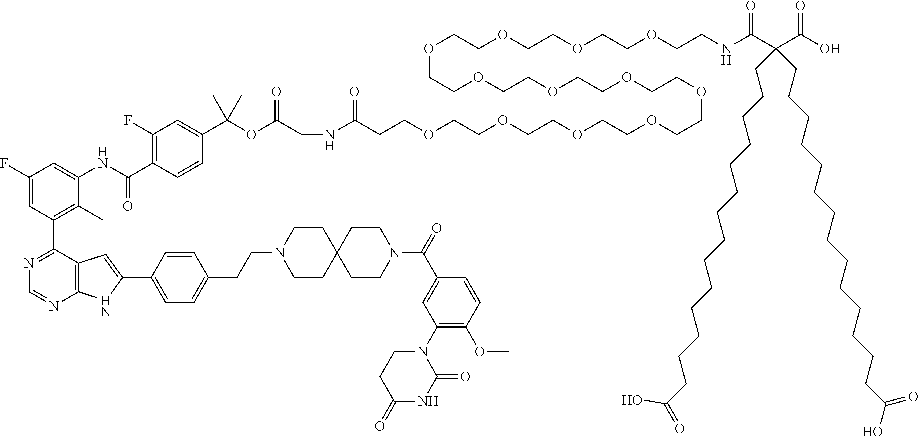 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCNC(=O)C(CCCCCCCCCCCCCCC(=O)O)(CCCCCCCCCCCCCCC(=O)O)C(=O)O)cc8F)c7C)ncnc6[nH]5)cc4)CC3)CC2)cc1N1CCC(=O)NC1=O]

Example A29 was prepared according to the method described in Example A1 herein above using Intermediate V-A5 and Intermediate F2-Peg12. LCMS Method T8: LC/MS Rt=2.85 min; MS m/z [M+2H]²⁺=1075.6. The sodium salt of Example A29 was prepared according to the method described for the preparation of the sodium salt of Example A13 herein above.

### Example A30: 23-Carboxy-1-(((3R,4S)-1-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-4-isobutylpyrrolidin-3-yl)oxy)-1,4,20,25-tetraoxo-24-undecyl-7,10,13,16-tetraoxa-3,19,24-triazapentatriacontan-35-oic acid

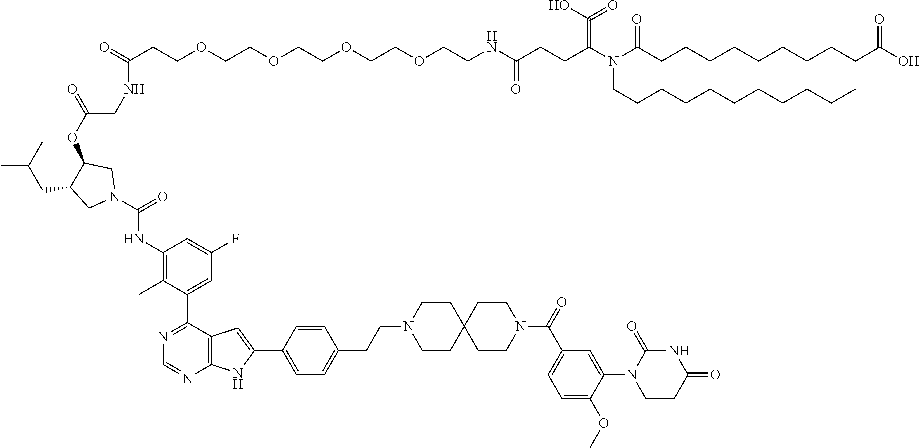

Example A30 was prepared according to the method described in Example A1 herein above using Intermediate H-A1 and Intermediate F4-Peg4. LCMS Method W7: Rt 6.06 min; MS m/z [M+2H]²⁺=851.0

### Example A31: 11-(((S)-1-Carboxy-4-((2-(((3R,4S)-1-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-4-isobutylpyrrolidin-3-yl)oxy)-2-oxoethyl)amino)-4-oxobutyl)(undecyl)amino)-11-oxoundecanoic acid

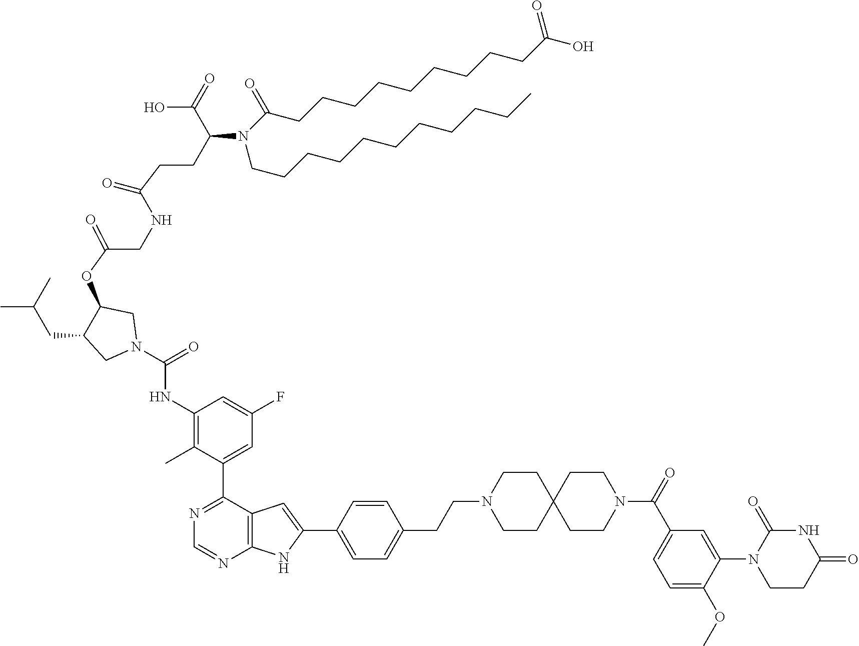 [SMILES: CCCCCCCCCCCN(C(=O)CCCCCCCCCC(=O)O)[C@@H](CCC(=O)NCC(=O)O[C@H]1CN(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)C[C@@H]1CC(C)C)C(=O)O]

Example A31 was prepared according to the method described in Example A2 herein above using Intermediate H-A1 and Intermediate F4-S. Step 1: LCMS Method T2: Rt 1.50 min; MS m/z [M+2H]²⁺=817.4. Step 2: LCMS Method W7: Rt 6.28 min; MS m/z [M+2H]²⁺=727.3.

### Example A32: 86-Carboxy-2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)-2-methyl-4,7,83,88-tetraoxo-87-undecyl-3,10,13,16,19,22,25,28,31,34,37,40,43,46,49,52,55,58,61,64,67,70,73,76,79-pentacosaoxa-6,82,87-triazaoctanonacontan-98-oic acid

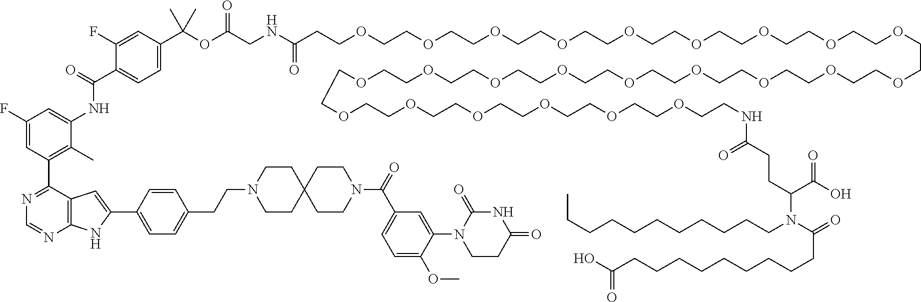 [SMILES: CCCCCCCCCCCN(C(=O)CCCCCCCCCC(=O)O)C(CCC(=O)NCCOCCOCCOCCOCCOCCOCCOCCOCCOCCOCCOCCOCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

To a solution of the TFA salt of (2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (tert-butoxycarbonyl)glycinate TFA salt (Intermediate V-A5-P, 300 mg, 0.233 mmol) in anhydrous 1,4-dioxane (7.29 mL) was added HCl (4M in 1,4-dioxane, 1.52 mL) and the RM was stirred at RT for 1.5 h. The RM was then cooled to 0° C. and DIPEA (1.426 mL, 8.16 mmol) was slowly added to the mixture. A solution of (S)-80-carboxy-1,77,82-trioxo-1-(perfluorophenoxy)-81-undecyl-4,7,10,13,16,19,22,25,28,31,34,37,40,43,46,49,52,55,58,61,64,67,70,73-tetracosaoxa-76,81-diazadononacontan-92-oic acid (Intermediate F4-Peg24, 661 mg, 0.350 mmol) in anhydrous DMF (7.29 mL) was added at 0° C. and the RM was allowed to warm up to RT. The RM was stirred at RT for 15 min, adsorbed and dried on Isolute® HMN and the residue was purified by reverse phase chromatography C18 column (RediSep® Gold, 50 g) eluting with ACN/water (0.1% formic acid as modifier) from 10 to 100% to afford after lyophilization the title compound Example A31 (82 mg) as an oil. LCMS Method W7: Rt=6.18 min; MS m/z [M+2H]²⁺=1296.6.

### Example A33: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (2-oxo-6,9,12,15,18,21,24,27,30,33,36,39-dodecaoxa-3-azadotetracontan-42-oyl)glycinate

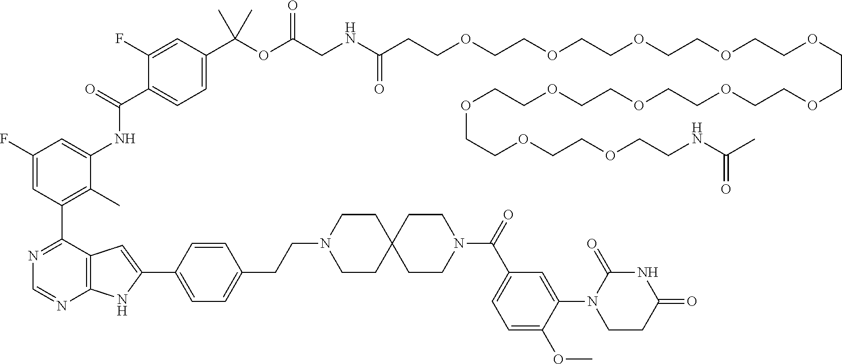 [SMILES: COc1ccc(C(=O)N2CCC3(CCN(CCc4ccc(-c5cc6c(-c7cc(F)cc(NC(=O)c8ccc(C(C)(C)OC(=O)CNC(=O)CCOCCOCCOCCOCCOCCOCCOCCOCCOCCOCCOCCOCCNC(C)=O)cc8F)c7C)ncnc6[nH]5)cc4)CC3)CC2)cc1N1CCC(=O)NC1=O]

### Step 1: Perfluorophenyl 2-oxo-6,9,12,15,18,21,24,27,30,33,36,39-dodecaoxa-3-azadotetracontan-42-oate (Intermediate Ex. 33-1)

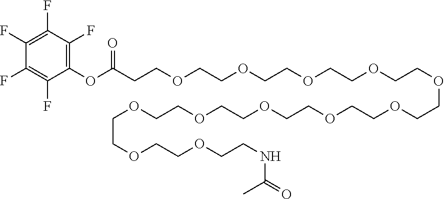 [SMILES: CC(=O)NCCOCCOCCOCCOCCOCCOCCOCCOCCOCCOCCOCCOCCC(=O)Oc1c(F)c(F)c(F)c(F)c1F]

To a solution of HOOC-dPEG12-NH₂ (CAS No. [1415408-69-3], 0.444 g, 0.711 mmol) and DIPEA (0.634 mL, 3.63 mmol) in anhydrous DCM (40 mL) was added N-acetoxysuccinimide (0.12 g, 0.73 mmol) and the RM was stirred at RT for 1 h. Bis(pentafluorophenyl)carbonate (0.324 g, 0.79 mmol) was then added and stirring was continued at RT for 2 h. The RM was concentrated under reduced pressure and then triturated ultrasonically with heptane (2×40 mL). The remaining oily residue was dried under high vacuum to give the title compound Intermediate Ex. 33-1 as an almost clear, colorless oil. (0.855 g). LCMS Method T2: (ELSD Signal) Rt=0.71 min; MS m/z [M−H−Pfp]⁻=658.5.

### Step 2: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (2-oxo-6,9,12,15,18,21,24,27,30,33,36,39-dodecaoxa-3-azadotetracontan-42-oyl)glycinate (Example A33)

To a solution of 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl glycinate (Intermediate V-A5, 0.5 g, 0.356 mmol) in anhydrous DMF (5 mL) was added a solution of perfluorophenyl 2-oxo-6,9,12,15,18,21,24,27,30,33,36,39-dodecaoxa-3-azadotetracontan-42-oate (Intermediate 33-1, 0.42 g, 0.356 mmol) in anhydrous DMF (5 mL) and then DIPEA (0.31 mL, 1.78 mmol) added. The RM was stirred at RT for 1 h and then concentrated under reduced pressure. The crude residue was triturated ultrasonically with Et₂O (2×40 mL) and the remaining solid dried under high vacuum. Purification first by chromatography over silica gel (RediSep® Rf, 40 g) eluting with MeOH in DCM (from 0 to 20%) and second over a C18 column (RediSep® Gold, 15.5 g) eluting with ACN/water with formic acid (0.1%) from 5 to 100% gave the title compound Example A33 as translucent oily solid (59 mg). LCMS Method W6: Rt=0.84 min; MS m/z [M+H]⁺=1624.1.

### Example A34: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (2,5,8,11,14,17,20,23,26,29,32,35-dodecaoxaoctatriacontan-38-oyl)glycinate

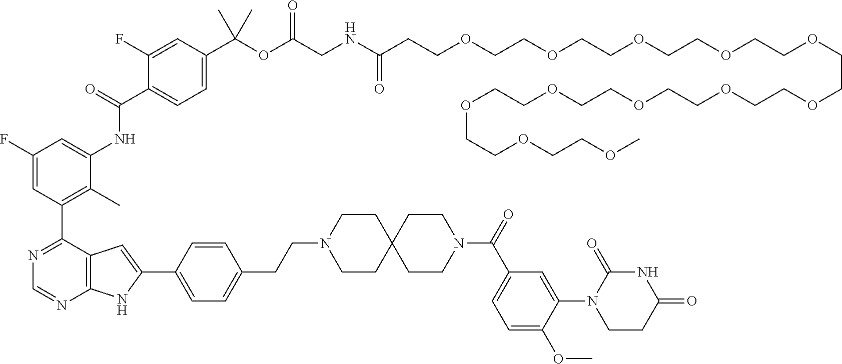 [SMILES: COCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1]

To a solution of Intermediate V-A5 HCl salt (47.1 mg, 0.046 mmol) in DCM (1 mL) was added DIPEA (1 mL, 5.73 mmol), and finally 2,5,8,11,14,17,20,23,26,29,32,35-dodecaoxaoctatriacontan-38-oic acid (57.1 mg, 0.097 mmol). HATU (36.9 mg, 0.097 mmol) was then added and the RM was stirred at RT for 3 days. Brine and DCM were added to the RM and the organic layer was washed with brine. The aq. layer was extracted with DCM and the combined organic layers were dried over MgSO₄, filtered and concentrated under reduced pressure. The resulting residue was purified on preparative HPLC X-bridge 30×50 mm 5 um column, eluting with ACN/water (0.1% formic acid as modifier) to give the title compound Example A34 (18.2 mg) as a white solid. LCMS Method T7: Rt=0.94 min; MS m/z [M+2H]²⁺=777.1.

### Example A35: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl (2,5,8,11,14,17,20,23,26,29,32,35,38,41,44,47,50,53,56,59,62,65,68,71-tetracosaoxatetraheptacontan-74-oyl)glycinate

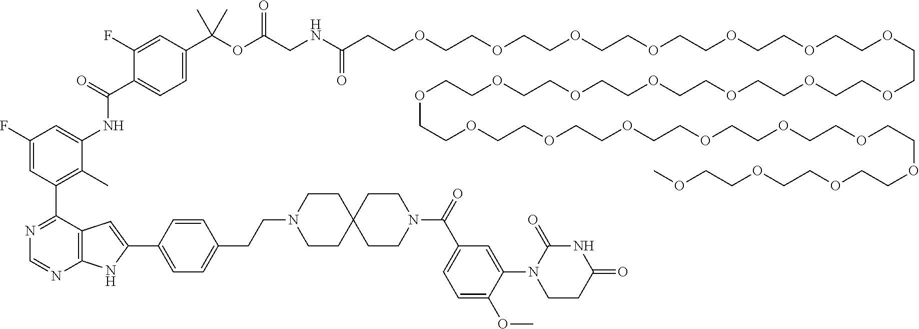 [SMILES: COCCOCCOCCOCCOCCOCCOCCOCCOCCOCCOCCOCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1]

Example A35 was prepared according to the method described herein above in Example A34 above using Intermediate V-A5 TFA salt and 2,5,8,11,14,17,20,23,26,29,33,36,39,42,45,48,51,54,57,60,63,66,69,72-tetracosaoxatetraheptacontan-74-oic acid. LCMS Method T8: Rt=1.89 min; MS m/z [M+2H]²⁺=1041.7.

### Example A36: 51-Carboxy-2-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-1-(2-fluoro-4-(2-hydroxypropan-2-yl)phenyl)-1,5,8,48,53-pentaoxo-52-undecyl-11,14,17,20,23,26,29,32,35,38,41,44-dodecaoxa-2,4,7,47,52-pentaazatrihexacontan-63-oic acid

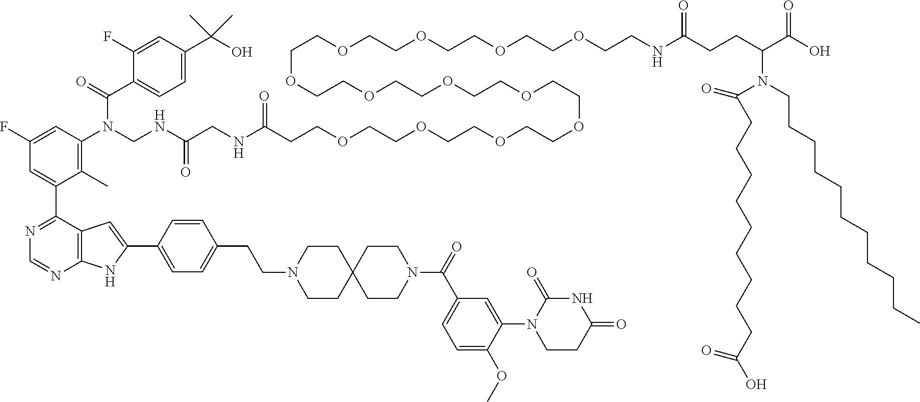 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCC(=O)NCN(C(=O)c1ccc(C(C)(C)O)cc1F)c1cc(F)cc(-c2ncnc3[nH]c(-c4ccc(CCN5CCC6(CC5)CCN(C(=O)c5ccc(OC)c(N7CCC(=O)NC7=O)c5)CC6)cc4)cc23)c1C)C(=O)O]

Example A36 was prepared according to the method described in Example A1 herein above using Intermediate M-A1 and Intermediate F4-Peg12. LCMS Method W6: Rt=1.12 min; m/z [M+2H]²⁺=1046.5, [M−2H]²⁻=1044.6. The sodium salt was prepared according to the method described for the preparation of the sodium salt of Example A13 above. LCMS Method W7: Rt=5.81 min; m/z [M−2H]²⁻=1044.6.

### Example A37: (S)-46-Carboxy-42-(2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)-38,43,48-trioxo-47-undecyl-2,5,8,11,14,17,20,23,26,29,32,35-dodecaoxa-39,42,47-triazaoctapentacontan-58-oic acid

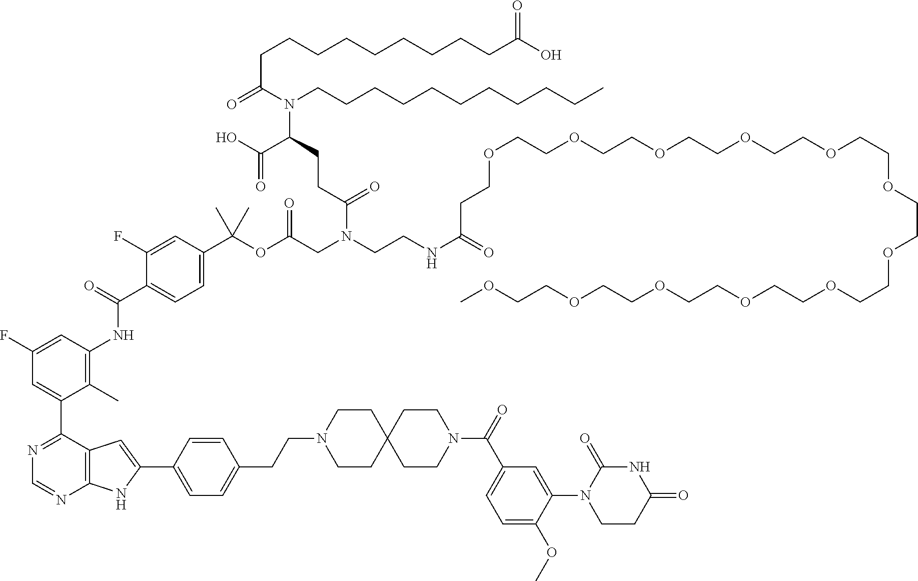 [SMILES: CCCCCCCCCCCN(C(=O)CCCCCCCCCC(=O)O)[C@@H](CCC(=O)N(CCNC(=O)CCOCCOCCOCCOCCOCCOCCOCCOCCOCCOCCOCCOC)CC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

### Step 1: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl N-(2-aminoethyl)-N-(tert-butoxycarbonyl)glycinate (Intermediate Ex. 37-1)

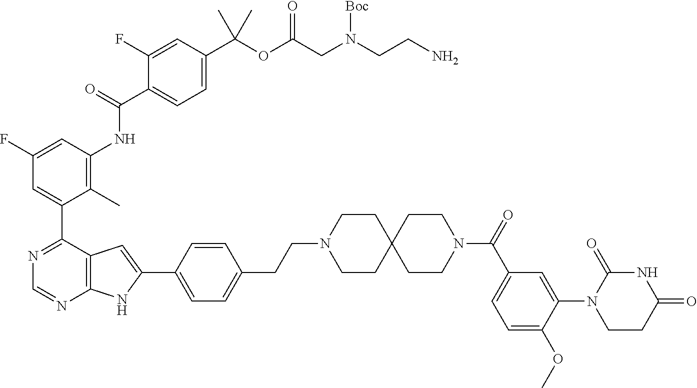 [SMILES: COc1ccc(C(=O)N2CCC3(CCN(CCc4ccc(-c5cc6c(-c7cc(F)cc(NC(=O)c8ccc(C(C)(C)OC(=O)CN(CCN)C(=O)OC(C)(C)C)cc8F)c7C)ncnc6[nH]5)cc4)CC3)CC2)cc1N1CCC(=O)NC1=O]

To a suspension of N-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide (Compound V, 1.5 g, 1.622 mmol) and N-(2-((((9H-fluoren-9-yl)methoxy)carbonyl)amino)ethyl)-N-(tert-butoxycarbonyl)glycine (0.714 g, 1.622 mmol) in DCM (3.24 mL) was added a solution of DCC (0.502 g, 2.432 mmol) and DMAP (0.263 g, 2.108 mmol) in DCM (0.5 mL) at RT. The RM was stirred at RT over the weekend. The precipitate was removed by filtration and washed with DCM. The filtrate was concentrated under reduced pressure to afford a yellow foam (1.17 g) which was dissolved in ACN/water (3/1, 10 mL) and purified by RP chromatography on a RediSep® Gold HP C18 column (275 g) eluting with ACN/water (0.1% formic acid as modifier) from 10 to 100% to afford the enriched title compound Intermediate Ex. 37-1 (150 mg) as a colorless solid. A second fraction was also isolated (57 mg) containing unreacted Compound V. The fractions were combined for step 2 (overall 207 mg of approximately 80% purity {by LCMS UV detector TAC 210-450 nm}). LCMS Method W8: Rt=0.79 min; [M−H]⁻=1123.8.

### Step 2: 2-(4-((3-(6-(4-(2-(9-(3-(2,4-Dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 42-(tert-butoxycarbonyl)-38-oxo-2,5,8,11,14,17,20,23,26,29,32,35-dodecaoxa-39,42-diazatetratetracontan-44-oate (Intermediate Ex. 37-2)

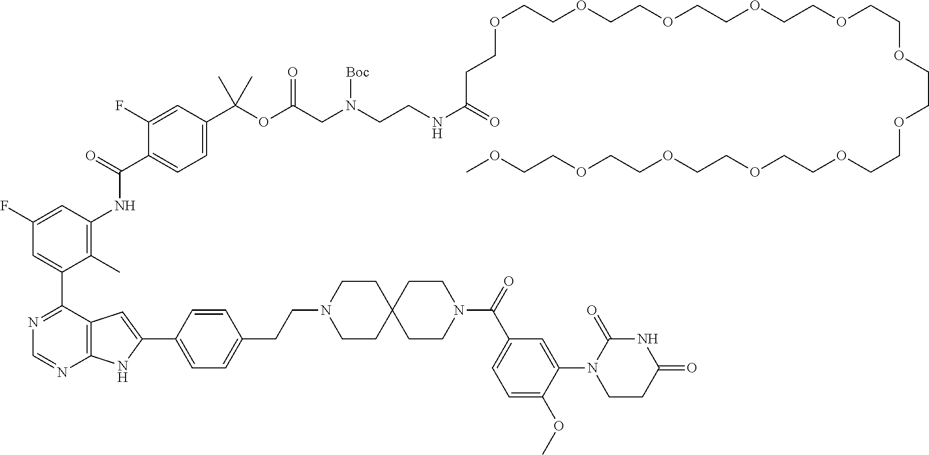 [SMILES: COCCOCCOCCOCCOCCOCCOCCOCCOCCOCCOCCOCCC(=O)NCCN(CC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)OC(C)(C)C]

To a solution of enriched 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl N-(2-aminoethyl)-N-(tert-butoxycarbonyl)glycinate (Intermediate Ex. 37-1, 207 mg) in anhydrous DMF (3 mL) at RT and under Ar atmosphere, was added 2,5-dioxopyrrolidin-1-yl 2,5,8,11,14,17,20,23,26,29,32,35-dodecaoxaoctatriacontan-38-oate (218 mg, 0.318 mmol) and DIPEA (0.064 mL, 0.368 mmol). The reaction was stirred at RT for 16.5 h and then concentrated under reduced pressure. The resulting residue was dissolved in DCM, adsorbed on silica gel and purified by chromatography over silica gel (24 g) eluting with MeOH/DCM from 0% to 20% to afford the title compound Intermediate Ex. 37-2 (170 mg) as a white foam. LCMS Method W9: Rt=4.20 min; [M+2H]²⁺=848.8.

### Step 3: Benzyl (S)-46-((benzyloxy)carbonyl)-42-(2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)-38,43,48-trioxo-47-undecyl-2,5,8,11,14,17,20,23,26,29,32,35-dodecaoxa-39,42,47-triazaoctapentacontan-58-oate (Intermediate Ex. 37-3)

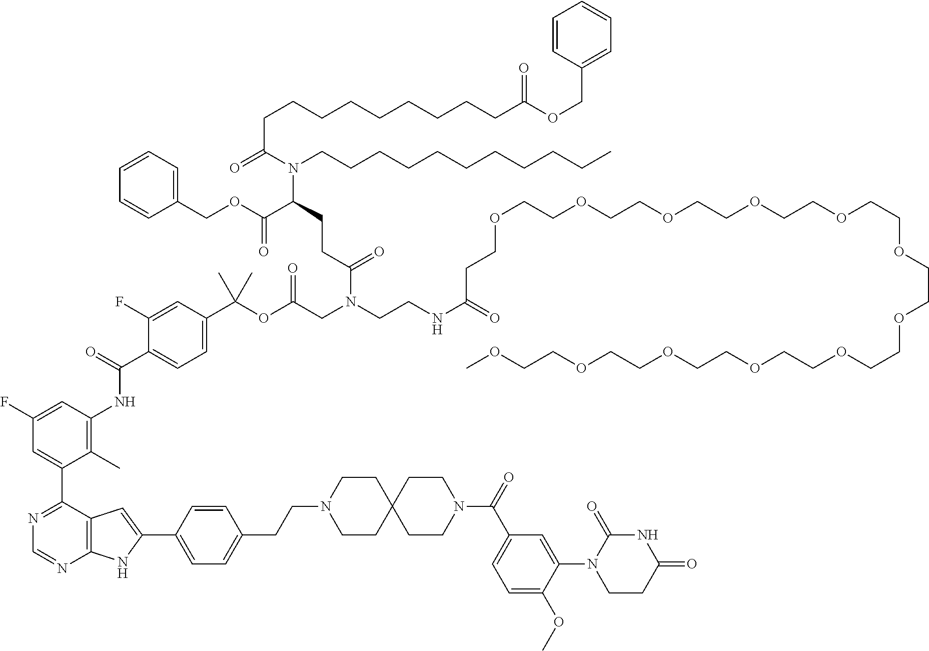 [SMILES: CCCCCCCCCCCN(C(=O)CCCCCCCCCC(=O)OCc1ccccc1)[C@@H](CCC(=O)N(CCNC(=O)CCOCCOCCOCCOCCOCCOCCOCCOCCOCCOCCOCCOC)CC(=O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)OCc1ccccc1]

To a solution of 2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl 42-(tert-butoxycarbonyl)-38-oxo-2,5,8,11,14,17,20,23,26,29,32,35-dodecaoxa-39,42-diazatetratetracontan-44-oate, (Intermediate Ex. 37-2, 170 mg, 0.100 mmol) in DCM (1 mL) was added TFA (1 mL, 12.98 mmol) at 0° C., slowly along the flask's walls. The RM was stirred at 0° C. for 1.3 h and then concentrated under reduced pressure. The residue was dissolved in anhydrous DMF (2 mL) and DIPEA (0.279 mL, 1.60 mmol) was added portionwise to reach a basic pH. The RM was flushed with Ar and 1-benzyl 5-(perfluorophenyl) N-(11-(benzyloxy)-11-oxoundecanoyl)-N-undecyl-L-glutamate (Intermediate F4-S, 180 mg, 0.213 mmol) was added. The RM was stirred at RT under Ar atmosphere for 19.5 h. DIPEA (0.150 mL, 0.859 mmol) was added and stirring was continued for additional 6.5 h. The RM was concentrated under reduced pressure. The resulting residue was chromatographed over silica gel (12 g) eluting with MeOH/DCM from 0% to 20% to afford a colorless oil. LCMS Method W9: Rt=7.49 min; [M+2H]²⁺=1129.2. The oil was taken up in EtOAc (100 mL) and washed with a 3:2 mixture of water and brine. The aq. layer was back extracted with ACN (20 mL). The combined organic layers were dried over MgSO₄, filtered, concentrated under reduced pressure. The residue was dissolved in DCM/MeOH and concentrated under reduced pressure to obtain the title compound Intermediate Ex. 37-3 (175 mg). LCMS Method W8: Rt=1.34 min; [M+2H]²⁺=1129.5.

### Step 4: (S)-46-Carboxy-42-(2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)-38,43,48-trioxo-47-undecyl-2,5,8,11,14,17,20,23,26,29,32,35-dodecaoxa-39,42,47-triazaoctapentacontan-58-oic acid (Example A37)

A solution of benzyl (S)-46-((benzyloxy)carbonyl)-42-(2-((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)-2-oxoethyl)-38,43,48-trioxo-47-undecyl-2,5,8,11,14,17,20,23,26,29,32,35-dodecaoxa-39,42,47-triazaoctapentacontan-58-oate (Intermediate Ex. 37-3, 175 mg, 0.071 mmol) in MeOH (4 mL) and THF (1.33 mL) was degassed under reduced pressure and refilled with Ar (4×). Pd/C (0.052 g, 0.049 mmol, 10 wt. %) was added and the container was degassed and then refilled with H₂ (4×). The RM was vigorously stirred at RT under H₂ atmosphere for 30 h with intermediate additions of THF (0.83 mL), MeOH (3.5 mL) and Pd/C (4×50 mg, 10 wt. %), preceded and followed by degassing/refill cycles. The RM was filtered on Celite®, rinsed several times with MeOH/THF (3/1). The filtrate was filtered evenly through three 500 mg Agilent® 6 mL PL-Thiol MP SPE cartridges. The obtained filtrate was filtered a second time evenly through three 500 mg Agilent® 6 mL PL-Thiol MP SPE cartridges, rinsing with additional MeOH/THF (3/1). The final filtrate was concentrated under reduced pressure. The residue was taken up in MeCN and water, adsorbed on Isolute® HM-N and purified by reversed phase chromatography on a RediSep® Gold HP C18 column (50 g) eluting with ACN/water (0.1% formic acid as modifier) from 5 to 100%). Pure fractions were combined, concentrated under reduced pressure to ˜1 mL, diluted with MeCN/water and filtered through a 0.2 μm PTFE filter. Lyophilization provided the title compound Example A37 (43 mg) as a white solid. LCMS Method W9: Rt=5.97 min; [M+2H]²⁺=1039.7.

The sodium salt of Example A37 was prepared according to the method described for the preparation of the sodium salt of Example A13 herein above. LCMS Method W9: Rt=5.97 min; [M+2H]²⁺1039.4.

### Example A38: 49-Carboxy-1-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-4-(5-fluoro-3-(2-fluoro-4-(2-hydroxypropan-2-yl)benzamido)-2-methylphenyl)-7H-pyrrolo[2,3-d]pyrimidin-7-yl)-3,6,46,51-tetraoxo-50-undecyl-9,12,15,18,21,24,27,30,33,36,39,42-dodecaoxa-2,5,45,50-tetraazahenhexacontan-61-oic acid

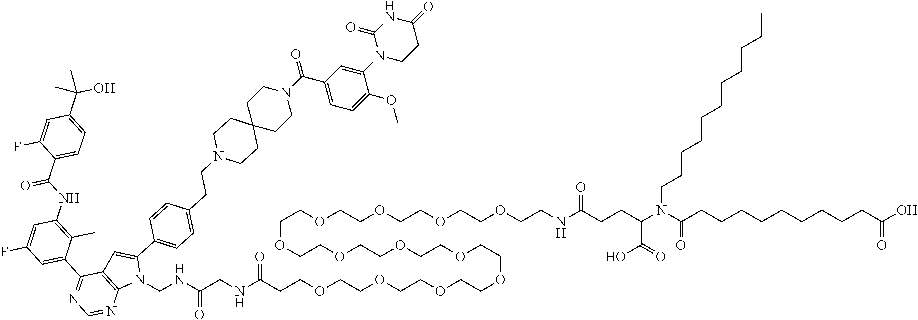 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCC(=O)NCn1c(-c2ccc(CCN3CCC4(CC3)CCN(C(=O)c3ccc(OC)c(N5CCC(=O)NC5=O)c3)CC4)cc2)cc2c(-c3cc(F)cc(NC(=O)c4ccc(C(C)(C)O)cc4F)c3C)ncnc21)C(=O)O]

Example A38 was prepared according to the method described in Example A1 herein above using Intermediate R-A1 and Intermediate F4-Peg12. LCMS Method W6: Rt=1.16 min; m/z [M−2H]²⁻=1044.4. The sodium salt of Example A38 was prepared according to the method described for the preparation of the sodium salt of Example A13 herein above. LCMS Method W7: Rt=6.14 min; m/z [M−2H]²⁻=1044.5.

### Example A39: 47-Carboxy-1-((((2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl)oxy)(hydroxy)phosphoryl)oxy)-4,44,49-trioxo-48-undecyl-7,10,13,16,19,22,25,28,31,34,37,40-dodecaoxa-3,43,48-triazanonapentacontan-59-oic acid

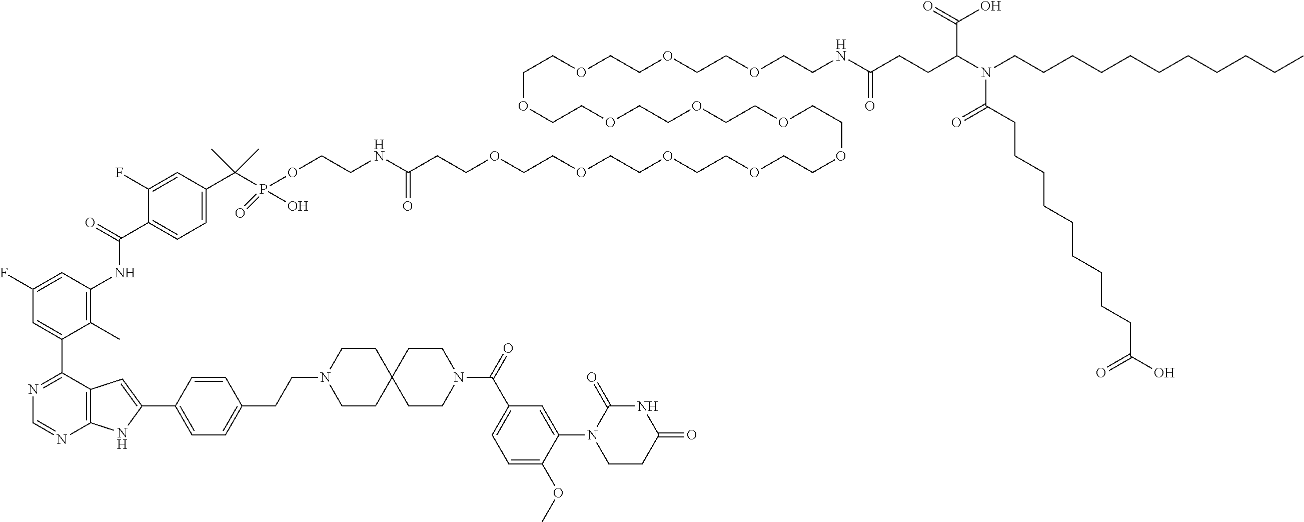 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCCOP(=O)(O)C(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O]

To a stirring solution of 2-aminoethyl (2-(4-((3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)carbamoyl)-3-fluorophenyl)propan-2-yl) hydrogen phosphate (Intermediate P-A1, 192 mg, 0.128 mmol) and DIPEA (0.112 mL, 0.641 mmol) in anhydrous DMF (5 mL) was added a solution of (S)-44-carboxy-1,41,46-trioxo-1-(perfluorophenoxy)-45-undecyl-4,7,10,13,16,19,22,25,28,31,34,37-dodecaoxa-40,45-diazahexapentacontan-56-oic acid (Intermediate F4-Peg12, 180 mg, 0.128 mmol) in anhydrous DMF (5 mL) and the RM was stirred at RT for 1 h. The RM was concentrated under reduced pressure and the crude residue was dissolved in water/MeOH (1/4, 30 mL). The solution was passed through an Agilent Stratospheres PL-Thiol MP cartridge (5 g) and the cartridge rinsed with water/MeOH (1/4, 3×50 mL) followed by a solution of water/ACN/formic acid (20/75/5, 120 mL). The combined filtrates were concentrated under reduced pressure. Purification by chromatography over C18 (RediSep® Gold, 50 g) eluting with ACN/water (0.1% formic acid as modifier) from 5-100% followed by lyophilization gave the title compound Example A39 (18 mg) as a white, fluffy powder. LCMS Method W6: Rt=1.09 min; MS m/z [M+2H]²⁺=1065.2. The sodium salt of Example A39 was prepared according to the method described for the preparation of the sodium salt of Example A13 herein above using 3 eq. of NaOH. LCMS Method W7: Rt=5.74 min; MS m/z [M−2H]²⁻=1063.2.

### Compound N: N-(3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

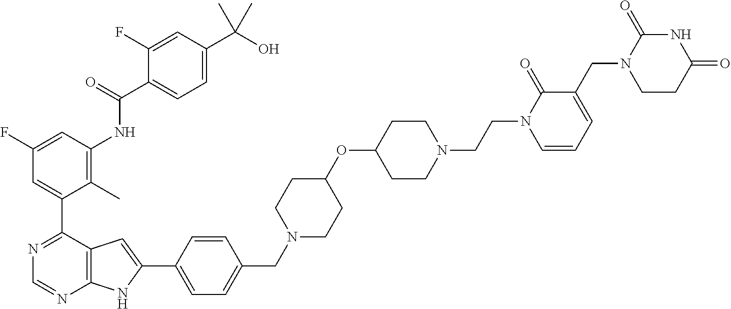 [SMILES: Cc1c(NC(=O)c2ccc(C(C)(C)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

A mixture of 1-((1-(2-(4-((1-(4-(4-chloro-7H-pyrrolo[2,3-d]pyrimidin-6-yl)benzyl)piperidin-4-yl)oxy)piperidin-1-yl)ethyl)-2-oxo-1,2-dihydropyridin-3-yl)methyl)dihydropyrimidine-2,4(1H,3H)-dione (Intermediate N5, 2.0 g, 2.97 mmol), 2-fluoro-N-(5-fluoro-2-methyl-3-(4,4,5,5-tetramethyl-1,3,2-dioxaborolan-2-yl)phenyl)-4-(2-hydroxypropan-2-yl)benzamide (Intermediate B1, 1.37 g, 3.18 mmol), cataCXium® A Pd G3 (216 mg) and aq. solution of potassium phosphate (1M, 5.9 mL) in n-butanol (20 mL) was purged with N₂ for 10 min. The RM was heated at 100° C. for 3 h and then allowed to cool to RT. The resulting mixture was filtered through a 0.45 micron membrane (Millipore) and the filtrate was concentrated under reduced pressure. The resulting residue was dissolved in DMSO (6 mL) and purified in 5 portions each on an Interchim Puriflash HQ C18 column (120 g, spherical silica, 15 μm) eluting with 0-50% ACN/water (0.1% formic acid as modifier). Fractions were combined and lyophilized for 2 days. The resulting material was diluted in minimal amount of MeOH and passed through a MP-PLO₃ carbonate resin (Agilent, 4 g, 2.14 mmol/g) eluting with MeOH and DCM. The organics were concentrated under reduced pressure and the residue was dissolved in ACN/water (1/2; 120 mL) and lyophilized providing the title compound Compound N (820 mg) as a white solid. ¹H NMR (600 MHz, DMSO-d₆; D₂O exchange) δ [ppm] 8.82 (s, 1H), 7.89 (d, J=8.0 Hz, 2H), 7.76-7.69 (m, 1H), 7.66-7.59 (m, 1H), 7.58-7.51 (m, 1H), 7.43-7.34 (m, 4H), 7.27 (d, J=6.9 Hz, 1H), 7.22 (dd, J=8.8, 2.8 Hz, 1H), 6.80 (s, 1H), 6.25-6.20 (m, 1H), 4.25 (s, 2H), 4.00-3.92 (m, 2H), 3.70-3.68 (m, 2H), 3.45 (s, 2H), 3.42-3.31 (m, 4H), 2.74-2.59 (m, 4H), 2.59-2.53 (m, 2H), 2.14 (s, 3H), 2.13-2.01 (m, 4H), 1.78-1.67 (m, 4H), 1.44 (s, 6H), 1.42-1.27 (m, 4H). LCMS Method T10: Rt=2.21 min; MS m/z [M+H]⁺=943.3.

### Compound B1: 2-((44-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)-44-methyl-39,42-dioxo-3,6,9,12,15,18,21,24,27,30,33,36,43-tridecaoxa-40-azapentatetracontyl)carbamoyl)-2-undecyltridecanedioic acid

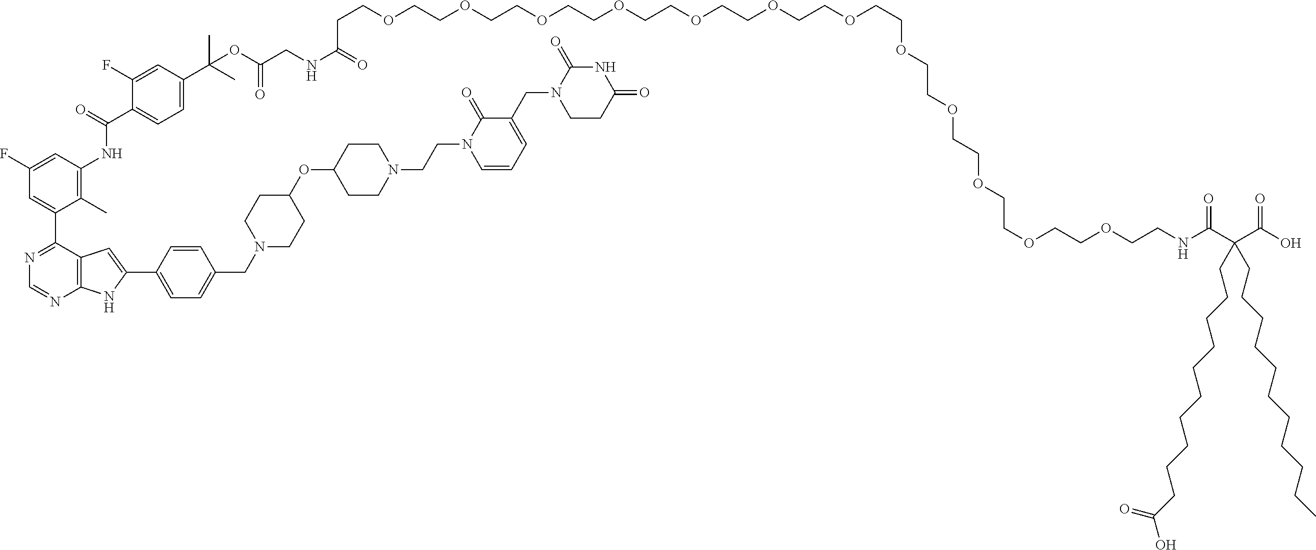 [SMILES: CCCCCCCCCCCC(CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

To a suspension of 2-(4-((3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl glycinate TFA salt (Intermediate N-A, 6.71 g, 4.20 mmol) in CH₂Cl₂ (110 mL) at 0° C. was added DIPEA (8.79 mL, 50.4 mmol). The resulting solution was stirred for 5 min and then 2-((39-((2,5-dioxopyrrolidin-1-yl)oxy)-39-oxo-3,6,9,12,15,18,21,24,27,30,33,36-dodecaoxanonatriacontyl)carbamoyl)-2-undecyltridecanedioic acid (Intermediate rac-F1-Peg12, 5.54 g, 4.62 mmol) in CH₂Cl₂ (20 mL) was added dropwise over 0.5 h. The RM was allowed to warm to RT and then stirred for 1 h at RT. The mixture was concentrated under reduced pressure and dried in high vacuum overnight. The crude material was dissolved with ACN/water (1/1, 20 mL) and purified twice using a RediSep Rf Gold® high performance C-18 Column (415 g) eluting with 0.1% formic acid as modifier in ACN/water (5 to 90%). Fractions containing the desired product were collected and most of the ACN was removed under reduced pressure. The remaining mixture was lyophilized to afford the title compound (Compound B1, 5.3 g) as a white powder. LCMS Method T8: Rt=4.06 min; MS m/z [M+2H]²⁺=1012.6. ¹H NMR (600 MHz, Methanol-d₄) δ [ppm] 8.84 (s, 1H), 7.97 (d, J=8.3 Hz, 2H), 7.90 (t, J=7.9 Hz, 1H), 7.73-7.68 (m, 1H), 7.63 (d, J=8.3 Hz, 2H), 7.59 (dd, J=6.8, 2.0 Hz, 1H), 7.51-7.47 (m, 1H), 7.40 (dd, J=8.2, 1.8 Hz, 1H), 7.35 (dd, J=12.4, 1.8 Hz, 1H), 7.19 (dd, J=8.5, 2.8 Hz, 1H), 6.89 (s, 1H), 6.38 (t, J=6.8 Hz, 1H), 4.44 (s, 2H), 4.24-4.17 (m, 4H), 3.98 (s, 2H), 3.78-3.71 (m, 3H), 3.63-3.52 (m, 49H), 3.42-3.39 (m, 2H), 3.27-3.21 (m, 2H), 3.07-2.96 (m, 4H), 2.92 (t, J=6.6 Hz, 2H), 2.67 (t, J=6.9 Hz, 2H), 2.66-2.57 (m, 2H), 2.49 (t, J=6.1 Hz, 2H), 2.23 (t, J=7.4 Hz, 2H), 2.21 (s, 3H), 2.05-1.98 (m, 2H), 1.97-1.89 (m, 2H), 1.89-1.76 (m, 12H), 1.72-1.63 (m, 2H), 1.60-1.51 (m, 2H), 1.30-1.12 (m, 32H), 0.87 (t, J=7.1 Hz, 3H) for 150 of 157 protons.

### Compound B2 and Compound B3

### (S)-2-((44-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)-44-methyl-39,42-dioxo-3,6,9,12,15,18,21,24,27,30,33,36,43-tridecaoxa-40-azapentatetracontyl)carbamoyl)-2-undecyltridecanedioic acid; and (R)-2-((44-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)-44-methyl-39,42-dioxo-3,6,9,12,15,18,21,24,27,30,33,36,43-tridecaoxa-40-azapentatetracontyl)carbamoyl)-2-undecyltridecanedioic acid

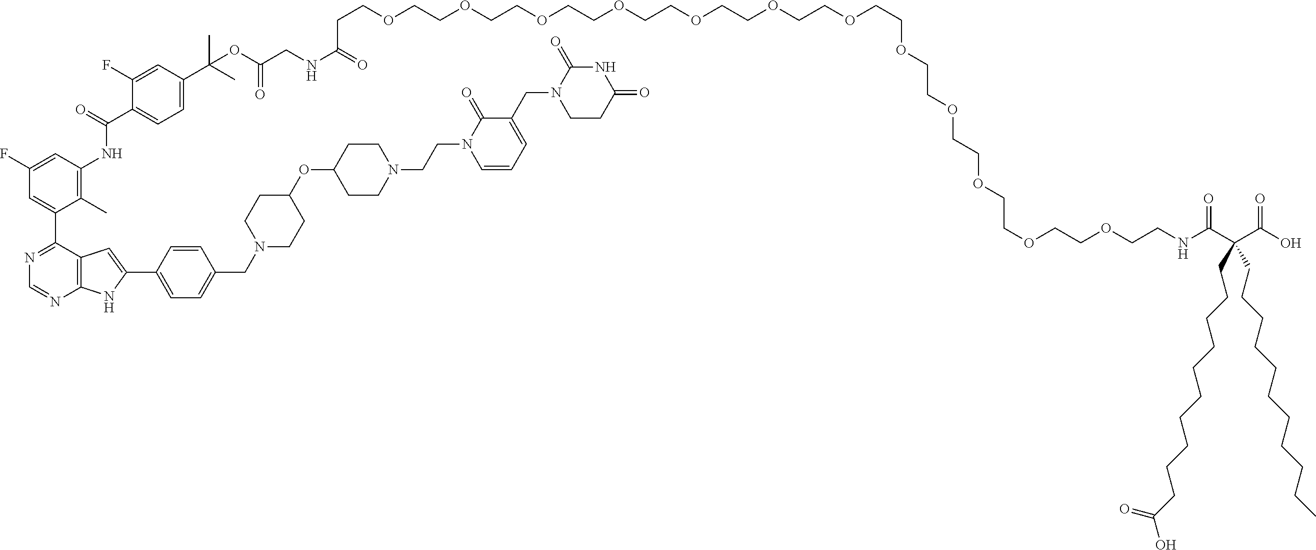 [SMILES: CCCCCCCCCCC[C@@](CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

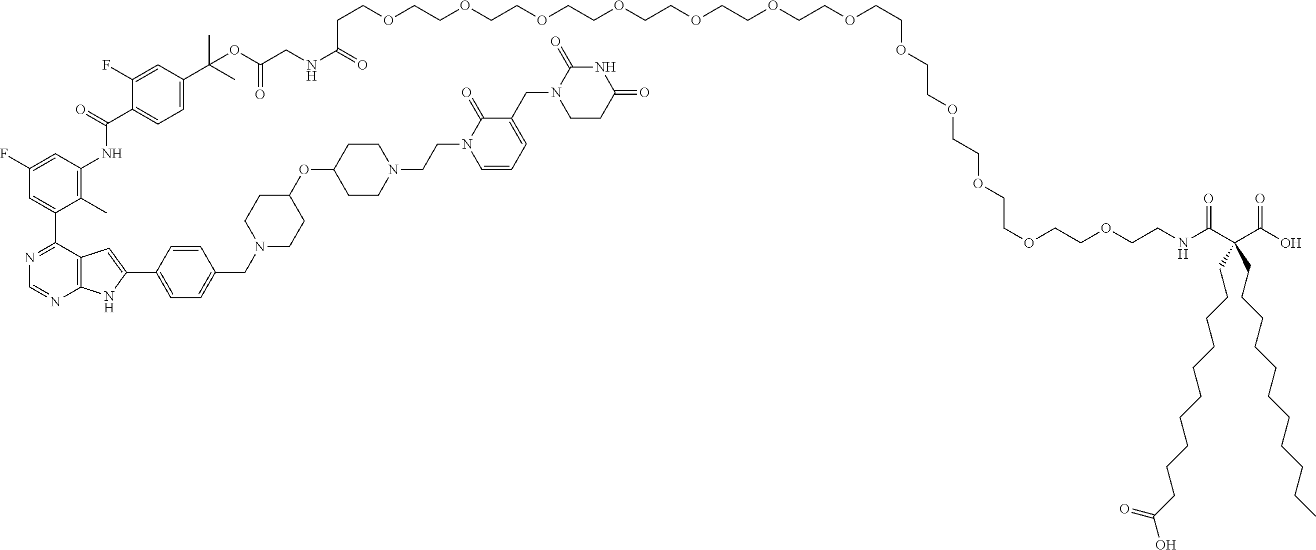 [SMILES: CCCCCCCCCCC[C@](CCCCCCCCCCC(=O)O)(C(=O)O)C(=O)NCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

### Compound B2

To a solution of 2-(4-((3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl glycinate (Intermediate N-A, 750 mg, 0.751 mmol) and DIPEA (1.31 mL) in DCM (10 mL) was added a solution of crude Intermediate ent-F1-Peak1-Peg12 (855 mg, 0.751 mmol) in DCM (10 mL). The RM was stirred for 1 h and then concentrated under reduced pressure. The resulting residue was diluted with DMSO (6 mL) and purified over an Interchim Puriflash PT C4 column (120 g, spherical silica, 15 μm, 200 Å) eluting with 10 to 60% ACN/water (0.1% formic acid as modifier) to provided enriched material after lyophilization. This material was diluted with DMSO (6 mL) and purified a second time on a C4 column (120 g) eluting with 20 to 55% ACN/water (0.1% formic acid as modifier). After lyophilization the material was re-dissolved in ACN/water (1/3) and lyophilized providing the title compound (Compound B2, 620 mg) as a white solid. LCMS Method T8: Rt=2.39 min, MS m/z [M+2H]²⁺=1013.0. ¹H NMR (400 MHz, Methanol-d₄) δ [ppm] 8.84 (s, 1H), 7.95 (d, J=8.0 Hz, 2H), 7.90 (t, J=7.9 Hz, 1H), 7.71 (d, J=9.8 Hz, 1H), 7.62 (d, J=8.1 Hz, 2H), 7.58 (dd, J=6.9, 1.9 Hz, 1H), 7.50-7.46 (m, 1H), 7.40 (dd, J=8.1, 1.7 Hz, 1H), 7.35 (d, J=12.7 Hz, 1H), 7.19 (dd, J=8.5, 2.8 Hz, 1H), 6.87 (s, 1H), 6.37 (t, J=6.8 Hz, 1H), 4.43 (s, 2H), 4.21-4.15 (m, 2H), 4.12 (s, 2H), 3.98 (s, 2H), 3.80-3.67 (m, 3H), 3.63-3.51 (m, 49H), 3.40 (t, J=5.4 Hz, 2H), 3.23-3.10 (m, 2H), 3.01-2.87 (m, 4H), 2.83 (t, J=6.6 Hz, 2H), 2.67 (t, J=6.8 Hz, 2H), 2.56-2.43 (m, 4H), 2.28-2.16 (m, 5H), 2.07-1.95 (m, 2H), 1.95-1.85 (m, 2H), 1.86-1.71 (m, 12H), 1.68-1.59 (m, 2H), 1.59-1.50 (m, 2H), 1.34-1.12 (m, 32H), 0.87 (t, J=6.8 Hz, 3H) for 150 of 157 protons. Method C7: Rt=9.89 min, ee 99.9%. Specific rotation: [□]²⁵=−4.30 (10.9 mg of Example 3 in 1 mL chloroform).

### Compound B3

To a solution of 2-(4-((3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl glycinate (Intermediate N-A, 760 mg, 0.761 mmol) and DIPEA (1.33 mL) in DCM (10 mL) was added a solution of crude Intermediate ent-F1-Peak2-Peg12 (867 mg, 0.761 mmol) in DCM (10 mL). The RM was stirred for 1 h and concentrated under reduced pressure. The resulting residue was diluted with DMSO (2 mL) and purified over an Interchim Puriflash PT C4 column (120 g, spherical silica, 15 μm, 200 Å) eluting with 5 to 50% ACN/water (0.1% formic acid as modifier) providing the title compound (Compound B3, 590 mg) as a white solid after lyophilization. LCMS Method T8: Rt=2.39 min; MS m/z [M+2H]²⁺=1013.0. ¹H NMR (400 MHz, Methanol-d₄) δ [ppm]8.84 (s, 1H), 7.96 (d, J=8.0 Hz, 2H), 7.90 (t, J=7.9 Hz, 1H), 7.71 (dd, J=9.9, 2.7 Hz, 1H), 7.62 (d, J=8.1 Hz, 2H), 7.58 (dd, J=7.5, 1.5 Hz, 1H), 7.48 (d, J=6.9 Hz, 1H), 7.44-7.37 (m, 1H), 7.35 (d, J=12.5 Hz, 1H), 7.19 (dd, J=8.5, 2.8 Hz, 1H), 6.88 (s, 1H), 6.37 (t, J=6.8 Hz, 1H), 4.43 (s, 2H), 4.22-4.14 (m, 2H), 4.12 (s, 2H), 3.98 (s, 2H), 3.77-3.68 (m, 3H), 3.64-3.50 (m, 49H), 3.40 (t, J=5.4 Hz, 2H), 3.25-3.11 (m, 2H), 3.01-2.87 (m, 4H), 2.83 (t, J=6.6 Hz, 2H), 2.67 (t, J=6.8 Hz, 2H), 2.57-2.43 (m, 4H), 2.28-2.18 (m, 5H), 2.07-1.95 (m, 2H), 1.95-1.85 (m, 2H), 1.85-1.71 (m, 12H), 1.71-1.59 (m, 2H), 1.59-1.51 (m, 2H), 1.32-1.10 (m, 32H), 0.87 (t, J=6.8 Hz, 3H) for 150 of 157 protons. Method C7: Rt=14.68 min, ee 99.6%. Specific rotation: [□]²⁵=+3.30 (10.5 mg of Example 4 in 1 mL chloroform).

### Compound B4: 2-((32-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)-32-methyl-27,30-dioxo-3,6,9,12,15,18,21,24,31-nonaoxa-28-azatritriacontyl)carbamoyl)-2-undecyltridecanedioic acid

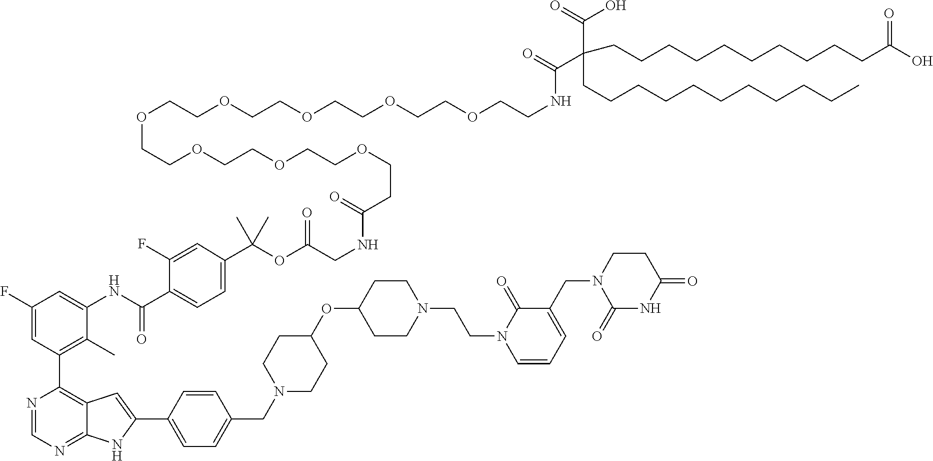 [SMILES: CCCCCCCCCCCC(CCCCCCCCCCC(=O)O)(C(=O)O)C(=O)NCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

To a light yellow suspension of 2-(4-((3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl glycinate TFA salt (Intermediate N-A, 280 mg, 0.209 mmol) in DCM (3.73 mL) was added DIPEA (270 mg, 0.365 mL, 2.09 mmol) at 0° C. The mixture was stirred for 5 min and 2-((27-((2,5-dioxopyrrolidin-1-yl)oxy)-27-oxo-3,6,9,12,15,18,21,24-octaoxaheptacosyl)carbamoyl)-2-undecyltridecanedioic acid (Intermediate rac-F1-Peg8, 231 mg, 0.240 mmol) in DCM (1.49 mL) was added dropwise. The RM was allowed to warm to RT and stirred for 1 h. The RM was diluted with water (1.5 mL) and concentrated under reduced pressure in order to remove the volatile organic solvent. The resulting residue was kept on high vacuum for ˜½ h and stored in a freezer (<0° C.) overnight. The crude residue was purified by preparative HPLC on an XBridge C18 OBD column (30×50 mm, 5 μm) eluting with 35 to 60% ACN/water modified with 0.1% formic acid [Method 5] via multiple injections to provide the title compound Compound B4 (70 mg) as a white solid. ¹H NMR (600 MHz, DMSO-d₆) δ [ppm] 12.77 (d, J=2.1 Hz, 1H), 10.15 (s, 1H), 10.04 (d, J=2.1 Hz, 1H), 8.85 (s, 1H), 8.28 (t, J=6.0 Hz, 1H), 7.94 (d, J=8.1 Hz, 2H), 7.74 (t, J=7.9 Hz, 1H), 7.64 (dd, J=10.2, 2.8 Hz, 1H), 7.57 (dd, J=6.7, 2.0 Hz, 1H), 7.41-7.34 (m, 4H), 7.27 (dd, J=6.8, 1.9 Hz, 1H), 7.24 (dd, J=8.8, 2.8 Hz, 1H), 6.83 (d, J=2.0 Hz, 1H), 6.19 (t, J=6.7 Hz, 1H), 4.26 (s, 2H), 3.98 (t, J=6.5 Hz, 2H), 3.87 (d, J=5.8 Hz, 2H), 3.59 (d, J=6.5 Hz, 2H), 3.53-3.46 (m, 4H), 3.42-3.38 (m, 6H), 3.37-3.28 (m, 26H), 3.25-3.22 (m, 2H), 2.76-2.65 (m, 4H), 2.56 (t, J=6.8 Hz, 2H), 2.52-2.48 (m, 2H), 2.37 (t, J=6.5 Hz, 2H), 2.19-2.15 (m, 5H), 2.15-2.08 (m, 4H), 1.80-1.65 (m, 14H), 1.53-1.40 (m, 4H), 1.39-1.31 (m, 2H), 1.30-1.16 (m, 28H), 1.09-0.99 (m, 4H), 0.84 (t, J=7.0 Hz, 3H) for 138 of 141 protons; 2× COOH and 1× NH not reported. LCMS Method T10: Rt=1.90 min; MS m/z [M+2H]²⁺=924.7; MS m/z [M−H]⁻=1846.2.

### Compound B5: 2-((80-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)-80-methyl-75,78-dioxo-3,6,9,12,15,18,21,24,27,30,33,36,39,42,45,48,51,54,57,60,63,66,69,72,79-pentacosaoxa-76-azahenoctacontyl)carbamoyl)-2-undecyltridecanedioic acid

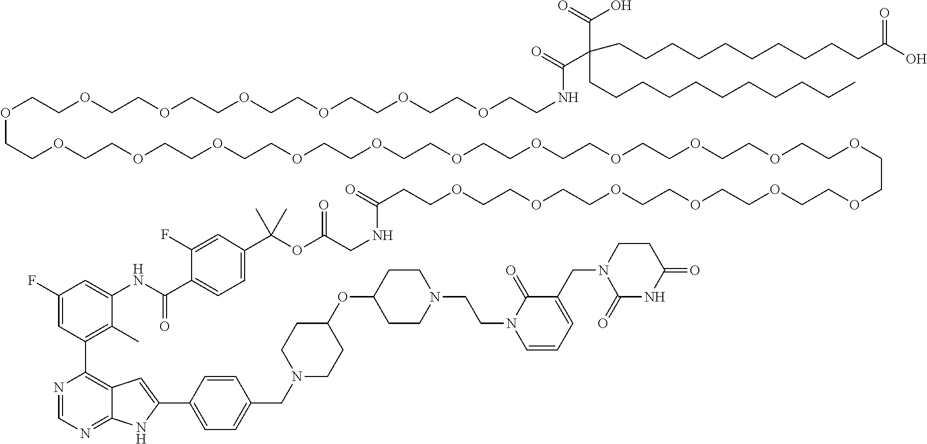 [SMILES: CCCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)NCC(=O)OC(C)(C)c1ccc(C(=O)Nc2cc(F)cc(-c3ncnc4[nH]c(-c5ccc(CN6CCC(OC7CCN(CCn8cccc(CN9CCC(=O)NC9=O)c8=O)CC7)CC6)cc5)cc34)c2C)c(F)c1]

To a light yellow suspension of 2-(4-((3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl glycinate TFA salt (Intermediate N-A, 250 mg, 0.186 mmol) in DCM (3.33 mL) was added DIPEA (241 mg, 0.326 mL, 1.86 mmol) at 0° C. The resulting mixture was stirred for 5 min and 2-((75-((2,5-dioxopyrrolidin-1-yl)oxy)-75-oxo-3,6,9,12,15,18,21,24,27,30,33,36,39,42,45,48,51,54,57,60,63,66,69,72-tetracosaoxapentaheptacontyl)carbamoyl)-2-undecyltridecanedioic acid (Intermediate rac-F1-Peg24, 231 mg, 0.240 mmol) in DCM (1.33 mL) was added dropwise. The RM was allowed to warm to RT and stirred for 1 h. The RM was diluted with water (1.5 mL) and concentrated under reduced pressure in order to remove the volatile organic solvent. The residue was kept on high vacuum for ˜½ h and stored in a freezer (<0° C.) overnight. The crude residue was purified by preparative HPLC on an XBridge C18 OBD column (30×50 mm, 5 μm) eluting with 35 to 60% ACN/water modified with 0.1% formic acid [Method 5] via multiple injections to provide the title compound Compound B5 (80 mg) as a colorless sticky solid after lyophilization. ¹H NMR (600 MHz, DMSO-d₆) δ [ppm] 12.77 (s, 1H), 10.16 (s, 1H), 10.04 (d, J=2.2 Hz, 1H), 8.85 (s, 1H), 8.27 (t, J=5.9 Hz, 1H), 8.14-8.05 (m, 1H), 8.02-7.88 (m, 2H), 7.74 (t, J=7.9 Hz, 1H), 7.64 (dd, J=10.6, 2.8 Hz, 1H), 7.57 (d, J=6.7 Hz, 1H), 7.44-7.38 (m, 2H), 7.39-7.32 (m, 2H), 7.27 (d, J=6.8 Hz, 1H), 7.24 (dd, J=8.8, 2.8 Hz, 1H), 6.92-6.76 (m, 1H), 6.20 (t, J=6.9 Hz, 1H), 4.26 (s, 2H), 4.04-3.94 (m, 2H), 3.87 (d, J=5.9 Hz, 2H), 3.60 (t, J=6.5 Hz, 2H), 3.52-3.45 (m, 94H), 3.43-3.37 (m, 6H), 3.24 (quin, J=5.9 Hz, 2H), 2.8-2.62 (m, 4H), 2.56 (t, J=6.8 Hz, 2H), 2.53-2.51 (m, 2H), 2.36 (t, J=6.5 Hz, 2H), 2.22-2.16 (m, 5H), 2.16-1.96 (m, 4H), 1.83-1.66 (m, 14H), 1.52-1.29 (m, 6H), 1.27-1.17 (m, 28H), 1.09-1.00 (m, 4H), 0.85 (t, J=6.9 Hz, 3H) for 203 of 205 protons; 2× COOH not reported. LCMS Method T10: Rt=1.85 min; MS m/z [M+2H]²⁺=1277.3; MS m/z [M−H]⁻=2550.9.

### Compound B6: 15-((44-(4-((3-(6-(4-((4-((1-(2-(3-((2,4-Dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)-44-methyl-39,42-dioxo-3,6,9,12,15,18,21,24,27,30,33,36,43-tridecaoxa-40-azapentatetracontyl)carbamoyl)nonacosane-1,15,29-tricarboxylic acid

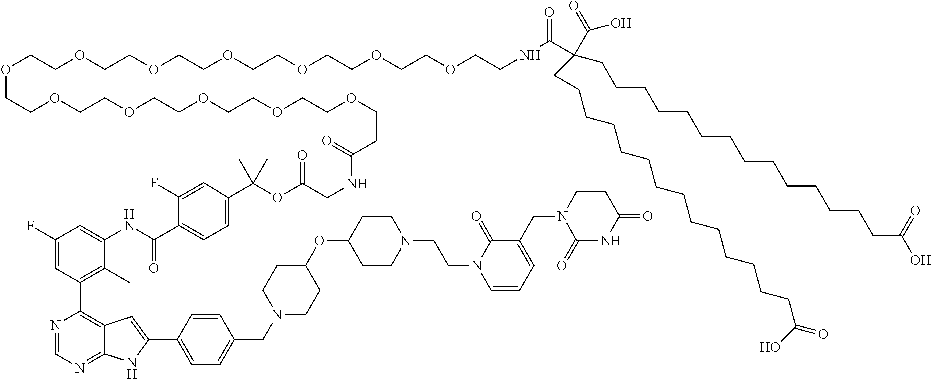 [SMILES: Cc1c(NC(=O)c2ccc(C(C)(C)OC(=O)CNC(=O)CCOCCOCCOCCOCCOCCOCCOCCOCCOCCOCCOCCOCCNC(=O)C(CCCCCCCCCCCCCCC(=O)O)(CCCCCCCCCCCCCCC(=O)O)C(=O)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

To a light yellow suspension of 2-(4-((3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)carbamoyl)-3-fluorophenyl)propan-2-yl glycinate TFA salt (Intermediate N-A, 250 mg, 0.186 mmol) in DCM (3.33 mL) was added DIPEA (241 mg, 0.326 mL, 1.86 mmol) at 0° C. The resulting mixture was stirred for 5 min and 15-((39-((2,5-dioxopyrrolidin-1-yl)oxy)-39-oxo-3,6,9,12,15,18,21,24,27,30,33,36-dodecaoxanonatriacontyl)carbamoyl)nonacosane-1,15,29-tricarboxylic acid (Intermediate F2-Peg12, 251 mg, 0.196 mmol) in DCM (1.33 mL) was added dropwise. The RM was allowed to warm to RT and stirred for 1 h. The RM was diluted with water (1.5 mL) and concentrated under reduced pressure in order to remove the volatile organic solvent. The residue was kept on high vacuum for ˜½ h and stored in a freezer (<0° C.) overnight. The crude residue was purified by preparative HPLC on an XBridge C18 OBD column (30×50 mm, 5 μm) eluting with 35 to 60% ACN/water modified with 0.1% formic acid [Method 5] via multiple injections to provide the title compound Compound B6 (68 mg) as a white solid after lyophilization. ¹H NMR (600 MHz, DMSO-d₆) δ [ppm] 12.79-12.75 (m, 1H), 10.15 (s, 1H), 10.04 (d, J=2.2 Hz, 1H), 8.85 (s, 1H), 8.28 (t, J=5.9 Hz, 1H), 7.94 (d, J=8.1 Hz, 2H), 7.74 (t, J=7.9 Hz, 1H), 7.64 (dd, J=10.2, 2.8 Hz, 1H), 7.57 (dd, J=6.8, 2.0 Hz, 1H), 7.40-7.33 (m, 4H), 7.26 (dd, J=6.7, 1.8 Hz, 1H), 7.24 (dd, J=8.8, 2.8 Hz, 1H), 6.83 (d, J=1.9 Hz, 1H), 6.19 (t, J=6.8 Hz, 1H), 4.26 (s, 2H), 3.98 (t, J=6.5 Hz, 2H), 3.87 (d, J=5.9 Hz, 2H), 3.60 (t, J=6.5 Hz, 2H), 3.51-3.46 (m, 46H), 3.43-3.37 (m, 6H), 3.25-3.21 (m, 2H), 2.77-2.63 (m, 4H), 2.56 (t, J=6.8 Hz, 2H), 2.53-2.51 (m, 2H), 2.37 (t, J=6.5 Hz, 2H), 2.19-2.15 (m, 7H), 2.14-2.05 (m, 4H), 1.81-1.64 (m, 14H), 1.51-1.30 (m, 8H), 1.25-1.16 (m, 40H), 1.09-0.97 (m, 4H) for 167 of 171 protons; 3× COOH and 1× NH not reported. LCMS Method T10: Rt=1.58 min; MS m/z [M+2H]²⁺=1084.5; MS m/z [M−H]⁻=2165.4.

### Example: BTK FAC Plasma Stability Assay

Plasma stability was conducted in Dulbecco's PBS buffer, mouse plasma, rat plasma or human plasma frozen with K3-EDTA from BiolVT). Thawed plasma was centrifuged prior to warming for 5 minutes at 37° C. Working solutions were prepared from 10 mM DMSO solution to 500□M in acetonitrile:water (1:1); 2 □L of working solution was added to 198 □L blank plasma or PBS and mixed manually. 4×45 □L of spiked matrix was aliquoted into glass incubation plates (96-well DW glass microplate) and sealed with blue silicon/PTFE seal; then incubated on a Thermo shaker (38° C. 300 RPM). Samples were prepared at 0, 2, 6 and 24 h timepoints, by protein precipitation with chilled acetonitrile containing internal standard Glyburide (0.2 □M). Precipitated samples were shaken for 10 minutes and stored at 4° C. until completion of the study. Following precipitation of the terminal sample, samples were frozen at −80° C. for 1 hour. Samples were then thawed prior to centrifugation (20 minutes, 4500 RPM at 4° C.). 50 □L of supernatant of each sample was transferred to 384 well analysis plate containing 30 □L of water. Semi quantitative analysis (analyte: internal standard peak area ratio) was performed by LC-MS/MS using a Sciex QTrap 5500.

### In Vivo Pharmacokinetic Studies

Pharmacokinetic studies were conducted in male C57/B16 mice; male Sprague-Dawley rats; male Beagle dogs and male Cynomolgus monkeys. 3 animals per group were used for each study performed. All studies were performed in accordance with local animal welfare regulations.

a. Mouse and Rat FAC PK.

Between 1 and 10 □M/kg FAC was administered either intravenously or subcutaneously as a solution in 10 mM PBS with NaOH (dose volume 5 mL/kg i.v. or 10 mL/kg s.c. in mouse and 0.5 mL/kg i.v. and 5.0 mL/kg s.c. in rat). Serial 30 □L blood samples were collected via venipuncture of the tail vein in mouse, and via the cannulated jugular vein in rats at defined timepoints to 216 hours (into EDTA coated tubes).

b. Dog and Monkey FAC PK

Between 1 and 10 □M/kg FAC was administered either intravenously or subcutaneously as a solution in 10 mM PBS with NaOH (dose volume 0.4 mL/kg in dog and monkey s.c. and 0.2 mL/kg i.v. in monkey). Serial 100 □L blood samples were collected via venipuncture of the cephalic vein at defined timepoints to 336 hours (into EDTA coated tubes).

c. Bioanalytics

Each blood sample was precipitated with acetonitrile containing internal standard; samples were mixed and centrifuged (20 minutes 4000 RPM at 4° C.). Supernatant was transferred to a microwell plate and evaporated under nitrogen at 60° C. Residue was resuspended in acetonitrile/water (7:3 v/v); plate sealed and transferred to an ultrasonic bath for 3 minutes, prior to analysis. Quantification was performed by LC-MS/MS (SciexAP16500 Q-trap, Shimadzu Nexera X2) using a Phenomenex Polar RP analytical column (2.5 μm particle size; 50×2 mm). The mobile phase consisted of solvent A (0.1% formic acid in water) and solvent B (0.1% formic acid in acetonitrile). The fatty acid conjugate and API were detected using multiple reaction monitoring.

d. Data Analysis

Data analysis and calculation of non-compartmental pharmacokinetic parameters was

performed using an in-house analysis. Following intravenous administration whole blood clearance was calculated by the dose administered divided by AUC_{0-inf}. Volume of distribution at steady state was calculated as MRT*CL, where MRT is the mean residence time, calculated by AUMC_{0-inf}/AUC_{0-inf}. The half-life (t1/2) of the terminal elimination phase was calculated by 0.693/λ. For both intravenous and subcutaneous administration, the systemic exposures were determined by calculating the area under the blood concentration time curve (AUC) from time zero to the last observed quantifiable concentration (AUCₗₐₛₜ) by using the linear trapezoidal method and normalised to dose. Extrapolated AUC (AUC_{0-inf}) is calculated by addition of AUCₗₐₛₜ and AUC_{tlast-inf}; where AUC_{tlast-inf} is calculated based on C_{last/λ}. The slope of the terminal elimination phase was estimated by linear regression of the terminal data points (minimum 3 points) from a natural log concentration versus time plot of the data. Bioavailability was calculated as the dose normalized ratio of oral AUC_{inf} to intravenous AUCinf (100*[SC AUC_{inf}/SC dose]/[IV AUC_{inf}/IV dose]). Following subcutaneous administration Cₘₐₓ is calculated as the average maximum observed peak concentration and Tₘₐₓ is the time associated with the determined Cmax.

### Pharmacology Experimental

### Animals

All animal experiments were performed according to procedures covered by permit number BS-1974, BS-1975 and BS-2904 issued by the Kantonales Veterinäramt Basel-Stadt and strictly adhered to the Eidgenössisches Tierschutzgesetz and the Eidgenössische Tierschutzverordnung. Female Sprague Dawley rat were obtained from Charles Rivers (Germany), female Balb/c mice from Charles Rivers (Italy) and female SCID/BEIGE (C.B-Igh-1b/GbmsTac-Prkdcscid-Lystbg N7) mice from Taconic. All the animals were housed in a pathogen-controlled environment with access to food and water ad libitum and they were identified with transponders.

### Tumor Models

Subcutaneous TMD8 tumors were induced by injecting tumor cells expanded in vitro in the right flank of SCID/BEIGE mice. As soon as the injection was finished, antagonization (naloxone, flumazenil and atipamezole injected s.c.) was injected and mice are put on a warming pad for recovery and carefully checked during that time.

### PK, PK/PD, Efficacy Experiments

For PK experiments, non-tumor bearing mice or rats were treated once (10 mL/kg) with a compound either i.v. or s.c. Serial blood samplings from the tail vein (5-20 μl) were collected during the course of the experiment. For PK/PD experiments in tumor-bearing mice, compound was administered once (10 mL/kg) either i.v. or s.c. Animals were randomized into groups of n=3 and tissue samples were collected at several time points. At the times indicated, animals were anaesthetized by exposure to 2-3% v/v isoflurane in medical oxygen. They were sacrificed without recovering from anesthetic after blood sampling into commercially prepared EDTA coated tubes (Milian, cat #TOM-14C) in order to extract plasma. The tissues were excised, weighted and rapidly frozen in liquid nitrogen. Later, tumors were cryogenic dry pulverized with the CryoPrep™ system (model CP-02, Covaris). For efficacy experiments in ectopic tumor-bearing mice, animals were randomized into groups of n=6 for a mean tumor size of 100 mm³ and compound was administered either i.v. q3w or s.c. q2w. When implanted in brain, animals were randomized into groups of n=4 for a mean bioluminescence of 1.0-2.0E+06 [p/s]/[ρW/cm²] and compound was administered i.v. qw. Tumor response were reported with the measures of tumor volumes or bioluminescence from the treatment start. Concentrations of compound in blood and tissues were determined simultaneously by UPLC/MS-MS. BTK and IL-10 levels in tumor were determined by Meso Scale Discovery (MSD).

### PD Screen

All animal studies were conducted in accordance with ethics and procedures covered by permit nos. BS-1975 issued by the Kantonales Veterinäramt Basel-Stadt and in strict adherence to guidelines of the Eidgenössisches Tierschutzgesetz and the Eidgenössische Tierschutzverordnung, Switzerland. All animals had access to food and water ad libitum and were identified with transponders. They were housed in a specific pathogen-free facility with a 12-h light/12-h dark cycle. For PD screen experiments in TMD8 BTK C481S mutant tumor-bearing mice, FAC was injected once. Animals were randomized into groups of 2 and tissue samples collected at day 0, 4, 7 and in some studies day 10 as well. Blood samples were collected and tumors were excised, weighed, frozen in liquid nitrogen and cryogenic dry pulverized with the CryoPrep™ system (model CP-02, Covaris). Concentrations of compounds in blood and tumors were determined by UPLC/MS-MS and BTK levels were analyzed by Meso Scale Discovery.

TABLE 8
In vitro Stability

| | Conjugate | | | | | | | | | |
|---|---|---|---|---|---|---|---|---|---|---|
| Compound | Plasma stability (h) | | | | | % Release of degrader after 24 h | | | | |
| Example | PBS | mouse | rat | dog | cyno | PBS | mouse | rat | dog | cyno |
| Compound V | >48 | 27.1 | 39.3 | >48 | — | — | — | — | — | — |
| Example 5a | | | | | | | | | | |
| Compound | | | | | | | | | | |
| Va Example | | | | | | | | | | |
| 5b | | | | | | | | | | |
| A1 | >48 | 28 | 34.8 | >48 | — | 1 | 15.1 | 17.4 | 14.6 | — |
| A2 | >48 | 12.3 | 18.1 | >48 | — | 0.8 | 3.4 | 8.9 | 7.3 | — |
| A3 | >48 | >48 | >48 | 44.4 | — | 1.9 | 5.2 | 9 | 9.7 | — |
| A12 | >48 | 27.3 | >49 | >48 | 44.9 | 0.3 | 14.2 | 2 | 1.2 | 0.5 |
| A13 | >48 | >48 | >48 | >48 | >48 | 1.4 | 3.7 | 2 | 2.7 | 2.5 |
| A14 | >48 | >48 | >48 | >48 | >48 | 0.2 | 9.5 | 0.3 | 0.1 | 0.2 |
| A15 | >48 | <2 | 30 | 23 | — | 0 | 96.9 | 39.4 | 22.5 | — |
| A16 | >48 | >48 | >48 | 31.2 | — | 2 | 1.9 | 11 | 5 | — |
| A17 | >48 | >48 | >48 | 33.3 | — | 1.2 | 2.3 | 2.5 | 3.7 | — |
| A18 | >48 | >48 | >48 | >48 | >48 | 0.6 | 0.8 | 0.2 | 0.3 | 0.4 |
| A19 | >48 | >48 | >48 | >48 | >48 | 0.3 | 0.5 | 0.5 | 0.3 | 0.2 |
| A20 | >48 | >48 | >48 | >48 | >48 | 0.2 | 0.5 | 0.3 | 0.4 | 0.2 |
| A21 | >48 | >48 | >48 | >48 | >48 | 0.2 | 1.9 | 0.5 | 0.3 | 0.4 |
| A22 | >48 | >48 | >48 | >48 | >48 | 1.3 | 2.3 | 1.4 | 1.6 | 1.2 |
| A23 | >48 | >48 | >48 | >48 | >48 | 0.2 | 5.8 | 1.1 | 0.8 | 0.6 |
| A5 | >48 | >48 | >48 | >48 | >48 | 0.1 | 2 | 2.5 | 1 | 0.5 |
| A24 | >48 | >48 | >48 | >48 | — | 2.8 | 7.4 | 7.2 | 5.9 | — |
| A25 | >48 | 17.8 | 35.7 | >48 | >48 | 2.2 | 20.9 | 1.5 | 1.1 | 1.1 |
| A26 | >48 | 30.5 | >48 | 39.2 | — | 7 | 20.4 | 7.6 | 10.9 | — |
| A27 | >48 | 28.6 | — | >48 | 26.6 | 0.3 | 13.7 | 2 | 0.5 | 0.4 |
| A36 | >48 | 38.6 | >48 | >48 | — | 0 | 0.7 | 0 | 0 | — |
| A38 | >48 | >48 | >48 | >48 | — | 0.2 | 0.1 | 0.1 | 0.1 | — |
| A37 | >48 | >48 | >48 | 44.7 | — | 1.7 | 2.1 | 1.5 | 2.2 | — |
| A39 | >48 | >48 | >48 | 39.0 | — | 0 | 0 | 0 | 0 | — |
| A6 | >48 | 10.7 | 36.6 | 12.7 | — | 0.4 | 37.4 | 21.1 | 4.6 | — |
| A7 | >48 | 10.1 | — | — | — | 1.5 | 53.6 | — | — | — |
| A8 | >48 | >48 | >48 | >48 | — | 2.8 | 20.1 | 18.1 | 13.1 | — |
| A9 | >48 | 4.6 | 6.8 | >48 | 30.8 | 1.2 | 15.6 | 22.4 | 5.7 | 3.7 |
| A32 | >48 | 27 | 20.8 | 37.3 | — | 1 | 5.8 | 6.1 | 4.7 | — |
| A33 | >48 | 5.1 | 2.3 | 32 | 45.7 | 4.8 | 27.7 | 16.7 | 10.4 | 8.1 |
| A34 | >48 | 6.8 | 5.3 | >48 | 30.6 | 5.2 | 26.2 | 42.2 | 5.3 | 13.9 |
| A31 | >72 | <1 | 1.2 | 33.6 | — | — | — | — | — | — |
| A30 | >72 | <1 | — | — | — | — | — | — | — | — |
| A10 | — | — | — | — | — | — | — | — | — | — |
| A11 | — | — | — | — | — | — | — | — | — | — |
| A28 | — | — | — | — | — | — | — | — | — | — |
| A29 | — | — | — | — | — | — | — | — | — | — |

TABLE 9
Cross species PK-AUC

| | FAC | | | | Payload | | | |
|---|---|---|---|---|---|---|---|---|
| PK parameter | AUC-uM.h | | | | AUC-uM.h | | | |
| Species | mouse | rat | dog | cyno | mouse | Rat | dog | cyno |
| FAC i.v. dose | 10 | 10 | 1 | 1 | | | | |
| (umol/kg) | | | | | | | | |
| API i.v. dose | | | | | 10 | 10 | 1 | 1 |
| (umol/kg) | | | | | | | | |
| Compound V | NA | | | | 8.2 | 0.57 | | |
| (1 umol/kg) | | | | | | | | |
| Compound Va | NA | | | | 15.2 | | | |
| Example 5b | | | | | | | | |
| (20 mg/kg) | | | | | | | | |
| Example A1 | 1112 | 852/647 | 6.8 | | 10 | 4.0/14 | | |
| Example A2 | | 161 | | 590 | | 2.18 | 2.1 | |
| Example A3 | | | — | — | | | — | — |
| Example A12 | 1000 | 672 | — | — | | 0.2 | | |
| Example A13 | 1214 | 593 | — | — | 4.2 | 2.6 | | |
| Example A14 | 696 | | — | — | 0.54 | | | |
| Example A15 | 397 | | — | — | 5.1 | | | |
| Example A16 | 47 | 800 | — | — | 0.8 | 9.8 | | |
| (25 umol/kg) | | | | | | | | |
| Example A17 | | | — | — | | | | |
| Example A18 | 128 | | — | — | | | 91 | |
| Example A19 | 2953 | | — | — | 2.3 | | | |
| Example A20 | | | — | — | | | | |
| Example A21 | | | — | — | | | | |
| Example A22 | | | — | — | | | | |
| Example A23 | 856 | | — | — | | | 123 | |
| Example A5 | 3682 | 2228 | — | — | 11.6 | 0.2 | | |
| Example A24 | | | — | — | | | | |
| Example A25 | 974 | | — | — | 1.5 | | | |
| Example A26 | | 826 | — | — | | 0.43 | | |
| Example A27 | | | — | — | | | | |
| Example A36 | | 1397 | — | — | | 0.2 | | |
| (25 umol/kg) | | | | | | | | |
| Example A38 | | | — | — | | | | |
| Example A37 | | | — | — | | | | |
| Example A39 | | | — | — | | | | |
| Example A6 | | | — | — | | | | |
| Example A7 | 293 | | — | — | 5.5 | | | |
| Example A8 | | | — | — | | | | |
| Example A9 | | | — | — | | | | |
| Example A32 | | | — | — | | | | |
| Example A33 | | | — | — | | | | |
| Example A34 | | | — | — | | | | |
| Example A31 | | | — | — | | | | |
| Example A30 | | | — | — | | | | |
| Example A10 | 2762 | 975 | | | 163 | 3.1 | | |
| Example A11 | | | | | | | | |
| Example A28 | | 704 | | | | 3.6 | | |
| Example A29 | | | | | | | | |

TABLE 10
Cross species PK

| PK | Fatty Acid Conjugate | | | | | | | |
|---|---|---|---|---|---|---|---|---|
| Parameter | AUC-uM.h d.n. | | | | C₀-uM d.n. | | | |
| Species | Mouse | Rat | Dog | Cyno | Mouse | Rat | Dog | Cyno |
| Conj. i.v. | 40 | 40 | 10 | 1 | 40 | 40 | 10 | 1 |
| dose | | | | | | | | |
| (umol/kg) | | | | | | | | |
| Compound N | N/A | N/A | N/A | N/A | N/A | N/A | N/A | N/A |
| (unconj. BTK | | | | | | | | |
| Degrader) | | | | | | | | |
| Compound | 71 | 35 | 30 | 33 | 3.3 | 1.8 | 3.3 | 9.9 |
| B1 (conj) | | | | | | | | |
| Compound | 80 | 21 | — | — | 4.8 | 1.8 | — | — |
| B2 (conj) | | | | | | | | |
| Compound | 195 | 77 | — | — | 4.9 | 3.8 | — | — |
| B3 (conj) | | | | | | | | |
| Compound | — | 14 | — | — | — | 1.7 | — | — |
| B4 (conj) | | | | | | | | |
| Compound | — | 39 | — | — | — | 2.5 | — | — |
| B5 (conj) | | | | | | | | |
| Compound | — | 15 | — | — | — | 1.2 | — | — |
| B6 (conj) | | | | | | | | |

TABLE 11
Cross species PK-C₀

| | FAC | | | | Payload | | | |
|---|---|---|---|---|---|---|---|---|
| PK parameter | C₀-uM | | | | C₀-uM | | | |
| Species | mouse | rat | dog | cyno | mouse | Rat | dog | cyno |
| FAC/i.v. dose | 10 | 10 | 1 | 1 | 10 | 10 | 1 | 1 |
| (umol/kg) | | | | | | | | |
| API i.v. dose | | | | | | | | |
| (umol/kg) | | | | | | | | |
| Compound V | NA | | | | 8.1 | 1.1 | | |
| (1 umol/kg) | | | | | | | | |
| Compound Va | NA | | | | 16.7 | | | |
| Example 5b | | | | | | | | |
| (20 mg/kg) | | | | | | | | |
| Example A1 | 96 | 146/106 | 27.2 | | 8.1 | | | |
| Example A2 | | 113 | | 56.6 | | 1.1 | 0.2 | |
| Example A3 | — | — | — | — | — | — | — | — |
| Example A12 | 102 | 125 | — | — | | 0.03 | | |
| Example A13 | 143 | 145 | — | — | 0.05 | 0.03 | | |
| Example A14 | 115 | | — | — | 0.3 | | | |
| Example A15 | 99 | | — | — | 0.02 | | | |
| Example A16 | 4 | 94 | — | — | 0.03 | 1.3 | | |
| (25 umol/kg) | | | | | | | | |
| Example A17 | | | — | — | | | | |
| Example A18 | | | — | — | | | 0.3 | |
| Example A19 | 169 | | — | — | 0.2 | | | |
| Example A20 | | | — | — | 0.06 | | | |
| Example A21 | | | — | — | | | | |
| Example A22 | | | — | — | | | | |
| Example A23 | | | — | — | | | 0.59 | |
| Example A5 | 109 | 152 | — | — | 0.016 | | | |
| Example A24 | | | — | — | 0.16 | 0.04 | | |
| Example A25 | 108 | | — | — | | | | |
| Example A26 | | 229 | — | — | 0.03 | | | |
| Example A27 | | | — | — | | 0.08 | | |
| Example A36 | | 123 | — | — | | 0.06 | | |
| (25 umol/kg) | | | | | | | | |
| Example A38 | | | — | — | | | | |
| Example A37 | | | — | — | | | | |
| Example A39 | | | — | — | | | | |
| Example A6 | | | — | — | | | | |
| Example A7 | 93 | | — | — | 0.7 | | | |
| Example A8 | | | — | — | | | | |
| Example A9 | | | — | — | | | | |
| Example A32 | | | — | — | | | | |
| Example A33 | | | — | — | | | | |
| Example A34 | | | — | — | | | | |
| Example A31 | | | — | — | | | | |
| Example A30 | | | — | — | | | | |
| Example A10 | 119 | 116 | | | 7 | 0.6 | | |
| Example A11 | | | | | | | | |
| Example A28 | | 57 | | | | 0.21 | | |
| Example A29 | | | | | | | | |

TABLE 12
Cross species PK (Payload)

| | Payload (BTK Degrader) | | | | | | | |
|---|---|---|---|---|---|---|---|---|
| PK Parameter | AUC-uM.h d.n. | | | | C₀/Cₘₐₓ uM d.n. | | | |
| Species | Mouse | Rat | Dog | Cyno | Mouse | Rat | Dog | Cyno |
| Conj. i.v. dose | 40 | 40 | 10 | 1 | 40 | 40 | 10 | 1 |
| (umol/kg) | | | | | | | | |
| API i.v. dose | 37.7 | 18.8 | 0.9 | 0.9 | 37.7 | 18.8 | 0.9 | 0.9 |
| (umol/kg) | | | | | | | | |
| Compound N | 1.6 | 0.89 | 1.2 | 3.4 | 0.36 | 0.237 | 0.95 | 1.76 |
| (unconj. BTK | | | | | | | | |
| Degrader | | | | | | | | |
| Compound B1 | 1.2 | 0.43 | 1.3 | 0.7 | 0.04 | 0.030 | 0.07 | 0.04 |
| (conj) | | | | | | | | |
| Compound B2 | 0.6 | 0.23 | — | — | 0.04 | 0.010 | — | — |
| (conj) | | | | | | | | |
| Compound B3 | 0.4 | 0.20 | — | — | 0.001 | 0.010 | — | — |
| (conj) | | | | | | | | |
| Compound B4 | — | 0.04 | — | — | — | 0.001 | — | — |
| (conj) | | | | | | | | |
| Compound B5 | — | 0.20 | — | — | — | 0.005 | — | — |
| (conj) | | | | | | | | |
| Compound B6 | — | 0.08 | — | — | — | 0.002 | — | — |
| (conj) | | | | | | | | |

TABLE 13
Subcutaneous dosing

| | FAC | | | | Payload | | | |
|---|---|---|---|---|---|---|---|---|
| PK parameter | AUC-uM.h | | | | AUC-uM.h | | | |
| Species | mouse | rat | dog | cyno | mouse | Rat | dog | cyno |
| FAC s.c. dose | 10 | 10 | 1 | 1 | | | | |
| (umol/kg) | | | | | | | | |
| API s.c. dose | | | | | 10 | 10 | 1 | 1 |
| (umol/kg) | | | | | | | | |
| Compound V | NA | | | | 8.2 | 0.57 | | |
| Example 5a | | | | | | | | |
| (1 umol/kg) | | | | | | | | |
| Compound Va | NA | | | | 15.2 | | | |
| Example 5b | | | | | | | | |
| (20 mg/kg) | | | | | | | | |
| Example A1 | | | | | 10 | 4.0/14 | | |
| Example A5 | | | | | 11.6 | 0.2 | | |
| Example A28 | | | | | | | | |

TABLE 14
Subcutaneous dosing

| | FAC | | | | Payload | | | |
|---|---|---|---|---|---|---|---|---|
| PK parameter | C₀-uM | | | | C₀-uM | | | |
| Species | mouse | rat | dog | cyno | mouse | Rat | dog | cyno |
| FAC s.c. dose | 10 | 10 | 1 | 1 | 10 | 10 | 1 | 1 |
| (umol/kg) | | | | | | | | |
| API i.v. dose | | | | | | | | |
| (umol/kg) | | | | | | | | |
| Compound V | NA | | | | NA | NA | NA | NA |
| Example 5a | | | | | | | | |
| (1 umol/kg) | | | | | | | | |
| Compound Va | NA | | | | NA | NA | NA | NA |
| Example 5b | | | | | | | | |
| (20 mg/kg) | | | | | | | | |
| Example A1 | | | 0.28 | | | | 0.496 | |
| Example A5 | | | | | | | | |
| Example A28 | | | | | | | | |

*doses specified are doses of FAC.

TABLE 15
Cross species PK s.c. Lipophilic Conjugate

| PK | Lipophilic Acid Conjugate | | | | | | | |
|---|---|---|---|---|---|---|---|---|
| Parameter | AUC-uM.h | | | | Cₘₐₓ-uM | | | |
| Species | Mouse | Rat | Dog | Cyno | Mouse | Rat | Dog | Cyno |
| Conj. s.c | 40 | 40 | 10 | 1 | 40 | 40 | 10 | 1 |
| dose | | | | | | | | |
| (umol/kg) | | | | | | | | |
| Compound N | N/A | N/A | N/A | N/A | N/A | N/A | N/A | N/A |
| (unconj. BTK | | | | | | | | |
| Degrader) | | | | | | | | |
| Compound | 3860 | 667 | 304 | 26.1 | 71 | 11 | 37.5 | 3.8 |
| B1 (conj) | | | | | | | | |
| Compound | 1859 | — | — | — | 60 | — | — | — |
| B2 (conj) | | | | | | | | |
| Compound | 3847 | — | — | — | 68 | — | — | — |
| B3 (conj) | | | | | | | | |

TABLE 16
Cross species PK s.c. Payload

| PK | Payload (BTK Degrader) | | | | | | | |
|---|---|---|---|---|---|---|---|---|
| Parameter | AUC-uM.h | | | | Cₘₐₓ uM | | | |
| Species | Mouse | Rat | Dog | Cyno | Mouse | Rat | Dog | Cyno |
| Conj. s.c. | 40 | 40 | 10 | 1 | 40 | 40 | 10 | |
| dose | | | | | | | | |
| (umol/kg) | | | | | | | | |
| API s.c. dose | 10 | — | — | — | 10 | 18.8 | 0.9 | 0.9 |
| (umol/kg) | | | | | | | | |
| Compound N | 9.3 | — | — | — | 1.6 | — | — | — |
| (unconj. BTK | | | | | | | | |
| Degrader (i.v.) | | | | | | | | |
| Compound | 51 | 12.2 | 12.8 | 0.01 | 0.6 | 0.2 | 0.7 | 0.7 |
| B1 (conj) | | | | | | | | |
| Compound | 23 | — | — | — | 0.5 | — | — | — |
| B2 (conj) | | | | | | | | |
| Compound | 25 | — | — | — | 0.3 | — | — | — |
| B3 (conj) | | | | | | | | |

TABLE 17
Extension of PD and efficacy

| | Duration of BTK | | Dosing frequency | |
|---|---|---|---|---|
| | degradation in | | to achieve sustained | |
| | mouse ≥90% (days) | | 90% regression | |
| | (25 umol/kg) | | (25 umol/kg iv) | |
| Compound | i.v. | s.c. | i.v. | s.c. |
| V | <4 | Not tolerated | 2qw (85%) | Not tolerated |
| Example 5a | | | | |
| (20 mgL/kg) | | | | |
| Example A1 | 7 | | q3w | q3w |
| Example A13 | 7 | — | — | — |
| (50 umol/kg) | | | | |
| Example A16 | 4 | — | — | — |
| Example A17 | 4 | — | — | — |
| Example A18 | <4 | — | — | — |
| (50 umol/kg) | | | | |
| Example A19 | 7* | — | — | — |
| (50 umol/kg) | | | | |
| Example A20 | 7* | — | — | |
| (50 umol/kg) | | | | |
| Example A21 | <4 | — | — | |
| (50 mol/kg) | | | | |
| Example A22 | <4 | — | — | |
| (50 umol/kg) | | | | |
| Example A23 | 4 | — | — | |
| (50 umol/kg) | | | | |
| Example A5 | 10 | 10 | — | |
| Example A24 | 10 | 10 | — | |
| Example A25 | — | — | — | |
| Example A26 | 7 | — | — | |
| Example A36 | 10 | — | — | |
| Example A8 | 4 | — | — | |
| Example A10 | — | — | — | — |
| Example A11 | — | — | — | — |
| Example A28 | 10 | 10 | — | — |
| Example A29 | — | — | — | — |

TABLE 18
Extension of PD and efficacy

| | Duration of BTK | | | |
|---|---|---|---|---|
| | degradation in | | Dosing to achieve | |
| | mouse ≥90% (days) | | sustained ~90% | |
| | I.v. (40 | | regression | |
| Compound | umol/kg) | s.c. | I.v. | s.c. |
| N (20 mg/kg) | 4 | 1(10 mg/kg) | 20 mg/kg 2qw | NA |
| B1 | 10 | — | 40 umol/kg q3w | — |
| B2 | 10 | — | 60 umol/kg q3w | — |
| B3 | 14 | — | 20 umol/kg q3w | — |

These data shows that the fatty acid conjugates of the disclosure deliver the payload (the unconjugated BTK degrader compound) over an extended period compared to direct administration of the unconjugated BTK degraders, thus enabling less frequent dosing, which is more convenient and comfortable to the patient.

Those skilled in the art will recognize, or be able to ascertain, using no more than routine experimentation, numerous equivalents to the specific embodiments described specifically herein. Such equivalents are intended to be encompassed in the scope of the following claims.

1. A conjugate of Formula (I): 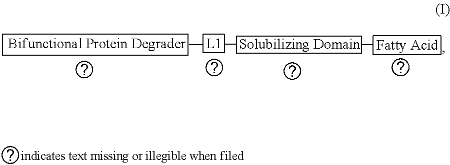
or a pharmaceutically acceptable salt, hydrate, solvate, prodrug, stereoisomer, or tautomer thereof, wherein
(i) a bifunctional protein degrader comprises a bifunctional compound capable of binding to each of a target protein and a ligase independently;
(ii) L1 comprises a cleavable linker;
(iii) optionally, a solubilizing domain comprises a heteroalkylene and is soluble in aqueous solution; and
(iv) a fatty acid comprises a fatty acid capable of binding to a protein.

2. A conjugate of Formula (I′): 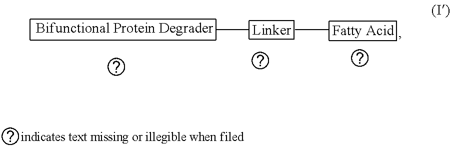
or a pharmaceutically acceptable salt, hydrate, solvate, stereoisomer, or tautomer thereof, wherein
a bifunctional protein degrader is a Bruton's Tyrosine Kinase (BTK) Degrader capable of degrading BTK; and
a Linker is absent or L⁴, wherein L⁴ is a group that is cleavable to allow release of the Bifunctional Protein Degrader, and that covalently links the Bifunctional Protein Degrader to a Fatty Acid.

3. The conjugate, or a pharmaceutically acceptable salt, hydrate, solvate, prodrug, stereoisomer, or tautomer thereof, according to claim 2, wherein L⁴ comprises L1 and, optionally, a solubilizing domain.

4. The conjugate, or a pharmaceutically acceptable salt, hydrate, solvate, prodrug, stereoisomer, or tautomer thereof, according to any one of claims 1-3, wherein the bifunctional protein degrader has the structure of Formula (I-a): 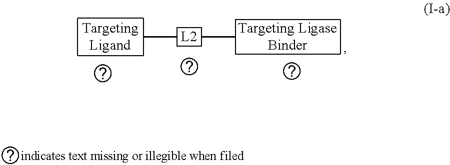
or a pharmaceutically acceptable salt, hydrate, solvate, prodrug, stereoisomer, or tautomer thereof, wherein:
(i) a targeting ligand comprises an entity capable of binding to the target protein;
(ii) L2 is a linker; and
(iii) a targeting ligase binder comprises an entity capable of binding the ligase.

5. The conjugate, or a pharmaceutically acceptable salt, hydrate, solvate, prodrug, stereoisomer, or tautomer thereof, according to claim 4, wherein the targeting ligase binder has the structure of Formula (TLB-I): 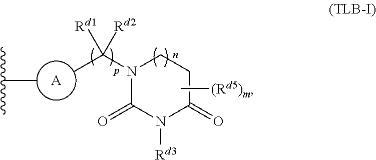
wherein: 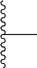
denotes the point of attachment to L2 in Formula (I-a);
Ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl; or
two R^{d5} together with the carbon atoms to which they are attached form a cycloalkyl; or
two R^{d5} attached to the same carbon atom form a C₃₋₄ spirocycloalkyl;
R^{p} is H or C₁₋₆ alkyl;
m is 1 or 2;
n is 1 or 2; and
p is 0, 1, or 2.

6. The conjugate, or a pharmaceutically acceptable salt, hydrate, solvate, prodrug, stereoisomer, or tautomer thereof, according to claim 5, wherein ring A is a 5-membered nitrogen-containing heteroaryl or a 6-membered nitrogen-containing heteroaryl.

7. The conjugate, or a pharmaceutically acceptable salt, hydrate, solvate, prodrug, stereoisomer, or tautomer thereof, according to claim 5 or 6, wherein ring A is selected from the group consisting of phenyl, pyridyl, pyridonyl, pyrazinyl, thiophenyl, pyrazolyl, imidazolyl, and pyrrolyl.

8. The conjugate, or a pharmaceutically acceptable salt, hydrate, solvate, prodrug, stereoisomer, or tautomer thereof, according to any one of claims 5-7, wherein the targeting ligase binder of Formula (TLB-I) has a structure selected from the group consisting of Formulas (TLB-I-i), (TLB-I-ii), and (TLB-I-iii): 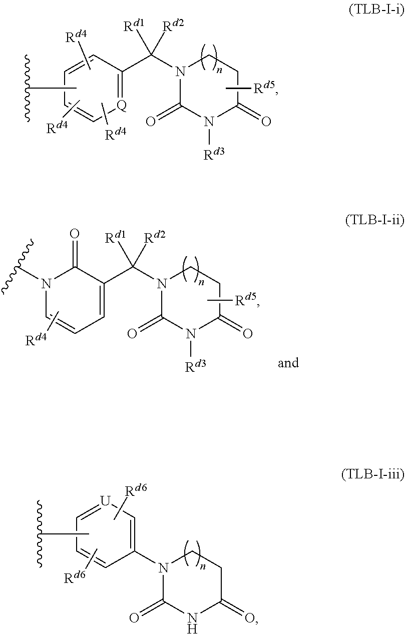
wherein: 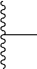
denotes the point of attachment to L2 in Formula (I-a);
Q is N or CR^{d4};
U is —CR^{d6} or N;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
R^{d5} is selected from the group consisting of H, C₁₋₆ alkyl, halogen, C₁₋₆ haloalkyl, and C₁₋₆ heteroalkyl;
each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl;
R^{p} is H or C₁₋₆ alkyl; and
n is 1 or 2.

9. The conjugate, or a pharmaceutically acceptable salt, hydrate, solvate, prodrug, stereoisomer, or tautomer thereof, according to any one of claims 5-8, wherein n is 1.

10. The conjugate, or a pharmaceutically acceptable salt, hydrate, solvate, prodrug, stereoisomer, or tautomer thereof, according to any one of claims 5-9, wherein R^{d3} is H or —CH₂OP(O)(OR^{p})₂.

11. The conjugate, or a pharmaceutically acceptable salt, hydrate, solvate, prodrug, stereoisomer, or tautomer thereof, according to any one of claims 5-10, wherein R^{d1} is H.

12. The conjugate, or a pharmaceutically acceptable salt, hydrate, solvate, prodrug, stereoisomer, or tautomer thereof, according to any one of claims 5-11, wherein R^{d2} is H.

13. The conjugate, or a pharmaceutically acceptable salt, hydrate, solvate, prodrug, stereoisomer, or tautomer thereof, according to any one of claims 5-12, wherein R^{d1} and R^{d2} are both independently H.

14. The conjugate, or a pharmaceutically acceptable salt, hydrate, solvate, prodrug, stereoisomer, or tautomer thereof, according to any one of claims 4-13, wherein L2 has a structure of Formula (L-I): 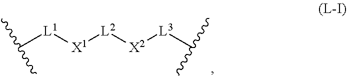
wherein:
L¹ is selected from the group consisting of a bond, O, NR′, C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the targeting ligand in Formula (I-a);
X¹ and X² are each independently selected from the group consisting of a bond, carbocyclyl, heterocyclyl, and heteroaryl, wherein the carbocyclyl, heterocyclyl, and heteroaryl are each substituted with 0-4 occurrences of R^{a}, wherein each R^{a} is independently selected from the group consisting of C₁₋₆ alkyl, C₁₋₆ alkoxyl, C₁₋₆ hydroxyalkyl, and halogen;
L² is selected from the group consisting of a bond, O, NR′, C(O), C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl substituted with 0-4 occurrences of R^{b}, wherein each R^{b} is independently selected from the group consisting of C₁₋₆ alkyl, C₁₋₆ alkoxyl, C₁₋₆ hydroxyalkyl, and halogen;
L³ is selected from a bond, C₁₋₆ alkylene, C₂₋₆ alkenylene, C₂₋₆ alkynylene, C₁₋₆ heteroalkylene, C(O), S(O)₂, O, NR′, *C(O)—C₁₋₉ alkylene, *C(O)—C₁₋₆ alkylene-O, and *C(O)—C₁₋₉ heteroalkylene, wherein * denotes the point of attachment of L³ to X² in (L-I),
wherein no more than 2 of L¹, X¹, X², L², and L³ can simultaneously be a bond; and
R′ is hydrogen or C₁₋₆ alkyl.

15. The conjugate, or a pharmaceutically acceptable salt, hydrate, solvate, prodrug, stereoisomer, or tautomer thereof, according to claim 14, wherein L³ is selected from the group consisting of a bond, —O—, —C(O)—, —S(O)₂—, C₁₋₆ alkylene, C₂₋₆ alkynylene, and C₁₋₆ heteroalkylene.

16. The conjugate, or a pharmaceutically acceptable salt, hydrate, solvate, prodrug, stereoisomer, or tautomer thereof, according to claim 14 or 15, wherein one of X¹ and X² is not a bond.

17. The conjugate, or a pharmaceutically acceptable salt, hydrate, solvate, prodrug, stereoisomer, or tautomer thereof, according to any one of claims 14-16, wherein one of X¹ and X² is a bond, and the other is a carbocyclyl or heterocyclyl (e.g., piperidinyl and piperazinyl).

18. The conjugate, or a pharmaceutically acceptable salt, hydrate, solvate, prodrug, stereoisomer, or tautomer thereof, according to claim 14 or 15, wherein —X¹-L²-X²— is: 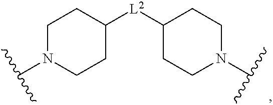
substituted with 0-4 occurrences of R^{b}.

19. The conjugate, or a pharmaceutically acceptable salt, hydrate, solvate, prodrug, stereoisomer, or tautomer thereof, according to claim 14 or 15, wherein —X¹-L²-X²— forms a spiroheterocyclyl having the structure, 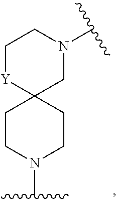
substituted with 0-4 occurrences of R^{b}, wherein Y is selected from CH₂, oxygen, and nitrogen.

20. The conjugate, or a pharmaceutically acceptable salt, hydrate, solvate, prodrug, stereoisomer, or tautomer thereof, according to claim 14 or 15, wherein each of X¹ and X² is independently a bond.

21. The conjugate, or a pharmaceutically acceptable salt, hydrate, solvate, prodrug, stereoisomer, or tautomer thereof, according to any one of claims 4-20, wherein the targeting ligase binder and L2 have a structure of Formula (TLB-L2-I): 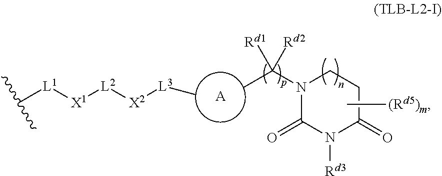
wherein each of L¹, L², L³, X¹, and X² is defined as in claim 14 and each of ring A, R^{d1}, R^{d2}, R^{d3}, R^{d4}, R^{d5}, m, n, and p is defined as in claim 5, and 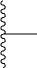
denotes the point of attachment to the targeting ligand in Formula (I-a).

22. The conjugate, or a pharmaceutically acceptable salt, hydrate, solvate, prodrug, stereoisomer, or tautomer thereof, according to any one of claims 4-21, wherein the targeting ligase binder and L2 have a structure selected from the group consisting of Formulas (TLB-L2-I-i), (TLB-L2-I-ii), and (TLB-L2-I-iii): 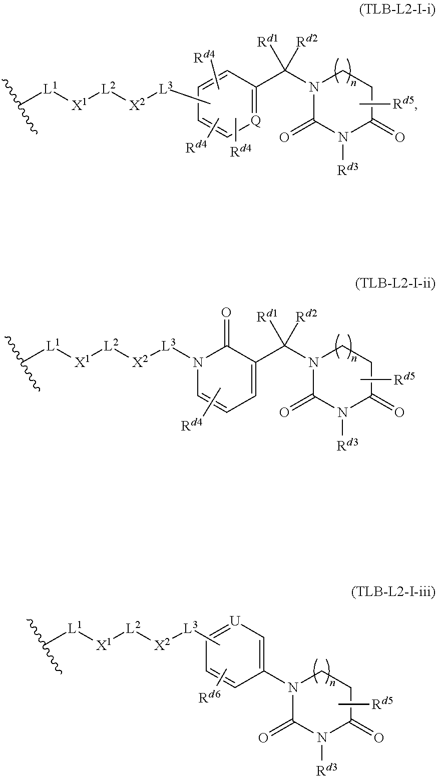
wherein each of L¹, L², L³, X¹, and X² is defined as in claim 14, each of Q, U, R^{d1}, R^{d2}, R^{d3}, R^{d4}, R^{d5}, R^{d6}, m, and n is defined as in claim 8, and 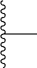
denotes the point of attachment to the targeting ligand in Formula (I-a).

23. The conjugate, or a pharmaceutically acceptable salt, hydrate, solvate, prodrug, stereoisomer, or tautomer thereof, according to any one of claims 4-22, wherein targeting ligase binder and L2 have a structure selected from: 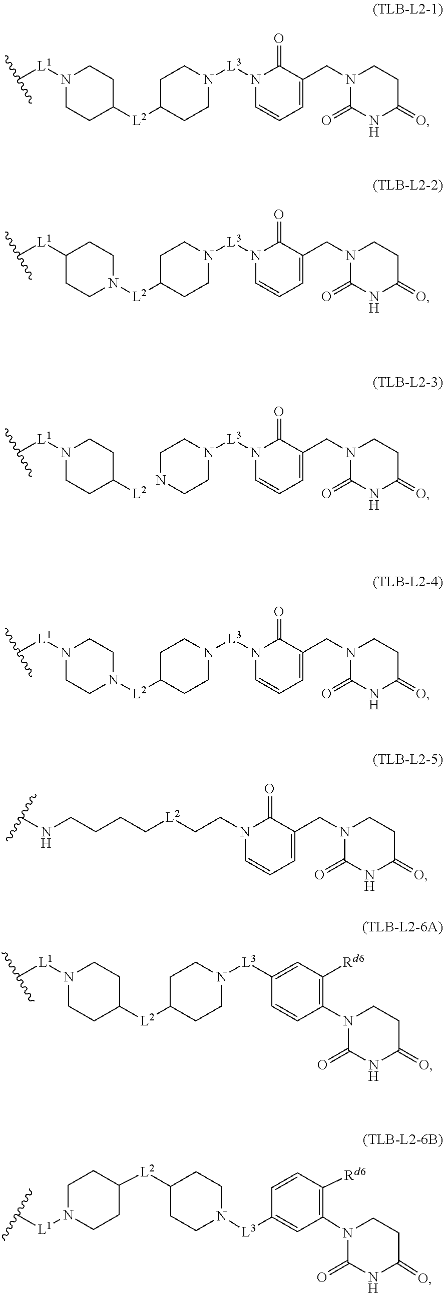 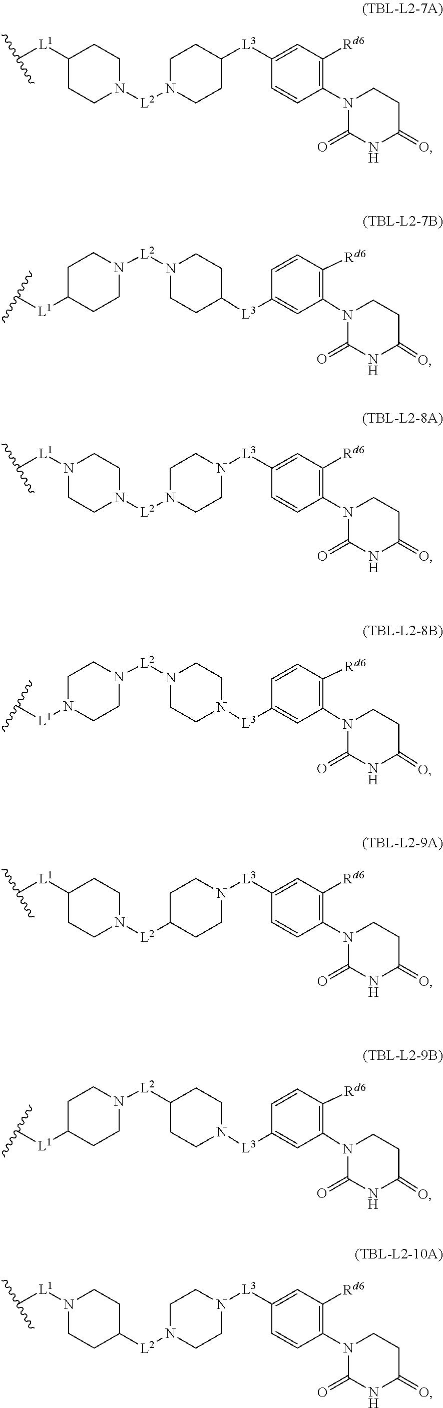 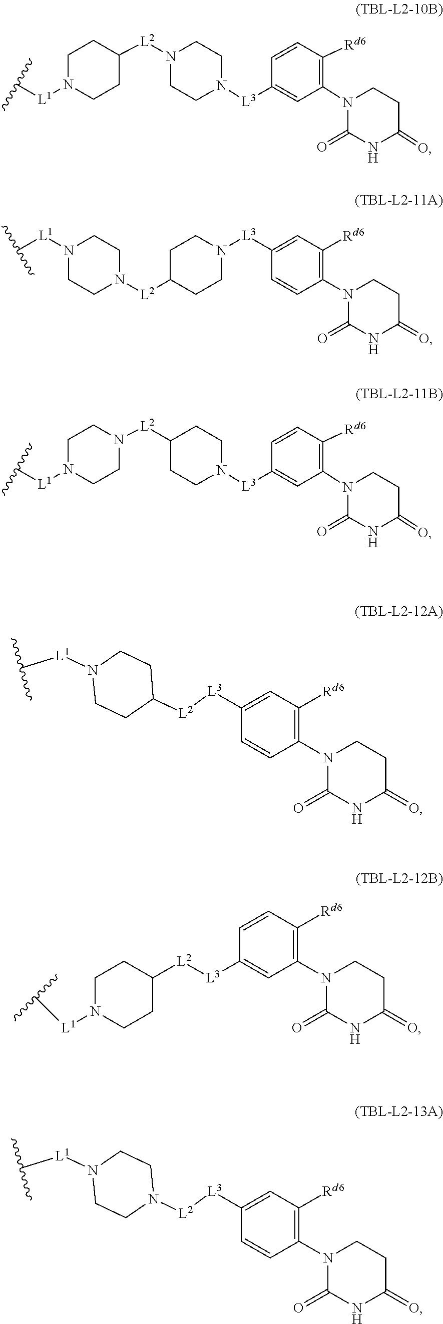 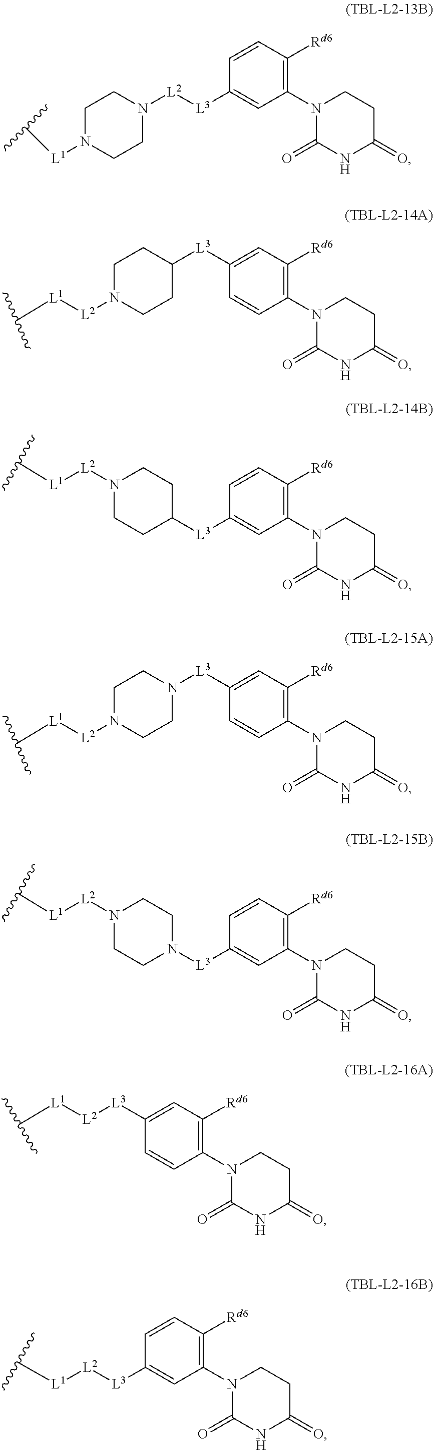 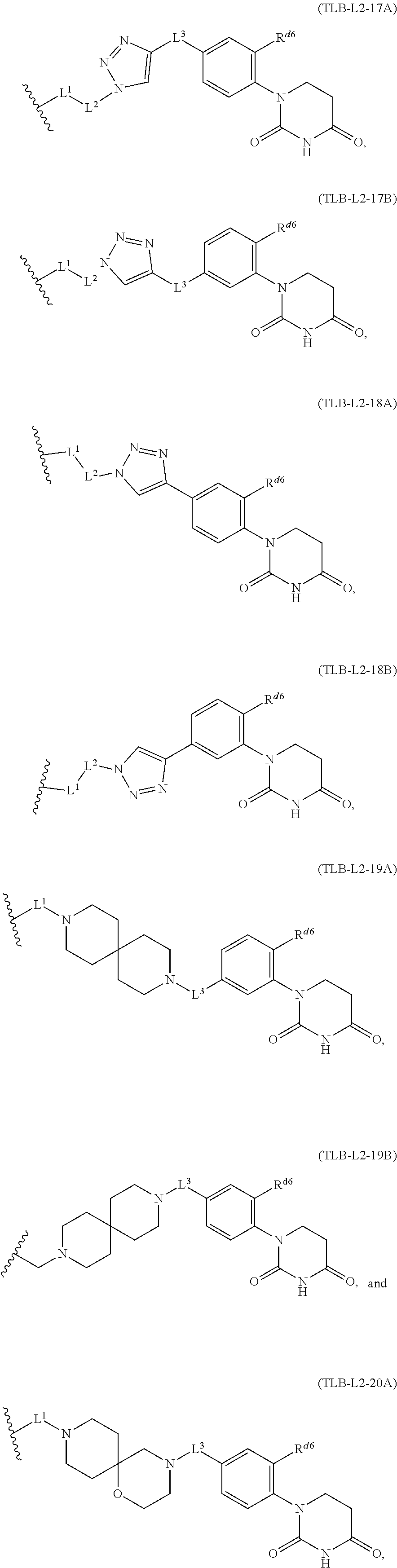
wherein each of L¹, L², L³ is defined as in claim 13 and R^{d6} is as defined in claim 7, and 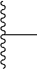
denotes the point of attachment to the targeting ligand in Formula (I-a).

24. The conjugate, or a pharmaceutically acceptable salt, hydrate, solvate, prodrug, stereoisomer, or tautomer thereof, according to any one of claims 1-23, wherein the bifunctional protein degrader (e.g., of Formula (I-a)) has a structure of Formula (BFD-I): 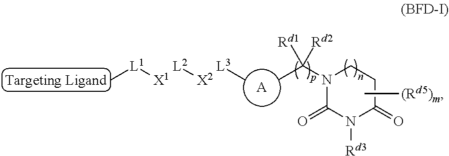
wherein each of L¹, L², L³, X¹, and X² is defined as in claim 13 and each of ring A, R^{d1}, R^{d2}, R^{d3}, R^{d5}, m, n, and p is defined as in claim 4.

25. The conjugate, or a pharmaceutically acceptable salt, hydrate, solvate, prodrug, stereoisomer, or tautomer thereof, according to any one of claims 1-24, wherein the bifunctional protein degrader (e.g., of Formula (I-a)) has a structure selected from the group consisting of Formulas (BFD-I-i), (BFD-I-ii), and (BFD-I-iii): 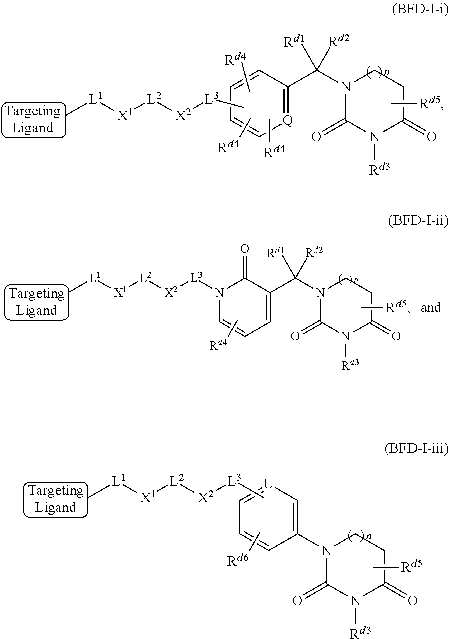
wherein each of L¹, L², L³, X¹, and X² is defined as in claim 13, each of Q, U, R^{d1}, R^{d2}, R^{d3}, R^{d4}, R^{d5}, R^{d6}, m, and n is as defined in claim 7, and the targeting ligand is as defined in claim 3.

26. The conjugate, or a pharmaceutically acceptable salt hydrate, solvate, prodrug, stereoisomer, or tautomer thereof, according to any one of claims 4-25, wherein the targeting ligand binds to a target protein selected from the group listed in Tables 1 or 2.

27. The conjugate, or a pharmaceutically acceptable salt, hydrate, solvate, prodrug, stereoisomer, or tautomer thereof, according to any one of claims 4-26, wherein the targeting ligand is a BTK targeting ligand.

28. The conjugate, or a pharmaceutically acceptable salt, hydrate, solvate, prodrug, stereoisomer, or tautomer thereof, according to any one of claims 4-27, wherein the targeting ligand is a BTK targeting ligand of Formula (BTK-I): 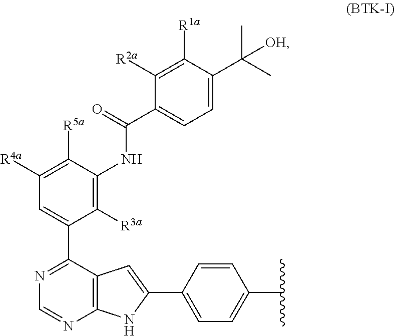
wherein:
R^{1a} is H or halo;
R^{2a} is halo;
R^{3a} is C₁₋₆ alkyl;
R^{4a} is halo;
R^{5a} is H or halo; and 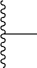
denotes the point of attachment to L2 in Formula (I-a).

29. The conjugate, or a pharmaceutically acceptable salt, hydrate, solvate, prodrug, stereoisomer, or tautomer thereof, according to any one of claims 1-27, wherein the bifunctional protein degrader (e.g., of Formula (I-a)) has a structure of Formula (BFD-BTK-I): 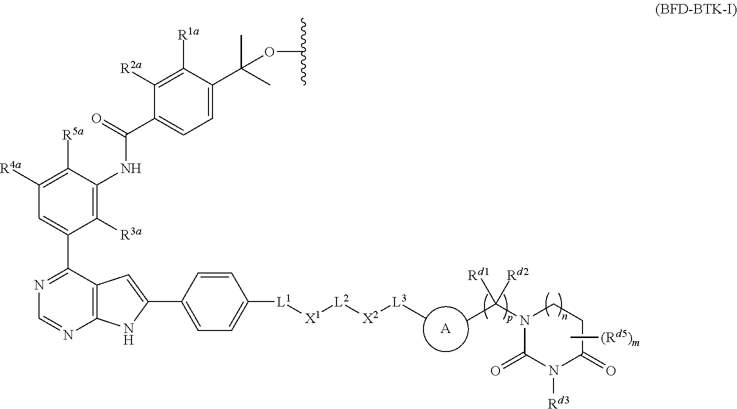
wherein each of L¹, L², L³, X¹, and X² is defined as in claim 14, each of ring A, R^{d1}, R^{d2}, R^{d3}, R^{d5}, m, n, and p is as defined in claim 5, each of R^{1a}, R^{2a}, R^{3a}, R^{4a}, and R^{5a} is defined as in claim 28, and 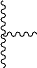
denotes the point of attachment to L1 in Formula (I).

30. The conjugate, or a pharmaceutically acceptable salt, hydrate, solvate, prodrug, stereoisomer, or tautomer thereof, according to any one of claims 1 and 3-29, wherein L1 is covalently linked to the bifunctional degrader compound.

31. The conjugate, or a pharmaceutically acceptable salt, hydrate, solvate, prodrug, stereoisomer, or tautomer thereof, according to any one of claims 1 and 3-30, wherein L1 is covalently linked to the solubilizing domain when present.

32. The conjugate, or a pharmaceutically acceptable salt, hydrate, solvate, prodrug, stereoisomer, or tautomer thereof, according to any one of claims 1 and 3-31, wherein L1 is degraded or hydrolyzed at physiological conditions.

33. The conjugate, or a pharmaceutically acceptable salt, hydrate, solvate, prodrug, stereoisomer, or tautomer thereof, according to any one of claims 1 and 3-32, wherein L1 is pH sensitive (e.g., acid labile or base labile).

34. The conjugate, or a pharmaceutically acceptable salt, hydrate, solvate, prodrug, stereoisomer, or tautomer thereof, according to any one of claims 1 and 3-33, wherein L1 is cleaved through the action of an enzyme.

35. The conjugate, or a pharmaceutically acceptable salt, hydrate, solvate, prodrug, stereoisomer, or tautomer thereof, according to claim 34, wherein the rate of hydrolysis of L1 is increased by at least 0.5 fold (e.g., at least 1, 1.5, 2, 2.5, 3, 4, 5, 7.5, 10, 12.5, 15, 20, 25, 50, 75, 100, 250, 500, 750, 1000 or more) compared with the rate of hydrolysis of L1 in the absence of an enzyme.

36. The conjugate, or a pharmaceutically acceptable salt, hydrate, solvate, prodrug, stereoisomer, or tautomer thereof, according to claim 34 or 35, wherein L1 comprises a bond cleavable in a cell (e.g., a cell organelle) or serum, e.g., of a sample or subject.

37. The conjugate, or a pharmaceutically acceptable salt, hydrate, solvate, prodrug, stereoisomer, or tautomer thereof, according to claim 34 or 35, wherein the enzyme is an esterase.

38. The conjugate, or a pharmaceutically acceptable salt, hydrate, solvate, prodrug, stereoisomer, or tautomer thereof, according to any one of claims 1 and 3-37, wherein L1 comprises an ester, phosphate, disulfide, thiol, hydrazone, ether, or amide.

39. The conjugate, or a pharmaceutically acceptable salt, hydrate, solvate, prodrug, stereoisomer, or tautomer thereof, according to any one of claims 1 and 3-38, wherein L1 comprises an ester.

40. The conjugate, or a pharmaceutically acceptable salt, hydrate, solvate, prodrug, stereoisomer, or tautomer thereof, according to any one of claims 1 and 3-39, wherein L1 has the structure of Formula (L1-I): 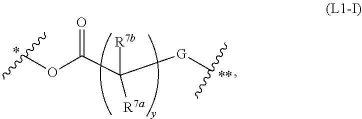
wherein each of R^{7a} and R^{7b} is independently selected from the group consisting of H, C₁₋₆ alkyl, C₁₋₆ haloalkyl, C₁₋₆ heteroalkyl, cycloalkyl, and halo; G is C₁₋₆ alkyl, C₁₋₆ heteroalkyl, —NR′— wherein R′ is H, C₁₋₆ alkyl, or —(CH₂)₁₋₂—C(O)₂H, 1 to 5 natural or unnatural amino acids, cycloalkyl, heterocyclyl, aryl, or heteroaryl, wherein each alkyl, heteroalkyl, cycloalkyl, heterocyclyl, aryl, or heterocyclyl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; and each “*” and “**” independently denote the point of attachment to the bifunctional protein degrader or solubilizing domain, when present, or the fatty acid in Formula (I) or (I′).

41. The conjugate, or a pharmaceutically acceptable salt, hydrate, solvate, prodrug, stereoisomer, or tautomer thereof, according to any one of claims 1 and 3-40, wherein the structure of L1 is selected from the group consisting of:

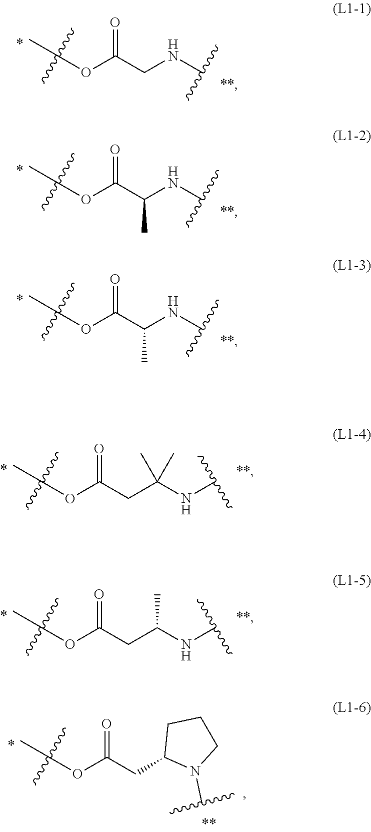

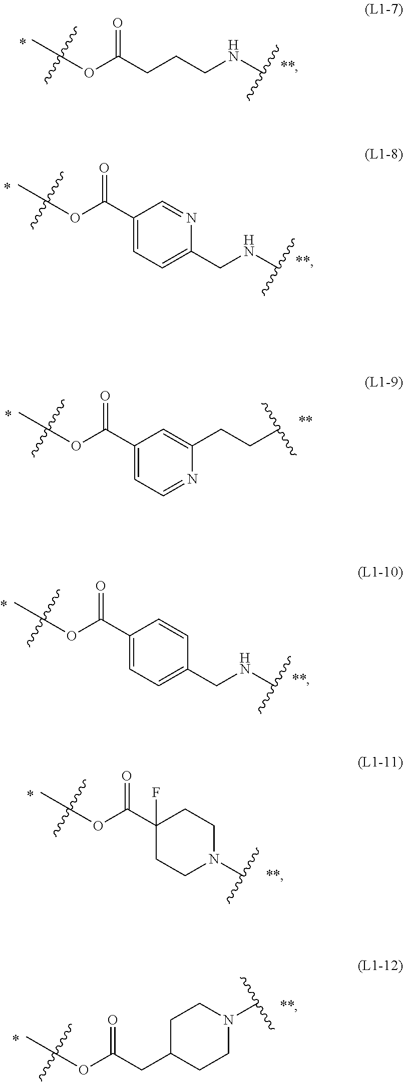 [SMILES: *NCCCC(=O)O* ; *NCc1ccc(C(=O)O*)cn1 ; *CCc1cc(C(=O)O*)ccn1 ; *NCc1ccc(C(=O)O*)cc1 ; *OC(=O)C1(F)CCN(*)CC1 ; *OC(=O)CC1CCN(*)CC1]

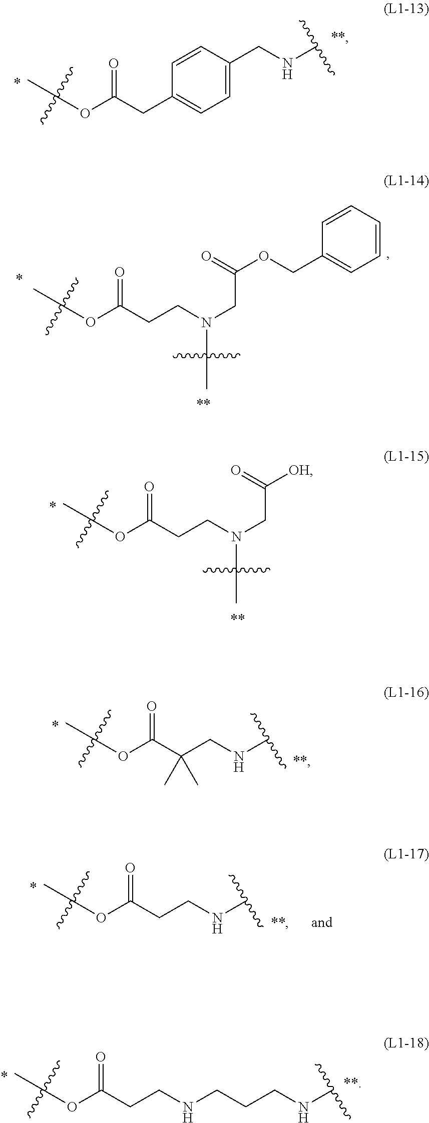

42. The conjugate, or a pharmaceutically acceptable salt, hydrate, solvate, prodrug, stereoisomer, or tautomer thereof, according to any one of claims 1 and 3-41, wherein the bifunctional degrader compound and L1 have the structure of Formula (BFD-L1-I): 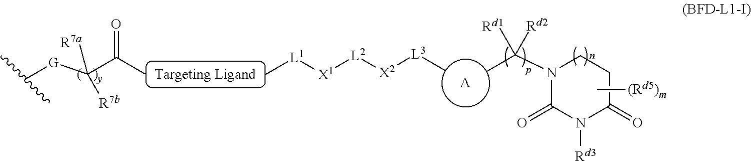
wherein each of L¹, L², L³, X¹, and X² is defined as in claim 14; each of ring A, R^{d1}, R^{d2}, R^{d3}, R^{d5}, m, n, and p is as defined in claim 5; each of R^{7a}, R^{7b}, G, and y is as defined in claim 41; and 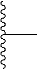
denotes the point of attachment to the solubilizing domain, when present, in Formula (I) or (I′).

43. The conjugate, or a pharmaceutically acceptable salt, hydrate, solvate, prodrug, stereoisomer, or tautomer thereof, according to any one of claims 1 and 3-38, wherein the bifunctional degrader compound and L1 have the structure of Formula (BFD-L1-II): 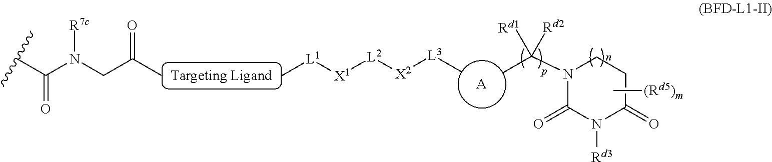
wherein each of L¹, L², L³, X¹, and X² is defined as in claim 14; each of ring A, R^{d1}, R^{d2}, R^{d3}, R^{d5}, m, n, and p is as defined in claim 5; R^{7c} is H, C₁₋₆ alkyl, C₁₋₆ haloalkyl, C₁₋₆ heteroalkyl, C₃₋₆ cycloalkyl; and 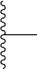
denotes the point of attachment to the solubilizing domain, when present, in Formula (I) or (I′).

44. The conjugate, or a pharmaceutically acceptable salt, hydrate, solvate, prodrug, stereoisomer, or tautomer thereof, according to any one of claims 1-43, wherein the solubilizing domain, when present, comprises a water-soluble monomer or polymer, e.g., to increase one or more of amphiphilicity, hydrophilicity, water-solubility, pH sensitivity, or stability.

45. The conjugate, or a pharmaceutically acceptable salt, hydrate, solvate, prodrug, stereoisomer, or tautomer thereof, according to claim 44, wherein the solubilizing domain, when present, comprises a water-soluble polymer.

46. The conjugate, or a pharmaceutically acceptable salt, hydrate, solvate, prodrug, stereoisomer, or tautomer thereof, according to any one of claims 1-45, wherein the solubilizing domain, when present, comprises a polyalkylene or polyheteroalkylene moiety.

47. The conjugate, or a pharmaceutically acceptable salt, hydrate, solvate, prodrug, stereoisomer, or tautomer thereof, according to any one of claims 1-46, wherein the solubilizing domain, when present, comprises a polyethylene glycol (PEG), a polyethylene oxide (PEO), a polypropylene glycol (PPG), a polyglycerol (PG), a poloxamine (POX), a polybutylene oxide (PBO), polylactic acid (PLA), polyglycolic acid (PGA), poly(lactic-co-glycolic acid) (PLGA), polycaprolactone (PCL), polydioxanone (PDO), a polyanhydride, a polyacrylide, a polyvinyl, or a polyorthoester.

48. The conjugate, or a pharmaceutically acceptable salt, hydrate, solvate, prodrug, stereoisomer, or tautomer thereof, according to any one of claims 1-47, wherein the solubilizing domain, when present, comprises a polyethylene glycol (PEG).

49. The conjugate, or a pharmaceutically acceptable salt, hydrate, solvate, prodrug, stereoisomer, or tautomer thereof, according to any one of claims 1-48, wherein the water-soluble polymer is between 100 Da to about 20,000 Da in size.

50. The conjugate, or a pharmaceutically acceptable salt, hydrate, solvate, prodrug, stereoisomer, or tautomer thereof, according to any one of claims 1-49, wherein the water-soluble polymer is between 200 Da to about 1,000 Da in size.

51. The conjugate, or a pharmaceutically acceptable salt, hydrate, solvate, prodrug, stereoisomer, or tautomer thereof, according to any one of claims 1-50, wherein the solubilizing domain, when present, has a structure selected from the group consisting of Formulas (SD-I), (SD-II), and (SD-III): 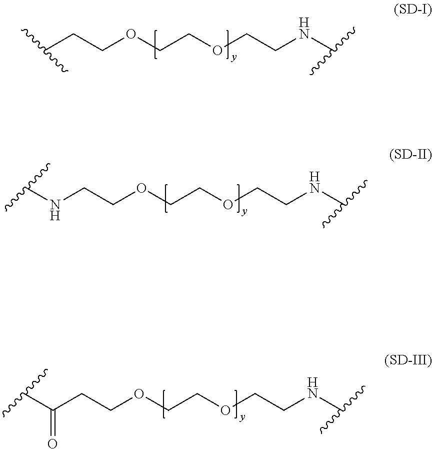
wherein y is an integer between 0 to 35; and 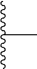
denotes the points of attachment to L1 and the fatty acid in Formula (I).

52. The conjugate, or a pharmaceutically acceptable salt, hydrate, solvate, prodrug, stereoisomer, or tautomer thereof, according to claim 51, wherein y is 5 to 30, e.g., 6 to 20, e.g., 7 to 15, e.g., 9 to 13, or e.g., 11.

53. The conjugate, or a pharmaceutically acceptable salt, hydrate, solvate, prodrug, stereoisomer, or tautomer thereof, according to any one of claims 1-52, wherein the solubilizing domain, when present, has the structure of Formula (SD-I): 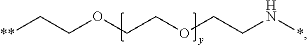
wherein * indicates the point of attachment to the fatty acid, ** indicates the point of attachment to L1, and y is 11.

54. The conjugate, or a pharmaceutically acceptable salt, hydrate, solvate, prodrug, stereoisomer, or tautomer thereof, according to any one of claims 1 and 3-53, wherein the bifunctional degrader compound, L1, and solubilizing domain, when present, have the structure of Formula (BFD-L1-SD-I): 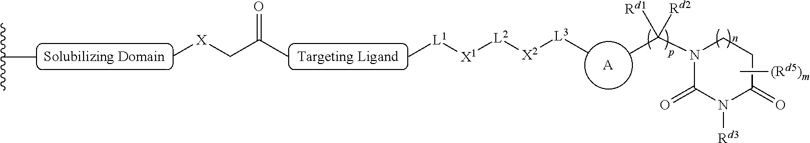
wherein each of L¹, L², L³, X¹, and X² is defined as in claim 14; each of ring A, R^{d1}, R^{d2}, R^{d3}, R^{d5}, m, n, and p is as defined in claim 5; R^{7c} is H, C₁₋₆ alkyl, C₁₋₆ haloalkyl, C₁₋₆ heteroalkyl, C₃₋₆ cycloalkyl; X is O, S, C(R^{7a})(R^{7b}), or N(R^{7c})C(O); and 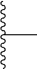
denotes the point of attachment to the fatty acid in Formula (I).

55. The conjugate, or a pharmaceutically acceptable salt, hydrate, solvate, prodrug, stereoisomer, or tautomer thereof, according to any one of claims 1 and 3-54, wherein the bifunctional degrader compound, L1, and solubilizing domain, when present, have the structure of Formula (BFD-L1-SD-Ia): 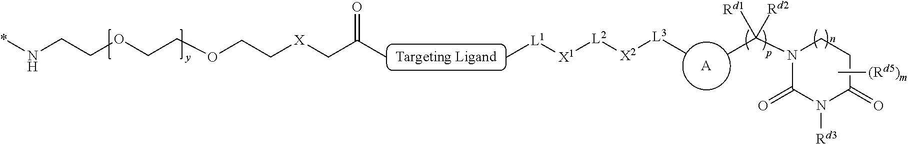
wherein each of L¹, L², L³, X¹, and X² is defined as in claim 13; each of ring A, R^{d1}, R^{d2}, R^{d3}, R^{d5}, m, n, and p is as defined in claim 4; R^{7c} is H, C₁₋₆ alkyl, C₁₋₆ haloalkyl, C₁₋₆ heteroalkyl, C₃₋₆ cycloalkyl; X is O, S, C(R^{7a})(R^{7b}), or N(R^{7c})C(O); y is an integer between 0 and 35; and * indicates the point of attachment to the fatty acid.

56. The conjugate, or a pharmaceutically acceptable salt, hydrate, solvate, prodrug, stereoisomer, or tautomer thereof, according to any one of claims 1-55, wherein the fatty acid has a structure selected from the group consisting of Formula (FA-1), Formula (FA-2), and Formula (FA-3): 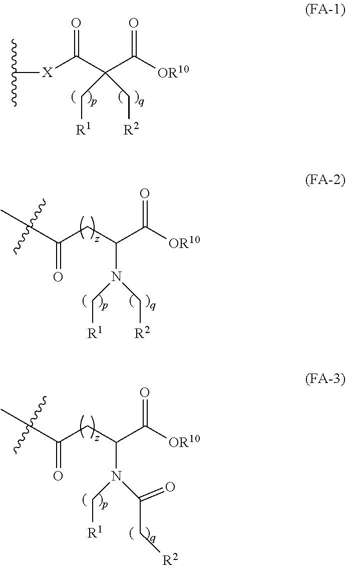
wherein:
X is O or N(R³);
p and q are each an integer independently selected from 5 to 30;
z is an integer selected from 0 to 5;
R¹ and R² are each independently selected from CH₃, OR¹⁰, C(O)OR¹⁰, and P(O)(OR¹⁰)₂;
R³ and R¹⁰ are each independently H or C₁₋₆ alkyl; and 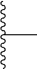
denotes the point of attachment to the solubilizing domain, when present, or fatty acid in Formula (I).

57. The conjugate, or a pharmaceutically acceptable salt, hydrate, solvate, prodrug, stereoisomer, or tautomer thereof, according to claim 56, wherein the fatty acid has a structure of Formula (FA-1).

58. The conjugate, or a pharmaceutically acceptable salt, hydrate, solvate, prodrug, stereoisomer, or tautomer thereof, according to claim 56 or 57, wherein the fatty acid of Formula (FA-1) has a structure selected from Formula (FA-1a) and (FA-1b): 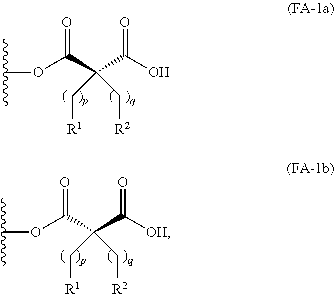
wherein:
p and q are each an integer independently selected from 5 to 30;
R¹ and R² are each independently selected from CH₃, OR¹⁰, C(O)OR¹⁰, and P(O)(OR¹⁰)₂;
R¹⁰ are each independently H or C₁₋₆ alkyl; and
* denotes the point of attachment to the solubilizing domain, when present, in Formula (I).

59. The conjugate, or a pharmaceutically acceptable salt, hydrate, solvate, prodrug, stereoisomer, or tautomer thereof, according to claim 56, wherein the fatty acid has a structure of Formula (FA-2).

60. The conjugate, or a pharmaceutically acceptable salt, hydrate, solvate, prodrug, stereoisomer, or tautomer thereof, according to any one of claim 56 or 59, wherein the fatty acid of Formula (FA-2) has a structure selected from Formula (FA-2a) and (FA-2b): 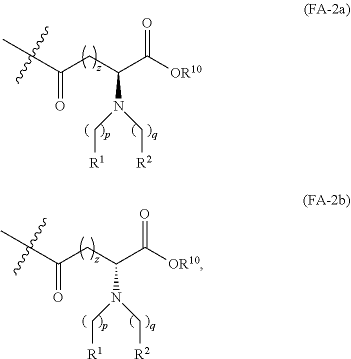
wherein:
p and q are each an integer independently selected from 5 to 30;
z is an integer selected from 0 to 5;
R¹ and R² are each independently selected from CH₃, OR¹⁰, C(O)OR¹⁰, and P(O)(OR¹⁰)₂;
R¹⁰ is H or C₁₋₆ alkyl; and 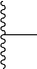
denotes the point of attachment to the solubilizing domain, when present, in Formula (I).

61. The conjugate, or a pharmaceutically acceptable salt, hydrate, solvate, prodrug, stereoisomer, or tautomer thereof, according to any one of claims 1-60 selected from: 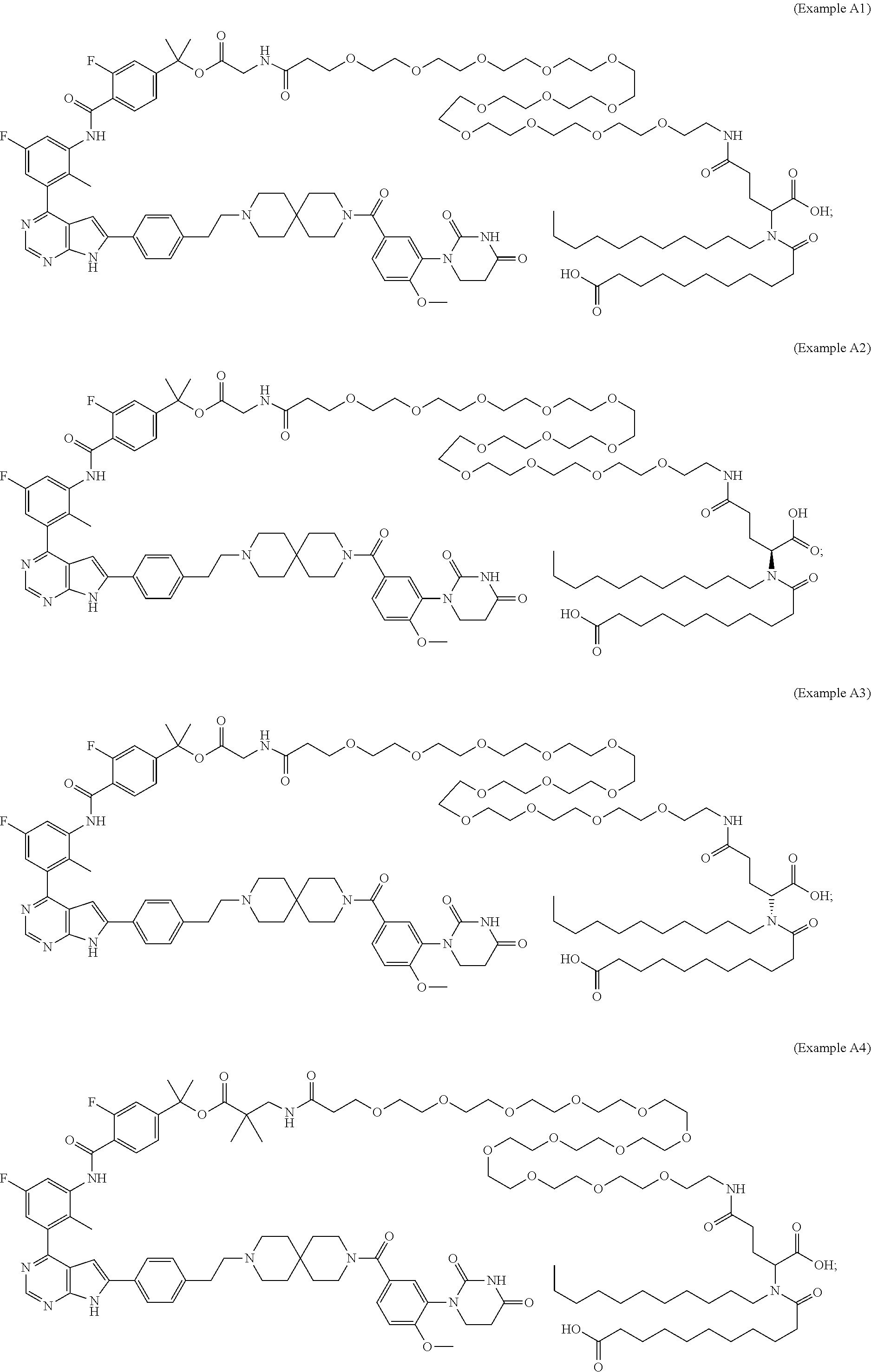 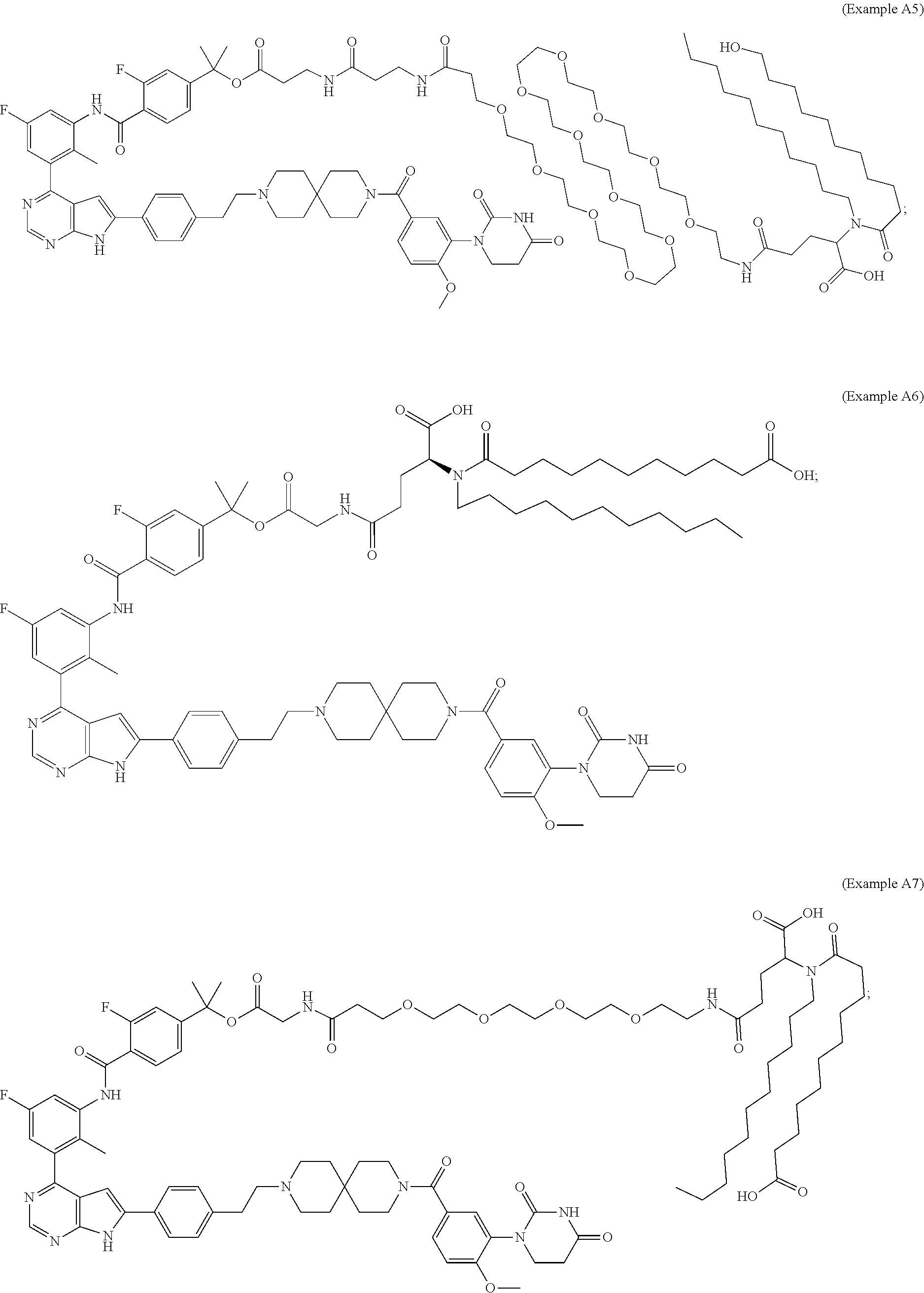 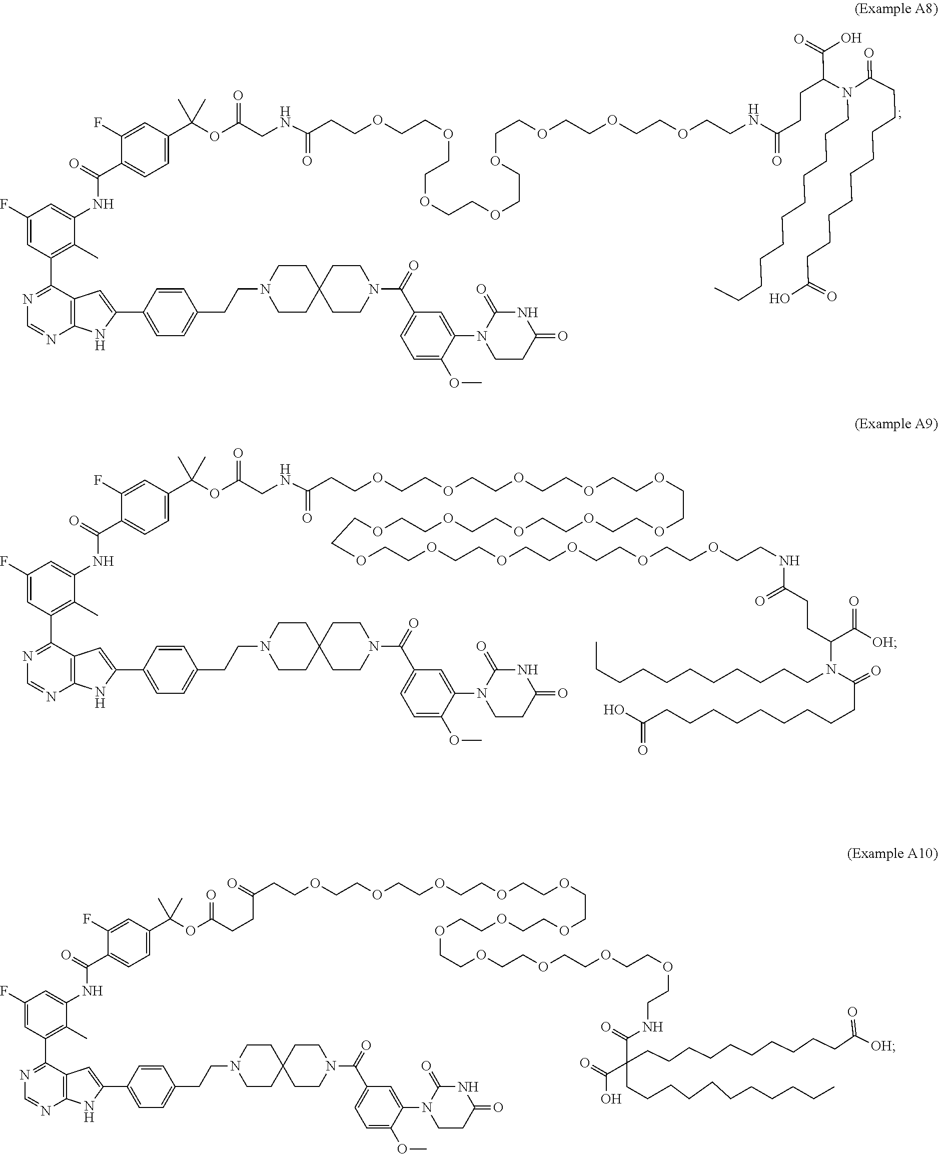 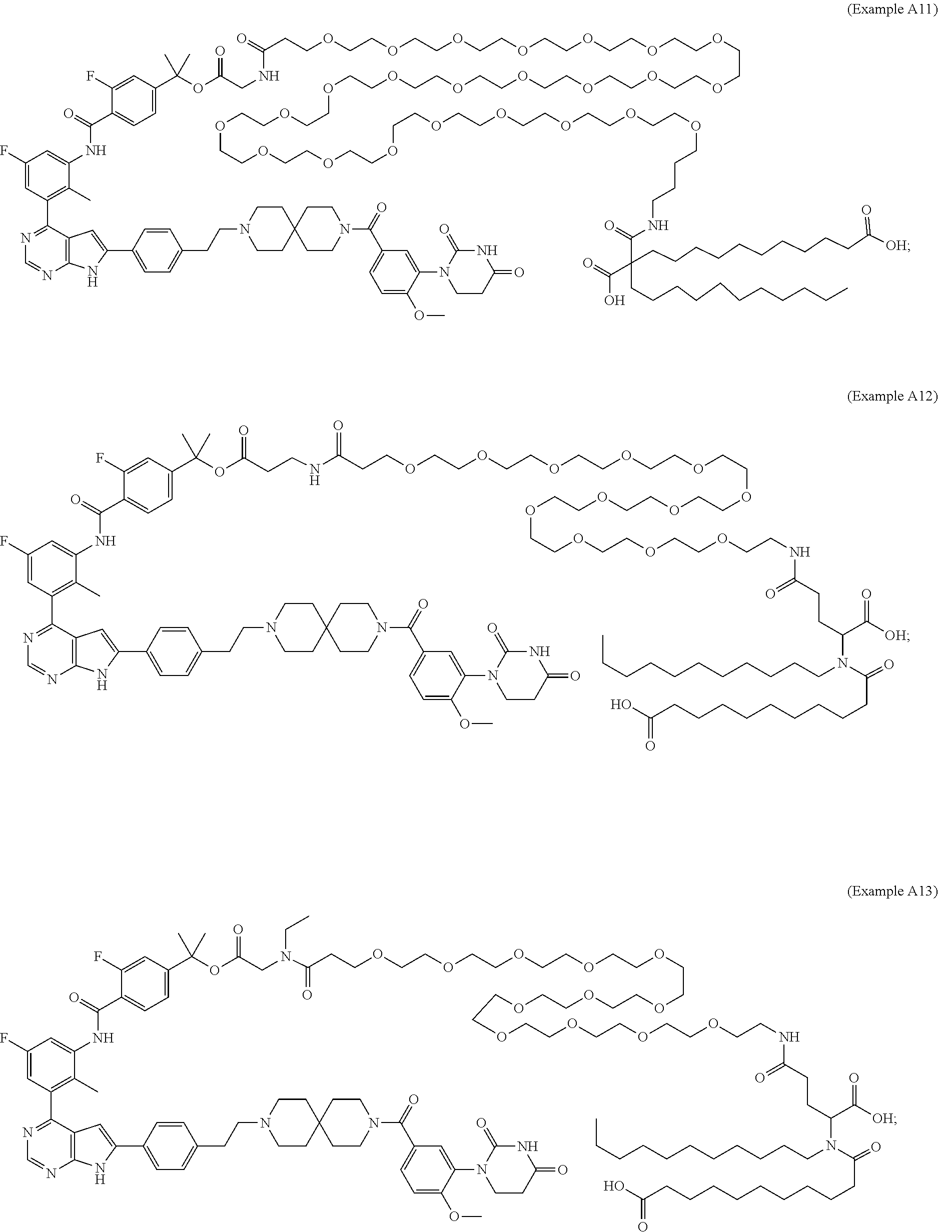 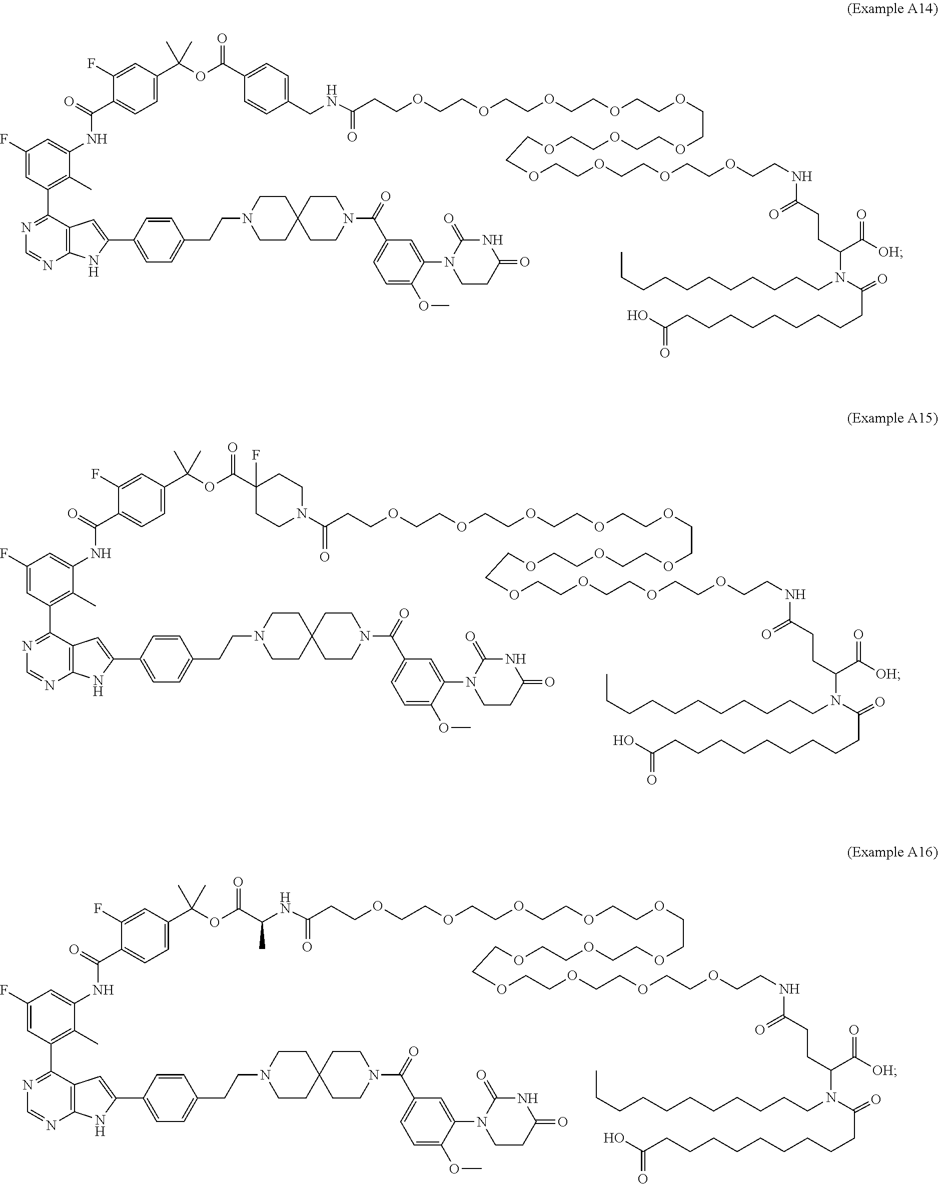 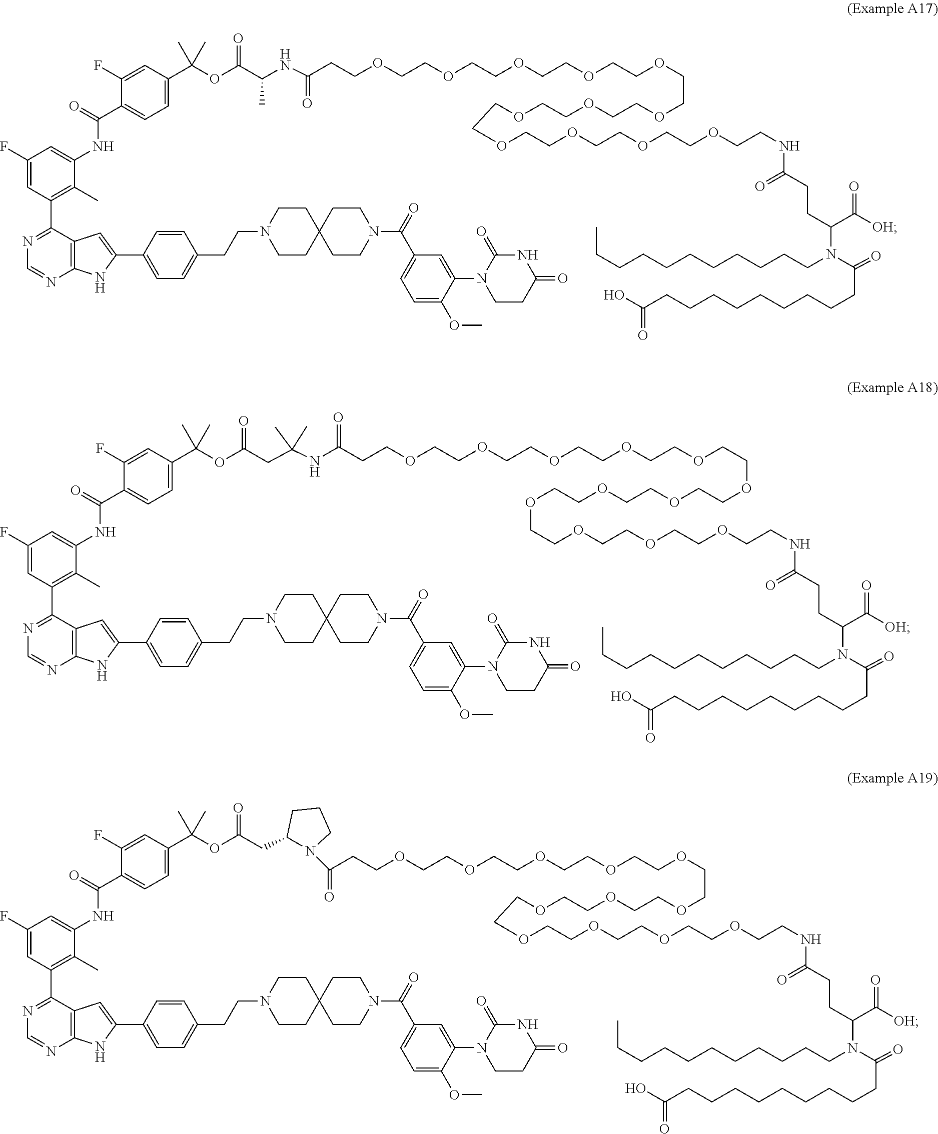 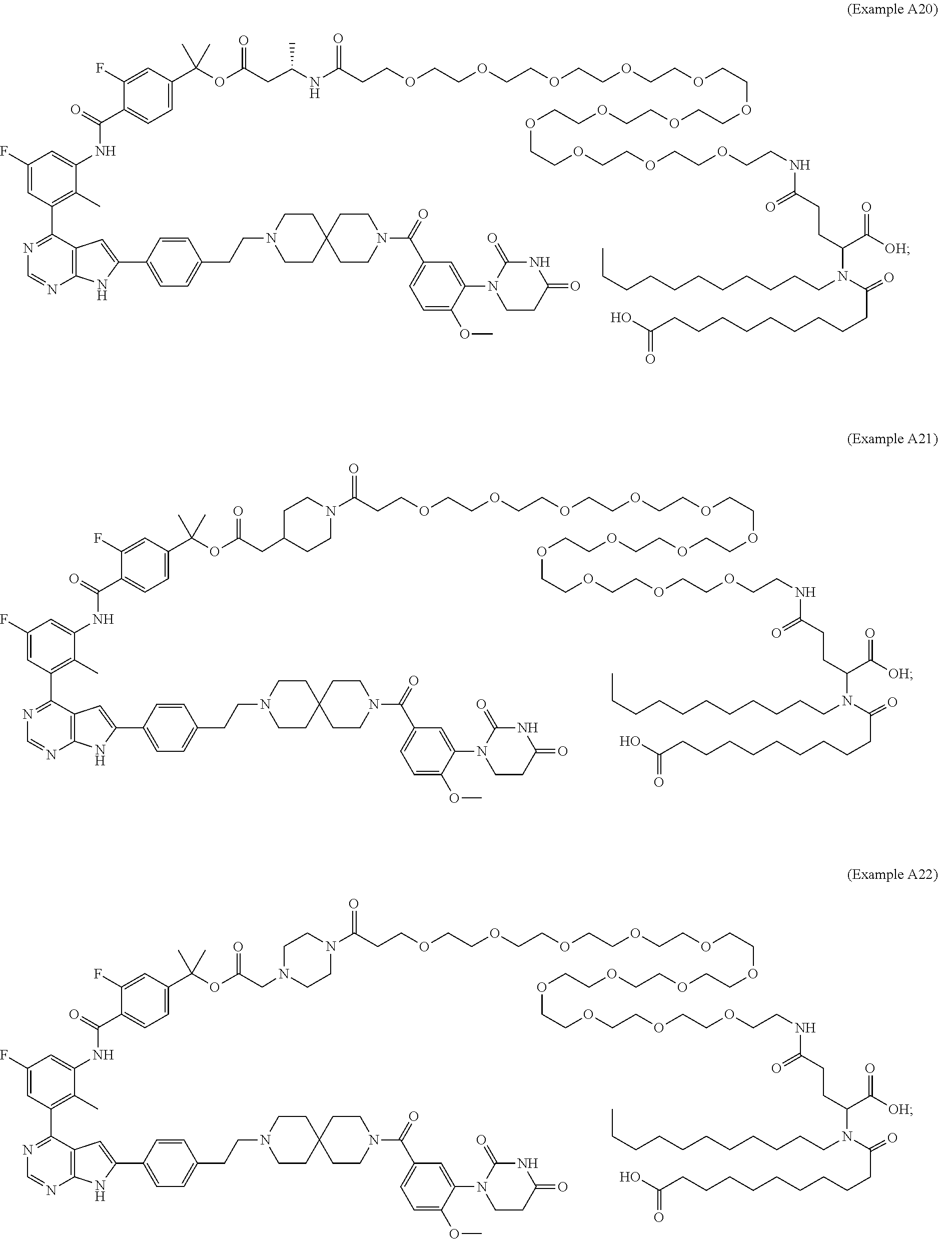 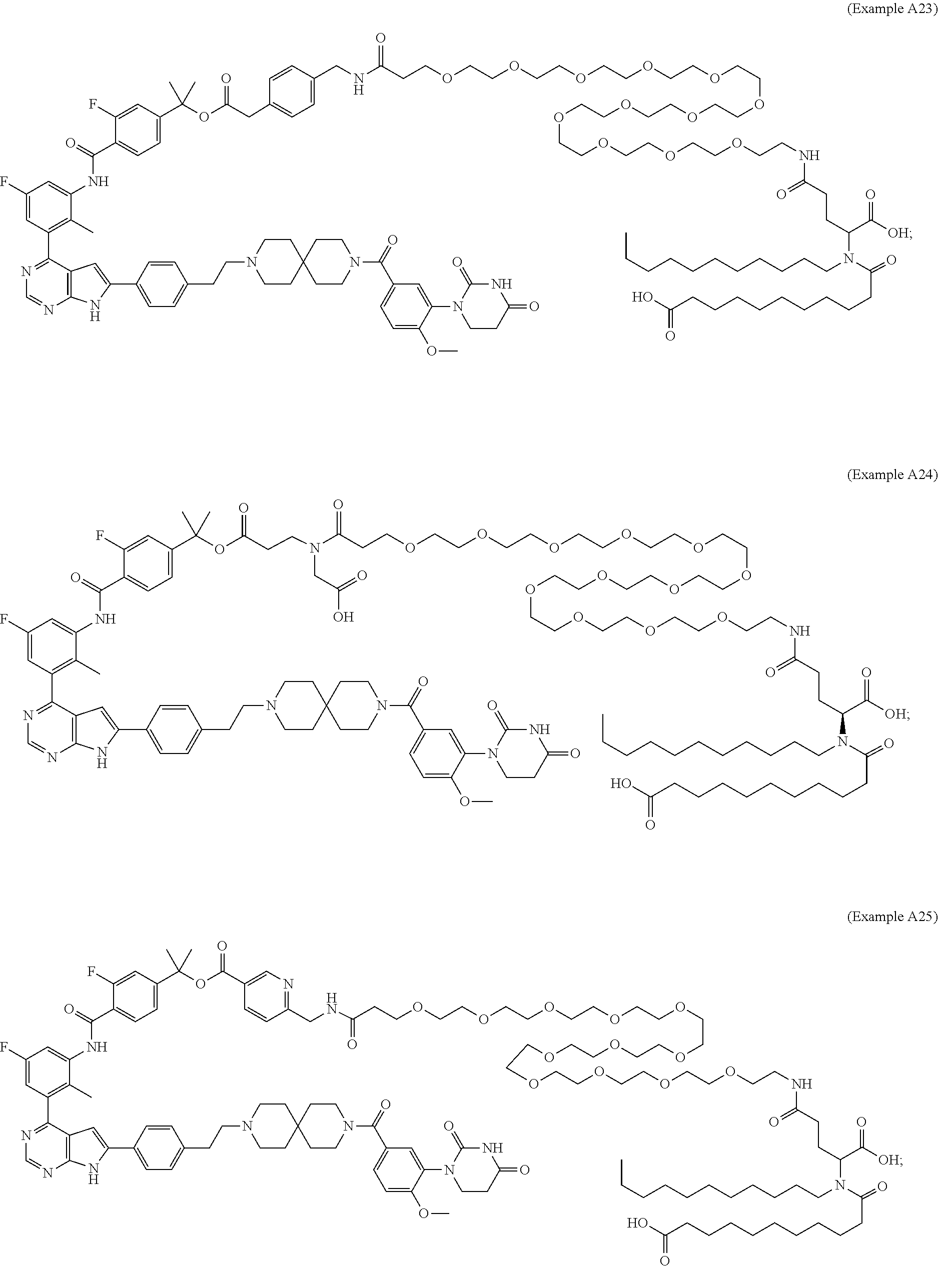 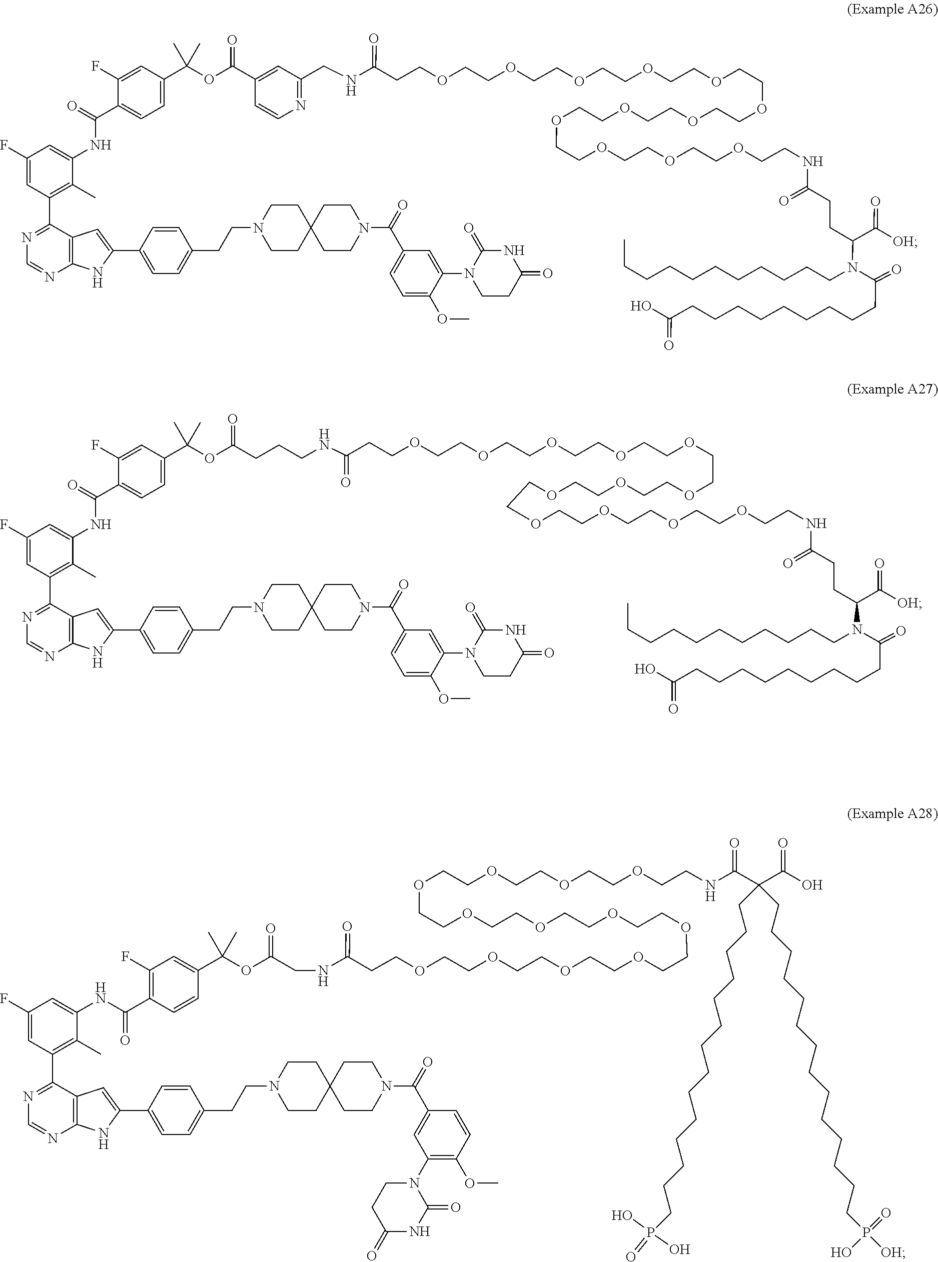 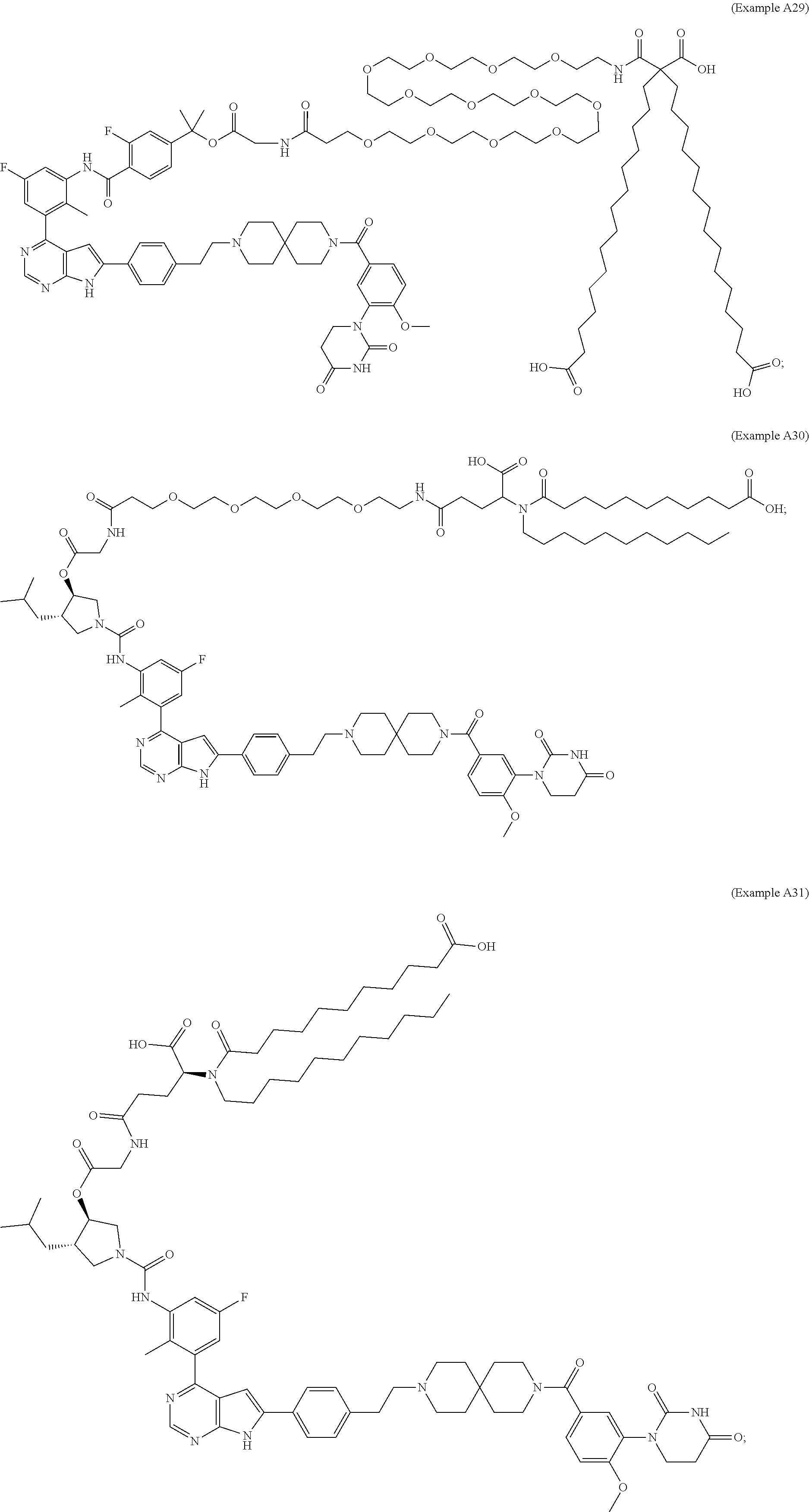 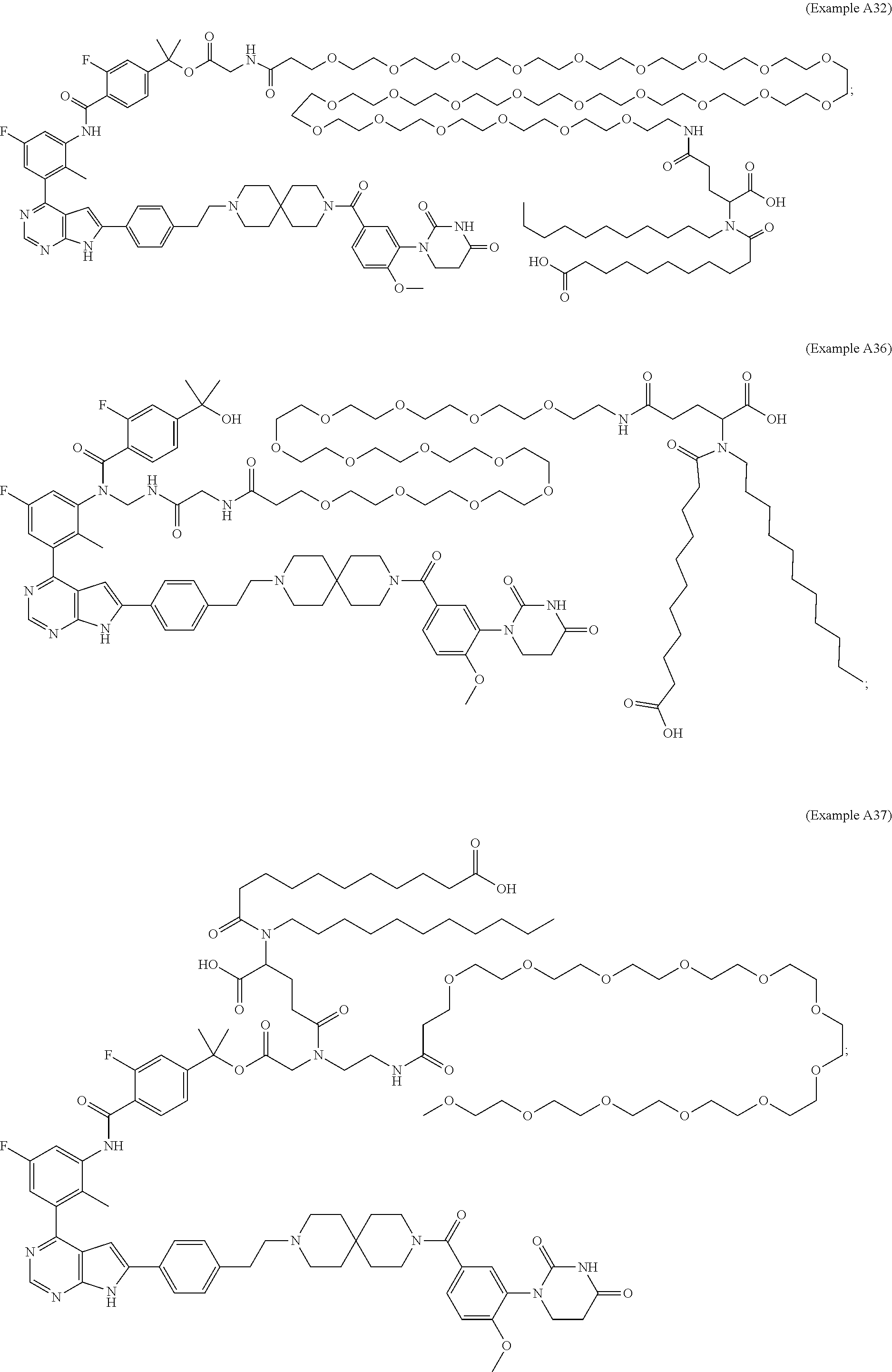 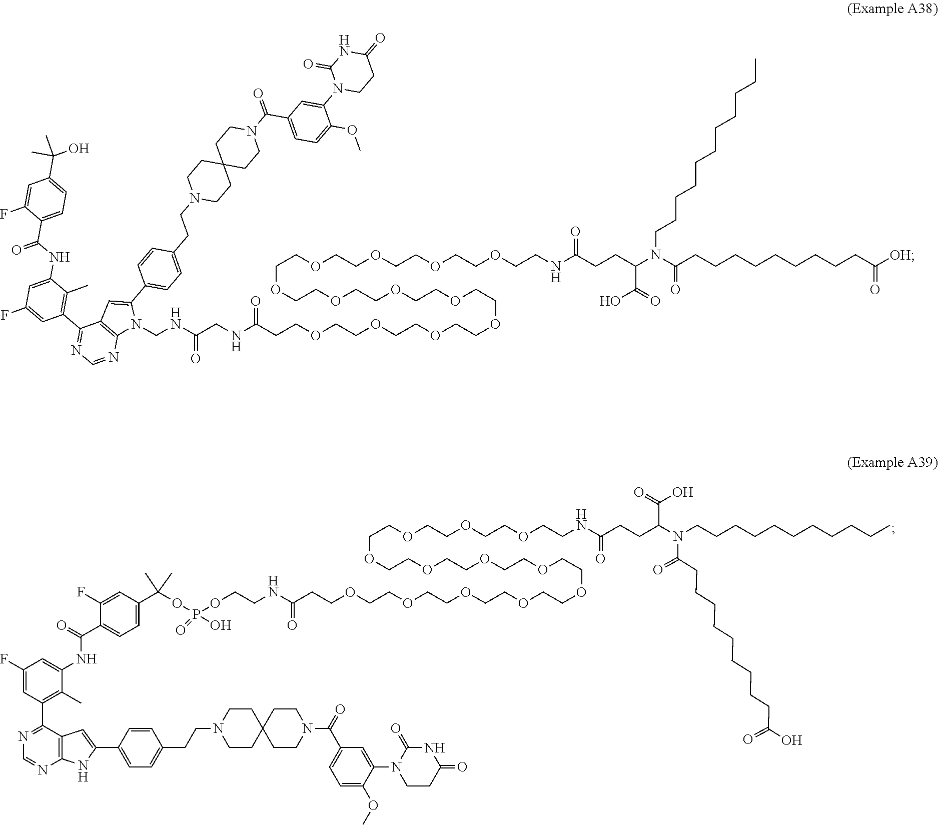 [SMILES: CCCCCCCCCCCN(C(=O)CCCCCCCCCC(=O)O)C(CCC(=O)NCCOCCOCCOCCOCCOCCOCCOCCOCCOCCOCCOCCOCCC(=O)NCC(=O)NCn1c(-c2ccc(CCN3CCC4(CC3)CCN(C(=O)c3ccc(OC)c(N5CCC(=O)NC5=O)c3)CC4)cc2)cc2c(-c3cc(F)cc(NC(=O)c4ccc(C(C)(C)O)cc4F)c3C)ncnc21)C(=O)O ; CCCCCCCCCCCN(C(=O)CCCCCCCCCC(=O)O)C(CCC(=O)NCCOCCOCCOCCOCCOCCOCCOCCOCCOCCOCCOCCOCCC(=O)NCCOP(=O)(O)OC(C)(C)c1ccc(C(=O)Nc2cc(F)cc(-c3ncnc4[nH]c(-c5ccc(CCN6CCC7(CC6)CCN(C(=O)c6ccc(OC)c(N8CCC(=O)NC8=O)c6)CC7)cc5)cc34)c2C)c(F)c1)C(=O)O] 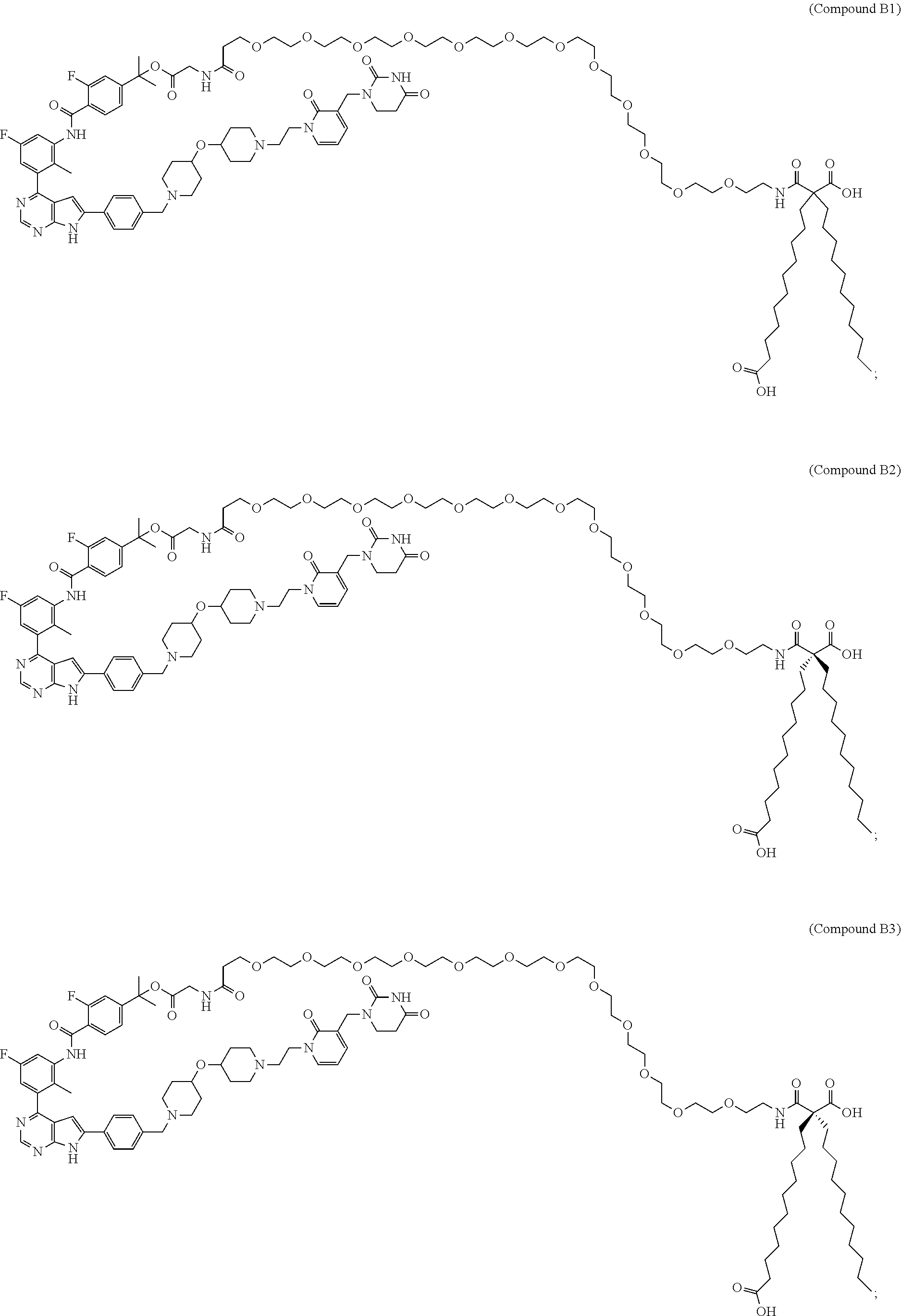 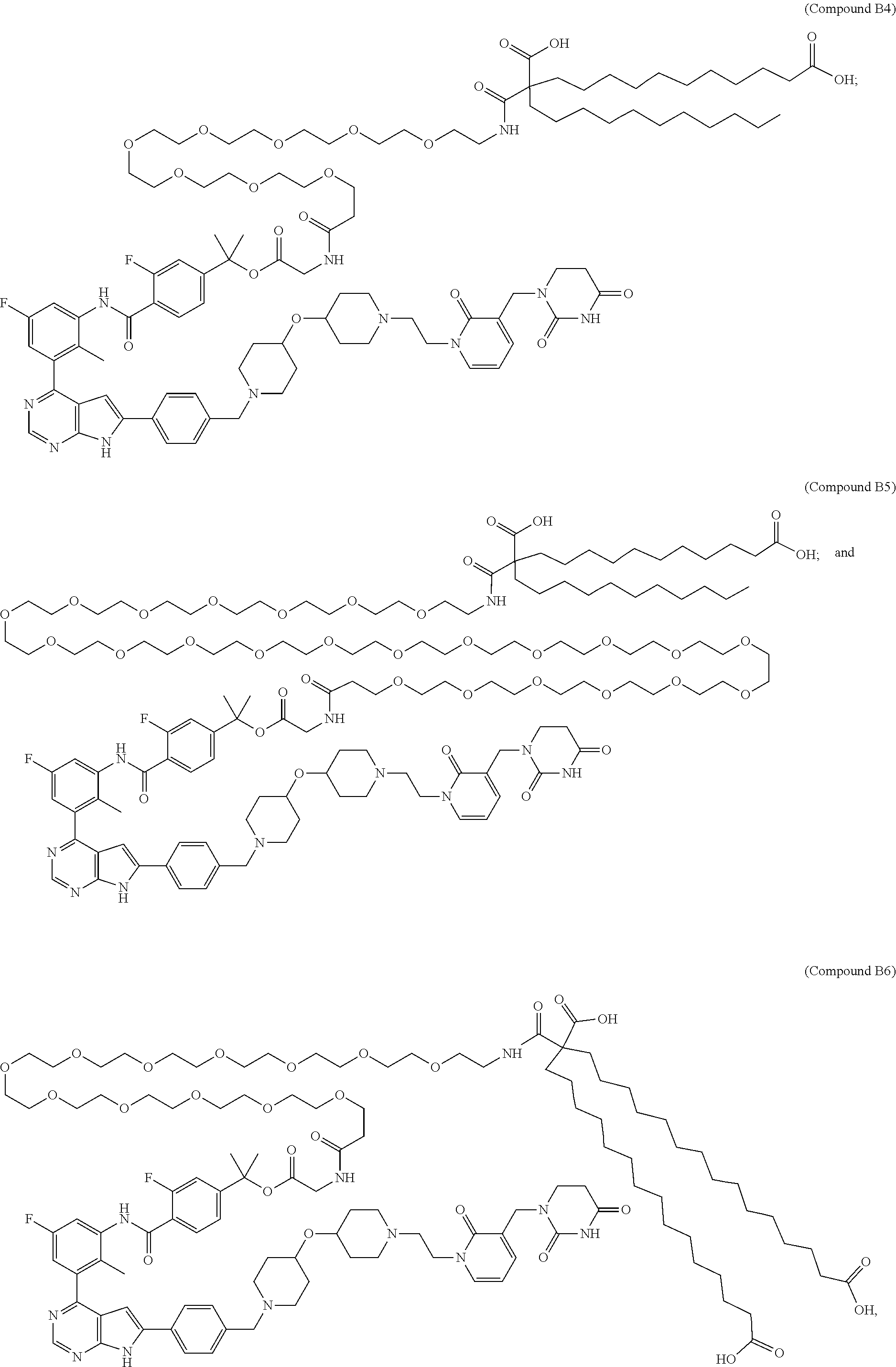
or a pharmaceutically acceptable salt, hydrate, solvate, prodrug, stereoisomer, or tautomer thereof.

62. The conjugate, or a pharmaceutically acceptable salt, hydrate, solvate, prodrug, stereoisomer, or tautomer thereof, according to any of the preceding claims, wherein the conjugate of Formula (I) or (I) has a plasma stability half-life of more than 10 hours, e.g., more than 20 hours, e.g., more than 30 hours.

63. The conjugate, or a pharmaceutically acceptable salt, hydrate, solvate, prodrug, stereoisomer, or tautomer thereof, according to any one of claims 1 and 3-62, wherein the improvement of plasma stability compared to the non-conjugated bifunctional degrader compound without L1, the solubilizing domain, when present, and the fatty acid is at least 2 fold, e.g., at least 5 fold, at least 10 fold, at least 20 fold, at least 30 fold, at least 40 fold, at least 50 fold, or at least 75 fold.

64. A pharmaceutical composition comprising a conjugate, or a pharmaceutically acceptable salt, hydrate, solvate, prodrug, stereoisomer, or tautomer thereof according to any one of the preceding claims and one or more pharmaceutically acceptable carriers.

65. The pharmaceutical composition of claim 64, which allows for maintenance of the conjugate, or a pharmaceutically acceptable salt, hydrate, solvate, prodrug, stereoisomer, or tautomer thereof of any one of claims 1-63 in the serum for at least 1, 2, 3, 4, 5, 6, 7, 8, 9, 10, 11, 12, 13, 14, 15, 16, 17, 18, 19, 20, or 21 days.

66. The pharmaceutical composition of claim 64 or 65, formulated for subcutaneous or intravenous administration.

67. A combination comprising a conjugate, or a pharmaceutically acceptable salt, hydrate, solvate, prodrug, stereoisomer, or tautomer, according to any one of claims 1-63 and one or more therapeutically active agents.

68. A method of degrading a protein target in a subject, wherein the method comprises administering to the subject a therapeutically effective amount of the conjugate, or a pharmaceutically acceptable salt, hydrate, solvate, prodrug, stereoisomer, or tautomer thereof, according to any one of claims 1-63.

69. A method of treating or preventing a disease in a subject in need thereof, wherein the method comprises administering to the subject a therapeutically effective amount of the conjugate, or a pharmaceutically acceptable salt, hydrate, solvate, prodrug, stereoisomer, or tautomer thereof, of any one of claims 1-63.

70. The method of claim 69, wherein the disease is cancer.

71. The method of claim 70, wherein the cancer is selected from chronic lymphocytic leukemia (CLL), mantle cell lymphoma (MCL), small lymphocytic lymphoma (SLL), Waldenstrom's macroglobulinemia, diffuse large B-cell lymphoma (DLBCL), follicular lymphoma, Burkitt lymphoma, Marginal Zone Lymphoma, immunoblastic large cell lymphoma, Richter Syndrome, and precursor B-lymphoblastic lymphoma, primary and secondary multiple myeloma, B-cell prolymphocytic leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, lymphomatoid granulomatosis, and acute lymphoblastic leukemia, Hodgkin lymphoma, non-Hodgkin lymphoma, post-transplant lymphoproliferative disorder, hairy cell leukemia, and Histiocytic and dendritic neoplasms.

72. A conjugate, or a pharmaceutically acceptable salt, hydrate, solvate, prodrug, stereoisomer, or tautomer thereof, according to any one of claims 1-63 for use as a medicament.

73. A conjugate, or a pharmaceutically acceptable salt, hydrate, solvate, prodrug, stereoisomer, or tautomer thereof, according to any one of claims 1-63 for use in the treatment of a disease.

74. The use according to claim 73, wherein the disease is cancer.

75. The use according to claim 74, wherein the cancer is selected from chronic lymphocytic leukemia (CLL), mantle cell lymphoma (MCL), small lymphocytic lymphoma (SLL), Waldenstrom's macroglobulinemia, diffuse large B-cell lymphoma (DLBCL), follicular lymphoma, Burkitt lymphoma, Marginal Zone Lymphoma, immunoblastic large cell lymphoma, Richter Syndrome, and precursor B-lymphoblastic lymphoma, primary and secondary multiple myeloma, B-cell prolymphocytic leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, lymphomatoid granulomatosis, and acute lymphoblastic leukemia, Hodgkin lymphoma, non-Hodgkin lymphoma, post-transplant lymphoproliferative disorder, hairy cell leukemia, and Histiocytic and dendritic neoplasms.

76. Use of a conjugate, or a pharmaceutically acceptable salt, hydrate, solvate, prodrug, stereoisomer, or tautomer thereof, according to any one of claims 1-63 in the manufacture of a medicament for the treatment of a disease mediated by BTK.

77. The use according to claim 76, wherein the disease is cancer.

78. The use according to claim 77, wherein the cancer is selected from chronic lymphocytic leukemia (CLL), mantle cell lymphoma (MCL), small lymphocytic lymphoma (SLL), Waldenstrom's macroglobulinemia, diffuse large B-cell lymphoma (DLBCL), follicular lymphoma, Burkitt lymphoma, Marginal Zone Lymphoma, immunoblastic large cell lymphoma, Richter Syndrome, and precursor B-lymphoblastic lymphoma, primary and secondary multiple myeloma, B-cell prolymphocytic leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, lymphomatoid granulomatosis, and acute lymphoblastic leukemia, Hodgkin lymphoma, non-Hodgkin lymphoma, post-transplant lymphoproliferative disorder, hairy cell leukemia, and Histiocytic and dendritic neoplasms.
